(19) United States
(12) Patent Application Publication
(10) Pub. No.: US 2026/0159831 A1
(43) Pub. Date: Jun. 11, 2026

(54) ENGINEERED CONSTRUCTS FOR INCREASED TRANSCRIPTION OF RNA PAYLOADS

(71) Applicant: SHAPE THERAPEUTICS INC., Seattle, WA (US)

(72) Inventors: Stephen BURLEIGH, Seattle, WA (US); Duankun LEE, Seattle, WA (US); Adrian Wrangham BRIGGS, Seattle, WA (US)

(21) Appl. No.: 19/105,265

(22) PCT Filed: Aug. 23, 2023
(86) PCT No.: PCT/US2023/030982

§ 371 Date: Feb. 20, 2025

Related U.S. Application Data
(60) Provisional application No. 63/400,583, filed on Aug. 24, 2022.
(60) Provisional application No. 63/419,889, filed on Oct. 27, 2022.
(60) Provisional application No. 63/453,584, filed on Mar. 21, 2023.
(60) Provisional application No. 63/466,625, filed on May 15, 2023.

(51) Int. Cl. C12N 15/11; A61K 48/00; C12N 9/78; C12N 15/113; C12N 15/86

(52) U.S. Cl. CPC C12N 15/11; A61K 48/005; C12N 9/78; C12N 15/113; C12N 15/86; C12N 2310/11; C12N 2310/20; C12N 2750/14143

(57) ABSTRACT

Described herein are expression cassettes encoding small RNA payloads, such as engineered guide RNAs. The expression cassettes may be engineered to increase expression of the small RNA payload encoded by the expression cassette. The engineered expression cassettes include various sequence elements that may enhance expression of the small RNA payload, such as transcription factor binding sequences, transcriptional termination sequences, and core promoter sequences. Sequence elements may be combined or interchanged to tune small RNA payload expression levels. Also described herein are methods of editing a target gene using a small RNA payload encoded by an expression cassette.

### CROSS-REFERENCE

This application claims the benefit of U.S. Provisional Application No. 63/400,583, entitled “ENGINEERED CONSTRUCTS FOR INCREASED TRANSCRIPTION OF RNA PAYLOADS,” filed Aug. 24, 2022, U.S. Provisional Application No. 63/419,889, entitled “ENGINEERED CONSTRUCTS FOR INCREASED TRANSCRIPTION OF RNA PAYLOADS,” filed Oct. 27, 2022, U.S. Provisional Application No. 63/453,584, entitled “ENGINEERED CONSTRUCTS FOR INCREASED TRANSCRIPTION OF RNA PAYLOADS,” filed Mar. 21, 2023, and U.S. Provisional Application No. 63/466,625, entitled “ENGINEERED CONSTRUCTS FOR INCREASED TRANSCRIPTION OF RNA PAYLOADS,” filed May 15, 2023, which applications are incorporated herein by reference in their entireties.

### SEQUENCE LISTING

The instant application contains a Sequence Listing which has been submitted electronically in eXtensible Markup Language (XML) format and is hereby incorporated by reference in its entirety. Said XML copy, created on Aug. 21, 2023, is named “421688-712021_SL.xml” and is 1.29 megabytes in size.

### BACKGROUND

A wide variety of diseases and disorders are caused by mutations, deletions, altered expression, or altered splicing of genes. RNAs can serve as a mechanism for gene therapy, such as by editing a mutated RNA sequence associated with a disease. There is a need for expression cassettes to increase or modulate expression of RNA payloads.

### SUMMARY

In various aspects, the present disclosure provides an expression cassette comprising: a promoter sequence comprising a sequence having at least 80% sequence identity to any one of: a) SEQ ID NO: 17, SEQ ID NO: 1250, or SEQ ID NO: 1262; b) SEQ ID NO: 13 or SEQ ID NO: 15; or c) SEQ ID NO: 1241, SEQ ID NO: 1251, SEQ ID NO: 1252, SEQ ID NO: 1253, or SEQ ID NO: 1263; a payload sequence under transcriptional control of the promoter sequence, the payload sequence comprising a small RNA payload; and a termination sequence comprising a sequence having at least 80% identity to any one of: a) SEQ ID NO: 1002, SEQ ID NO: 1017, SEQ ID NO: 1264, or SEQ ID NO: 1265; or b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269.

In various aspects, the present disclosure provides an expression cassette comprising: a promoter sequence comprising a sequence having at least 80% sequence identity to any one of: a) SEQ ID NO: 17, SEQ ID NO: 1250, or SEQ ID NO: 1262; b) SEQ ID NO: 13 or SEQ ID NO: 15; or c) SEQ ID NO: 1241, SEQ ID NO: 1251, SEQ ID NO: 1252, SEQ ID NO: 1253, or SEQ ID NO: 1263; a payload sequence under transcriptional control of the promoter sequence, the payload sequence comprising a small RNA payload; and a termination sequence.

In various aspects, the present disclosure provides an expression cassette comprising: a promoter sequence; a payload sequence under transcriptional control of the promoter sequence, the payload sequence comprising a small RNA payload; and a termination sequence comprising a sequence having at least 80% identity to any one of: a) SEQ ID NO: 1002, SEQ ID NO: 1017, SEQ ID NO: 1264, or SEQ ID NO: 1265; or b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269.

In various aspects, the present disclosure provides an expression cassette comprising: a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263; a payload sequence under transcriptional control of the promoter sequence, the payload sequence comprising a small RNA payload; and a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289.

In various aspects, the present disclosure provides an expression cassette comprising: a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263; a payload sequence under transcriptional control of the promoter sequence, the payload sequence comprising a small RNA payload; and a termination sequence.

In various aspects, the present disclosure provides an expression cassette comprising: a promoter sequence; a payload sequence under transcriptional control of the promoter sequence, the payload sequence comprising a small RNA payload; and a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289.

In some aspects, the promoter sequence comprises a sequence having at least 90% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263. In some aspects, the promoter sequence comprises a sequence having at least 95% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263.

In some aspects, the termination sequence comprises a sequence having at least 90% sequence identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289. In some aspects, the termination sequence comprises a sequence having at least 95% sequence identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289.

In some aspects, the promoter sequence comprises SEQ ID NO: 17. In some aspects, the promoter sequence comprises SEQ ID NO: 1262. In some aspects, the promoter sequence comprises SEQ ID NO: 1250. In some aspects, the promoter sequence comprises SEQ ID NO: 1251. In some aspects, the promoter sequence comprises SEQ ID NO: 1252. In some aspects, the promoter sequence comprises SEQ ID NO: 1253.

In some aspects, the termination sequence comprises SEQ ID NO: 1264. In some aspects, the termination sequence comprises SEQ ID NO: 1265. In some aspects, the termination sequence comprises SEQ ID NO: 1254. In some aspects, the termination sequence comprises SEQ ID NO: 1255. In some aspects, the termination sequence comprises SEQ ID NO: 1257. In some aspects, the termination sequence comprises SEQ ID NO: 60. In some aspects, the termination sequence comprises SEQ ID NO: 1242. In some aspects, the termination sequence comprises SEQ ID NO: 1269. In some aspects, the termination sequence comprises SEQ ID NO: 1017.

In some aspects, the small RNA payload comprises an engineered guide RNA capable of hybridizing to a target sequence. In some aspects, the engineered guide RNA is at least 70%, at least 75%, at least 80%, at least 85%, at least 90%, at least 95%, or 100% reverse complementary to the target sequence. In some aspects, the engineered guide RNA comprises at least one base pair mismatch relative to the target sequence. In some aspects, the target sequence comprises an adenosine residue. In some aspects, the target sequence is an RNA sequence. In some aspects, the RNA sequence is a mRNA or a pre-mRNA.

In some aspects, the target sequence comprises a G to A mutation relative to a wild type sequence. In some aspects, the target sequence comprises a missense mutation or a nonsense mutation relative to a wild type sequence. In some aspects, the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2).

In some aspects, the payload sequence comprises at least 80%, at least 85%, at least 90%, at least 95%, or 100% sequence identity to SEQ ID NO: 1273, SEQ ID NO: 1274, or SEQ ID NO: 61. In some aspects, the small RNA payload comprises an antisense oligonucleotide, an siRNA, an shRNA, a miRNA, or a tracrRNA. In some aspects, the small RNA payload is not less than 20 nucleotide residues and not more than 500 nucleotide residues long. In some aspects, the small RNA payload is not less than 60 and not more than 100 residues long. In some aspects, the small RNA payload is not less than 80 and not more than 120 residues long. In some aspects, the small RNA payload is not less than 100 and not more than 140 residues long. In some aspects, the small RNA payload is not less than 130 and not more than 170 residues long. In some aspects, the payload sequence further comprises an Sm binding sequence or a hairpin sequence. In some aspects, the hairpin sequence comprises a U7 hairpin. In some aspects, the hairpin sequence comprises at least 80%, at least 85%, at least 90%, at least 95%, or 100% sequence identity to SEQ ID NO: 52 or SEQ ID NO: 54, or the Sm binding sequence comprises at least 80%, at least 85%, at least 90%, at least 95%, or 100% sequence identity to SEQ ID NO: 56 or SEQ ID NO: 58.

In some aspects, the expression cassette has a length of not less than 1300 nucleotide residues and not more than 2160 nucleotide residues. In some aspects, the expression cassette comprises at least 80% sequence identity to a U1 sequence or a U7 sequence. In some aspects, the U1 sequence is a mouse U1 sequence or a human U1 sequence. In some aspects, the U7 sequence is a mouse U7 sequence or a human U7 sequence.

In some aspects, the promoter sequence comprises a zinc finger 143 motif capable of recruiting a ZNF143 transcription factor. In some aspects, the promoter sequence comprises an OCT-1 transcription factor binding sequence capable of recruiting an OCT-1 transcription factor. In some aspects, the promoter sequence comprises a proximal sequence element capable of recruiting a SNAPc. In some aspects, the proximal sequence element is capable of integrator dependent recruitment of RNA polymerase II.

In some aspects, the small RNA payload is capable of forming a guide-target RNA scaffold comprising a structural feature upon hybridization of the small RNA payload to a target sequence. In some aspects, the structural feature is a bulge, a mismatch, an internal loop, a hairpin, or combinations thereof. In some aspects, the structural feature comprises the bulge, and wherein the bulge is a symmetric bulge. In some aspects, the structural feature comprises the bulge, and wherein the bulge is an asymmetric bulge. In some aspects, the structural feature comprises the internal loop, and wherein the internal loop is a symmetric internal loop. In some aspects, the structural feature comprises the internal loop, and wherein the internal loop is an asymmetric internal loop. In some aspects, the structural feature comprises the hairpin, and wherein the hairpin is a recruitment hairpin or a non-recruitment hairpin. In some aspects, the guide-target RNA scaffold comprises a Wobble base pair.

In various aspects, the present disclosure provides a recombinant polynucleotide encoding one or more of the expression cassettes as described herein.

In some aspects, the recombinant polynucleotide encodes two of the expression cassettes as described herein comprising a first promoter, a second promoter, a first termination sequence, and a second termination sequence. In some aspects, the first promoter and the second promoter are the same. In some aspects, the first promoter and the second promoter are different. In some aspects, the first termination sequence and the second termination sequence are the same. In some aspects, the first termination sequence and the second termination sequence are different. In some aspects, the first promoter comprises SEQ ID NO: 17. In some aspects, the second promoter comprises SEQ ID NO: 1262. In some aspects, the first termination sequence comprises SEQ ID NO: 1264. In some aspects, the second termination sequence comprises SEQ ID NO: 1265. In some aspects, (a) the first promotor sequence comprises SEQ ID NO: 17, the first termination sequence comprises SEQ ID NO: 1264, the second promotor sequence comprises SEQ ID NO: 1262 and the second termination sequence comprises SEQ ID NO: 1265; or (b) the first promotor sequence comprises SEQ ID NO: 17, the first termination sequence comprises SEQ ID NO: 1265, the second promotor sequence comprises SEQ ID NO: 1262 and the second termination sequence comprises SEQ ID NO: 1264.

In various aspects, the present disclosure provides a viral vector encapsidating the expression cassette as described herein or the recombinant polynucleotide as described herein.

In some aspects, the viral vector comprises two or more, three or more, or four or more expression cassettes as described herein. In some aspects, the viral vector is an adeno-associated viral vector. In some aspects, the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, chimeras thereof, and combinations thereof.

In various aspects, the present disclosure provides a pharmaceutical composition comprising the expression cassette as described herein, the recombinant polynucleotide as described herein, or the viral vector as described herein and a pharmaceutically acceptable excipient, carrier, diluent, or combination thereof.

In various aspects, the present disclosure provides a method of expressing a small RNA payload in a cell, the method comprising delivering the expression cassette as described herein, the recombinant polynucleotide as described herein, the viral vector as described herein, or the pharmaceutical composition as described herein to a cell and expressing the small RNA payload encoded by the expression cassette in the cell.

In various aspects, the present disclosure provides a method of editing a target sequence, the method comprising: delivering the expression cassette as described herein, the recombinant polynucleotide as described herein, the viral vector as described herein, or the pharmaceutical composition as described herein to a cell encoding the target sequence; expressing the small RNA payload in the cell, wherein the small RNA payload comprises an engineered guide RNA capable of hybridizing to a target sequence; forming a guide-target RNA scaffold upon hybridization of the small RNA payload to the target sequence; recruiting an editing enzyme to the target sequence; and editing the target sequence with the editing enzyme.

In various aspects, the present disclosure provides a method of editing a target sequence, the method comprising: delivering an expression cassette to a cell encoding the target sequence, wherein the expression cassette comprises: a promoter sequence comprising a sequence having at least 80% sequence identity to any one of: a) SEQ ID NO: 17, SEQ ID NO: 1250, or SEQ ID NO: 1262; b) SEQ ID NO: 13 or SEQ ID NO: 15; or c) SEQ ID NO: 1241, SEQ ID NO: 1251, SEQ ID NO: 1252, SEQ ID NO: 1253, or SEQ ID NO: 1263; a payload sequence under transcriptional control of the promoter sequence, the payload sequence comprising a small RNA payload; and a termination sequence comprising a sequence having at least 80% identity to any one of: a) SEQ ID NO: 1002, SEQ ID NO: 1017, SEQ ID NO: 1264, or SEQ ID NO: 1265; or b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269; expressing the small RNA payload in the cell; forming a guide-target RNA scaffold upon hybridization of the small RNA payload to the target sequence; recruiting an editing enzyme to the target sequence; and editing the target sequence with the editing enzyme.

In various aspects, the present disclosure provides a method of editing a target sequence, the method comprising: delivering an expression cassette to a cell encoding the target sequence, wherein the expression cassette comprises: a promoter sequence comprising a sequence having at least 80% sequence identity to any one of: a) SEQ ID NO: 17, SEQ ID NO: 1250, or SEQ ID NO: 1262; b) SEQ ID NO: 13 or SEQ ID NO: 15; or c) SEQ ID NO: 1241, SEQ ID NO: 1251, SEQ ID NO: 1252, SEQ ID NO: 1253, or SEQ ID NO: 1263; a payload sequence under transcriptional control of the promoter sequence, the payload sequence comprising a small RNA payload; and a termination sequence; expressing the small RNA payload in the cell; forming a guide-target RNA scaffold upon hybridization of the small RNA payload to the target sequence; recruiting an editing enzyme to the target sequence; and editing the target sequence with the editing enzyme.

In various aspects, the present disclosure provides a method of editing a target sequence, the method comprising: delivering an expression cassette to a cell encoding the target sequence, wherein the expression cassette comprises: a promoter sequence; a payload sequence under transcriptional control of the promoter sequence, the payload sequence comprising a small RNA payload; and a termination sequence comprising a sequence having at least 80% identity to any one of: a) SEQ ID NO: 1002, SEQ ID NO: 1017, SEQ ID NO: 1264, or SEQ ID NO: 1265; or b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269; expressing the small RNA payload in the cell; forming a guide-target RNA scaffold upon hybridization of the small RNA payload to the target sequence; recruiting an editing enzyme to the target sequence; and editing the target sequence with the editing enzyme.

In various aspects, the present disclosure provides a method of editing a target sequence, the method comprising: delivering an expression cassette to a cell encoding the target sequence, wherein the expression cassette comprises: a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263; a payload sequence under transcriptional control of the promoter sequence, the payload sequence comprising a small RNA payload; and a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289; expressing the small RNA payload in the cell; forming a guide-target RNA scaffold upon hybridization of the small RNA payload to the target sequence; recruiting an editing enzyme to the target sequence; and editing the target sequence with the editing enzyme.

In various aspects, the present disclosure provides a method of editing a target sequence, the method comprising: delivering an expression cassette to a cell encoding the target sequence, wherein the expression cassette comprises: a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263; a payload sequence under transcriptional control of the promoter sequence, the payload sequence comprising a small RNA payload; and a termination sequence; expressing the small RNA payload in the cell; forming a guide-target RNA scaffold upon hybridization of the small RNA payload to the target sequence; recruiting an editing enzyme to the target sequence; and editing the target sequence with the editing enzyme.

In various aspects, the present disclosure provides a method of editing a target sequence, the method comprising: delivering an expression cassette to a cell encoding the target sequence, wherein the expression cassette comprises: a promoter sequence; a payload sequence under transcriptional control of the promoter sequence, the payload sequence comprising a small RNA payload; and a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289; expressing the small RNA payload in the cell; forming a guide-target RNA scaffold upon hybridization of the small RNA payload to the target sequence; recruiting an editing enzyme to the target sequence; and editing the target sequence with the editing enzyme.

In some aspects, the promoter sequence comprises SEQ ID NO: 17. In some aspects, the promoter sequence comprises SEQ ID NO: 1262. In some aspects, the promoter sequence comprises SEQ ID NO: 1250. In some aspects, the promoter sequence comprises SEQ ID NO: 1251. In some aspects, the promoter sequence comprises SEQ ID NO: 1252. In some aspects, the promoter sequence comprises SEQ ID NO: 1253.

In some aspects, the termination sequence comprises SEQ ID NO: 1264. In some aspects, the termination sequence comprises SEQ ID NO: 1265. In some aspects, the termination sequence comprises SEQ ID NO: 1254. In some aspects, the termination sequence comprises SEQ ID NO: 1255. In some aspects, the termination sequence comprises SEQ ID NO: 1257. In some aspects, the termination sequence comprises SEQ ID NO: 60. In some aspects, the termination sequence comprises SEQ ID NO: 1242. In some aspects, the termination sequence comprises SEQ ID NO: 1269. In some aspects, the termination sequence comprises SEQ ID NO: 1017.

In some aspects, the target sequence comprises a mutation relative to a wild type sequence. In some aspects, editing the target sequence corrects the mutation in the target sequence. In some aspects, the mutation is a missense mutation. In some aspects, the mutation is a nonsense mutation. In some aspects, the mutation is a G to A mutation. In some aspects, the mutation is associated with a disease. In some aspects, the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease.

In some aspects, the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2).

In some aspects, editing the target sequence comprises editing an untranslated region of the target. In some aspects, the untranslated region is a 5′ untranslated region or a 3′ untranslated region. In some aspects, the 3′ untranslated region is a polyadenylation sequence. In some aspects, editing the target sequence comprises editing a translation initiation site.

In some aspects, editing the target sequence alters expression of the target sequence. In some aspects, editing the target sequence increases expression of the target sequence. In some aspects, editing the target sequence decreases expression of the target sequence.

In various aspects, the present disclosure provides a method of treating a disease in a subject, the method comprising: administering to the subject a composition comprising the expression cassette as described herein, the recombinant polynucleotide as described herein, the viral vector as described herein, or the pharmaceutical composition as described herein; delivering the expression cassette to a cell of the subject; and expressing the small RNA payload in the cell, thereby treating the disease.

In various aspects, the present disclosure provides a method of treating a disease in a subject, the method comprising: administering to the subject a composition comprising an expression cassette comprising: a promoter sequence comprising a sequence having at least 80% sequence identity to any one of: a) SEQ ID NO: 17, SEQ ID NO: 1250, or SEQ ID NO: 1262; b) SEQ ID NO: 13 or SEQ ID NO: 15; or c) SEQ ID NO: 1241, SEQ ID NO: 1251, SEQ ID NO: 1252, SEQ ID NO: 1253, or SEQ ID NO: 1263; a payload sequence under transcriptional control of the promoter sequence, the payload sequence comprising a small RNA payload; and a termination sequence comprising a sequence having at least 80% identity to any one of: a) SEQ ID NO: 1002, SEQ ID NO: 1017, SEQ ID NO: 1264, or SEQ ID NO: 1265; or b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269; delivering the expression cassette to a cell of the subject; and expressing the small RNA payload in the cell, thereby treating the disease.

In various aspects, the present disclosure provides a method of treating a disease in a subject, the method comprising: administering to the subject a composition comprising an expression cassette comprising: a promoter sequence comprising a sequence having at least 80% sequence identity to any one of: a) SEQ ID NO: 17, SEQ ID NO: 1250, or SEQ ID NO: 1262; b) SEQ ID NO: 13 or SEQ ID NO: 15; or c) SEQ ID NO: 1241, SEQ ID NO: 1251, SEQ ID NO: 1252, SEQ ID NO: 1253 or SEQ ID NO: 1263; a payload sequence under transcriptional control of the promoter sequence, the payload sequence comprising a small RNA payload; and a termination sequence; delivering the expression cassette to a cell of the subject; and expressing the small RNA payload in the cell, thereby treating the disease.

In various aspects, the present disclosure provides a method of treating a disease in a subject, the method comprising: administering to the subject a composition comprising an expression cassette comprising: a promoter sequence; a payload sequence under transcriptional control of the promoter sequence, the payload sequence comprising a small RNA payload; and a termination sequence comprising a sequence having at least 80% identity to any one of: a) SEQ ID NO: 1002, SEQ ID NO: 1017, SEQ ID NO: 1264, or SEQ ID NO: 1265; or b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269; delivering the expression cassette to a cell of the subject; and expressing the small RNA payload in the cell, thereby treating the disease.

In various aspects, the present disclosure provides a method of treating a disease in a subject, the method comprising: administering to the subject a composition comprising an expression cassette comprising: a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263; a payload sequence under transcriptional control of the promoter sequence, the payload sequence comprising a small RNA payload; and a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289; delivering the expression cassette to a cell of the subject; and expressing the small RNA payload in the cell, thereby treating the disease.

In various aspects, the present disclosure provides a method of treating a disease in a subject, the method comprising: administering to the subject a composition comprising an expression cassette comprising: a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263; a payload sequence under transcriptional control of the promoter sequence, the payload sequence comprising a small RNA payload; and a termination sequence; delivering the expression cassette to a cell of the subject; and expressing the small RNA payload in the cell, thereby treating the disease.

In various aspects, the present disclosure provides a method of treating a disease in a subject, the method comprising: administering to the subject a composition comprising an expression cassette comprising: a promoter sequence; a payload sequence under transcriptional control of the promoter sequence, the payload sequence comprising a small RNA payload; and a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289; delivering the expression cassette to a cell of the subject; and expressing the small RNA payload in the cell, thereby treating the disease.

In some aspects, the promoter sequence comprises SEQ ID NO: 17. In some aspects, the promoter sequence comprises SEQ ID NO: 1262. In some aspects, the promoter sequence comprises SEQ ID NO: 1250. In some aspects, the promoter sequence comprises SEQ ID NO: 1251. In some aspects, the promoter sequence comprises SEQ ID NO: 1252. In some aspects, the promoter sequence comprises SEQ ID NO: 1253.

In some aspects, the termination sequence comprises SEQ ID NO: 1264. In some aspects, the termination sequence comprises SEQ ID NO: 1265. In some aspects, the termination sequence comprises SEQ ID NO: 1254. In some aspects, the termination sequence comprises SEQ ID NO: 1255. In some aspects, the termination sequence comprises SEQ ID NO: 1257. In some aspects, the termination sequence comprises SEQ ID NO: 60. In some aspects, the termination sequence comprises SEQ ID NO: 1242. In some aspects, the termination sequence comprises SEQ ID NO: 1269. In some aspects, the termination sequence comprises SEQ ID NO: 1017.

In some aspects, the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease.

In some aspects, the small RNA payload comprises an engineered guide RNA that hybridizes to a target sequence, and wherein the cell encodes the target sequence. In some aspects, the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2).

In some aspects, the method further comprises forming a guide-target RNA scaffold upon hybridization of the engineered guide RNA to the target sequence, recruiting an editing enzyme to the target sequence, and editing the target sequence with the editing enzyme. In some aspects, the target sequence comprises a mutation relative to a wild type sequence. In some aspects, editing the target sequence corrects the mutation in the target sequence. In some aspects, the mutation is a missense mutation. In some aspects, the mutation is a nonsense mutation. In some aspects, the mutation is a G to A mutation. In some aspects, the mutation is associated with the disease. In some aspects, editing the target sequence comprises editing an untranslated region of the target. In some aspects, the untranslated region is a 5′ untranslated region or a 3′ untranslated region. In some aspects, the 3′ untranslated region is a polyadenylation sequence. In some aspects, editing the target sequence comprises editing a translation initiation site.

In some aspects, editing the target sequence alters expression of the target sequence. In some aspects, editing the target sequence increases expression of the target sequence. In some aspects, editing the target sequence decreases expression of the target sequence.

In some aspects, the guide-target RNA scaffold comprises a structural feature. In some aspects, the structural feature is a bulge, a mismatch, an internal loop, a hairpin, or combinations thereof. In some aspects, the structural feature comprises the bulge, and wherein the bulge is a symmetric bulge. In some aspects, the structural feature comprises the bulge, and wherein the bulge is an asymmetric bulge. In some aspects, the structural feature comprises the internal loop, and wherein the internal loop is a symmetric internal loop. In some aspects, the structural feature comprises the internal loop, and wherein the internal loop is an asymmetric internal loop. In some aspects, the structural feature comprises the hairpin, and wherein the hairpin is a recruitment hairpin or a non-recruitment hairpin. In some aspects, the guide-target RNA scaffold comprises a Wobble base pair.

In some aspects, the editing enzyme comprises an ADAR, an APOBEC, or a Cas nuclease. In some aspects, the ADAR comprises ADAR1, ADAR2, ADAR3, or combinations thereof. In some aspects, the target sequence comprises RNA or DNA. In some aspects, the target sequence is a mRNA or a pre-mRNA. In some aspects, editing the target sequence comprises deamidating a nucleotide of the target sequence. In some aspects, the target sequence is edited with an efficiency of at least 10%, at least 20%, or at least 25%.

In various aspects, the present disclosure provides an expression cassette comprising: a promoter sequence comprising: a zinc finger 143 motif, an OCT-1 transcription factor binding sequence, a proximal sequence element; a payload sequence under transcriptional control of the promoter sequence, the payload sequence comprising a small RNA payload; and a transcription termination sequence; wherein the expression cassette comprises one or more sequence elements selected from the group consisting of: a) the zinc finger 143 motif having at least 80% sequence identity to any one of SEQ ID NO: 24-SEQ ID NO: 26, b) the OCT-1 transcription factor binding sequence having at least 80% sequence identity to any one of SEQ ID NO: 27-SEQ ID NO: 30, c) the proximal sequence element having at least 80% sequence identity to any one of SEQ ID NO: 31-SEQ ID NO: 37, and d) combinations thereof.

In some aspects, the zinc finger 143 motif comprises at least 80%, at least 85%, at least 90%, at least 95%, or 100% sequence identity to any one of SEQ ID NO: 24-SEQ ID NO: 26. In some aspects, the zinc finger 143 motif comprises at least 80%, at least 85%, at least 90%, at least 95%, or 100% sequence identity to SEQ ID NO: 20. In some aspects, the OCT-1 transcription factor binding sequence comprises at least 80%, at least 85%, at least 90%, at least 95%, or 100% sequence identity to any one of SEQ ID NO: 27-SEQ ID NO: 30. In some aspects, the proximal sequence element comprises at least 80%, at least 85%, at least 90%, at least 95%, or 100% sequence identity to any one of SEQ ID NO: 31-SEQ ID NO: 37.

In some aspects, the transcription termination sequence comprises at least 80%, at least 85%, at least 90%, at least 95%, or 100% sequence identity to any one of SEQ ID NO: 40-SEQ ID NO: 42. In some aspects, the transcription termination sequence comprises at least 80%, at least 85%, at least 90%, at least 95%, or 100% sequence identity to SEQ ID NO: 60, SEQ ID NO: 1242-SEQ ID NO: 1247, or SEQ ID NO: 1254-SEQ ID NO: 1257. In some aspects, the transcription termination sequence comprises at least 80%, at least 85%, at least 90%, at least 95%, or 100% sequence identity to SEQ ID NO: 1242. In some aspects, the transcription termination sequence comprises a sequence of SEQ ID NO: 1242. In some aspects, the transcription termination sequence comprises at least 80%, at least 85%, at least 90%, at least 95%, or 100% sequence identity to SEQ ID NO: 60. In some aspects, the transcription termination sequence comprises a sequence of SEQ ID NO: 60. In some aspects, the transcription termination sequence comprises a sequence of SEQ ID NO: 38 or SEQ ID NO: 39.

In various aspects, the present disclosure provides an expression cassette comprising: a promoter sequence comprising a proximal sequence element, wherein the promoter sequence comprises a sequence having at least 75% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253 in which the proximal sequence element of the promoter sequence is replaced with a sequence of any one of SEQ ID NO: 67-SEQ ID NO: 120; a payload sequence under transcriptional control of the promoter sequence, the payload sequence comprising a small RNA payload; and a transcription termination sequence comprising a 3′ box sequence element, wherein the transcription termination sequence comprises a sequence having at least 75% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257 in which the 3′ box sequence element of the termination sequence is replaced with a sequence of any one of SEQ ID NO: 121-SEQ ID NO: 166.

In some aspects, the promoter sequence comprises a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253 in which the proximal sequence element of the promoter sequence is replaced with a sequence of any one of SEQ ID NO: 67-SEQ ID NO: 120. In some aspects, the promoter sequence comprises a sequence having at least 90% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253 in which the proximal sequence element of the promoter sequence is replaced with a sequence of any one of SEQ ID NO: 67-SEQ ID NO: 120. In some aspects, the termination sequence comprises a sequence having at least 80% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257 in which the 3′ box sequence element of the termination sequence is replaced with a sequence of any one of SEQ ID NO: 121-SEQ ID NO: 166. In some aspects, the termination sequence comprises a sequence having at least 90% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257 in which the 3′ box sequence element of the termination sequence is replaced with a sequence of any one of SEQ ID NO: 121-SEQ ID NO: 166.

In various aspects, the present disclosure provides an expression cassette comprising a promoter sequence comprising a sequence having at least 75% sequence identity to any one of SEQ ID NO: 16-SEQ ID NO: 17, SEQ ID NO: 167-SEQ ID NO: 707, SEQ ID NO: 1241, SEQ ID NO: 1248-SEQ ID NO: 1253; a payload sequence under transcriptional control of the promoter sequence, the payload sequence comprising a small RNA payload; and a transcription termination sequence comprising a sequence having at least 75% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257.

In some aspects, the promoter sequence comprises a sequence having at least 80% sequence identity to any one of SEQ ID NO: 16-SEQ ID NO: 17, SEQ ID NO: 167-SEQ ID NO: 707, SEQ ID NO: 1241, SEQ ID NO: 1248-SEQ ID NO: 1253. In some aspects, the promoter sequence comprises a sequence having at least 90% sequence identity to any one of SEQ ID NO: 16-SEQ ID NO: 17, SEQ ID NO: 167-SEQ ID NO: 707, SEQ ID NO: 1241, SEQ ID NO: 1248-SEQ ID NO: 1253. In some aspects, the termination sequence comprises a sequence having at least 80% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257. In some aspects, the termination sequence comprises a sequence having at least 90% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257.

In some aspects, the promoter sequence is SEQ ID NO: 376. In some aspects, the promoter sequence is SEQ ID NO: 1250. In some aspects, the transcription termination sequence is SEQ ID NO: 917. In some aspects, the transcription termination sequence is SEQ ID NO: 1254. In some aspects, the promoter sequence is SEQ ID NO: 168. In some aspects, the promoter sequence is SEQ ID NO: 1251. In some aspects, the transcription termination sequence is SEQ ID NO: 709. In some aspects, the transcription termination sequence is SEQ ID NO: 1255. In some aspects, the promoter sequence is SEQ ID NO: 1241. In some aspects, the transcription termination sequence is SEQ ID NO: 1242 or SEQ ID NO: 60. In some aspects, the promoter sequence is SEQ ID NO: 17. In some aspects, the transcription termination sequence is SEQ ID NO: 1242 or SEQ ID NO: 60.

In some aspects, the small RNA payload comprises an engineered guide RNA capable of hybridizing to a target sequence.

In some aspects, the engineered guide RNA is at least 70%, at least 75%, at least 80%, at least 85%, at least 90%, at least 95%, or 100% reverse complementary to the target sequence. In some aspects, the engineered guide RNA comprises at least one base pair mismatch relative to the target sequence. In some aspects, the target sequence comprises an adenosine residue. In some aspects, the target sequence is an RNA sequence. In some aspects, the RNA sequence is a mRNA or a pre-mRNA.

In some aspects, the target sequence comprises a G to A mutation relative to a wild type sequence. In some aspects, the target sequence comprises a missense mutation or a nonsense mutation relative to a wild type sequence. In some aspects, the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2). In some aspects, the payload sequence comprises at least 80%, at least 85%, at least 90%, at least 95%, or 100% sequence identity to SEQ ID NO: 1273, SEQ ID NO: 1274, or SEQ ID NO: 61.

In some aspects, the small RNA payload comprises an antisense oligonucleotide, an siRNA, an shRNA, a miRNA, or a tracrRNA. In some aspects, the small RNA payload is not less than 20 nucleotide residues and not more than 500 nucleotide residues long. In some aspects, the small RNA payload is not less than 60 and not more than 100 residues long. In some aspects, the small RNA payload is not less than 80 and not more than 120 residues long. In some aspects, the small RNA payload is not less than 100 and not more than 140 residues long. In some aspects, the small RNA payload is not less than 130 and not more than 170 residues long.

In some aspects, the payload sequence further comprises an Sm binding sequence or a hairpin sequence. In some aspects, the hairpin sequence comprises a U7 hairpin. In some aspects, the hairpin sequence comprises at least 80%, at least 85%, at least 90%, at least 95%, or 100% sequence identity to SEQ ID NO: 52, SEQ ID NO: 54, SEQ ID NO: 56, or SEQ ID NO: 58.

In some aspects, the expression cassette comprises two or more of the sequence elements. In some aspects, the expression cassette comprises three or more of the sequence elements. In some aspects, the expression cassette has a length of not less than 1300 nucleotide residues and not more than 2160 nucleotide residues. In some aspects, the expression cassette comprises at least 80% sequence identity to a U1 sequence or a U7 sequence. In some aspects, the U1 sequence is a mouse U1 sequence or a human U1 sequence. In some aspects, the U7 sequence is a mouse U7 sequence or a human U7 sequence.

In some aspects, the promoter sequence comprises at least 80%, at least 85%, at least 90%, at least 95%, or 100% sequence identity to any one of SEQ ID NO: 13-SEQ ID NO: 17, SEQ ID NO: 1241, SEQ ID NO: 1248-SEQ ID NO: 1253. In some aspects, the promoter sequence comprises at least 80%, at least 85%, at least 90%, at least 95%, or 100% sequence identity to SEQ ID NO: 1241. In some aspects, the promoter sequence comprises a sequence of SEQ ID NO: 1241. In some aspects, the transcription termination sequence is SEQ ID NO: 1242 or SEQ ID NO: 60. In some aspects, the promoter sequence comprises at least 80%, at least 85%, at least 90%, at least 95%, or 100% sequence identity to SEQ ID NO: 17. In some aspects, the promoter sequence comprises a sequence of SEQ ID NO: 17. In some aspects, the transcription termination sequence is SEQ ID NO: 1242 or SEQ ID NO: 60.

In some aspects, the expression cassette comprises at least 80%, at least 85%, at least 90%, at least 95%, or 100% sequence identity to any one of SEQ ID NO: 1-SEQ ID NO: 12 or SEQ ID NO: 59. In some aspects, the zinc finger 143 motif is capable of recruiting a ZNF143 transcription factor. In some aspects, the OCT-1 transcription factor binding sequence is capable of recruiting an OCT-1 transcription factor. In some aspects, the proximal sequence element is capable of recruiting a SNAPc. In some aspects, the proximal sequence element is capable of integrator dependent recruitment of RNA polymerase II.

In some aspects, the small RNA payload is capable of forming a guide-target RNA scaffold comprising a structural feature upon hybridization of the small RNA payload to a target sequence. In some aspects, the structural feature is a bulge, a mismatch, an internal loop, a hairpin, or combinations thereof. In some aspects, the structural feature comprises the bulge, and wherein the bulge is a symmetric bulge. In some aspects, the structural feature comprises the bulge, and wherein the bulge is an asymmetric bulge. In some aspects, the structural feature comprises the internal loop, and wherein the internal loop is a symmetric internal loop. In some aspects, the structural feature comprises the internal loop, and wherein the internal loop is an asymmetric internal loop. In some aspects, the structural feature comprises the hairpin, and wherein the hairpin is a recruitment hairpin or a non-recruitment hairpin. the guide-target RNA scaffold comprises a Wobble base pair.

In various aspects, the present disclosure provides a method of expressing a small RNA payload in a cell, the method comprising delivering an expression cassette as described herein to a cell and expressing the small RNA payload encoded by the expression cassette in the cell.

In various aspects, the present disclosure provides a method of editing a target sequence, the method comprising: delivering an expression cassette to a cell encoding the target sequence, wherein the expression cassette comprises: a promoter sequence comprising: a zinc finger 143 motif, an OCT-1 transcription factor binding sequence, and a proximal sequence element, a payload sequence under transcriptional control of the promoter sequence, the payload sequence comprising a small RNA payload, wherein the small RNA payload comprises an engineered guide RNA sequence capable of hybridizing to the target sequence, and a transcription termination sequence; expressing the small RNA payload in the cell; forming a guide-target RNA scaffold upon hybridization of the small RNA payload to the target sequence; recruiting an editing enzyme to the target sequence; and editing the target sequence with the editing enzyme.

In various aspects, the present disclosure provides a method of editing a target sequence, the method comprising: delivering an expression cassette to a cell encoding the target sequence, wherein the expression cassette comprises: a promoter sequence comprising a proximal sequence element, wherein the promoter sequence comprises a sequence having at least 75% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253 in which the proximal sequence element of the promoter sequence is replaced with a sequence of any one of SEQ ID NO: 67-SEQ ID NO: 120; a payload sequence under transcriptional control of the promoter sequence, the payload sequence comprising a small RNA payload; and a transcription termination sequence comprising a 3′ box sequence element, wherein the transcription termination sequence comprises a sequence having at least 75% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257 in which the 3′ box sequence element of the termination sequence is replaced with a sequence of any one of SEQ ID NO: 121-SEQ ID NO: 166; expressing the small RNA payload in the cell; forming a guide-target RNA scaffold upon hybridization of the small RNA payload to the target sequence; recruiting an editing enzyme to the target sequence; and editing the target sequence with the editing enzyme.

In various aspects, the present disclosure provides a method of editing a target sequence, the method comprising: delivering an expression cassette to a cell encoding the target sequence, wherein the expression cassette comprises: a promoter sequence comprising a proximal sequence element, wherein the promoter sequence comprises a sequence having at least 75% sequence identity to any one of SEQ ID NO: 16-SEQ ID NO: 17, SEQ ID NO: 167-SEQ ID NO: 707, SEQ ID NO: 1241, SEQ ID NO: 1248-SEQ ID NO: 1253; a payload sequence under transcriptional control of the promoter sequence, the payload sequence comprising a small RNA payload; and a transcription termination sequence comprising a 3′ box sequence element, wherein the transcription termination sequence comprises a sequence having at least 75% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257; expressing the small RNA payload in the cell; forming a guide-target RNA scaffold upon hybridization of the small RNA payload to the target sequence; recruiting an editing enzyme to the target sequence; and editing the target sequence with the editing enzyme.

In various aspects, the promoter sequence is SEQ ID NO: 376. In various aspects, the promoter sequence is SEQ ID NO: 1250. In various aspects, the transcription termination sequence is SEQ ID NO: 917. In various aspects, the transcription termination sequence is SEQ ID NO: 1254. In various aspects, the promoter sequence is SEQ ID NO: 168. In various aspects, the promoter sequence is SEQ ID NO: 1251. In various aspects, transcription termination sequence is SEQ ID NO: 709. In various aspects, the transcription termination sequence is SEQ ID NO: 1255. In various aspects, the promoter sequence is SEQ ID NO: 1241. In various aspects, the transcription termination sequence is SEQ ID NO: 1242 or SEQ ID NO: 60. In various aspects, the promoter sequence is SEQ ID NO: 17. In various aspects, the transcription termination sequence is SEQ ID NO: 1242 or SEQ ID NO: 60.

In various aspects, the present disclosure provides a method of editing a target sequence, the method comprising: delivering the expression cassette as described herein to a cell encoding the target sequence; expressing the small RNA payload in the cell, wherein the small RNA payload comprises an engineered guide RNA capable of hybridizing to a target sequence; forming a guide-target RNA scaffold upon hybridization of the small RNA payload to the target sequence; recruiting an editing enzyme to the target sequence; and editing the target sequence with the editing enzyme.

In some aspects, the target sequence comprises a mutation relative to a wild type sequence. In some aspects, editing the target sequence corrects the mutation in the target sequence. In some aspects, the mutation is a missense mutation. In some aspects, the mutation is a nonsense mutation. In some aspects, the mutation is a G to A mutation. In some aspects, the mutation is associated with a disease.

In some aspects, the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease. In some aspects, the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2).

In some aspects, editing the target sequence comprises editing an untranslated region of the target. In some aspects, the untranslated region is a 5′ untranslated region or a 3′ untranslated region. In some aspects, the 3′ untranslated region is a polyadenylation sequence. In some aspects, editing the target sequence comprises editing a translation initiation site. In some aspects, editing the target sequence alters expression of the target sequence. In some aspects, editing the target sequence increases expression of the target sequence. In some aspects, editing the target sequence decreases expression of the target sequence.

In various aspects, the present disclosure provides a method of treating a disease in a subject, the method comprising: administering to the subject a composition comprising an expression cassette comprising: a promoter sequence comprising: a zinc finger 143 motif, an OCT-1 transcription factor binding sequence, and a proximal sequence element, and a payload sequence under transcriptional control of the promoter sequence, the payload sequence comprising a small RNA payload; delivering the expression cassette to a cell of the subject; and expressing the small RNA payload in the cell, thereby treating the disease.

In various aspects, the present disclosure provides a method of treating a disease in a subject, the method comprising: administering to the subject a composition comprising an expression cassette comprising: a promoter sequence comprising a proximal sequence element, wherein the promoter sequence comprises a sequence having at least 75% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253 in which the proximal sequence element of the promoter sequence is replaced with a sequence of any one of SEQ ID NO: 67-SEQ ID NO: 120; a payload sequence under transcriptional control of the promoter sequence, the payload sequence comprising a small RNA payload; and a transcription termination sequence comprising a 3′ box sequence element, wherein the transcription termination sequence comprises a sequence having at least 75% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257 in which the 3′ box sequence element of the termination sequence is replaced with a sequence of any one of SEQ ID NO: 121-SEQ ID NO: 166; delivering the expression cassette to a cell of the subject; and expressing the small RNA payload in the cell, thereby treating the disease.

In various aspects, the present disclosure provides a method of treating a disease in a subject, the method comprising: administering to the subject a composition comprising an expression cassette comprising: a promoter sequence comprising a proximal sequence element, wherein the promoter sequence comprises a sequence having at least 75% sequence identity to any one of SEQ ID NO: 16-SEQ ID NO: 17, SEQ ID NO: 167-SEQ ID NO: 707, SEQ ID NO: 1241, SEQ ID NO: 1248-SEQ ID NO: 1253; a payload sequence under transcriptional control of the promoter sequence, the payload sequence comprising a small RNA payload; and a transcription termination sequence comprising a 3′ box sequence element, wherein the transcription termination sequence comprises a sequence having at least 75% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257; delivering the expression cassette to a cell of the subject; and expressing the small RNA payload in the cell, thereby treating the disease.

In some aspects, the promoter sequence is SEQ ID NO: 376. In some aspects, the promoter sequence is SEQ ID NO: 1250. In some aspects, the transcription termination sequence is SEQ ID NO: 917. In some aspects, the transcription termination sequence is SEQ ID NO: 1254. In some aspects, the promoter sequence is SEQ ID NO: 168. In some aspects, the promoter sequence is SEQ ID NO: 1251. In some aspects, the transcription termination sequence is SEQ ID NO: 709. In some aspects, the transcription termination sequence is SEQ ID NO: 1255. In some aspects, the promoter sequence is SEQ ID NO: 1241. In some aspects, the transcription termination sequence is SEQ ID NO: 1242 or SEQ ID NO: 60. In some aspects, the promoter sequence is SEQ ID NO: 17. In some aspects, the transcription termination sequence is SEQ ID NO: 1242 or SEQ ID NO: 60.

In various aspects, the present disclosure provides a method of treating a disease in a subject, the method comprising: administering to the subject a composition comprising an expression cassette as described herein; delivering the expression cassette to a cell of the subject; and expressing a small RNA payload in the cell, thereby treating the disease.

In some aspects, the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease. In some aspects, the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2). In some aspects, the small RNA payload comprises an engineered guide RNA that hybridizes to a target sequence, and wherein the cell encodes the target sequence.

In some aspects, the method further comprises forming a guide-target RNA scaffold upon hybridization of the engineered guide RNA to the target sequence, recruiting an editing enzyme to the target sequence, and editing the target sequence with the editing enzyme. In some aspects, the target sequence comprises a mutation relative to a wild type sequence. In some aspects, editing the target sequence corrects the mutation in the target sequence. In some aspects, the mutation is a missense mutation. In some aspects, the mutation is a nonsense mutation. In some aspects, the mutation is a G to A mutation. In some aspects, the mutation is associated with the disease.

In some aspects, editing the target sequence comprises editing an untranslated region of the target. In some aspects, the untranslated region is a 5′ untranslated region or a 3′ untranslated region. In some aspects, the 3′ untranslated region is a polyadenylation sequence. In some aspects, editing the target sequence comprises editing a translation initiation site. In some aspects, editing the target sequence alters expression of the target sequence. In some aspects, editing the target sequence increases expression of the target sequence. In some aspects, editing the target sequence decreases expression of the target sequence.

In some aspects, the guide-target RNA scaffold comprises a structural feature. In some aspects, the structural feature is a bulge, a mismatch, an internal loop, a hairpin, or combinations thereof. In some aspects, the structural feature comprises the bulge, and wherein the bulge is a symmetric bulge. In some aspects, the structural feature comprises the bulge, and wherein the bulge is an asymmetric bulge. In some aspects, the structural feature comprises the internal loop, and wherein the internal loop is a symmetric internal loop. In some aspects, the structural feature comprises the internal loop, and wherein the internal loop is an asymmetric internal loop. In some aspects, the structural feature comprises the hairpin, and wherein the hairpin is a recruitment hairpin or a non-recruitment hairpin. In some aspects, the guide-target RNA scaffold comprises a Wobble base pair.

In some aspects, the editing enzyme comprises an ADAR, an APOBEC, or a Cas nuclease. In some aspects, the ADAR comprises ADAR1, ADAR2, ADAR3, or combinations thereof. In some aspects, the target sequence comprises RNA or DNA. In some aspects, the target sequence is a mRNA or a pre-mRNA. In some aspects, editing the target sequence comprises deamidating a nucleotide of the target sequence. In some aspects, the target sequence is edited with an efficiency of at least 10%, at least 20%, or at least 25%.

In some aspects, the expression cassette is delivered to the cell via a viral vector. In some aspects, the viral vector is an adenoviral vector, an adeno-associated viral vector, or a lentivector. In some aspects, the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, chimeras thereof, and combinations thereof.

In various aspects, the present disclosure provides a viral vector encapsidating an expression cassette as described herein.

In some aspects, the viral vector is an adeno-associated viral vector. In some aspects, the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, chimeras thereof, and combinations thereof.

In various aspects, the present disclosure provides a pharmaceutical composition comprising an expression cassette as described herein or a viral vector as described herein and a pharmaceutically acceptable excipient, carrier, diluent, or combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A schematically illustrates an example configuration of an engineered guide RNA expression cassette based on a mouse U7 (mU7) promoter. The expression cassette encodes a payload sequence under transcriptional control of a mU7 promoter. The mU7 promoter includes an SPH element (e.g., a zinc finger 143 motif), an OCT-1 transcription factor binding sequence, and a proximal sequence element (PSE). The payload sequence, which begins at the transcriptional start site and ends at the termination sequence, includes an engineered guide RNA sequence (“guide”) operably linked to an Sm binding sequence (smOPT).

FIG. 1B schematically illustrates an example configuration of an engineered guide RNA expression cassette based on a human U1 (hU1) promoter. The expression cassette encodes a payload sequence under transcriptional control of an hU1 promoter. The hU1promoter includes an SPH element (e.g., a zinc finger 143 motif), an OCT-1 transcription factor binding sequence, and a proximal sequence element (PSE). The payload sequence, which begins at the transcriptional start site and ends at the termination sequence, includes an engineered guide RNA sequence (“guide”) operably linked to an Sm binding sequence (smOPT).

FIG. 2A schematically illustrates a reporter construct for measuring expression of an engineered guide RNA sequence and subsequent editing of the target RNA sequence. The report construct includes a target sequence (e.g., CDS1) containing an ATG start site that can be edited to ITG, read as GTG, by ADAR-catalyzed deamidation. Conversion of ATG to GTG results in an increase in luciferase (NanoLuc) expression.

FIG. 2B shows a bar plot of a luciferase assay demonstrating editing of a reporter construct by an engineered guide RNA construct. The unedited (ATG) construct expresses basal levels of luciferase, resulting in background levels of luciferase activity. The edited (GTG) construct expresses higher levels of luciferase, resulting in elevated luciferase activity relative to that of the unedited construct.

FIG. 3 shows a bar plot of a luciferase activity in the presence of unedited (A) or edited (G) reporters of SEQ ID NO: 48 (“fPMP22-cDNA (ATG)”), SEQ ID NO: 49 (“fSNCA-pre (ATG)”), and SEQ ID NO: 50 (“fSNCA-cDNA (ATG)”). For each reporter, the edited construct expressed higher levels of luciferase, resulting in increased levels of luciferase activity, relative to the unedited constructs.

FIG. 4 schematically illustrates a workflow for generating and evaluating expression of an expression cassette constructs. Cells are transfected with engineered guide RNA-encoding plasmids, and engineered guide RNA expression is evaluated by luciferase activity. Expression of the engineered guide RNA can be further evaluated using mirVANA total RNA isolation, DNaseI treatment, ddPCR guide quantification assays, or Sanger editing.

FIG. 5 shows a bar plot of a luciferase assay to evaluate expression of an SNCA-targeting engineered guide RNA (SEQ ID NO: 1274) under control of a mouse U7 promoter with various OCT-1 transcription factor binding sequences. The original OCT-1 transcription factor binding sequence (SEQ ID NO: 21) in the SNCA-targeting guide RNA expression cassette (SEQ ID NO: 6) was replaced with variant OCT-1 transcription factor binding sequences of each of SEQ ID NO: 27-SEQ ID NO: 30 or a random sequence of SEQ ID NO: 45 or a duplicated random sequence of SEQ ID NO: 46. A construct encoding only a GFP cassette (“GFP Control”) was used as a negative control. Higher luciferase activity was indicative of increased engineered guide RNA expression.

FIG. 6 shows a bar plot of a luciferase assay to evaluate expression of an SNCA-targeting engineered guide RNA (SEQ ID NO: 1274) under control of a mouse U7 promoter with various zinc finger 143 motifs. The original zinc finger 143 motif (SEQ ID NO: 20) in the SNCA-targeting guide RNA expression cassette (SEQ ID NO: 6) was replaced with variant zinc finger 143 motifs of each of SEQ ID NO: 24-SEQ ID NO: 26 or a random sequence of SEQ ID NO: 43. A construct encoding only a GFP cassette (“GFP Control”) was used as a negative control. Higher luciferase activity was indicative of increased engineered guide RNA expression.

FIG. 7 shows a bar plot of a luciferase assay to evaluate expression of an SNCA-targeting engineered guide RNA (SEQ ID NO: 1274) under control of a mouse U7 promoter with various proximal sequence elements (PSEs). The original PSE (SEQ ID NO: 22) in the SNCA-targeting guide RNA expression cassette (SEQ ID NO: 6) was replaced with variant PSEs of each of SEQ ID NO: 31-SEQ ID NO: 37 or a random sequence of SEQ ID NO: 44. A construct encoding only a GFP cassette (“GFP Control”) was used as a negative control. Higher luciferase activity was indicative of increased engineered guide RNA expression.

FIG. 8 shows a bar plot of a luciferase assay to evaluate expression of an SNCA-targeting engineered guide RNA (SEQ ID NO: 1274) under control of a mouse U7 promoter with various transcriptional termination sequences. The original termination sequence (SEQ ID NO: 23) in the SNCA-targeting guide RNA expression cassette (SEQ ID NO: 6) was replaced with variant termination sequences of each of SEQ ID NO: 40-SEQ ID NO: 42 or a random sequence of SEQ ID NO: 47. A construct encoding only a GFP cassette (“GFP Control”) was used as a negative control. Higher luciferase activity was indicative of increased engineered guide RNA expression.

FIG. 9A shows a bar plot of a luciferase assay to evaluate expression of a PMP22-targeting engineered guide RNA (SEQ ID NO: 1273) under control of a mouse U7 promoter with various combinations of engineered sequence elements. SEQ ID NO: 2 contained a variant PSE of SEQ ID NO: 31 relative to SEQ ID NO: 1. SEQ ID NO: 3 contained a variant termination sequence of SEQ ID NO: 41 relative to SEQ ID NO: 1. SEQ ID NO: 4 contained a variant PSE of SEQ ID NO: 31 and a variant termination sequence of SEQ ID NO: 41 relative to SEQ ID NO: 1. SEQ ID NO: 5 contained a variant PSE of SEQ ID NO: 31, a variant termination sequence of SEQ ID NO: 41, and a variant OCT-1 transcription factor binding sequence of SEQ ID NO: 28 relative to SEQ ID NO: 1. Expression was quantified relative to a construct encoding only a GFP cassette (“GFP”). Higher luciferase activity was indicative of increased guide RNA expression.

FIG. 9B shows a bar plot of a luciferase assay to evaluate expression of an SNCA-targeting engineered guide RNA (SEQ ID NO: 1274) under control of a mouse U7 promoter with various combinations of engineered sequence elements. Expression of the SNCA-targeting guide RNA was also tested under control of a human U1 promoter (SEQ ID NO: 13) and a human U7 promoter (SEQ ID NO: 14). SEQ ID NO: 9 contained a variant PSE of SEQ ID NO: 31 relative to SEQ ID NO: 6. SEQ ID NO: 10 contained a variant termination sequence of SEQ ID NO: 41 relative to SEQ ID NO: 6. SEQ ID NO: 11 contained a variant PSE of SEQ ID NO: 31 and a variant termination sequence of SEQ ID NO: 41 relative to SEQ ID NO: 6. SEQ ID NO: 12 contained a variant PSE of SEQ ID NO: 31, a variant termination sequence of SEQ ID NO: 41, and a variant OCT-1 transcription factor binding sequence of SEQ ID NO: 28 relative to SEQ ID NO: 6. Expression was quantified relative to a construct encoding only a GFP cassette (“GFP”). Higher luciferase activity was indicative of increased guide RNA expression.

FIG. 10A shows a bar plot of a guide quantification assay to evaluate expression of a PMP22-targeting engineered guide RNA (SEQ ID NO: 1273) under control of a mouse U7 promoter with various combinations of engineered sequence elements. SEQ ID NO: 2 contained a variant PSE of SEQ ID NO: 31 relative to SEQ ID NO: 1. SEQ ID NO: 3 contained a variant termination sequence of SEQ ID NO: 41 relative to SEQ ID NO: 1. SEQ ID NO: 4 contained a variant PSE of SEQ ID NO: 31 and a variant termination sequence of SEQ ID NO: 41 relative to SEQ ID NO: 1. SEQ ID NO: 5 contained a variant PSE of SEQ ID NO: 31, a variant termination sequence of SEQ ID NO: 41, and a variant OCT-1 transcription factor binding sequence of SEQ ID NO: 28 relative to SEQ ID NO: 1. Expression was quantified relative to a construct encoding only a GFP cassette (“GFP”). Higher guide to GAPDH ratio was indicative of increased guide RNA expression.

FIG. 10B shows a bar plot of a guide quantification assay to evaluate expression of an SNCA-targeting engineered guide RNA (SEQ ID NO: 1274) under control of a mouse U7 promoter with various combinations of engineered sequence elements. Expression of the SNCA-targeting guide RNA was also tested under control of a human U1 promoter (SEQ ID NO: 13) and a human U7 promoter (SEQ ID NO: 14). SEQ ID NO: 9 contained a variant PSE of SEQ ID NO: 31 relative to SEQ ID NO: 6. SEQ ID NO: 10 contained a variant termination sequence of SEQ ID NO: 41 relative to SEQ ID NO: 6. SEQ ID NO: 11 contained a variant PSE of SEQ ID NO: 31 and a variant termination sequence of SEQ ID NO: 41 relative to SEQ ID NO: 6. SEQ ID NO: 12 contained a variant PSE of SEQ ID NO: 31, a variant termination sequence of SEQ ID NO: 41, and a variant OCT-1 transcription factor binding sequence of SEQ ID NO: 28 relative to SEQ ID NO: 6. Expression was quantified relative to a construct encoding only a GFP cassette (“GFP”). Higher guide to GAPDH ratio was indicative of increased guide RNA expression.

FIG. 11A shows a bar plot of Sanger editing of an ATG sequence to GTG to evaluate expression and editing activity of a PMP22-targeting engineered guide RNA (SEQ ID NO: 1273) under control of a mouse U7 promoter with various combinations of engineered sequence elements. SEQ ID NO: 2 contained a variant PSE of SEQ ID NO: 31 relative to SEQ ID NO: 1. SEQ ID NO: 3 contained a variant termination sequence of SEQ ID NO: 41 relative to SEQ ID NO: 1. SEQ ID NO: 4 contained a variant PSE of SEQ ID NO: 31 and a variant termination sequence of SEQ ID NO: 41 relative to SEQ ID NO: 1. SEQ ID NO: 5 contained a variant PSE of SEQ ID NO: 31, a variant termination sequence of SEQ ID NO: 41, and a variant OCT-1 transcription factor binding sequence of SEQ ID NO: 28 relative to SEQ ID NO: 1. A construct encoding only a GFP cassette (“GFP”) was used as a negative control. Higher editing percent was indicative of increased guide RNA expression.

FIG. 11B shows a bar plot of Sanger editing of an ATG sequence to GTG to evaluate expression and editing activity of an SNCA-targeting engineered guide RNA (SEQ ID NO: 1274) under control of a mouse U7 promoter with various combinations of engineered sequence elements. SEQ ID NO: 9 contained a variant PSE of SEQ ID NO: 31 relative to SEQ ID NO: 6. SEQ ID NO: 10 contained a variant termination sequence of SEQ ID NO: 41 relative to SEQ ID NO: 6. SEQ ID NO: 11 contained a variant PSE of SEQ ID NO: 31 and a variant termination sequence of SEQ ID NO: 41 relative to SEQ ID NO: 6. SEQ ID NO: 12 contained a variant PSE of SEQ ID NO: 31, a variant termination sequence of SEQ ID NO: 41, and a variant OCT-1 transcription factor binding sequence of SEQ ID NO: 28 relative to SEQ ID NO: 6. A construct encoding only a GFP cassette (“GFP”) was used as a negative control. Higher editing percent was indicative of increased guide RNA expression.

FIG. 12A shows a bar plot of Sanger editing of −3 position residue to evaluate expression and editing activity of a PMP22-targeting engineered guide RNA (SEQ ID NO: 1273) under control of a mouse U7 promoter with various combinations of engineered sequence elements. SEQ ID NO: 2 contained a variant PSE of SEQ ID NO: 31 relative to SEQ ID NO: 1. SEQ ID NO: 3 contained a variant termination sequence of SEQ ID NO: 41 relative to SEQ ID NO: 1. SEQ ID NO: 4 contained a variant PSE of SEQ ID NO: 31 and a variant termination sequence of SEQ ID NO: 41 relative to SEQ ID NO: 1. SEQ ID NO: 5 contained a variant PSE of SEQ ID NO: 31, a variant termination sequence of SEQ ID NO: 41, and a variant OCT-1 transcription factor binding sequence of SEQ ID NO: 28 relative to SEQ ID NO: 1. A construct encoding only a GFP cassette (“GFP”) was used as a negative control. Higher editing percent was indicative of increased guide RNA expression.

FIG. 12B shows a bar plot of Sanger editing of a −5 position residue to evaluate expression and editing activity of an SNCA-targeting engineered guide RNA (SEQ ID NO: 1274) under control of a mouse U7 promoter with various combinations of engineered sequence elements. SEQ ID NO: 9 contained a variant PSE of SEQ ID NO: 31 relative to SEQ ID NO: 6. SEQ ID NO: 10 contained a variant termination sequence of SEQ ID NO: 41 relative to SEQ ID NO: 6. SEQ ID NO: 11 contained a variant PSE of SEQ ID NO: 31 and a variant termination sequence of SEQ ID NO: 41 relative to SEQ ID NO: 6. SEQ ID NO: 12 contained a variant PSE of SEQ ID NO: 31, a variant termination sequence of SEQ ID NO: 41, and a variant OCT-1 transcription factor binding sequence of SEQ ID NO: 28 relative to SEQ ID NO: 6. A construct encoding only a GFP cassette (“GFP”) was used as a negative control. Higher editing percent was indicative of increased guide RNA expression.

FIG. 13A shows a scatter plot with a linear fit showing the correlation between the results of the guide quantification assay of FIG. 10B and the luciferase assay of FIG. 9B.

FIG. 13B shows a scatter plot with a linear fit showing the correlation between the results of the Sanger editing assay of FIG. 11B and the luciferase assay of FIG. 9B.

FIG. 13C shows a scatter plot with a linear fit showing the correlation between the results of the guide quantification assay of FIG. 10B and the Sanger editing assay of FIG. 11B.

FIG. 14A shows a scatter plot with a linear fit showing the correlation between the results of the guide quantification assay of FIG. 10A and the luciferase assay of FIG. 9A.

FIG. 14B shows a scatter plot with a linear fit showing the correlation between the results of the Sanger editing assay of FIG. 12A and the luciferase assay of FIG. 9A.

FIG. 14C shows a scatter plot with a linear fit showing the correlation between the results of the guide quantification assay of FIG. 10A and the Sanger editing assay of FIG. 11A.

FIG. 15 shows a sequence with a single copy of a promoter variant integrated into the genome of a HEK293T cell (left) and a comparison of copy integration of an engineered guide RNA targeting RAB7A (top of FIG. 15 (Cont.)), GAPDH (middle of FIG. 15 (Cont.)), and SNCA (bottom of FIG. 15 (Cont.)). FIG. 15 discloses SEQ ID NO: 1283 and SEQ ID NO: 1284, respectively, in order of appearance.

FIG. 16 shows a legend of various exemplary structural features present in guide-target RNA scaffolds formed upon hybridization of a latent guide RNA of the present disclosure to a target RNA. Example structural features shown include an 8/7 asymmetric loop (i., 8 nucleotides on the target RNA side and 7 nucleotides on the guide RNA side), a 2/2 symmetric bulge (ii., 2 nucleotides on the target RNA side and 2 nucleotides on the guide RNA side), a 1/1 mismatch (iii., 1 nucleotide on the target RNA side and 1 nucleotide on the guide RNA side), a 5/5 symmetric internal loop (iv., 5 nucleotides on the target RNA side and 5 nucleotides on the guide RNA side), a 24 bp region (v., 24 nucleotides on the target RNA side base paired to 24 nucleotides on the guide RNA side), and a 2/3 asymmetric bulge (vi., 2 nucleotides on the target RNA side and 3 nucleotides on the guide RNA side). FIG. 16 discloses SEQ ID NO: 1285 and SEQ ID NO: 1286, respectively, in order of appearance.

FIG. 17A shows bar charts quantifying expression of an SNCA-targeting guide RNA (SEQ ID NO: 1274, left) or a PMP22-targeting guide RNA (SEQ ID NO: 1273, right) in ARPE-19 cells. Expression of the SNCA-targeting guide RNA in ARPE-19 cells (left) was compared for an expression cassette under control of a wild type mouse U7 promoter (SEQ ID NO: 6) or an expression cassette under control of an engineered mouse U7 promoter (SEQ ID NO: 12). Expression of the PMP22-targeting guide RNA in ARPE-19 cells (right) was compared for an expression cassette under control of a wild type mouse U7 promoter (SEQ ID NO: 1) or an expression cassette under control of an engineered mouse U7 promoter (SEQ ID NO: 5). The engineered expression cassettes of SEQ ID NO: 12 and SEQ ID NO: 5 included an engineered promoter of SEQ ID NO: 17, comprising an OCT-1 transcription factor binding sequence of SEQ ID NO: 28 and a PSE of SEQ ID NO: 31, and an engineered termination sequence of SEQ ID NO: 60, comprising a termination sequence motif of SEQ ID NO: 41. Expression was quantified relative to a construct encoding only a GFP cassette (“GFP”). Higher guide to GAPDH ratio was indicative of increased guide RNA expression.

FIG. 17B shows a bar chart quantifying expression of a SERPINA1-targeting guide RNA (SEQ ID NO: 61) in HepG2 cells. Expression of the SERPINA1-targeting guide RNA in HepG2 cells was compared for an expression cassette under control of a wild type mouse U7 promoter (“mU7-WT”) or an expression cassette under control of an engineered mouse U7 promoter (SEQ ID NO: 59). The engineered expression cassette of SEQ ID NO: 59 included an engineered promoter of SEQ ID NO: 16, comprising a PSE of SEQ ID NO: 31, and an engineered termination sequence of SEQ ID NO: 60, comprising a termination sequence motif of SEQ ID NO: 41. Expression was quantified relative to a construct encoding only a GFP cassette (“GFP”). Higher guide to GAPDH ratio was indicative of increased guide RNA expression.

FIG. 18 shows exemplary novel promoters of the present disclosure tested on antisense oligonucleotides for clinically relevant Duchenne muscular dystrophy (DMD) exon skipping in differentiated muscle cells. Engineered guide RNA expressing constructs were randomly integrated into the genome and evaluated after 10 days of myocyte differentiation.

FIG. 19A shows exemplary combinations of promoters, promoter variants, 3′ box termination sequence, and truncated 3′box termination sequence of the present disclosure for driving guide RNA expression.

FIG. 19B shows exemplary combinations of promoters, promoter variants, 3′ box termination sequence, and truncated 3′box termination sequence of the present disclosure for driving guide RNA expression.

FIG. 20A shows a bar chart quantifying expression of PMP22-targeting guide RNAs with a luciferase reporter (Reporter 1) in HEK293 cells. Expression of the PMP22-targeting guide RNA in HEK293 cells by PMP22-targeting engineered guide RNA constructs with the engineered promoter elements included in SEQ ID NO: 2, SEQ ID NO: 3, and SEQ ID NO: 5 had increased fold expression relative to the control mU7 wildtype guide RNA construct (SEQ ID NO: 1).

FIG. 20B shows a bar chart quantifying expression of SNCA-targeting guide RNAs with a luciferase reporter (Reporter 2) in HEK293 cells. Expression of the Reporter 2 guide RNA in HEK293 cells by the SNCA-targeting engineered guide RNA constructs with the engineered promoter elements included in SEQ ID NO: 9, SEQ ID NO: 10, and SEQ ID NO: 12 had increased fold expression relative to the control mU7 wildtype guide RNA construct (SEQ ID NO: 6).

FIG. 21A shows a bar chart with the left panel quantifying expression of a PMP22-targeting guide RNA with a luciferase reporter (Reporter 1) in HEK293T cells. Expression of the Reporter 1 guide RNA in HEK293T cells by PMP22-targeting engineered guide RNA constructs with the engineered promoter elements included in SEQ ID NO: 5 had increased fold expression relative to the control mU7 wildtype guide RNA construct (SEQ ID NO: 1), as well as increased expression when compared to a control PMP22-targeting guide RNA under the control of a wildtype human U1 promoter (SEQ ID NO: 13). Negative control expression was also quantified by a construct encoding only a GFP cassette (“GFP ctrl”). The right panel of FIG. 21A shows a bar chart quantifying expression of a SNCA-targeting guide RNA with a luciferase reporter (Reporter 2) in HEK293T cells. Expression of the Reporter 2 guide RNA in HEK293T cells by the SNCA-targeting engineered guide RNA constructs with the engineered promoter elements included in SEQ ID NO: 12 had increased fold expression relative to the control mU7 wildtype guide RNA construct (SEQ ID NO: 6). Negative control expression was also quantified by a construct encoding only a GFP cassette (“GFP ctrl”).

FIG. 21B shows a bar chart with a left panel quantifying expression of a PMP22-targeting guide RNA with a luciferase reporter (Reporter 1) in HEK293T cells. Expression of the Reporter 1 guide RNA in HEK293T cells by the engineered PMP22-targeting guide RNA under the control of the engineered hU1 promoter (SEQ ID NO: 1241) had greater fold expression relative to a control PMP22-targeting guide RNA under the control of the wildtype human U1 promoter (SEQ ID NO: 13). Negative control expression was also quantified by a construct encoding only a GFP cassette (“GFP”). The right panel of FIG. 21B shows a bar chart quantifying expression of a SNCA-targeting guide RNA with a luciferase reporter (Reporter 2) in HEK293T cells. Expression of the Reporter 2 guide RNA in HEK293T cells by the engineered SNCA-targeting guide RNA under the control of the engineered hU1 promoter (SEQ ID NO: 1241) had greater fold expression relative to the control hU1 wildtype guide RNA construct (SEQ ID NO: 7). Negative control expression was also quantified by a construct encoding only a GFP cassette (“GFP”).

FIG. 22A shows a bar chart quantifying expression of a SNCA guide RNA for constructs comprising a promoter sequence comprising a full-length WT mU7 promoter sequence (SEQ ID NO: 15), a variant of the WT mU7 promoter sequence with a 100 base deletion between the DSE and PSE promoter elements (SEQ ID NO: 1248), an engineered mU7 promoter sequence (SEQ ID NO: 17), or a variant of the engineered mU7 promoter sequence with a 100 base deletion between the DSE and PSE promoter elements (SEQ ID NO: 1249). Guide RNA expression was quantified via ddPCR and normalized to a housekeeping gene (GAPDH). Higher guide RNA expression to GAPDH expression (gRNA/GAPDH) ratio was indicative of increased guide RNA expression.

FIG. 22B shows a bar chart quantifying expression of a PMP22 guide RNA for expression cassette constructs comprising a promoter sequence comprising a full-length WT mU7 promoter sequence (SEQ ID NO: 15), a variant of the WT mU7 promoter sequence with a 100 base deletion between the DSE and PSE promoter elements (SEQ ID NO: 1248), an engineered mU7 promoter sequence (SEQ ID NO: 17), or a variant of the engineered mU7 promoter sequence with a 100 base deletion between the DSE and PSE promoter elements (SEQ ID NO: 1249). Guide RNA expression was quantified via ddPCR and normalized to a housekeeping gene (GAPDH). Higher guide RNA expression to GAPDH expression (gRNA/GAPDH) ratio was indicative of increased guide RNA expression.

FIG. 23 shows a bar chart quantifying Rab7a editing in expression cassette constructs comprising a promoter sequence comprising a full-length WT mU7 promoter sequence (SEQ ID NO: 15), a variant of the WT mU7 promoter sequence with a 50 base deletion between the DSE and PSE promoter elements (SEQ ID NO: 1258), a variant of the WT mU7 promoter sequence with a 75 base deletion between the DSE and PSE promoter elements (SEQ ID NO: 1259), a variant of the WT mU7 promoter sequence with a 100 base deletion between the DSE and PSE promoter elements (SEQ ID NO: 1248), a variant of the WT mU7 promoter sequence with a 126 base deletion between the DSE and PSE promoter elements (SEQ ID NO: 1260), and a variant of the WT mU7 promoter sequence with a 135 base deletion between the DSE and PSE promoter elements (SEQ ID NO: 1261).

FIG. 24 shows a bar chart quantifying expression of GFP by expression constructs with Herpesvirus saimiri U-RNA elements (HSUR). The HSUR elements were extracted from NCBI NC_001350 and incorporated downstream of a gRNA cassette with a RNU5B1 promoter (SEQ ID NO: 1250) and a GFP gRNA which targets a GFP-G67R reporter wherein deamination of an AGA codon to GGA restores fluorescence in a correlative fashion. The expression constructs were introduced as single copy by BxbI integrase and enriched by puromycin for 14 days. The GFP expression was quantified by the geometric mean of fluorescence intensity (GFP gMFI) by flow cytometry and cells were gated for mCherry fluorescence upstream to enable graphing only of the cells which were positive for the cassette. The GFP expression was quantified for expression constructs comprising the termination sequences of SEQ ID NO: 1266-SEQ ID NO: 1272 and compared to the expression of GFP from an expression construct with a termination sequence of SEQ ID NO: 1254.

FIG. 25A shows a bar chart quantifying expression of a GFP guide RNA for expression cassette constructs comprising a promoter sequence of SEQ ID NO: 17 and a termination sequence of SEQ ID NO: 60 (SEQ ID NO: 17/SEQ ID NO: 60), a promoter sequence of SEQ ID NO: 15 and a termination sequence of SEQ ID NO: 1243 (SEQ ID NO: 15/SEQ ID NO: 1243), a promoter sequence of SEQ ID NO: 1250 and a termination sequence of SEQ ID NO: 1254 (SEQ ID NO: 1250/SEQ ID NO: 1254), a promoter sequence of SEQ ID NO: 1252 and a termination sequence of SEQ ID NO: 1256 (SEQ ID NO: 1252/SEQ ID NO: 1256), a promoter sequence of SEQ ID NO: 1251 and a termination sequence of SEQ ID NO: 1255 (SEQ ID NO: 1251/SEQ ID NO: 1255), or a promoter sequence of SEQ ID NO: 1253 and a termination sequence of SEQ ID NO: 1257 (SEQ ID NO: 1253/SEQ ID NO: 1257). Guide RNA expression was quantified via ddPCR and normalized to a housekeeping gene (GAPDH). Higher guide RNA expression to GAPDH expression (gRNA/GAPDH) ratio was indicative of increased guide RNA expression.

FIG. 25B shows a bar chart quantifying expression of a SNCA guide RNA for expression cassette constructs comprising a promoter sequence of SEQ ID NO: 17 and a termination sequence of SEQ ID NO: 60 (SEQ ID NO: 17/SEQ ID NO: 60), a promoter sequence of SEQ ID NO: 15 and a termination sequence of SEQ ID NO: 1243 (SEQ ID NO: 15/SEQ ID NO: 1243), a promoter sequence of SEQ ID NO: 1250 and a termination sequence of SEQ ID NO: 1254 (SEQ ID NO: 1250/SEQ ID NO: 1254), a promoter sequence of SEQ ID NO: 1252 and a termination sequence of SEQ ID NO: 1256 (SEQ ID NO: 1252/SEQ ID NO: 1256), a promoter sequence of SEQ ID NO: 1251 and a termination sequence of SEQ ID NO: 1255 (SEQ ID NO: 1251/SEQ ID NO: 1255), or a promoter sequence of SEQ ID NO: 1253 and a termination sequence of SEQ ID NO: 1257 (SEQ ID NO: 1253/SEQ ID NO: 1257). Guide RNA expression was quantified via ddPCR and normalized to a housekeeping gene (GAPDH). Higher guide RNA expression to GAPDH expression (gRNA/GAPDH) ratio was indicative of increased guide RNA expression.

FIG. 26 shows a schematic of a flow-seq pipeline for screening of promoter or termination sequences. The screen begins with a pool of HEK293 cells with a single attp1 sequence. The next intermediate generated contains two cassettes, one with the GFP-G67R ORF which has no fluorescence but a BFP for indication of enrichment. The second cassette contains Blasticidin resistance as well as the BxbI integrase. The library of promoters or termination sequences are cloned into a plasmid containing mCherry and puromycin resistance. The pooled promoter or termination sequence plasmid prep can be transfected into the intermediate cells and enriched for integrations by puromycin resistance with mCherry as a marker of enrichment.

FIG. 27 shows the results from the flowseq analysis described in FIG. 26, with the points representing the normalized performance of each termination sequence pooled from each of three promoter sequences. The arrow-indicated data points indicate superior termination sequences that were advanced into a single copy assessment including SEQ ID NO: 1254 and SEQ ID NO: 1255 that showed similar expression compared to a WT mU7 termination sequence (SEQ ID NO: 1243).

FIG. 28 shows a bar chart quantifying expression of GFP by expression constructs with the termination sequences identified in the flowseq screen, as described in FIG. 27. The GFP expression was quantified by the geometric mean of fluorescence intensity (GFP gMFI) by flow cytometry. The GFP expression was quantified for expression cassettes comprising termination sequences of SEQ ID NO: 712, SEQ ID NO: 868, SEQ ID NO: 1021, SEQ ID NO: 930, SEQ ID NO: 1017, SEQ ID NO: 1254, SEQ ID NO: 771, SEQ ID NO: 906, SEQ ID NO: 1007, and SEQ ID NO: 1002 and were compared to the engineered mU7 termination sequence of SEQ ID NO: 60.

### DETAILED DESCRIPTION

The present disclosure provides expression cassettes for expressing RNA payloads. The expression cassettes described herein may be engineered for increased expression of the encoded RNA payload sequence. In some embodiments, certain elements of the expression cassette, such as enhancer sequences, core promoter sequences, or transcriptional termination sequences, may be engineered for enhanced payload expression. These sequence elements may be engineered from various endogenous promoters, such as U1, U6, or U7 promoters, for increased payload expression. The individual sequence elements of the expression cassette may be engineered to enhance expression of the encoded RNA payload.

### Promoters and Termination Sequences

An expression cassette of the present disclosure may include a promoter sequence, an RNA payload coding sequence, and a termination sequence. The promoter may recruit transcription factors, polymerases (e.g., RNA polymerase II or RNA polymerase III), or other transcriptional machinery to promote transcription of the RNA payload. For example, the expression cassette may promote transcription of a guide RNA for RNA editing, a guide RNA for DNA editing, a tracrRNA, an siRNA, an shRNA, or a miRNA, or an antisense oligonucleotide). In some embodiments, the promoter may be engineered for increased expression of the RNA payload under transcriptional control of the promoter. The termination sequence may enhance termination of transcription and promote transcriptional turnover, increasing transcription of the payload. In some embodiments, the termination sequence may be engineered for enhanced expression of the RNA payload. Sequence elements within the promoter or termination sequence (e.g., transcription factor binding sequences, transcription initiation sequences, termination sequences, or combinations thereof) may be engineered for enhanced payload expression. The sequence elements may be interchangeable with sequence elements from endogenous RNA promoters, such as U1, U6, or U7 promoters.

An expression cassette may be engineered from an endogenous sequence. For example, an expression cassette may be engineered from an endogenous U1, U2, U3, U4, U5, U6, or U7 sequence. The endogenous sequence may be from any organism, including human, mouse, or other mammals. In some embodiments, an expression cassette may comprise a promoter engineered from an endogenous promoter, such as an endogenous U1, U2, U3, U4, U5, U6, or U7 promoter. In some embodiments, an expression cassette may comprise a transcriptional termination sequence engineered from an endogenous transcriptional termination sequence, such as an endogenous U1, U2, U3, U4, U5, U6, or U7 transcriptional termination sequence.

The present disclosure provides for regulatory elements that serve to enhance optimal expression of a small RNA payload, such as an engineered guide RNA. Regulatory elements can refer to a number of different regions in the native human genome, but as disclosed here, have been screened in large format assays to identify the combination of regulatory elements that provides for enhanced guide RNA expression. An expression cassette of the present disclosure includes both regulatory elements and payloads. For example, an expression cassette may include regulatory elements that comprise portions of native human genome or native mouse genome promoter regions. In some embodiments, the expression cassette may include regulatory elements that comprise Herpesvirus saimiri U-RNA (HSUR) elements. In some embodiments, the expression cassette may include regulatory elements that comprise mutated versions of native human genome promoter regions or mutated versions of native mouse genome promoter regions. In some embodiments, a vector of the present disclosure provides for two expression cassettes in which a native promoter region and a mutated promoter region are present. The expression cassettes of the present disclosure are engineered to position the promoter region 5′, or upstream, of a therapeutic payload (e.g., a small RNA sequence such as an engineered guide RNA).

Furthermore, regulatory elements can comprise portions of native human genome termination regions, native mouse genome termination sequences, or Herpesvirus saimiri U-RNA (HSUR) termination sequences. The regulatory elements can also comprise portions of mutated human genome termination regions or mutated mouse genome termination sequences. In some embodiments, a vector of the present disclosure provides for two expression cassettes in which a native termination region and a mutated termination region are present. The expression cassettes of the present disclosure are engineered to position the termination region 3′, or downstream, of the therapeutic payload.

The promoter regions of the present disclosure can be broken down into multiple elements, including (from 5′ to 3′) a distal sequence element (DSE) and a proximal sequence element (PSE). These different elements can play different roles in the rate and efficiency of transcription of the downstream payload. In some embodiments, the PSE is part of a core promoter region. The PSE may be bound by the snRNA activating protein complex (SNAPc). SNAPc is a transcription factor important for transcription initiation and may facilitate binding or recruitment of additional transcription factors (e.g., TBP, TFIIA, TFIIB, TFIIE and TFIIF). In some embodiments, the DSE is part of an enhancer region. The DSE may bind factors that help to stabilize transcription factors and transcription machinery on the PSE. In some embodiments, the DSE comprises an SPH element that recruits the STAF transcription factor (e.g., ZNF143 transcription factor). The STAF transcription factor (e.g., ZNF143 transcription factor) is a zinc finger protein and comprises activation domains that can active RNA polymerase promoters (e.g., mRNA-type RNA polymerase II promoters, type 3 RNA polymerase III promoters, and RNA polymerase II snRNA promoters). SPH elements may also comprise ZNF143 motifs capable of recruiting Zinc-finger 143 (ZNF143) transcription factors. In some embodiments, the DSE comprises an OCT-1 element that comprises an octamer sequence which recruits the Oct-1 transcription factor. Modifications to any one of the DSE and PSE regions, or other parts of the promoter region, or combinatorial selection of different DSE and PSE regions can improve the rate and efficiency of transcription of the downstream payload. The distance between the DSE and PSE can be varied. In some embodiments, the distance between the DSE and PSE is shortened compared to the native promoter sequence. In some embodiments, the distance between the DSE and PSE is extended compared to the native promoter sequence. In some embodiments, the present disclosure provides promoters from the native human genome that have been adapted for use in a heterologous system where transcription of a therapeutic payload is desired. In some embodiments, the present disclosure provides promoters that have modifications in the DSE as compared to a native human genome DSE or a native mouse genome DSE, which are part of the enhancer region of the promoter. Regions of the DSE that are important for engineering include the SPH element (recruiting the transcription factor STAF) and the OCT-1 transcription factor (TF) binding sequence. In some embodiments, the SPH element comprises a zinc finger 143 (ZNF143) motif (recruits zinc fingers). In some embodiments, the SPH element is a ZNF143 element (e.g., a zinc finger 143 (ZNF143) motif (recruits zinc fingers)). These SPH regions (e.g., ZNF143 motifs) and OCT-1 TF binding regions can also be referred to as regulatory factors. Promoter sequences, as disclosed herein, that have optimal elements within the DSE can result in enhanced transcription of the downstream small RNA payload. In some embodiments, promoter sequences of the present disclosure have one or more regions within them corresponding to an SPH element (e.g., a ZNF143 motif) and an OCT-1 TF binding sequence.

### Sequence Elements

Engineering an expression cassette may comprise incorporating or replacing an engineered sequence element into an expression construct. In some embodiments elements present in the DSE or PSE in the promoter may be incorporated or replaced with engineered elements. In some embodiments, sequence elements present in the termination sequence may be incorporated or replaced with engineered elements. For example, an endogenous transcription factor binding sequence present in the DSE (e.g., an endogenous SPH element such as a ZNF143-binding sequence, an endogenous OCT-1-binding sequence, or an endogenous GABP-binding sequence) may be replaced with an engineered transcription factor binding sequence (e.g., an engineered SPH element such as a ZNF143-binding sequence, an engineered OCT-1-binding sequence, or an engineered GABP-binding sequence). Alternatively, or in addition, an endogenous core promoter sequence element (e.g., an endogenous proximal sequence element or an endogenous TATA box) may be replaced by an engineered core promoter sequence (e.g., an engineered proximal sequence element or an engineered TATA box). Alternatively, or in addition, an endogenous termination sequence elements (e.g., an endogenous 3′ box sequence element) may be replaced by an engineered termination sequence element (e.g., an engineered 3′box sequence element). Examples of engineered sequence elements that may be inserted or substituted into an expression cassette are provided in TABLE 1.

TABLE 1
Exemplary Engineered Sequence Elements

| Sequence | | |
|---|---|---|
| Element | SEQ ID NO: | Sequence |
| ZNF143 | SEQ ID NO: 24 | ACTACAATTCCCAGC |
| ZNF143 | SEQ ID NO: 25 | TTCCCAGCATGCCCCGCGC |
| ZNF143 | SEQ ID NO: 26 | TACCCACAATGCCCTGC |
| OCT-1 | SEQ ID NO: 27 | ATGCAAAT |
| OCT-1 | SEQ ID NO: 28 | ATGCAAATCAAGAGAAATGCAAAT |
| OCT-1 | SEQ ID NO: 29 | ATGCATATTCAGCAAGAGAACTGC |
| | | ATATTCAT |
| OCT-1 | SEQ ID NO: 30 | ATTTGCATCAAGAGAAATTTGCAT |
| PSE | SEQ ID NO: 31 | AAGTCACCATGAGTGTAAAGGG |
| PSE | SEQ ID NO: 32 | AGGTCACCGTAACTATAAAAGA |
| PSE | SEQ ID NO: 33 | ACTTGACCTAAGTGTAAAGTT |
| PSE | SEQ ID NO: 34 | AAGTTACCATTACCCGTTTAGG |
| PSE | SEQ ID NO: 35 | AAATCACCATAAACGTGAAATG |
| PSE | SEQ ID NO: 36 | AAGTGACCTTGCGTGTAAAGGG |
| PSE | SEQ ID NO: 37 | AATGATCCTATATTTAGAGTGG |
| 3′ box | SEQ ID NO: 38 | GTTYN₀₋₃AARRYAGA |
| 3′ box | SEQ ID NO: 39 | GTTTN₁₋₄AANARNAGA |
| 3′ box | SEQ ID NO: 40 | GTTTAATAAAAATAGA |
| 3′ box | SEQ ID NO: 41 | GTTTCAAAAACAGA |
| 3′ box | SEQ ID NO: 42 | GTTCAATGGCTGA |

In some embodiments, an expression cassette may comprise one or more of the engineered sequence elements provided in TABLE 1. For example, an expression cassette may comprise a DSE with an engineered SPH element (e.g., a ZNF143 element) comprising a zinc finger 143 motif of any of SEQ ID NO: 24-SEQ ID NO: 26 that binds a ZNF143 transcription factor, a DSE with an engineered OCT-1 transcription factor binding site of any of SEQ ID NO: 27-SEQ ID NO: 30 that binds an OCT-1 transcription factor, an engineered proximal sequence element (PSE) of any of SEQ ID NO: 31-SEQ ID NO: 37 that recruits SNAPc and phosphorylated RNA polymerase II transcriptional machinery, an engineered transcriptional termination sequence element (e.g., a 3′ box sequence element) of any of SEQ ID NO: 38-SEQ ID NO: 42 that promotes termination of transcription, or combinations thereof.

An engineered SPH element comprising a zinc finger 143 motif may have at least about 70%, at least about 75%, at least about 80%, at least about 83%, at least about 85%, at least about 87%, at least about 90%, at least about 93%, at least about 95%, at least about 97%, or about 100% sequence identity to any of SEQ ID NO: 24-SEQ ID NO: 26. In some embodiments, the SPH element comprising a engineered zinc finger 143 motif may replace an endogenous SPH element comprising a zinc finger 143 motif of SEQ ID NO: 20.

An engineered OCT-1 transcription factor binding site may have at least about 7000, at least about 75%, at least about 80%, at least about 83%, at least about 85%, at least about 87%, at least about 90%, at least about 93%, at least about 95%, at least about 97%, or about 100% sequence identity to any of SEQ ID NO: 27-SEQ ID NO: 30. In some embodiments, an engineered OCT-1 transcription factor binding site may replace an endogenous OCT-1 transcription factor binding site of SEQ ID NO: 21 in the distal sequence element (DSE).

Additional exemplary PSE sequences of the present disclosure are provided in TABLE 2.

TABLE 2
Additional Exemplary PSE Sequences

| SEQ ID NO | Sequence |
|---|---|
| SEQ ID NO: 67 | AAATCACCATAAACGTGAAATG |
| SEQ ID NO: 68 | AAGTGACCGTGTGTGTAAAGAG |
| SEQ ID NO: 69 | AAGTGACCATGTGTGTAAAGGG |
| SEQ ID NO: 70 | AAGTGACCGTGCGTGTAAGGGG |
| SEQ ID NO: 71 | AAGTGACCGTGTGTGTAAAGGG |
| SEQ ID NO: 72 | TCAGCACCATTTTTTGGATCTG |
| SEQ ID NO: 73 | ATCTTACTTTTATGATCAATGT |
| SEQ ID NO: 74 | AAGTGACCGTGTGTTGAGAGTG |
| SEQ ID NO: 75 | GGCAAACTATGTTAAGGGAAGT |
| SEQ ID NO: 76 | AAGTGACCTTGCGTGTAAAGGG |
| SEQ ID NO: 77 | AAGTGACCGTGCGTGTAAAGGG |
| SEQ ID NO: 78 | AATTTGCCATGAGTATGTTGTG |
| SEQ ID NO: 79 | AATGATCCTATATTTAGAGTGG |
| SEQ ID NO: 80 | GAAACTCCATCTTAAAAAAAAA |
| SEQ ID NO: 81 | TAGTTACCATAACTGGTTGGAA |
| SEQ ID NO: 82 | TTCTTACCGTGACCTCAGGATG |
| SEQ ID NO: 83 | TTCTCGCCATCAGTTAAAAGTT |
| SEQ ID NO: 84 | TACTCACCATCAGCATAATATG |
| SEQ ID NO: 85 | CACTCACCCTCAATGTAATGGT |
| SEQ ID NO: 86 | AACTCACCTTTGCGAAATAGGA |
| SEQ ID NO: 87 | TAATTACCACAACCCTACCAGG |
| SEQ ID NO: 88 | TAGACACCATCAGTGTACTAGG |
| SEQ ID NO: 89 | TAGTAACCATTGCTAATCTAGT |
| SEQ ID NO: 90 | AAGTTACCATTACCCGTTTAGG |
| SEQ ID NO: 91 | AAGGCACCGTAAGTAGAGGGAG |
| SEQ ID NO: 92 | TAGGCACCATCGGCGTACTAGG |
| SEQ ID NO: 93 | TAGTCACCATCACTATACTAGG |
| SEQ ID NO: 94 | AATTTACCATTAGCCTGTTGGG |
| SEQ ID NO: 95 | CAACAACCATAAGTGTGTTAAG |
| SEQ ID NO: 96 | AGCTCACCCTCATCAATTGTGG |
| SEQ ID NO: 97 | AACTCACCCTAGCTTGTAACGG |
| SEQ ID NO: 98 | TGTTCACCTTTACCAAAAAATG |
| SEQ ID NO: 99 | TAGTCATCATACGCCTAATGAG |
| SEQ ID NO: 100 | TAGTCACCCTATGTGTAAATTA |
| SEQ ID NO: 101 | TACTCACCCTCAGCTGAAAATG |
| SEQ ID NO: 102 | AAGTTACCCCGATGACTTGGTT |
| SEQ ID NO: 103 | AACTCACCATAACTAAGAGAAG |
| SEQ ID NO: 104 | TATAAACCATGCCCAAAGGCTT |
| SEQ ID NO: 105 | CTGTCACCCTGAGGTTAGGATG |
| SEQ ID NO: 106 | TTTAAACCTGCTGTTTTGAAGA |
| SEQ ID NO: 107 | TTCTCACCCTAATCATAAAACA |
| SEQ ID NO: 108 | ACTTGACCTAAGTGTAAAGTT |
| SEQ ID NO: 109 | TGCTTACCGTAACTTGAAAGTA |
| SEQ ID NO: 110 | AGTTATCCTAACCAAAAGATG |
| SEQ ID NO: 111 | AGGTTACCGTAAGGAAAACAAA |
| SEQ ID NO: 112 | CGGTCACCGTAAGTAGAATAGG |
| SEQ ID NO: 113 | AGTCGGCCTATGTGTACAGAC |
| SEQ ID NO: 114 | AAGTCACCCTCACCGAAAGGCG |
| SEQ ID NO: 115 | ATATCACTGTAAGGGGAAAATG |
| SEQ ID NO: 116 | ACTCATCCTAACTTATTTAGA |
| SEQ ID NO: 117 | AAGTCTCCTTACCTAGAAAAGA |
| SEQ ID NO: 118 | ACGCGACCATAACTCTAAAAGG |
| SEQ ID NO: 119 | AAGTCACCATGAGTGTAAAGGG |
| SEQ ID NO: 120 | AGGTCACCGTAACTATAAAAGA |

A PSE may have at least about 70%, at least about 75%, at least about 80%, at least about 83%, at least about 85%, at least about 87%, at least about 90%, at least about 93%, at least about 95%, at least about 97%, or about 100% sequence identity to any of SEQ ID NO: 31-SEQ ID NO: 37 or SEQ ID NO: 67-SEQ ID NO: 120. In some embodiments, the PSE may replace an endogenous PSE of SEQ ID NO: 22. In some embodiments, a PSE that may be included in an engineered promoter sequence may have at least about 70%, at least about 75%, at least about 80%, at least about 83%, at least about 85%, at least about 87%, at least about 90%, at least about 93%, at least about 95%, at least about 97%, or about 100% sequence identity to any of SEQ ID NO: 67-SEQ ID NO: 120. In some embodiments, the promoter sequence comprises a PSE sequence of SEQ ID NO: 31-SEQ ID NO: 37 or SEQ ID NO: 67-SEQ ID NO: 120. In some embodiments, the PSE is selected from SEQ ID NO: 31-SEQ ID NO: 37 or SEQ ID NO: 67-SEQ ID NO: 120. The PSE may be selected or engineered from the PSE of an endogenous gene. For example, the PSE may have at least about 70%, at least about 75%, at least about 80%, at least about 83%, at least about 85%, at least about 87%, at least about 90%, at least about 93%, at least about 95%, at least about 97%, or about 100% sequence identity to a PSE from a U1, U2, U4, U5, U6, U7, U3, SNORD13, SNORD118, RPPH1, TRNAU1, 7SK, RNY3, or RNY4 gene. In some embodiments, an engineered promoter may include a PSE (e.g., any of SEQ ID NO: 31-SEQ ID NO: 37 or SEQ ID NO: 67-SEQ ID NO: 120). In some embodiments, an engineered promoter may include a PSE (e.g., any of SEQ ID NO: 31-SEQ ID NO: 37 or SEQ ID NO: 67-SEQ ID NO: 120) in place of a PSE of SEQ ID NO: 22.

In some embodiments, an engineered promoter may comprise a duplicated sequence element (e.g., a duplicated transcription factor binding site) to enhance payload expression. For example, an engineered promoter may comprise a DSE with two or more SPH elements comprising zinc finger 143 motifs (e.g., two or more of SEQ ID NO: 20 or SEQ ID NO: 24-SEQ ID NO: 26, or combinations thereof). In another example, an engineered promoter may comprise a DSE with two or more OCT-1 transcription factor binding sites (e.g., two or more of SEQ ID NO: 21 or SEQ ID NO: 27-SEQ ID NO: 30, or combinations thereof). In another example, an engineered promoter may comprise two or more proximal sequence elements (PSEs) (e.g., two or more of SEQ ID NO: 22, SEQ ID NO: 31-SEQ ID NO: 37, SEQ ID NO: 67-SEQ ID NO: 120, or combinations thereof). Duplicated sequences may be separated by a spacer sequence.

In some embodiments, an engineered promoter may comprise multiple promoter elements (e.g., a SPH element comprising a zinc finger 143 motif, an OCT-1 transcription factor binding site, or a proximal sequence element). In some embodiments, an engineered promoter may comprise one or more of an SPH element comprising a engineered zinc finger 143 motif of any of SEQ ID NO: 24-SEQ ID NO: 26 that binds a ZNF143 transcription factor, one or more of an engineered OCT-1 transcription factor binding site of any of SEQ ID NO: 27-SEQ ID NO: 30 that binds an OCT-1 transcription factor, or one or more of an engineered proximal sequence element (PSE) of any of SEQ ID NO: 31-SEQ ID NO: 37, SEQ ID NO: 67-SEQ ID NO: 120. An engineered promoter may also comprise an endogenous SPH element comprising a zinc finger 143 motif of SEQ ID NO: 20, an endogenous OCT-1 transcription factor binding site of SEQ ID NO: 21, or an endogenous proximal sequence element (PSE) of SEQ ID NO: 22.

An engineered transcriptional termination sequence may comprise a 3′ box sequence element. A 3′ box element may have at least about 70%, at least about 75%, at least about 80%, at least about 83%, at least about 85%, at least about 87%, at least about 90%, at least about 93%, at least about 95%, at least about 97%, or about 100% sequence identity to any of SEQ ID NO: 40-SEQ ID NO: 42. In some embodiments, a 3′ box sequence element may comprise a sequence of GTTYN₀₋₃AARRYAGA (SEQ ID NO: 38), wherein each N is independently A, T, C, or G, each R is independently A or G, and each Y is independently C or T. In some embodiments, a 3′ box element may comprise a sequence of GTTTN₁₋₄AANARNAGA (SEQ ID NO: 39), wherein each N is independently A, T, C, or G, and each R is independently A or G. In some embodiments, the engineered transcriptional termination sequence may replace an endogenous 3′ box sequence element of SEQ ID NO: 23.

Additional exemplary 3′ box sequence elements that may be included in an engineered termination sequence of the present disclosure are provided in TABLE 3.

TABLE 3
Additional Exemplary 3′ Box Sequence Elements

| SEQ ID NO | Sequence |
|---|---|
| SEQ ID NO: 121 | GTTCAATGGCTGA |
| SEQ ID NO: 122 | GTTTCAAAAACAGA |
| SEQ ID NO: 123 | GTTTCTAAAAGTAGA |
| SEQ ID NO: 124 | ATGAAAAAATAGA |
| SEQ ID NO: 125 | CTGGAAAACGCAGA |
| SEQ ID NO: 126 | GTTTAAAGAATAGT |
| SEQ ID NO: 127 | GTTTGATGTTAGA |
| SEQ ID NO: 128 | GTTGAAAGGTAGC |
| SEQ ID NO: 129 | GTGTAAAAAGCAGT |
| SEQ ID NO: 130 | GTTTTAAAAATAGG |
| SEQ ID NO: 131 | GTGGAAAGATAGA |
| SEQ ID NO: 132 | GTGCGAATAGTAGG |
| SEQ ID NO: 133 | GTTTTAAAAGTGGA |
| SEQ ID NO: 134 | GTTTAAAAGACGG |
| SEQ ID NO: 135 | GTTTATAAAAGGC |
| SEQ ID NO: 136 | ATTAAAAGAAATA |
| SEQ ID NO: 137 | GTTTAATGGAAGA |
| SEQ ID NO: 138 | TTTACAAAGAACAGA |
| SEQ ID NO: 139 | GCTCAATGACAGA |
| SEQ ID NO: 140 | TCTAGAGAAGGCAGT |
| SEQ ID NO: 141 | ATGTTAATAGTAGT |
| SEQ ID NO: 142 | GTCTAAAGAAAAGG |
| SEQ ID NO: 143 | GTTGAACAACAGA |
| SEQ ID NO: 144 | GTTCAAACAGCAGT |
| SEQ ID NO: 145 | ATCCAACAATAGA |
| SEQ ID NO: 146 | GTTTAAAAATCAGA |
| SEQ ID NO: 147 | GTTTTAAAAACAGA |
| SEQ ID NO: 148 | GTTACTAAAGAGAGA |
| SEQ ID NO: 149 | GTTTTATAAAAAAAGA |
| SEQ ID NO: 150 | GTTAAAAAATCAGA |
| SEQ ID NO: 151 | GTTTTAAAAGTAGA |
| SEQ ID NO: 152 | ATTAAAAGTTAGG |
| SEQ ID NO: 153 | GTTGCCAATGATAGA |
| SEQ ID NO: 154 | GCTGATTAGCAGA |
| SEQ ID NO: 155 | GTTAGGCGAAATATT |
| SEQ ID NO: 156 | GTAACTGAAAAGAGA |
| SEQ ID NO: 157 | GCCTAAAAAGTAGA |
| SEQ ID NO: 158 | CTTCAAGGATCGA |
| SEQ ID NO: 159 | GCTGCAAGGTCAGG |
| SEQ ID NO: 160 | GTTCAAGAGCAGT |
| SEQ ID NO: 161 | AAGAAAAAGAAGA |
| SEQ ID NO: 162 | GACCAAAGGCAGG |
| SEQ ID NO: 163 | CTTCAAACAAAGG |
| SEQ ID NO: 164 | GTCGCTAACGGGAGA |
| SEQ ID NO: 165 | TTTTTAGATTAATAGA |
| SEQ ID NO: 166 | GTTTAATAAAAATAGA |

An engineered 3′ box sequence element may have at least about 7000, at least about 75%, at least about 80%, at least about 83%, at least about 85%, at least about 87%, at least about 90%, at least about 93%, at least about 95%, at least about 97%, or about 100% sequence identity to any of SEQ ID NO: 40-SEQ ID NO: 42 or SEQ ID NO: 121-SEQ ID NO: 166. In some embodiments, the engineered transcriptional termination sequence may replace an endogenous 3′ box sequence element of SEQ ID NO: 23. In some embodiments, a 3′ box sequence element that may be included in an engineered promoter sequence may have at least about 70%, at least about 75%, at least about 80%, at least about 83%, at least about 85%, at least about 87%, at least about 90%, at least about 93%, at least about 95%, at least about 97%, or about 100% sequence identity to any of SEQ ID NO: 121-SEQ ID NO: 166. In some embodiments, the termination sequence comprises a 3′ box sequence element sequence of SEQ ID NO: 40-SEQ ID NO: 42 or SEQ ID NO: 121-SEQ ID NO: 166. In some embodiments, the 3′ box sequence element is selected from SEQ ID NO: 40-SEQ ID NO: 42 or SEQ ID NO: 121-SEQ ID NO: 166. The 3′ box sequence element may be selected or engineered from the 3′ box sequence element of an endogenous gene. For example, the 3′ box sequence element may have at least about 70%, at least about 75%, at least about 80%, at least about 83%, at least about 85%, at least about 87%, at least about 90%, at least about 93%, at least about 95%, at least about 97%, or about 100% sequence identity to a 3′ box sequence element from a U1, U2, U4, U5, U6, U7, U3, SNORD13, SNORD118, RPPH1, TRNAU1, 7SK, RNY3, or RNY4 gene. In some embodiments, an engineered termination sequence may include a 3′ box sequence element (e.g., any of SEQ ID NO: 40-SEQ ID NO: 42 or SEQ ID NO: 121-SEQ ID NO: 166). In some embodiments, an engineered termination sequence may include a 3′ box sequence element (e.g., any of SEQ ID NO: 40-SEQ ID NO: 42 or SEQ ID NO: 121-SEQ ID NO: 166) in place of a 3′ box sequence element of SEQ ID NO: 23.

### Promoters

An expression cassette may comprise a promoter. A promoter may be an endogenous promoter. A promoter may be an engineered promoter engineered to increase expression of an RNA payload sequence under transcriptional control of the promoter. Examples of endogenous promoters (e.g., SEQ ID NO: 13-SEQ ID NO: 15), engineered promoters (e.g., SEQ ID NO: 16, SEQ ID NO: 17, SEQ ID NO: 1241, SEQ ID NO: 1248, SEQ ID NO: 1249, SEQ ID NO: 1252, SEQ ID NO: 1253, and SEQ ID NO: 1258-SEQ ID NO: 1261), and additional promoters (e.g., SEQ ID NO: 1250, SEQ ID NO: 1251, SEQ ID NO: 1262, and SEQ ID NO: 1263) are provided in TABLE 4.

TABLE 4
Exemplary Promoter Sequences

| SEQ ID NO: | Sequence |
|---|---|
| SEQ ID NO: 13 | TAAGGACCAGCTTCTTTGGGAGAGAACAGACGCAGGGGGGGGAGGG |
| | AAAAAGGGAGAGGCAGACGTCACTTCCTCTTGGCGACTCTGGCAGCA |
| | GATTGGTCGGTTGAGTGGCAGAAAGGCAGACGGGGACTGGGCAAGG |
| | CACTGTCGGTGACATCACGGACAGGGCGACTTCTATGTAGATGAGGC |
| | AGCGCAGAGGCTGCTGCTTCGCCACTTGCTGCTTCGCCACGAAGGGA |
| | GTTCCCGTGCCCTGGGAGCGGGTTCAGGACCGCTGATCGGAAGTGAG |
| | AATCCCAGCTGTGTGTCAGGGCTGGAAAGGGCTCGGGAGTGCGCGGG |
| | GCAAGTGACCGTGTGTGTAAAGAGTGAGGCGTATGAGGCTGTGTCGG |
| | GGCAGAGCCCGAAGATCTC |
| SEQ ID NO: 14 | TTAACAACAACGAAGGGGCTGTGACTGGCTGCTTTCTCAACCAATCA |
| | GCACCGAACTCATTTGCATGGGCTGAGAACAAATGTTCGCGAACTCT |
| | AGAAATGAATGACTTAAGTAAGTTCCTTAGAATATTATTTTTCCTACT |
| | GAAAGTTACCACATGCGTCGTTGTTTATACAGTAATAGGAACAAGAA |
| | AAAAGTCACCTAAGCTCACCCTCATCAATTGTGGAGTTCCTTTATATC |
| | CCATCTTCTCTCCAAACACATACGCA |
| SEQ ID NO: 15 | TAACAACATAGGAGCTGTGATTGGCTGTTTTCAGCCAATCAGCACTGA |
| | CTCATTTGCATAGCCTTTACAAGCGGTCACAAACTCAAGAAACGAGC |
| | GGTTTTAATAGTCTTTTAGAATATTGTTTATCGAACCGAATAAGGAAC |
| | TGTGCTTTGTGATTCACATATCAGTGGAGGGGTGTGGAAATGGCACCT |
| | TGATCTCACCCTCATCGAAAGTGGAGTTGATGTCCTTCCCTGGCTCGC |
| | TACAGACGCACTTCCGC |
| SEQ ID NO: 16 | TAACAACATAGGAGCTGTGATTGGCTGTTTTCAGCCAATCAGCACTGA |
| | CTCATTTGCATAGCCTTTACAAGCGGTCACAAACTCAAGAAACGAGC |
| | GGTTTTAATAGTCTTTTAGAATATTGTTTATCGAACCGAATAAGGAAC |
| | TGTGCTTTGTGATTCACATATCAGTGGAGGGGTGTGGAAATGGCACCT |
| | TGATAAGTCACCATGAGTGTAAAGGGAGTTGATGTCCTTCCCTGGCTC |
| | GCTACAGACGCACTTCCGC |
| SEQ ID NO: 17 | TAACAACATAGGAGCTGTGATTGGCTGTTTTCAGCCAATCAGCACTGA |
| | CTCATGCAAATCAAGAGAAATGCAAATAGCCTTTACAAGCGGTCACA |
| | AACTCAAGAAACGAGCGGTTTTAATAGTCTTTTAGAATATTGTTTATC |
| | GAACCGAATAAGGAACTGTGCTTTGTGATTCACATATCAGTGGAGGG |
| | GTGTGGAAATGGCACCTTGATAAGTCACCATGAGTGTAAAGGGAGTT |
| | GATGTCCTTCCCTGGCTCGCTACAGACGCACTTCCGC |
| SEQ ID NO: | TAAGGACCAGCTTCTTTGGGAGAGAACAGACGCAGGGGGGGGAGGG |
| 1241 | AAAAAGGGAGAGGCAGACGTCACTTCCTCTTGGCGACTCTGGCAGCA |
| | GATTGGTCGGTTGAGTGGCAGAAAGGCAGACGGGGACTGGGCAAGG |
| | CACTGTCGGTGACATCACGGACAGGGCGACTTCTATGCAAATCAAGA |
| | GAAATGCAAATGAGGCAGCGCAGAGGCTGCTGCTTCGCCACTTGCTG |
| | CTTCGCCACGAAGGGAGTTCCCGTGCCCTGGGAGCGGGTTCAGGACC |
| | GCTGATCGGAAGTGAGAATCCCAGCTGTGTGTCAGGGCTGGAAAGGG |
| | CTCGGGAGTGCGCGGGGCAAAAGTCACCATGAGTGTAAAGGGTGAGG |
| | CGTATGAGGCTGTGTCGGGGCAGAGCCCGAAGATCTC |
| SEQ ID NO: | TAACAACATAGGAGCTGTGATTGGCTGTTTTCAGCCAATCAGCACTGA |
| 1248 | CTCATTTGCATAGCCTTTACAAGCGGTCATGGAAATGGCACCTTGATC |
| | TCACCCTCATCGAAAGTGGAGTTGATGTCCTTCCCTGGCTCGCTACAG |
| | ACGCACTTCCGC |
| SEQ ID NO: | TAACAACATAGGAGCTGTGATTGGCTGTTTTCAGCCAATCAGCACTGA |
| 1249 | CTCATGCAAATCAAGAGAAATGCAAATAGCCTTTACAAGCGGTCATG |
| | GAAATGGCACCTTGATAAGTCACCATGAGTGTAAAGGGAGTTGATGT |
| | CCTTCCCTGGCTCGCTACAGACGCACTTCCGC |
| SEQ ID NO: | TCGCCCCCTAACGGTGACATAAGGCACTCTGTGAAATGCTCTGTTCCG |
| 1250 | GAATCAAAAGATTGATCCGATTATTTGCATACCCATAATGCACTGCTC |
| | ACAGTACAAATTTAAAAAGGCAAAATCAAACATTTTTATTCTAAGCAT |
| | ATTCTGTGAAAGTTAGACTTTTGTTTAAACAATACTCTTAAAATTTTTT |
| | TCTAGGTATAGAACCTTGGCATTCACTAGTCACCATCACTATACTAGG |
| | AGTTTCTGTTACCCGAGAAACGAGTTATGAAATTAACAAGC |
| SEQ ID NO: | GTGGCCTCAGGCGCAGCGCTAAAACGCATGAACCATTTAAGGTATTT |
| 1251 | CCTGAAACTGGAGCGTGATTGGTGAGACTTTATTTGCATACCCACAAT |
| | GCATTGCGCACTAAATAATGTTCGTCTTTAAAATTATTTCCCCTTTTTC |
| | CTTCAACATATCTTTCTCGGAACCGAGATTGCTGTCCCAGAATTGTCT |
| | GAAGAAAAAGGCTGAAGTCAATAGCTCTTTTGGGCCGAAGGAAAGTT |
| | ACCATTACCCGTTTAGGAGTAGCCGTTACCTGAGAACTGTAGTGTCGA |
| | CGACTGATGTTAT |
| SEQ ID NO: | GTGGCCTCAGGCGCAGCGCTAAAACGCATGAACCATTTAAGGTATTT |
| 1252 | CCTGAAACTGGAGCGTGATTGGTGAGACTTTATGCAAATCAAGAGAA |
| | ATGCAAATACCCACAATGCATTGCGCACTAAATAATGTTCGTCTTTAA |
| | AATTATTTCCCCTTTTTCCTTCAACATATCTTTCTCGGAACCGAGATTG |
| | CTGTCCCAGAATTGTCTGAAGAAAAAGGCTGAAGTCAATAGCTCTTTT |
| | GGGCCGAAGGAAAGTCACCATGAGTGTAAAGGGAGTAGCCGTTACCT |
| | GAGAACTGTAGTGTCGACGACTGATGTTAT |
| SEQ ID NO: | GTCAAGTGCCTCTCTCCATTTACTGGTAAGAGAGAGAGGGTTTAGAG |
| 1253 | GAACTCTTGTTCCGGCGCTCAGCTCATGCAAATCAAGAGAAATGCAA |
| | ATCCCAGAATGCATTGTAGATACGAGAATTATTACCAGGGTTATCTGT |
| | TTGAATAATAATATTTAAACTTTTTTTCTTTGTCAGGAGATTTTACCCA |
| | GTGAGAACATGTTTAGGACACTTTTCTACAGTGGAAGAAAAGCTTCTG |
| | TCTGCAGGTCCATTCTCGCCATCAGTTAAAAGTTACCAGTCAATAGCT |
| | GGGAAGCCAGGCAAAAGGCTAACAGGCAG |
| SEQ ID NO: | TAACAACATAGGAGCTGTGATTGGCTGTTTTCAGCCAATCAGCACTGA |
| 1258 | CTCATTTGCATAGCCTTTACAAGCGGTCACAAACTCAAGAAACGAGC |
| | GGTTTTATGATTCACATATCAGTGGAGGGGTGTGGAAATGGCACCTTG |
| | ATCTCACCCTCATCGAAAGTGGAGTTGATGTCCTTCCCTGGCTCGCTA |
| | CAGACGCACTTCCGC |
| SEQ ID NO: | TAACAACATAGGAGCTGTGATTGGCTGTTTTCAGCCAATCAGCACTGA |
| 1259 | CTCATTTGCATAGCCTTTACAAGCGGTCACAAACTCAAGAAAAGTGG |
| | AGGGGTGTGGAAATGGCACCTTGATCTCACCCTCATCGAAAGTGGAG |
| | TTGATGTCCTTCCCTGGCTCGCTACAGACGCACTTCCGC |
| SEQ ID NO: | TAACAACATAGGAGCTGTGATTGGCTGTTTTCAGCCAATCAGCACTGA |
| 1260 | CTCATTTGCATAGCCTTTGATCTCACCCTCATCGAAAGTGGAGTTGAT |
| | GTCCTTCCCTGGCTCGCTACAGACGCACTTCCGC |
| SEQ ID NO: | TAACAACATAGGAGCTGTGATTGGCTGTTTTCAGCCAATCAGCACTGA |
| 1261 | CTCATTTGCATACTCACCCTCATCGAAAGTGGAGTTGATGTCCTTCCC |
| | TGGCTCGCTACAGACGCACTTCCGC |
| SEQ ID NO: | ATTTAATAGCAGTCTTTATTTAAAAGAAATCAAACTCAGACGTACAAA |
| 1262 | TACACAAAACAGATAAAACCCGAGTCTCTGACCAGGAAAGCGTTATT |
| | TTCCAGCCAGCCAGTCTTCGGCTTCGCCCCCTAACGGTGACATAAGGC |
| | ACTCTGTGAAATGCTCTGTTCCGGAATCAAAAGATTGATCCGATTATT |
| | TGCATACCCATAATGCACTGCTCACAGTACAAATTTAAAAAGGCAAA |
| | ATCAAACATTTTTATTCTAAGCATATTCTGTGAAAGTTAGACTTTTGTT |
| | TAAACAATACTCTTAAAATTTTTTTCTAGGTATAGAACCTTGGCATTC |
| | ACTAGTCACCATCACTATACTAGGAGTTTCTGTTACCCGAGAAACGAG |
| | TTATGAAATTAACAAGC |
| SEQ ID NO: | CAATTACTTTTGCACCGATCTAATAGCTCGCCCACTGTAAGTAAAGCA |
| 1263 | GGTAAAGTCAGCCTTTTTCTTCTGGGACCAGACTCTGCTCTGCCCCGC |
| | GGTGGTGGCCTCAGGCGCAGCGCTAAAACGCATGAACCATTTAAGGT |
| | ATTTCCTGAAACTGGAGCGTGATTGGTGAGACTTTATTTGCATACCCA |
| | CAATGCATTGCGCACTAAATAATGTTCGTCTTTAAAATTATTTCCCCTT |
| | TTTCCTTCAACATATCTTTCTCGGAACCGAGATTGCTGTCCCAGAATT |
| | GTCTGAAGAAAAAGGCTGAAGTCAATAGCTCTTTTGGGCCGAAGGAA |
| | AGTTACCATTACCCGTTTAGGAGTAGCCGTTACCTGAGAACTGTAGTG |
| | TCGACGACTGATGTTAT |

In some embodiments, a promoter for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to any of SEQ ID NO: 13-SEQ ID NO: 17, SEQ ID NO: 1241, SEQ ID NO: 1248-SEQ ID NO: 1253, or SEQ ID NO: 1259-SEQ ID NO: 1263. In some embodiments, an engineered promoter for enhanced expression of an RNA payload may be a variant of a promoter (e.g., a variant of any one of SEQ ID NO: 13-SEQ ID NO: 15, SEQ ID NO: 1250, SEQ ID NO: 1251, SEQ ID NO: 1262, and SEQ ID NO: 1263). In some embodiments, an engineered promoter may comprise a variant of any of SEQ ID NO: 13-SEQ ID NO: 15, SEQ ID NO: 1250, SEQ ID NO: 1251, SEQ ID NO: 1262, and SEQ ID NO: 1263 having at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to any of SEQ ID NO: 13-SEQ ID NO: 15, SEQ ID NO: 1250, SEQ ID NO: 1251, SEQ ID NO: 1262, and SEQ ID NO: 1263 and at least one nucleotide substitution relative to any of SEQ ID NO: 13-SEQ ID NO: 15, SEQ ID NO: 1250, SEQ ID NO: 1251, SEQ ID NO: 1262, and SEQ ID NO: 1263.

In some embodiments, a promoter for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 13. In some embodiments, a promoter for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 15. In some embodiments, a promoter for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 17. In some embodiments, a promoter for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1241. In some embodiments, a promoter for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1250. In some embodiments, a promoter for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1251. In some embodiments, a promoter for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1252. In some embodiments, a promoter for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1253. In some embodiments, a promoter for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1262. In some embodiments, a promoter for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1263.

An engineered promoter may enhance expression of an RNA payload under control of the engineered promoter relative to an endogenous promoter (e.g., an endogenous U1 promoter, an endogenous U6 promoter, or an endogenous U7 promoter). In some embodiments, the engineered promoter (e.g., a promoter comprising a sequence of any one of SEQ ID NO: 16, SEQ ID NO: 17, SEQ ID NO: 1241, SEQ ID NO: 1248, SEQ ID NO: 1249, SEQ ID NO: 1252, SEQ ID NO: 1253, or SEQ ID NO: 1258-SEQ ID NO: 1261) may increase expression of an RNA payload by at least about 5%, at least about 10%, at least about 15%, at least about 20%, at least about 25%, at least about 30%, at least about 35%, at least about 40%, at least about 45%, or at least about 50% relative to an endogenous promoter (e.g., an endogenous U1 promoter, an endogenous U6 promoter, or an endogenous U7 promoter). In some embodiments, the engineered promoter (e.g., a promoter comprising a sequence of any one of SEQ ID NO: 16, SEQ ID NO: 17, SEQ ID NO: 1241, SEQ ID NO: 1248, SEQ ID NO: 1249, SEQ ID NO: 1252, SEQ ID NO: 1253, and SEQ ID NO: 1258-SEQ ID NO: 1261) may increase expression of an RNA payload by from about 5% to about 50%, from about 10% to about 50%, from about 15% to about 50%, from about 20% to about 50%, from about 25% to about 50%, from about 30% to about 50%, from about 35% to about 50%, from about 40% to about 50%, from about 45% to about 50%, from about 5% to about 40%, from about 10% to about 40%, from about 15% to about 40%, from about 20% to about 40%, from about 25% to about 40%, from about 30% to about 40%, from about 35% to about 40%, from about 5% to about 30%, from about 10% to about 30%, from about 15% to about 30%, from about 20% to about 30%, from about 5% to about 30%, from about 10% to about 20%, or from about 15% to about 20% relative to an endogenous promoter (e.g., an endogenous U1 promoter, an endogenous U6 promoter, or an endogenous U7 promoter).

In some embodiments, a promoter sequence may enhance transcription of an RNA payload. The promoter sequence may be positioned upstream of the payload sequence. Additional exemplary promoter sequences of the present disclosure are provided in TABLE 5.

TABLE 5
Additional Exemplary Promoter Sequences

| SEQ ID NO | Sequence |
|---|---|
| SEQ ID NO: 167 | AATTGTACCATAAAAGAATCTTGAGGATATCTTTAAAAGGTCTGCTCTCTTACGAAGGTGAA |
| | GTGTCTCCCCTGTAAGCTTGTTTACACCGGCATCTGTTCAGCCAGCCCTTTTTCATAGACACT |
| | TCAGCCAAATACTGGCGTGTTTTCCGTTTCTGTGTTTACGGTTGGACTCAACGAGCCTTTTAC |
| | TATTGTGGCATATGGGAGGGAGCGTTGCCATTCTGCTGCCAGGGGTGAGTGTGATCTGGTG |
| | GCTGTTACATTGTGTATATGCGAGTAGGTCTCTATCAAGAAGGGACCCGCC |
| SEQ ID NO: 168 | TGGTGGCCTCAGGCGCAGCGCTAAAACGCATGAACCATTTAAGGTATTTCCTGAAACTGGA |
| | GCGTGATTGGTGAGACTTTATTTGCATACCCACAATGCATTGCGCACTAAATAATGTTCGTC |
| | TTTAAAATTATTTCCCCTTTTTCCTTCAACATATCTTTCTCGGAACCGAGATTGCTGTCCCAG |
| | AATTGTCTGAAGAAAAAGGCTGAAGTCAATAGCTCTTTTGGGCCGAAGGAAAGTTACCATT |
| | ACCCGTTTAGGAGTAGCCGTTACCTGAGAACTGTAGTGTCGACGACTGATGTT |
| SEQ ID NO: 169 | AGATTGGTCGGTTGAGTGGCAGAAAGGCAGACGGGGACTGGGCAAGGCACTGTCGGTGAC |
| | ATCACGGACAGGGCGACTTCTATGTAGATGAGGCAGCGCAGAGGCTGCTGCTTCGCCACTT |
| | GCTGCTTCGCCACGAAGGAGTTCCCGTGCCCTGGGAGCGGGTTCAGGACCGCGGATCGGAA |
| | GTGAGAATCCCAGCTGTGTGTCAGGGCTGGAAAGGGCTCGGGAGTGCGCGGGGCAAGTGA |
| | CCGTGTGTGTAAAGAGTGAGGCGTATGAGGCTGTGTCGGGGCAGAGCCCGAAGATCTC |
| SEQ ID NO: 170 | TCGGAAGAACCCCGAGTCCATTGTAAGCTCAGGGGAGAGCGGGAGCCAGGGAGGTGAAGT |
| | GCGCAGACTCGGCAGCGGCGGCGGGCAGAACCGCGGGGGGGTGAGAGGGCGCGGTGGCTG |
| | CGGGGCGGGAGCCGCTGCTGAGAGGCGGCCTGGGTTGTCTTGTGGGGTGACTGTCGGTGGA |
| | ATCTTTGGTGGAGAGTGGTTTGGAAGAATGGCGAGGGGCGGCAGTGGGGAGGGTGGTGAC |
| | CCTGAGCGACCGGCCAGGGCGAGGAGGCTGTGCTGTCCCTGCAGGCCATGTGCTCATTT |
| SEQ ID NO: 171 | AGATTGGTCAGTTGAGTGGCAGAAAAGCAGACGGGGACTGGGCAAGGCACTGTCGGTGAC |
| | ATCACGGACAGGGCGACTTCTATGTAGATGAGGCAGCGCAGAGGCTGCTGCTTCGCCACTT |
| | GCTGCTTCGCCACGAAGGAGTTCCCCTGCCCTGGGAGCGGGTTCAGGACCGCGGATCGGAA |
| | GAGAGAATCCCAGCTGTGTGTCAGGGCTGGAAAGGGCTCGGGAGTGCGCGGGGCAAGTGA |
| | CCGTGTGTGTAAAGAGTGAGGCGTATGAGGCTGTGTCGGGGCAGAGCCCGAAGATCTC |
| SEQ ID NO: 172 | AGCACTTTGAGACGCTGAGGTGGGTGGATCACCTGAGGTCAAGAGTTCAAGACCAGCCTGG |
| | CCAACATGGTGAAGCCTCATCTCTACCAAAAATACAAAAAAATTAGCCAGGCATGGTAGTG |
| | GGCACCTGTAATCCCAGCTATTCGGGAGGCTGAAGCAGGAGAATTGCTTGAACCCAGGAGG |
| | TGGAGGTTGCAGTGAGCCAAGATCGCACCACTGCACTCCAGCCTGGGCGACAGAGCAAGAC |
| | TCTGTCTCAAAACAAAACAAAATAAAACAAAACAAAAACAAACAGAAAAGCTAAGA |
| SEQ ID NO: 173 | TCCTTTCCAGGGTGGACTCCACTCCCACTCTCACAAAAACATGTAGTAGGCCCTGGGCATTC |
| | AGCTCTTCCACACACTGCATGGCTTCCTGTTCCAACAGCAAAGAAAGGTTTACTCCAAAATC |
| | TGAATAGTGTTACCAGAATGTTAAGATAACTTTAAAATCTTTTACTTACAACAGAATCAGTA |
| | GAAACTTCCATGAACAGGACAGTTTTCAAATATAAGTTTATATTTGTTTAACATAATTTATA |
| | GCTCAGTAAATTACAGGCTGATATGGGCAATATTAAAAGTCATACAAAAAAA |
| SEQ ID NO: 174 | GTTCCTCCCTCGCTCGCGCAGCCGCTCTTCCCCGCCACTCCCTCGGTGCCCGCCAGCACATTC |
| | CCAGCAAGCCCTGAGTATATTTGCATATCAACTCACTACATTTTTTTCTTCTAACTAAAAAAT |
| | CGAAAGGACAAATTCCAGATTCTCCTTGTGAAGTCTTCCTTTCAGTTCAGAAGAAATGGAAT |
| | TCGCTCTTCAACTTCAGGAAGTTGAAATAAAGAGTTGCTTGGATTTTGTGTTCACCTTTACC |
| | AAAAAATGGATTTGGTAACACTGCCACCCTGCTTTGGTGACAGAGAAAGC |
| SEQ ID NO: 175 | AAGATCATAGGATAGGCCACAAACAAGTCTCAATAAATGTAAAAAAGTTAAAATCCTACAA |
| | ATTATGTTCTCTCACCACAACAGAATTAAATTAGAATTAACTTTAGAAAGAAATATAGGAA |
| | ATCCCCAAATATTTGGAAATAAGTTATTTCAAAAGAACCCATGGTCAAAGAAGAAATCAAA |
| | CCAAAAAAATTAGACAATATCTTAAAATTAATGGTAACAAAAAATAACATCAAAACTCATG |
| | AAATAGGGCAAAGTCAGTGGGAAGATGGAAATTTACACCTTTAAAAGCTTGTATTA |
| SEQ ID NO: 176 | CCCCCTTTGCCTTTTTTTTTTTGAGATGGAGTCTGGCTCTATTGCCCAGGCTTGAGTGCAGTG |
| | GCACCATCCTGCCTCACTGCAACCTCTGCCTCCTGGGTTCAAGCGATTCTCCTGCCTCAGCCT |
| | CCCAAGTAGCTGGGACTACAGACACCTACAACCACACCAGCTAATTTTTGTATTTTTAGTAG |
| | AGACGAGGCTTCACCATGTTGGCCAGGCTGGTCTTGAACTCCTGACCTCAGGTGATCCGCCC |
| | ACCTTGGCCTCCCAAAGTGTTGGGAGGCCACTGCACCCGGCCTACCTTTG |
| SEQ ID NO: 177 | CATGATATATGTTTCAAAAAGAATTAGCATAGAAATCCTGGTCTCCTAGCCAAAAAAATCA |
| | AAGGATTTTCAAAAAAACGAATCTGTATGTTGAGGCAAAAGGATTGAACCTGGAAGTCTGG |
| | GACTTTATCATAGAAACAAAGTCTCAGATATTTTAGTTCTTTGGAAACAAATGCTGTAATTC |
| | AAAAGCATTTGACCTGTCACTGTACTATCTACATGTGGAAGAATGTTCAAGTTGAATCCTAA |
| | TGCCGTGAATGAAACACAGTCTGTGTAGGGAATGAGCAAAAAAGTTGAATTCCA |
| SEQ ID NO: 178 | AGACACAATGGAGTTACATAAAATGCTCAATTAAAACCAGAAAAGTCAGAAAAATAGTAG |
| | AAGAAAAAAAAAGAAAAAACCCATGACAAAAAGCTGTTATAATTAAGGTAGCTATCCAAT |
| | CAATAATATCAATATCACTTTAAACGTAAATGGTCTATCAGTTAAAAGAGACTATCAAAGT |
| | GGATTTAAAAAAAAGCAAGACCTGATAGAATGTTTTCCACAAAAAAATCACTTCACAAGCA |
| | TGTATCTGATAGACTCATATCCAAATTATACAAAGCATTCCTAAAATTTGACAATGAA |
| SEQ ID NO: 179 | GCAAGGGCTCTTGTGGCAAGAAGCTTACATCAGAGTGCAGAAGACAGAAAGTAAACAATA |
| | AACATAGTAAATTAGTCAGTTTTCTAGATTGTCAGAGGTGATAAACTCTATGAGAAAAGAA |
| | AGTAGAAAAGGTAAGGGGGTTAGGAATGCTGGGGGAGGAACAGATTACCACATTAATCAG |
| | GATGGTCCATATTAAATGGTATGCCCTCATTAAAGAGGTGAGATTTGAGCAGTGACTTAAA |
| | ATATAATGTAAACCAGCAGAAGATGGAAGAGAAAACTAAAATATTAAAAGTGAAAATA |
| SEQ ID NO: 180 | AAACTAGCAACAGAGAAAGCTAAAATCCAACATGTTTTGTGTTGCAGAATCCTCCAAAAGC |
| | CTCAATCTCTGTAGAAACAAGGAAGAGGCGAAGGAGACTCTGAGGAAGAGGATTGATTTTA |
| | AAAAGTCTAGTAGTCGGAGCCCCTTCCCAACCTCACACAGCTGGGCATCTGCTCCTCTGTCA |
| | GTACAATGAAAGAATAAAGTCATATTCCCTGGAGTTAATAAAGCAGAGGGTCTCCGTACAG |
| | GGAGTGAGTGTTAAGGTCAGGGGTTCTATACACTGAACTTAGAAAGCGTACGTGC |
| SEQ ID NO: 181 | GCAGAGGGTGGGGCGGAAGAGCAGACGGGGACGGGAAAGGCGCTGTCGGTGACATCACAG |
| | ATAGGGCGATTCCTATGCAGAGGAGGCAGCTCAGGGGCTGCTGCTTCGCCAGGAAAGATTT |
| | CTCGTGCTGTGGGAGCTAGTCCAGGACTTCCGGTTGGACGTGATAGTCCCAGCTGTGTGTCA |
| | GGGCTAGGAGGACTTGAGGCGGCATGGGGGCGGGGTGGGGGAATGCGCGGGGCAAGTGAC |
| | CGTACGTGTAAGGGGTGAGGCGTATGGAGCTGTGGCAGGGCGGAGGTGCGTTCATTC |
| SEQ ID NO: 182 | TACCAAAGATGATGAAAATAAGTATATGTACAAAATATTTTAGTATTTATGTGCCTGTAAAT |
| | ACAAAAGGAGCAATAAAAGTGATTTCATTTCAGAAGGTGAACATTTTGAAAGAAATAATAT |
| | TCATGTAAATTCTGAACTAAAATAGAATGAAATAAAATTCTGAAATAAGATAAAAATAGAA |
| | TGTTAGCATTATAGGAAACTATGGAGATTATTTGAGCTAATCTTCTCATTTTATGTATATGGA |
| | AGCTGAGAAGTGACATATCCATAGTCATACAGCTAATAAATAATCAGGATGGA |
| SEQ ID NO: 183 | GCGTTGGGTGAGGCGGAAGAGCAGACGGGGATCCAGAAGGCGTTGTCGGTGACATCACGG |
| | AGAGGGCGATTTCTATGTAGATGAGGCAGCGCAGGGGCTGCTGCTTCGCCACCTGCTGCTTC |
| | GCCACGAAAGAGTTCCCGTGCCGTGGGAGTAAGTCTGGGACCTCTGGTCGGACCGGAGAGT |
| | CGCAGCTGTGTGTTAGGGCTAGGATGGCTCCTGGATGCGCGTGACGCAAGTGACCTTGCGT |
| | GTAAAGGGTGAGGCATATGAGGCTGCGGCGGGGCGGAGGGGCGTGAGCTTATACTT |
| SEQ ID NO: 184 | CGGAGGGTGGGGCGGAAGAGCAGACGGTGTCTGGGAAAGGCGCTGTCGGTGACATCACGG |
| | ATAGGGCGATTTCTATGTAGATGAGGCAGCGCAGGGGCTGCTGCTTCGCCACTAAGGAGTT |
| | CCCGTGCCGTGGGAGCGGGTTCAGGACCGCTGGTCGGACCTGAGAGTCCCAGCTATGTGTC |
| | AGGGCTAGGAGGGCTGGGGGCGGGGGGGGTGGGGGGGGGGGGCGTGCGCGGGGCAAGTG |
| | ACCGTGCGTGTAAAGGGTGAAGCGTGTGAGGCTGTGGCGGGGCGGAGGTGCAAAAGCTC |
| SEQ ID NO: 185 | GGGTTTGGAAGAACCCCGCGTCCACTGTAAGCTCAGGGGAGAGCGGGAGCCAGGGAGGTG |
| | AAGTGCACAGACTGGACAGAGGCGGCGGGCAGAACCGCGGGGGTGAGAGGGCGCGTGGTT |
| | GCGGGGCGGGAGCCACTGCTGAAAGGCGGCCTGGGTTGTCGTGTGGGGTGACTGTCGGTGG |
| | AATCTTTGGCAGAGAGTGGTTTGGAAGAATGGCGAGGGGGGCAGTGGGTAGGGTGGTGAC |
| | CCTGAGCGTCCGACCAGGGCGAGGACGCTGTGCTGTCCCTGCAGGGCATGCGCTCATTC |
| SEQ ID NO: 186 | GTGGGGCGGAAGAGCAGACGGGGACTGGGAAAGGCGCTGTCGGTGACATCACGGATAGGG |
| | CGATTTCTATGTAGATGAGGCAGCGCAGGGGCTGCTGCTTCGCCACTAAGGAGTTCCCGTGC |
| | CGTGGGAGCGGGTTCAGGACCGCTGGTCGGACCTGAGAGTCCCAGCTGTGTGTCAGGGCTA |
| | GGAGGGCTGTGGGCGGTGGGGGGGTGGGGTGGGGGGGGGGGGTTCGCGGGGCAAGTGAC |
| | CGTGCGTGTAAAGGGTGAAGCGTGTGAGGCTGTGGCGGGGCGGAGGTGCAAAAGCTC |
| SEQ ID NO: 187 | GTAGGCTGAGCGGCAGAAAGGCAGACGGGGACTGGGAAAGGCACTGTCGGTGACATCACG |
| | GATAGGGCGACTTCTATGTAAATGAGGCAGCGCAGGGGCTGCTGCTTCGCCACGAAGGATT |
| | TCCCGTGCCGTGGGAGCGGGTTCAGGACCGCTGGTCGGACCTGAGAGTCCCAGCTGTGTGT |
| | GAGGGCTAGGAGGGCTGGGGGTGGGGGGGGGTGGGGGGGGGGGGTGCGCGGGGCAAGTG |
| | ACCGTGCGTGTAAAGGGTGAAGCGTGTGAGGCTGTGGCGGGGCGGAGGTGCAAAAGCTC |
| SEQ ID NO: 188 | GCAGAGGGTGGGGCGGAAGAGCAGACGGGGACGGGAAAGGCGCTGTCGGTGACATCACAG |
| | ATAGGGCGATTCCTATGCAGAGGAGGCAGCTCAGGGGCTGCTGCTTCGCCACGAAAGATTT |
| | CTCGTGCTGTGGGAGCTAGTCCAGGACCTCCGGTTGGACGTGATAGTCCCAGCTGTGTGTCA |
| | GGGCTAGGAGGACTTGAGGCGGCATGGGGGCGGGGTGGGGGAATGCGCGGGGCAAGTGAC |
| | CGTGCGTGTAAGGGGTGAGGCGTATGGAGCTGTGGCAGGGCGGAGGTGCGTTCATTC |
| SEQ ID NO: 189 | TCCCAAGAGGGGTTCGGAGGAACCCCGCGTCCACTGTAAGCTCAGGGGGGAGCCGGAGCC |
| | AGGGAGGTGAAGTGCACAGACTGGACAGAGGCGGCGGGCAGAACCGCGGGGGTGAGAGG |
| | GCGCGGTGGCTGCGGAGCGGGAGCCGCTGTTGAAAGGAGGCCTGGGTTGTCCTGTGGGTGA |
| | CTGTTGGTGGAATCTTTCGCGGAAAGCGTTTTGGAAGAATGGCGCGACGAGCGAGCAGAGG |
| | GGAAGGTGGTGACCCTGAGCGCTCGGCTAGGGGAGAGGAGGCTGTGCTGTTTCTCCTCT |
| SEQ ID NO: 190 | GGCTGAGTGGCGGAAGATGGGGGGGGAAAGTTGACGGAGACGGGAAAGGCGCTGTCGGTG |
| | ACATCACGGATAGGGCGACTTCTATGTAGATGAGGCAGCGCAGGGGCCGCTGCTTCTCCAC |
| | CTGCTGCTTCGCCACGAAGGAGTTCCGTGCTGTGGGAGCGAGTCCAGGACAGCTGGTCGGA |
| | CCTCAGAGTCCCAGCTGTGTGTCAGGGCTAGGAGGGCTCGGGGACGCGCGGGGCAAGTGAC |
| | CGTGCGTGTAAAGGTTGAGGCGTATGGAGCTGTGGCGGGGCGGAGGTGTGCAAATCC |
| SEQ ID NO: 191 | TAGGTCCGCTGAGGGGCGTTGGGTGAGGCGGAAGAGCAGACGGGGATCCGGAAGGCGTTG |
| | TCGGTGACATCACGGAGAGGGCGATTTCTATGTAGATGAGGCAGCGCAGGGGCTGCTGCTT |
| | TGCCACGAAAGAGTTCCCGTGCCGTGGGAGCAAGTCTGGGACCGCTGGTCGGACCGGAGAG |
| | TCGCAGCTGTGTGTTAGGGCTAGGATGGCTCCGGGATGCGCGTGACGCAAGTGACCTTGCG |
| | TGTAAAGGGTGAGGAATATGAGGCTGCGGCGGGGCGGAGGGGTGTGAGCTTATACTT |
| SEQ ID NO: 192 | GGGCGGAAGAGCAGACGGGGACTGGGAAAGGCGCTGTCGGTGACATCACGGATAGGGCGA |
| | TTTCTATGTAGATGAGGCAGCGCAGGGGCTGCTGCTTCGCCACTAAGGAGTTCCCGTGCCGT |
| | GGGAGTGGGTTCAGGACCGCTGGTCGGACCTGAGAGTCCCAGCTGTGTGTCAGGGCTAGGA |
| | GGGCTGTGGGTGGTGGGGGGGGGGGGTGGGGGGGGGGGGGCGTGCGCGGGGCAAGTGACC |
| | GTGCGTGTAAAGGGTGAAGCGTGTGAGGCTGTGGCGGGGCGGAGAGTGCAAGAGTTC |
| SEQ ID NO: 193 | GCGTTGGGTGAGGCGGAAGAGCAGACGGGGATCCGGAAGGCGTTGTCGGTGACATCACGG |
| | AGAGGGCGATTTCTATGTAGATGAGGCAGCGCAGGGGCTGCTGCTTCGCCACCTGCTGCTTC |
| | GCCACGAAAGAGTTCCCGTGCCGTGGGAGCAAGTCTGGGACCTCTGGTCGGAACGGAGAGT |
| | CGCAGCTGTGTGTTAGGGCTAGGATGGCTCCGGGATGCGCGTGAGGCAAGTGACCTTGCGT |
| | GTAAAGGGTGAGGAATATGAGGCTGCGGCGGGGCGGAGGGGCGTGAGCTTATACTT |
| SEQ ID NO: 194 | CAGAGCTAGAGCCGAGATTTTAAAGATGGTAGGTTAACACCATAAAAGACAACAATTTTGA |
| | AAGCAGTTGGGTGAAAGCAGTATGTAAGTTAGTAATATTTAAATAAAACTATCCTGGAGGA |
| | ATTGTTACTGAGTTAATTGTTGCTGTAATAAACACTAAGACCTTGGAGGAAAAACGACGCT |
| | GTCCTAATGAAAATCAGACATTAATTAGCATAGAAATGGCACCGCGATGCCGCTCTAATTTC |
| | CCTTTGGTTGGTTTCCATGGAAATCTGTAGGTAAAGTGTGCTTTTAAAAGTGTTT |
| SEQ ID NO: 195 | CGAGGTCAGGAATTCGAGACCAGCCTGGCCAACATGTTGAAACCCCGTCTCTACTAAAAAT |
| | ACAAAAATTAGCCAGGTGTGGTGGTGGTTGCCTGTAATCCCAGCTACTTGGGAGGCTAAGG |
| | TAGGAGAATCGCTTGAAACCGGAAGGTGGAGGTAGCAGTGAGCCAAGATCACGCCACTGC |
| | ACTCTAGCCTGGGCAACGAGCGAAACTGTGTCTCAAAAAACAACGAAACAAACAAACAAA |
| | CAAAAAAGTTTTTGAAGAAAGTCAAAAGAATGTATTCTGTATTCTAAAAATGCATTCT |
| SEQ ID NO: 196 | TGGAGTCTCGCTCTGTCGTACAAGCTGGAGTGCTGTGGCGCAATCTTGGCTCGCTGCAACCT |
| | CCACCTCCCGGGTTCAAGTGATTCTCCTGCCTCAGCCTCCCGAGTAACTGGGATTACAGGCG |
| | CGTACCACCATGCCTGGCTAATTTTTGTTGTATTTTTAGTAGAGATGGGGTTTCACCATGTTG |
| | GTCAGGCTGATCTTGAATCCCTGACCTTGTGATCTGTCCACCTCAGCCTTCCAAATTGCTGG |
| | GATTACAGGCGTGAGCCCTGTGCCTGGCATATGTTTAAAAGTTGATTGGAC |
| SEQ ID NO: 197 | ACAAGCAATGGGGAAAGGATTCCCTATTCAATACATGATCCTGGAATAACATATGCAGAAG |
| | ATTAAAACTGGACCCCTTTATTTCACTATGTATAAAAAATCAACTCAAGATGGATTAAAGAA |
| | TAAAATATAAAACTTAAACTATAAAATCCCTAGAAGAAAGCATAGGAAATACCATTCTGGA |
| | CATAAGAACTGGCAAAGATTTCATAACAAGGACACCAAAAGTCATTGCAACAAAAACAAA |
| | AATTGACAGGTGAGACCTAATTAAACTTAAGTGCTTCTGCATGGCATAAGAAACTA |
| SEQ ID NO: 198 | TTTTATAAGGAGTAAACAAAGCTAGAAAGAACCAGGTATGGGGAAGTGAGGCAAACGGGT |
| | GACGTGATCAGATAGATCAGAGACTATTTTACCTGGAGGCCAGTTTATTCTCCGGAGGGGCT |
| | GTATGCTTTTTCAGGATAATGGTGGGGCAAAATTTAGGGGTCTGGAGGAAGGAGAGAGCTT |
| | AACCAAAATTTTATTAAGGGGCATTTTGTTCCAATTGATCAAAATTTTCTTCTTTCTGTACAG |
| | AGGCTACAGTTGCCTCATGGTCTGTATGTCCTGGTCATCAGAAAACATTATTCA |
| SEQ ID NO: 199 | ACCCTAAAGACCGTCGGGTGGGGGCTGAGGGCGAGGGGGGGGACACCGGGGCCGCGGGCG |
| | GGGCGCACCCGGAACCCCGACAGCTGTGTCTTGGTGGAGCTGTGGACTGCGCCCGCCGACT |
| | CCCACGGCCGGGGCGGCGCTGAAGCAGAGAGAGGCCTGGGTGGGAGAGCCGGCCCCGGGC |
| | AGGGTGCGGGCGTTGAAAAGTGCGTGTCTGCAGGTGCCAACCCGGGCTGTGAAGACTCATC |
| | TCCTGGAGGGTTCCTAATGTCATGCTAGAGGGCTGACGGAGATACAGAAGCTCATCGC |
| SEQ ID NO: 200 | CCTCTAATATTTCTGAAGCAGTGTCTGGCATGTAGGAAACTCTATGTGAGCAGTTGTTAAAT |
| | ATATAAGTGTTTGAATCTCCTGAGTGTTTCAATTGTTCAGCAAATTTATAATCCACTTACGTT |
| | TACTTTCACCATCTTCTGCAATGAATATTATGAGAAACTCCACATGCTTTGCAGAAATCTAT |
| | TTACATGGTGTCTGGAGTTTTTGATCAACATAGCAGTCACTTAATAAAGGCAGTGAAGTTAG |
| | TTTGAAATCACTTTATGAACAGTAAAATATTTGTTATTAATATTATTACAA |
| SEQ ID NO: 201 | CTCACTGCTGCACAGTTGGCCCCCTGTCTCTGTGGGTTCTGCATTTGTAAATTCAACCAACTG |
| | CAAATCGAAAATATTTGGGAAAAAAATTCATCTGTACTGAACATGTACAGACTTTTTTCTTG |
| | TCGTTATTCCCTAAACAATACAGTATAACAACTATTTACATAGCATTTACAGTGTATTAGGT |
| | ATTATAAGTAATCCAGAGATGATTTAAAGTATACAGGCCTATGGATGTAGGTTATATGCAA |
| | ATACTATGCCATTTTATATCAGGGACTTGAGCACCTTCAGATATTGGTATCT |
| SEQ ID NO: 202 | AGGCGGGAGGATCTCTAGAGCCCAGGAGTTCAAGACCAGCCTGGGCCACATACTGAGACCT |
| | CATCTCTACTAAAAAAGTTTAAAAATTAGCCAGATATGGTAGCACACACCTGTAGTCCCAG |
| | CTAATCGGGCGGCTGAGTTGGGAAGATCGCTTGAGCACAGGATGTCAAGGCTGCAGTGAGC |
| | TATGATCATGCCACTACACTCCAGTCTGGGTGACAGAGCAAGACCCTTTCTCTAAAAAATAA |
| | AAATAAATAAATAACATAAATAAATAAATAAATAACAAAAATAAATAAAAATCTA |
| SEQ ID NO: 203 | TATGAATTGTTTAGAGCTGCACTGTCTGACATGGTAGCCATTAACCACGTGTGGTCATCGAG |
| | TGCTTGACACGTGGCCAGTAGAAATTAAGATGCGCTGTGTATGTAAAATACACACCCAATTT |
| | CAAAAACTCAGTACAAGTGAAAATGTAAGCTATTTTCGTAATTTTTGTATAATGATTACATG |
| | TGAAATGATAATAGCTTATATAATATTAGGTTAAAGAAAGCATTAGAATTAATTTCATCTGT |
| | CTCTTTTCACCTCTTAATGTGGTTACTAAAAAATTTTAAATTACATATGTGA |
| SEQ ID NO: 204 | AATGGGGACAGGAAATAAAGCAAAGCCTCAGAGTCCTCCTCCCAAACACAGGAGAGCACC |
| | AGGGGCTTTTTTTCTTAGAGACTGTGCCCCTCCTCTGATTACACACCTTCTCTCTACCCTCCA |
| | TGCATGAAGTTCCCCACTGTTTTCACTCTTCTTTCCTCGTGGCATACATTATGATTTCACTTTT |
| | GGTGGCCAATAATAGTTCACTCTCTATATATTTGTTGAGCACTTACTATGAACTGGACACTC |
| | TTCTAGATGCTGGGAATGCAACAGTGAACAAAGACAAAAACCCTTGCCTTC |
| SEQ ID NO: 205 | TGAACTTAAAAGTTAAAAAAACACAAAAATAAAATAGGGGTTATAAGCAAAATGTGTGCA |
| | ACAGGTGGTACATGTGTAAAGACTGTCAAGATTGGGTTGAAAAGTCTGTGAGAGTGCCTTT |
| | GCAGCTTGCTTCTCTGAGCCTTAAGGTGTTCCACCTCTAAAATTGGTTGAAATGAGGTATTG |
| | TATGGAAAAGGCTTTGGTAAACGAGGAAGTGCTATACAAATGTCAAATATTAGTTTTTTCAT |
| | TAGTTATATTGTTATTTTATGTCACTGTTATAACATTGACATTCTGTTCAGTGTT |
| SEQ ID NO: 206 | ATAGAGAAACACAAGATACAAACCCAAAGTGAAGGAGTGAAAGAGTGAGGGACATAACAT |
| | TATGTAGAGATGTGAACTGGACCACTTGTGAAGAGATGGTTTTTGATACAAGAAACACTTC |
| | CCCCACTGTATTACATTGAGCAATATGAAACTGGCATGTTCAACCATTTTTGAAATATAAAA |
| | GTAAAAATTTCATATAGTTCAATGTTATGGTAGAAAAGAAACAAGAGAAAGCAGCTACAAA |
| | TGCACTTAGATTTATAGATTTAGTGGTGGAAATTTAAAGAAGCACCTGTCTCTCCA |
| SEQ ID NO: 207 | TGCTCTTCTAAGAATTCTTACCACTGGGACATTTTGTTGACATAAGGCACTGTGTCTCTATGA |
| | AACAAAATGGGACTCTAGCAGAGAAAACATATCAAATTACTTGAAAGATTCTTGAGTATTG |
| | ATTTATATCAGGGGTTGGCAAACATTCTCTAATGGGCCACATAGTAAATATTTTAAGCTTTG |
| | AAGGCCATATGATCTCTGCTGCAACTTCTCAACTTGGCCATTATAGGGTGAAAGTAGCCATA |
| | GACAATACATAAATCAATGTGTGTGGCTGTGTTCCAATAAAACTTTATTTAT |
| SEQ ID NO: 208 | GAGGCAGGAGAATAGCTGGAACCCGGAGGCGGAGGTTGCAGTGAGCCAAGATTGTACCAC |
| | TGCACCCTAGCCTGGGCAACAGCGAGATTCAGTCTCAAAAAAAAAAAAAAAATAATAATA |
| | ATAAATAAATAAATAAATAAATAAAATTTAACCAGGCATGGTGGTGTGTGCCTGCAGTCCC |
| | AGCTACCTGGGAGGCTAAGGTAGGAAGGTCCCTTGAGCACAGGAGTTTGAGGCTGCAGCGG |
| | GCCATGTAACGCCATTGCACTGCAGCCTGGGTGACAGAGTGCGACCCTGTCTCGAAGG |
| SEQ ID NO: 209 | CCTGAGATCCTGCCTTCTCCAGCAGTGCTTAGTGAATAAGGTACGATTCTGTGTACCCAGAG |
| | CCACTGGGCTGCCCAACACACAGTCATAATTAGCTTCCTTGTGGGGTGCCTAGAATACAGG |
| | GATGAGTGAGGGCACAGATCCTGCCTGCCGGAGCTGAGAGAATTTCATTTCCCTGTAGTGA |
| | CAGAGTGTGTGTCCAGGGAGTTGCAGGATTTCCATGGCCACCCCTAGGCTCTCCTGACCTGT |
| | TCAACAGTGACCATTCCTTCTGAAATATTATTTCACTTATAAAAAGCTTCCTAC |
| SEQ ID NO: 210 | GTATATAATACATATAACATGCAAAATTTGTGTTGTTTATATTATTTGTCAAAGCTTCTGGTC |
| | AACAGTAGGCTGTTAGTATTTAAGTTTTGGGGAAGACAAAAGTTATATATGGATTTTCAACC |
| | TCATTGGAGGGTTGACACCTCTGACCCCCATGCGGTTCAAGGGTCAACTGTACTCTCTATTT |
| | TAATTGGGATAATGGTTACAGATGTGTATGTATTTGTCAAAACTGACCAAATTGTTCACTTA |
| | AAATATATGCATTTTATTGTTTGCAAATTATACTGTAATAAAAGTCAAACA |
| SEQ ID NO: 211 | AGGCTGCACTGTCCCAGGGGCACCCAGCCTCCAAATGAGCCTCAAGGAAGGGTCTGGAAGG |
| | CCAGACAATACTATGGTCAGATTATGGACTTGGGAATTGCTGCAGATGCCATTGGGGGTAA |
| | GGAAGATTAGGAAATTCTGGAACTCAGGAGCTGCCCAGGCAGCCAGACTGCCTCTGACACA |
| | CCCAGTCCTCATTTGGATTTGAGGAAAGGAGACTTGAGCGCAGGAGAAAGGCACCCAGGAG |
| | CCTGTGAGTCGTGCCTAAGCCTCTGGATCCCACATCTGAGGAAACAACTTAATGTA |
| SEQ ID NO: 212 | AGATCGAAAAGAAGTCATTCATGAAATGAAATTTTCTCCAGATGGTTCTTACCTTGCAGTGG |
| | GATCCAATGATGGCCCAGTAGATGTCTATGCTGTTGCCCAGAGGTATAAGAAAATTGGAGA |
| | ATGCAGCAAGTCCCTTAGTTTCATCACGCATATTGACTGGTCCTTGGATAGTAAATACTTAC |
| | AAACTAATGACGGTGCAGGAGAACGATTGTTCTACAGAATGCCATGTAAGTCATGTGGAGG |
| | CCTTGGATGTTTCTGGAAAGCAAAATTTTGAACCAGGTGAAGGAAATACAGGAC |
| SEQ ID NO: 213 | TGCCTACCATATGCCTAACAAAGAGAAATAGGAGATAGGCTTCCCGTCCTTAGAGAGTTTA |
| | TGACATCTCCGAAGAGACAAGATCAAGTGCGAAAGACAGATTGGAACACTACAAGAAGGA |
| | ACCTAGCAAAGCTCCGAATTGCTTCTTGGGCAGAACCGAGTACCTGATAGACCATTGGGTA |
| | CTGGGGTCACCTGGTAGGAAGCAGAGAGAGAATCATTTACCATGGAGTTACATGGTAGTTA |
| | CCAGAAATTTGTTTTGTTTTCTTTTTTAAGTTGTTGTTAAAAATATTTTTCTCAGAT |
| SEQ ID NO: 214 | TGATATGTATCTATTAAAATGCCTACAGCAAGCATTATCCTAAATAAGAGAAATATCAGAA |
| | ACATTCGCTTTGAAGTTAAGAATGAGAAACCAAGGCACACTTTTACCACTTGTATTCAACAT |
| | CGTAGTGAAGGTCATAGCCAGAGCAGTGAGCATTGTAGCCAGTGTACAGTAAGACCCCCCA |
| | AAAAGTAGAGGCATAGGATTGAAAAATGAGGAAATAAAACTATAATTATTTGAAATTATTA |
| | TAGTGATAACCCAAAAGATATATAAATGGATTATTTAGATAAGTTTAAAAGAGTG |
| SEQ ID NO: 215 | AATTTGTAATACATCAGGGGTTTCAATAATGGAATTCACAGCCAGCTGACAAACAGGTTTTT |
| | ATTAATGACACAAGTTTTCAATGAAGAATTTTAGCAAAACAACTATTTTAAGCAATTGCATT |
| | TCCAAACAAAAAAAAGATGCACAGCAGTATGTATAAGCCTCTTGAATTATAGAATTATTTA |
| | TATTAGCAAATAAAAAAACTAACCTAGATGTCTAATAAGAAGTAGTAAAAGGCTAATAAAA |
| | TAGCAAAATGTTTACAATATTGTTGAATGAAAATCATTTCAAAAAATAGATCTA |
| SEQ ID NO: 216 | AAACAAAACACAGTAATGGAGATGAGATGCGCTTTTGATGGGCCCATCAGTATAATTTGTA |
| | GAGCTGGGTAGAGAATAAGTAAACTTAAAGATATTACCCAAAATAAAATGCAAAGAGGAA |
| | AAAAGCAAAAAAAATCTCAAGACAGTACAGATAATCCAAGAACTGTAGGACAATATCAGA |
| | AATATCAGACAGTCTAACATAAGCATAATTAGAATTCCAGAAGAAGAAGAAAGATAAAATT |
| | AGGCAGAAAACGTATCTGAGAAAAATAATGGCTGAGAACTTTTAAAAATTAGTGACAG |
| SEQ ID NO: 217 | TGCCAAATAATAACAAAATATAATAAAAAATATAAAAAGAAAAAGAAACACCAGATGGAT |
| | TTCCCATTCTTGAAATGAACCACTATTAACAGTTGTGTGATTTTCTTTGTTTCTAATCTTCAA |
| | AACAAACATATACACCCATACAATTACACATATACATATAATGAACAGTGCATTTTTTCCCT |
| | TCAATTTCAGTTTAACATATAAATCTACTATCTCTCTATCTATCTATCTATCTATCTATCTATC |
| | TATCTATCTATCTGTGTATCAATCATCATTTATCTATTTATCTAACATCAC |
| SEQ ID NO: 218 | TAGCTGCGGGTGGAGTTCAAGAGCGCGCCGCGGTCCCGCCCCCGCCAGCCCCGCCCCGGCG |
| | AGAAAAGCGTATGCAAATTTTCGAGCGGCCGACGCGCGGTCTTCTGGGTAAAACCGAGCCG |
| | CCGCTTTTGCGACCCTTCGGGAGCCTCAGAAAACAAAGAATTGGGGTGTCGTCGAAAGCTG |
| | TGGAGGCTGGAGGTAAGCTAGCTACCAGACCGACTAGGGCGAGGCTCACGAATTAATTACC |
| | ACAACCCTACCAGGTATTGGCGCTTCCTGCTTGCAGCCCAGGGACTTTCTATTATA |
| SEQ ID NO: 219 | AAAAAATTGACGCCTGAGTACTTTGGGTGCCTTTAAAATGTTTGCCTCCAGTAGAGTGCCTC |
| | ATTCACCTCACTCTATCCCGCCTGGTTGCAGAGCCACGGTAGTGAATGGTTTACCCTCAGAT |
| | AGACAGTGGGGTCCCACCAAGCCGTTTCCTGCTGTCCCACTGCAAGATGCATGACAAACCG |
| | CCCTGCACCTGAGGTTATTAAGACAGACTTGGGAGAGAAGACGAGACCAGGCAGAGAGAC |
| | AAAGACAGAAAAAGAGACTGAGGAAAACTGAAGGACAAAGGTGGAAAGAAGTGGA |
| SEQ ID NO: 220 | CAGAAAGAACCTTTGGGCCCAGGACCCTGGGTTTTGTAAGAACCAGTCTTTGGGAGAACAG |
| | ACCTAAACCCTCTTCTTCTAAAGTGAGTGGTGTTTTTACTAGTTGCTTCTTCAAACTTTGTAA |
| | TTCGAGTCCTTCTTTAAAGTAACAAAAATGTTTTTAAAGTCTTCTGCTGAATCTCTTACCAAA |
| | CAATAACCTTCAAGTTTTGGTGAAACCATTACTTTTATATGTTGAGCTTGCTTAAAACAAGG |
| | CACGCAAGTAATCCTGTGACATACTCCATTTTCTTCAAAAACTGATGAACA |
| SEQ ID NO: 221 | AAATCCAATTCTGATCAAACTTGTTATAAGTTGTCTTGCAAAAAATGTTATCACATTTGGTA |
| | ATCCTACACTTTTGTAATTTAAAAACATAGAGTTTGCAAACTCATGGTCATGATTGTTTTGTT |
| | TGTCCCACATAATCTTTTTCCAAAGTCTATTTTACTTACCGTCTTTAAAAAATAAGAGAGTTC |
| | AGAAGATAATTTTAAATTCCAGATTTCTAGGAAAAAAAGAGGGAGAGGGGCTAATTTGACA |
| | GTGCACAGTTTGAATTTCTACTTAGCAATATTCTTCCAAAGCACAGGGGCT |
| SEQ ID NO: 222 | AATGTTGTGTTTTTGTTGTTTGTACAATTTTACTAGTGCCTAGTTCACAATCCAAATACAAAT |
| | CTAGGTGATTTTTGAATAATAGATGATACTTGTTAAACTCTTACTATATCACTACTGTTCCAA |
| | GCACTGTGAGATATTTTCCCATTTAGGCATCTAACAGTTCTGTGAAATTAATACTGTGAAGC |
| | ACAGGGAGATTAAATGACTTCTGACAGGACACATAGCAAATAAGTAATGGAGCTGGGATTC |
| | GCGCTCAAGAAATATGACTCCATGGCCTCACTTAAGTTCACACACTGTGAT |
| SEQ ID NO: 223 | TACGAATGATTGCATCTTCCTCTTTAAACTGTTTTGTTTTTTAATAGAAAAAACTTCAATTTT |
| | GCCAACTCTTCCCCATTTTAATTGTTAGTTTGAGCTCCTTCTTTCACATCCTCCATTTGACAA |
| | ATTAGTATCGACATCTTGCAAAGTCTGATGATACCTTTTTAACTTTTTTCTTCTTTTTTATATA |
| | CAAGTCAAGTCTTCTGGGGTTTTTTATTGGTAGTTTTCTATTATTCTATTTTAGTAGAGACTA |
| | TGTTATTTTAATTTTTACTATTATATATGTGTGTGTGTGTGTGTGTA |
| SEQ ID NO: 224 | TTTTTACCAACCCTGAATTACCATCAGCTCCCTGTGAGTTTGGTTAGCACTATCCATGGGGTC |
| | CCTTTGGGAAGAGTGGTCCCAGGGGGACTACTGATAAAATTCAACTGAGGTTCTCAGTGGG |
| | CAGGCAGGCTAAAGCTCAAATCCTTTGTGGTCAAGATACAGGCACACTGTGAGGTCCGGTC |
| | CCAAACAATCTATTGGCTTCTCCCTCTTTTTGACATCCTAACTGTGGCACAAGGAATGTCCTC |
| | TTTAAATGAGTTTAAAAGGCAATTGAAATGAACATAAATCAAATGAAATGCT |
| SEQ ID NO: 225 | ATTTGTTTTTTTACTGAGCTAATTTTAATGATTTCAGCAAAAGTTATTTTGTTATTGAGTGAT |
| | GAAACAAAAGATTTTTGAGGGTGTTCATGAGCATAAGTGTATCATATAGTCACTTATGGTTG |
| | TTGTGTTCCTAGTACTTAGTACAAGGTATCGGAATTGTTCTTTAGTTAATCTTTTATCTTGAG |
| | AAGTTAAATCTTGGTGTAATCTTATCCTCATGTTCATATAAAGATATGTGGAGTTTGTAGTG |
| | AGGACTTAGAGCCGGGTACAATTTCATGGTTAAATTAGAAATGCTTTTGG |
| SEQ ID NO: 226 | CCATAGGCATCCATACAACAGAAACTTCGATGGAAAATTTCACCATTCAAGATTTTAATTGA |
| | ACTGATGGCCAGGCCCTTCTCCGGTGGAAAAAAAATTAGATAAAATTAAAAATGCTTTCAT |
| | TTGGAGGTAAGCTTTAAAAAGGGGTGGAAGAAAAGGGGGATCCTCAAGTTTCAGGAATGAT |
| | TAGAAAATTCACAACCAGAGAACTTAGCACATGATGACACTGCATTGACCACACAGCCTAC |
| | AGAGCTGGAAATAAGACCTACAGTTCTGGGAATTGGGGTTCTAAAAATCACATGG |
| SEQ ID NO: 227 | ATATATTCTTTTTTTGAGACAGTGTCTCACTCTGTCGCCCAGCCTCCTGAGTAGCTGAGACCA |
| | CAGATGCGCGCCACCACACCCGGCTAATTTTTGTAGTTTTTGTAGAGACGGGGTTTCACCAT |
| | GTTAGCCAGGCTAGTCACGAAATCCTGGACTCAAGCAACGGGGGCAGATATATTCTTAATT |
| | GCTGTGTATTGAGTTAATCCTCTAACACTGTGGCACTTACCTAATGAGAATTATAAATTCAT |
| | AATTCTGCTAAGGGACTATCCTGAGGCTTAAAGCCTGAAAAATTCCCTCTGA |
| SEQ ID NO: 228 | ACATGCGACTCTTAATTTGGGACGGACAGAACAGCCGTACAGACCAGTAGTTCTCAGCGCC |
| | TTTGCTTACCCTGGGTTGCTCAGAAGACTTACTGGTTACTGGTTCCTTCTTCCCTTTTGAAGG |
| | ACTTAAAGGAGAAGAAGGAAGTTGTGGAAGAGGCAGAAAATGGAAGAGACGCCCCTGCTA |
| | ACGGGAATGCTGTGAGTGTCTGCTTTGCTCCTGAGCCCTGGCAGCTACCGCCCCACAAAATT |
| | TTTCCTGTTCTACTTTAAACATACCTATATATGTGTGTGTATGTGTATATGTAT |
| SEQ ID NO: 229 | TCTTCAGTCTGTCAGCCAATCCCAGAGGGAATCTTTTGGACTCTTGTGGCTTCTGGGTCCAC |
| | ATTTATTTTTGTTTTGGACAGAATACACTCAGAAAAAAAGAGAGACGGACTTAAAAGATGC |
| | ACTGTTCAAGTAAATGCCCAGAATTCAAATCGACATCAAGTTGGAAAACCGTCTCTTGTTAG |
| | GGACATAAATGAAAAGTAGATGGGTCTGTATGAGCGTGCAGGGAGTTTGGGAAGATAAAG |
| | TTTCTATTATCTAAACTGATAAAGCCTGGTATGTGTTTTATTCAATAATTAGAGG |
| SEQ ID NO: 230 | ATAAGACACCACTCAAAACAAAGCAAACACATTATAGGACAAAAGAAAACAATTCAAGAA |
| | ACAAGGCTAGAAGAACAGAACACTACCACTGTACCAGTTAAGGCTCCTGAATATAAACAAC |
| | AGACATATATTCTAGTGAATCTAAGTCAAAAAGGAATTTATTGGCTAGGTACTAGATAGTTC |
| | ACAACACTAAGACAAGGTTGGAAAATCCAGCTTGGACAGGAATCAAGGAAGGCAAATCAC |
| | AGAAGAATGTTTGCCTCCACAGCCTTGAATGAAGACTGGAACTTATGTTACTGAATC |
| SEQ ID NO: 231 | TTATAAAAAAGAATTAACACCAATTCTTCTCAAACTTTCAGAAAATTGAAGAGGAAGGAAT |
| | TCTTTTTAACTCATTCCATGAGCCCAGCATTACCTGGATACCAAAATCAGAAAACACACAAC |
| | AACAAAGAAGAAAACTATAGGCCAATATCCCTGATGAATGTAGATGTGAAAATCCTCAACA |
| | AAATACTAGCAAGCTGAATCAAACAACATATTTTTTTTTTTAAAAAAAGCACCATGATCAAG |
| | TGGGATTTATTCCAGGGATGCAAGGATGGTTCACACAGAAATCAATAAATGTGA |
| SEQ ID NO: 232 | AGAGGGACATTTGGATAAAGTGAAGAGCAGAACCTACCTATTAGGGAAGGGAAGTGGATT |
| | GGGTTTCCTACCCTGTCTGTAGACTCCATGAAGAACCATGTCTCTCCAGCCAGAAAGATATT |
| | CTTGCTTAAGTAGCTCCTTACAACCTACTTCTTCATAAATTTTGTAGATTCTTTCTTAAAAAT |
| | TGAAATATAATTTACACACCATAGAATTCATCCTTTTAAAATATATAGCTCAGGATTTGTGG |
| | TAAGTTCACAAGGTTATGCAATCTCCACCACTAATTAATTCAAGAATATTTTC |
| SEQ ID NO: 233 | AGCTCTCATAGTGATGGTGCATAGGCTAGTGGTGGCCCCTAAGACACCAATGCCAGTGGTT |
| | CACATCTGCTAGCCAGCTCGTCTCCAGAAAAAAGTACAGCACAGAAAATATCACAGTTCTA |
| | ATTGTTCCATACTCAGTAAGAACCATACTAACTACACATATTCAGATGAAAACAGTATTAGA |
| | ATACAGGTATTAGACTTCAGAGCTTTACTAAGCCCCATTCTAGAAGGTTGGACTAAAATACT |
| | AAAGATAAAAGGAGGACCAAAGTTAGGAAATTACAAGATATATAAGAAAAATTC |
| SEQ ID NO: 234 | TGCTGGCCTAATAGAATGAGTTTGGGAAGAGTCCCTCCTCAATTTTTTGGGATGGTTTCTGT |
| | AGGAATGGTACCAACTCTTCTTTGTACATCTGGTAGGATTTGGCTGAGAATCTATCAGGTCC |
| | CAGGCTTTTTTTGGTTGGTAGGCTGTTTATTACTGATCCAATTTTGGAGCTTGTTGTTAAGTC |
| | TGTTCAGGGAATCAGTTTCTTCCTGGCTCAGTCTTGGGAGAGTGTGTCCAGTAATTCATCTG |
| | TCTCTTCTAGGTTTTCTAGTTTGTGTGTTCGTTACTCTTAACAATAGATGG |
| SEQ ID NO: 235 | CCTTTTCCTTCCCATTCCCTCCTTTGGTGCCTTTCTCAGAGTACATGGACTTCCTTCTGCCAG |
| | ACTGGGGAGAGAAGTCTCCATCCCCAGCTCCTAGGGACCATGCAGCTGACCTGCTCCAAGG |
| | CACACTGGCAGCCCCAGCAAAATCCTGGAGCCGGCACCAGGGCATGTCCCACGAGACTGTT |
| | AGGAGGGCTGTGCATCTTTGCCCCTTGGTTGCTCATTGAGAAGCAGTATAGGGCTTCCATGC |
| | CTGTTTGGCCTCCCCTGGATCCCTGTAGCAGCTGTTAAAAGAGAACCTTTCCA |
| SEQ ID NO: 236 | CCCTGAAACGAGTGACCCCAAAACTGCCCAGCAGCCTGAGCATTCACTGTAATCCATAATA |
| | GTTCTTTAAAAAAATTAAAAATAAAAAAAATAGAGATGTGGTCTCACTATGCTGCCCAGGA |
| | CTCAAGCGATCCTCCTGTCTCAGTTTTCCAAAGTGCTGAGATTATAGGCATGAGCCATGGCA |
| | CCTGGCCCATAACTGTTCTTAACAAACCACCTTTGGAAGAAGTCAAGTGCTCTCTCTCCAGT |
| | TCCTTGAGAAGCACTTAGAATGCATAGTAAAGACCAAGTTTTAAGTAAGAGATC |
| SEQ ID NO: 237 | CGGGCAACAAAGACAGAGATGAGGTTACTTCTCATCTCATGCCTCTCAGAGATGAGGCATG |
| | TGTTACAAACATCAAACACTGAGCTTTGGGTAGTTGCTGCTGTTTTTTATTTGTTTTTCTGTTT |
| | GTTTTGTTTTTAATGTCAGGCACAGTGGTTCAAGCCTATAGTCCCTGGAGGCTGGAGGCTGA |
| | GGCAGGAGGATATTTGAAATCCCCGAGCCCAGGAATTCGAGGCTGCAGTGAGCTATGATCA |
| | TACCACAGCACTCAAGCCTGAGCAATATAGTGAGACCCTGTCTCTAAAAAAT |
| SEQ ID NO: 238 | TTTTCTACTGTAGATGGTTTGTAGACATTATTCCCACAGAGTGGGCTTCAGGATATTCATTTA |
| | TTCAATCACCAGATAATTATTGAGTGCCTACTATTTGCCAGACTTTGTATTTGTTTTATATAT |
| | ATGAATAAATTCTTGGTACTTGTGAGGTGTGTGTGTGTGTGTGTGTGTGTGTTTGTGTGTGTG |
| | AATAAATATGTGAGCAGACAGTAGATAAATAAATTATATATTATGTTACTGGATAGTAAGG |
| | GCAATGCAAAAATAAGAAAAAGGGTAAGGGAAATGAGGGTTAAGAGTGGA |
| SEQ ID NO: 239 | AGCCTAGTCTCCCCACGAGGAGGCGGCCCCGGGGGTGGAGTCAACCCTGGAGGCCACGCTC |
| | TGTGGGAAAGCACGGGGCATGCAAACTCGAAATGAAAGCCCGGGAACGCCGGAACAAGCA |
| | CAGGTGTAAGATTTCCCTTTTAAAACGTGGAGAATAAGAAATCAGCCCGAGTGTGTAATGG |
| | CGTCAATAGTGGTGTGGACGAGACAAAGGCAATGAGGCAAGGAGCGAGGCTGGGGCTCTC |
| | ACCGCGACTTTAATATGGATGAGAGTGGGACGGTGACGGCGGGGGCGAAAGCAACGGT |
| SEQ ID NO: 240 | CTCTGTGTCTTTGTTCTCACTAGAATTTGCTATTTAAAAATTTTTACATTAAAGTTTAATCAT |
| | ACAGTACATAGAACTCTTTGAGGACTAGAGTCTTTTTTTAAATTTTAGGGTTTGTCTTTTTTT |
| | TTTTTTTTTTATAAAAAAGAAGGTACTTCTCAAGTTTATGAGAAATACTGAGAGAGCTTTTG |
| | GTGACAATCCTACCTGAAAATTAAATACCTACACACACTCTCCTGCACCTGCCTTGATTTTT |
| | CACCATAGTATTATCAGCTTTTAACATATTATAAATTTAATTATTACATT |
| SEQ ID NO: 241 | TATTATAATATTATACAGTACTATAATATTATAGTATTATATAGTAGTAGATATATTAGATA |
| | CCATGAGGATATTATGAGTAGTGCTTATACTACTGTACTGTAGTGCTTATACTCAGGACTAG |
| | CTACATAATTTACAGGGCTCTGTGGTGGGGGTGGGGAAAGCAAGGCATCTTGTTAAAAAAT |
| | TATTAAGGATTTCATGATGCCACAGCAGAGCTTTTAGCCAAGTGCAGGGTCATTGTTAAGTG |
| | TAGGGCCCTGTGTGACTGCCCTGTTGTTTACATACTGTCTCAATATATACCCA |
| SEQ ID NO: 242 | CCTCTGGACAATTTCTTTTTTGTTTTGGGCCATGAAATAATTCATAGATTCACACCATTGCAA |
| | AAAATTCTTATGCATAGTGGAAATAATTGTTATGTGGCTTATGCAATTAGGAACTTTAGAAA |
| | GAGTGAAGTCATGCTGATATAAGAGGATAAGTATTTATTTGATATTAGGTTCAATTGATAGT |
| | CTATCTTCTTTCAATTTTTAAATTTTCTTTTTCTTTTTTTTCTTTCTTATTTGGCTATTGTTTCTA |
| | GGTTCTATAATGACTTAGGCAAGTTTTTAATTAAAATATTGATTATT |
| SEQ ID NO: 243 | TGTCTTAAAAAGAAAGAAAAAAAAAAAAAAGTCTCCCTACTATACCCTTAGATATATGCCC |
| | TTCCATAGATAAACATGGTTAACAATTTCTTATGTAAACTTCCAGAAACTTCCTATGCATAT |
| | TAAAGCATATCTATATTAAAATATGTATCTTGTCTATCCTTTAACAAAATAGAAATGGGGGC |
| | CGTTCTACATATACATACTGTTTTATACCCTGCTTTTTGGATTTACTGTCTTACAGCCATCTTT |
| | GTATGCTAGCACATGTAGATGTTCTTCATTCTTTTTAAAAACAATGTTGAG |
| SEQ ID NO: 244 | TGTTGCCCAGGCCAGTCTCGAACTCCTGAGCTCAAGCAATCCACCCGCCTCGGCCACCCAAA |
| | GTGTTGGGATTACACTGTGCCCGGCCGCCTCAGGAATTCTCTAAGTGGAGAATTAGTGGTGG |
| | GAATATTACACCTGATAGCTCAGAGGTCTCTACATTCAAATTTGTCCAAGGTTTATTACTGG |
| | GGTCCCTGGACATAATCCAAAGGGTTCACAGACTAGTTGGACTGGAGGAGGAATCACTATT |
| | TCCACTAACCTCTCTCTGAAATTTAGCAAGTTTTCTATTTTAAATATGGTAAC |
| SEQ ID NO: 245 | ATTAAAGTGGAGAATTTTCGTAATCAGAGCTTTATTGAAAAGTTTAGTGTGTATAGGCATTG |
| | TAGATGTGTCTGTGGAATTTATGGACTCAGTTAAAAATTGTCACCTGATAAAAATGAAGTTA |
| | ACTGTCTATTCAAATGATGAGATTTGCATCTACAGATATATTACTACCTAAAAGCCGAGGTT |
| | GCATATATTGCCGATGGACTCCTAGAATGAGTCAGGCATCTTTTAAATCTCAGTAAAATGAT |
| | TGTTATCTTCCAGATACTATCTAAAATATCACCAATATAAGATTCCTCATCA |
| SEQ ID NO: 246 | GTTTAGCTTGAGTGCATACTATGTAAAGAGGGTCTTACAAAATGTATACCGGAGCCACAGG |
| | AGAGCAAAACAATTATTTAAGTGATGACATCTGGTCAAGATGGCACAGGAGTTCAGGCTCC |
| | AAACACCTAGCAAAGGTAGTTATAATATAAAAAGGGTAACCAAATAAGTATAGTTGGGCTA |
| | TAGTCATTCTAGTTACCTAGAATGTAAGTTGAAATACAAAGGGGTAAGCAGGCTCAAGCTG |
| | CAGGCCTCTCCAGCAGGGGCAACCAGAAACTCAGGTTTTTTAGAATAAGGGGATAT |
| SEQ ID NO: 247 | ATTTTGGCCTCATCATCACAATTCAACTGAGACAGCCAGTAGGAAAAAAAAGTGTAGTCTG |
| | AAACATTGGAAGCAATGAGAAGTGTACAAAGAACATAGAATTTTTAAGGATATTAATATAA |
| | ATAAGACCTCCATTTAGAAATTAGAATGGCTTTATTAAAGGAAGCATAGAAAAGATTTCCA |
| | TTGAAGAATGTTAAAATACATATGTGTAGTATTTATATCATTGTCATATTTATAGTATATATG |
| | ACTTTTTTTTAAGATATGGAATATGTTTCTTTCCTTTTTTAAATCTTTGTAGTT |
| SEQ ID NO: 248 | GCATCTGAATACCCTGAAGCAGAACTGCTCCTTGGTCCGATCTCCCATACTTCTTTCTATTTA |
| | CTCAGCATATTAGTTCTTACATTTTTGTGGGGTGGACTATACTCTCACTTTAACCATAAGGTC |
| | TGGTGTACAGTAAATAAACACAAAAGCATCAACTAGATGAAGTAGAAAATGCCATAACTTA |
| | AGACTAAAGAATTATAGTACCACTGTAAGTGTAGGAAGCTTGCAAAAAGGAAAAGATAAA |
| | TTTATTCTTTGCACAGAGGCATGTATGTGCAAAGATAGTAAAACTCTATGGAC |
| SEQ ID NO: 249 | GGAGAATGTTTTAAGAGCCATAAAAAAGAACGAGATCATGTCCTTTGCAAGGACATGAATG |
| | GAGCAGGAGGCCATTAGCCTCAGTGAACTAACACAGGAACAGAAAACCAAACAACCACGT |
| | GTTCTCACTAATAAGTGGGAGCTAAATGATGAGAACACACAGACACATAGAGGGGAACAA |
| | CATATACCAGGGCCTTTCAGAAGGTGGAGGGTAGGAGGAGGGAGAGTCAGGAACAGTAAC |
| | TAATGGGTACTAGGTTTAATACCTAGTGATTACCTGGGTGATTGCTTAATACCTGAGTG |
| SEQ ID NO: 250 | ACTGTTTCTACAACAATTCACTTTTATGTGGGAAGTGGTGAGTAAGTCCTGATGACCCTGTG |
| | TTAAATCTTTAAGGCCAAATGTCTTCTTTGGTCCTGGCAGGAATAAACAAGTTTTTCAAAAA |
| | TGGTGTGCAAACACACACACACACATACATAATTACAACCAGCAAATCAGATTATAGAATT |
| | TTCTGAACAAATTCACCTCAAAATATATCTATTTATATTTATATATCAAAATTGGAATCACA |
| | AATAAATGGGGAAGGGATTGCAATTTCAATAAATTCAGTGCTAGGAAAACCTA |
| SEQ ID NO: 251 | GTGAGAGGGAACCAGTGGGAGGTAATTGAATCATGGGGCCAGGTCTTTCTCATGCTGTTCT |
| | CATGATAGCGAATAAGTCTCACGAGATCTGATGGTTTTAACAAGAGGAGTTCCCCTGCACA |
| | TACTCTCTCTTTTTGCCTGCTGTCATCCATGTAAGATGTGACTTGCTCTCCTTGCCTTCAGCC |
| | ATGATTGTGAGGCCTCCCCAGCCATGTGGAACTGTAAGTCCAATAGACGTCTTTCTTCAGTA |
| | AATTACCCAGTCTCAGGTATGTCTTTATCAGCAGCATGAAAATGGCCAAATAC |
| SEQ ID NO: 252 | TGTTGTCCTGTTGTTCTCACCAGGGACAGAAGCAGCTCAGAATGCTGGACTTCAAAGAGCTT |
| | TGTTCTTGCAGTCAGGGACTAGAAACCCTAAGCAGTGATCCGTTAGTGGACATAGGACCAA |
| | GGAAATGTCAGTAGTTGCCACACAGTGAATAACTTGACTGCTGTGTCTTAAGTTTTTAACTT |
| | TCATCCATGATGTTGGATATAGTTATAGTGCATTCATTTTCATTACTATATGGTATCTCATTC |
| | TATGAATATAACAGATAATTTATCTATTCTACTTTTTATAGAAACTATTTTG |
| SEQ ID NO: 253 | TATATGCTGCCCAACTATCTTCCGAAAAGCCTTCACTACATTATTATAATCTCACCAAAAGC |
| | ACATAGATGAGAATACCTATTGCCTATATTTATTAACACTGAATGTTATTGATCTATTGAAT |
| | AAAAAGTTATCTTAATATAAAATTTCAAAAAAAAAAAAAAAATTGAAGATAAGCCACAGA |
| | CTGAAAGAAAATATTTGCAAAAGACCTATCTGATAAAGGATTGTGATTCAAAATATACAAA |
| | GAACCCTTAAAGTTCAACAAGAAAATAAGCCACCCGATTGAAAAATAGGCCCAAA |
| SEQ ID NO: 254 | CTGGCCCAGGTCAAAAATGGAGATGATCAAATCTGCTGTTCTCATCAGTAATGGAATCACC |
| | CCTGTGCATAGCCACTCACTCTAGCCTGAGCAACATAGCAGGACCCTGTCTCAAAAAAAGA |
| | AAAAACCAAAAACGAAAACCCAAAACAAAAACAAAAAAATGATAAAGAGAAAACATTAA |
| | AAGCATCCAAAGGAGTGGGAAAAATACATTATATATACAGGAATGAAGAAAAAATGACAG |
| | CAGATTCAGAAATAATCCATGCCAGAAGACATTCAATCAGCAAAGAACTGAAGAATACT |
| SEQ ID NO: 255 | TAGCACATTTGTATCAGGCTAGTTGCCAGTGTTCAGGGTACGCCTCTCCAATCCAGTCCAGG |
| | TGGTCACAGTCTAGAGTTGTTAAATTATATGTGGTTCTGGCATGTTTGGTTTGACACCAGCC |
| | AAAGGTTTTATAAACCCTAGCATTATTGACATATAGTCTTAAAAATATTATTACCTCTTGGA |
| | CCTTAATGTCTTAATCTCATTGTTAGGTTAGTAAAATTAATGTGCTTCTGATAATCTGGTTTA |
| | AGTGTATTATGCCTTTTCTTGGGCTATATAATTTATGAAAACTTTATTTCT |
| SEQ ID NO: 256 | ACATTTGAGCAGAGATGTGAAGAATGGCATTTCAGGCAGAGGAAACATGAAGTGCAAAGA |
| | TCCTGAAGCTAGACATGATTAGTGAGTTAGAGCACAAATGAGGCCAGGCTAGGGGAGGAA |
| | GGCATGAGAAGGAAGCGGTAAGACATGCAGCAGTGACAAGGCAGTGGAGGGTGCAGCTCT |
| | AATAAGAACTAATAAGTTCTAATAAGTTCCACTCTGAGGGAGGTGAGAAGAGGGCTTTGAG |
| | TAGAGCTGTGATATAATCTTGCAAGAAAATTCCAATTGTTCCATTGAAAACACAGAAGC |
| SEQ ID NO: 257 | GCCAAGTTGTACTATGCCTTTGTTACGAGGCTACAGATCTCCTCGAGACTCTGGTTTATTTTC |
| | CTTTTGTCCTCTAATTCTTAAGTTTTTTTGTGATTATTCCGAGCTATTCTAGTTAAGCTAAGG |
| | GTAACCAGGATAAAAGTCTTGGTCTGCTTTGGCCAGATTATTAAGGTGGGAATGGTGCCCCC |
| | TGGTGTGCAGCTACACAGAGGACCTGGAGGAAGAAGGAGACCTGAGCTCCCTCCCTCCTCT |
| | GCTGCCTCCTCCATTTGGAAACAGACACAGGATGGTGATCAAAAGCACGTG |
| SEQ ID NO: 258 | TTGGGAGGCTGAGGAAGGAGAATCGCTTGAACCTGGGGGGTGGAGGTTGCAGTGAGCCAA |
| | GATTGCGCCATTGCACTCCAGCCTGGGTGACAGAGCGAGACTCCTTCTTGGAAAACAAAAA |
| | CAATAACTGTGTGAGGTTAGTAAATTATAAGAACCTAAAAGCAATATAAATGAGATAGTAC |
| | TTATCATATTGAAATGAATAAGAAAGGAGGAATTGAGGGAAAGCTGAATAGCTTTTTTTCCT |
| | TCTCTGGAGATAGATCTTTATAATATAACTTTATTATTTTTCAATAAGTGAGTTTC |
| SEQ ID NO: 259 | GTTTGTATTTCTGTGAGATCGATGGTGATATCCCCTTTATCATTTTTTATTGCATTTGATTCTT |
| | CTCTCTTTTCTTCTTTATTAGTCTTGCTAGTGGTCTATCAATTTTGTTGATCTTTTCAAAAAAC |
| | CAGCTCCTGGGTTCATTGATGTTTTGAAGGTTTTTTTGTGTCTCTATCTCCTTCAGTTCTGCTC |
| | TGATCTTAGTTATTTCTTGCCTTCTGCTAGCTTTTGAATGTGTTTGCTCTTGCTTCTGTAGTTC |
| | TTTTAATTGTGATGTTAGGGTGTCAATTTTAGATCTTTCCTGCT |
| SEQ ID NO: 260 | GGCCTCTACTCTTCTTGTAGTTTAAGCTGCCTCCATCTGGTTGTGATGGCCTCCCCTGATGTG |
| | TCAGCCTCTACATAAGGGGTGTACTTGTGAAGTTTCACACTAGACCTATGTGTCCAATATCC |
| | TCTCAGTCTCCAAGGGCTCTTCCATTTAACACCTTTTCTCCTTCAACCTTGCCATGAACACAT |
| | CTTCAAAATCAGGAGACACGGGCCAGCATTTCCCTCTTCTTTTCTATTATGAGGGAAGAGAG |
| | CTGAAAAGAAAAGCATGTCATACATATAGCAACAGAAGTCTCCAGATTCT |
| SEQ ID NO: 261 | TAGCACATTTGTATCAGGCTAGTTGCCAGTGTTCAGGGTACGCCTCTCCAATCCAGTCCAGG |
| | TGGTCACAGTCTAGAGTTGTTAAATTATATGTGGTTCTGGCATGTTTGGTTTGACACCAGCC |
| | AAAGGTTTTATAAACCCTAGCATTATTGACATATAGTCTTAAAAATATTATTACCTCTTGGA |
| | CCTTAATGTCTTAATCTCATTGTTAGGTTAGTAAAATTAATGTGCTTCTGATAATCTGGCTTA |
| | AGTGTATTATGCCTTTTCTTAGGCTATATAATTTATGAAAACTTTATTTCT |
| SEQ ID NO: 262 | ATGTTCACAGTAAACACACGGTTTTAGGAACTTGAAGAACGTCCATACATAGAACCCAGAC |
| | TCTTAACTCCAACACAGATCACTAAAGCTTTGGACTGAAAAGCAAGGAGTGCACAGGCCGA |
| | GGCAACTCTACCTGGGGGAGGAACACTTCCATGACCCCAAGGCCGGGTGAGCCTGTTGGTT |
| | CTGGCATTCATTGGTTAGTCACCTGATGTAGATGTTCTACTCCAGAGGTGGCAAGCGTGTGG |
| | GACATTTGTTAACACTCCCACCTCCAATACTTACAACAGACATCAATAATGGATC |
| SEQ ID NO: 263 | AATGAATATATAACAACCATAGAAAAATGTAGAAGTTTTCCATGTAGAAAACATATTCTAA |
| | TCACAGAAAAACTGTAGAAGACACTGTGATTGGTCAATTCAATATTTATCACTACTTTCCGT |
| | TCATTTCTAAGGACACTCCTGTGTTGGTTGGTTAGCTACATGTCCAGCTAAAAGTCTCAATTT |
| | TGTAGGCTTCCTTTCATCCAGTGGTGACCTTTTGACATAGTTCTGGCCAATGAGAATCTTAAT |
| | TTTCTTGGGTGATGCTCCTAGGAAAACTGCTGTTTTCAGATCAAAAGAAAC |
| SEQ ID NO: 264 | GAACATTTTTTCTAGTTTATTATATATTTAAATACATTATAGTTTTAAAAGCAAAATGTTATA |
| | ACATGCCCAGAGTGGTATAAGAATAAAATAACGTTTCAATAGAAATTTCCTCTCTTTGAACT |
| | TCAATTTATAGACAATACATAAAGCTAGAAATATGTTGAAACTTTCTAATAAAACTCAATGA |
| | ATTTTCTCTTTTAATCAGAATTATTAAACGAAACAATTCAAAATAACTAACATGTTATACAA |
| | ATATAATAAATTACACAGTGGATTTTTAAACTTGTACAAATAGTTTATAAC |
| SEQ ID NO: 265 | CATTCATATGTAAGTAGATATACAGTTGACCTTTGAACAATATGGGCTTGAATTGTGTGGGT |
| | CCACTTATACACAGATTTTTTTCCCACCTATGCTACCCCAGCACATCAAAACCAACCCTTCCT |
| | CTTCCTACTCCTCAGCCTACTCAGCGTGAAGACAAGGATGAAGACTTTTATGATGATCCATT |
| | GCCACTTAATGAATAGTAAATATATTTTCTCTTCCTTATGATTTTCTTAATTTTTTTTCTCTAG |
| | CTTACTTTATGGTAAGAATATAGTGTATAACACATATAACACAAAATAT |
| SEQ ID NO: 266 | TGCATACATACTTCCCATGAACTCTCTTACTGGAAGATATTCCCAGATGCCTGCTATATTCTG |
| | TGAACTCTAACCCTAGAAAAATAAAATATTTTCTTTTATCATAGTTTCATTTTCTTTATCAAG |
| | GTTAAGTTTCATTCATATTTTGCCATCATACTAAAATATTCCCAACTTAAGAATGTTTTTTCT |
| | ACACAGTTCTTGTCCTCCTCCCGCTCAATAGTATTATAGTTTTTTTACGAGATAAGGTACTTG |
| | ATTCCATTCATTTCTTAAATTTTCTTTTTTCAAGGAACCTCATGTGTA |
| SEQ ID NO: 267 | TTTGCAGAGTATTGAGGTGGCACAGGGCATCACACGGCAAGGAGAATGAATGTGCTAACAT |
| | GCTAGCTCAGATCCCTCTTCTTATATAGTTACCAGTCCCATTACCTGATAACCCATTAATCTG |
| | TGAATGGATTAATTCATTCATGAGGGCAGAGCCCTTATGATCCAAGTGCCGCTTAAACAGG |
| | CCACGTCTCTCAACACTATCACATTGGGAATTACATTTCAACATGAATTTTGGAAGGAGCAA |
| | ACATTCAAACCATACAAAATGGAAAAATAAATCAAAATAAAATAAACCTCCTT |
| SEQ ID NO: 268 | AGTTGCAGGCACATAATATGCACTAAAAAATGTCATTTCCCTTACTTTGGTCAAAATCCATT |
| | AACGCATTCTGTGTAAGTGTGGAGTCCTTATCATGGCATTCATGATTCACTGTAATCTCAAA |
| | ATCAGTTACCATTTTGGATTTACTTTCCTTATATGCCCCCTACACTCCAGTGAAATGAAACCG |
| | TATCCACCATGCAGCCATTGATGCAAGCATGTCAAGAGAGGGAGACTTACAAGAGTAAGCA |
| | GTAAGGATTCTGAGAAGCTGATATACCTTGGTCAAATTAAATTTTATCAGGA |
| SEQ ID NO: 269 | TCCGTCCTCATCCTTTTTTTGCTCTACTAACTTGTAACTCTTGATACATAAAGGCCATAACTT |
| | ACTGCAATTATTAGAGAATACCATCTCCCAGATACTTTCTTCTGCATGTCACAATAGATCAT |
| | CTCTTGAAGTATGCTGTTCCTCTAATTAATCTTCATATTGTTTTTTGTTTTTTTGTTCTTCAAT |
| | ACTTGCTTGGCTCCGTGGTTTTATTTTTTCTTAATACTCATATTTGAATAATGTGAGATAATG |
| | ACACTGGGGTAAATATTTTTCATAGGTAAGAGTCAAAAGATTGCCTTT |
| SEQ ID NO: 270 | CTCTCAAAGAGTGAGGGGTGTATACTCACTGCCATAAATCTAACAGTACTGGTCATAATTTC |
| | AAAGGTTTCATATATTTATCTGAGATGAGTACATAGCTCAACCTCTTTGTCCTTACTCAGCTT |
| | TTTTCTTCAATTTATGTGGTACATTTCAGAAAACACTGTACACATGACAAGATATCCTACCC |
| | CATATGTCTAAAATTACATGTTAAAATAAATAAAACAATTGCAGTGCTTTGAAAACTTGATA |
| | ATCTGGTGCGTGTCCTTCCAAATTTGATTTACCTAAACTAGACTACATGCA |
| SEQ ID NO: 271 | ACTGATACATCTTTTCTCTTACATCACAAAATATTGTGTCTCCCCTGTCATCTCTCAGATGTA |
| | TCCTTCCACTTGTTCTCCATCCTCAGAGATGCTGTTCCATCAATCATTCTGTCTCTCTTATATA |
| | TCGTGTGTATGTATGTATATATATGTGTGTGTGTCTGTGTGTGTATGTATATATGTGTGTGTA |
| | TATATATACACACACATATATTTTATATATATATCCTCAACCTTTCCTTCATTCTGCATGTAT |
| | CTTTAAACCTTTCCTTCATTCCATTTAGTATAAAAATTTGTTTAAAG |
| SEQ ID NO: 272 | AAAGTGGGTGGGGTTTGTGTTGATGAAGTCACAGACCTATCTTCACCGCAAACCTTCCCAGG |
| | GTTCATCCCATAGCTAAGACCAAAGTGATGCATGTCTACCCAGGGGCAGGCAGAGGACATT |
| | TGCCCTTTTCTCATTCACCTCTCCCCAAGAGGTGACTTAGGAACTTCCCTAAGAAGTTTCCTA |
| | GTCAAAACTTCTTTCTTGGTGACGACTGGTCACTGTTTACAGCCCCTTGTTTCTCTCCTCTAT |
| | TGGAGTGGTTAGTCAGTCGCCTCAACCCTACTAATAAAAATAAACCAGGTG |
| SEQ ID NO: 273 | GCTATCCATTTTGGGGGCGATATCTCTTGCCTGTTCTATCATTCATTACATGCACTCAGTTGA |
| | AACAACAATTTTAGGCTTCTGGAGCCCAGGTCTCCTCTATCAGGACTAATTCTGAGTGCCAA |
| | GATCAATGACCAACATCAGAGGTAGTGGAGTGGTTAGGTGCATGGCCTTTCTGTCTGGCAG |
| | ATTGAGTTTATGTCTTGGTTATGTCATTTCCTAGCTGGGTGACCTTGGTAAAGTCACTTAACC |
| | TCTCTGAGTCTTCAATCACTTGTGAAATGATGATAATACTACTGGCTACCA |
| SEQ ID NO: 274 | AATAACTTATGGGAAAAGCTTTTATACTTGTCACTCACTTTTTAAAATATCCCGAGACAGTT |
| | CACTGTTGCAGACATTGAAATTGGCCATTTGTAAGATAAAAGGTATGTTTATAAAATCTCTT |
| | TATATAATATATGCTATCTATGACATGCAAAAAAGAAAAGTCTGGGTGCTGAGGTGCTGAA |
| | TTTTTCATTAGAAAAACATTTGTATAAACTACTATTATATAAATATAAGCATATTTATTACAG |
| | CAAACATTTTAATAGCAAACAAAACAATTGATCTTAAAAATATATGCAATAT |
| SEQ ID NO: 275 | GCTATTTGGGAGGCTGAGGCACAAGAATCGCTTGAACCCAGAGAGTAGAGGTTGCAGTGAG |
| | CCAAGGTCACACCACTGCACTCCAGCGTGGGTGACAGAGTGAGATTCTGTCTGAAAAAAAA |
| | AAAAAAAAAGATTTAGTAGTATATAAAAAAAAATATTTAACTTCCCTATTAAAATAAAAAT |
| | GCTTTCAAGATTGGGTAACGAAGTAAAGTTCATCTTGGGCACTGTGACTAAGAAAGGATTG |
| | GAAAAAAAGAAGGTCATCAAAGGTATATCAGGCAAATGGGAACAAAAGAAAGCAGT |
| SEQ ID NO: 276 | GCTGCAGCCCTCACCAGACAGCCAAACCTGCCAGCGCCTTGATCTTGGACTTTCCAGCCCCG |
| | ACAACTGTGAGGAAATACATTTCTGTTCTTCTTAAATTACCCAGTCTTGGGTATTTTATTATA |
| | GCAGCACAAATGGGCCACAATCTGCCATAGTACCTGTCATGTAGGAAGTATTCTGAAACTA |
| | TTTATGGAGTGGGTTTCATTAAAAGTTACTCTAATCTTTGAAATAGAAAAATATCTTACTTTT |
| | ATGATCAATGTTTTGTTACAAACTGTCTCAATTATAGAAACAGGCTTTTGC |
| SEQ ID NO: 277 | TGCTTTCTTCTACTATTTTGGGGTTTTGATTTCTTACATTTGGAATTTATCCAGGTGTAAGGT |
| | GTGAGGCACGGATCCAACTTTATCTTTTCAAGATGGCTTCTTCTGTGTTGATTCAACACCAC |
| | CGATCAAATAATCCACTTTCTCTTTACTCAATTGGGATGGCATCTATATAATGTATTACATTC |
| | CCAGATGTGTTTCAGTTTATTTCTGGATTCTTTATTTTGTTCCATAGACGTGTCTGTCTGTTCT |
| | GAGGCAGTACCATACTGTTTAGTTACTGCCATTGTAGAATATAGAGTT |
| SEQ ID NO: 278 | GTAAATATGAGAGTATGTGACAGAGACACTCTAGAAATAAGCAAGTCAAGGTTTAGTGGAA |
| | GTCTTGCTCCATTTGGGATACTTATTATCCTAAGTCAACAACTGATGGTGCCCTGGAGTCTCT |
| | TTTTATCTAAGACTTCTTAAATAGATCCTTCTCTGCTACTTAGTATAAGAAAGGAAGTTAAA |
| | TATGTCCAATAAAAGTATACTGTGGTTTTGGCATTTATTTAAATGTATAAAAACATGTACAC |
| | ATTTATTTAAATGTATAAAAACATGTACACATTTATTTAAATGTATAAAAAC |
| SEQ ID NO: 279 | TTTGATCTTCTGTCTTGATACTAAAACTTTCTGCATATTCGTATGTTCACTGGAGTAGGATTT |
| | TAAATTTCCCTTCAGAACTTTTTCTTTGCATTCACAACCTGGCTAACTGGCATAAGAGGCCT |
| | AGCTTTAGGCCTATCTTGGCTTTCAACATGCCTTTCTCACTAAGCTTAATCATTTCTAGTTTT |
| | TGATTTGAAGTAAGAGACATGTGACTCTTTTCAGTTGAACACTTAGAGGGCATTGTAGCATT |
| | ATTGACCTAATTTTAATAGTGTCCCAAGAAAATGGGAGACAGATGGAGGA |
| SEQ ID NO: 280 | GAATTCTGACTCTGGTTAGTTATGTGGCCGTGGCCCAGTATGTGATTGCATATTGATGTTGA |
| | ATGAGACACAAATTTAACCCTCAAGGAGCTTATACTTAAACAGAAAAGGCAGAGTCAGGGG |
| | TTGGAGAGATAGTTTGGCTTCAAGGAGGAAGCACTATTTAAGCAAAAGCTTAAAGAACAGA |
| | TTAAGTTTTCAGGCATAGAAGGAGGTGGAGTGACATAACCAGAAGCCCCAGCTGAGTCATA |
| | CCTGGAAGACAGACAAAAGTTTATATCAGATGGATTGAAGGGTATGTAGGAGAAG |
| SEQ ID NO: 281 | GCGGCTCTAGCGCGCGGGAGCTGGGCGAGGCTCCGGGACGACCTCACCAATGGAGACTGCA |
| | GTATTTAGCATGCCCCACCCATCTGCAAGGCATTCTGGATAGTGTCAAAACAGCCGGAAAT |
| | CAAGTCCGTTTATCTCAAACTTTAGCATTTTGGGAATAAATGATATTTGCTATGCTGGTTAA |
| | ATTAGATTTTAGTTAAATTTCCTGCTGAAGCTCTAGTACGATAAGCAACTTGACCTAAGTGT |
| | AAAGTTGAGACTTCCTTCAGGTTTATATAGCTTGTGCGCCGCTTGGGTACCTCG |
| SEQ ID NO: 282 | ACTACAGTCAACTCTTTTGAGATGGAGTCCAGTTTCATGCCTAGACTGAAATTCAAGATTCA |
| | TAATGAAGAATTCACTTTTATCAAGCATTGACAGGAGTTAGACACATCTCAAACCATATTTT |
| | AAAATACAGCCAGATTACAATGTTACCTGATTCTGAGACTGCCTGTAACTTGATTCATAATA |
| | GTGACAGACAGCACTTGGACTAGGACACTAGAGCTGCTGAGAAGGAAAATTGCACCCAGCT |
| | TCTTTGCTTCCTGCAAAGATGCACCAGGATCAGAGAAGAAAAGACCAAGTTCT |
| SEQ ID NO: 283 | TTCTGTTCAGGTTTTTGACATCATTTATTGAAAACACTTTTCTGACTCTATGGAATTGCCTTA |
| | AAATCTTTGTTGAAAATCAATTGACTAATAAGCATGAGTCTATTTCTTGACCTTTTTTCCTGT |
| | TGCATTGGCCTGTTATACATCTCTATGTTAATAAATACCACACTAACTTTATTGTTATAATAT |
| | TATACTAAGTCTTGCAGCCAGGTAATAGCATAAGTCCTCCAAGGTTTTCTTCTTTTTTGAGAT |
| | TGTTTTGGCTATTCTAGATCTCTTGCAATTCTATATAAATTTTAAATC |
| SEQ ID NO: 284 | AAAAGTGCTAATATAAAATATTCTCTCACAAAAATAAAAACTAACTCAAATGGCTTTTAGTT |
| | CTTGTGAGCTGAGATAACCTTCAGTAAGTAAAGCTAGTTTTTAAAAATGTTGATAAAATAAA |
| | AACAGAAATGTCTTCAAAATTGTCAGCATTTACATTATAAGTGTGCATTTTTAAACCTAGAT |
| | TTAATATCAAATGAGCTTGTTATCTCCTAGATATATGAGATCATAAAGCTATAAATCCTGTC |
| | CTTGGCTAGTTTTAAGGAGCAATTCAAGCATAGTTATTATGAATGAGTCATT |
| SEQ ID NO: 285 | CATTTGGGAGCTGATTAGGTCATAAGGGCGGAGTCCTCATAAATGGAATTACTGTCCTTAGA |
| | AAACAGACCCCAGAGAAAGCTCTGTTTTAGTCTGTGAGGACACAAAGAGAAGATAGCTATG |
| | AATCAGGAAGAGGGCCCTCATCAGACACTGAATTGCAGGCACCTTCCTCTTGGACTCTCTAG |
| | CCGCTAGATTGTGAAAATAATCAGACGTCTTCCCTAATCACCTTAATGTAAACAGGTCCAAT |
| | TCTAATTGGTCAATATTCTCAGCACTTTATTGCTTTCAAAATAGTATAAACGA |
| SEQ ID NO: 286 | AAAGTGTAGAAAGTACAATCACCCTGTTTGGCTGAATCAATCATCATTGTAGACCATGGAC |
| | AGTGCAAACCACAAACAATAATTTCCTGCTGCTAGAACTTGGCTGATGACCGTAACACCTTC |
| | CAGTGAAAAATAATCATTCACCATTACAATAATCAAATAATCATTTTTAACACCACATTCTG |
| | AGAATTGTACCCTAACAAGTTCTTTGAGATAATCATTCTAATATTTACTTCAATTTATTTATA |
| | ATTTAATAAACAAAATTTATTCTAGTTAATTGCAACTGCCAAAACCTGGCCT |
| SEQ ID NO: 287 | TCTCAACCTAAAACTTGGAAGAACCTGAAGTAAATGGTCAAGTCAATGGATTTCTTTTTAAT |
| | CCTACAAAAGTGAACTCTTGCACTCAGTAGAAGCCCAATAAATATTTGTTGTTGAAATTACA |
| | ATATACTATACCGAAAGAAGTAACCTAACATTGTAACAGAGAACAATAGGATTTTGTTATC |
| | TAGGTAAGGAAGTTAAACATAACAGCAGGTAGCACAAAGGAATCAGCTTAGCAATAATAA |
| | ATTTCAGATTATGAGGAATAGAAAACATTACAATTACATATAGAAAATACAAAGC |
| SEQ ID NO: 288 | CCCCGGTGTGAGAGTCTTAGGTTTAGGGAAAAATATTACAAGTTTATTTAGTTCTCTTATTTA |
| | ATTACTTAAAAAAAAAGATTACATGCTAAAATATGTTAAATAAAAATATAAATATAAACCA |
| | ATTGATTTATCTTTCCATAATCTTAAAACATAACTAGAGTAACAAAATCAAGAAGTGCAAA |
| | AAGATAAGATTGATAAATGTTTAGATACTAAAACAACCACTTCAAGTGTTATAAAAGAGTT |
| | ATGTAGAGAATTACTGAATGTAGAGGAGAGCATTTACTTCAAAAATGTTGGAGC |
| SEQ ID NO: 289 | TGAGTATCTGGCAGATATTTTCTCAAAAAGGAATAAAATGAGTCTGTCACTTCAGGTAAAA |
| | CGATTTTCCTGAAGTATCTGTTGCCAAATGACTAAATTTGAGCTTTTAAGTAAAAACTAGTA |
| | TTTTGAAAAACTTCTATCTATTACCAATGAGCTTTGACAGCCTCCCAATCCTCGTGGATAAA |
| | GATCCTTTCCGAGTATAAGACAATCAATGGATTCTTTTTGTAACAAGTACAAAAAAAATTCA |
| | CTGATATGATTTCAGATTCCACATTGAAACTAGACTTTAAAAAAACTACCACT |
| SEQ ID NO: 290 | GGGAGGAGGGTATCATGAGGCGTGTCCCATATGGCCTGAACTGGTTTTTCAGGTTTCTTTGG |
| | GTCCTCTTGGCCAAGAGGAGGGTCTGTTCAGTCAGTTGAGTGGCTTGGAATTCTATTTTTTT |
| | GGTTTATAAGACCATGGTAATTTCTGTATTTAGTCAGCATTTAATCACTAAAACTTAATCTTA |
| | GTAGACATTTTTTAAAATGCTGAACTAAAGTGAGATTTTCTCTTCCTTAAAAATCTGGGATG |
| | CTCGTTCATTCTCTGATCAGTTCAGATGCAGTGTATGAAGAACCATAAAGC |
| SEQ ID NO: 291 | ACTTGACATTGTCACTTTCGGTGTTATCTTCATTCTGTTATAAGAGAAAACTTTCGCTAATAG |
| | TTAAATGCTGCCAGGTGTAAATTAGTGTCATCAGTTGCTTCAAACAGAAAATAAGCTCAGTT |
| | AATTTCAAAATTAGTTCAAAGATTTTTTATCTGATTTATGAAATAAAAGAACTATTTTGGAA |
| | ATTCATTCATTCAAAGGTACAATATAAGATATTTTATGAAATTTACTTAAAAAGTTCAAGAA |
| | TAAACATCATGGTAGAACAATATTTTTCCTAAATTAAGAAAATGGTGAAGC |
| SEQ ID NO: 292 | TTCATTGTCTCGAATTCTTACCAAATGATTTTGGAGAGAATTGAATTGATAGAATTGATTATT |
| | AGAAAAATGAGTTGAAAACAAACTATTTAGAACCTACCTCTGTTTTTTATGCATAAAAAAC |
| | AGTATCAGAATATAATGAATATAAGGCTAATGAAGAATGCATGTGTAAGACTTGCTTCAGC |
| | AAAGCTAAGGAATGTTAAGACAAAATTTCTCCTACCAAGTCTGAGGAGCAAGAGATTAAAG |
| | GATCTGAGGAGCAAGAGATTAAAGGATCTGAGGTGCAAGAGATTAAAGGAGAGA |
| SEQ ID NO: 293 | GGATTTGGGATTCACCAGGGCACAAGGCGACGTGCCTCTCTCCTCACGGACCCTAAGCGCT |
| | GGTGGACAATGCAGATGTCAACCGCAAATGATGAGGCACTTGCAGGTAGCGTTCAGCACCA |
| | AGAAGGAAATGAAGGGTGCGTGTCGCTGACTGTGACTCTGGTGGGGGCAACTTAAATAGGG |
| | CCATGGGGGAGGGCCTCTCTGAGGAGGTGACATTTGAGCTGAGGCCTCACCAGTCAGAAGG |
| | AAAAAGCTGTAAAGGGTCTGGGATAAACTCAACCTGCAGACTTTTAAAATAATATA |
| SEQ ID NO: 294 | GAAATCTGTAGGGCAGGCAGTGAAGATGGGAGGCTGGAAGCCTCAAGCATAAGTTGAAGC |
| | TGCTGTCACAGGCAGAATTTCTTTTATCGTCAGGAAACTTCAGCTCTGCTCTTCAGGCCTTTC |
| | AACTGATTCAGTGAGGCCCACCCAGATTATTTAGGATAATTTCCCTTACTTAAAGTCAACTG |
| | ATTTTGAACTTTAATCGCATCTACAGAATACCTTCAAAGTGACACTTGGATTGATGTTTGAT |
| | TAAATAACTGGGAATTGTAGCCTAGTCATGTTGACATATGGAAGGATCACCAT |
| SEQ ID NO: 295 | CCTATTTAATCAGAAACTCTGGACTTGCAGTCCAGCAGTCTGATTTAACAAGCCTTTTGGGT |
| | TCTCCTAATCCCACTGATGTTTGCAAATGACTGACCTAGGTCTTTGGTCCATTTTAGGTTTGA |
| | TTTGTTGGCAAGACTACTTCATAGTAATGGTGTATTCTTTTAAAAGGCATATAGTATCTGATT |
| | ATCTTTTATTTTATAACCAGCCATGCTCAGCACTTAGATCCATGAGGGTATAGTAGAAATTG |
| | CAAAATGGAGATAGCGTATTGTCCCTTCTTCACTTATTACCTGAAACACA |
| SEQ ID NO: 296 | CCAAAGCAGAGTTCACCCCCGACCATTTCAGAAGGTATTTCTGAAAAGGAGCAGATAGAAC |
| | GTAATTCGGAGTCACTCACCCAGGTGAGAGGGTTGGCCTCAGGTCTAAAGGCAGGAAGCGC |
| | TTCTCTGTTTATAAGACCTAATTTTACAAATCCATTCAAAGCTTTCATATATCCCTGAAACAA |
| | GAATTAATAAGCAGTATATAAAATTTTTCTCTTCCAATGAAAAAACTTAAAATATTTTTGAA |
| | AAGAGGGATATATCTTTGTAACTAATTTAGAAAACTGTTTATATTGATGATAT |
| SEQ ID NO: 297 | TCAGGCCGGCCATGAGACCGGGAAACCCAAAGCGCGTGAGGACGCGAGCAAACTAGGCCG |
| | GCGCACGCGAGCCGAAACGCTGGCTTTGGTAGGACAACCAAGCTCACACGCCGAGAGATTC |
| | CGGAAGTGCTCGTTGCCCAAAAGAGACCGGAGGAAAACATGGTCGGGAGGGGGATAAAAG |
| | GTTCCGGAATGAAAAAATAATGGCGCCCCGGGGCGTAGCTTCTGGAGCGGGGCTCCGCCCC |
| | CTGCCCTAGCATCGCCCTCGCCACTTTTGTGTAAACTTGGCATCAATAATTTCTACCC |
| SEQ ID NO: 298 | TAAATCTAAAGGTTTGATGAATTCAATTTAAACCTTTTTGGCTGCAGTATGTCCTAGGTAAT |
| | CTTGGGAACTGTACATTGCCTCACATCAGAAGGCAAGATATTTGGTTAAACTACCAGAACTT |
| | GTAAGATTGATCAGAGTTTGGAAAAATTTATTTTGGAGAGCTTTTTTATTGTTTTGTATTTGA |
| | CCATTATTTCCATAATTGTATTTGAGAATTTTTGGTAATGACTTGTCTTTGCTGTGTTTGTAA |
| | CCTGCTTGAATTTTAAAGGATGATGCAGTAAAGAGTTCAGATAACTCTAC |
| SEQ ID NO: 299 | AGTAGCTGGGATTACAGGTGCCTGCCACCACACCGGGCTAATTTTTTATATTTTTGGTAGAG |
| | AAGGGGTTTCACCATGTTGGCCAGGCTGATCTCGAACTCCTGACCTCAAGTGATCCGCTCAC |
| | CTTGGCCTCCCAAAGTGCTGACATTACAGGCTGACACCATGCCCCGGCCCCTTCTTTTGAAT |
| | TTTGAGTGACCACAGGCATTGATGATAGAATGTGAATACATTTGATTTGTATTCATATGGAG |
| | GGAAAGTGCCTCTGACCTGTCCCCATGGAAGGAAAATAAGATTGGCCTGGAG |
| SEQ ID NO: 300 | TCATAGATCTAGGAAGCTCAGAGAACACCAAACAGTATAAATATAAAAATATCTACATGTA |
| | GCCATATCGTATTCAAACTGCAGAAAATCAAAGACAGAGAAAAGACTTCAGATAGACTCAT |
| | CATCATCATCCTTCAACAGTCACTGCATGTGATGTTGGATGATTTCCTCTTGGGGTTGGTTGG |
| | TCAACCATAATGGATATCATAGCCACAATCTTACCATGCCTTCTTCATCTCTGACTCTAAGG |
| | AGTCACCTTCCTTTCCACTGTTGCCTGGTTCTGTAGTCTGTAACAGATTCATA |
| SEQ ID NO: 301 | AGAGTGAGGAATATAAGGAAATACCAGTAAAGAAAACTTAGGATTCTAAGTGACAGCAGG |
| | TATCTTAGTAACTGGTTGGTGATCACCTAATATAAGATAACCACAGGTAAGATGGGTAAGA |
| | CCTATTTTAGAAACCCATAAATTTCAGGGGCAGGCAGGTAGACATACTGTCCTGGTACACAT |
| | TTGGAGGAAGGATTATTTTTCTGAGCAAAGAAAACGATCTGTTCCCCTACTCATGTGGGCTT |
| | GTATTCATGCATATATTTACTTGTTAATCCATTCCTTCAATAAATGGGAATTCAA |
| SEQ ID NO: 302 | CTGTTTATGAGTTAAACTAACATTGTAGCTGGTTCTCTGAAGAAAAAAATACATAATTTAAA |
| | ATACAAAAACCACTTCTTAACAAAAAAGTGACAATGAAATATATAGAGAACAAGAATATTA |
| | ACAAAAATAAAACGAACATTAGAGAACACATAAACTTCATACAAATAAATCTCAAAACCTG |
| | GATGTAAAGAATAATTTTCTAAAACTCATCCAGAAGAGACAGAACATTTATCTCTATCTACC |
| | AGCACTACTAATTTTTAAACTGTACTGAAACTACAAGCTAAAGTAGGTAGAAAA |
| SEQ ID NO: 303 | AATGGAAATTATCTCAATGTAATAAAGGCCATATATGAAAAGCCCACAGCTATCCTACTTA |
| | ATTGTGAAAAACTGAAAGGTTTCCCACTGAGATCAGGCACAAGGCAGAGATGTCCACTCTT |
| | ACTTACTTTTATTCAACATAGTACTAGAAGTCCTACCTAAAGCAATTAGGCAAGAAAAAGA |
| | AATAAAACGCATTCAAATCGGAAAGGAAGAGGTAAAACTGTCCATTTGCATAAGACATAAT |
| | CTTAGATATAAAAAACCATTTAACAGTTTTTTAAAACTGTTAGAACCAATAAATTT |
| SEQ ID NO: 304 | ATAAAACTGAAAAGCTATTAAGTGGCAAAATAGGTAAAATTTCTCTGAAATGAAAAAAGTG |
| | AAATGGCAAAAATATAGCAGGCATCCAAGCAGAACATTCATGTCATCAAAAGGAGAAAAA |
| | TACCAAGGTGCCCTTGGATTTCTTCCGGCAATATTCAGTGCTTGGACTTAATAGTGAAGTGT |
| | GTTCACAAATCTGAGGCAAGGAAGGCATGAACCATTAATAACACTCCCAGAAAAGCATAAA |
| | GGCAACAGTCAGACACCCGAAGCATGAATGATTTCAAGGATTAAAAAACCAACTAG |
| SEQ ID NO: 305 | TGGCTCACGCCTGTAATCCCAGCACTTCAGGAGGCCAAGGAAGGTGGATCACAAGGTCAGG |
| | AGTTTGAGACCAGCCTGACCAACATGGTAAAACCCGTTTCTACTAAAAATACAAAAATTAG |
| | CCAGGTGTGGTGGCACATGCCTGTAATCCCAGCTACTCAGGAGGCTGAGGCAGGAGAATCA |
| | CTTGAACCCAGGAGGCGGAGGTTGCAGTGAGCTGAGATTGCATCATTCATTGCACTCCAGC |
| | CTGGGCAACAGAGCAAGACTCTGCCTAAAAAAAAAAAAAAAAAGACAGTACATGGG |
| SEQ ID NO: 306 | AAACTTTTCCAAATTTGATTAAAAGGATAACATATTCTAAAGGTATTCAATATTTTTACTTAT |
| | CTCTGAAAAACTTAATCACATAAAAGCATACATTTTACACATACAGCTCTCTCCATCTTCCA |
| | CAATAGATTAAGACATAAAACATAACCAGTATTTTTGAAAAGCCCCCTTAACTGGCATGCTT |
| | CTTACTGAAATTATCATAAAAGGTTCGTATGAGAAAGGATTCCAGAATATCCCTTAATTGTG |
| | TTGTAGCTTATGCATTTCTATTTATTTTATACATTATTTAATTCATGTGAG |
| SEQ ID NO: 307 | AGGTCAGGAGATGGAGACCATCCTGGCTAACATGGTGAAACCCTGTCTCTACTAAAAATAC |
| | AAAAAATTAGCTGGGAGTGGTGGCGGGCGCCTGTAGTCCCAGCTACTCGGGAGGCTGAGGC |
| | AGGAGAATGGTGTGAACCCGGGAGGCGGAGCTTGCAGTGAGCCGAGATCGTGACACTGCA |
| | CTCCAGCCTGGGCAACAGAGTGAGACTCCATTTCAAAAAAAAAAAAAATCCATATATGTGA |
| | CTGTAATAGTTCTTTGTTAATATAGCCATAAGAAATTATTTACATTAAAACAATCAG |
| SEQ ID NO: 308 | GATGTAATACTGCATCTTTATGTTTGTTTCCATTTCTTTTGACTGCGAATGGTGCCATGTACA |
| | GTCCGTGTTTGTGTGTGTGTAAGTTTCGTTAACTTTTAACTTTATATGAAAGATTTGTGTATA |
| | TTTTATGGTAGTCAATGATAAAATAGACTAGTGTCTACATACATTTTATGCATTCATGACAT |
| | ACCTTTTTCTTAATTTTGATATTTCTAGGCTGCACAGTTCATCTTTGAATGTTTTCAAATTATT |
| | GCAAATCTACAAAAAGTTTTTCAATATATGTATTGAAAAATATCCATG |
| SEQ ID NO: 309 | TCCCAACTAAAAAATGGGCAAACTATTTCAATAGACCTTACTCCAAAGAAAATATGCAGAT |
| | GATCGGGGAGCATATAAAATGATGCTCAAAATCATTAATCATTAGAGAAATGAAAATCAAA |
| | TTCACAATGAGATACCACTTTACACTCACTAGGATGGCTAACATCAAAAAACTGGAAGATA |
| | ACAAGTGTTGGTGAGGATGCAGAGGAATTAGAACCCTTGGGCATTGCTGTTAAGAATGTAA |
| | AATGGTATTCCTGCTCTGGAAATTGATATGATAGTTCTTTAAAAAATTAAATAGAG |
| SEQ ID NO: 310 | CCTTCCTTCCTTCCTTCCCTCCCTCCCTCTCTCTCTCTCTCTCTCTTTTTTTGGTGCATCCATAC |
| | TTTCTGGCACTATAAGATGCTTAGGGCTTATCTTGTATATTTCCAGCCCCAGTTATAGAATCA |
| | GCCATTTCTTCAAGGATCCCCTGTCATTTTACTGGAGAAAGGACTTAGAATCCAAGATCTGG |
| | GTGCTAGATGTGCTTGAGGTGTTGTTTCCTCCAGATCTTCTCAACTGACAGAGCAGTGAAAT |
| | ATATGTGTGTAGGGAGGATGGGGAGAACAATTTTTTTAACACATATGA |
| SEQ ID NO: 311 | TAAACTCAAACATCGCCTAGAACTTGAAAGAGCTTTCTAAATAATTAAATATAAAATTCAAT |
| | TCAAATTATATTCAGAGTATTTTCCATGTTAAAATTTTTGTTTTTTTTCTAGTTTATGGAATTG |
| | AATAATCCAAGTAAAATTTTCTGTAATCTCTTAGCTACTATAACATTAGATTAATTGAAACA |
| | GTCCATTCCCCAACCATTTGGGATAAACTGAAAACTGCCATAACATGATACGCCTTACTATT |
| | AGTACATATTCAAGAGACTTAAAGCACACAAGATTTGATCATGTTATAAA |
| SEQ ID NO: 312 | AGATTTTCAGATCAATCATATTTTGTTCAATGAAATTTGCATATAGACAAATAAACTCTCCC |
| | AGAAGAGTTTTACTTATCAAAACCAACAAACTCTCCTAGAAGTTTATTTGTTCAAACTGCAT |
| | TTTTACAAGGACACTTCCTAAAAATTGTAGTGTGGTCCAAGAGGTTATGCATATGTAGGGGT |
| | AGGAAAAACCGTGACAGTCAAAGGTAAGGTTCCAAAATACAAGTCTCCTCTAAAATGTCAA |
| | TAAACACACAATGAATAACACAGAGGGATGACAAGCCATCTGAAACTACAAAT |
| SEQ ID NO: 313 | GGGGCTCCCATTGGCCAAATATGGAAAAATTTGAGAATGAAAATAAATATTGATAGAAACT |
| | ATTACCAGTTGAACAAAATAAACTATGAGTCCATAATGATATAAATAAATGGAATTTGATG |
| | AGTAGCAATATATTTACCTAATTTCAAATACTGCCTTAAGAAATGCTAACAATTTTAATGGG |
| | AAACATTAGAGAAATCCAAATTGTGAGACATTCTAGAAAATAACCATCCTGAAATCTTCCA |
| | GAATGTCAAGAAAATGTTCTCATCACAATTGAAGGAAAATAAAGAGAAATAACTA |
| SEQ ID NO: 314 | GCTGAAGTCTTTTTATGGGCACAGGATAGGGGCGGGGCAGGCCAAAAAGGCAACATTTGGG |
| | CTGAAAAATAGGGTCAGCTGTTTTCACTTAGGACAAACTTAAGGGTGGGGCTTAGCCAGGA |
| | TCCCAGCCTTTCTGTCAGTTTGACCTTAAAACCTGCAGCACATTATATAGCCTTACTAGGGG |
| | GGATGTGTGTGTGTACACACACTTTTTTTTCCCACATACTATTATTGAACATTTTAAAGATTT |
| | GTAGTTGGCAGACTTTTGACACTTTGAGCAAGTTAATAAGAAAGCATGTTTTA |
| SEQ ID NO: 315 | GGTCAGGAGTTTGAGACCAGCCTGGCCAACATGGTGAAACCCCATCTCTACTAAAATACGA |
| | AAATTAGTCAGGCGTGGTGGCAGGTGCCTGTAATCCTAGCTACTCGGGAAGCTTATGCATG |
| | AGAATCGCTTGAATGCAGGAGGCAGAGGTTGCAGTGAGCTGAGATCACACCACTGCACTCC |
| | AGCCTGGGTGACAGAGTGAGACTGTCTCAAACAAACAAACAAACAAACAAAATATATATAT |
| | ATATGAACATTACATATATATATTTTTCTTTATGGGCTATGAAAGTATCATACTGT |
| SEQ ID NO: 316 | TAGATTGTGCAGTAAGATAATTGTCCATCTCCTCTCTGATTTACCTACTATAAGAAACACCC |
| | TTACACTCAACAGCCCATGGTACGCTAAACCCTCATGAATAAAACTAGGGCCTGAATACCT |
| | ATGGTTTACTCCAACTATAGGGGTGGGAGTAACATGAAGGGAAACCTTCCATAGCTGTCCA |
| | TGGATGAAACCAGCTGAAAAGCTCCCATAGAGGCCAGACAGGGAATTTCACAACTATAATG |
| | TGTTCTTCAGAAACATGCATGAGGAATTCTCTTGTAGTATAATTTAAATTATCTC |
| SEQ ID NO: 317 | CCAGTCTACTCTCACTAGTAGTTCACTTAGGTTATTATTTTCCCACATCCTTGCAAATACTTG |
| | GCCTTATCTGATTTTCTAATGCTTGTTTATCTCATGGATATCTAGTTGTAGTTTTTGAATTTGT |
| | TTAAATCACTCTTAGCTGATTTTTAATACATTTGAGCATTCTTCATATATCCTTTAGCCATTT |
| | AGGTTTCCACTTCTGCAAATTGCCTATATACATTCTTTGTCTATTTTTCAATTAGTTTTCCTGA |
| | CTCTTTATTATCGAATGATTTGCATATGTTTTCTTGTACATCCTAA |
| SEQ ID NO: 318 | AACAAACAAACAAAAAAACAACCTGTGGTTGGTCAAACAAATTTTGAACACCCGAGTCCTT |
| | ATGGGGTTAAAGTTTCTTTACCCTGGCTTTTCAATGAAGGAAAAATATATACTGAATATCTA |
| | GTCATATGCCAAAGCATGCTCCCTATGTTTTTCCATTTAATCTTAAAAAAAGAAAGACACTG |
| | ACAATAATAAGTGTCCTAATTTCATAAATGAAGAAACTGAGAGTGAGCATAAATGTAATTT |
| | AAATTGGAGATTCTAAGGAATCTTCGTAGCACATTATTATAGATTTACTTCCGT |
| SEQ ID NO: 319 | AGGCAGAGGTTGCGGTGCGCCGAGATCGCGCCACTGCACTCCAGCCTAGGCGACAGAGGA |
| | AGACTCTATCTGAAAAAGAAAAGAGAAAAAAAAAAGGCATCATTTTCTAGGTACCGTGACA |
| | GGGAATAGGTTGGGAAGCAATGACTTCAGGACAGGGGGATGTTGAGTGAATGGGGCTCAG |
| | CCCTGTAGCCAGTCAACATACAGAGGCTTAAAGCAAATTTCCAAAAGGCATCCCTTTTCCTT |
| | CAAGATACTACCACCCTCTATTGGACAACTGCCCTGTTAATAACTGTACAAATCTAT |
| SEQ ID NO: 320 | AAGAGGTGGGGGCAGGGTGAGATCCAGGTTTTGCCAGGCTTAAAGTTTATGCAATTTTGGG |
| | ATCCCTCTTTAAGAAACAGAATACAGAATTACAAATAATTTTGAAAATTAAGTCCAAGGCC |
| | TTGAAATGTGCTGGAGAGTGACCCTGAAGCTTAACTTCACTAGTTTCATGGTAAATCAACTT |
| | TGGGAGAGAAAAAGCTTCCCAGGAAAAGGGATGGCGTGTTCAGCGGGGCTGGAGTTTGGA |
| | GGGATGCACACTAGACCATTAGACTTGAGAATCCTGGGTTCTCATGCTCTGGTTTC |
| SEQ ID NO: 321 | ATCACGCCAGATGCATTCCAGTCTGGGCGACAGAGACAGATTCCGTCTCAAAAAAATTATT |
| | ATTATTATTATTGTTATTATTATTTTCCCTTGAGATGGAGTCTTGCTGTCATCCAGGCTGGAG |
| | TGCAGTGGCGTGATCTCGAACTCCTGATCTCAGGTGATCCAGCCACCTTAGCCTCCCAAAGT |
| | GCTAGGATTACAGGTGTGAGCCACTGCACCCGGCCAAAAAAATATTTCTTATGGCATTATCT |
| | GCAAAAGTTGAATATTGGAGACAACCCAAATGTCAATTAATAGGGGACTGAT |
| SEQ ID NO: 322 | TCCAGTACTGCTTATCCATGGATAATGAGGACCTCCTGTAGCTGTAGTCACTTTGGTGCATC |
| | GTTTCCAAAAACCTAATGAGTTGTGATGTCAATTGCAGCTGTCAATCTACTTTTAATCACAG |
| | GCCTCAAATGAAGAAATTCATTTAGAGGATAAGCAAATTACAGCTTCAGTTTTGTTCATAAA |
| | CTTAAGCCCCTTATTGCTAATTTTTTTGAAAGAACAAGTTTATGCTGATTTGTCCAAGGATAC |
| | TCAGCAAATATTTGGAAAATAGTTCAAAGTTTAATGTATAATCATAATCCT |
| SEQ ID NO: 323 | CTCATAAAGTTATTAGTGGGGAACCCATAGCAGGGAGAGGAACAGAGAGTGTATCAGACTT |
| | CAACATGTATGTTGTGACTCTTGTTACTTTAGAAGAAAATGAACCTCACTATGTAATAACCT |
| | AACTCTGTTGTGTTTATTCATTTTCTTTCTTTTTTTATTTTTATGTGTTTATTCATTTTCTTCTA |
| | AGTTCAAAGGTCATTTCTGGGTAGGAGAGCAAATGTCCTTATATTTTTTCCAAATAGACCAT |
| | TTGCTTGGTGGGCCTCACCCCGGTGGAATGGAAGTACATTGGCAGCAACC |
| SEQ ID NO: 324 | TATATATGTATATATGTGTATATATGTATATATGTGTATATATATGTATATATGTATATATAT |
| | ATGTATATATATGCGTATATATATGTGTATATATGTGTATATATATGTATATATGTATATATA |
| | TATGTATATATATGTGTATATATGTATATATATATGTGTATATATGTATATATATGTGTATAT |
| | ATATGTATATATATATGTGTATATATACATATATATATAAAATATAGAAAGCAACAAAAGTG |
| | AGATACAGTTCTTCCTCTTCCCTTTCTTTTTCTTAAAAGAAAATCTTTT |
| SEQ ID NO: 325 | AATCATACCTAATAAAGTTTTTATCAGTGTGTTAGATGGCCTTCAAAATATGGCAGAGAAAT |
| | CCATAACCAGGCAAAATAATTTTGGGTCTCATTTCTTCTTATAAGTTATTACTTGCCTATAAC |
| | AAACTAATCTAAAATATGCCTTTCTGCTCTAAAAGCTAAAGCTTAAGAGTCCCTTGGCAGAT |
| | GGGACTTTTAAATTATTCCTGTAGAAAATTGGTAGTATATAAGTGTTTTAATTTATTATGTGT |
| | TTTAATTTATTCCTGTAGAAAATTATTCCTGTAGAAAATCGGTAGTATAT |
| SEQ ID NO: 326 | GTAATCCCAGCACTTTGGGAGGCCGAGGCGGGCGGATCACGAGGTCAGGAGATCGAGACC |
| | ATCTTGGCTAACATGGTGAAACCCCGTCTCTACTAAAAATACAAAAAATTAGCTGGGTGTG |
| | GTGGCGGGCGCCTGTAGTCCCAGCTACTCGGGAGGCTGAGGCAGGCGACTGGCCTGAACCC |
| | GGGAGGCGGAGCTTGCAGTGAGCTGAGATCAGGCCACTGCACTCCAGCCTGGGTAACAGAG |
| | CGAGACTCCGTCTCAATAAAAAAACAACAACAACAAAAACAAAACAAAACAAAAAAA |
| SEQ ID NO: 327 | TGGCCAGTATTCTGCCTAGCCACATCAACAAATGTCTCAGATTCCTCAAAAGATTTTGACAT |
| | CACCAAGAGGGGCTTGGAGTATGTAGATGAACACAATGCCTTCTGGTTGGTCTTGTTCATAT |
| | TGTACATACATTTCTTAGGATTTTGCAAATATTCTAGTATCCAAAATGTTATTATCTGGCTCT |
| | TAAAATTTGAAGTCTTTATGTCACAAGTGACAGGCTAGAGCTTGTGCAAAGTGAATGACCCT |
| | ATGTGTAAAGTGTTTGGAATCAAGGGTGAGATGCCAGGCCACAATAAACTC |
| SEQ ID NO: 328 | TTCAGTTTCTGCCAGCATCGTCTGCATTCAAACCAATGACCTCGAGGTGAAAATCTCCAGAT |
| | AACATGATTGGTCCCCATGCTCTCATGATTTCCTTACTTAAGCAATTAAAGGCCAAAGCAAG |
| | ATGCTTAGTAACTCCTTTTCCACATAGAGGCACAAATAAAGGAAAGCTGGTCACAAGTTTTG |
| | AACCAAAGAGTTAAAAGAAGAATCATAACTTGTCTCTATTTAATGATTGGAAACTCTCACA |
| | GACTTCTGTGAAGTCTCTCCCCCTTTTGTACCTTTCTTACAAGTGATGACTAT |
| SEQ ID NO: 329 | CCTGAGATTTTAGGAGCTTTTGTAGAATTTCTGAATTTGTAGCTGGTGACTGAGTAGAGGAG |
| | CTAAGTTCCACTCCAGAGCTCCAGAGTGAAAGGTGTGCCTTAAAGAGGGGAAAGGGGAATC |
| | AATTTTCCCCTGAAGCCCCAGTACAGGCTTGTTGTGAGGATCAAAAGAGCAGGAGGGTATG |
| | TCCTGAGGTCCCCAGATGCATGTGTGATGCTGCTACTACAGGGGTCAGCTGTGCAGAGCAC |
| | CCCCACCCCCACCATTTCCATTCACACCCTTTGGGTTCTATAGAGTTCCACTCCT |
| SEQ ID NO: 330 | CCCTGGAAATTAGCGCCTTGAATAGGCACCCCTAGCAGAAGAGACAACAGGCCGCCTATAG |
| | TAAACTCAGAGCCCCAAATCATGTGAAGCAAGCATCAGCCTGAGGGTCTGTGAATCCCAGT |
| | TCTAGATCCCAGTTCATTAAGGATTTTCTTTCTGGCTTTGCAGGGAAGGAGTGTGGTTTCCA |
| | GGAAAGTCCCTGATATCTGGAGAAGAAGGCTCCATGTTGGCCCCAGAATGGCATACTCTTG |
| | GCTCCAAAGACATTAAGTCTTCATCTTCCCACACTAAGAAACAGGAAAATTTTTA |
| SEQ ID NO: 331 | TGAGCTGTGATCGTACTGCTGCACTGCAGCCTGGGCAACAGAGCAAGATCCCAGCTCTCAA |
| | AAAAAAAGTAACAGAAGAAACCTACGGACTCTGACTCAGAGTTGTTGCTCATTCAGCCCTG |
| | GTCATGGATTGAACAGTGTCCCCTGCCTCTCCTGAGAAAAAAAGAGTCCTCTTCTCCAGTAC |
| | CTGTGAATGTGGCCTTATTTGGAAATAGGGTTCTCGCAGTTGATCAAGCTAAGATGAGGTCA |
| | TTAGGGTGGGCCCTAATCCAAAGTAATTGTGTTCTTATAATAAGGACAAATTAT |
| SEQ ID NO: 332 | AGGAGTGCTTGGGTCTGGGAGGTTGAGGCTGTAGTGAACCATAATAGAGCCAATGCACTCC |
| | AGCCTCGGTGACAGTGCAGGATCCTATCTCAAAAAAAAAAAAAAAAAAAAAAAATTTTCAT |
| | CAATTAGAGAAAAGTTTCTGGGATTTAATTTATTCTGTTAAGATGCTCTCCTCCTCCTGGAA |
| | AGAAAGAGATAAAAACAAGTCTGGGAAAATGCTGATTGTTAAAGTTGGGATATAAGTACAT |
| | GAGAGTTCATTATACCATTCTTTCCAGACTTGGATGAGTATAAACATTTTTAACA |
| SEQ ID NO: 333 | GAAAGCAACCATTCATTCATTCATTTAGATTAAAAAAATAAATAGAGACAAAGTCTCACCA |
| | TGTTGCCCAGGCTGGTCTCCAACTCCTGACATCCAGCAATTCTCCTGCCTTGGCCTCCCAAA |
| | GTGCTGGGAATACAAGTGTGAGCCACCATACCCAGCCAACAATTCTTTAGTTTAAAATGTTA |
| | AACTGAATAAAGTCCTTAGTCATCTTTCCCCTAAGATTTTAATTAAATTTATTCAGAAAAAC |
| | TTAATTATAGAAACCAAAGCTCTAAGAGATTATGGCTGTCAAAAACAAATTCT |
| SEQ ID NO: 334 | ATCCAAGCAGCATGAAACTGCACTCCTAGTTAGGTTTGTGGATTATAAATGCTCATAAATTC |
| | CTTATCCCGTCCCTCATGGAAAGGTGGGGTTTACTTCTCCACCCCCTTAATTCTGGACTTGGC |
| | CTATGACTTGCTTTGAACCAGTAGAATATGGTGGAAATGAAGCTGTGCCCAATTCTGGGCTT |
| | ACCCTTTAAAACAGAACTGGCAGCTTCTGTTTTCTTCTCTTGGAATTCAGCCACCATGCTGTG |
| | AGGAAGCCCAAGCAGTCCTGTGGGGAGGCCCATGTAGAGGAGAATCAAAG |
| SEQ ID NO: 335 | AATACAAAAAATTAGCAGGGTGTGGTGGTGCGTGCCTGTAGTCCCAGCTATTTGGGAGGCT |
| | GAGGCAGGAGAATCACTTGAACCTGGGAGGCGGAGGTTTCAGTGAGCTAAGATCACACCAC |
| | TGCACTCCAGCCTGTTGACAGAGTGAGACTCTGTCTCAAAAAACAAAATAAAACAACAACA |
| | AAAAAACGAGATGATGTGAACGGACACCATACCAAGGAAGGTATATAGATGATAAGTTAG |
| | CAAATAAAATATGCTCAATGTCATTGGTCATTAGAGAAATGCAAATTTAAACCACAA |
| SEQ ID NO: 336 | TTGCAGAGCCCTGGGTGGGGCCTGTGACTCTGAGATCTCCAGGAGCTTGACAAGCTGGGAA |
| | GAGATGAGAAGTCATTGGCTGGCGAGGAAGGGCAGGGGGACGAGGTAGTGACAACAGTCA |
| | AATGGGCTCCAGCCAGAGGCAGTGAATACACCAGTGCTGAGGATCTGTGCTAGGAACAGCA |
| | AACAGATACCAAGGTTGGGGAGAGGGATCTAAGTCACTGAGAAAAGAATGAGGCCTGGTA |
| | GTGAGGAGCTTGGTTCAGGCATCCTCTTTGCATGAAGTCAGAACTGAAACCTAGGCGT |
| SEQ ID NO: 337 | CAAGAAGACATGACACCTGTGGCCTTCACACAGCAGGTCCCACCTGCTCGGTGACACACAG |
| | TCATTTCAGGGTCCACAGCGTCCCACTGTCCCATGTCACTGGGTGTCACAGGGCCACACACA |
| | CAGTGATCTCTCACTGTCACCCTCACTTGGAGTCTCCCATATCTTCAGTGTTGCACCCATGTT |
| | CCCAAAGCCACCATGTCACCCTCACACAGCATCAGACACAGTTCCACACTGGCTCTGTGTCA |
| | CTGTTACATTTCAGCATCATCCAGGACCTCCTAGCCTTAAAAAACAAAAATC |
| SEQ ID NO: 338 | ATTCTCAAATAGCATTTTATACTATTTCTTAATTTTTTATTAAACAATATCTGACTTCCTACTA |
| | TGGAAGATGAAGATGTAGCTCCCCAACAGCTTATTCCACTATCACAAAACACACCCACCTA |
| | CACGTGTTGCTTCCCCAATATTCACAATAGCGTTGCATCACAATATTTAGTGTTCTCATTTCT |
| | ATGCCTATTTAAATATTATTTATAGTTGAGCCATGTAGAACAACGTGATTATATTTGTACCAT |
| | ATGTTGTTCTCCCTGGAGTTAATAATTGCCTCACAGAATGTACATAAAT |
| SEQ ID NO: 339 | ATTTAATCTGACTGAATACTAATCTGTCCAAGACTTCCACCAGCAGTTCATTTTTCCACCAG |
| | CTTCTCATATTTCCACTTTACCACCAAACCTGACATTATCCAAGGTCTAAATGTTGCCAGTGT |
| | GGTACATGTAGTGATAGCAACAGTGTAGTGTGTGTGTGTGTGTGTGTGTGTGTGTTTTAGTG |
| | TAATAGTGTAGTGATAGTGTAGTGGTTTATTTTACATTATAATACTAATAAATGGAAGCATG |
| | TCTTCATATGCATGTTAACCCTATGGAGTTTCTCTTCAAAATACCTCTTCA |
| SEQ ID NO: 340 | AAATCTTTATTAAGGAAGGCCTAACAGCTGTATCAGATAAGTTTGCTTTTTCTTGGAAACTT |
| | TGGTAAAAAAAAAAAATCACCTGGGAAATAATGTGATTAGAGATGCTTCTTAAATGTGCAT |
| | ATGTGTTTAGTCTGTTCTACAAAGATATTATTTTCTTTGTAGCATTGTTTTATTGAACAAATT |
| | TTGTTAAATTTTCTATTAAAATAGTCTTATAGTGATTTAATAACAATTTGGAAAAGTCGCAA |
| | TCTTTATAAGCATATGTGTAAGTGAGATGGATTACTATTAAAATGCAATTTC |
| SEQ ID NO: 341 | CAGATTTAGGGTGAGCCGAGTGAGGCAGGGTCATGTGAATGCAGGGTAAAATCCTCTATCT |
| | TTATTTATATTGTTGATATTTTGTTCATCATGGATAGTTGTGTGAATTTCTACTTTTAAAATAT |
| | TGCATAAAGGTATTATTCATCTTGATTTCCAAAGTCTTGGTGCCTCCTTAAGTTTTGCACCTG |
| | GAGCACGTGCCTCACTCACTTCATTCTCATCCACCACTCAGCTATACAGTTGGAAAAGCATG |
| | TGATGATATGGACAGCACCTAGTATAAAGAGAAGCTTGAAAGAATTTTGA |
| SEQ ID NO: 342 | GGCTCATGCCTGTAATCCCAGCACTTTGGGAGGCTGAGGCAGGTGGATCACGAGGTAAGGA |
| | GATGAAGACCATCCTAGCTAACACGGTGAAACCCCGTCTCTACTAAAAAAAAAAAAAGTTA |
| | TACAAAAAATTAGCCGGGCGTGGTGGCCGACGCTGTAGTCCCAGCTACACGGGAGGCTGAG |
| | GCAGGAGAAGGGCGTGAACCTGGGAGGCGGAGCTTGCAGTGAGCTGAGATAGTGCCACTG |
| | CACTCCAGCCTGGGAGAGACGCCGTCTCAAAAAAAAAAAAAAAGATTTATAGAGCTA |
| SEQ ID NO: 343 | ACGAGAGATGAATTTCTGGGCCTCAGCCTGCCATTTACTTGATTCATGACTGGGCAAATCTC |
| | AGCTTTTTCAAAACCTTAAATTTCTTCTTTATTAAATGAAGGATTAGTCTAAATGACCTTCAA |
| | GGTCCATCTCAGCTGCAAAATGCTAGAATTCTATGACCATGCATTTCTAGTTATTTAAATTA |
| | TTATAGAAAAATACATTTGAAGCATGTAAGATTATCGCAACCTGTAGCTTTCATAATTGTCA |
| | CTAAGGAAAGCCATAGGATTCTCAGCTAATGATAAAATGATCGAGGTAAAA |
| SEQ ID NO: 344 | AGTTTTAATGAAGAAGGAAATCCTGCCATTTGTGACAGCATAGGTGAACCTGGAGGACATA |
| | ATGCTAAGTGAAATAGATCATATAAAGTACACATACTACATGATACCACTTATGTAATTAAT |
| | CTAAAATAGTCAAACACATAAAAGCCAAAAGTGGAATAGTGTTTCCAGGGACTGGAAGGG |
| | AGAGGAGAACAGGGAGGTATTAGTGAAAATGTACAAAATTTCAGTTACACAAGATGAATA |
| | AAAAGTCCTAGAAATTGACTCTATAGTATAATGCCTATAGTTAAGAATCTTGTATTA |
| SEQ ID NO: 345 | AGGTGTCATGGCTGACACCTGTAATTCCAGCACTTTAGGAGGCTGAGGTGGGTAGATCACTT |
| | GAGGCCAGGAGTTTGAGACCAGCCTGGCCAACATGGTGAAACCCCATCTCTACTAAAAATA |
| | TTTTAAAAATTAGTTGGGCGTTGTGGTGTGCACCTGTAATGCCAGCTATTGGGGAGGCTTTA |
| | GGTGGGAGGATAGTCTGAGCCATTGGAGATCAAGGCTGCAGTGAGTCGTGATCTGATTGCA |
| | TGACTGCTCTCCAGCCTGGGTGACAGAGCAAGACTGTCTCAAAAAAAAAAAAAA |
| SEQ ID NO: 346 | CCCATGCCTTAAATATGCTTCTTATCCTCTCACAGTCAAAAAGGATTTTTACCTTGGTACAGT |
| | CTGATTCCTGCAACACCTCTCTAGTTATTGAATGTATAGTAGTTTGCTATAGACAGACTACTT |
| | TGCAGTGGCTATCAAACCTTTCTGAGACTCATTCTAAGAAATACATTTACATTGTGACTTAG |
| | TATATGCACACATAAAAAGAAGAAAGTTTCACAAAATAGTTTCCTTACTAGCAATGAGCTG |
| | TTTTTTGTTTTATTTCGTTAACAACAACAACAACAACAACAAAAACTGCTA |
| SEQ ID NO: 347 | CTCTGGGCCTTTACTGCCGAATCCAGGTCTCCGGGCTTAACAACAACGAAGGGGCTGTGACT |
| | GGCTGCTTTCTCAACCAATCAGCACCGAACTCATTTGCATGGGCTGAGAACAAATGTTCGCG |
| | AACTCTAGAAATGAATGACTTAAGTAAGTTCCTTAGAATATTATTTTTCCTACTGAAAGTTA |
| | CCACATGCGTCGTTGTTTATACAGTAATAGGAACAAGAAAAAAGTCACCTAAGCTCACCCT |
| | CATCAATTGTGGAGTTCCTTTATATCCCATCTTCTCTCCAAACACATACGCAG |
| SEQ ID NO: 348 | ATGAAGCACTCTAAGAAAATTCAGCTGGGCAGGTATTTTAAGGTCACTGACTGACTCCAAA |
| | GTGAAGGTAGAACCCATATTGACCGCAATTCATCACACTTCTGTAACTTCAGAACTCACTGC |
| | TGACTGGTGGAGAAGGTTGCAATTGCATTCAGTCCGAATCTCTGAACATATTATTTAGAAAT |
| | ATTAGCTTATCCAGCTGCCTACGGAGCTTCCCATCTGGGCTTTCAGTTGCTACATTCTCTAAA |
| | GCCGTATCTGACCCACACCTTACTAACACTATGTGCTTCTACAGCATTCACC |
| SEQ ID NO: 349 | GGGAAAAGACTCCCCAGTCAATAAATGGTGCTGGGAAAACTGGCTAGCCATATGCAGAAG |
| | ATTGAAGCTGGACCCCTTCCTTACACTGTATACAAAATCAACTCAAGAGGGTTAAGGACTTA |
| | AATGTAAAACCTAAAACTGTAAAAACCCTGGGAGACAACCTAGGCAATACCATCCTGGACA |
| | TAGGAACGGGCAAAGATTTCATAACAAAAATGCCAAAAGCAATTGTAAGAAAAGCGAAAA |
| | TTGAAAAATGGGATCTAATCAAACTTAAGAGCTTCTGTTACAGCAAAGGAAACTACC |
| SEQ ID NO: 350 | ACGCTTCAAGGCTGCCAAATATCACAATTTATGTAACTGCAGTAAGAGTAGCATATATTTCC |
| | ATAGCACTTATGTGCCAATCATATTCTGAGCACTTTATGTTTATTAACTTATTTAGCCCTCTC |
| | GACAACCTACGAGGTAGAACTATTATTTCTATTTTACAAAATAGGAACTAGGCACAGATAG |
| | GGTAAGACAGTAGTTCAAGATCACAGCCACTGAACAGCAGAACCAGGATTTGAACCCAGGC |
| | AATCTGTGTTCTTCGGGCTCTGTGTTCTCAACCATTATTAAACAGTCTTATTA |
| SEQ ID NO: 351 | GACGATGAAATACAAGGTTAATTTTCTTGACCAACCATATCTAGTAAATGGCAGAGCTCTA |
| | ATTTAAACCTGGAGCTCCAGCTGTACAGTCTTAAAAAATACTTCAAATATTTTATTGATTCT |
| | CCAGAATGTGTCTGTTTTTGTTTGTATAACTGGAGAACATTCCAAAGCATTTCATTAATTTTT |
| | TAAAAGAAAATATTAGCTGAACATTAACATCAGTGATCTTTAAAAGAAAGAATAATGTATG |
| | AGTTTAGGAATTAGTTATTTTAATAATAGAGTAGGAGTCCAAATTTGCTTTAA |
| SEQ ID NO: 352 | GCTGGGCGCAGTGGCTCATGTCTATAGTCCTAGCACTTTGGGAGGCTGAGGCAGGAGGATC |
| | ACGAGTACAAGAGATCCAGACCATCGTGGCCAACAAGGTGAAACCCCGTCTCTAGTAAAAA |
| | TACAAAAATTAGCTGAGCGTGGTGGCATGTGCCTGTAGTCCCAGCTACTCGGGAGGCTGAG |
| | GCAGGAGAATTGCTTGAACCCGGGAGGCAGAGGTTGCAGTGAGCCAAGATTGCGCCACTGC |
| | ACTCCAGTCTGGCAACAGAGAGAGGCTCCGTCCAAAAAAAAAAAAAAAAGTCTGGT |
| SEQ ID NO: 353 | AGCTTAAAGATGTGGTAGCAGCTGCAGACCAGTACAGCGTCCTACAGACGCCCACAAAGGC |
| | CTGCATGTAGGGCATCAGGGCATTCCCAAATGAACCATGACGTGCAGAGTGGCACCGAGTT |
| | CGGGAAAACCAAGAACCGAGTGTTGGGTTCCGATCTGCCAGGGTTATAATATCCTCTGGCA |
| | AATGGCCCAGATGCAATCTGTACAAAATGGAATTAAGACGAAGCCCTCCCTGCTGAATGCC |
| | TGAGAGGAACCAAAACATCACTTCCATTTTTATTTCAGGGCAAAAACTATATTAGA |
| SEQ ID NO: 354 | GCCAAGGCAGGAGGATCACTTGAGGCCAGGAGTTTGAGACCAGCCTGGGCAATATTGTGAG |
| | ACTGTGTCTCTCCAAAATATTTTTTAAGAAATTGGCCAAGCATAGTGGCATGTGGCCAAGCT |
| | GCTCTGGAGGCTGAGGCAGGAGGATCACTTGACCCTAGGAGTTCAAGGGTGCAGTAAGCCA |
| | AGGTCACACCACTGCACTCCAGCCTGGATGACAGAGTGAAACCCTGTCTCAAATAAATAAA |
| | ATACATATTTATCCTTAAAATCACATAGTGCAATTGTATTTACAAACAAAAGTCC |
| SEQ ID NO: 355 | AAGGCATGGCAGAGAGAGGCCACCCATCCAGGGCCACACAGCTGGTCATCTGCAGAAGCA |
| | GGGTCTGTGCCCACGTAGACCACCTGTTGTGACCTCTTCCTTTCTATAGCTAAATCCATTCGT |
| | CTTCTCAACAGGTATGAAATACATCCTTATTTTTGCATGAAGAAACCAAGGCTCAGAGAGG |
| | CTTATGGGTGGCCCAGATAATTGTGAATTAGCACATTGGGATCCAGCTCCATTTCACTTTTG |
| | AAACCCAACTTCTTTACAATATGCTGTTCTGCCTCCCTGTAAGGTCTGAGAATT |
| SEQ ID NO: 356 | CTCCCAAATTGCTGGGATTACAGGCATGAGCCACCGCACCCGGCCCACTTTCACCAGTTCTG |
| | TTCATCATTGAACTGGAAGATTCTATCTTTGGCAGTTTGCAAAACAAATAAACTTAAAACTG |
| | GAGTTAAAGACATCCAGATTAGAAAGGAACAGTTTAAACCATCTTTTTTCAGATGACATGA |
| | TCTTGTACATAGAAAATCCTAAGGAATCAACTTTAAAACTGTTAGAATAAATGAGGTTGTA |
| | GAGTAAAAAAAAATCAATATACAAAAATCAATAGTGTAGTGCAGAAATATATTA |
| SEQ ID NO: 357 | GAGCATCCTCGGAAACACCTGGAAACTTCAACATACAAAAAGCCCAGGCGCCACCCTGGAC |
| | CAAATGCATCAGAATCTGCATTTTTACAAGATCCCCAGGTGACTCATATGCACATGAATGTT |
| | TGAGAAACATTGCTGCAGACAATGGGGAGCCATTGGAGGTCCCTGGGTGAGAGGAGTAATC |
| | TCTTCTTAAATGTGCTTGGGAAATGCAACTTTAGGAAGCACAGTAGAGGGCACACAGAATG |
| | ACCAAATGAATGAACAGTTATTCAACCAACAAAGTCTTTTGAAAATATGAATGTT |
| SEQ ID NO: 358 | CAGGCATCAGTTACATAACTTGTATGACAGGGAACAGTCTTGACTTGGGTATTTCCAAATAA |
| | GCCAAGTTTTACACACAAAATGAAGTATATTTAGTGGATTGATGATTGAATAGGGAAGTAC |
| | TACTGAGAAATCATCACCTAGGGGAACCTCATCTTTTCAATGTGTGGGATAAAGATTCCCAG |
| | AGGGTATAGGATTGATATTGTTGATGAAAGGATGAGAGAGAGGAAGGCTGTGAGTTACAAC |
| | AAAATTCCAGGGAAGTTAGAACAACTTTAAAAAGACTTAAAAATAGAAGAAATG |
| SEQ ID NO: 359 | TAATCTTTTAAATTCACTCAATAGTATATTATTGAGATCTTTCCATGTCAATACCTGATGTCA |
| | TTTATAATGACGGTAGGATAGTTTTCCTTTATAAGTATGTACCTTTATTTAGCCAAGATTGAC |
| | TTTTAAGATGTTTCCATATTTCATTACTATAAATAGTATTTAGAAAACTTCATCCTAATTGTG |
| | ATTTGTTTACCACTGCAGCCAGATATAAAATACGTTTTCTTAGCTGGGAGACTCACTTTTGC |
| | ATAGAGGTATAAGTATAAGTATATTTTTAGGTGAAGTTCACATAAGTTA |
| SEQ ID NO: 360 | CTTGATTTTTATCTAGAGTCTATATAGGACAAACCATATAAAACTATCATTGGAAACCATCT |
| | ACATATTTTGTTCAATGTCCCTTACACATAATTTTTTCCTTTGAAAAAGTAATCCACATCTTT |
| | TACAAGATTATAGGAGTTATAAAATCCATTCTGTTTACCTTGTTACTGGCTATCAGGAATTT |
| | CTGTGTGGGTTTAATCACAGTTAATATATTTGCTCCTGTAATCATATATTTTCGACTTCCAAA |
| | AAAAAAAAAGCATTTTTTTTCTCTATTCTAAACGGTTTTTTGAAAAAAAA |
| SEQ ID NO: 361 | CCACGCCGTCGGTAGGTCCCCGGTCCGGGAGCGGGAGAGACCGGAGCGCCGGGGACGACC |
| | CCGGCAAGGGCGTGGCGTATGCAGATGAGCACGGCGCGCGGAGGCTGCCGGTCACGCTCG |
| | GCCTGGGACCCCATGTCCCGCGCGCGATATCCGAGTGCTGTCGGGGTGTCTTCCCCTAGCGG |
| | CAACGGTGGAGCGAGGGGGCTGGAGAGGGTGAGGGGGCTGGGGCCGCACGAGAGAAGTG |
| | ACCGTGTGTTGAGAGTGTGGTGGGCGCGAGGGTATGAGAGGAAGCCGCACGGCCAGTCC |
| SEQ ID NO: 362 | GCAAGTCATTTGGCCTCTATGAGGCCAGTTCCTGCAATTTTAAAATAAAATCAGTAGATTTC |
| | ACTTTACAAAGAGGTTGCAGTAATCAACATAGATAATGTATATAGAAAGTGTCTTGTAAAC |
| | TGATCGTTACTGTATATAAGGTTATTATTACTATGACTTCTTTTTCCAAGCATTGCAAAAATC |
| | GTAGAAGACATTTTTGTTGGTATCTATAGTTATAAATATGTATATATAGTATATTGATTAGAT |
| | TATTCTAAAATCTTATTCAAGTCTACTAATTTGATGTTACTTAAATCTTAA |
| SEQ ID NO: 363 | ATAAGGAGGTGCTTTTTTAAAATTGTTCATAGACTTCTGTAAAATGCAAGATAAATTAAAGT |
| | TATTATAACAGTGAAAAAAATCTGATTGATGCCTTTTTCCCCCCAACAATGATTGGAAATAT |
| | AGATGTCGTACTGGTTCGAAATATTTTTTTTTTAAGTTCTCAGGTCTTGAAATCTCCAATCCC |
| | ATCTGCACATTCACCATTTAGACATCTTGGTAAGTGTTGACTTGCCCCTAATATTTTGATGTT |
| | TATAGTAATGAATATACTAGTGTAAATTCTCAATCAGAATGGAATATTCT |
| SEQ ID NO: 364 | ATATGATCCTTACCTTAAAGACACTGGCGATTTCATGGGCACAGAAACAAACATGTGTGAA |
| | GTACTGGGATAGCTTAGGAAGCCGATCCCCCACGTGTCCCATTTACCAGAGTGAATCAGAG |
| | TGCAGGTGATGCGGAATATTATCTTCCATGCTATTCCTGAAATATTTCCATCTGTCCCTGCCT |
| | CTCTGTTACCACTGAAATGTTCCTTTTTTTAATTCCTTATCATCTTTTCACCTATTAAAGTAGC |
| | CTCTTAAGTGGTCTCCAGAAGAGAAGCTCCAGATCTACTGCAGGCTACTCC |
| SEQ ID NO: 365 | TCAGATAAAAGCATTTATTTTCAAACAATACTCACTAAAACTTTTGTACTTTTAAATATTTAC |
| | TCCAAGCCTTCTGCAAATAAAGAAGTATTTTCCCTCAGTAAATCAAATTCTCTGTTGAGTTT |
| | CTGAATTGCTTTCATTACTTATACATATTAGAATTTCATTCCAGGATTTCATTGTGAAAAAAT |
| | TATTGAAAAGTATTTGAAAATTGTTCTATTAAAACAAAGATGCATGTGATAGTAGAAAAAA |
| | AGCATACATTCATTTGCTTTTACAGGTCAATGAAAATTTGAAAGAAATTTT |
| SEQ ID NO: 366 | ACCGTCGCCCAGGCTGGAGTGCAGTAGCGTGACCTCAGCTCACTGCAACCTCCGCCTCCCA |
| | GGTTCAAGGGATTCTCCTGCCTCAGTCTCCTGAGCAGCTGGGATTACAGGCGTGCGCCACCA |
| | CACTCGGCTCATTTTTGTATTTTTAGTAGAGACGGGGTTTCACCATGTTGGCTAGGCTGGTTT |
| | CAAACTCCTGACCTCAAGTGATCCACCCACCTTGGCCTCCCAAAGTGCTGGGATTATAGGCG |
| | TGAGCCACTGTGCCCAGCCCAGTTGTATTCTTTATGATGGTCAAAATTTCCA |
| SEQ ID NO: 367 | CTTACATACAACTGAGTTTTTGCTTACACATTCTTTAATTTCTTTTAATTCCTATTTCAGTCTG |
| | ACTGAAATTAGAGGAGCTACCTGGCTGCATACATTACTGATTCCCCGCCAACGTGTTTCTAT |
| | GTTCTTTGAAAGACAATGTAATTGTGCCTTACTTGAAGTAAATTGATGTGATCTCCACAGTC |
| | ATCCCATAAAATATTCACTATCATTATCACTATCATTATAATCTGGCTCAAAGGCCAGATTA |
| | AGATGGTCAAATAAATTAGTTTGTTAAACCAATTTAAAACACACAGTCTT |
| SEQ ID NO: 368 | TCTCCACTGTCCCTGTCAAACTCCCAGCAAATGTGCACAGGGAGGGTGGGGATGGCTCCAT |
| | CTTCCAGGGTGACTGTGGGTGAGCATCCTACAGCTCAGAGGCTGCATCCTGCAGGGCGAAT |
| | CCACCCATTTCCCTGGCTTCTCCAAACACCTCAGCCTCTTGGTCTTCTACCCTTTGCCCTCTG |
| | CAAGAGAGGGAGGAAGGTGTGGTCAGCATGGACTTTACCCCTGAAGGGCTATTGCTTTAAG |
| | GAGTGTTGAGGATTGGGAAGACATCTGTTAAGCCAGCTTGGCAAATTCCCTGTC |
| SEQ ID NO: 369 | TGCAGTAATAGAGCATGGATCTGAAGAGGTGTGATTCATTTTCCTTAGGTGTCTGCCTCTGC |
| | TTTTCATTTAAAAAAAAAAAAACTCAGCTTCTATTTCTAACACAAACTTTCACCTCCTATGG |
| | CTTAAATAGATGTCATAAAAATTTAGGATTTATATAGGGAATGTACACACTTAAGTCAGCA |
| | AAAACTAAATTAATTTTGCCCTATATTGTGCTGGTGAACCATGATATACCTTATAATCTTGTC |
| | AAAAAGAAAAGTTGTAGCTGTGTTCACAGGAAAAAAAAAAAAAGAAAAACAC |
| SEQ ID NO: 370 | GGTCAGGAGTTTTGAGACCAGCCTGGCCAACATGGTGAAACCCAGTCTCTACTAAAAATAC |
| | AAATTAGCCGGGTGTGGTGGCACATGCCTGTAATCCCAACTACTTGGGAGGCTGAGGCAGG |
| | AGAATCGCTTGAACCCAGGAAGCGGATGTTGCAGTGAGCCGACACCACAACACTGCACTCC |
| | AGCCTGGACAACAAGAGCGAAACTCAGTCTGAAAAAAAAAAAAAAAAAACTTTACAGAAA |
| | ATTTCAAACATACACAAAAGTAGAGAGAGCCTCTCACCCAGACTAAAAATGATCAGC |
| SEQ ID NO: 371 | TGGCTGACCTGTCTGTCCTCCCATCCAGCCAGAGGGACACAAAGACCTCTTTGTCTCTCCAC |
| | CCTTCCGCCCAGAGCCTCCCCAGCCAGTGCAGCTCTGAGCGTGTCCCCCAGGGAGGGAGAC |
| | AGCCCCTGGTGGGGACTGGCAGTCCTGGCAGCTGCCCTGAGGCACAGGCCCTGGGGGACTC |
| | GGTGCTTTGAGGGCAGGGGCAGGTGATGGAGATCGGCTGGGGAAAGGGAGGCCGAGAAGG |
| | CGGCAGTGTGGAGCGTGTGAATTTGTTCATGAACTTAGCTACAAAAATGTCTTTTC |
| SEQ ID NO: 372 | GCTCGTGCCTGTAATCTCAGCACGTTGGAAGGCCGAGGCAGGTGGATCATCTGAGGTCAGG |
| | AATTCAAGACCAGCCTGGCCAACGTGGTGAAACACCGTCTCTACTGAAAATACAAAAATTA |
| | GCCGGGCGTGGTGGCGCATGCCTGTAATCCCAGCTACTCGGGAGGCTGAGGCAGGAGAATT |
| | GCTTGAACCTGGTAGGCGGAGGCTGCAGTGAGCCAAGATCGCGCCACTGCACTCCAGCCTG |
| | GGCAACAATGCGAGACTCTTTCTCAAAAAAAAAAAAAAAAAAAAATTTTTTTTTGT |
| SEQ ID NO: 373 | AAGGAGTCTTAAGAAGCTGACATTTTAAAAACAAAAAAAAAGTTGTTATTATAGTATTGTG |
| | ACATTCTGCATAATTTCCTAATGAAGACCAGCATTCATAGTGCTGAGGAACAGACTTTTCCA |
| | GGCATTTTACTGAATAAGCTGTAAAATGCTGCCTTTGACTTTGCTGGTGGCCTGCTTGGCAT |
| | AGCTTCTCTAGGTCTCTGCTTAACGTTCCCAGACAGCACAGCACTTCGAAACACTCTCCCCA |
| | CTGAATACAACTAACAAGTTCTTGATAAATCTCTACTATCAAAACTTATTTGG |
| SEQ ID NO: 374 | GAATCTTGCTCTATTGCTCAGGCTGGAGTGCAGTGGCGAGATCTCAGCTCACTGCAACGCCT |
| | GCCTCCCAGGTTCAAGCAATTCTCCTGTCTCAGCCTCCCGAGTAGCTGGGACTAAAGGTGCG |
| | CGACACCATGCCGGCTAATTTTTTGTATTTTTAGTAGAGATGGTGTTTCACCATGTTGGTCAG |
| | GCTGGTCTCAAACTCCAGACCTCAGGTGATCTGCCCACCTCTGCCTCCCAAAATGCTGGGAT |
| | TGCAGGAGTGAGCCACTGCGCCCAGTCCATAGTTAATTTATAAATTGTTTA |
| SEQ ID NO: 375 | GCAGCAAGGCCTCCACTTCACCCCCTAAAGGTTGCCCCAAGAGCACCGTGTGACTGCTAAG |
| | GTATTTCCGGAGTCTAAAGACGATTATTCAGGTCTCATTTGCATACCCATAATACACTGCAA |
| | ACAGTATTTTTTTCGGAAAAACATTTATATATTGCTTGACATTTTTAAGTATGAGAATTTTGC |
| | ATGCAGAATTTTTTTGTATAAACTTTCTCAGGTAGTAACCCTTGGGATTAGTAGACACCATC |
| | AGTGTACTAGGAATTGCAGTTACCCGAAAATTGAGTTACAGAAGTAACTGGT |
| SEQ ID NO: 376 | GCCAGCCAGTCTTCGGCTTCGCCCCCTAACGGTGACATAAGGCACTCTGTGAAATGCTCTGT |
| | TCCGGAATCAAAAGATTGATCCGATTATTTGCATACCCATAATGCACTGCTCACAGTACAAA |
| | TTTAAAAAGGCAAAATCAAACATTTTTATTCTAAGCATATTCTGTGAAAGTTAGACTTTTGT |
| | TTAAACAATACTCTTAAAATTTTTTTCTAGGTATAGAACCTTGGCATTCACTAGTCACCATCA |
| | CTATACTAGGAGTTTCTGTTACCCGAGAAACGAGTTATGAAATTAACAAGC |
| SEQ ID NO: 377 | TGCATCATTAATGACCCAAAGACATTCTCTTTAAAAAAAAAAAATTACCACTGTCACACTCT |
| | TCAATCTTCCACACCACCTTTTTTGCGTTCCCATCTTCCAGGTCCTCTTGGTTACTAAAAATA |
| | ATGTCTACACTTCTATTTAGTTTTACTTCATCTTCTCACATCTATCAAATATTTTTTAAAGATA |
| | TGTTTAAGAATAGGAACAGTATTTTTCTTTCTTTTTTTTTAAATTGCTGCTCCTTGTAGAGCA |
| | GGGCTACGCTGCAGGCAGTGTGCCCAGAATAGCAAGAACAGTATTTCT |
| SEQ ID NO: 378 | TCTTTCTTTTTTCTTTTTTGAGACAGAGTCTCACTCTGTTGCCTAGGCTGGAGTACAGTGGCG |
| | CAATCTCAAATCTCGGTTCACTGCAACTTCTGCCTCACGGGTTCAAGTGATTCTCCTACCTCA |
| | GCCTCCTGAGTAGCTGGGATTACAGGCGCATGCCACCACACCCAGCTAATTTTTGTATTTTT |
| | AATAGAGATGGGGTTTCACCCTGTTGGTCAGGCTGGTCTTGAACTGTTGACCTCGTGATCCG |
| | CCCGCCTCGGCCTTCCAAAGTGCTGCGATTACAGGCGTGAGCCACCACAC |
| SEQ ID NO: 379 | GAAGTTCAAGAAAAGCCTGGCTGACATGGTGAAACCCCGTCTCTACTAAAAATACAAAAAC |
| | TAGCCGGGCGTGGTGGCGTGTGCCTGTAATCCCAGCTACTCGGGAGGCTAAGGCAGCAGAA |
| | TAGCTTGAACCCGGGAGGCAGAGGTTGCAGTGGGCCAAGATCGCGCCATTGCACTCCAGCC |
| | TGGGCAACAAGAGCAAAACTCCATCTCAAAAAAAAGAAGAAGAAAAATGGTTACAAAGAC |
| | TATGCAGCAATATGGAAAGTGATTATGTGTGGGCATAGTGTTACAACAACTACCTAC |
| SEQ ID NO: 380 | ACCTCATGTTGAGAACCTTTGATCCAGACTGCAGAGGGCTGGCTGGCAAATCAGCTGCATG |
| | TTGGGAGTCACCTGAGGAATGTTTTAAATGCTGATGCCTGAGTTTGGACCTGAGGTTCAAAT |
| | GTAAGTGATCTGGAATGTGGCCTGGGATCAGGATTTTTGTAAAGCACTTTGGGTGAGTCCAT |
| | GTGCAGTAGATTTTGAGAGCTGTATTCTAGTGGATCTGCCCTGTTGCTAACCCACCAGAAGG |
| | AACCAGAGCAGAACGTGCCCTTTTGGAAATAACTGGGACCATGAAGAGACTGT |
| SEQ ID NO: 381 | TTACTGTACCTTACCATAGCCTATACTGACCAATAAACTGAAAATTTCCTGTGTTAGTCCATT |
| | TGAGCTATTATAAAGGAATACCTGAGGCTGGGTAATTTATAAAGGAAAGAGGTAATTGGCT |
| | CATCGTTCTGCAGGCTGTACAAGCATGGTGCTGGCATCTGCTCAGCTTCTGTAGAGGCCTCA |
| | GGGAGCTTTTACTCATGACGGAGGCTAAGTGGGAGCAGACAGTCAGTCACATGGCAAGAAA |
| | AGTAGTGAGAGAGAGAGAGGGGAGGTGCCACAAACTTTTAAACAACGAGATCT |
| SEQ ID NO: 382 | GAAACAGTTCCAGAGTTTCTGCTGCTCTCTGTCAGAGCTCTTCATGTCCTCTTTCCAGTCCTA |
| | CGGAGCCCCACGGGGGGACAAGGAGGAGCTGACACCCCAGAAGTGCTCTGAACCCCAATC |
| | CTCAAAATGAAGATACTGACACCACCTTTGCCCTCCCCGTCACCGCGCACCCACCCTGACCC |
| | CTCCCTCAGCTGTCCTGTGCCCCGCCCTCTCCCGCACACTCAGTCCCCCTGCCTGGCGTTCCT |
| | GCCGCAGCTCTGACCTGGTGCTGTCGCCCTGGCATCTTAATAAAACCTGCTT |
| SEQ ID NO: 383 | CTACTAAAAATACAAAAATTAACCGGGCATGGTGGCGTGCACCTGTAATCCCCGCTACTAG |
| | AGGGGCTGAGGCAGGAATTGCTTGAATCCAGGAGGCGGAGGTTGTGGTGAGCAGAGATTG |
| | CGCGACTGCTCTCTAGCTTGGGCAACAAGAATGAAACTCCGTCTCAAAAAAACAAAACAAA |
| | ACAAAACAAACAAACAAGAAAAACATCAGACCTCGTGAGAACTCACTCAGTTTCACCAGA |
| | ACAGCATGGTGGAAACCACCCCCATGATCCAATCACTTCCTACCAGGTCCTTCCCTTG |
| SEQ ID NO: 384 | ATAATTACACCACTGCACTCCAGCCTGAGCAACAAGGCAAGATCCTGTCTCAAACAAAACA |
| | CCTAATTATTTGGAGTATATAGAATTTGAACTTGTTATGCATGTATGAACAGAAGTTATTTG |
| | GAGTATATAGATTTTGAAGTTGGCTTAGATGTATGATGAATTCTCAGAGGTTATGATTAATT |
| | ATCTTTACCTTTCATTCTATAAGATTTTATTGAATACCTTCTAGATGCTAGAATTTCAGCAAT |
| | GAGCAAGACTGATAAGGTCTTTGTCTAAGGAAGAACATAGACCCCATAAAAT |
| SEQ ID NO: 385 | AGAAAGAAATTGAATGCTGATTATGTGGTTTATGGTATGTGGATTTTAATAAATCAAATTTG |
| | TCACTTTAAAAAATTAGAGCTCCAGCCTGGGCAATGTGGCGAAACTGCATCTCTACTAAAA |
| | ATACAAAAAATTACCCGGGCATAGTAGTGCACACCTGTAGTCCCAGCTACTCAGGAGGGTG |
| | AGGTGGAGGATCACCTACCTGAGCCTGGGAAGTTGAGGCGGCAGTGAGCTGTGATTGCGCC |
| | ACTGGACTCCAGCCTTGTTGAGAATGAATTAATTAATTAATTAAAATTAGAGCTT |
| SEQ ID NO: 386 | TCTCCTGCCAGACTTCCCAGACCTGGCAAAGGTTTAGAAACTGTTGCTAAGAAAAGTGGTCC |
| | ATCCTGAATAAACATGTAATACTCCAGCAGGGATATGAAGCCTCTGAATTGTAGAACCTGC |
| | ATTTATTTGTGACTTTGAACTAAAGACATCCCCCATGTCCCAAAGGTGGAATACAACCAGAG |
| | GTCTCATCTCTGAACTTTCTTGCGTACTGATTACATGAGTCTTTGGAGTCGGGGATGGAGGA |
| | GGTTCTGCCCCTGTGAGGTGTTATACATGACCATCAAAGTCCTACGTCAAGCT |
| SEQ ID NO: 387 | GAGCAACTTTTCACATGCTTATTAGTCATTTGTATAGCTTCAGAGAACTGTCTATTCAGATCC |
| | TTTGTCCATTTTTAAATTGGGCTGTATTTTATCATGGAATTATTGGAGTTCTTATTTTCTGGAT |
| | GCATAAATTTTATTTTTTTAATTTTTTAAGTTGATGTAGGCGTGTTTTATAGGTGCCAGGCAC |
| | CGTACTAAGTGCCTTTTTGTACATTAACTGGCAGTCCTATGATATATGAACTAAATTTTAACT |
| | CCATTTTATACATGGCTTTTTTCTCTATTTATTTTAACATATTTATA |
| SEQ ID NO: 388 | CAATTATTCTACCATAAAGACACATATGTTAATCACAACACTATTCACAATAGCAAAGATAC |
| | ATAATCAATCTAAATGCCCATCAACAGTAGACTGAATAAAGACAATGTGGTACATATACAC |
| | CATGGAACACTATGCAGCCATAACAAAGAAGAGATCGTGTCCTTTGCAGCAACATAGTTGA |
| | AGCTGGAGGCCAATATTCTAAGCAAACTAATGCAGAAGCAGAAAGCTAAATACTGCATATT |
| | CTCACTTATAAGTGAGAACTAAACAATGAGAACACGTGGATATAAAGAGAGGAAC |
| SEQ ID NO: 389 | TCTCAGCTTGCAGTTTTTTGGACCACCCAGGGACTGTGAATACAGGCTGTAATTCCACATAG |
| | GAACCAGGTTTGGTTACAAATTCTCGGGAAAGATATCCACCAACGCCCCTACTCATTGTCAA |
| | AGTTTGAAACAGGTAATTTACCTTGCAGTCTCTACCATGGGAGTATTCATCTATGGAGTCCC |
| | AATTTTTACTGGGTGGGGTGGGGAGTGTCTCCTATTAGATACCCTGCTTTGGATGGGGTCTG |
| | GGTTTTGTCACTTGGCTCTTGTGCCTACAGGACTATGAAAAACAAAGCTCTT |
| SEQ ID NO: 390 | ATATTTCCCAAATTTGATGAAAGACATGAATATATTCATCCAAAAACTTAAACATTAAGCAG |
| | GATGAAGTCAAAGAGATCCACAGTAAGACATGTTATACTCAAATTCTACAAAGCAAGAGAA |
| | TCTAGAAAGCAAAAGGAGCTAACTCTTCACGTACAAGGAATCCTCAATAAGATTAACAGTG |
| | GATTTCTCATCAAAAACTATGGAGGCCAGAAGGAAGTGGGATGGCATATTTAAAATAGTAA |
| | AAGAAAATAACTGAGAATTCTATATTTGGAAAAACTGTCCTTCAAGAGTGGAGAA |
| SEQ ID NO: 391 | GAAGCACCTCATCAAACTGGGCGGGAGCATAGAGTTTACATGTATTTTTATTAGAACGTGC |
| | AGATCTTTATAATCTACATTACTGTTTATGATAAACATGCTATTACACAGGATCACAGATAG |
| | ATTACCCCATTTTATATGAGGTGATTATTACACACTACATGCCTGTATCAAAACATCTCATG |
| | TACCCCATATATATATATATATATATATATATATATATATATATATATATACACACATACATA |
| | CATACACACACACACACTTAATATATACGCATAAAAATTAAAATTAAAAACA |
| SEQ ID NO: 392 | GGCGTGTACCACCATGCCCGGCTAATTTTTTTCACGATGTTGGCCAGGCTGGTCTCGAACTC |
| | CTGACCTCAGGTAATCTGCCCACCTCAGCCTCCCAAAGTGCTGGGATTACAGGCATGAGCC |
| | ACCGTGCCAGGCCCAGTTCCACCATTTCTAAAAGGGAATAATAATTATACCCACCCAATATG |
| | GTTATTGTGAGGAGTAAATAAGTAGAGTGTTTAGAACTCTCTCTGGCATATAATCAGCACTC |
| | AATACGTGTTAACTATTTCACTATTTTTGTTATCATCATCATCAAATTTCAGA |
| SEQ ID NO: 393 | AACCCAGCCTGTGCCTCCACTGGCTTTGCCCACTGCACCCCCGCCTGCTCTAGCCCTGAGAA |
| | GCCTGCAGGTCTGTGCAGCAGTGGCTGCCTCTTTTAATGAAAGCATTCCCCATGGGAAGAG |
| | GCCTGGCTATTTAACAAGGAAATGTGTGGGTGGTAGAAACTGCTCCGATGCTGGCAGCGGC |
| | ATAAATCCAGCAGAAAGGAACAGGAACCTCAGAGGCTTCCTAACAGACATCATCTTCCTGC |
| | CAAGGGGAAGAAAATAATATCAGTGGGGAGAGAATGAACTCAAGACATAGGCAGC |
| SEQ ID NO: 394 | TTCCAACTTTATGCATACCTCTAACTTGAGTTTGCATCTATTACCATTTCTTGAGGCCCTAGA |
| | CTTTTCAAACTCCAGGCTTTGAGCTGAAAAAGTTGCAAAAGAATATAAAGAATGAATCTGT |
| | AAACTGATTTCTTAATTGTATATTCTGAATTCCAGACAGAGTCACTGGCCACATGGCAGTTT |
| | CAGAAATTAAACCAAAACTTAAGCTGAATCCTCTAACAAAAGTACCCATCTCCCACAATAA |
| | AAGGTAGGTAATGAATGATTAAGGGTGATAAATGTATTATGATAAAGAATTAT |
| SEQ ID NO: 395 | ACAAGAAGCTCCCTCTATGTGCCCTGTAGGGACATTTTTCTTGGAGAATCGTAACTCTAAAT |
| | TCTTACCTCGCAGAGAGGGACCCGATGGGACCTGTTTAGTAAATATTACTGAGTTCTGCCAC |
| | TTGTCGACAATCTTCCAGGGAGATAACTCCTAATTAATTAGAAAAAGTGCCTGCCCTCGAGG |
| | CGCATACATTCTATGAGTGAGAGAAACAGACACAGATGTGACATGGATGCCGCAGCGCAAG |
| | GATTTTGTCTGCATGTTCATTGCTGAGTCCCCACGTTTAGAAGGGTACCCTGC |
| SEQ ID NO: 396 | TCAGTTTCCCATTTACACCCATGATAGCACAGGTCAGTGCCCCTGAACCCCCAGTTCTTCAG |
| | TACTGCTGGGCACTGGCCTGATCTTGACTTGACTGACATCTGTGGGGAGGACAAATAAAAC |
| | TTAGATGTTGTAACTCTATCTACAAAAACTATGTTTATGTTAAAAGGAAAGGAAAGAGTATT |
| | TTATATATATACAAAATCACCTACGTAAATATGACTCAAACAGACGTACACACAGAGAAGC |
| | ATAGAAAAAAGTCTAAAAAAGTATATACCAATATGTACATCAAAAAGCCATAAG |
| SEQ ID NO: 397 | GGCAGGTGCCCATGTCTCTTCTATCTAGAGCAACAGCTTCTTGCCTGGCCTCCCTGCCTACC |
| | CATGCTCCCTCCACTCCCTGGTTTCCAAACTTCACTGCATTCCGGAATCCTCAGGGAGCTTCT |
| | CAAAACCTAATGCCCAGGCCACACTGTAGTCCAGTTGAAGGAAAGTCCCTACGGCTGCTTC |
| | CAAGCTTCAGCGCTTGCCAGAACTCCTCGGGTTATCCATTATCATGCAGCCCAGGTTGGAAC |
| | GACACCTCTGCCTGTCCCCACACTGCCCATTCTCTTCTCAAAAGCCAGGTGG |
| SEQ ID NO: 398 | ATTTCTCTAAGTGTTCAATAGAACATCTTTCTTGCCAAATAGAATTACTAAATGACAAGGAA |
| | AGAAAAAACATTTAAAATACCTGCCTGATGGCCTAATTTGTTTTAGGAAAACATTATTTGTA |
| | ACAATAAATAGGTCAAGATAAATAGAGACTAGCTTGTGATAAGAAGAAAATGGAAGGAAT |
| | CAAATGGTTCCAGAAGCAATAATAGAAAAGACATATTGCAGGAAATTGAATTATTTATACC |
| | ATATCTGTATTTTAATAAATTAGCACTAGAAGAATAATAACAATAAGTTAGTTTA |
| SEQ ID NO: 399 | CAGTCCTCCCACCTCAGCCACCTAAAGTTCTGGAATTATAGGCGTGAGCCACCGTGCCTGGC |
| | TGAATTATGAGGATCAAATGACATCATGTACACAAAGGCCTTAGAACAGCAACTTACACAT |
| | TATAGGTGCTCAGTAAATGCTCATAGTCACAGGTTAGGTGCAGTTGGGAGCTGTGGAGAAA |
| | TATAAAGTACAATCTCTTGCCTTTAAGGAGGTTGCAGTCTAGATGGGGTGAGAGGTAATAA |
| | GATAGAAGATCATGTAAATCACAATGTTGGGAATGCATGTAAAATAATACTGGCT |
| SEQ ID NO: 400 | ATCTAAATCTGTGTATATATCTACACCATCTACTCTACATTGGCATCTACGTAGTCGACAGC |
| | TACCTCTACAGCATCTACAATATCTATGTCAACTACATCGACGTCATCCACTCCATGTACAT |
| | CATCCACATCTACGGGATCTAAACTTTGCATGCACATCTATATCTAATTATATAGATGATAT |
| | AACAATCAACACATGCTTGTTATATCAAAATGAGTGGTCCTTTTAGACTAGAAAGCAAATCT |
| | AATTTTAATTTTTCCCTCCACTGGCCACACACATAAAACGTGAGTGGAACAG |
| SEQ ID NO: 401 | TTAAGAGGGACAGCGATGCTTATTTCACTGTCTATGTGTTTATACCTGATTCTTTCAACCAG |
| | GGAGACAGCCTGACCCATGGAGAAAGCACGAGTCAAAGGTCAAATGCATCCACCTGCAGG |
| | TCCTGTCACTGACCACTTAGCATCCCACAATATCACTGAACCTCAGTTTGCTCATACGTAAA |
| | ATAAAATTTTAAAAAATAGAAAATGTAGGTAGCGTGTCTAGCTCAGATGGCTCTAGTCCCTT |
| | ACTGTGACCCAACCATTTCTAATTTGGCACAAGTTACAATAAAAAACAAGCGTG |
| SEQ ID NO: 402 | AAAAAAAAAAAGCAAACTAACAAAAACCACCCTGATACGTAGAGCTGGGGAATGGCATGA |
| | AGGAAGAGTTGTCATGCATAAAAGTCCGATAACAGAAACTACCATGAAATACTCCACAAAA |
| | AACACAACCTTGCACAGAGACCATTGCAACCTTACACAAAACATTTCTGCAAGGACATCTG |
| | CCCAGCAACTGCCTGTCCAGCCTCGAACTGGTGTCACCCTAGTTGTGGATTCTTGTAGCCAA |
| | GGACAATTATTTCAAAACAATTATGTAATCCTCCTTTTTTTTCCCTTTATTTGTCT |
| SEQ ID NO: 403 | AGATTGTCTCAAGAATCCTTAAATCAGGAAGGGCCCAGAGGCCAGGGCCTTTTCCTTCAAC |
| | ACAGCTCTGTGAATCCAAGGCCTCATCCTTTAGGACCTAAGCTGGGTCTGGAATTTCCAGAC |
| | AGAATCAGGTCTCAGTTCAGACTGCCAACCCATCAGGTCAGTTTAGAACAGATGTGGCCAG |
| | AGCTGTCCCTTCCCTTGCTTCCCCCTGAGAAAGATACATGAAGCCTTGGAACTATAGCAACC |
| | ATACTGTGACCCCGACACGATGAGCATGAGGACACGTTAAGGATGGCAGAATGG |
| SEQ ID NO: 404 | TACAATCCAAGGGTTGGCTCAGGCTGTATTCTCATCTGAGCACTCTACTGGGGAGGGTTTCA |
| | GGCTTACCTGATGGTTGGCGGCATTCAGTCCTTCTAGACTGTCAGACTGAGGGCCTTGGTTT |
| | TTGCTGGTGCACTCAGTTCCTTGCTTCATATAGAGTAGCTCACAATGTAGCAGCTTGTTTCAC |
| | TAAGACAGCAAGGAAGAGTGTTTCCTAGTAAGACAGACACTGTAGTCTTACCTAATACAAT |
| | CATGAAAGTGACATACTGTCTCCTTCGCCATATTCTGTTAGAGACAGGTTAC |
| SEQ ID NO: 405 | TGGCTCAGCTTGGATCAAGTGCCCATCCTGGTCCAAACAACTGTAGCAGCACATCTGTGTAT |
| | TTGTGTATCTTGTTCCTCCATAATCTTGCAGTCCGGGGAAAGTGTGCAGATGGATGGGTCCC |
| | TGAAAAGCAAGAGTTGTTTAGACAACGCTAGAGGTGTTCAGTTCTGGGAGACAAGAGTTGG |
| | GCCAGGTTGATAACTAGCCTAAGAGAGTCGGAATCAGATGAATAAGAACTAGATCTTGGGT |
| | GACAACCTAGATCAAGAGTCTTGAGGTTCAGTCATTTCAAAAAGCTCAAGTCTC |
| SEQ ID NO: 406 | GGGATTCACCAGGGCACAAGGCGACGTGCGTCTGTCCTCACGGAGCCTAAGTGCTGGTGGA |
| | GACGGACAGTGCAGATGCCAACCGCAAATGACGAGGCACTTGCAGGTAGGGGTCAGCGCC |
| | AAGAAGGAAATGAAGGGTGCGTGTCGCTGACTGAGACTCTGGTGGGAGCTACTTAAATAGG |
| | GCCATGGGGGAGGGCCTCTCTGAGGAGGTGACATTTGAGCTGAGGCCTCACCAATCAGAAG |
| | GAAAAAGCTGTAAAGGGTCTGGGATTAACTCAACCTGGGGACTTTTAAAATAATGTA |
| SEQ ID NO: 407 | CGTATGAGAAATGAAGACTCTCGGGCGCTGCCTCTACCTACTGAATTTTAGCAAGACTACCA |
| | GGTGTTCCCCATGCACATCAAACTTTGAGAAATGCTATTCTAGGTTGCTCTCAAGTCTGGCT |
| | GCAGATTTGAAACACCTGGCAAGCTTTTAAGACACGTACCAACGCTAGGTCTGGAGGTGGA |
| | GGGGCTGTTTGTTGGTATTCATATTTTTTAGCTCCTCAAGTGACCCTCATCTAATCCTTGGAT |
| | CCCCATCCAGCCTTTCCACAAACTCTATGGGTTCTGACCTTAAAATATAGCC |
| SEQ ID NO: 408 | TCTAGCCTGGCACAGTGCAGGCACGTGCCTGTGGTCCCAGCTGCTTGAAGTGGGAGGAGAG |
| | CTTCAGCTCAGGATTTCCAGGCTATAGGGAGCTGTGGTCCCACCATTGCACCCCAGCCTGGG |
| | TGACAGAGTGAGACCCCATCTCAAAAAGAAAAGAAAAGAGGCTAGGCGCAGTGGCTCAGG |
| | CCTGTAATTCAAGCACTTTGGGAGGCTGAGGCAGGCGGATCACTTGAAGTCTGGAGTTCGA |
| | GACCAGCCTGGCCAACATGGTGAAACCCCCCCATCTCTATTAAAAATACAAAAATC |
| SEQ ID NO: 409 | AGCCTTGTCTTCTGCCACCCCACTCAGCCTTCAGATTCACGTCTCTAGATTCTTGAGGTTGGG |
| | AAATGTTTCTCTAGCAGCCATGGTTTTGCATTCATTGGCCATTCAGGTTTTCTGCTTTGCCAT |
| | CTTCCCCCGGGGGTTTCCTTATCCTCCTGGAAGCACAACTAGGCATTTAAGTCCATGTTGAC |
| | TGTATTTTACCTGCTATTTCTATGTGTTTTGCAATAAGGGCTTTTCAAGATACCCATAACTTG |
| | TGGCTAAAAATGAGGTTTGTTCCAATGTGCCTTAGAAATATCCAGTTTC |
| SEQ ID NO: 410 | CCTGACATCTCAAAAGCCTATAGAATGCCTCTAAGTTTTTCTCTGGAGGATCAACTGAGAGA |
| | TGCATTCCTCCGTGAAGGCTTATATCTCATTGGCTGAGGGCTACTCTGCAGGGGTTACCTAT |
| | GTTATCCACCCCCACTCCACTCCTGCTACAATATGCATTAATTAGTGCCAATTCTTGGGCATT |
| | CTTGGCCACAGAGTGAGGGAAAGAAATCTGTTTTAGGAGAGATACTGTCAGCTTACAGTGA |
| | GTTGAAACCCACACAAAAATATCTACTGCACTTGTTGCCGAATTCAATGGGG |
| SEQ ID NO: 411 | GTTCTCTGATGCTGATGGAATCAAATTAGAGTTAAATACCAGGGAAATCATACAAAATTTTC |
| | ACACTCAGAAATTAAGCAAGATACTTCTAAAAATACAGAGATTAAAAAAGTCTTACAAAGA |
| | ACAAAAAATATGTTCGGGTAAATGAAAATAAAAAGAGAATATACCAAAATTTCTGGGATTC |
| | AGGTAAAGCAGTGCTGAGAAGGAAATTTATAGCACTAAACACTTACATCACAAAGGTGGAA |
| | AAATCATGAATCAACCATCTAAGCTTGCAGTTTAAGAAACTAAGGAAAAAAATAT |
| SEQ ID NO: 412 | TGAATCCATTATGGAAGTAGTATAGACTATTTTAGAATGATCCTTAGCTATATTACTGTTATT |
| | TCCTATTTTAAGCTTTGAGAAAAACTTGTTTTCAACTCTCAAGCAGTTCATGCCAGTTCCTGG |
| | AATAAAAAGAACATCTGGGAAAAATCCCAATTAGTGTTGTTTTGTCCTAGTTCTCTTATTTC |
| | CAGTTTTTCCTTAGAGATACAAACGAAAGACCCCTAATTATAGATTTTAAAGTTTTCCTTAT |
| | CCTCTTCCATTTGGCTACTAGGTGATTCTACTGTGTAAAAACTTAGCTCC |
| SEQ ID NO: 413 | GTAATCAAATATGGGCTATACTGCTTCTCCTGCAAATAGGTGATCAGAACATGGACAGGAA |
| | AGACACACCAATTTCTATTCCTCTGAGAAGGGTGCTGGGGATAGATCTGGGGCCTAAACTG |
| | TGTTCATGTGTTTTCTTAACTAAAACAATTTTTTTAAAAAAAAGAATCTGAAGCCATTATAG |
| | TAGAATAATGTTAATATGTATTCACTCTGGGTGGTGGGTACATGGATGTTTGTACTTTGAAA |
| | TATCTTCGCATTTTTAAATATTATTCTGTGAAAAGAAGGGGAAGACAAAATGTA |
| SEQ ID NO: 414 | TCCGCGGACCAACTCTCGCGACAGCCAGCTCAAAGCAGGCAAGAACCGGAAGGGGCGGGG |
| | ACGTTCCCCGTGAGCCTTCGCGGTGCTGGCTGCTCATCTGCATACGGAAGTTCGGCACATTA |
| | TGAATTATTTATTTTCCTCGAGGGAAAAAATTAAATGAAAAGCAACAAAATACATTATTAA |
| | CAAGTGAGACAAACTTCAATGGAACTGGATCATGACCTCAACAGTCAACTACGATAGTCAT |
| | CATACGCCTAATGAGAATAGAATTCATTACCTAGGAAATAAACTAAAAACGTCCTT |
| SEQ ID NO: 415 | CAGACACAAACAGACAAAATTGCTAAGTTTTAAATGGCTCCTGAGAAGTGGGAATTGGGGA |
| | TGTGAAATGGCCTTTGCAAAAAGTGTAACAGTGACAAAGTTATGGCAGTGTGTAGATCTGA |
| | CCTAACTGACTCCATCTTGCTTCTGTGTTCATTCCTGGGCATAGGCCAAACTAACTTTGGGA |
| | AGAACTTTAACTTTGACATAAAGATTGTAACAGCCCTTTCCTGAAACAAACCCCATTCTTGC |
| | ATGGGAACCACACTGCCTTTGTAGGACTAACACATGAGCCACAATATTATGGTT |
| SEQ ID NO: 416 | CCTGACAATTTCACAACCTAGGAAGGATGTTGAGCAACTGGAATTTTTATATGTTGCTTCTT |
| | AAAACACAAACTGGACCTTTCTTTCAATGTTAAATACATAGTTTACCCAAAGGTGGAAAAC |
| | CTTATCTTCACACAAGAATCTGTACAGGAGTGTTTATGGTAGCTGTATTCATAATTGCTTAA |
| | AACTGGAAGCAAACCAAATGACCTTCAGTGGGAAAATGGATAAACAATATGTGATAGAAG |
| | TATCACAGATGTTTACATAACCTGTGATACTTAGCAGTAAAAAGTAAACTAATAA |
| SEQ ID NO: 417 | AAGATTACCAAATGAGTATTGAATGGCTTCAAAGCTGTTTCCCGGGCACCCCCAAACCCTA |
| | GATTCCCTTGAATCTTTCCTGATTCAGCCTGGTCCCTGGTAGGGAAGGACCCCAGCCTTTAG |
| | GAAATATGGGAGAACTGGAGAAATAAAGCAAAGGGGAGGGCGGTGGCAGTGTGGTAAGAA |
| | ATAAGTCGAAGTAAATTGGGGACACTTAAGCTTTCAGTTCCTTTAAACATCTGTTTCCTTTG |
| | GGCAATTCACACTTCCAGAAGAGTTCTTGAGATGCCGTTTAAAGATAAAAAAAAG |
| SEQ ID NO: 418 | TTAAATCCGGGAGGCAAAGGTTGCAGTGAACCGAGATCACGCCACTGCACTCCAGACTGGA |
| | CGACAGAGCGAAACTCCATCTTAAAAAACAAAACAGAACAAAAAAGTGAGAACATACGAT |
| | GTTTGCTTTTCCATTCCTGAGTTACTTCATTTAGAATATAATGGTTTAAAAAAAAAAGAATA |
| | TGATGGTTTCTTTCCTTCCTCCCTTTCTTCCTCCTCCTCTTCTTCTTTCTCTCTCTCTCTCTCGC |
| | TCTTTCTCTCTCAGTAAAATAACCTAATTTATTTTTTTAAATTCCCAGTACC |
| SEQ ID NO: 419 | AATCCCAGCAATTTGGGAGGCCGAGGCAGGTGGATTGCCTGAGCTCAGGGGTTTGAGACCA |
| | CCCTGGGTAACACGGTGAAACCTCCTCTCTACTAAAATACAAAAAATTAGCCGGGCGTGGT |
| | GGCGGGCACCTGTAAGTCCCCTACTTGGGAGGCTGAGGCAGGAGAATCATTGGAACCCAGA |
| | AGGCAGAGGTTGCAGTGAGCCAAGATCGCGCCACTGCACTCCAGCCTGGGCAACAGAGGG |
| | AAACCCTGTCTCCAAAAAAAAAAAAAAAAAAAGAATATTCTTGAAAAAAAAGAAATA |
| SEQ ID NO: 420 | AGTTCCCTGGAGAAGCCTCTCCTTCTTCCAAGTCCCAGCCTGAGGCCTCTCCTCTTCTAGGTG |
| | AACAACACCAATCTGCAGGATGTGAGGCACGAGGAAGCTGTGGCCTCACTGAAGAACACAT |
| | CTGATATGGTGTATTTGAAGGTGGCCAAGCCAGGCAGCCTCCACCTCAACGACATGTACGC |
| | TCCCCCTGACTACGCCAGCAGTACGTACTCATCAGCCCCTGTCCCTGGTCTAAAGCTCTGTC |
| | CCCTGGTTTCTGGAGGGGAAGAAGTTCATACCCTTTGTTAAGAGAGGGCAAGC |
| SEQ ID NO: 421 | ATCCTTAGGGTATTTAGTCAGAATGGAATATTATGAGTCAAAGATATTGATCAGAAGTCAGT |
| | CGAAATTTAAAATATCTTCTCTTGGATTTATTTTCTTCATAACTATTATCATGCAATAATGAT |
| | GCAAATGCAGTACAAACAAAAACATAGATGAACACAGATGCATTTGCTTGATAAGTACAGA |
| | CAAGGAAACAGCATGGTAGAAATTTGCCCAACTAGACTTTCATTCTAGTAGCTAAGCTTTAT |
| | GCTTTTTTTCTTAACTTGATATTTTAAAAACTTGAAAAAAAAAAGAATCAGT |
| SEQ ID NO: 422 | TGAAGTGTGAGTTTGTGTTTGTGCGTGTGCTGTGACTCTTTGATGTGTGGTGGAAGATTTCTG |
| | GGAACTAATTTTAGTTCCTGAGTTCTAATTCTAGTTGAGTCTGAGATCACCACAGGTAGTGT |
| | CACTATTTTGAGCAATTGTACAATTTCTTTACATTATCCCCAAAAGGCCTATGCCCACATAG |
| | GTACACAGGAGTGCTAATCAATGTAGTGAGTAGGTAAAAACCCTGTTTTTGTCTTTTGTAAG |
| | TTTAAACTGTTTTCTGTCCCCTAAAGAGAAACCCTTAAATAAAAATGATGA |
| SEQ ID NO: 423 | TAGCTTGAACCCAGGAGGTGGAGGTTGCAGTGAGCTGAGATTGCACCACTGCACTCCAGAC |
| | TGGGCAACAGAGCAAGACTCTGTCTCAAAAAAAAAAAAAAGAAAAGAAAGAAAAGGAAA |
| | AAAAAGAAAAACAAAAGTTGGTTGCTTTCTGTAAATGTGACCAGATTTGAATGAATGCATG |
| | TAAATCTATGCTGTACTTTGCTGGGGAGGTTTTTGTCAGGGATAAGGATGGGGGCAAACTAT |
| | GTTAAGGGAAGTGATGACAAGAAAGCCTTCCCTGCGATTAAGAAATTATAATAATAT |
| SEQ ID NO: 424 | GAGACATTGGACCAGCACCTTTTGTTCAAGGTGACAGTAACATAAGCCTGCTCAAATAAGT |
| | AACATTTCCCTTTGCCACTGGCTCATTTGAGTGGAATGACAAAGTTTGCTTGCGTTTGTGTG |
| | GACAGACCCACACAACCCTGGCACTGATAAATATTCAGTATGTCTTTATGTTTTCACACTAA |
| | GCAGCACTGAAAAGTGGTCAACTTTTTGTTTGTTTTTTGTTTTAGAGAGACATCTGTAATCCT |
| | GCTGATGCTCATGTGCTGAAAACTATGACCGTACTCAATAAAGAAGGAGACA |
| SEQ ID NO: 425 | CCTTAGGGTATTTAGTCAGAATGGAATATTATGAGTCAAAGATATTGATCAGAAGTCAGTC |
| | AAAATTTAAAATATCTTCTCTTGGATTTATTTTCTTCAAAACTATTATCATGCAATAATGATG |
| | CAAATGAAGTACAGACAAAAACATAGATGAACACAGATGCATTTGCTTGATAAGTACAGAC |
| | AAGGAAACAGCATGGTAGAAATTTGCCCAACTAGACTTTCATTCTAGTAGCTAAGCTTTATA |
| | CTTTTTTTCTTAACTTGATATTTTAAAAACTTGAAAAAAAGAAAAGAATCAGC |
| SEQ ID NO: 426 | TCCAGAAGACCGAGAATGGCTGTTAGATACAATAGAATGCTCTAAGAGTCACTATCAAAAA |
| | TGAGCATATCAGTGCATAAAATGTGTGGTAGTTTAGCAGTTATTTCATTGTTTACCGTAGTG |
| | TTTTTTCTTACAATTTTGTAGAAGCCTGTGTCAGAATTAAGAACTTTTTTAGAAGAGAATAAT |
| | CATGGATGATTGAAATTAACATTTTAAGCTGATACTGAAAATTATTCTAAATTCTATTACAT |
| | TTCTATTTGTATTTTCTTTCAAAGGCTAATGGAAGTCTTAAAAAGAAAATGG |
| SEQ ID NO: 427 | ATAAAATTCCAGAAGACCGATGATGGCTGTTAGATATAACAGAACGCTCTGAGAGTCACTA |
| | TCAGAAATGAGCATATCAGTGCATAAAATGTATGGTAGCTTTATTTAGCAGCTGTTTCATTG |
| | TTTACCATAGCATTTTTTCTTACAATTTTGTAGAAGCCTGTGTCAGAATCAAGAAGTTTTTTT |
| | TAGAAGATGATAATCATGGATGATTGAAATCATACTGAAAATTATTCTAAATTCTATTATAT |
| | TTATAGTTGTATTTTCTTTCAAAGGATAATGGAAGTCTTAAAAAGAAAATGG |
| SEQ ID NO: 428 | CAGAGCGAAAATGACTGTTAGATACAATAGAACACTCTAAGAGTCATTATCAAAAATGAGC |
| | ATATCAGTGTGTAAAATGTATGGTAGTTTTATTTAACGGTTGTTTCATTGTTTACCGTAGTGT |
| | TTTTTCTTACAATTTTGTGGAAGCCTGTGTCAGAGTTAAGAACTTTTATAGAAGAGGATAAT |
| | CATGGATGATTGAAATTGACATTTTAAGCTGATACTGAAAGTTATTCTAACTTCTATTACATT |
| | TATAGTTGTATTTTCTTTCAAAGGATAATGGAAGTCTTAAAAAGAAAATGG |
| SEQ ID NO: 429 | GAAGGCTGGGAAAGGCTGTTAGATACAATAGGGTGCCATAAGAGTCACTATCAAAAATGA |
| | GCATATCAGTGCATAAAATGTATGGTAGCTTTATTTAGCAGTTATTCCATTGTTTACCATAGT |
| | GTTTTTTCTTACAATTTTGTAGAAGCCTGTGTCAGAATTTGAACTTTTTTAGAAGACAGTAAT |
| | CATGGATGATTGAAATTAACATTTTAATCTGATACTGGAAATTATTCTAAATTCTATTACATT |
| | TATATTTGTATTTTCTTTTGAAAGCTAACGGAAATCTTAAAAAGAAAATGG |
| SEQ ID NO: 430 | AACTCACCGAGCCTGGGGAATACGACCCACTTATAATGTCCAAGACCTTCCTCTCATCCCAA |
| | GTTTATGACAATTTCCACCCATAATCAAAATTTCCTAAACTTACTTTCTGTTTTTTTGTTTTTT |
| | TTCATTTGGTAGAGACAGAGTCTCCTTCTGTTGCCCAGGCTGATATCAAACTCCTGAGCTCA |
| | AGGGATCCTCCCACCTCAGCCTTCCAAAGTGCTGGCATTTCAGGCATAAGCTACCGGGCCTA |
| | GCCTTACTTCACTTTTCTAAGAAACAGTATTATTACCTCATTAGTCAATA |
| SEQ ID NO: 431 | GCAACAGAAAACCTTTTTTTGAGGAGTCCCTAAGGCTTCACCAGTTTGTCAAAATTGGAGCT |
| | TGATTGCTGAGACTTTATTTGCATACCCACAATGCATTGTGCACCAAATAATTTTCGTCTTTA |
| | AAATTACTAGTCCCCTTTTTCCTTCAATCTATCTTTCCTGAAACTGAGATTGCTGTTCCAGAA |
| | TTGTCTGAAGAAAAAGGCAGAAGTCAATAGCTCTTTTGGGCAGAAAGAAATTTACCATTAG |
| | CCTGTTGGGAGTAGCCATTACCTGAGAACTGAGTGCCACTCATCGAAGTTC |
| SEQ ID NO: 432 | GAGCTTGCACCACTGCACTCCAGCCTGGGAAACAGAGCAAGACCCTGTCTTAAAAAACAGA |
| | TAAACAAGCAAACAAACTTAGACAAAGAGTTACCAAATGATCCAGAAATTCTACCCCCCAA |
| | AATATACCCAAGAAAATAAAAATATATATCCACGCAAAAACTTGTACATAAATGTTCATAG |
| | CAGCATTAGTCGTAATAGCCAAAAGTAGAAACAAGTATCCATCAACTGATGAATGGATAAA |
| | TAAAATGTGGTATATCCATATAAAAGAATATTATTTGGCAACCGAAAAAGAAGTAT |
| SEQ ID NO: 433 | GATTGGTCGGTTGAGTGGCAGAAAGGCAGACGGGGACTGGGCAAGGCACTGTCGGTGACA |
| | TCACGGACAGGGCGACTTCTATGTAGATGAGGCAGCGCAGAGGCTGCTGCTTCGCCACTTG |
| | CTGCTTCGCCACGAAGGGAGTTCCCGTGCCCTGGGAGCGGGTTCAGGACCGCTGATCGGAA |
| | GTGAGAATCCCAGCTGTGTGTCAGGGCTGGAAAGGGCTCGGGAGTGCGCGGGGCAAGTGA |
| | CCGTGTGTGTAAAGAGTGAGGCGTATGAGGCTGTGTCGGGGCAGAGGCACAACGTTTC |
| SEQ ID NO: 434 | CGGAAGAACCCCGAGTCCACTGTAAGCTCAGGGGAGAGCGGGAGCCAGGGAGGTGAAGTG |
| | CGCAGACTCGGCAGAGGCGGCGGGCAGAACCGCGCGGGGGTGAGAGGGCGCGGTGGCTGC |
| | GGGGCGGGAGCCGCTGCTGAGAGGCGGCCTGGGTTGTCTTGTGGGGTGACTGTCGGTGGAA |
| | TCTTTGGTGGAGAGTGGTTTGGAAGAATGGCGAGGGGCGGCAGTGGGGAGGGTGGTGACCC |
| | TGAGCGACCGGCCAGGGCGAGGAGGCTGTGCTGTCCCTGCAAGCCATGTGCTCATTTC |
| SEQ ID NO: 435 | TGTTAGTTACCTTGCTGTTCCTTGATAAAAAGCTACCTTCTTACATATCTTACTTCCAATAAA |
| | GAGGCAGATTCCCTATGTACTGTTTATCTCGGGAAAGAAATACATGTGGGAAAAGAAGCAA |
| | TTTTCTTCTCATTGCAATTAAATTAAATCATGACCACCTGTTGTGGGATTATCCTGCCATCTG |
| | CTGTTCAAAAGAGAAACAACAGCAGAGAACCAGAAAGAAGAGGCAAAATGTTGGAAAGCT |
| | TTGCTCCCAGATGTCCATCAGTTGATTACGGTTATTGGTATAGTTAAAATTTT |
| SEQ ID NO: 436 | GGTGGATCACCTGAAGTCAGGAGGTCGAGACCAGCCTGGCCAACATGGTAAAACCCTGTCT |
| | CTACTAAAAATACAAAAATTAGCCAGGCATAGTGGTACGCACCTGTAATCCCTGCTACTTA |
| | GGGAGCTGAGGCAGAAGAATCACTTGAACCTGGGAGATGGAGATTGCAGTGAGCCAAGAT |
| | CACACCACTGCACTAGCCTGGGCAACAGAGCAAGACTCTGTCTCAAAAAAAAAAAAAGTGT |
| | GTGTGTGTGTGTGTGTATATATATATATATAAACATAAACTCACTAACATAAGTTTA |
| SEQ ID NO: 437 | CCAAATATGACATAAATACTTGATAAATTTTCTGGTAAATATATTTTAATGGAATTTTTTTAA |
| | CTGAGGAGACATTTGAAAGTTACTTGAATAAGTTCCAATGAATAAATACATGGACAGAATT |
| | TTAATCTCTGGAATTTGTGAGATGGGGTTTTTACGAGTCTTTGACAAATTTTCCTATGTTTAA |
| | GACTTTTCCTAAGTATTTGTATGTATGTTGCTTCATGTGAAACAACCTTTTTAAATTTGAAAT |
| | TAATAAAATGGGTTTTTCAATCAACTCTGAGTGAAGATAGAAAAATTTAT |
| SEQ ID NO: 438 | AGTCAAGTGCCTCTCTCCATTTACTGGTAAGAGAGAGAGGGTTTAGAGGAACTCTTGTTCCG |
| | GCGCTCAGCTCATTTGCATCCCAGAATGCATTGTAGATACGAGAATTATTACCAGGGTTATC |
| | TGTTTGAATAATAATATTTAAACTTTTTTTCTTTGTCAGGAGATTTTACCCAGTGAGAACATG |
| | TTTAGGACACTTTTCTACAGTGGAAGAAAAGCTTCTGTCTGCAGGTCCAAAGGCACCGTAA |
| | GTAGAGGGAGACCAGTCAATAGCTGGGAAGCCAGGCAAAAGGCTAACAGGCA |
| SEQ ID NO: 439 | GCCCCCTGGTGGTGGCCTAACGCCTAGAAAAAGTAACGAACTATTTAAGGTATTTCTGGAA |
| | ACCGAGGGGGTGGTGACTCAGCGTCATTTACATACCCACAATGCACTGCACACAGACCACG |
| | TCCTACTTTAAAAGGAGATTCTCCCTTTTTTCCCCCTCTTTTCCTTTACCATAATAACGAAGC |
| | TCCTTTCACACAATCGTCTGAGACAAAAACAGAAGTCACTCTTTTGGGTTAATAGTAACCAT |
| | TGCTAATCTAGTAGTGACCGTCCCCCGAGGACTGTGTGCAACCATTCCAACAC |
| SEQ ID NO: 440 | CAGCAGAGAGGAGCATAGGAGAGCAAAGGAGATCAGTGACCCATGGCTTCCCCGGTGGCG |
| | CGGAACAGCCCGGAGCCGCCTGTGATTTGCATACCCATGGTGCACCACGAAAAGATACCCT |
| | CAAGATGCTTGCACTCCCTCTGTGCGCGCATTTCTGCACTGTTTTAGAGCATGATGCCTCTTA |
| | CACGCATCTGTGTGCATAAACTACATATAGGGAGTGCGTACCACGCAGGCATCCAACAACC |
| | ATAAGTGTGTTAAGTGTTAGTTCTCCCTGCGAGGTTCGAAGCGGAAGTCACGAAT |
| SEQ ID NO: 441 | CCTGTGAGGGTGGAGTGTAAGGAGACCATCAAACGACCTGAGAGAATGTGCTCAGCCATCC |
| | ACAAAGTGCTATTGAAAGCAGGGGGCTTGAGGAAGAATGTGGACCCTGGAGTTCTAAGACT |
| | ACTGTGACAGTTTTATTCCCCAGCCATGCCTGGCCCAGAGTCTGGCCTGGGGGTGTGGCTGG |
| | TGCCTCTGCACTACTCTTGGGCCTTCATGGATTCTGCCAGCAGGTGTCCTCAAGGTGCCCCC |
| | AAATGAATTCGCTGCCATTGGGCAAGATCAGGCACTCAGTAAAAATTGAAAAAC |
| SEQ ID NO: 442 | TAATTCAAGTCCTTTACCAATTGTGTGGTTTTGTGGTTTAAAACATGGCTTACCCTTATCTAT |
| | CACAACAATTTTCTAATTTCTGAAAAGTCCCTCCTTCTCTGTCCCAATTCCATACTTTTCTGT |
| | TCCACATGAAGTTGAGATACCCAAACTTGTTTAAAATAAATTTAAAGTATCTGGAAAATTAA |
| | TTTAACAATCCTTTCAGTAATTTATTCTGTACATTCACAACCAAGTACTTTTCAGAATGGTTA |
| | TAAAACCTCAGGAATATATGAAATTTATGAAATAAAAAAAGAAAACTTC |
| SEQ ID NO: 443 | AAAATATTCATTAAACCATACCATAAACAGATGTGCTATCATCCAGGCTTTGCTGTTCCATT |
| | TCTAGAGCACAGGCAGAGTAGATTTAGCATAATTCTGAAGGGCTCTAGGATTTTCAGAATG |
| | GCAAATGAGCACTGATTTCAATTTAAAGTCACCAGCTGCATTAGCCTCTAACAAGAGAGTC |
| | AGCCTGTCCTTTGAAGCTGTGAAGCCAGGCATGGACTTTTCCTCTTTAGCTATGAAAATCCT |
| | AGATGACATCTTCTTTCAATAGAAGACTGCTTTGCCTACCCTGAAAATCTTTGT |
| SEQ ID NO: 444 | CATAATATTTATGTCTGCAAAGGTTATGAGGACAAAATATGCTAATATGAGTCAAGCATTTA |
| | GAATGCCAGCTGGCCCTATAAATGTTAGCTACTATTATGGTGCTTTAGATGAATTCAGATAT |
| | CACTTTAAGTTCTAATGTTCCATGATTCTGGAATTAATAAAATACTGTGCAAACAACAGAGA |
| | ACCATCTTTACTATGAAATTAACCCAGTTTGTGTCTTTGCCTTAAACAATTCTAAAGTGATTC |
| | TCAAAGTTCGTAAGCCAAGAGAGTGGCTTTTAGTTTTTAAAGGACTAAATC |
| SEQ ID NO: 445 | CTGCCTCATCCTCCCTAGTAGCTGGGATTACAGGCGTGTGCCACCATGCCCAACTAATTTTT |
| | TGTATTTTTAGTAGAGACAGGGTTTCACCATGTTGGCCAGACTACTCTCAAACTCCTCACCT |
| | CAGGTGATCTGCCCGCCTCAGCCCCGCAAAGTACTGGGATTACAGGTGTGAGCTACCGCAT |
| | CCAGCCAGAAAGGTAGATTCTAATCTGATCTGTGTCTTCTAATGCCCACTGTTCTCCCATTG |
| | CTTAATATCACCGTATGTTAGGCTGTTCTTGCATTGCTATAAAGAAATATCTA |
| SEQ ID NO: 446 | TCAGCTTCATTCTCAGACATGCTCTCCCTCACCATGGTGGCAGAGGTAGCTATCAGTACTGG |
| | GTTTCTGTCATTCTCACAGCTATGGGTGCCTCTTTTGCCATACTTCTAGCAGTAGTGAGTACC |
| | AAGGAGGGACCTTGACCAGCCCAGATTGGATTGCCTGCCTGAATCAGCATGGCCAGAAATA |
| | TGCAGTAGACAATAAGCAGCATCACTTTGGCTAAGGCCTGTTCATGTGTCCCATGAGAGAG |
| | GGTAAATCCCACCAGAATTACACAGAACGGGTTCCCCATGGAAAAAGGAGGGG |
| SEQ ID NO: 447 | CAAAGTAGGAATCAGAAACCGTTTCCTGATGCTTTGCTTCAGCTTCCCAACTGCTTTGAGTT |
| | TTAAAGCTCTTGTTGCTGTCAGGAGTATACACTTTTCTAAGTAGAAGAGACCTGATGTCAGA |
| | GACACTGATTTATAATAAAAATAATTTTTACTTTGTGTTACAGCATATATAAGAACACATAA |
| | GAAGTGCTCCCAGTGGTTATTGATTTCTAAGTGGAATTAGGTTATAAATAATACAATGCAAA |
| | ACTAGATTTGTTAATGTTAAGGTATAGTTGTTTAACAAAATTATGATTTTCC |
| SEQ ID NO: 448 | GATTGTGCCACTGCACTCTAGCCTGGGTGACAAAGCAAAACCCTGTCTCAAAAAAAAAAAA |
| | GTTATCTCCTAAAATTTTGTAAAATTACTTTTTTTTCTCCAGGGGAGGAGAAAACATCAAGA |
| | AAACAAAAAGAGAAATATCAAAATCATTCTTTCGGTTCTTTTGTGGTTTTCAACCACTTTTG |
| | GGTTTTCCCCTTGCAGAAACCACAGAAATATTCTCTTAGAATAAAATAGTTTATCTGTTTAA |
| | AAAAAGAAAGAAAGAAAAGAAAAGAAAAGAAAAATGTGATAAATGTGATCTCT |
| SEQ ID NO: 449 | GACTCAAGTTTTTTTGGGAGGGAAACTGCCTTGCTTTCCTCTTCTATATATTACAGGGTCTAG |
| | CAAATTAAATTACACATGGCAGGCAATGAGTAAATATTTACAAAATCCTTTTTGAATAAGA |
| | AGATGTAAAGGAAATGTTCTCAGTTTTAAAGATTATTATGAGGTACTATGGCAAAATGGAA |
| | AAATATAGGCTCTGGATTGGACAGACCCAGTTTTGAATTTCAGTCACTTAAAAATCTCTTTG |
| | AACCACATCCTCTCCCTAAAGTGGGATAGTATGTACTCCTTAAAAATTGTTGG |
| SEQ ID NO: 450 | CCCACCTACCTGCAGGCCTCACCCCATGGGGGATGGCCTGACTTGGTCTCATTATTTGTTCC |
| | TCTATCCCCCAGGCCTCTTCCTTGGTGCTGGCTAAGGGCTAGGCTGGATGTTTAGTTGTAGC |
| | CCTAAGGAAAAATTTTAGTATGTCCACTTTTATACACAGAGGCACAGATGGTAAGCAGTTAT |
| | GAAAGTTGTCCGAATCAAAATGGAGTAATTTATGTTAAAACCCTGGCAAATGGAGCCAGGG |
| | AAGGCCATCAAGGGAGAGTTCTTACACATGAATGCCTGATAAGAACTGTCACA |
| SEQ ID NO: 451 | TCGGCTGAGTCGGCGGAGGGTGGGGCGGAAGAGCAGACGGGGACTGGGAAAGGCGCTGTC |
| | AGTGACATCACGGATAGGGCGATTTCTATGTAGATGAGGCAGCGCAGGGGCTGCTGCTTCG |
| | CCACTAAGGAGTTCCCGTGCCGTGGGAGTGGGTTCAGGACCGCTGGTCGGACCTGAGAGTC |
| | CCAGCTGTGTGTCAGGGCTAGGAGGGCTGGGGGGGGCGGGGAGGTGCGCGGGGCAAGTGA |
| | CCGTGCGTGTAAAGGGTGAAGCGTGTGAGGCTGTGGCGGGGCGGAGGTGCAAAAGCTT |
| SEQ ID NO: 452 | GGGTTCGGAAGAACCCCGCGTCCACTGTAAGCTCAGGGGAGAGCGGGAGCCAGGGAGGTG |
| | AAGTGCACAGACTGGACAGAGGCGGCGGGCAGAACCGCGGGGGTGAGAGGGCGCGTGGTT |
| | GCGGGGGGGGAGCCGCTGCTGAAAGGCGGCCTGGGTTGTCGTGTGGGGTGACTGTCGGTGG |
| | AATCTTTGGCAGAGAGTGGTTTGGAAGAATGGCGAGGGGGGCAGTGGGTAGGGTGGTGAC |
| | CCTGAGCGTCCGGCCAGGGCGAGGACGCTGTGCTGTCCCTGTAGGGCATGCGCTCATTC |
| SEQ ID NO: 453 | TTCCCAAGAAGGGTTGGGGACGAACCCCCTGTCCACTGTAAGCTCAGAGGGGAGCCGGGGC |
| | GAGGGAGGTGAAGTGCACAGACTGGGCAGAGGCGGTGGGTAGAAGCGCTGGGGTGAGAGG |
| | GCGCGGTGGCTGCGGGGGGGGATCCGCTGCTGAAAGGACGGCTGGGTTGTCTTGTAGGTGA |
| | CTGTCCGTGGAATCTTTGGCGGAGAGTGGTTTGGAAGAATGGCGCCGGCCAAGCAGAGGGG |
| | AAGGTGGTGACCCTGAGCCTGCGGCTACGGGACAGGAGGCTGTACTGTCCCTCCTCT |
| SEQ ID NO: 454 | CGGGCGGAAGAGCAGATGGGGACCGGGAAAGGCGCTGTCGGTGACATCACGGATAGGGCG |
| | ATTCCTACGTAGATGAGGCAGCTTAGGGGCTGCTGCTTCCCACGAAGGATTTCTCCTGCTTT |
| | GGGAGCAAGTCCAGGACCGCTGGTTGGACGTGAGAGTCCCAGCTGTGTGTCAGGGCTAGGA |
| | GGGCTCCGGGTGGCATGGGGGGGGGTGGGGGTGGGGGTGCGGGTGCGCGGGGCAAGTGAC |
| | CGTGTCTGTAAAGGTTGAGGCGTATGGAGCTGTCGCAGGGCGGAGATGTGTGAACTC |
| SEQ ID NO: 455 | GTGGCAGAGGGTGGGGCGGAAGAACAGAAGGGGACTGGGAAAGGCACTGTCGGTGACATC |
| | ACCGATAGGGCATTTCTGTGTAGATGAGGCAGCACAGGGTTGCTGCTTCGCCAGGGAGAAT |
| | TCCCCGTGCTGTAGGAGCAAGTCCAGGACCGCTGGTGGATGTGAGAGTCCCAGCTGTGTGT |
| | TAGGGCTAGAAGGGCTTGGGGTGGTTGGGGATAGGCGGGGGTGGTTCTCAGGGCAAGTAAC |
| | CGTGGGTGTAAAGGGTGAGGCATATGGAGCTGTGGCAGGGCGGAGGTATGTGGACTG |
| SEQ ID NO: 456 | AGAAAGGCAGACGGGGACTGGGAAAGGCACTGTCGGTGACATCACGGATAGGGCGACTTC |
| | TATGTAGATGAGGCAGCGCAGGGGCTGCTGCTTCCCCACCTGCTGCTGCGCCACGAAGGAT |
| | TTCCCGTGCCGTGGGAGCGGGTTCAGGACCGCTGGTCGGACCTGAGAGTCCCAGCTGTGTG |
| | TCAGGGCTAGGAGGGCTGGGGGTGGGGGGGGGGGGGGGGGGGGCTGCGCGGGGCAAGTG |
| | ACCGTGCGTGTAAAGGGTGAAGCGTGTGAGGCTGTGGCGGGGCGGAGGTGCAAGATCTC |
| SEQ ID NO: 457 | GGGTTTGGAAGAACCCCGCGTCCACTGTAAGCTCAGGGGAGAGCGGGAGCCAGGGAGGTG |
| | AAGTGCACAGACTGGACAGAGGCGGCGGGCAGAACCGCGGGGGTGAGAGGGCGCGTGGTT |
| | GCGGGGCGGGAGCCACTGCTGAAAGGCGGCCTGGGTTGTCGTGTGGGGTGACTGTCGGTGG |
| | AATCTTTGGCAGAGAGTGGTTTGGAAGAATGGCGAAGGGGGCAGTGGGTAGGGTGGTGAA |
| | CCTGAGCGTCCGACCAGGGCGAGGACGCTGTGCTGTCCCTGCAGGGCATGCGCTCATTC |
| SEQ ID NO: 458 | GCAGAGGGTGGGGCGGAAGAGCAGACGGGGACGGGAAAGGCGCTGTCGGTGACATCACAG |
| | ATAGGGCGATTCCTATGCAGAGGAGGCAGCTCAGGGGCTGCTGCTTCACCACGAAAGATTT |
| | CTCGTGCTGTGGGAGCTAGTCCAGGACCTCCGGTTGGACGTGATAGTCCCAGCTGTGTGTCA |
| | GGGCTAGGAAGACTTGAGGCGGCATGGGGGGGGGTGGGGGAATGCGCGGGCCAAGTGAC |
| | CATGCGTGTAAGGGGTGAGGCGTATGGAGCTGTGGCAGGGCGGAGGGGCGTTCATTC |
| SEQ ID NO: 459 | CGGGTTGGTCGGCTGAGTTGGCGGAGGTTGGGGTGGAAGAGCAGACGGGGACTGGGAAAG |
| | GCACTGTCGGTGACATCACGGATAGGGCGACTTCTATGTAGATGAGGCAGCGCAGAGGCTG |
| | CGCTTCGCCACATGCTGCTTCGCCACGAAGGAGTTCCCGTGCCGTGGGAGCGGGTTCAGGA |
| | CCGCTGGTCGGACCTGAGAGTCCCAGCTGTGTGTCAGGGCTAGGAGGGCTCGCGGGTGCGC |
| | GGGGAAGTGACCGTGCGTGTAAAGGGTGAGGCGTACGGGGCGGAGGTGCAGGAGCTC |
| SEQ ID NO: 460 | TAGGCTGAGCGGCAGAAAGGCAGACGGGGACTGGGAAAGGCACTGTCGGTGACATCACGG |
| | ATAGGGCGACTTCTATGTAGATGAGGCAGCGCAGGGGCTGCTGCTTCCCCACCTGCTGCTTC |
| | GCCACGAAGGATTTCCCGTGCCGTGGGAGCGGATTCAGGACCGCTGGTCGGACCTGAGAGT |
| | CCCAGCTGTGTGTCAGGGCTAGGAGGGCTGGGGGGGGTGGGGCTGCGCGGGGCAAGTGAC |
| | CGTGCGTGTAAAGGGTGAAGCGTGTGAGGCTGTGGCGGGGCGGAGGTGCAAGAGCTC |
| SEQ ID NO: 461 | GCCGAACGGCCTTTCCCCCGTCCTGCCCCTCGTCCACTGTAAGCTCAGGGGGGAGCGGGAC |
| | CCAGGGAGGTGAAGTGCACAGACTCGGCAGAGGCGGCGGGCAGAACCGCGGGGGTGAGAG |
| | GGCGTGGTGGCTGTGGGGGGGGAGCCGCTGCTGAAAGGAGGCCTGGGTTGTTGGGAGGGTG |
| | ACTGTCCGTGGAATCTTTGGCGGAGGGTGGTTTGGAAGAATGGCGAGGGGAGAGCAGAGG |
| | AGAAGGTGGTGACCCTGATCGTCCGCCAGGGGAGAGTAGGCTGTGCTGTCCCTCCTCT |
| SEQ ID NO: 462 | GCTGAGTGGCGGAGGGTGGGGCAGAAAAGCAGACCGGGACGAGGAAGGCGCTGTCGGTGA |
| | CATCACGGATAGGGCGACTTCTATGTAGATGAGGCAGCGCAGGGGCTGCTGCTTCGCCACT |
| | GGCTGTTTCACCACGAAGGAGCTCCCGTGCCGTGGGAGCGGGTTCAGGACCGCTGGTCGGA |
| | CCTGAGGGTCCCAGCTGTGTGTCAGGGCTAGGAAGGCTCGGGGGTGCGCGGGGCAAGTGAC |
| | CATGTGTGTAAAGGGTGAGGTATATGGGGCTGTGACAGGGCAGAAGTGTGTGAAGTC |
| SEQ ID NO: 463 | GCTGAGCGGCAGAAAGGCAGACGGGGACTGGGAAAGGCGCTGTCGGTGACATCACGGATA |
| | GGGCGACTTCTATGTAGATGAGCCAGCGCAGGGGCTGCTGCTTCGCCACATGCTGCTTCGCC |
| | ACGAAGGAGTTCCCGTGCCGTGGGAGCGGGTTCAGGACCGCTGGTCGGACCTGAGAGTCCC |
| | AGCTGTGTGTCAGGGCTAGGAGGGCTGGGGGGGAGGGGGGTGTGCGCGGGGCAAGTGACC |
| | GTGCGTGTAAAGGGTGAAGCGTGTGAGGCTGCCGGCGGGGCGGAGGTGCAATAACTC |
| SEQ ID NO: 464 | CAGAGATATAGAACAGACACTAAAAGGACAGTCCATAAAAGGACAAATAGATAATTTGGA |
| | CTTCATCAAGAATAAAAGGTTTTGCTCTGTGAATGACCTTGTTAAGAAGAGCAAAAGACAA |
| | GCCACAGACTGGGAGAAGATATTTGCAGATCATGTATCCACCAAAGGAGCACTACCTAAAC |
| | ATGTAAAGAACTCTCAAAAATCAACAATAAAAAACAAACAGGCTGAGCAGGAAACTGGAA |
| | AGAGACATGAAGAGACATTTCACTGAGGAGGATCCATAGAAGGAAAACAAGCACATAT |
| SEQ ID NO: 465 | GCTGAGTGGCGGAGGGTGGGATGGAAAAGCTGACTGGGACGGGGAAGGCGCTGTCAGTGA |
| | CATCAGGGATAGGGCGACTTCTATGTAGATGAGGCAGCGCAGGGGCTGCTGCTTCGTCACC |
| | GGCGGCTTCGCCACGAAGGAGTTCCCGTGCTGTGGGAGGGAGTCCAGGACCGCTGGTCGGA |
| | CCTGAGAGTCCTAGCTGTGTATCAGGGCTAGGAGGCCTCGAGGGTGCGCGGGGCAAGTGAC |
| | CGTGCGTGTAAAGGGTGAGGCGTATGAGGCTGTGGCGGGGCGGAAGCGTGCAGACTC |
| SEQ ID NO: 466 | GCCGAATGGCCTTCCCCCCGTCCTGCCCCTCGTCCACTGTAAGCTCAGGGGGGAGCGGGAC |
| | CCAGGGAGGTGAAGTGCACAGACTCGGCAGAGGCGGCGGGCAGAACCGCGGGGGTGAGAG |
| | GGCGCGGTGGCTGTGGGGGGGGAGCCGCTGCTGAAAGGAGGCCTGGGTTGTTGGGAGGGT |
| | GACTGTCCGTGGAATCTTTGGCGGAGGGTGGTTTGGAAGAATGGCGAGGGGAGAGCAGAG |
| | GAGAAGGTGGTGACCCTGATCGTCGGCCAGGGGAGAGTAGGCTGTGCTGTCCCTCCTCT |
| SEQ ID NO: 467 | GCTGAGTGGCGGAGGGTGGGGCAGAAAAGCAGACCGGGACGAGGAAGGCGCTGTCGGTGA |
| | CATCACGGATAGGGCGACTTCTATGTAGATGAGGCAGCGCAGGGGCTGCTGCTTCGCCACT |
| | GGCTGTTTCACCACGAAGGAGCTCCCGTGCCGTGGGAGCGGGTTCAGGACCGCTGGTCGGA |
| | CCTGAGGGTCCCAGCTGTGTGTCAGGGCTAGGAAGGCTCGGGGGTGCGCGGGGCAAGTGAC |
| | CATGTGTGTAAAGGGTGAGGTATATGGAGCTGTGACAGGGCAGAAGTGTGTGAAGTC |
| SEQ ID NO: 468 | GCTGAGTGGCAGAAGGTGGGGCGGAAAAGCAGAGCCGGACGGGAAAGGCGCTGTCAGTGA |
| | CATCACGGATAGGGCGACTTCTATGTAGATGAGGCAGCACAGGGGCTGCTGCTTCGCCACC |
| | GGCTGCTTCGCTACGAAGGAGTTCCCGTGCTGTGGGAGCAAGTCCAGGACCGCTGTTCAGA |
| | CCTGAAAGTCCCAGCTGTGTGTCAAGGCTGGAAGGGCTCCGGGGTGCGCGGGGCAAGTGAC |
| | CGTGTGTAAAAAGGGTGAGGCGTGTGGAGCAGTAGCAAGACGGAAACGTGTGAACTC |
| SEQ ID NO: 469 | CAGAGGGTGGGGCAGAAGAGCAGAGCCGGACCGGGAAAGGTGCTGTCGGTGACATCACGG |
| | ATAGGGCGATTCCTGTGTAGATGAGGCATCTCAGGGGCTGCTGCTTCGCCACGGAGGATTTC |
| | TCCTGCTGTGGGAGCAAGTCCAGGACCGCCGGTTGGACGTGATAGTCCCAGCTGTGTGTCA |
| | GGGCTAGGAGGGCTGGGGGTGGGGGTGGGGGGCGGTGGGGGGTTGCGCGTGGGAAGTGAC |
| | CGTGCGTGTAAGGGGTGAGGCGTATGGAGCTGTGGCAGGGCGGAGGCGTATGATCTC |
| SEQ ID NO: 470 | ACTATACAAATCTAATTTAATAATCTCCAGAACATTAAGAAAGGTATTGTCGGTGACATCAT |
| | CGATAGGGCATTTCTATGTAGATGAGGTAGCACAGGGCTGCTTCTTCACCATGGAGGATTTC |
| | CCGTGCTGTAGGAGCAAGTCCAGGACTGCTGGGTGGACGTGAGAGTCCCAGCTGTGTGTTA |
| | GGGCTAGGAGGGCTCAGGGTGGTTGGGGATTGGTGGGGGTGGTTCTCAGAGCAAGTGACCA |
| | TGCGTGTAAAGGTGAGGCGTATGGAGCTGTGGTGGGGCAGAGGTATGTGGACTG |
| SEQ ID NO: 471 | GGTTCAGGCAGCGGGTTGGTCGGCTGACTGGCAGAAAGGCAGATGGGGCCTGGGAAAGGC |
| | ACTGTCGGTGACATCACGGATAGGGCGACTTCTATGTAGATGAGGCAGCGCAGGAGCTGCT |
| | GCTTCCCCACCTGCTGCTTCGCCTCGAAGGAGTTCCCGAGCCTTGGGAGCGGGCTCAGGACC |
| | GCTGGTCCGGCCGGAGAGTCCCAGCTGTGTGTCAGGGCTAGGAGGGCTGGGGGGTGCGCGG |
| | GGCAAGCGACCGTGCGTGTAAAGGGTGAGGCGTACGGGGCGGAGGTGCAGGAGCTC |
| SEQ ID NO: 472 | TACCAAAGATGATGAAAATAAGTATATGTACAAAATATTTTAGTATTTATGTGCCTGTAAAT |
| | ACAAAAGGAGCAATAAAAGTGATTTCATTTCAGAAGGTGAACATTTTGAAAGAAATAATAT |
| | TCATGTAAATTCTGAACTAAAATAGAATGAAATAAAATTCTGAAATAAGATAAAAATAGAA |
| | TGTTAGCATTATAGGAAACTATAGAGATTATTTGAGCTAATCTTCTCATTTTATGTATATGGA |
| | AGCTGAGAAGTGACATATCCATAGTCATACAGCTAATAAATAATCAGGATGGA |
| SEQ ID NO: 473 | GGGTTTGGAAGAACCCCGCGTCCACTGTAAGCTCAGGGGAGAGCGGGAGCCAGGGAGGTG |
| | AAGTGCACAGACTGGACAGAGGCGGCGGGCAGAACCGCGGGGGTGAGAGGGCGCGTGGTT |
| | GCGGGGGGGGAGCCACTGCTGAAAGGCGGCCTGCGTTGTCGTGTGGGGTGACTGTCGGTGG |
| | AATCTTTGGCAGAGAGTGGTTTGGAAGAATGGCGAAGGGGGCAGTGGGTAGGGTGGTGAC |
| | CCTGAGCGTCCGACCAGGGCGAGGACGCTGTGCTGTCCCTGCAGGGCATGCGCTCATTC |
| SEQ ID NO: 474 | GGGCGCGGTGGCTCACGCCTGTAATCCCTGCACTTTGGGAGGCCGAGGTGGGCGGATCACG |
| | AGGTCAGGAGATCAAGACCATCCTGGCTAACATGGTGAAACCCCGTCTCTACTAAAAAATA |
| | TGACAAAAATTAGCCGGGCGTGCTGTCGGGCGCCTGTAGTCTCAGCTACTCTGGAGGCTGA |
| | GGCAGGAGAATGGCGTGAACCCCGGAGGCGGAGCTTGTAGTAAGCCGAGATCTTGCCACTG |
| | CACTGCAGCCTGGGCGACAGAGCAAGACTCCGTCTCAAAAAAAAAAATTTTTTTTA |
| SEQ ID NO: 475 | ATTTGTCAGATTAGATTATAAAAGCAATATCTAACTGTATACTGCCTAAAAGAAATCCTTTT |
| | AAATATAGACAAAAATAGGTTAAAGCTAAAGGGATAGAAAAAATGTACCATGCTAATGCT |
| | AATCAAAATAAAGTTGGAATCGCTATACTAATACTAATAATGTAGATTTCAGAGCAAAGAA |
| | TATTGCCCGGGATAAAAAAGAATCATTTCATAATGATAAAGAGGTCAATTCATCAAGAAGA |
| | CATAATAACTCTAAACACTTATGTACCCAATAACAGAGATTCAAAGTACGTGAAGC |
| SEQ ID NO: 476 | AATCTTACCACATTCTCATAGTAAACGCACAGGAAAGATCCCCTGTGGCTCTAGCAGGATG |
| | GAGGGAAGAGGGGAGACTAATCATATAATCCAGAACATTCTCCAGAAAAAGGCCTAGTCTC |
| | CAGGGAAAAAGACTTCCAGAGACTTATCCTATCCATGACAACTCCAGTCCCATTCACTCTTG |
| | CTAGCTCATGTAAGGGTGGAAAAAAGCTAAGAGACATTTGTGAAAGTCATGGTCCAGGGAC |
| | ATCATTATACAAAAGACTGAAATTTAATTATAAGATTATAGAAATTCTTCTTCCC |
| SEQ ID NO: 477 | GTGTCTCTTATTCCTCCATTAGTACAACTAATCTAGAGTTGACTATATATTGAAATACTTTGT |
| | ACTACTTAAAACTACCAGTGCTCAACTTCTGTGCCAAAATTTCTGCTATCTGGAGCTAAGTT |
| | GGGCCTAACTGTAGCCGTTCTCATGGCTGAAACTAAATTGTCATCCTTTGCTTAAATCTCTGT |
| | GGCTCCGGTGTATCCACATATGATGGAAAAAGATTTTGCCCTGAATTAGTATTTATTTCCTA |
| | AAAGAAGCCGTAAAAGGAAGTAATGATCAAGTCTTTTTAAAATGGAATAT |
| SEQ ID NO: 478 | ATGGCCTCTACTTCGCCCCCTAGTGGTAGCACCAAAGGCACCCGGTGAATAACAAGGTGTG |
| | GAATCTCAAGGTGATGACTCAACATCTCATTTGCATACCCATAGTACCCTGCACATAGTAAA |
| | TTATTTAAAGCTTAAAAGAATATTTCTTTTGACTCAACATTTCTAACTATGGGAAGATTAAA |
| | GAATTCTTTTTGTGCAAACTGTAGGCAGTAATTCTTTTTGTGCGAACTGTAGGCACCATCGG |
| | CGTACTAGGAGTTGCGGTTACTGCAACAATGAGTTGAACTAATTTGTAGCATT |
| SEQ ID NO: 479 | GCAGTATGTAGCATTTGATTGGCTCTTTTCACAGAAATATGCATTTAAGGTTTCTCCATGTCT |
| | TTTTATGGCTTGATAGCTCATTTATTTTTAGCACTGAATAATATTCCATTGTGTGGATGTACA |
| | ACAGTTTATTCATTCACCTACTAAAGGGCACTTGGCTGCTTCTAAGTTTTGGCAATGATGAA |
| | TAAAGCTGCTATAAACATCCATGTGTAGGTTTTTGTGTAGATAGGAGTTTTCAGCTCATTTA |
| | GGTAAATACCAAGGAGTATAACCGCTTGATTGGATGGTAAGAATATGTTT |
| SEQ ID NO: 480 | CATCCTCCTGTCACCTACGAAAGTTCTATCTTACCACCTTTGAATTACAGTTTCACAAACAA |
| | CTACAGTTTTCTCCAAAAGTATTCTGATAATTGAAATGTTCTGTCTTATTAAAATAGCATGA |
| | AGCATGTTCTTGTAAGTTTTGGAGACTGAAAAACAGAATCAGTTATCTGTCTTCAGCAGCAA |
| | AATGTCAAATAAATTAAATTGATAAACATCATTGACTTTTACAAAGCCCACCTGTACAATGC |
| | TGTGTAATGTCTGGAACTCCAGTGATATTCTTTCCTTTAAAAGTTGGAGCCT |
| SEQ ID NO: 481 | AAACAATTCTTTCTTCCACCAATAGATATTTCCCCTTGAGGCCAGTAACCTGATTATGTATTC |
| | CTTTTGGACCCCATAATAAGCAGGACCTACTTCACCAGTTCAGCTCAGTATATGTCTCATCT |
| | CCATGGGGCACTCCCCAGTGGGCAATTTGGTCTCACAAGTGAGTTCTTGGCCATCCCCAACA |
| | GGAGATGAATTCTCAGGGGAGCTCAGCGGGATGCCATCGAATCTTTGTCTGATCAATTTCCA |
| | TTGTTCTGATTTTACCCACCTCAACAGCCTTCACATCCTGAGCAAGCTGGC |
| SEQ ID NO: 482 | CAGAACAGCGACACGTCTGTTTTATGTTTTATTCAGATCACTGTGACTACACGTTGGGAACA |
| | TAGTTACAGAGGCAAAGACCAGGAAGGATGTCATTGCAATCATCTAGATGACAAATAAAGG |
| | CAGCTTGGACCAGGCCAGTAGCCATGGGAGGAGGAGCACTGGAGATAGATGATATAGATCT |
| | AGGTGTAGATATAGGTAGATATCGATGATAGATATCGATGATATAGATAGATATAGATGAT |
| | AGAGATAAATCTATATGTGGGTGTATTTTTAAAATTGTGATAAAGTCCATATAAC |
| SEQ ID NO: 483 | ACAGTGCATTAATAATTAATTAGTGCTTAATCAGGAGCTCTACATAGCAGTTCATTACCCAA |
| | GGACACTCCCCATTATAACTGTAGCAAATGGGTCTGGGCAGGCAGCCCTGCCAGCCTCTTTT |
| | GAGGCTCACCAGTGACTTTGAGGCTCCCCTTCCAGGGCTGAGGTCACTAACCTCCCCTAGAC |
| | CTGGCGACCAGTAGCTGGTGTAAGAGGTCCCCAAGAACAGGGGTGATCTGACTCACCCTGG |
| | AGAAATTTAAGACACGCTGGGAAGGCAGAGCCCTTGCTTAAAAATAACTCCTC |
| SEQ ID NO: 484 | CTGACCAGGCGCAGTAGCTCATGCCTGTAATCTCGGCACCTTGGGAGGCCGAGGCAGGCGG |
| | ATCACATGAGGTCAGGAGTTCGAGACCAGCCTGGCCAATGTGGTGAAACCCCGTCTCTACT |
| | AAAAATACAAAATTAGCTGGGCAATGGTGATGTGCACCTGTAGTCCCAGCTACTCGGGAGG |
| | CTGAAGCAGGAGAATCGCTTGAGCCCAAGAGGTGGAGGTTGCAGTGAGCCGAGATTGCGCC |
| | ACTGCACTCCAGCCTGGGCAACAGAGCAAGACTGTCTCAAAAAAAAAAGTAAAAGG |
| SEQ ID NO: 485 | CACCCTCTTCCACTGAGTGGCCCTTTCAACCCTTACAAAGCCGGAGAGCTCAAGTTCTGGGC |
| | CCTGTGGCCTTGGTGAGCAACACCCTGGACCGATCAGAACGACCCAGTGAGTTGGGAAAAC |
| | GCTCCTTTTACCGATAAACTTGAAGCAACTCATGACTGTGGCTCTGGCACCACCTCAGCGGA |
| | GGCGGAAGTGCCATAGAACTTTTAAAAAATATATTTACCAAGAATTAAAACCAGGTGAAAT |
| | AGTCTTGTATTTGGTAGTTTAAGGGAAAGCAATTGGCTGGATTAAAATGTATTC |
| SEQ ID NO: 486 | TCAATGAACCACATAAAATTGTGATACATAACAAATATTACTCAAAAAAAAGGCCAAAATA |
| | GTATGATGTGGGATTTAAAAAATGTATTCTAATTTTTAGGAGTATAACTAGCTTAATCCAAG |
| | TAATTAATACAACCACTAATAAACGAATTTCTAAGCCAAGACACTGCTATCCAAAAAAGTA |
| | GATAAATGTACTATACACTTTAGCAAAGTACACATTTGTGTTAAGGCTCCCATCTTCTACAT |
| | TCTAAAAAAGAAAAAAAATTATTCACGAGGAGAATTTTAAAAGTGGAGAGTTCA |
| SEQ ID NO: 487 | CAATTATTTGGACAATGATATAGAAATGATCCTTATCAAATTTCTCAGTTGATGAGCTAGGA |
| | GAAACAATGAACATGTTAGATAACTGAATCAAGATCCAAAAACACTTTGACAAACTAAATA |
| | TAGCAGACTGCTGAGGAAATGAGTGGTGATACACTGGGTGCTTTTTTTGTTCAGTAAGAGCT |
| | GGAGGCAAAAGGTCACAGGTCGATAGATGCCGTCATGAGGAAGGGTCCTATGGATTACCAG |
| | TAGCTTTGGTGATGGCCAGATCAAATGGAATTGCCTGAAGCATTAAAAAAAAAA |
| SEQ ID NO: 488 | TCTTCTTTCTACTTTCCATTCACGTGGGCATCTTCCTTCCCAGGGTTTCGAAAGCCCCTCACG |
| | TCCTCCCTGTCATTTCATAATCCTGCTCACAGAGCACTTTTTCCATCTTCCTCAAACAGTCCA |
| | ATCTTGGCCCGGGAGAAGGGTTCAGGGACACAGGCTTCCTGTGGGCAGAAATTTTATTCTA |
| | AAATTTCTACAATAAACATGTTTATTTTTACAATCACAATAAATTTTAAAACTTTAAACAAG |
| | GAAATGTGTAAAGAAAAGGGAAAATGGGAGGGGTACACAAAGTTTATGCTC |
| SEQ ID NO: 489 | AGCTTTCAAACCAGCTTCCCCAGGGCGCTGTGCAGGGCCCGGCCCCTTGGTGGCCACGCCTC |
| | CCGGGCGCAGAGCGGGTGGGACATGCAAATGAACGGCCCTCGCGACCAAGGAAAAGGGGC |
| | GACAAAGCTCGAAGTCACTAAGAGTCAAGGAGCGGACAAAATGTCTCAAAGGGCCTATGTT |
| | TTCAATATAATATTTACATCAGAAAGCAGGTGAGATGTTTACCGAAGGTGAGGTTTCTCACC |
| | GTGACCTTAAGGTGGATGAAAGAAGGGAGGACGTGGGAGGCCCCGGGTAACTGGT |
| SEQ ID NO: 490 | GTTGCAAGGATTTAATATAACTTATGTAAAGTGGGCAGAAGTGCTCCGCAGGCACAGAGCA |
| | GGTGCTCAATAAATGCCAGGACCAAAATACCACGAAACTGTAATTTTTATAAGGAAAAGGA |
| | TCTTTTCTTCAGTGCTCAACGAGGAAATATTATGGGGCCGTGCGCAATAGCTGACGCCTGTA |
| | ATCCCAACGCTTTGGAAAGACTAAGCGGGAGGATCGCTTAAGCTCAGGAGTTCGAGGCTAG |
| | CCCGGGGAACATAGCGAGATCCCTTCTCTACAAAAAAAATTTTTTTTAATTAGCT |
| SEQ ID NO: 491 | CCAGTGTGTTAAGAACTGCAAGGACCACTGGAAATACAAACACAGTTAAGACAGTAACGCA |
| | CTTTGAATTAAAAGGCAAGGAGATGTTATTACAAATGAACATCTAAATTATTGATATCTCTA |
| | TAAAATTGAGGTTGTAGTACGATGTTCAGTCTTGGCCTGTGTACGTTTTGGACCAGATAATT |
| | GGTTGTAGGGTAGAAGAAAGAGCGTCCTGTGCATTACCGGGTGTTTAGCAGCAGGCCGGAC |
| | TCTACACACTTAATGTTAGTGGCCCCCTGATTTGCGACAAACAAAAATGCCTCT |
| SEQ ID NO: 492 | TTCAGATTTTGGAATACTTGCATTATGCTTACCAGTTGAGGGTCTCAAATTTGAAAATCCAA |
| | AGTCTGAAATGCTCCAGTGAGCATTTCCTTTAAGCATCAGGTGGGAGCTCAAAAAGTTTCAG |
| | ATTTTGCAGCATTTCAGATTTTCAGATTTGGTCTGATCGACCTGTACTAATAGCAGTCAAAA |
| | TGAAATGTAAGGCAAAAAACATAACTAAACATAAAAAGGGCCATTTCACAAAATGTTCATT |
| | TCCCCAGCAAGATAATAATTTTACATTTCTATGCATTAACATATTATAGAAAG |
| SEQ ID NO: 493 | GTCACTAACCAATGCTTTTACGAATACAGTAAAATAAATTACTAGAAAAATGAAGTATTAA |
| | AAAAGATCAAGAAAGTAAGATCAATGTTAATTTTCACTCTAAGAATCACTAGATTTTATCAA |
| | ATAGCTATGAAAGTTTCTAAACACTCTCCATTTCCATACTTAACACAGTCTGGAAGCAAAAG |
| | TTTCAGAGGATGATACAAGGTCACAGACTACATTTTAAGTAGATTTGGTCTAAAAGACCACT |
| | TCTAATTTTGAGGACACAGACTTTTAGTACAGCTCTTTAAGAATAGTTATTTG |
| SEQ ID NO: 494 | AAAGAAGTTTAATTGACTCACAGTTCCACATGGCTGGGGAGGCCTCACAATCATGATGGAA |
| | GACAAAGGAGGAGCAAAGTCACGTTTTACATGGTGGCAGGCAAGGGAGCTTGTGTAGGAA |
| | CTCCCTTTTATAAAACCATAAAATCTCATGAGACTTACTCACTATCACAAGAACAGCATGGG |
| | AAAGACCCACCCCCATGATTCAGTTACCTCCCACTGGGTCCCTCTCACAACACATGGGAATT |
| | ATGGGAGCTACAATTCAAGATGAGATTTGGGTGCAGACACAGCCAAATCATATCA |
| SEQ ID NO: 495 | TTGACATGATTCCCAGAGTTGTCCCTTGACTCCCAAAGGGGTTCAGAAAATATGGGAGCTG |
| | GAAGGATTGAGCTGACCACTTAGGTTTATGAGTGTGAATACACAAAATAGATGTTTGGGTC |
| | AGGCCTCAGGAATAAGATACTGAGACTGATTATGTTCAGTGAGAAAGATGTTTCCTTGGGA |
| | GCATTACATCAGGTCAGAGCAGTATCAGGGAAAGTTTGAGAGCACTGGAGAGCTGTAGGGA |
| | GCTGAGGTGTCAGCCTCCACCTCAACTGAGAAATAGGAGAGGTGTGGCGTAAGTAG |
| SEQ ID NO: 496 | GCACATGTATCCCGGAACTTAAAGTATATTAATAATAAAAAAAGAGATTAAGTAAAAAAAA |
| | AAAAAAAAAAAGAAAAGAAAAGGAGAGGTAGGTGAGAGAGAGAGAGAGGAAAGAAAGCT |
| | AGTATTTGTAGTTATCCTATTCTAAAAAACTACTATTCAACTAAGACAACTAAGAAAAATAT |
| | ATTCCAATAAAAAATTTTAAAATTACATTATGAGGGTGAACATGACTATTTAAACAATCTGT |
| | ACTTTAATTAATTAATTAAGAACCCAGATTAGTAAAAAAAATTTTTAAATCCAGAT |
| SEQ ID NO: 497 | CCCACAGCAGTAAAGAATAATTACGCAGTTATTCTTCTGGTTATGTTTTAATTTCAAAAAGT |
| | TAAGTGTGATTTTCCTTTTTGCTGGGATTTCTGTCTTGAGCAAATAATTATTCTTATGAAAGT |
| | ATCAATTGCAGTTACTGGTTAAAAATGTAAGACCTAGGAAATTTAAGTGTTGTTTCTATTTT |
| | AAGGTATATACATAATTTATAACCATTTACTTCTAGAGAAAATCCTGAATGGTTAGTAATAT |
| | CAAAACATTTTCAACAAGAAAACTAAAATGAAAAGAAAGTTTAAAAATTAC |
| SEQ ID NO: 498 | AGTATGTTCTGATGAACATAATCTCTAGAAAGAATACTACAGATCTTCAAGCATAAGATAG |
| | AACTGTATGTATTGACCTGGAAAGCCATGATAAATATGAGGAAACAAATCTGAAAGAATAA |
| | ATTCCAAATTGTTAGCAGTGTTTACTAGGAGGAGATTTGGGAGCACTCTGCCTATAGTCTCC |
| | AGAAAACCAGCAAACAAGTTCCTGCCAAAAGCTATATAATTTCTCGAAGTTTAATTAGGTTT |
| | TAATTAAATATACTTAAATCTTTGTAATCATTTCTCTACCTGAAGAATTACTGT |
| SEQ ID NO: 499 | CAGCACGCAGCCAGGGCTCAAACCCCTGCCCTCCACTGCCTCCTTCACAGGTGTTCCTTTGG |
| | GAACCTCCTTGATTCACCGCTCAGTTTACATGGCCAGCTTTATTTGTCCCTGGATGTCACCTG |
| | ACACCCGTCCATGAACACTTTTCATTTGCAGTCAGCATGCGTGGCCAGCACATTCCAGCCCC |
| | TCCTACCCTGTCTCCTATTTTGTGCTATTTCCCAAAGGCCTTCTGTTTCCTGTGGTGCAGAGG |
| | CCAGCAGCAGCCCGGGAAAACGCACCTGCAGGTGACTGTAAAGAGTTCAG |
| SEQ ID NO: 500 | CCCAGCACTTTTTGGGTATCTCTCCTATGTCATAGGAGAGACATTACATCTATGTAATAGAT |
| | GTAATGATGTTGTACAAGGCTGAGCAACCTCAGCCTTGCTCTAAAATTATTTACATCATAGT |
| | TTACCATTCTTTGAGGGTGGCACTTTGCATTTCATAGAATAATTGAACATTTAGAGAATTAA |
| | TTGAATGAATGCATGGATGCAGAGTTGGATGGAGGAATGGATACATGATGGTAGATGAATG |
| | AATGGATGGATGAAGGGACGATTGGTCCAGGAGGAAATAGAAAAATAATTGCA |
| SEQ ID NO: 501 | CAAAGACACCCCAGGGGACTCTAGCTCCTGTGGTTGAATCACCCCTGCTGAGGCCCCAAAT |
| | ATCATGGAACACTGAGTCTGAATAACCCACAGAATCCATGAGCAAAATAAAATGATTATTT |
| | CACAGCCCCAAGTTTTGGAGTCATTTGTTACAGAGCTAGAGTAACTGGGACATCATCTCTCA |
| | GTAAACAGAATCACGAATCTGAATGAGAACCCCAAGTGAAAAACCAGCCAGGGGTTTAAA |
| | CACACTGGGAACAATGTGGTCCCTGAAGCAGAAGATGAAGTTAAAATCCCAGCTCT |
| SEQ ID NO: 502 | TAGCTTGATTTGAAATTGGAACAGGCTGAAAATAAGAAGAAAAGCCAAAATATATGTCTCT |
| | GGATTTTTTTTATCTTGTTCAATTAATTTATTTCAAATGAAGAAAGCCAGTTTCCATCCAGTT |
| | ACAATCATGAATTCCTCAGAGTTTATTGTTAGGTCAATATTTAGAAACAGCATTACATTTTC |
| | TCACAGAAACAATGAACAATAATGATTATGTTTGCTTCCAGGCTAGCTCACAGAAGCCTAT |
| | ATTCCTCTAATTGTAATGAAAACACATCTTTGTATTGAAAATAGTAGAAAATT |
| SEQ ID NO: 503 | GCAAGCTTCCAGGGCCTGCCAAGTGCCTTTTGCAGGGTCAGTTCTCCTGAGGGCTGGTAGGG |
| | TGGGGCTGAGTGGATAGAGCTGAGACTGCAAGAGAGAAGACAGGAAGTGCTGGGCACAGG |
| | CTGTGGTCAGAGGCCGGCGATCAGGCAAGGGACTCTGAGGTGTCCACAAATTCTCAGTTTG |
| | AGCCTGGCCCCAAAGGTTGTGATGAAGGCACATATTTGGTCAAGTTAGGAGGATAAAATCT |
| | ATTTTTGTTTTGCAGTTTGCATTTTCAGCTTGATTTCTAGCTGAAATATCTAGACA |
| SEQ ID NO: 504 | TCACTCTGGGCAAGTGGACAATGACACACACCTCCTGGGTTGTTCTGCAGGGGTAAAATGA |
| | AAAGATGCCTGTGACACAGGCATTATTTTTCCACTGTATACAGACCAGGGGATTATCACATT |
| | GTGGTGAGATTTCTTTTTATTTTCTTTTAATCCATGACTGAAAAGTGGTATGGAATTAAGAA |
| | GGAAAGGTAAAAGCAAAAAACATAAAATTTAAAAAATAATAGTCGTCATTATTAACGAGG |
| | ACTTCTAAAGCAAGTTTCTAGCCTTTTTCCTTTTTCTAGATCAGAATTTGCTCCT |
| SEQ ID NO: 505 | TTGAGAAAATCTTAACCTATTTCATGGAGACATACTTTCCAGAATTAACAAGATGACCAATT |
| | AAAAACAAATAGCCCTCCTTTAGTCCATGGTTGCCTTAAACAATATATTATGTATTAATTTC |
| | TTTCTAATGACTGTGCATAGCTTATTGTGTGGTATATGGGCTTTGTTATATTTATTACTGTCA |
| | TTATTTTCCTTATTTTCCTATCAAAACTAGCCTTTCAGTGAATATCTTTCTAACATACATCAC |
| | TTTATACTAGTCAGATAAATATAAAACTCAGGTGAAGAGTGGACAGTATG |
| SEQ ID NO: 506 | TTTTGGTTATTAGAATATTTTACCATATTCTTACATAAAGTAAGTAGAGAAAAGGAAATGTT |
| | ATTAAGAAAAACCATAAGAAAGATAATATATATATATATATTTATTATTCACTAAGTGGAA |
| | GTTGATCATCATGAGGATCTTCATCCTCATTGTCTTCACATTGAGTAGGCTGAGGAGGAGGA |
| | AGAAGAGCAGGGGTTAGTCTTATTGTCTCAGGGGTCATGGTTACCCTGCAAGTTTCTTCAAA |
| | CTGTTGTAAATCTCCAAAAAAAATTGGTATATTTATTTTAAAAAATCCATGCA |
| SEQ ID NO: 507 | TGCACTCCAGCCAGGGTGATAGAGCGAGACTCAGTCTCAATAAATAAATAAATAAATAAAT |
| | AAATAAATAAATAAATAAATGGTTTCAATTAAGTAGCTATAATGAATATTATTCTTAAACCA |
| | TGCCATTCTGGAAAAAGATTGAAACAATGGAACAGGGTCATTCAGGAAGCTTCTGTGATAC |
| | CCCTCTATAGACTCATAGATCAAAAAAAAAAAAAAAAAAACCACAAGACCCTTTTCCTTTT |
| | CTGAATAAAGGCCACTCAACGCTGGTATTCTGGGAATTGTCCAGATGAGGATGCA |
| SEQ ID NO: 508 | CTCTAGCTGGGAGGCAGCCATAAGTGGTCTACCTTGAATCCTGCTCCAGTGCCAACAGGCTG |
| | CTGGGCTTTTCTGGAATAAATTGGAGATCCCCTCCCCACCTGATCATGGGGTGGCATTAGGG |
| | TCCTGGAGAAGGGCTATAGAGAAGCCCAGCCTGGTCCCAGGTCCCACACAGAGCTTCTCTG |
| | ACCCAGCACCCATGATTGGGAAGGTCTCCATGTTCTGAACTGGGGGTCCATATTCTGAAAG |
| | GTGGGCATGCGCGGCATCACCATTGCTGGGGCTGCCGTCAAAACACATAGTGCT |
| SEQ ID NO: 509 | GCGTGTGTCCCCCCGTGCATGCGGGGAAGGAGCAGTGGGGGAAGGCTGAGTTGGGCTTTCA |
| | ATTAAAAAAAATGTGCATACACTTAGTTTTTTGATGCAAAATTGCCAAAGGCGTGTACCGTG |
| | AAAACCTACCTCGACACCTGCCCCCTGCCTCCTGTTCCCTTTCCGGAAGCGCCTGCCTTACA |
| | CCGGTGTGTTCCTCTAGAGATGGCCAGGACCTGGGTGAGGCAGCACACACTCACAGGACCT |
| | TCCCAACACTCATTATGAAACATATCACATGGAAAACTGCAAGAATTACAGGCA |
| SEQ ID NO: 510 | GCCAGGTGCAGTGGCTCACACCTGTAATCCCAGCACTTTGGGAGGCAAAATGGGTGGATTA |
| | CCTGAGGTCAGGAGTTTGAGACCAGCCTGGCCAACGTGGCGAAACCCCATCTCTACTAAAA |
| | AAACAGAAATTAGTTGGATGTGGTGGCACACACCTGTAATCCCAGCTACTTGGGAGGCTGA |
| | GGCACGAGAATCACTTGAACCTGGGAGACAGAGGTTGCAGTGAGCCAAGACTGAGCCACT |
| | GCACTCCAGCCTGGCTGATAGAGTGAGACTCTGTCTCATAAAAAAGAAAAACAATTA |
| SEQ ID NO: 511 | ATTCACCTGCCTCAGCTTCCCGAGTAACTGGAATTACAGGCATGTGCCAATACGCCCAGCTA |
| | ATTTTGTATTTTTAGTAGAGGGGGGTAATTAGCTTATTTTTTGTATATAATGAAGTGTCTGCA |
| | GACAGCCACACCCTGGGCTGGTATAATGAGGGGTTTTTTGTAGTCATTAGAGACCCAGACTC |
| | CTTATATCTTTGCCTCCACCACGCTAGTATGTGTTCTTGCCCTCATGGTCATATCTCACCTTC |
| | CTTCCTGGCTAGATGAGGAGAAAGACTGGAGAGTGAGAAAGACATTGACC |
| SEQ ID NO: 512 | TCCTTTAATTTATATTAACTTTCCAACTACCTTAATGTTTGCTAGTCAAGTCCATGCACATCT |
| | TACCCTCGAATGCTCTTACATGTGTGTAGACCTAACTGTCCTATTAGATCCCCTGTCTATTAG |
| | ATACCCAGAGGCATGACCCAGCCAGGTCACTCAAGAGTGGCTCAGCAACTCAGCAGACAGG |
| | ATGTGGTCAAGGGGATGGTCAGGTCCCTGGTTCCACCTCTGTGGTCAGCAACAAGGAAGGT |
| | GTCTGATCAGAGAGAAAGAGTCAGAAAAGCAAGAAGCAAATGTATTATAATA |
| SEQ ID NO: 513 | CTATTCAACACAGTATTGGAAGTTCTGGCCAGGGAAATCAGGCAAGAAAAAGAAATGAAGT |
| | GTATTCAAGTAGGAAGAGAGGGAGTCAAATTGTCTCTGTTTGCAGATGACATGATTGTATAT |
| | TTAGAAAACCCCATTGTCTCAGCCCAAAATCTTGTAAGCTGATAAGGAACTTCAGCGAAGT |
| | CTCAGGATACAAAACCAATGTGCAAAAGTCACAAGCAATTCTATACACCAGATTCCTCTATT |
| | TCAAAGTCTTGAAATAAGTGCCCTAGAAGCAGACTGATCAAAGAGACACAGCCC |
| SEQ ID NO: 514 | TGAACCTGGGTTTTGAGCCCTCTCTTGTAAAATGGGCACAGTAATATTACCTACCTCAGGGA |
| | GTTGTGAGGATTAAACATGAAGTGCTAAGCATAGTGCCTGGTACAAAGACAGTACTCAATA |
| | AGTGCTACCTAAAACTAGTATTCATAGCAATACTGTTAGGATAAAGAATTATCATATATGAG |
| | ATAGTTCCAAATTTTTGTTTTTTTAAAAAAAAAAGAGTTTTATAAGTTCAAGATAATATTTTC |
| | TTACTTCAAAGAAACAATCTCACAACGAGGGAATGGTAAGAATCAGGAGAGA |
| SEQ ID NO: 515 | AGCGAAACCTCATATCTACTAAAAATTAGCAGGGCGTGGTGGCATGTGCCTGAAATCCCAG |
| | CTACTCAGGAGGCAGAGGCAGGAGAATCGCTGGAACCTGAGAGGCAGAGGTTGCAGTGAG |
| | CTGAGATCACACCAATGCACTATAGCCTGGGCGACCGGGTGAGACTATCTCCAATAAATAA |
| | ATGAATTAATTAATTAAAATAATTTTAAATAAAGAAGACAGCATTTTTAACCTGAATTGAGG |
| | AAATTAAACAGATATCAGGAAGAAATGTAAGACGAAACAGAAAATGCCTGCAGTTT |
| SEQ ID NO: 516 | AAAGGAGGCCATTTAAAAATATGAGTGGGAAAAAGGAACAAGATATATACATAAATCATT |
| | CTGGTACAGAATAAAAAATAGTGATGGAAGAGAAGTAACTCATCAGTTTTGGAATCATTAG |
| | TGAGCGGATCATCAGTGTTCTTGGTAGAGAATATTTTCAGACTAAGAGTATCAGGGAAGAC |
| | TGTAAGAAGGAGATGGATCTAGAGGAAAATATAAGATTTGAATTTGTAGATACTTGTGGTA |
| | GGTAGTGGTTTGATGAGGTCAAGATATTCCAGCAATGACTATGTAAGCAAAGGTAAG |
| SEQ ID NO: 517 | GTGCAGTGACATAATCTCAGCTCACAGGAACCTCCGCCTCCCAGGTTCAAATGATTCTCCTG |
| | CCTCAGCCTCCCAAGTAGCTGGAATTACGGGCGTCTGACACCACACTCAGGTAATTTTTGTA |
| | TGTTTAGTAGAGACGGGGTTTTGCCACATTGGCTAGGCTGGCCTTGAACTCCTGGCCTCAAG |
| | TGATCTGCCCGCCTGAACCTCCCAAAGTGCTGAGATTACAGGTGTGAGCCACCGTGCCCGG |
| | CCAGAAACAGTTGTGTTTTTTTTTTTTTTTTTTTTCTTTTTTAAAGAAGAACA |
| SEQ ID NO: 518 | TTCATAGAACATTTTCCAAAATGGATTATGTTTTAAGTCACAAGAAACACTCAATAAGTTTA |
| | AAAGATATTTTAAAAGATTATAGAAGCTGACAACAATGAAAGAAAATATGAGTGAATAGA |
| | ATTATCCTAAATACCTCCTGAGTCAAAGAGGAAATCAAAACTAGAACCACCAGCTATTTGG |
| | AAAATACTGAGCAGAAGAACATTGTCCATGTAAATTAGAATGTGCAGCCAACGCTGCATTC |
| | AAAAGCAAATTAAGAACATTAAAGGTTTTTATTATTAAAAGACATTAAAATGAACT |
| SEQ ID NO: 519 | AATCCCAGCACTTTGGGAGGCCGAGGCAGGCGGATCACCTGAGGTCAGGAGTTGGAGACCA |
| | GCCTGGCCAACATGGTGAAACCCTGTCTTTACTAAAAATACAAAAATTAGCTGGGTGTGGT |
| | GGCACTAGCCTGTAGTCCCAGCTACTCAGGAGGCCAGGAGGCTGAGGCAGGAGATTTGCTT |
| | GAACCCTGGAGGAGGAGGCTACAGTGAGCCGAGATCACACCACTGCACTCCAGCCTGGGCG |
| | ACAGAGACAGACTCCATCTCAAAAAAAAAAAAAAGAAAGAAAGAAACAAACGGTGT |
| SEQ ID NO: 520 | CCTGAGTTTCCTCATCTTGTTGCCAGAGGCCAGGAGGCCCTGCGGGATTGGAGTAGAGGAG |
| | GCTACTAATTTGGTGACTGGCATAGGAGGATGGATGGGAGGCAGCACAGGAAATTAGACA |
| | GCCCTGTACATTGCTGCTGCGATAGACCATCAGCCATGTAACCCAAGGCCAATCCAGTGGG |
| | ACTGGGCCCCGGATGGCACCAATAAAAAAATCCTAAACGCACATGACTGTTTGCTGACAAT |
| | TACAATTTCATGAGGGTAAAATCCATGTGTGGTTACCACTATAAAAACTCACACTCT |
| SEQ ID NO: 521 | CAGAACTTAAAGTTAAAAAAAAAATCCTAGTGCATGTGTATATTTAAAAGCCAGTTTGATTT |
| | TTGTTTTCTGTGTTTTATGGACAAAAAGACTAACACAATCCCATTTTACACGTGAAACCTCTT |
| | AAGCTTACAATGGGAAATAATTTGCCCAAGGTTACCTAACTAGTTAGTTGTGGAACAGGCTT |
| | AGAATCATACTTCAACTTTGTGTCCTTAACCACACCATTCTATTCAGGCACTGTGATAGAAT |
| | TCTCTCTCAGTACCCACGACTGAAGAGTAAGGTAGCAAAAGTGTAGATTGG |
| SEQ ID NO: 522 | TTTATTTATGTAGTCATTCTTTTAATGTGTAATCTTGCTCTAGTAGTTAAAACTCCAGTTTTCC |
| | CCCTTCTTATCCAAGAGAATTAGAGTATCTTTCAAAAAGTATTTACATAGATGTTTGATATC |
| | ATTCGTGATGTTTTTTATATTAATGAAGCAAATTCTTCAGTTTTGCCTGAAGTTCTGCCTTGT |
| | CTCCTGGGCACAGAATTTATTCTTTTGAGAATTTCCTCAATTAATATGAGAATGGTATCTGA |
| | TGTTTTGTCTCCAAGATAGCCTATACAACCAGGGAATAGGTTAATACTC |
| SEQ ID NO: 523 | AACCATCATGTCCTCAGAGTAATTAGATAACCTTTTGCACCCAAAGTTCCACAAGTGGACAT |
| | ATATGAAAACAGAATTTAGAAATCAAGTAATTAAAATATGGTGAATTATAAATAAAATAGA |
| | ATGTTTCTTCAATTACTAAAAGATTAAATTTATGTAATATAATAGAAAATATAACAAAAAGT |
| | CAAAATACAAAAAATAGGAAAACAAAAAATTTTTAAAGGAGAGATCAAGCCAAGTATTCA |
| | ACAGGTGTCTCATAATGTTTCCAAAAGAGAAGAAATAAAATAAAGAAATTTGAAG |
| SEQ ID NO: 524 | TTCGTGAATGGGGCTCAGGAAGCTATTTTTAACAGCTATCCCATGTAAGTTGAAATACATTT |
| | GTTCTTTGGGCTAATATCCAAATTTTTTAACCTTAATGATAATAATCAATAACTTGTTTCTAG |
| | AATACTTTTTGAAGTCTTTATGCACCACCCCCTAAACATATACATGCTTTAATGATATGAAA |
| | CCATTCTCTCTAAATACCAAATTTTCCAGACTTCACTGATTTTCTTAATCTTTTATTTCCAGTT |
| | AAAGTATACTGCCTGTTAATTGCTGCCTATCCCGCTCATGTATGGTTTG |
| SEQ ID NO: 525 | GTTTTTTAATATATAACAAATATAAATATGTCTGGTGCAGTGGCTCATGCTTGTAATCCTAG |
| | CACTTTGGGAGGCCAAGGCAGAAGAATTGCTTGAACTCATGAGTTCGAGACCAGACTGAGC |
| | AACATGAAAAAAAACCCATTTCTACAAAAAATAGAAAAATTGACTGCGCTTGGTGCAGTGT |
| | GCATGTAGTCCCAGCTACTTGGGAGGCTGAGGTGGGAGGATCACCTGAGCCCAGAGGTTGA |
| | GGCTGCAGTGAGCTGTGATTGTGCCACTGCACTCCCAGTGACAGAGTGAGACTCT |
| SEQ ID NO: 526 | TTTCTGCAAGTCATTTTATCTTTACCTGCTATTTCTCTCCTTTACTGAGGCTTAGCGTTTTGAA |
| | ATAAAACCAGACAGTTTTCTAGGCAAGTTCAATGTCCATCCTTACAACAGACTTCACCTTGA |
| | AGAGGAAAGTACAGACCTGGCTAATATGAGAGGAGGGAAAGGGAAAAATAAATGAAGATA |
| | AAAGTTAAATATTTTAAAATATTTTTGTTCTCATTTTTTTTTCTTTTTGCCTTAAATACTTGGA |
| | AGCCAAGATTCGAATTTCATGCTCAGTACCCACAGAGTAAGAATATTTTC |
| SEQ ID NO: 527 | TGAAGTTCAATAAAATTAGGTATGTCTATAGTAAAATAACTAGGAAGTGAGTTTTGAGTATT |
| | TATTATTGAGCTTTATTTAACAGATTTTAATATAATTGTCAGTTTCTATAATTTTTAATAATG |
| | GCTGTGTTTAACAATCAGCCTGCAAAATTCCTACAATTTTTAAATTGACCCTTGTAAGCTGTT |
| | ACAAGCCAGTTCTATCACACTACTGCCTATACTCTTAATTGTGGAAGTGACTTAGGGACTAG |
| | TCAATATGAGTTGAGGTGGTTCTGCTAGCTAATAATTTAAAAAATAGACA |
| SEQ ID NO: 528 | CTCTTTAATTAGATCACACCAGTTTTTGTTTTTGTTGCAATTGTTTTAGGGGACTTATTCATA |
| | AAATTTTGCCAAATTCATGTCCAGAATCATATTTCCTAGGTGTTTCTCAAGTATTTTCATAGT |
| | TTTAGGGCTTACATTAAAATCTTTAGTCTATATTCAGTTAACTTTTCTATATGGAAAAATTTA |
| | GAGGTCCAGTTTTATTTTTCTGCATATGACTAGCTGATTATCTCAGCACCATTTTTTGGATCT |
| | GTGTATGAATCTACAATTATCTTAACTCTTGATTAAAAAAGAAAACTC |
| SEQ ID NO: 529 | GTGAAACATGTAGTGTGTGGTTGCACGGCAGCCCGAGATGTCAGGTGAGAGAAAATTTAAT |
| | TATAACAGATGGCATTAACTATATAAAATTCAGGGGCTCTAGTTTATGTATGAGTAAGCATG |
| | GAAGAATTGAGTGTTTGCCTGTATTGCCCCAGAACATGACTAGACTATGTGAGATTATTGTA |
| | ATTGGTTTCTAATCTAGGGCCAGGTTAGCAGTTTAGAGGGTTATAGGTACATGACAGCTTCT |
| | CAGTAGTTAACTAATAATACTCCCTCTGAATGCTTTTCAAAATGGATGTCCCT |
| SEQ ID NO: 530 | ATTTGACTAAATTATTTTAATTTTACAATTTAATCCAGAACTAATAATTATTACAATTTAATC |
| | CAGAAAATAGATTATTTTAGCACTTGACTCATAATTACATGAAATTAAAAGAATGTATGTGT |
| | ATATATACATATATTAATGTGTAAACATATATATACACATATACATATAAACATACATATTT |
| | TTATATGCCATTTTATTGCATTTTGCAGATATTGTATTTTTTGCAAGTTGGAGGTTTATGGCA |
| | ACCCTGCATTGAACAGTTCTGCCAGTGCCGTATTGCCAAAAATATGCACT |
| SEQ ID NO: 531 | TGGTATTTAGTGGGATGAGAGGTGCAGTAGAAATTATGGACTGGCTTTTCAGCTCCTGTTCC |
| | AACCCATTTATGGAATCACTTTCTGAACTACAGCTAGAATGCCTGTTTGAACTGCAGAGGCA |
| | GAGAAGAAAGAGAAGAAAGAAAGGACAAGAGAGATGAAAGGGAAGGAAGGAAGGGAGG |
| | GAGGGAGGGAAGGAAAGAAAGATGGATGAAAATTACACTTCCCAGCCTCTTGTAGCTAAG |
| | GTCTGACTGCCCCCCATCCCCTGCTTTGTGAGTCTTAGGTGAAGAAAATGAACAGCAT |
| SEQ ID NO: 532 | ACTTAACATCATTCACTTTTTCAAAAGAATTTTAGTGTAAAATACAACATTGTATTTTCAGTA |
| | ATTTGGGGGAAATTATGATTTTTTGACAGTTCAATTGAATTGTAGTCAGTAGTATTACTGTA |
| | GATGAATCGTCTAAAGATTGGTAAGTAATTTTAATAGCAATGGAAAACGATGTTATGTAAA |
| | TACAATGCTCAATAGGTAACAACTATTATTCAATAACAGATGAAGACAGAAAACTGTTCAC |
| | ATACATTATGATATAAAGAGAAATGGAAAAATCGGTTAGGAACACGAACTTTA |
| SEQ ID NO: 533 | TTCAGATTCAAGTCTCTAGATTCTTGAGGTTGGGAAATGTTTCCGGAGCAGCCATGGTTTTG |
| | CATTCATTGGCCATTCAGGTTTTCTGCTTTGCCGTTTCCCCCCCATTCAGGTTTTCTGCTTTGC |
| | CATTTCCCCGGGGGTTTCCTTACCCTCTTGGAAGAACAACCATGCATTAAGTCCATGTTGAC |
| | TGTATTTTACCTGCTGTTTCTATGTGTTTTGCAATAAGGGCTTTTCAAAATACCCATAACTTG |
| | TGGCTAAAAATGAGGTTTGTTCCAATGTGCCTTAGAAAGATCCAGTTTC |
| SEQ ID NO: 534 | ATCTAACAATGGAGCCACAAAATACATGGAGGAAAAACTGAAAGAATTAGAGGGAGAAAT |
| | AGACAACTCAACAAGAATAATTTGAGACTTTGGTTCCAACTTTCAATAATGGATACAGCAA |
| | CTATGCAGAAGATCAGTAAGGAAATGTAAGACCTGGATAACACTATAAACCAACTGGACCT |
| | CACAGATATCTATACACACTTTAATGAACAATAATAGAATATACATTCTCTTCAAGTGCATA |
| | TGAAACATTTTCAAAAAGAGAATATACGTTAGACCATTTTTAAAAACAGCTTCAAT |
| SEQ ID NO: 535 | CAACACCTCAGATCCAAGTTATTTCCTATCCAAGAACACAGAGAGAACCAAAGGGAATCCT |
| | GTGACTGTCTCTCTGAATTTAGTTCACGTGGGGGCTGTGGGGCCAAAACATTGCTTCCTCTT |
| | AAAAAGTCTGACATAGAAACCATTTCTAGCTTCTTGATAGCCCAAGGCTTTCACAAGTGTCC |
| | CTTCTTTGTCACATATCACCAAAGCATGTCCTTCAGGTTTACTGTAAAAATATGAATGTCCA |
| | CTTTCAAATACAGGTAAGAACTCTACATGCGACTTGGAGTGAAATAATTGCTT |
| SEQ ID NO: 536 | TCTGAGGCATACATAGCCTCTGCTTGTCAAAGGATGCCTCAAGACAATAGGATCTAGAAGT |
| | GAACACAGACTCACAAACAAGAAAATGATGCAGACTAACAGGACCACCACTCCTTTATTAA |
| | CTTCAACACTAGCCACTGCAAATAATGGGCTTAAAACCAGCAGAAGACTGATCGTGAAAGG |
| | AAAATAAGCCTTAAAGGACACTTTCAAGTAAACATCAGCTACGTATATTGAAGTGGCTAAT |
| | TGCTCTACCATGTTTCAGCCTATTCAAAGGAAAAGCTATAATAGTTTCATTTTTTT |
| SEQ ID NO: 537 | CTCTACTAAAAATACAAAAAATTGGCTGGGCATGGTGGCACATGCCTGTAATCCCAGCTAC |
| | TCAGGAGGCTGAGGCAGGAGAATTGCTTGAACTTGGAAAGTGGAGGTTGCAGTGAGCTGAT |
| | GTCGCGCCGCTACATTCCAGCCTAGGCAACAAGAGCAAAACTCCATCTCAAAAATAAATAA |
| | ATAAAAAAAAAATAAAACCACTTAAACACATATTATATAAAAAGCATCTGATAATAAACAA |
| | CGGCATCTAGGATTTACACTAAATTAGTAAAAATAATTATTCTCAATTGATGAGAT |
| SEQ ID NO: 538 | GTGAAGATTGTGCCACTGCACTCAAGCCTGGGCAACAGAGCAAGACTCTGTCTCAAAAAAC |
| | AAAAATGGTAAAAATCTTTAGTTTTTAAGAGGTAAGGATGAAGAGAAGTGGGTTAAAGGAT |
| | ACAAACATACAATTAGAAGGAATACATTCAATGTTTGATAGCAGAGTAGGGTGACTATAGT |
| | AAACAAAATGTATTGTACTCAGGAGATGAACACACTAAATACCCTGACTTGATGACTTCTC |
| | ATATATACTTAACAAAATGTTACATGTACCTCATACATTTATACAAATAGAAAAAA |
| SEQ ID NO: 539 | TGACAGTGCTCAGGGTCTAAGACAGGCACACAAGAAACATCACTACTACTGCATGGCCTTC |
| | TTTAGCAGCATGTGGAAATGTTGCAAACCATGGGAAGGCTTGGATTAACCCCATAAAGGGA |
| | CAAAGCAAGGAAAGAACTGGCTGGGATTCCCAACAATCTTGGAGTTCAAAGGACCCATGGT |
| | AGGGCTGTTCTGGAGTGTTGGATGGGATGATAATTATTAATAGTTCCATCTGGTTCCTATAA |
| | AAACAATCACTTCAAAAACATTGATTATTAAAAGCCTCATAGAAAATTTATTTTT |
| SEQ ID NO: 540 | AATTTTTCTGCATTATCCAGTGAGACCAATATAACAAAAGAATTTTACATGTTGAAGAGGAA |
| | GGCAGAAGACCCAGAGTCAGAGTGATGCAATGTGAGAAAGACTTAACCAGTCTTTTGCTGG |
| | CTTTGAAAATGGAGGAACAGACCATGGGCCAAGGACTGCAGGCTGCCATTAGAAGCTGAG |
| | GGGGGAAAAAAAAAAGAGTGGATTCTCCACAAAGGACCAGAGCCCTACTAACTCTTTGATT |
| | TTAGTCCAGTGAAACCCATTTTAGACTTCTGCGTATAAGAATTATAAGATAATACA |
| SEQ ID NO: 541 | TGCCTCAGCCTCCCAAGTAGCTGGGACTACAGGCCCCTGCCACTACACCTGGCTAATGTTTG |
| | TATTTTTAATAGAGATGGGGTTTCACCTTGTTGGTCAGGCTGGTTTTGAACTCCTGACCTCTG |
| | CTGATCTACCCGTCTCGGCCTCCCAAAGTGCTGGGATTACAGGCGTACAGGCGTGAGCCAT |
| | GGTGCCTGGCCTATGGTATTTTTTAATTATGTAAAATAAAGTAAAACTTATGTAAAAATTAC |
| | TTACAATTTTTCTTTAAAAATTTAAAGTAGTAGATTACAAAAAGACATGAAG |
| SEQ ID NO: 542 | GGGCTGGGTGTGGGGTCTCATGCCTGTAATCCCAGCACTTTGGGAGGCTGAGGCGGGTGGA |
| | TCACAAGGTCAGGAGTTCGAGACCAGCCTGATCAACATGGCGAAACCCCATTTCTACTAAA |
| | AATACAAAAATTAGCTGGGCGTGGTGGCACGCACCTGTAATCCCAGCTACTCAGGAGGCTG |
| | AGTCAGGAGAATCGCTTGAATCTGGGAGGCAGAGGTTGCAGTGAGCTGAGAACACGCCATT |
| | GCACTCCAGCCTGGGTGACAGAGCGAGACTCTGTCTCAAGAAAAAAAATGGAGTGG |
| SEQ ID NO: 543 | CCCAGAAAGTTCATTGAGAAAATCACTGGTTTAGACAGCTAAGACTAATCCACTAGTGAAA |
| | TTTATCATCTTCCTTCAGCATCTTTCAACAACATCCAAAAGTCTATATTTTAAAAGATAACCA |
| | CTGCCTTAAATAAATATAATTGTGTTTGTATTGGTTACATTATCTGGTGGCACTGTTTTCAGT |
| | GTAACAAATCATCAAAATGCATCCTCCACATATTTCCAGGGTCCAATTAAGTTGAAAGATTA |
| | TCCAGTAGCTCTTGTTTTCCTCACTACTTAATAGTTATCAAGAATTCTCCT |
| SEQ ID NO: 544 | CAGTTAGTAGGAAAGTATTTTCTGAAAGATAAAGATTTATGTGAATTTATATTAAGACTGGA |
| | CAGTTATCTTCAACCTAAGAATCAGTCTCTAGAATCCAGGATGTTACCTGCTACTCTTACTTT |
| | TTCACACATCCAGTACAACGAACGAACTTAGCAGTTACAAAATATTTTTACTATGCTTCTTC |
| | AAGAAGCAGAACAGACCAAAAGCAAGGTAGAAAAGAGAGTGTACTTTACTTAATAATGGT |
| | CTTGTAAAAGCCAAAAGTATATACTGAGTTGGTGAGTTCAAACTTGGAGGCTG |
| SEQ ID NO: 545 | CAGGACCCCTCTGGAATGACAGTCTTCTGACCTAATATTGCACAAGGATAAGACAGAGAAT |
| | TTCTTTATGTCCAGCTCCTAGCCAGAACAGTAAAGAAAGGTTACAGTAATTATTCTAGTTTT |
| | TATGGCTGGCTTTGGAAAAGAGAAGTTCTAGTTTCTATGACCCACCTTGGGGAAGAGGAAT |
| | TCTGGTTTCTATGACTTGCTTCAAGACAGAATGAAGGGTAAGAGACAGCAAGGCAGAAAAT |
| | CTAAGAGACCTTGGTTCTGAGATTGCTTCTGAGGCCTTCTGACATCCTTTAATTT |
| SEQ ID NO: 546 | TTCTTGGTCCTGGGTATGAGCCCCATATCCTAGGTCACCAACCAGATTGAGAGCCCAGTCAA |
| | AGTTCTTTCTCATGGTGTGTCTAGGACGTCAGAAAACCTAGAGATGTGGCCGGACTCTGAGC |
| | CCCCTGAAAAGGGTTCCCAGACCTTTTGTGAGAGGGAGGCTTTGTCTGCCCAGAGCCCACCT |
| | AGTCTTGGAGTCTTGGAAGGCTGCCCGGCACCTCAGGTAGGCCTGTTTGCACCTCAGCCTCA |
| | CCCTGGTCTGGGTATCCACTCCATACAGACATGTTTTTAAAAATTGAGGAAT |
| SEQ ID NO: 547 | CTCATGATCCACCCGCCTCGGCCTCCCAAAATGCTGGGATTACAGGCATGAGCCACCGTGCC |
| | CGGCCCCCAAAATACATGTTTTTTAAAAAACGATAGTGAGTGTAAAATGGAATCATAAAAA |
| | TACTCCATTAATTCAGAACAGAAGGAAAAAGAGGGAAAAAAGGAACAAAGACTAAATAGA |
| | ATACATATAAAACAAATAGCAAGATGGCAGATTTAAATCTAACCAATGTCAATAATTACAT |
| | TAAATTTCCCATTAAAATGCAGAAAGTATCAGATTGATTTTTGAAAAGCAAGACCA |
| SEQ ID NO: 548 | TTTTGGTGACTCTAAGACATTTTACTTACTACTGTCACTTGCAGGTCAGAGCTTGCCAGCTCC |
| | CAAGAGCTTCTCTAGTGCCAATTAGCTTTCTTTCAAAACAATATATAACGTTTCTCTTTCTAG |
| | TAAAATCTCCAACTTTCTCTGTTCTTCAGACATACAGAGGACCAACCCAGTCTGTGCATATG |
| | TCTCGAATTGCAATTTTGTGATTCCCAAATAAAATGTTTAGAGATTCATCTCTATATTTTATT |
| | TTGACTTTGACAGTACTTAGGCCAAAATTAGGAGTTAAATATAACAGAA |
| SEQ ID NO: 549 | GGGAGCTAGAGTAATATTTGTAGGTTTTACGGCTGGCTTTGGGGAAAAAGGATTCTGGTTTT |
| | TATGACCTGCCTTGGGGAAGAGAGATTCTAGTTTCTGTGGCTAACCTTGAGGGAGAATGAT |
| | AGATCAGAGACGGAAGGGCAGGAGGTCAGGGAAAAGCTTCTGCTTCTGAGGCTGCTGCTGA |
| | GGCCTTCATTTTGGGTTATAGTTTCTGAGCCCCAACAATATATCAAAACATCACACTCTGTC |
| | AAAAATGTATACAATTATGATTTGTCAATTAAGAATAATATTAATAATAAAAAA |
| SEQ ID NO: 550 | CTCTTACGTAGTGCTGCTGGGAGTGTATAATTTTACAAATACCTGGAAATTGGCAATTTCCT |
| | ACAAAGTTTAACGTACGTTTGCCATATGACCCAGCAATTTCACTCCTTGGAATCTACCTAAG |
| | AGACATAAAAACATATGTCCTCACAAAGATATGTGTTTGAGTGTTCAGAACAGCGTTAGCC |
| | ATAGTAGGCCCATACTGAAAACAATCCAAATATCCTTCAACTAGTAAACAGATAAACAAAA |
| | TGGTACTACATCCATGCAAGGCAATATCATTCAACAATAAAAGGGGACAAAAAT |
| SEQ ID NO: 551 | GGCACGGTGGTGCACGACTATAGTCCCAGCTACTTGGGAGGCTGAGGCAGGAGAATTGCTG |
| | GAACCCAGGAGGCGGAGGTTGCAGTGAGCCAAGAGTACGCCACTGCACTCCACCCTGGGTG |
| | ACAGAGCGAAACTCTGTCTCAAAAAAAAAAAGAGGAGGCTAGTGTTGGGAGCAGCTGGTA |
| | ATGGCCAGTGGTCTGCCAGCTGAGAAAGAATGTCGCAGCATTTGGGTTCACACTTTGCCCCT |
| | CACATGTCGCCTTGCTGTGGTTGAAGCAGGTGGGCTGAGTTTTAAAAGGCCATCCT |
| SEQ ID NO: 552 | AATAAATAAATAAAATGTTGTTAAGGGGTGATCAAAAGAGAGTAACATGCATACATTTATA |
| | ATTTTATGACAAATCTGCAAAGTGTGTGTAAGAAAATTAAGGCAATATATTAACATACCAC |
| | ATTGTCCTTGTGATGAGGCTCTGCTTTCTTGGTTCTTTCTACTTTCATACTTTTTCACAAGAA |
| | GCATGTATTATGCTTATAGTTGGAAAAATAATAAACTGAATGTTAAAATGAAAATAAAAGT |
| | AAATAAATTCAGAACTTTTAAAATGTAGTATCATAAACTAAGAGGTGTATAAAG |
| SEQ ID NO: 553 | TGGATGGATCTTGCTGCACGTTTTAGAATGAAGTCTGTGAGTTGCCATGAAATGGGCTTCCA |
| | TGATTATGATATAGTGGCTCATCGGTTTGTTTGGATTCACTTAAGCTGTGGTGACAGACATG |
| | CACTGAGCCAGTGAATGGTGGAAAATATCCTCTAGGGCTCCTCATTAGTCACACTAGAGAC |
| | TCAAACATCATTTAAAACATATAAGCTTCATATGACTTGATCATCCAGTATGTGAATTTTCTT |
| | TCCAAATCAATCAATCGTTCTCTCCCACATGCCTCCATCAAAACAAGTTATC |
| SEQ ID NO: 554 | ATCAATATTTTGAAGAATTAGTTCAGGAAAGTCATTAAATGAACAAAGAACAATAATAATA |
| | GCAGCAATAACACAACTCTGAAGACAAGGGATAAAATTGATTTCCATGGTTGTATTTCCAT |
| | GCTGTATTATTTAATATGTGTGACTGTTAAAAAATGAGGTACAAAAAGAAACAGGAAAGTA |
| | TGAACCACACACAGAAGAAAAGCCAAGCAAAACAGTCAATAGAAACTGCAACGATGAAGC |
| | CCAGATGTTGAACTTACTAGACAAAAGTTTTTAAATTAGCTATTAAAATATATTTTT |
| SEQ ID NO: 555 | TCTACAGAGGGCAGTCTCAGGTTTGCCATGTCAACTCTTCTCTCTCTCTCTGTGTGTGTATGT |
| | ATGTATCTCCGTGTGTGTGTGTTTGTGTGTGAGAGAGAGAGTATGATGGTTGCATACACATT |
| | AGAAAATTAGGAGAGATGCCTGCTCTTTCCATTATTACTGAATATTGTTCTGAAAGTATTAA |
| | ACAATATAATTAAATAAGAAGGAAGAAATCAGACAGAATAAATATTGTAAAGTAGGTAAT |
| | GTTACTTAAGAATAATACAATTTTATATGTGAAAATTATAGAAAATCAGTTCT |
| SEQ ID NO: 556 | ATGCATTACAGAAAGTATCTTTAAGATTCTATTGCAAAAATAAGAAGACTATATTTCTAACA |
| | TTAACGGTAACCATTTCCATTTGGAGATATAAATGTGTGATTTTAAATTTCTACAGTAAGCA |
| | TGAACTACATTTAATAAGGAAAAGAGCTGAAGCTATTTCGAAATAATTTAATGGATCAGTT |
| | CACATGGCCAAATAGATAAGAAATACTCTATTTAAACTCAAAATTTAGTAAGATATTCTCTA |
| | GAGGTCATAGTGCCAAAATTGTATGTGTGTATGTTTATACCCTTACCTTCGTC |
| SEQ ID NO: 557 | CGGTCAATCAGGTCCACCAGGCTCTCATCCTATACACAGCTCCTGGCATCCAGACTAGATCC |
| | TTTAGAATGCAGGATTCTCTGTTTAAAAAAAATAGTATTTTTATTATTTAACTGATTTTCAAC |
| | CAGCAGTCATGATTTATAGAAACAGATTATTTTAGTTTCAATTTTATAAATGACAGAGATGT |
| | TTTAACCATGAATGGGTACAAAATAATAGCGTGAATAAGACCTAGTATTTATAGGACAACA |
| | GGGTGACTACATTCAATACTAACTTAATTGTACATTTTAAAATAACTAAAAG |
| SEQ ID NO: 558 | GCACATTTAAGACAATCTCCCAGAAAGTAAAATTTAAGGATGGAGAATTTGAAAAGAAGGA |
| | AAAGAGAAAGAAGATCTGTCCAGGAGGCCCAATATTTCAAATAGGAATTTCAGACAGAGA |
| | AAACAGAGTAAATGAAGAGAGGAAATTAACAAAAAAATAATTTATGCAAATACATCAGAA |
| | CTGAAAAACGTGAGTCTGCAGGTTGAAGTAGCTCACTGAGTGCTCAGCACATGTGTGAAAT |
| | GAGTCCTACATCAAGAAATGTAATAATAAAAAGAGATGATCATTAACACCTTTTTAAA |
| SEQ ID NO: 559 | ATCCATCCCTCTCTCTCTATATTAATCCATCCATCAATCCATTTCATTATCTATGTATGTATGT |
| | ATGTATATCTATCTATCTATCTATCATCTATCCATCCAGCCACCCTCTGTCTGTCTGTCTGTCT |
| | GTCTATTGGTCTATCATCTATCATCTATCTATCTTTATGTATCTTACTGAGAACTTGATACTT |
| | AACAATGAAAAGAACAGATGAAGACCTCCTCATGGAACCTTTGTTAAGCGATTAATGGAGG |
| | GTGTGTTCCAGCAAAATACGGGAATAAATCAAGTAAGAGGAAAGCATA |
| SEQ ID NO: 560 | AAGGCAATGGCCAATTTAGATTCTGAGACTCTGGTGAGAGTTAAAGCTTTAAGTTCTGTGTT |
| | AACAAATCTCAGTATTTTACGCTTACCCTCTAGAGTTCTTTGAGTTAGCCTCAGTTGCAGCC |
| | ATTTTCTGATAATTTGACCTGAAGGTTAAATTCTTGTAGGGAAGGGTTTCTAAATTAGTTAA |
| | CTGTGTTTAGGATTAATAAATATTCCTGAAAATCTGAAATGTATAACTCTAAGTTTTTCAAA |
| | GCAGAAAGCAATCTTACATTTCTCTTTATTTGCTTTTAAAATAAAATACGCA |
| SEQ ID NO: 561 | AGCTATCATGAGCATCACTGTCCATTGTCAATTTCTTATGATCATGACACTGCTTACTATGGT |
| | TGTGATAAGGTAGCATGGTAAACTTAAAATACACATGCACACCGTCATACACACATTTAGC |
| | TAGACCTTAATAATAGTCACATTTAAAATAATCTGTTGCTACTGAGTAATATCTTCTTCCAA |
| | AGTGTCATAACAGTTACATAAAGCATTCCCAGAGTCAGAACCTTCAGAAAATAAAGCCAAT |
| | TATTTTCTCGATGCAAATTCTCCATGTAAATGTCTTTCCTAAAGATGTGGTCT |
| SEQ ID NO: 562 | ACCCTCAAGTTAACATGATGGAGGCTGGAGATAAGTAGAAGTAGTAAAGAAAGATATTCCA |
| | TTTTGCTGACTTAAGCAAGTCAGATACTAAATAGAAAAAAACATAGGGTAAGGAGCAGGTA |
| | ATTTAGTGGGGATTGGGAAGAGAACTTAATAAATGCAGGTTTGGATAAGTTGAAGTTGAGA |
| | TTCCTTGTGAGCCAGCAAACTGGATGTATAAATCATTCTCTTAGGTTTATATGACTGGAGAT |
| | CACAAGAAAGATTTGAATTAGAAATTTTTAATTGAGAGGTTTCACAATATAAATA |
| SEQ ID NO: 563 | ACATCAAAACAATACCACTGAAATGGCCTGTCAACATTTCTTTGATCAAGGACTGGCACTAT |
| | GGCCCAGAAACACAACAGCCAAGTGACAAGAGAAGATAGTAATCTTCCAGCAATTATTCAA |
| | CTTTTCAGTTCTACTGTGCTCCTAGGTCATACTAGGGAAGCTGAGTGTTTCTCAAGTAGACA |
| | CATGACTGGAAAATTATCAGTTAACCTTACTGAGGCTTCCTAATGCTGCAGCAATGAGACTT |
| | ACTCCACTGGAGTCACACTAGAAGGTTTTAGTCAAATTTAAAAATGGGGCCAG |
| SEQ ID NO: 564 | CAAAACTACACTCAACCTACACATCCATTGTTAGTTGAACTGCAGTATATTTGTACTGTAGA |
| | ATAATTACATAGATTTATATATATACCAGTCATGTATATGCCAGCATGGAAAGATCTCCAGC |
| | ACATATTGAAAAGTGGAAAAAGTAAATTGTGATATTAGGTATAGAATTATGACATTTGGTA |
| | AAAAAATGTGTTTAAAAACAAAATTATATGTATGTATGTGCATAAATTTACACACACATATA |
| | TAAATATATGCATGTGTATATATCTTACATATTGCATGGGAGGTGATCTGGGG |
| SEQ ID NO: 565 | GCAAAGTCACATCTTGCATGGCAGCAAGCAAGAGAGAGAGAGAGCTTATGCAGGGATACT |
| | CCTCTTTATAAAACCATCAGATCTCATGAGACTTATTCACTATCACGAGAATAGCACAGGAA |
| | AGACCCACCCCCAGGATTCAATTACCTCCCACCGGGTCCCAAAGAAACGCAGCAGGCAGCA |
| | CCCACACTCATGCTGACTCTGATTGTCATCGAATCCTCTTCTCTCCAATTCCTAGTGGAGCAA |
| | AAGAGAAATTTCCAAAAAGCTATAGTGAGGAAAACAGGCATAGAAAAATGGGTA |
| SEQ ID NO: 566 | GTAGTTAAAAATGATCCAGGGCTAGAGATGCCCCAGGGACTTTGCAAGAACAAATACAAAA |
| | CCTATTAGAGGGAATGCGTCCCCAAGGCAAGCCTCAAGTAATTCCCATGAATAAAGGTCTA |
| | ATTAGAAAGGTTTATTTAAAAACAAAAAAAACTCTATAATACACATGAATGACAATGGCAC |
| | ATAAGCAAGAACCAGGAGAAATAACAGAAGAATCAAACCTAAAAGATATTAGATAGTGGA |
| | CTTAACAGGCACAGAACAAAAAAACACATATATAATGTTTTAAGAAGTATAAGATAT |
| SEQ ID NO: 567 | AAAATACACCTGTATAGTGGAGCCATCTGGTGGTCACCATTTGAACCAAACCATCAAATTTA |
| | GTATCAGTAATAGTGGGACAACCTCCAGTTTACAGAGAATACAAGAGAAAGGGTTGTCATG |
| | CAAATCTGATAGTACTAGCAAAGATAATTATTCTAAAAAAAAAGTCGTAAGACTATAAGCA |
| | ATAAATAGTTATTGAGCAGCTGAGTTAGGCATACTATAGTCTCAGTTTCAGTATCTAACCAG |
| | CTTCAAGTCACCATTTCAAATGAAATATCAACGCGTGATGAAAGTTAACTTACT |
| SEQ ID NO: 568 | TGAAATAACATGAAGAATTTTTTTCTGACCACAGTGATATGAATAAAATGGTATAAAAGGA |
| | GAAATTCTGGGAAATTCACAATATGTGAAAATTAAACAACATGGTTCTGAACAACAAATGG |
| | GTCAAAAAAATTAAAAGAGAAATTATAAACATCTTGAGACAAGCAAAAATAGAAAAACAA |
| | CATATGAAAACTTAAGGGATGCAGCAAAAGTGGTTCTAAGAGTGAAGTTAGACCTACATGA |
| | AACATATAAACACCTGCATGAAAACAGAAGAAATATATCAAAGAAATAACCAAACAC |
| SEQ ID NO: 569 | CTGTAGAGTTGGATTTCCTCCTCAAAGGGTAGCTTCCCAGCAAAGCCACATTACTGCTTCCA |
| | AAGCAGGGAGCTCACCAGTGTATACTTCCTGATTTTTCTGTCTACTTTCCCTAACTTGGGAC |
| | ATCTGATCGTTCTGCCAGGCCATGCAGCAATTCCTTTCCTGTCCTCCTGTTCATCTGAAAAAG |
| | GCTTAAGCCAGCTCTGCAGCTGAGCTCTTTGCATTTTCTAAGTCCCCCTTCACATACCTGAG |
| | GTCCCCTTGTCTTCATATGATCCCCGATGTAGTCTGGCAAAATTAATGTGT |
| SEQ ID NO: 570 | CTCTGCTTCTCTCTCTGGGGCTGCTTCTGTCGGGAGTGGGGTGGGGGGTCACTCTGTTCCTTA |
| | GCACTGTGGCAGAGCACATGTCAAGATGAAGCTCTGGTGAAGAATTGATCAAAAATAGTGG |
| | CGGAGTGAGATGGAGATTTAAATCAAAGGGCTGATTTATGAAGGCTTCAAAGATTTTTTTTT |
| | TTTAAAGAAAGAACATAGATTAGTTGTTTCTGAGGGCTGGAGGGGACAGAGATAGAGGCGG |
| | CGACGGAAGGATCCTTCAGGTTTCTTCTTGAGGTGATTAAACGTTCTGAAATC |
| SEQ ID NO: 571 | GAACAGGATCTCTAACATGCTAACAGAGAATTGTCAACCCAGGCTGTTCCAGAATGAAAAA |
| | GAAAGACATTTTCAGAGGGAGGAGAACTAGGAATTTGCCACCAGATGATCTGCTCTAAACT |
| | AAATGGGGAAGGAAGTTCTTCAGACTGAAGGAAAATGATACCAAAGGGAAACCTATAACT |
| | TTAGTAATTTTGTAAAAACGAAGAGCAACAGAAATTATTTTAAATGGATAAAAATAATAGA |
| | CTTTTTTGTTCTTAAGTTTTTCCAAATATGTATGATTATTGAAAACAAGAATTGTTT |
| SEQ ID NO: 572 | TTAAAGCAGAGGTGGTATGCTCCTAGTCATGATGGAATAACAGACTAAATTTATACATAATT |
| | GAAATAATAGGGAAAAAGCACACAAAATATAGGAAAGAACAGTTTTCCAACATTGAAAAA |
| | CAAGTAAATAACGGCAGTGATCCCCAAGAATCAGACATCAAAGTAAAAGAGCCCTAACATT |
| | GCTCTAGCTTACTGCCTGGGGAGAGTATGCAGCATAAAACAGATCAAACCAGTAGGACCTG |
| | GGCGTTTTCCCTGAGTTGAAGAGGCAGAGGTCAGGGTTTAAGAAGGCTAAGAATTC |
| SEQ ID NO: 573 | ACCCTGAAAGTCCTAAGAACTGAACATTTTAACCAGAGTTTTATTTAAAAATGGATATCTGG |
| | CTTCTGCATAAATTGAAAAACACAGGCTGTTTGGAAGCCTCCCTACCCACTCCCTGATTATT |
| | GAAACCATTTGTGTGCAATTTTAGCTTAACTTTTCTTTGAAATCAAATATTGCTTTTGGATTT |
| | TGGTGTTATTTTAAAATTTGAAAATTAGGCTCAAACAATATAAACTAAACTGAACAGAAGG |
| | CATCCTGAAATCCTAAAACTATTTTTTAAATTTTATTTTAAAAAATAAAAGC |
| SEQ ID NO: 574 | GTTTTCACCAGGTTGTCCAAGCTGGTCTTCAATTCCTGGGCTAAAGCGATCCGGCCACCTTG |
| | GCCTCCCAAAATGCTGAGATTATAGGCGTGAGCCACTGTGCCCATGCCCACCCTCATCCCCC |
| | AAGTCACTTTAAAATCTGGACTTCATTTCATAAAGATACAGCCCTAAGAAATATTACACATG |
| | CTCAAATGAAATTATTTAAATCCTGTTCTATAGAAAGTGGAATCCAATAAAAGAAATTGAA |
| | GGTAATAATATTCACTTGAATGTGTATGTGTTTTTCGGAATTTAAAAGAAACA |
| SEQ ID NO: 575 | GCCTGGGTGACAGAGTGAGAATCTGTCCCAAAAAAAGTCCCAAAACCAACAAAACAAAAA |
| | CAAAAACAAAACCACCCATGGTTTACTGTAATGTCCCTGCTCCCTACTCTACTGGCAAAGGC |
| | CTATCTACCTTGTCTCAAGGCAATGGCAGTCATTGTCAACTCGGGGAGGCTTCTGCTTACTT |
| | AGTTTTGGGATCTCATGAACCCAATGGTCTCCCAAGTTGTATAATCACTGCTACTTAATCAA |
| | ATGGACAACATTTCTAGCTGAGCCACCTTGCATCCTATGAAATATTGTTACAAT |
| SEQ ID NO: 576 | AAGTATAGCAATATGGGAAATGCTTCTCTTTAATGAACACGAATATGCACCAGATTAGCATT |
| | ACACTATTTGTAGTTACATACTTGGTCATAAGAAAATGAATAACATTGGGTTAGCTATTTGC |
| | ACGTTAGGCTTTTCATACTTTATTTGCAGAAAGAGAGAATTGGATTAGATCATTTGTTTTCA |
| | ATTGATTTCCTAGAAGTATTTATTATGATGCCTAAAATATGAACTCAATATTTTGAAAAATC |
| | CTTTATTTACTATATATGAAAAGATAATACGTTTATTAGGAGATGATTTATG |
| SEQ ID NO: 577 | TGAATCTCAGGCCAAATCCTGAAAACCACTTACTACCTTGACCAGAAAAAGAAAAGGTGTG |
| | AGTGATCAGTTTGTTAAGTTCTGCAAAATGCATGGAATTTTCATGAATGACTCTCATTTTAC |
| | CCAGGCTGTTCCCATCCTAAAAAGTAATATATGCCTTGCAATCACCCCTTCAAGGCAGGTAA |
| | AACCCTGATGGGTATGCAGAGGCCATTTGGTATCTCTGGGTAATATGAATGCAAAGGACTG |
| | AGAATGAAGCACACTGTTTATTTGGCAGCACTGAAAAAGAAGAGTTAAAAAAAA |
| SEQ ID NO: 578 | TAACACCAGGACCTTAGCCACTCTTCCCAAATCTCCCTTGGTCATTTCTGGGAGTCTCCAGA |
| | GTGATGTCTCAGGCATGTCCTGTAACAAATCCACCCATGTTTGCCTGCTCCTGCTGTCACTG |
| | CTGGCTATCCACTGGGACTTCATACTAGACTGCGGGCCCAGATGGAGAACAGGCACATGCT |
| | ATCTCCTTCCACTGTTCTTGGCCCTTCTATCCTCCTATTTCCTCAACAAAAGAAGCAACAGAA |
| | ACTAACAAAAACTCTCTCCTCCTTAAGTTGAAAAAAAAAGGCTTATTGCTTT |
| SEQ ID NO: 579 | TCTGTGAGAAGGCAAACACCTTACACAGTGTAGGTGAGGGGAGATTCACGCACCTGGAACC |
| | ACCCAGGCTGGCCGAATTAAGCCTAAGTCACCTCTGGTAAAGAGGGAAGGAGGGTCCATCC |
| | CTGGTTCAGTTACTGACACCATAGGTGTAGATCACAGGTGCCTATGTCCTGCTGCTCTCCAC |
| | AGGGACACTTTTCCTTGGAAGAGATTTACCAAATGCTAATTGAGTGTTTGTTAAATGTTTAC |
| | TATGGGCCAAGCCCTGGGTTTTTCTCTATGCATTAAAAGTGTGAGTAAAGGGGA |
| SEQ ID NO: 580 | AATCCCAGCTACTCGGGAGGCTGAGACATGAAAATCACTTGAACCCGGGAGGCAGAGGTTG |
| | CAGTGAGCCGAGATCATGCCACTGGACTCCAGCCTGGATGACAGAGTGAGACTCTGTCTAA |
| | AAAAAGAAAAGGAGAGAGAGAGAAAAAAAGAAAATTACTGAAATAAATAAACAGAGGGG |
| | AAGGAAAGAAGGAACGTTACAAAGTTTGTGGGAGTAAACCAAAAATAAAATTCTAAGCAC |
| | CCCCAACCACTGAATGGATTCCCCCCTTGGCCAAGAGGATCCCAAAGAAAACCCGAGGA |
| SEQ ID NO: 581 | AAAGAGAGTATATGGAAACTCTCTATGCCTTCCATTCAATTTTTCTGTAAACCTAATACTGA |
| | TCTAAAAAATGAAATCTATTATAATTAAAAAAAGTTAGGGGGATAATAAAACAAAAAATCC |
| | AGAGAGGTGGTTACCAATATGAGGGTGACACAAGATGAGAAAAAACACACAGACAGATGC |
| | AGCTACATTAGTGATATTCTAGTTCTAATATTGGGTAGTGGTACATCACAGATGTTCATTTTA |
| | CTATTATGCCTCATAACTTGCATACATAAGATATATGCTTTTCAACTACTGAGT |
| SEQ ID NO: 582 | CTACTATGGAAATAACTGCAAGTAGCTAAAGAATGGTAAAAATCTTTGGAGAACAAATTTT |
| | AGGATCAACCTTTGCAAGAAATTGTACATCCCTGTAGAGTCTCCTAGGAAGAAACTAATCTT |
| | ACTACAGTCATAGCTAGAAGAAAGGACAGACCTCACTTCCTACAGCATGGGGGAAAACTAT |
| | CTAGTATCACAGTGAATTTAAATAGGGCTGGTGTAGAACTGGGAATATGTTTTAACCAATTC |
| | TCCAGGTTCTAATGGGAATGGCTATTGAACCACTCTCTATAAAACACAATGTTA |
| SEQ ID NO: 583 | GGTCATCAGAATTCAGTATTTTAAAAAAAAATCCCCTGCAATTCAGATGCAAAGAATTCAC |
| | GGACTAATATGTGGGAACCACAGGTTTCAAGGAACAAGTGTTACAATTGTCTTTCCCAATTT |
| | AAAAGCAAAAAATGGAGGTTAAGCAACATCGCTTTGGTTGCACAGTTAGCTAATTGCATAG |
| | CTGAGATTCTTTCACAGGCAGCTTGACTCCAGAACCTGTGATTTTTATACCATACACTGCAG |
| | TGGCCATTAAGGCCATCTCTGCTGATGATGTGAACAAGTTGTTTAAATATCTTA |
| SEQ ID NO: 584 | GTTTGAGGATATTGGTAACACAAGTGGTTGCTCACTACAGGTCCTCGCTGGAAACAGCACT |
| | AAAGAACCAGGATGTGGACTGACTCCTCTGTGCAAGTCACCGACAGTGCCCATGTTTATACT |
| | GTAGGCCCACGAACCAAGCAGCCATGGCGGCAGGGACGGAGGTGTGCATGGACTCAATCA |
| | CCATCTTTCCCTCACCAGAGCTGACTCAGCTGCCTTCTCTGCTGAAGCCCTAAGTGGAGACC |
| | AAGGCTGAGCTATGGCATCTTAATTATGAGGCATCCCTTCCAAAAGGGAAGACAG |
| SEQ ID NO: 585 | ACCATGCTGCAGAAATCACAGCAGCAATAAACACGGCAGGCAAATGTGTCAACCCCTGGGG |
| | AACTTTAACTTGTTTTCTGCTGTTGTAGTGAGATCAAGTGTGTGTGATTCAACAAGACGGGA |
| | CATGTGAAACTTCCCTGTACCCCTCAAGCCCAGCACAGAGCCTCATCCAGAGTAGATGCTCA |
| | ATGATGACACCTTATAAAACAACAAATAATAGCCAAGACCTCTCCTAAGTCTGCTGAGGGG |
| | TAGCCACATGGCTTCTTAAAAGTCAGAGGCCATTTGCTATTAAAGAAAGCCATG |
| SEQ ID NO: 586 | CCAGCTACTCGGGAGGCTGAGGCAGGAGAAACACTTGAACCGGGGAGGCAGAGCTTGCAG |
| | TGAGCCGAGATTGTGCCACTGCACTCCAGCCTGGGCAACAAAGCGAGACTCCATCTCAAAA |
| | AAAAAAAAAAAAAAAAAAATTCCTGACAATCTCCCGAGTTGTTCCTGAAGGTTGGTTTTCC |
| | ATGCGCTGGTTGTGTGTGTGACGCTGTACAGGAACATCAGTGAGTCAATGCATAAAAGAAA |
| | CTGCAGTAGACAATATACGATAAAGTACAATGCACAATATACAAAAAATAGAAAAAA |
| SEQ ID NO: 587 | GAGAAACCCTATCCTCAAACACTGAGTCTGGGCCATGTTCTCTAATGTGGGCTAACTGCTTA |
| | GTACCCAGTACCTACAAACAAATACTTACTTCACAAATACATACATAAATACATGAATGGA |
| | TATATTTTGTTAACTCTAAAATGCTGTCAATTGAACAATGTACCATCAGTTCAAAAGAGATT |
| | GCTCAGAAGAGGGAAAAAAACCACCACATTGACTGCAAGACACATCCCCAAATCAGAAAT |
| | GTTAAAATGTGCATGGGGATTGGTGGGGGATGTGCAGCTTAGAATTGACTGAGTA |
| SEQ ID NO: 588 | AGAGACCTGGTGGGCAAAATGGAGCAAGAATTTCTGGCCTTTTGACAAACATCTCCACCTA |
| | CTCCTCTTTCCCCATTTCTGAAAACTCCTTGCAGATAAGAAAGGCTTAACAGCAAAACCCCA |
| | TGGTGGAAAGTTTCTTGCTTTTTATACCACTTAATTTTTATAATAATTTTTCCTTCCTGCTATG |
| | ATGTATTCGGCAAAGCCATAATGAAAAACTAAAAACACAACGTAGTTATTCATGTTAAACA |
| | CTTACAGGTGCACAGAGCAACACTAGTTAATGTCAAACATTTCTGGTAACTA |
| SEQ ID NO: 589 | ACAATCTTGATTAGATATATGTATAGCTATGATTGTTCAACTCTGTGTAGCCAAGCATATTG |
| | TGGTTGTATTGTCTAGGAGTCTTGACTGAATTATCTAAAAAACTTCAAAATTGTAGTGGCTT |
| | AATAAAATATAATTGTATAATTTTCCCATGTAATGGTCCTAATTTACAGTTCAGATTGGATG |
| | ACAATCTATCCCTCTCCTATAAGTACTAACTCAAAGACCTTGGCTATTTACCTCTGAGGGTC |
| | CACCACCTCTTAGAAACTTGTCATCAGAGATTGCATCTAGCAAAAGGAAGAA |
| SEQ ID NO: 590 | ATGTGATTCTCCTGCCTCAACCTCCTGAGTGGCTGGGATTACAGGCTCCTGCCACCATGCCC |
| | ACCTAATTTTTGTATTTTTAGTAGAGACGGGGTTTCACCATGTTGTCCAAGCTAGTCTCGAAT |
| | TCCTGACCTCAAGTGATCCACCAGCCTTGGCCTCCCAAAGTGCTGGGATTAGAGGCATGATG |
| | CACTGCGTCCCGCTCAGTTTTTTAAAGCATACAATTCATTGTTTCTCAGTCTTTTGGCTAAGA |
| | TCAAATGTAGTATCTTTTTTTTTTTTAAGACCAGTGGTTCAGAATAGAAA |
| SEQ ID NO: 591 | TAAGTCTGACTAGGGGCATCTTGGAAGTCTTCCTGGAGGAGGGGCCAGTTGAGGAGGACTT |
| | CAGATGTTAAGATGTTTGAGAGGAGAAAACTGGGGAAGTTATGGGGAAGCAACAGGACAT |
| | TTAAGCAACAAGGACATTTAAGTTGGAGATTAGAGGAAAGGCCGTATGCAGTGTCCTTCCT |
| | CCCTAAGGTCCCAACTTGGCTAGGGTAGCTGGCTGTTCAGTTTTCCCTTGGAAGTGACAGAG |
| | CCTCTCTCTGTTTTGTAAAATGTCACCCAGTTTTTGTTTCCAGTTTGAGGTGCATA |
| SEQ ID NO: 592 | TATAAAACTGTGTCAATGGGCTTATAACACAAAAAGGTGCCATATTCATGATAGAATAATG |
| | GAAGGAAGAAATGAAGCATAGTTTTTGTATGCTATGAAATTAAGTTAATATGAACTAGATT |
| | ATCTTAAGTCAAGTTATTAATTGAGGGCCAGGAGTGGTGGCTCATGCCTGAATCCCAGCACT |
| | TTGGGAGGCCGAGGTGAGAGGATGGCTTGAGCCCAGGAGTTTGAGACTAGCCTGGACAGCA |
| | TGTGAGACCCCACTTCTTTCTAAAAAAAAAAAAAAAAAAAAAATCAATTGTTTTG |
| SEQ ID NO: 593 | TCACCCCTTTGGCTTCTTTATCTCCCTTTGGCTTCACGTTCATTGTCCAACACCTCCCAGACA |
| | AGTTGTAGCTCCTATATAAGCAGGATTGGACTACTGGCCTATATAAGCAGGATTGGACTATT |
| | GGCATCTCACTAGAAATTTTCTCCTGCTAAACCATACACTAACATACACAAAGGAGGCATTT |
| | CTCTTCACAGTCTCAAGGTTTTCCCAGCAGAACATCACTATATAATTATGATTAGTTGCTTA |
| | ATCTCCAAAACTTTCTACATTAGTGAATGCGCACATGAAACTTACTAGGCA |
| SEQ ID NO: 594 | TCATTCTATGGCTTAATTCCAGCTACTCAACTATTCTATCTAAACTACTCTAGTTTTTTAGGT |
| | TTGTAAAAAGATGTAGCGTTGTATGTACTCTTTTTCTTTGTTTCTAGTGTTATTTTACAGATT |
| | ATTTTTTACCTTTTATCTTAAAATAAGTTCAGGCCAGGTGCAGTGGCTCACACCTGTAACCC |
| | CAGCACTTTGGGAGGCCAAGGCTGGCAGATCACTTGAGGTCAGGAGTTCGAGACCAGCCTC |
| | GCCAACATGGTGAAACCCTGTCTCTCCTAAAAGTACAAAAATTAGCCAGGC |
| SEQ ID NO: 595 | GGTATTTGTGAACCGTCCTATAAACTTTATAAACTCTCTGGTATTTGGTAGAGGCTCAATAA |
| | ATGTCATGGTCCCCACCCTTCACCCTCCAGCCTTTCCACCTATATAAATGCAGGATCTCATTT |
| | AATCCTGGTTCTTAAAGTGTCCTGTGAAGCTTAGTGTGCCAGCCGGCTATTGCTCTGGCCTG |
| | ACATTCTGCCCCCAAATTGTGTGTGTGTTTGTGTGTGTGAGTGCACGTGTGTGTGTGTGTTTA |
| | GATCTTTTACAAGGGGGAAAGTGTTCATTATGTCTAATATAAAATACAGA |
| SEQ ID NO: 596 | CCAGGGCCGGGAAGGTGCTTCCACAATGCCATTAGGTAGACAGGCTCCTCCTGTCTTTCTTT |
| | CTCCCATCTTTAGTGTGTAGCTTTTATCTTCATGCTTGCAAGATAGTGAAGTACATTTAGATC |
| | TAATGTCTACATTATAGGCAGGCACAGAGTGAAGCGCAGAAAGCAATTAACAGTTGTATTT |
| | TTCCATCTGATCAGGAAACAGTAACTTTCTTGAAAGTCCCACACAGGAGACTCCTCACATCT |
| | CATTGGCAAGAACTGGCTCTCATAGTTATATTTTTGTATACAAAATTATTTA |
| SEQ ID NO: 597 | AGTGGCTCATCCCTGTAATCCCAGCACTTTGGGAGGTCAAAACAGTGGATCACATGAGGTT |
| | AGGAGTTCGAGACCAACCTGGCCAACATGGTGAAACTCCATCTCTACTAAAATACAAAAAT |
| | TATCTGGGTGTGGGGGTGCATGCCTGTAATCCCAGCTACTCAGGAGGCTGAGGCAGAAGAA |
| | TCTCTTGAACCCAGGAAGTGGAGGTTGCAGTGAGCCAAGACCACACCACTGCACTCCAGCC |
| | TGGGCAACAGAGCGAGACTCTGTCTCAGAAAAAAAAAAAAAAAAAAACCCATGCCT |
| SEQ ID NO: 598 | GGAGGTTCAGCAATTCCTGTGTGGTTCTAGGTGGGTACGTGGATGCCTTCTAAGTGTGGTCT |
| | CAGAAATCCAAGGTCAGAGTAAATCTCAAGTCATTGAAGCTCCAGTTTTTGTTAGAGTGGAT |
| | GTTTGGGCTACATCACTGCCCTGAAATTGCTGTGATTCGACAGGAACTGAGGCAAACCTTCA |
| | GCTATAAGATGAAGACCTAATGCATGGTGACTATAGGGAATAATAATGTATATTTGAATTTG |
| | CTAAGACAGTAGACCTTATGTGTTCTCACCACACACACACAAATGGTAACTA |
| SEQ ID NO: 599 | GTGATGGCGTTACAGTCGACAGTTCCATACCCTTCACTTCTGAGACAGTCCATGTTCCAATA |
| | GTCCTAACTGTGAAAAAGAATTCTTCTTAATATTCTTTCTATATATCCTTGTCAATGCCCTTA |
| | ATCTCATTATCCAAGTTATCATTTTTCTCAATCAGATGAATTAAACAAACACTTGAGTCCCA |
| | ATTATGTATTAGGCTTTACTCTGAACAGTTTCTTTACCATAGCCATTAAAATCATTACTGACT |
| | CCTTAAAAATGACCGCCCACCCACTTTTCTCAGTTTAACAATGGACTTCC |
| SEQ ID NO: 600 | TTATGCATACATACCTGTGATAAAGTTTAGTTTATAAATTAGGCACAGTAAGGGATTAACAA |
| | CAACTAGTAATACAGAACAATTAAAACAATATATAATAAAAATTATGTGAATGTGGTCTTT |
| | CTCAGGATACATTTTCAGACCACATTTGACTGTGGGAATACATTTTCAAACCACATTTGACC |
| | GTGGGTAACTGCAACGGTAGAAAAGGAAGCTATGGTTAAGGAGGGACCACTGTATATTGTT |
| | AGGTTAGTTTTTTGTTTTGCTAGGGAGGTTTTTCTATGTTGAAAATGTATAGAT |
| SEQ ID NO: 601 | TTTGGAAGGCTGAGGCGGGCAGATCACCTGAGATCCGGAGTTCAAGACCAGCCTGGCCAAC |
| | ATGGAGAAACCCCATCTCTACTAAAACTGCAAAATTAGCTGGGTGTGGTGGTGCATGCCTG |
| | TAATCCCAGCTACTCAGGAAGCTAAGGCAGGAGAATCACTTGAACCCGGGAGGTGGAGGTT |
| | GCGGTGAGCCGAGATCGCGCCATTGTATTCCAGCCTGGGCACCAAGAGCGAAACTCCATCT |
| | TAAAAAAAAAATAAAAATAAAAAAATTTTAAAAAGAAAAAAAGAAAAAGAATTAAA |
| SEQ ID NO: 602 | CTGCCTCAGCCTCCCGAGTAGCTGGGTTTACAGGCATGCGCCACCACGCCCAACTAATTTTG |
| | TGTTTTTAGTAGAGACAGGGTTTCTCTGTGTTGGTCAGGCTGGTCTCTAACTCCCGACCTCA |
| | GGTGATCCACCCACCTGGGCCTCCCAAAGTGCTGGGATTACAGGTGTGAGCCACCGCGCCC |
| | GGCCATGAAAATTGTTTTAAGAGCTATATGACTCTGAGAAGCTTATGAAGTACTAATGAAG |
| | ATTTGATGAACTCTCAAACAAATTTTTGTTTGCTAGAAATATAATTCTTGGGGA |
| SEQ ID NO: 603 | AAATGTGCAAAAACTATAAAACTCACTGGTACAGTTGATACTCAAAGGAGAAAGGAATCAA |
| | ACCTTATGGCTACTTAAAACCACCCAAATTGCAAAAATAAGATGGGAAGTAAGAAACAAAA |
| | ATTTATATAAAACAACCAGAAAACAATGAAGTGACAGGAGTAAGTCTTACTTATCAATAGT |
| | AACCTTGAATGTAAATGGATTAAATTTCCCATTTAGAAGATAAAAACTGGGTTAATAGATTA |
| | AAAACAACAACAATGAGGCCTAACTATATGCTGTTTATGAGAAACTCACATACTG |
| SEQ ID NO: 604 | TCTGTATATACATTAAAAAATATGTTTTTTTAATAGAGACGGGGTCTCTATTAAGTGACCTCT |
| | ATTTTTTAAGTGAGACGGGGTCTCACTGTGTTGCCCAGGCTAGTCTCAAACTCCTGGGCTCA |
| | AATTATCCTCCCCACTTGGCCTCCCAAAAGGATGGGATTACAGGCATGAGCCACTGCCCCA |
| | AGCCTAAAATTTTTTTAAGTACCGTTAGAATGTAAGGATTCTTTTTAAAAAATTTGATTGTGC |
| | AGGGTTGGTTATTCAACCAATATGCAATACAATATTCAATACTGTATATTC |
| SEQ ID NO: 605 | GTGAGGCTGGCTGGAGCAGCCACATTTTCCTGGCAGCCCTGTGCTTTCTTGGGCTGGTCCTT |
| | TGAGGGGGCCCAGTCCTCCTGAGGCTGTGGCCTTGACCTGCAGAAGCCATGCTAGAGTCCA |
| | GCTGTCTCTTGGTGCAGGGGTGTAAATGGCACACATTGTCAATGTGGGGTTCTCCTACCCCT |
| | CATCCCCCAGCACCCAGAGGGAGAGGGTGCCGCTGCGGCAAAGAGGCTTGAAGTTGGTTTG |
| | GTTTGGGGGATTTCTGTTGTGAGTTTTTAAAAATTGAGGTTAAATTCTCGTAAC |
| SEQ ID NO: 606 | TGGACCAAGCATCTTGTCTTAGCACCATAGGGTCGGAGAGGAGCCTATGATGTTTGTTATCT |
| | CAGCCTTCAATGGAAGAGACAAAGCTTTGTGTGGTATTTTACATTTAGAAACTATCAGAAAC |
| | ATGTATATTTATATTAGTTTCAGAAATATATACCTGTATCAAGCCTGTTATATCAGCTGCAA |
| | CAGAGATGTAGCAATTTAAAAATTTTAAGAGTTGCCTGGGTATATGAAACAAAAGATATAT |
| | ATAGCTTTAGCTGGGAAAAAATTTCAAAAATCCCATTGAGTTTAAGTTACACC |
| SEQ ID NO: 607 | CCTGGCCAACATGGCGAAATCCCGTCTCCACAAAAAATACAAAAATTAGCTGGGCATGGTG |
| | GCTTGCACCTGTAGTCTCAGCTACTTGGGAAGGCTGAGGCAGGAGAATCACTTGAACCCGG |
| | AGGCAGAGGTTGCAGTGAGCCAAGATCACGCCACCGCACTCCAGCCTGGGCGACAGAGCA |
| | AGATTCCATCTCAAAAAAAAAAAAAAAAATGGTGTGAGCTCTGTAGCTTAGTAGGTTGCTT |
| | CCATGGCAGATGGGAAAATATCGAGTCATCCATTTTGATTTTAAAAAAGCAAGGCAC |
| SEQ ID NO: 608 | AGGCCACGCGGGCATTGAAATCACGACTGGTGTGTGGACCAATCCCAGGATCCTAAAAAAG |
| | CGAAAGGGGCGGGTTTATGCAAAACAAACTGAGCAGGGAGCAGGCGGCGGAAGAAGTAGA |
| | GGACGTTTAAATAGGGCTGTTTCCAAAATATAGCCACTACTATAAAGAAAGAATAATGAAA |
| | ACTGTGTTTCGGAATTATAATGTATTGGGAGATAATTTAACATTTAGTGCCTGGATAGTTAC |
| | CATAACTGGTTGGAAGATGGGAAGGATAAGGCCGCCGAGGCGACCGAAGTAAAGGT |
| SEQ ID NO: 609 | AGAAATAAGTAAAACATAAGCGTGTTAGATGGTGATGAGGACTTGGAGTAATCAAGGGAA |
| | CTAAGCCATCCTCTGAGGGAAGATCATGCCGGCCAGAAAGTACAAGTCAAAGAGTGCTGGG |
| | GCAGAAGCAAATCTGGTTCAAGGAAGAGTCAAGAGCCAGTATGGGCCAGGCCCGGTGGCT |
| | CACGCCTGTAATCCCAGCACTTTGGGAGGCTGAGGCGGGCGGATCACCTGAGGTCAAGAGT |
| | TCGAGACCAGTCTGACCAACATGGTAAAACCCTGTCTCTACTAAAACTACAAAACATT |
| SEQ ID NO: 610 | TAATACTAGAATAGTATATCCAGTGAAAATATCCTTCAAACATAAAGGAGAAACAACGAAT |
| | TCCCCAGACAAACAAAAGCTGAGGGAGTTCATCAACACCAGACCTGTCCTACCAGAAATGC |
| | AAACGAGAGTTCTTCAATCTGAAAGAAAAGAATGCTAATGAGCAATAAGAAGTCATCTGAA |
| | GATATAAAACTCACTGGTAATAGTAAGTACACAGAAAAGCACAGAATATTATAACACTGTA |
| | ATTTTGGTATGTAAACTGCTCGTATCTGCAGTAGAAAGACTAAAAGACGAACCTGT |
| SEQ ID NO: 611 | TGGTTCCTGCCTTAACTGATGACATTCCACCACAAAAGAAGTGAAAATGGCCTGTTCCTGCC |
| | TTAACTGATGACATTCTCTTGTGAAATTCCTTCTCCTGGCTCATCCTGGCTCAAAAGCCCCTA |
| | AAACGGCCCCACCCCTATCTCCTTTCGCTGACTCTCTTTTCGGACTCAGTCCGCCTGCACCCA |
| | GGTGATTAAAAGCTTTATTGCTCACACAAAGCCTGTTTGGTGGTCTCTTCACACGGACATGA |
| | ATGAAACAAAACTCTTGGCATCCAAATTAAGCCATCAAAGCCTCAGCTTC |
| SEQ ID NO: 612 | CTTTATATGGTTACTTTAGGGGCACATATTATGATATATCAAAATCAATGTAAATAAGTCAT |
| | GTTTGAACAGAGCCGTAGTCTGCAAGAATGAAGAAATCACTAATAGAATTTCCTTCCTTGTT |
| | CTAAGAAAACATTTAGACAAGAGGAGTTCCCAACAAATGGACTGTGACATTGGCGCAGAAG |
| | TGGAAAAGTGAACTGCCTAAAAAAGGACCAGCTGAATGAGGAACAGGGAGGAGGCAATAT |
| | AGAGGTAAAAAAAAAAAAACAAAAAAAAAACAAAAAAAAAAAACAGAAACAAAAG |
| SEQ ID NO: 613 | CAGAATGGACTTGGAGAATCTTTTGTATCAGAAAGTAAAGTTCTCAAAGAATGATAGGGAC |
| | ATGTTATAAGGACACAGGAACCAGCATAACGGGGCCCCCACTGGCCAAATGCAGACAATGT |
| | GTACATAGTAAGACAAATGAATGTAGATACCATCTTAGCCAGGTGATGAAACAAACTGGTA |
| | TCGCACTACTCCTGCTATGATGCAGTGAGAACCCTTCTGTGATACTCCCCTCAAAAATGAAT |
| | AAATCAAAAGGATCACAGAAAAACACAAATTGCAAGGCATTCTATAAAGGAACTG |
| SEQ ID NO: 614 | ATCTGCCTCACCCCACGGAGGAAAACTGAGTCCAGGAGACAATCCCCCCCTAAATTTACCA |
| | ACTGTTTCCCCCCTTCCAGAAGTTTCTTGAGAGCAGGAGCCATATGGATTCGTCTCTGCATG |
| | CCCAGCCTGAGACATCAGTGCCTAGGACTCAGGAGGCATACCCAGCACATGCTTGTAGAAG |
| | AAAGAAAGAAACAGGGAAGGTGTTTTTTGCTATTTATCGCATATTTAATTTCATCTTTATTAT |
| | ATTTTTCCTGATTGTAACAGGATGATGTCTTAGGTGTGAAAAATTAGAAGTGG |
| SEQ ID NO: 615 | ATTTTTAAAATGTATGCTTCACACAACACCAATAAACATCTAAGAGATGTGATATAATCCAA |
| | GGCAAAGAAAAGTAAGAATCAGTTAATGTCTATTTTATCTGCACAGCATTACGAACAGAAA |
| | ATTGTAATAAAAAAAGGTTTAACCAAACTGAAAAAAAGAGAGACAAAATCTTGGTTTTAAT |
| | TTGAAAAGATGATTGGGAGTGATCTCAGAAGAGAGTTCAGCAGGAAGCAAGGAAGAGGCT |
| | GTTGACCTTGAGTGCAGCCTCAAGAATGGAACCTTTTTGAAATATTTTAAAGGAAA |
| SEQ ID NO: 616 | TCCTGGAAGTGGGAATTGTACTTTTACTTTTTACTTTGTATAATAAATTATTCTTTTTAAACG |
| | GTGGGAGTATGAATGGTAACTTTTAACATTTTTCTTTTGTGTTTTTTACTGCCAAATATGCTA |
| | AACAATGAATCTAAGTGGTAAAGCTATGTGGTCTTCCAGCTATAAAATGCATATTTACATGA |
| | AAAATCTCAGATAACAGGAGGCAAAGATTCTTGTTCAGATTATTTATTTCATGCAACTGAAA |
| | ATGTTTCTTCTTATAGTCTCTCATAATCCACATTTAAAGTATTACATATA |
| SEQ ID NO: 617 | TCACATTGTATTAGGTATTATAACTAACCTTGAACATATATAGACTTTTTCTCTTATTATTAT |
| | ATTGTATTATTATGATATAATATAACAACAATTTACATAGCATTCACATTGTATTAGGTATTA |
| | TAACTAATCTAGAGATTATTTAAAGTGTACAGGAGGATGTGCACAGGTTATATGCAAATAC |
| | TATGCCATTTTATATCAGGGACTTGAGCATTTATGGATTTTGGTATCTAAGAGAGGTCCTGG |
| | AGCCAAGCCCTCACAGATACTGAGGGACAACTGTATTTAGAATAAATGTTT |
| SEQ ID NO: 618 | TTTTTTTCACCTTAAAAATATTAATGTGGGGCTTACAAACTCATAAATCAGTTACTGGTCAA |
| | ATTTAAAAAAAAAATGACGTGCCTTGGCTAGTGCAAACAGTAAATCAGTTTTTTTACTGTGT |
| | TTGTTTGATTCGCTTGGAATCTAACAAACATTCTGTAAGAAGTTACCACCGAAGACAGGAG |
| | AAACAGATACCTGGCACTTCCTTCCTTACTAATGAAATGTTATGCATTTCAGTACTTTCTACG |
| | TGAGGAAAAGCAATATTGCATAAGAGTAATTAAAGTGTTCACAATTAGGTCC |
| SEQ ID NO: 619 | AGCCTGAGCAATATAGCAAGACCCTCACCTCTTAAAAAAAAAAAAAGTAGATTAAAAAAAT |
| | ACCACAATTGCTCAGGTAGATTGAAAAACAGGCATATAGTACTTATGGTACAGGACCAGCA |
| | TGCATGCATGCATGCATTGATTGATTGATTGATTGATTGATTGAGACAGGGTCTCTCTCTGT |
| | CTCCCAGGCTGGAGTGCCTGGCCTTAAGTGATCTGCCCACCTTTGCTTCCCAAAGTGCTGAG |
| | ATTACAGGTGTGAGCCACCATGTCAGCTGGCGAGGCTTTTTAAAAGATAGTTCC |
| SEQ ID NO: 620 | CATCTTGCTGTGTCCTCACATGTCAGGGAGATAGCTTTCTAGCTCTCTTATTTACTTTTATAA |
| | AAGGAATTCCATCAGGAGGATTCCACCCTCATGACCTCATCTAAACCTAATTACCATCCAAA |
| | GGCCCCATCTCAAAATACCATCACATCATAAGTTAGAGTTTCAACATGTGAATTTTGAGGGG |
| | ACACAAACATTCGGTTGTTGGCAACTGTAAAGTTGGAGCAACCCCCATACAGAATTCTTGGT |
| | CTCTGTGGTAAAAGATATTATAGTACAAAAGCCAAGCCTCTGAACTTAAGT |
| SEQ ID NO: 621 | CTTTTACTGTCTCGTATCTGGAATCACCATTTTGCCAAGAAACGCTGGTTTCTTTCAGTGGGG |
| | AGTAGTATTTGGAAACCAAAGTCTGGGTGTTTAGAATGCTCACAGTCCTAGGATGTCCTTGT |
| | TTCCAGGCCCTTTGAGTAGACAGCATTAAAAAATACATGTATTTCAACAGTTGACTTCATAT |
| | TGATATCTCCAATTCAAATTTAACATTACAAGGTTTTTCTTCAATTTAATTTATATTTATTGC |
| | CTTACATTAACAATTATGGCTGTTAATAAATAACATTTATTCTATATCCC |
| SEQ ID NO: 622 | ATACCCTGCTCATGGGCTGAACCATTCAAAGTAACTTCTCACATTATAACTTTAACGTTTTA |
| | ACGGCTTGACATGACATTAACACTATGTATTTTCTTATATCATTCTTATGAGACAAGAAGAA |
| | TCAGTCAATTAAATATTTAATGAACATCTGTTAAGTTCAAGGTAGTACTCTAATTTGTAGAA |
| | ACATACAATTTACCAAGACTGAATCACAAAGACACAGAAAATCTGAACAGACCAATTATGA |
| | GTAAGGAGATTGATCCAGTAACCAAAAACTTCCCAATAGAGAAAAACCCAGCA |
| SEQ ID NO: 623 | ATCCAGTCACCTCCCTTCAGGCCCCTCCTTCAACACTGAGGATCACAATTTGACATGAGATT |
| | TGGGCAGGAACACAAATCCAAACAGTATCGCCATGAACAAACAAGGCTAACTCTCAATTTT |
| | CTGGAAGAGTTTCTAATCTGTTTAGGGCATGGACTGTTCTTGGCCAGAGGTTAGGCAGTCAT |
| | GATTTACCCCTTCTTCTGGTAAAAGAAGGAAAGCAACAGTACATATATTATGGTACTACCAG |
| | GGAAGGAAACGTAAGTGAGAAAGCAATAAAATTTCATGAATAGTAAGCATATC |
| SEQ ID NO: 624 | AACGTTTGATTGTTCCTTTAAAAATTCCATTAGTCTCACTTTTCAGGAATTTGTCTCCAGCTT |
| | CCTAACATTTCCTAAAATTGTTCCCTGGAAGTAAAAGTACTGCTTCTTTTCGAAATGACCAC |
| | TTATTTGAGTACTTCCTTGCTCCAACAATGTCCATCCGCAGTGTCCTCAGATGTCACCCAAA |
| | CTATTCTGAAGACAAGGTTAATTTTAAGGGGATTTCAGAGTCAATCATATAAACAGTTTAAA |
| | AGTGGGATAGAATGGAGGGAAAGTGCAAGCACTGAGTGTATTAGATACCTA |
| SEQ ID NO: 625 | CAAAACATTTAAAAAAGAGATTCAAATGAGAATAGCAAAAGCTAATAAAGTGTTAAGGTG |
| | ACAAAAAACTACTCATACAGAATAATCTACTACTATGATGGAGGTTTTATAGATGCAAGAT |
| | ACATATTCACACATATATTTAACATTTTTCCCTTTGGTAGGTAGAAGATGAAGTAGAATACC |
| | AACATAAGAGCAATATGACTGGTTTCCAGTCTTTGATCTGCCACTAGTTTGTGACCATAGGA |
| | AAACCATCTTAACTTTTTTTCTGAACCTCTGTTGTTTTCATTTAAAAGAATGGAG |
| SEQ ID NO: 626 | CCAGCCTGTGATAGAGTAATTATGTTGCTAGATCCAAAGTAAACAAAAAGTTTCCCATAAG |
| | GCTTGGTATTGCTTACAAGTTACAGTGAATTCTAGGAAGACTATTATAAATTATAATTTAAT |
| | TTATATATATATTTAGAATTTTCATGTGTTTATACTCTAAGAGCCATCTGAATAGGAATTTGC |
| | TTGGGGCTGTCAAAAAGTGAAGTATTTTCTTGGAACAGTGAAATATCAGAAAAAATTATCTT |
| | CATTTGTCTGGAAAGCAGCATTAAGGGGGCTCTTATATTACATGAAAATATA |
| SEQ ID NO: 627 | CTGGGTTTGAGTATCTGAGGAAAGCTGTTTTAGAGCAGAGCAGAGACTCAAAGCATTGGTT |
| | CTCATTCATTACTAGTCTTCTGATTTTGAATCAGGCACATGGCTTCTCTAAGCCTCAGTTTGC |
| | TAATCTGTAAACAGGAGAGTAATAGTACTTGCCTCTCAGGTTCTTTTTTTTCTTTTGTCAAAA |
| | GCATTTTATAAGCTACAAAGGGGTCTAGAAATGTAGTTTATTACAATCGTTGTTACTTACAG |
| | GTCTGAATTTCCTGCAGGAAAAGGGGATAGAAAAGTATAAGAAATAATGGT |
| SEQ ID NO: 628 | AGGATAATGACCAAGAGACGGTTCTGGTCCTCAAGCTTAGTGATGGGGTTCAGGGAGGCAG |
| | CCAGTTTAGCACAGGAGACAAACATATAAACAGATAATTATAATCAAACGTTATGGGTTTG |
| | GCATTAGAGGTATAGTGGGAAGAGTGATCAACTCCATGTGAGGGAAAAGGCAAAACTGCA |
| | GGGAGTGGGGGGGGGGCGTCTGTTAAGTAGGGTTCTAAAAGCTGAATAGGAGTTTGCCTGG |
| | CAAACATGGAATGGGAGTGGGCAGAGTGATACTCCAGGGAGAGAAAATAGCAGGTGG |
| SEQ ID NO: 629 | TGACTGTTGTTTTTTACATAAGCCAAAGTTTAGAAGGTGGACTCAAGGATGGACCAGATCA |
| | GGATGGGCCAGGTCATGCTGTGATCACAGACAACCCCAAAATCTCCTGACTTATGACACCA |
| | AAGATGGACTTGCTGTTCCCACTTCATGCCCATCACAGGGTATATTCTTTGGCTCCATGCGG |
| | CCTTCATTCTGGGACACAGGCTGCTAGTACAGCTGCTGCCTGGGGCAATTATCAGTCATTGT |
| | GGCAGAGGGAAAAGAGGTCATGGTGAAGCTCACATTGGCTCCTGACGCTGCCAG |
| SEQ ID NO: 630 | AAAATACGTTTTTTTTTTCAGTAGGTGGATCAACCTCAAATTTTAATATAAAGCATTACTTAA |
| | AGGAGAATATGGGGACATTCATGACATTTCTTATATGTACATAAAACTTCATGAAAATAATT |
| | TAATGCTATCCAGCAGTTTATTTTAGAAGTACTGGAGGCTAGGCATGGTGTCTTATGCCTGT |
| | AATCCCAGCACTTTGGGAGGCTGAGGTAGGAGGATCACTTGAGTTCAGGAGCTGGAGACCA |
| | GCTTGGGCAATATAGTGCGACCCCATCTCTACAAAAGAGAAAAGAAGTACTG |
| SEQ ID NO: 631 | GCTTTCCCACTGCTCACTCTCCTCGCTCAAAATTCAAGTCTCAACCGAGTCATCTCTGTGACG |
| | TCACGTTGATTTGCATAAGATTCCCCAGCGTCCCAAGCGAATATTCTTATGGTTTTCAAAAC |
| | CTGAATGTTTGACACGGGATGTTCCAACAACAAGAAACCTCCTATGCAGATGGGCCTTAAA |
| | TACGGCTGGTGGAGTGGGAACACGTCGTATACACGGACACACGGGCAGGCACTCACCCTCA |
| | ATGTAATGGTAGTCATCATCCGTGGGGGAGCGGGGCGCGAACAGAACCTTTCC |
| SEQ ID NO: 632 | GAGCCAAGTGAACAGAACCCTGATTTTGATCCCTTTTCTCTCAAAAGCCCTTCGCAGTCTCT |
| | GAATTAAGTCTATTAGCATGTTCCTCCCATAGTGCTTTGCTTCATATCAACAAAAACCTAGC |
| | TAAGTGAAATCAGCAACGATATGCAGAAACCACCTACGCAGGTCACAAACATCTTTCTATG |
| | ATTGTATAATTTTCAAGCAAGCAATAAGTGAAGATTTTTCCATAGGCCCTAAACTCACCTTT |
| | GCGAAATAGGAAGCTGGTTTATTGGGAGTGATGAGCAGGGGGCGTAACAAATT |
| SEQ ID NO: 633 | TTAAAAACTTTGAGACATGAGGCAGGAAAATATGACTGACAAAAGGAATAGGCAGATAAT |
| | TGATAAAGATGCACAGATGATCCAGATGATGTTAGCAAACAAGTAATATTACAAATAACTA |
| | TCATAATATATAACTATGTAAATATTTAAAATACAGAGAAGTTGTATACAGTGAATAAAAG |
| | GAGAACTGGAATTAAAAAAAAAAAAAAACAAGGAGACATTTAGAAACAAACAACTGATCC |
| | CTGAAATGAGCATCAACGGGACAATGGCCAATACAAGTCTGTTAAGGATGTGGAGCAA |
| SEQ ID NO: 634 | TCACTCTGTGCCTAGGCTGGAGTGCAGTGGGGTGATCTCAGCTCATTACAACCTCCGCCTCG |
| | CGGGTTCAAGCAATGCGCCATGCCTCAGCCTCCCGAGGAGCTGGGATTACAGGCATGCACC |
| | ACCACGCCCGGCTAATTTTTTTGTATTTTTAATAGAGACGGGGTTTCTCCATGATGGTCAGG |
| | CTGGTCTAGAACTCCTGACCTCAGGTGATCCGCCCGCCTCGGCCACCTGGTAAGACTGCTGT |
| | TAACATGTTGGTATAAAACTGCACAACTTTTTTCTAATTACAAAAATAGATAA |
| SEQ ID NO: 635 | ATGTAGAATTGCAGCACAAGATCAATCTGCAGGTAGACATATATTTTTCCATGGTTCTTCGC |
| | AAGGCATGCTGAGCATCTTTGGTGAGTTTGTCAACTTCTGTCAATAATACCACTGAGTCAAA |
| | GAGAAACAAAACAAACTTTAACTTGAGTTCACTCAAACTTTAACTTGTCTCTTTCTTCCTTCT |
| | CATTATCCAGGATGAAATCCTAAATAATAAGCCTGATATACTACTCTTCCCATTGGCAAAGA |
| | TAAAGTTATAAGTATGGTAAAATTACTTTATTATTTTAAAATGCTACAATC |
| SEQ ID NO: 636 | CTGGGGGAGGAGCATTTCAGAAAGAGGAAACATAAAACAGCATTCCTAGGGTTAGAAGGA |
| | GCCTATAGGGTTCAATAACTTGCAGTGTGGTTGGGGCTTATGGATGCTGGATGAAGATAAG |
| | TGAGAGAGGTTTACAGGGACAGCATCATGTCAGGCCTTGAGGGCAAGGTAAGGAGTCTGGA |
| | TTTTATTCCAGATTTGATGGGAAGCCTTTGGGGGATTTCGATTAGGGCAATGACATGATCTG |
| | ATTTAGGTTTATAAAAGATCTCTTGAATACCTCTTGATTATAAATGGGCAAGAGTA |
| SEQ ID NO: 637 | ATTCTTTGAGATATACATTTAATATGTTTGATATGATTTTGGCTGTTAAATTGTATGATGTGT |
| | CTTCATTTTATGAACCATTAAAAAAGAAAAAAACATGCTATAAATATAACTGAAAGAAGCC |
| | ATTTGTTGCAAGATATTCTGATGTCAGAGATCTTATGAAATTTGAACACATTTTAGAACTGC |
| | AAAAATACAGTTAAGTAGAATAGTCAATGAAAAAAAGAAATCTATTTGCATATATAAATGT |
| | CCTATGAAATTTGATTTTCCAGTGAACACTTTACAAAAACAAATAAAGAATAT |
| SEQ ID NO: 638 | ACGTGTATTGTTGTTAATATGGACACATGACATATTTGTCTGCCTGACTTTTGATCCCAGCTA |
| | CAACCTCTGGCCTTTTCAAATGATTCTTTAATATCACATAAAGGGAGGTGAGATAACTAAAG |
| | GGGGTGATTCCGGACTAGGAGGCAAGGGAGGGCAGATAACATGGTGCTTTGAAATCTTCTG |
| | TGACTTTTCAGTCACTTATTTCATTAAGTGATATATCTCACTAGAAGTGAGGTAGAACATAA |
| | CAAATCCATGTTTGCTGGGCATATGTTATGACAACAGAGAAATTCACAGACT |
| SEQ ID NO: 639 | GACCATTTCATAATGATAAAGGGCTTATCTCACCAAGAAGGCAGTCACACTTACGTTTTTAT |
| | GTATTTGTTGAAGAGTCCCAATGTATTTAAAGCAAAAATAAGCAACTACAAAGAGAAAGAT |
| | ACAAATCCATGATCAAAGTGAGGAATTTTCACACACATCGTAGTAACTGATGGAATGAGTC |
| | AATGAAAAATTAGTGAGGAAATAGAAGATTTGGACAGCACAACAAATGGCCTAGGAGAAC |
| | ATTTAGAATGTTGCCTTCGATGCTTAAGAATACATATTCTTTTCAAAAGAAAACAC |
| SEQ ID NO: 640 | TCTCGATCTCTTGACCTCGTGATCCGCCCACCTCGGCCTCCCAAAGTGTTGGGATTACAGGC |
| | GTGAGCCACTGCGCCCAGCCAAGATCCCAGTTTTTAATAAAAAACTTTTCTCATAGATAAAC |
| | TTGAAATAATTTTGAGGACAAGGTAAAACTGTCATTTGTTTTCCATGCTGCCTAGGAGGCTT |
| | GTAGTATTTATTGAATACAAGTGGAATTCATTTGACGTATGCATCAGAATTAGTTTAAAAAT |
| | TGGGATTGTCTTTCTTAGACAAATTCAGAGGTCATCCAAAGATAAAGGAGAG |
| SEQ ID NO: 641 | ACTGCAACCTCCACCTCCCAGGTTCAGGCGATTATCCTGCCTCAGCCTCCTGAGTAGCTAGG |
| | ATTACAGCCCAGCTAATTTTTTGTATTTTTAGTAGAGATGGGGTTTTGCCATGTTGGCCAAG |
| | CTGGTCTTGAACTCGTGACCTCAGGTGATCCACCTGCCTCGGCCTCCCAAAGTGCTGGGATT |
| | ACAGGTGTGAGCCACTGCACCCAGCCTCCCTGATTATTTTTTAATTAAAAAATTAATGCAAA |
| | TATCATGAACAATAAAGAAAGACTAAGAAACTCTCTTAGAAGGAAACTAAGG |
| SEQ ID NO: 642 | AGGAGGATATATAACTCATGTCTATAAGGCCTTGAGGTCCTGTGCTAGATGGCATGCTATGT |
| | TTTGATCAGATTTAATTGAGAACTAGCTCTGATATTTTATTTATGTATTTATTTATTTATTGTA |
| | GAGATGCGTAGTCTTGCCATCTTGCCCAGGCTGGTCTTGAACTCCTGGGCTCAAGCAATCCT |
| | CCCACTTCAGCCTCCCAAAGTGCTGGGATTATAGGTGTGAGGCCCCATGCCTGGCCCTACAT |
| | ATTCTAAAATAAGAAAAGGTGTTCTGCTATTTAGAAATGACTGCCAAATC |
| SEQ ID NO: 643 | CTCAGCCTCCCAAAGTGCTGGATTACAGGCGTGAGCCACCGCGCCTGGCCTAATTTTGTATT |
| | TTTAGTAGAGACAGGGTTTTTCCATGTTGGTCAGGCTGGTCTCGAACTCCTGACCTCAGGTG |
| | ATCCGCCTGCCTGGGCCTCCCAAAGTGCTGGGATTACAGGCGTGAGCCACTATGCCCAGCC |
| | CCTAGTCAGCAATTTTCTTATCGGGCATAATTTAGATGCTTATAAACCCAAGGGGTATACCC |
| | ATCCTTAGAACTACAAAGACCCATCACACCTGCTGTCTAAATGTTTCCACATA |
| SEQ ID NO: 644 | GAGTCTTAAGAAGCTGACATTTTAAAAACAAAAAACTCAAGTTGTTATTATAGTATTGTGAC |
| | ACTTCTGCATAATTTCCTAACAAAGACCAGCATTCATAGTGCTGAGGAACAGACTTTTCCAG |
| | GCATTTTACTGAATAAGCTGTAAAATGCTGCCTTTGACTTTGCTGGTGGCCTGCTTGGCATA |
| | GCTTCTCTGGGCCTCTGCTTAACTTTCCCAGACAGCACAGCACTTTGAAACACTCTCCCCAC |
| | TGAATACAACTAACAAGTTCTTGATAAATCTCTACTATCAAAACTTATTTGG |
| SEQ ID NO: 645 | AGGATTCCTGGGGCCACCATAACAAAGCACCACAAACTGAGTGACTTAAAACAGTAGCAGT |
| | TTCTTCCCTCCCAGTTGTGGAGCCCAGAGTCTGAAGTCAAGAAGTCCTCAGGGCCACACTCC |
| | TCCTCCTCCTCAGGATAGAGGGGAGAATCCTTCCTCTTCTAGTTTCTGGTGGCTCCTGGCATT |
| | TCTTGGCTTATGGCAGTTTGACTCCAGTCTCTGCCTGTGTCTTCACGTAGCCTTTTTCAATGT |
| | CCCTCTGTGTCTATATCCAAATCTCCGTCTCTTTCATGAAGACACCATTTT |
| SEQ ID NO: 646 | ACTCTCAGAACCTTGGTTAACCTCAAGAAACATTCCAGTTCCCTATCAGATAGGAATCCAAG |
| | ACTCCCTCTGTGGACACCAGGCACCATGAAATCCAAGTCTGGCAATCCAGAGGTGAGACCA |
| | CAGTTTCAGTTTCTAGGGTCTAAATTGATTAATGCAAATGTAATCCATTTACAGTATAATCC |
| | CAGGTTCATTGCCCTGTGGTAATTCAAACAATGTGAGCATTCAAGGCACAGAAATAACAAA |
| | AAGGCTTTTTTTAACTTCTAGTTATTTTTAAACATTTTTTTTTAATCATCATAG |
| SEQ ID NO: 647 | CAACTACAGATGTCACTGAATTCCCATTACCTAGAACCGTGCCTGACAAACAAAAGATACT |
| | TGAAAAAATTAACAAACAACCCTACAAGGTATGTCATATAACCATTTGCCAAGAGAAAAGA |
| | GAAGATTAGATTTCAGAGACAAATAACTTACCTAAAGCTGCATAGTAAATAAATAGCAAAT |
| | GCACCTGAAAGAGCCAGCACTCTACCCACTGACTCACTACAACTTGCGACATAAAGCATAT |
| | ATGCTAAGAAACAAAAACGCAAAAGCAAGCATTTCTACATGAAAACACCAGATCAC |
| SEQ ID NO: 648 | GACTTGCTCTTAGCTGCTTTGTGAAACTGAGGATGTGCATTAGGTGTGGAGTCAATCTGCTC |
| | TCTTTGAAATGAGATTCAGAACAAAAAAACAACTAAAACCTAGCAAAAAACCAACCACCA |
| | ACCAACGAACTGCCTGGGAGTTCAGCTATCTGCCTGTCTGATCTCAGGTCTGAAAGCCAGAC |
| | CATCACCCCTGGCTCTCTCTCTCCCAACAGACACTGCTGGAGAGTATCTGAATCCAGACCTT |
| | TTGGAAAACAACATAAAGAGATATTAAATATTTTGACCTCAAGAAATCCCAACT |
| SEQ ID NO: 649 | TGCTAGGTCATATGGTAACTTTGGAGAAACACCATTTTATGCTCCTCCTGGCAATGTATAGG |
| | GGTTCCAATTTCTTCACATTTTTGCTAATACTTGTAATTGTCTGTCTTTCATATTTTTGTCATT |
| | CTCGTGAGTGTGAAGTGGTATCTCATTGTGGTTTTGATTTGCATTTCCCTAATGACTAATGGT |
| | GTTGAATATCTTTTCATATGCTTATAAGCCATTTATATGTCTTTGGAGAAATTCTTTTCAAAT |
| | CTCTTGCTCATTTTAAAATTAGGTTGTCATTTTATTACGGAGTTGCAT |
| SEQ ID NO: 650 | ATATTGATCCAAGTGAGATTTCAATGTGGAAGAGAGGGTCCTACAGCTGAAGAAAACTGGG |
| | GAGTCACTCTTCCAGAGCAAGCTCTTCCAGCACCTCCTCTGCCTTCCCATAGTCCACAGGCT |
| | GCCTCCGGATCTCTACCATAGCAGTGAACCTAGTGAATTACCGTGACTCATTTCTCTGTCAA |
| | TTTTCCCCCTAAACCATGAGTTCCTCAAGGAAAGGACCATGTTGGATTTGTTGCTGTATCTCT |
| | GGCTCCCAGCACAAAGGAGGCGGTTGTGCATGTGAGATGAAGGAGGGAGAAC |
| SEQ ID NO: 651 | TGGCTGGGAGATGACCCAGGTGGGTCTCTATAGGGTGGCATGGCAGCGAGCACAGACATGG |
| | CTGGGCTGGAGGAGAGCTTCCGCAAGTTTGCCATCCATGGTGACCCCAAGGCCAGTGGGCA |
| | AGAGATGAATGGCAAGAACTGGGCCAAGCTGTGCAAGGACTGCAAGGTGGCTGACGGAAA |
| | GTCCGTGACAGGGACCGATGTGGACATCGTCTTCTCCAAAGTCAAGTGAGCCCTAAGCAGC |
| | CCCTGCTTCTCATGTTCCCAGGAGGTGGGGAACTGGGGGGCTGGAACCAGGGAGGAC |
| SEQ ID NO: 652 | ATTGTTCATGTTGTATGCAGAAGCAGAATGTCAGAACTACTCTCGCCATTCTCCTGCCTCAG |
| | CCTCTCCAGTAGCTGGGACTACAGGTGCCCACCACCATGCCTGGCTAATTTTTTGTATTTTTA |
| | GTAGAGATAGAGTTTCACCGTGTTAGCCAGGATGGGTGAACCCAGGAGGCAGAGCTTGCAG |
| | TGAGGCAAGATAGCGCCACTGCACTCCAGCCTGGGCGACAGAGTGAGACTCTCTCTCAAAA |
| | AAATAAATAAATAAAAGAACTAAGTACTTTCTGCCTGATGAAACTCAGAGTTG |
| SEQ ID NO: 653 | TGCCCAAGGTCACAGACTGGAAGATAGTAAGATGTGAACTCAAGAAACTGGGATCAGAGC |
| | CCACACTCTCACCCAATACCCTACGCAGTCTCCCTTGAAAAGATAAGGCTGTTCTTAACTAC |
| | ATGATCTAAAGTGGCCAGTCAGTATGCAAGTGCACCATAGATCTGGAAGGGATCACAGAGA |
| | AAACGGGAAGAAAGGGATTTCTTCTGGAAAGTGGAAGTGAAAATTATATAACATTTTATTC |
| | TCCATGGAAGCTTTGTCTGAATAATGTCATATGCCTTACTACTAAGAAAGGTGCAT |
| SEQ ID NO: 654 | GAGAACTTAAATTCTATGCTTCTCTTGTTGTCAGATAGAGTTTGTCAGCTGTTTATGGTTAGG |
| | AGCTATGTGGCAACCATTGATTTTTGTAATCCCCAGCACCTAGCAAGTGCTTAATACTGTTT |
| | AGATGTTTCGTTAACAATCAATTGGTAAAATAGCATGACATAAGTACCTTTAAGTCCAAGTT |
| | ATTCTACTTTCTGCTTTGTAGACATTAAATCCATGGGTTAATAGGATTGTTTCATCCTGGTAT |
| | AGAGAAAAGACAAATTTAAGGTGTCTTTGCTTGTCTTAAAAACCAAGTTT |
| SEQ ID NO: 655 | AGCTGCTTTGCTAGCGTGCAGATGGAGAAGGCGGGCCCGGGAGGCTTGGAGTTTGGCCCAG |
| | GAGCTGGCAGGTGCCCAGCAGCAGTTTCACTGCCCGCTTCAGGCGACTCTCCACTGGCTTCC |
| | TGGGAGCCTTCCGTTAGGAGATGCTCTCTCAAGATCCCCGTCACCAGGGCTTTCTGGTTGGA |
| | ATGAGGCAAGGACCTGCTTTACACGGTGCGCCCCATTACCCAACAGCGTTTCCTGTCCTTGC |
| | TGGCCACAGCCATCACAGTGGGGCTGTCATCAGCCACTTTAAACAGGCAAAAC |
| SEQ ID NO: 656 | ATGAACTGATAATATACTACAGCTACAAAAAAAAGGTGGCGGGGGGGAGGATAGTGATAA |
| | AGAGCTAAAAGCACCTTTGTGTGAAAAATAAATTATCAGAAGAGTATTTGCCAAACGCCTA |
| | GCTACTATCACTCTGATTAGCACACAATTCGGTTAGGCATTTTCTGAGGAAAATTCCTATGC |
| | CCAAAGATCTTTAAATGAGTCACGATTCTCATCTGAAAAAAAAAAAAATTAGAATGCTGAC |
| | AACACTAAGGTAAATTCAACTTCATAAAAGCATTTCTTAAAAACTGTGTTTCTAAA |
| SEQ ID NO: 657 | TACAAAAATTAGCCGGGCATGATGGCGGGTGCCTGTAATCCTACCTACTCGGGAGGCTGAG |
| | GCAGGAGAATTGCTTTAACCGGGGAGGTGGAGGTTGTAGTGAGCCGAGATCACGCCATTGC |
| | ACTCTAGCCTGGGCGACAGAGTGGGACTCCATCTCAAAAAGAAAAAAAAAGGAAAAAAAA |
| | GAAACCTCAGCAGAGGAGGAAAAACTGTAAAAAAAAGAAAAAAAAAGAATTTTGTGCATA |
| | GAAATTGAAAATTTGGAAATGAAAATTTCAGACTAAAAATTAAAATGTCAAAGAAATC |
| SEQ ID NO: 658 | GCCCCGGCTCCCCCAGGCAGAGGCGGCCCCGGGGGCGGAGTCAACGGCGGAGGCCACGCC |
| | CTCTGTGAAAGGGCGGGGCATGCAAATTCGAAATGAAAGCCCGGGAACGCCGGAAGAAGC |
| | ACGGGTGTAAGATTTCCCTTTTCAAAGGCAGAGAATAAGAAATCAGCCCGAGAGTGTAAGG |
| | GCGTCAATAGCGCTGTGGACGAGACAGAGGGAATGGGGCAAGGAGCGAGGCTGGGGCTCT |
| | CACCGCGACTTGAATGTGGATGAGAGTGGGACGGTGACGGCGGGCGCGAAGGCGAGCGC |
| SEQ ID NO: 659 | CTTTCAGTTTGCTGAGAATGAGGCCTTCCAGCATCATCCACGTCCCGCAGAGCGCCCTCTGT |
| | GAAAGGGCGGGGCATGCAAATTGGAAATGAAAGCCCGGGAACGCCGGAAGAAGCGCGGGT |
| | GTAAGATTTCCCTTTTCAAAAGGCGGAAGAATAAGGAAATCAGCCCGAGAGTGTAAGGGCG |
| | TCAATAGCGCTGTGGACGAGACAGAGGGAATGGGGCAAAGGAGCGAGGCTGGGGCTCTCA |
| | CCGCGACTTGAATGTGGATGAGAGTGGGACGGTGACGGCGGGCGCGAAGGCGAGCGC |
| SEQ ID NO: 660 | CGGCTCCCCCAGGCAGAAGGCGGCCCCGGGGGGCGGAGTCAACGGCGGAGGCCACGCCCT |
| | CTGTGAAAGGGGCGGGGGCATGCAAATTGGAAATGAAAGCCCGGGAACGCCGGAAGAAGC |
| | ACGGGTGTAAGATTTCCCTTTTCAAAGGCGGAGAATAAGAAATCAGCCCGAGAGTGTAAGG |
| | GCGTCAATAGCGCTGTGGACGAGACAGAGGGAATGGGGCAAGGAGCGAGGCTGGGGCTCT |
| | CACCGCGACTTGAATGTGGATGAGAGTGGGACGGTGACGGCGGGCGCGAAGGCGAGCGC |
| SEQ ID NO: 661 | GCCCCGGCTCCCCCAGGCAGAGGCGGCCCCGGGGGCGGAGTCAACGGCGGAGGCCACGCC |
| | CTCTGTGAAAGGGCGGGGCATGCAAATTCGAAATGAAAGCCCGGGAACGCCGGAAGAAGC |
| | ACGGGTGTAAGATTTCCCTTTTCAAAGGCGGAGAATAAGAAATCAGCCCGAGAGTGTAAGG |
| | GCGTCAATAGCGCTGTGGACGAGACAGAGGGAATGGGGCAAGGAGCGAGGCTGGGGCTCT |
| | CACCGCGACTTGAATGTGGATGAGAGTGGGACGGTGACGGCGGGCGCGAAGGCGAGCGC |
| SEQ ID NO: 662 | ACAAAACCCTCTGCCGGGCTCTTTGGGGGCGGAGTCAACGGCGGAGGCCCACCGCCCTCTG |
| | TGAAAGGGCGGGGGCATGCAAATTCGAAAATGAAAGCCCCGGGAACGCCGGAAGAAGCAC |
| | GGGTGTAAGATTTCCCTTTTCAAAAGGCGGAGAATAAGAAATCAGCCCGAGAGTGTAAGGG |
| | CGTCAATAGCGCTGTGGACGAGACAGAGGGAATGGGGCAAGGAGCGAGGCTGGGGCTCTC |
| | ACCGCGACTTGAATGTGGATGAGAGTGGGACGGTGACGGCGGGCGCGAAGGCGAGCGC |
| SEQ ID NO: 663 | GAGCCCCGGCTCCCCCAGGCAGAGGCGGCCCCGGGGGCGGAGTCAACGGCGGAGGCCACG |
| | CCCTCTGTGAAAGGGCGGGGCATGCAAATTCGAAATGAAAGCCCGGGAACGCCGGAAGAA |
| | GCACGGGTGTAGATTTCCCTTTTCAAGGCGGAGAATAAGAAATCAGCCCGAGAGTGTAAGG |
| | GCGTCAATAGCGCTGTGGACGAGACAGAGGGAATGGGGCAAGGAGCGAGGCTGGGGCTCT |
| | CACCGCGACTTGAATGTGGATGAGAGTGGGACGGTGACGGCGGGCGCGAAGGCGAGCGC |
| SEQ ID NO: 664 | GAGCCCCGGCTCCCCCAGGCAGAGGCGCGCCCGGGGGCGGAGTCAACGCGGAGCCACGCC |
| | CTCTGTGAAAGGGCGGGGCATGCAAATTCGAAATGAAAGCCCGGGAACGCCGGAAGAAGC |
| | ACGGGTGTAAGATTTCCCTTTTCAAAGGCGGAGAATAAGAAATCAGCCCGAGAGTGTAAGG |
| | GCGTCAATAGCGCTGTGGACGAGACAGAGGGAATGGGGCAAGGAGCGAGGCTGGGGCTCT |
| | CACCGCGACTTGAATGTGGATGAGAGTGGGACGGTGACGGCGGGCGCGAAGGCGAGCGC |
| SEQ ID NO: 665 | CTCGAGCTCCTGAGTCGAGACGGGATTTCTCCGTGTTTGCCAGAATGGTCTTGATCTCCTGA |
| | CCTTGTGATCCACCCGCCTCGGCCTCCCAAAGTGTTAGGATTACTGGCGTGAGCCACTGCGC |
| | CCGGCAGATTTTTCTTTTAAAACGTGGAGAATAAGAAATCAGCCCGAGTGTGTAATGGCGT |
| | CAATATTGGTGTGGACAAGACAAAGTGAATGAGGCAAGGAGCGAGGCTGGGGCTCTCACC |
| | GCGTCTTGAATGTAGATGAGAGTGGGACGGTGATGGCAGGGAGGAAGGCGACGAC |
| SEQ ID NO: 666 | TCCATGTTGGTCAGGCTGGTCTTGAACTCCTGACCTCAGGTGATCCGCCCGCCTCGGCCTCC |
| | CAAAGTGCTGGGATTACAGGCATGAGCCACCGCGCCCAGCCAAGAAATAACAATATTTGTT |
| | ATTTTGACTTTTTTAAAATGTGTATCACCTTGTTCTTTTTCTTTCTCTCCTTGAATATTTAAAC |
| | ATCTTCACTAACTGAACTAAGAAATTGAACTAACTCAACTATAAAACCGAGAGAGGGGGGT |
| | AAGTAAATAGTCTTTTCATGGGAACATGACAGAAAAACAGAAATAAGACATC |
| SEQ ID NO: 667 | AACATGGAGAAGCCCCATCTCTACTAAAAATACAGAATTAGCTGGGTGTGATGGCGCATTC |
| | CTGGAATCCCAGCTACTTGGGCGGCTGAGGCAGGAGAACTGCTTGAATCCGGGAGGCAGAG |
| | GTTGCGGTGAGCTGAGATCGCGCCATTGCACTCCAGCCTGGGCAACAAGAGCAAAACACCA |
| | TCTCAAAAGGAAAAAAAAAAAAAGAAGAGATAGATTTGGCTTCAAATAACAGGAAAACAC |
| | AAAATAATAGTGGCTTCAGTAAGACAGGACATTATTTCTCTTTCTCATAAACACTCA |
| SEQ ID NO: 668 | ATGAGGAGCATGGCCACGATGGCCGGCCTCATGTGTCTGGCTCACTTCTCTCAGGATTCCTA |
| | AAAGGGATTTCTCACATCAGCTTCCATTGGTCTTTCATTTCTGTGGGGCAGCAGGGATTATA |
| | CTCTTCTCTCCTGCCCCTAAACAATGTAACGAGACACTGTGGAGTGCTGTCTGTGGACACAC |
| | AGGAGGTGGGCCTGGAACCCAGTGCTCCTGCCTCCAGCTCCAGATAAAGGAATGCACCTTC |
| | GACTGTCTCCTCTAGGACCCCAGTGTAGCGGGAATGCCTTAAAACAAAGTTGT |
| SEQ ID NO: 669 | AAGGAACTGCTGGTTGGAAGCGGGCCGAGATGAGAACGCAGTTGCGCTCTCTGACCAGCCA |
| | GGCCTATGCATGACGTCACGCCGGGAGGTGGAGTATGTAGATTAAAGACTGCATTTTGGAA |
| | ACGCGTTCCTTGGAAGGATTTGCACAACTCTGTTACCAACACCAAGATATAGTATAAAAAA |
| | TCTGTTTATTTTGTTCACTATATGTGGATAAAGTCCAATTAGAGTCATTTCAGGAGTTACCCG |
| | CACTTGCAATGATGTGGGCGGCACCGGGGATTGCTGGGGTCACGCAAGTACCTC |
| SEQ ID NO: 670 | GCTGGGCTTACAGGCACCCGCCACCATGCCAGGCTAATTTTTGTATTTTTAGTAGAGATGGG |
| | GTTTCACCATGTTGGCCAGGCTGGTCTTGAACTCCTGACCTCGTGATCTGCCTGCCTTGGCCT |
| | CCCAAAGTGTTGGGATTACAGGCGTGAGCCACCGTGCCCAGCCATTTCTGGCTCTTAAACTT |
| | TGGAAAAGTCTTTATCTTGTAAGTAGCAGGCTAGAACTTGTGCAAGAAGAGTGACCCTATG |
| | TGAAGAGTGTTTTGAATCTGAGGGTGAGATGTCAGGCAACAACCATGCCTTC |
| SEQ ID NO: 671 | TTAAGTTTTATTTCAGTCGTGTCACCAAGCATACTAGGAAAGGACATACTGAATGAGATCAT |
| | CCAAAGGGGTGTGGAGTATGTAGATAAGCGCAGTGACTGCTGGCTATTTGCACCCTAGGCT |
| | TATATTTCTTAAGATTTTGAATAGCCAGATATTAAAAATACAGGTGCTGGGTTATTGTAGTT |
| | TGGAAAATATCTTTATTCCAGAAATTAGGGGGTCTAAAGAGCACATGAGAAAAATGATCCT |
| | ATATTTAGAGTGGTTTGAATCTAAGGGTGAACTCTCAGGCAGCGCTGGGGACTC |
| SEQ ID NO: 672 | CTCAGCCTCCCGAGTAGCTGGGATTACAGGCGCAGGCTACCATGCTCGGCTAATTTTTGTAT |
| | TTTTTAGTAGAGATGGGGTTTCACCATGTTAGGCTGGTCTCAAACTCCTGACCTCAAGTGAT |
| | CCACCCGCCTCAGCCTCCTAAAGTGCTGGGATTAGTCGTGAGCCACCACACCTGGCCCTTTC |
| | CTTCCTTTTCTTCCTTTCTTTCCTCACTTCCCTTCCATTCCTTCATTCCTTCTCTTTCCTTCCCC |
| | TGCCTTCCCTTCCTTCCTTTCTTTCTTTTGTCTTAAAGATTCCTGTGAT |
| SEQ ID NO: 673 | TACTTTCTGCTGATAATATTAATATTTCTGGAATGCTATCTGGCAATTTAAATACAAAGGTC |
| | CAACAATGTTCATATCCTTTTATCCCTTCAGTTTTATGTCTGGGATGTTATTTTAAGGAAATA |
| | GTCAGAAATCTATTTTTAATTAACGTAAAAAGATGTTTCTTATCATTATTTATAACAGTACAC |
| | AATTTAAAACAAAATTTAAAAAATTTAAAATTTAAAACAAAATAGGGACATAGTTAAATAA |
| | ATGATTATGTTTCCATATGATGAAATAGTATGTGTTAAAAATAACATTTAC |
| SEQ ID NO: 674 | CAGGCGCCTGCCACCATGCCCAGCTATTTTTTTGCATTTTTAGTAGAGATGGGGTTTCAGCC |
| | TGTTATCCAGGATGGTCTCGATCTTCTGACCTCGTGATCCACCCACCTCGGCCTCCCAAAGT |
| | GCTGGGGTTACAGGCGTGAGCCACCGTGCCCGGCCCACACCTGGCTAATTTTTGCGTTTTTA |
| | GTAGAGACAGGGTTTCATCATGTTTGCCAGGCTGATCTCAAACTCCTGTCCTCAAGTGATCC |
| | GCCTGCCTTGGCCTCCCAAAGTGTTGGGATTACAGGCGTGAGCCACCACACC |
| SEQ ID NO: 675 | ATTAGACAAATTAACATTATTGATATAGCAGTTTGCCATATTGAAAAGTATGAAGATTTGAA |
| | TCAAAGTTACCAGCTATAAAATTTGGACAATTGTGTACAAAATAGAAATAACAATACCTAG |
| | CTTACAATACAGTTGAACAAGACGGTATCTGTAAAGCATGACGTGTATATAGTGCATAGCA |
| | TGTTAACAAGTTTCAGTTCCCTCCTCTCCATTCGCCTCAAATCTATCAGGAGCGCTAATCTCT |
| | AACCATCTCTATGCTCTGACACAAAGTTCCCTGTGAAAAACATTATACAAAGT |
| SEQ ID NO: 676 | ATCAGCAAAAGGTTAAGGTAACTGAACAGGACAAATGACCCAGTTTCTCTAACAAATCAAT |
| | GGCATGAAAGGAAACAAAAGGAAAGGGAGAATGTTAGAGAATAAAGGAGACCCAAGAAA |
| | CATAATAACCAAATACAAATGGCTTTATTTGAAGCAGGATCCAAGCAACTTATGAAAGACA |
| | TTTTTTAGACAAGTGAGAAAACATGAACGTGAACTGTGTATTAGATGATCATCAGGCATCA |
| | CTAGTACTTTTGTTAGGTGGGATAACAACATGGCGGCTGTGTTTAAAAAGGAGAAAGG |
| SEQ ID NO: 677 | GGAGAAACTCATAACAAATGTCAAGTAGAAAAGAGGGTTGTGAAGCTCTTAGGATGTTGTC |
| | AGAAGATGAATAGGATTTTTACAATGACCGATGTTTCAGAAAAGGAAAAACAATATTGATG |
| | ATTCTTAGAGATAAATTTATAGCTAGAGTAAGAAGTCATGAATCTGTAGTTCAGGAAACTTG |
| | AAATAATAGGTGACACAGAACACATAGTTTATCTCCTTTTTATCTTCTCATTTCTGAAACAA |
| | GAAGTTGCTCTGTAAATTAGAAAAACTAGAACAAACAAAAAGAAAATAGACAAT |
| SEQ ID NO: 678 | CCTCTACCGTGAGTCTCCTCTTCCTCTCTACTGAAATCCCAAACATTTCTTGAAATAGTGGCT |
| | CTTATACCTTACTATGCATAAGAAAAACCCTGGAGCATCTGAAAGAGTTCTGTTTCCAGCTC |
| | TCCCTGCCCTAGAGGTTCTGCTTCACTGGGGCTGGGATAGGATGTGCACAGGACATTTTTTT |
| | GTAAGCTGTCCAGTCAACTCCAAGACAATCATGGCTCTGGTCTGAAAACGACTGCTTTCCGC |
| | TTTTCTCTGCACAACCTTCCCCAAATCCAGCTCTCAGAATTAATTGCTCTC |
| SEQ ID NO: 679 | ATGAGGTACCAAGACACTGAATAATGGGAATGTAATAATTTGGAACACAGATGCTGCTACA |
| | GGTCAGGGCTTTCATCCTATCCTGGTATAAAAGTCATACCGCTTTCTAAGAAATCTTTTTACT |
| | TTTAAATAATATACATATTGCTTGATTTAGATACCCAATCAAAACACATTCTTTCCAAGTAT |
| | GAAATTTAATTTGTATACATATGGAATAAAGATAAATAGAAAATCCATTTTGCTAATATACG |
| | TAATAGTTGCATGTAGCTGACCAAAGCATTCACATGTGGGGCCTAAACATTT |
| SEQ ID NO: 680 | GTAATACGGCAACATTGTATTCACAGAAGTACAGAATACATAGGAATACTGTCTCTACAGT |
| | CTTGGGCCTTTCTGCTGGCAACTGTGACACTGTGACACACACATTTGCTTTTTATTACATCAA |
| | AATGTGATGTGTTCTCTATTCTCAACCCCAGACAGCCTGCATTTCTCACTAGGTCCTGCCAAT |
| | TTGTATTCTTCCAAAACCACCCAATTCTTTAGACAGATCCTTTCTCTCTAGAGGTGTGTTCAT |
| | TAGCTCTGCAGAACGTATAGTCTCCATTCATTAAAATTTAAAAAAAAACT |
| SEQ ID NO: 681 | AACATCTTCACCATCATGTTTATCAACCTCAGAGTATTCTATGGTAAGAATGTTTATTTATTC |
| | AATTCCTATAATCATATTTAGGATATATATATTATTATTATTAAAAAGTGGACTGCTGTGAA |
| | CATGTTTGCCAGATTTTTATGCATTATCATAAATGCACAATTTATCCTTTTCTTAAAGATAAA |
| | TTTCTAGAGGTAGAATTTCTAGATCAAAGAACATACAGGTTTTTTTAATAGCTTTTAAAATA |
| | GATAAAGTGGCCAAGTTTCTATCAATTTTACGTTTGTGTATTAAAACGCA |
| SEQ ID NO: 682 | GCAGGCGGATCACCTGAGCTCAGGAGTTCGAGACCAGCCTGACCAACATGGAGAAACCCCA |
| | TCTCAACCCTGTCTCTACTAAAAATATAAAATTAGCCGGGCATGGTGGCGCATGCCTGTAAT |
| | CCCAGCTACTCGGGAGGCTAAGGCAGGAGAATCTCTTGAACCCAGGAGATGGAGGTTGCCG |
| | TGACCTGAGATCGCGCCATTGCACTCCAGCCTGGGCAACAAGAGCGAAACGCCATCTCAAA |
| | ACAAAAAGAAAAGAAAACTACAAAACAAAGAATCAAAGATATAAATAAATGAAGA |
| SEQ ID NO: 683 | TCTCCTGGGTTCAAGCAGTTCTCCTGCCTCAGCCTCCCGAGCAGCTGGGATTACAGGCGCCC |
| | GCCACCACGCCCAGCTAATTTTTGTATTTTTAGTAGAGACGAGGTTTCACCATGTTGGCCAG |
| | ACTGGTCTTGGACTCCTGACCTCAGGTGATCCACCTGCCTCCCAAAGTGCTGGGATTACAGG |
| | CGTGGGCCACCACGCCCAGCCAATTACATGATTTATCTCATGTGTTAGAGGTGATAACTATA |
| | TGGAAAAGAATAAAAGAGGGAAGGGAGCCTTGTGATTTTAAGTAAAGAGGTC |
| SEQ ID NO: 684 | TGTGTGTGTGTGTGTGTGTGTGTGTGTGTGTGTGTGTTTTAAGTCCCTAGAAAATAATGCAG |
| | CACTCTAGTCCTCCCAGTTGTCCTCTCAAATCTATTTTCCATCTTCCTGGATACATGGCTGCC |
| | TAACCAGTGGCTATATTTCCCAGCCTCTAATATTGGGCTAATGAGAAGTAGGCAGGAGTGA |
| | ATGACCTGAGCCACTTCCGGCTATTTTGCTTTTGCCCTTTCTCAGGTGCATCAATGTGGAGAC |
| | GGCAAGGACAGCAGCAGCAACAGCACCTGGGAGCTCGTTAGAAAGGGAAAT |
| SEQ ID NO: 685 | ATCTCATGATGTATATGCAATATTCCAAAACTCAAAAAAATCTGAATCCAAAGCACTTCTGG |
| | TCCCATGCATTTCTGATAAGGGATATTCAACCTGCATCAGTTTTCCTGTTTGTTTCCTTTGAC |
| | TTCTCTTCCACACTGCTGGTTTCATCATATGTCTGGTCGGTCCTCCCTGTCTGTTATGGAAAG |
| | GATTCTGTGATTTGTGTGGATTTCCTATCCTTCTGTGTAGGAAGGCCTGTTTCCTGGGGATAG |
| | GTGGAAGAAGGGGGTTTGTCCAGGGCTTACAGATAAGATTTCACTCTAG |
| SEQ ID NO: 686 | GGGATTCACCAGGGCACAAGGCGACGTGCGTCTGTCCTCACGGAGCCTAAGTGCTGGTGGA |
| | GACGGACAGTGCAGATGCCAACCGCAAATGACGAGGCACTTGCAGGTAGGGGTCAGCGCC |
| | AAGAAGGAAATGAAGGGTGCGTGTCGCTGACTGAGACTCTGGTGGGGGCTACTTAAATAGG |
| | GCCATGGGGGAGGGCCTCTCTGAGGAGGTGACATTTGAGCTGAGGCCTCACCAATCAGAAG |
| | GAAAAAGCTGTAAAGGGTCTGGGATTAACTCAACCTGGGGACTTTTAAAATAATGTA |
| SEQ ID NO: 687 | GGGATTCACCAGGGCACAAGGCGACGTGCGTCTGTCCTCACGGAGCCTAAGTGCTGGTGGA |
| | GACGGACAGTGCAGATGCCAACCGCAAATGACGAGGCACTTGCAGGTAGGGGTCAGCGCC |
| | AAGAAGGAAATGAAGGGTGCGTGTCGCTGACTGAGGCTCTGGTGGGGGCTACTTAAATAGG |
| | GCCAAGGGGGAGGGCTTCTCTGAGGAGGTGACATTTGAGCTGAGGCCTCACCAATCAGAAG |
| | GAAAAAGCTGTAAAGGGTCTGGGATTAACTCAACCTGGGGACTTTTAAAATAATGTA |
| SEQ ID NO: 688 | GTCAGGAGTTTGAGACCAGCGTGGCCAACATGGTGAAATCCCGTCTCTACTAAATACACAA |
| | AAATTAGCCGAGCATGGTGGCACACGCCTGTAATCCCAGCTACTTGGGAGGCTGAGGCAGG |
| | AGAATCGCTTGAACCTGAGAGGCAGAGGTTGCAGTAAGCCGAGATCGCACCACTGCACTCT |
| | AGCCTGGGCGACAGAGGGAGACTCCATCTCAACAGAAAAAAAAAAAAAAAGAACATCCCA |
| | CAGCAGAAGTTCGTTCATTTATTCATTCATTTATTTTATTGTTCAACAACTACATCT |
| SEQ ID NO: 689 | AAACATCCTCAAAGATTAAGAAAAGGCACTGCAAATATCAGATCAATTATGAAAACGATGT |
| | TCTGATTAGATGTCATTTGAATTGCACTATTATTCACCAAAGGATATTGTAGGCAAGCATTT |
| | GTAATAGGGGAGGAAATGATTTGGAATTGCTAAAGATTAGGAGGGCTTGAAAACAAGCCTT |
| | TATTGGCCTTTGAAGCCTTGGGAAGAATGTTTGCTCTTCAGGTGCCCGTGTAGCCCTGCTCT |
| | GGAGATCTTCTCAGATGCTGTCCTACTGCATTGTCCAAATTAAACAGAGAAGTC |
| SEQ ID NO: 690 | GGTTTCACCTTGTTAGCCAGGATGGTCTCGATCTCCTGACCTCATGATCCACCCGCCTCGGC |
| | CTCCCAAAGTGCTGGGATTACAGGCGTGAGCCACCGCGCCCGGCCAATACGGCAGTTTTAA |
| | GTAGGAATTTACTAAGTTCAAAGCGAACTAATGTGTCTCCTAAAATTCAAGTCCTATATGGT |
| | GTCAGCATTTTCATTGTAATTGATAAAGTATTAACCAGCCATTTGAACATCTATTATATGAT |
| | ATGCACTGAGCTCAAGTCTAGATACACAAATGAGTAATAAATGAACCCTTCCA |
| SEQ ID NO: 691 | TGTCTGTGTTTTATATCATTTTTCTTCTATTTGTGTTTGTCTCTGTGTCTAAATTTTCTTTTTAT |
| | AAAAGGACATCATTCGTACTAAAGTAGGGCCCACTCTAATGACGTCATTTTAAGTTGCTCAT |
| | TGCAAAGATCCTATTCCCCAGTAAGGTCACATTCACAGGTACTTAGGGTTAGGATTTAAACA |
| | ACTATATATACATAGCTGGAAAATATTGACCCCTCCATTGAGGGATAGATAGATAGATAGA |
| | TAGATAGATAGATAGATAGATAGATACTCAAAAGTATATATATATGCTTT |
| SEQ ID NO: 692 | ATACTATACATAAACATTGATTGAGCTCGGGGGGACTTTCTGTATATACATTTAAAAATATG |
| | TTTTTTTTAATAGAGACAGGGTCTCACTGTGTTGCCCAGGCTAGTCTCAAACTCCTGGGCTC |
| | AAATTATCCTCCCCACTTGGCCTCCCAAAAGGATGGGATTACAGGCATGAGCCACTGCCCA |
| | AAGGCTAAAATTTTTTTAAGTACCATTAGAATGTAAGGATTCTTTTTAAAAAATTTGATTGT |
| | GCAGGGTTGGTTATTCAACCAATATGCAATACAATATTCAATACTGTATATTC |
| SEQ ID NO: 693 | TGTGGCTTTGGCTCCTGTCCCTGTGAGGGTGTTAAAATAACATCCTCAAAGTGAGTATCTGG |
| | TCTGGTGGCCTCACTTCCTGTTCATGATGGCTGAGTTCTTGGTTTCTGGCACCTGGAAGTGTC |
| | CTTTTCCTGTGTAAGCACAGCTTCACTTTTTACACTTAAAAAAAATTTTATAGAGTATTTTAG |
| | TTAGGTGTTGGGGCAACAGGGTGAGGGTGGTTGGAGGTGCTTTCCCACATCAGCTGGGTTC |
| | ACCCTCTTGACTGGCGATCAGTAGTTTTACTTTGTATTAGAAACAAAACCC |
| SEQ ID NO: 694 | GAGTAGGGAGGAGAAGAGGTGGGGACAGGAAATGGAAAGTAGCAAGAAAAGGGGAAATG |
| | TAGAAGAGAGGGAAGGGGGAGGGAGATGGGGAGAGGGAGGAGGGAAGAGGCATGGCAAG |
| | AGGAAGTGGGAAAGGGAGTTGGGGAGGATAAGTCACAGACTGAGCAACCACAGTTCCTCA |
| | CACTGTGCTTGATTATTTCCGTAGAGGTCAATCCATTTACTTCTTATAATCTGTGAGCTGAGT |
| | CAGGAAGCTTGCAACTCAGTAAGGCAGAAGCCTTAGATTCACACAGCCAATAATATTGG |
| SEQ ID NO: 695 | TTCCCACAAAGGTAACATCTTGCAAAAGAATAGTATAAGATCTCAACCAGGATAGTCAATC |
| | CACTCATCTGATTTAGATTCCCCAAGTTTTACTTCTCCTTGTGTGTGTGTGTGTGTGTGTGTG |
| | TGTGTCCTCTCCAGCTACAAATGTCATGTTCCTTATTCAGGATAATCTGTGGTTAAATTCAGG |
| | ACTTAGAGAGAGAAGGAGGTAGGTTTATGCAAATATCAGATTTCTTTGATGGTAGGCAACA |
| | AATGGACACAGGCTAACTTAAGGAAAAAGGAAATCTATAAAAGCTACGTAGG |
| SEQ ID NO: 696 | CCCACATAACATAATATTTACTACCTTAAACATTGTTAAGTGTATAATTTAGTAGTGTTAAG |
| | TATATTTACGTTGTCATGAAATAGGATCTCCAAAACTTTTTCATCTTGCAAAACTGAAACTC |
| | TATACCCCTTAAACAACAACTCCCCACTTCCCAACCAACTCCTGACAAACAACATTCTACAT |
| | TTTGTTTCTGTGAATTTGACTACTCTACATATCTTATATAAGTGGAATCATACAGTCCTGTTT |
| | TTTTGGTGACTGGTTTCTCTTTGGTAATTGATTCTTTAAAAAGAATCAGGC |
| SEQ ID NO: 697 | AAAGAACTTACTCATGTAACTAAACACCACCTGTTCCCCAAAAACCTGTGGAAATAAATTTT |
| | TTTTAAATAAAGGAATTTGAGACCCCCCGCCAAAAAAAACCAAAAAACCCAAAAGTACATG |
| | ATGGCTTTGGAAAATAGGCAAGAGGGTTAATGAATTAAAGGCCTTGAGGCAGTTTAAGGGT |
| | AAATTCAGGGAGTATAAGGTTTAAGAGAAGTAGAACAACAAGTAGTTCTGAGAGTGGACCC |
| | TCTGTGTTTGGTTTTGGAAGTAGAGCCATTCCAAGTGTCACTCCAGCAACAAATA |
| SEQ ID NO: 698 | ACAGCTAGGGAGAAGATCAACAAGGAAATAGATTTGGACAACGTGATAAACCAACTAGAG |
| | CAGAATATACATTCTTTTCAAGTGCACGTGGAACATTTTCTAGGACAGAACATAAGTTAAAT |
| | CACAAAAGAAGTCTCAGTAAATTTAAAAGGATTGAAATCATACAAAGTACTTTGGAATTAA |
| | ATTAGATGGAAAGGAATGAAATTAGAAATCAATAAAAGAAGGAGATTTGGGTAATTCATAA |
| | ATATGTGGAAATTAAACAGCACACTCCTAATACAAATAGGTTGAAGAAGAAATCAC |
| SEQ ID NO: 699 | CAAATATTTGGGAACCAAATAATGTGCTTTCAATTACCCTATGAATCAAATAAGAAATCATA |
| | ATGAAAATTAGAAAGTGTTTTGAACTGACTGAAAAGGAAAACACAACATATCAAAATTTGT |
| | GAGAGGCTGGTTAAAAAAAAAAAATGACTCGGGAAATTTCGTAGCAGTAAAAACACCTCTA |
| | TGAAGAAAGGTCTCAAGTCAATGAGCTCAGAAAACATGTTAATAAATGAGAAAAAGGAAA |
| | ACAAATTAAATTAAACAGAACAAAGGTAATGGTAAGTGTCAGAGTAGAAATAATAC |
| SEQ ID NO: 700 | CTTTTTTTATTATGGCAGAGTACATGTAACATATAGTTTGCTATTCCAACTGATTTTTGACAA |
| | AGATACAACAGCAAATCAATGGAGGAACAATAGCCTTTTTAACAAATGGTGTTGGCACAAC |
| | TGGACAACTGTAAGCAAAAGAAAATGAACTTCAATCTAAATCTCACACCGTATTAAAAAAA |
| | ACTCAAAGTGGGCCACAGACTTAGATATAAAATGTAAAACTATAACACTTTTAGAAAAAAT |
| | ATAGGAGAAAATCTATGGGATTTAGGGCAAAAGCATGATTCAAAAAAGGAAAGT |
| SEQ ID NO: 701 | TTTGAGATGGAGTCTCACTCTGTCGCCCAGGCTGGAGTGCAGTGGTGCGATCTCGGCTCACT |
| | GCAAGCTCTGCCTCCCAGGTTCAAGCCATTCTCCTGCCTCAGCCTCCCAAGTAGCTGGGACT |
| | ACAGGCGCCCGGCTAATTTTTTGTATTTTTAGTAGAGATGGGGTTTCACCATGTTAGCCAGG |
| | ATGGTCTCGATCTCCTGACCTCGTGATCCACCTGCCTCAGTCTCCCAAAGTGCTGGGATTAC |
| | AGGTTTGAGCCACCGCGCCCGGCCAGGGCAGGGTATCTCTCTTAAAACCTGG |
| SEQ ID NO: 702 | ATGTCTGCTTCCCCCTTTCCACCACGATTATAAGTTTCCTGAGGCCTTTCCAGTCACGCTAAA |
| | CTGTAAGTCCATTAAATCTCTTTCCTTTACAAATTACCCAGTTAGCAGCATGAGAATGGACT |
| | AATACACTTAGCTATACTAAGGAGATACACTGGTGAACAAAACAGACAAAACTCCCTTCCC |
| | TCACTGACTTTACATTATACTGTTGTAGAGTTTCTCTCAAACCTGATGATGGCTTTCCAATTC |
| | CGATCGAAACAGAAAATAAGGTAATGAACTCTGGATTAAGAATCATCTGAG |
| SEQ ID NO: 703 | CTTTTTTTTTTAACCTAACACTGTATCTTGGAAATGACTTCATATTAGTCCACAAAGAGCTTC |
| | TTTGTTTTTTATTCTATTTCATTTTGCCAGCCCCCTATTGAAGGAAACATATTGCTTCCAGTCT |
| | TTTGCTATTATCTAACAATGCTGCAATGAATAACATTGAACATATGTCAGATAAATTCCTAG |
| | GGGTGGAATTACTGGGTCAAAAGGTATATATATTTATTATGTTTGATAGTGACTTCCAAATT |
| | AAACTGAAAATTTTGTAATTTTTTTTCCACTCTAAAGAAATAGAAGTTC |
| SEQ ID NO: 704 | CAGAGCGAAAATGACTGTTAGATACAATAGAACACTCTAAGAGTCATTATCAAAAATGAGC |
| | ATATCGGTGTGTAAAATGTATGGTAGTTTTATTTAACGGTTGTTTCATTGTTTACCGTAGTGT |
| | TTTTTCTTACAATTTTGTGGAAGCCTGTGTCAGAGTTAAGAACTTTTATAGAAGAGGATAAT |
| | CATGGATGATTGAAATTGACATTTTAAGCTGATACTGAAAGTTATTCTAACTTCTATTACATT |
| | TATAGTTGTATTTTCTTTCAAAGGATAATGGAAGTCTTAAAAAGAAAATGG |
| SEQ ID NO: 705 | CGCAACACCTTCTTATTTCACGACGTATGGTCGTAAAGCAATAAAGATCCAGGCTCGGGAA |
| | AATGACGGAGAGGTGGAACTATAGAGAATAAATTTGCATATATAATAATCCGCTCGCTAAT |
| | TGTGTTTCTGTTTTCCTTTGCTAAGGTAGAAACAAAAGAATAATCACAGAATCTCAGTGGGA |
| | CTTTGAAAATATCCAGGATTTTATACGTGAAGAATGGATGTATCGCATTACGGTAGTCACCC |
| | TATGTGTAAATTAGTGGCACATACTTGGCACTCCTTAATGTCAACTATAAGATG |
| SEQ ID NO: 706 | TCTATTTTGGTTACTACGGTAGGTGCCTGGCGGAAGGGAGTGGGCGGAGATATGTAAATAG |
| | AAAGTGCGTACAGTTAGAACGTCCGGCACGTAACTGATCGGAGCATTCTGGGAAGAAGTAA |
| | TTTATTCCTTTTCGGCAGCCGAATGAAAAAAAAATTTAAAAAAATGTGCGAGCTAATATGG |
| | CAAAACAACTGGAAGGACGCTAAATAATAGGATTGCTCATACCAGCGCGTTATGAACTCAC |
| | CCTAGCTTGTAACGGAATCTTTTTCACTGAGTGCAGAATGTCGGCTGTTTGTCTGT |
| SEQ ID NO: 707 | TCCGCGGACCAACTCTCGCGACAGCCAGCTCAAAGCAGGCAAGAACCGGAAGGGGGGGG |
| | ACGTTCCCCGTGAGCCTTCGCGGTGCTGGCTGCTCATCTGCATACGGAAGTTCGGCACATTA |
| | TGAATTATTTATTTTCCTCGAGGGAAAAAATTAAATGAAAAGCAACAAAATACATTATTAA |
| | CAAGTGAGACAAACTTCAATGGAACTGGATCATGACCTCAACAGTCAACTACGATAGTCAT |
| | CATACGCCTAATGAGAATAGAATTCATTACCTAGGAAATAAACTAAAAACGTCCTT |

In some embodiments, a promoter sequence may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263. In some embodiments, the promoter sequence comprises a sequence of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263. In some embodiments, the promoter sequence is selected from SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263. In some embodiments, a PSE of a promoter sequence of any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263 is replaced with a PSE of any of SEQ ID NO: 31-SEQ ID NO: 37 or SEQ ID NO: 67-SEQ ID NO: 120. In some embodiments, a PSE of any of SEQ ID NO: 31-SEQ ID NO: 37 or SEQ ID NO: 67-SEQ ID NO: 120 is inserted or substituted into a promoter of any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263. In some embodiments a PSE sequence is extracted from any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263 and inserted into a different promoter (e.g., any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263). In some embodiments, the PSE of a promoter of any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263 is replaced with a PSE extracted from a different promoter (e.g., any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263) or is replaced with a PSE of any of SEQ ID NO: 31-SEQ ID NO: 37 or SEQ ID NO: 67-SEQ ID NO: 120.

Promoters of the present disclosure may have insertions or deletions of nucleotides on either side of the promoter. Nucleotide bases may be inserted or deleted between the promoter and the 5′ ITR or between the promoter and the payload. In some embodiments, a promoter sequence of the present disclosure (e.g., any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263) may be truncated by 1 to 2, 1 to 3, 1 to 5, 1 to 10, or 1 to 20 nucleotide bases from the 5′ end, the 3′ end, or both the 5′ end and the 3′ end. In some embodiments, a promoter (e.g., any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263) may be truncated by 1, 2, 3, 4, 5, 6, 7, 8, 9, 10, 11, 12, 13, 14, 15, 16, 17, 18, 19, or 20 nucleotides from the 5′ end, the 3′ end, or both the 5′ end and the 3′ end. In some embodiments, 1 to 2, 1 to 3, 1 to 5, 1 to 10, or 1 to 20 nucleotide bases may be added to the 5′ end, the 3′ end, or both the 5′ end and the 3′ end of a promoter sequence of the present disclosure (e.g., any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263). In some embodiments, 1, 2, 3, 4, 5, 6, 7, 8, 9, 10, 11, 12, 13, 14, 15, 16, 17, 18, 19, or 20 nucleotides may be added to the 5′ end, the 3′ end, or both the 5′ end and the 3′ end of a promoter (e.g., any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263). The nucleotides being added to the 5′ end or the 3′ end of the promoter may be selected from any nucleotide (e.g., A, T, C, or G). For example, SEQ ID NO: 1250 comprises an 18-nucleotide base truncation of the 5′ end of SEQ ID NO: 376. In another example, SEQ ID NO: 1251 comprises a 2-nucleotide base truncation of the 5′ end and a 2-nucleotide base addition to the 3′ end of SEQ ID NO: 168.

A promoter (e.g., any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263) may have nucleotide additions on the 5′ end in order to extend the expression cassette. In some embodiments, a promoter (e.g., any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263) may have additional nucleotides added to the 5′ end in order to extend the promoter to a total length of 200 nucleotides, 300 nucleotides, 400 nucleotides, or 500 nucleotides long. For example, SEQ ID NO: 1262 is an extended version of SEQ ID NO: 1250 with an additional 118 nucleotides added to the 5′ end to extend to a total length of 400 nucleotides. In another example, SEQ ID NO: 1263 is an extended version of SEQ ID NO: 1251 with an additional 100 nucleotides added to the 5′ end to extend to a total length of 400 nucleotides.

### Termination Sequences

An expression cassette may comprise a termination sequence (also called a terminator). A termination sequence may be an endogenous termination sequence. A termination sequence may be an engineered termination sequence engineered to increase expression of an RNA payload. Examples of endogenous termination sequences (e.g., SEQ ID NO: 1243), engineered termination sequences (e.g., SEQ ID NO: 60, SEQ ID NO: 1242, SEQ ID NO: 1256, SEQ ID NO: 1257, SEQ ID NO: 1275, or SEQ ID NO: 1287-SEQ ID NO: 1289), and additional termination sequences (e.g., SEQ ID NO: 771, SEQ ID NO: 930, SEQ ID NO: 1002, SEQ ID NO: 1007, SEQ ID NO: 1017, SEQ ID NO: 1021, SEQ ID NO: 1244-SEQ ID NO: 1247, SEQ ID NO: 1254, SEQ ID NO: 1255, or SEQ ID NO: 1264-SEQ ID NO: 1272) are provided in TABLE 6.

TABLE 6
Exemplary Termination Sequences

| SEQ ID NO: | Sequence |
|---|---|
| SEQ ID NO: 60 | CCCAATTTCACTGGTTTCAAAAACAGAAAAACAGTTCTCTTCCCCGCT |
| | CCCCGGTGTGTGAGAGGGGCTTTGATCCTTCTCTGGTTTCCTAGGAAA |
| | CGCGTATGTG |
| SEQ ID NO: 771 | CTATTTAAAAAAAAAAAAAAAAAAAAGGAAGAAGAAAGGAAAAAA |
| | AAACGGTTAAGGCCAGGAGGAAGCATCTCAGTGGCAGGTCTTAGCCT |
| | CATGCCCA |
| SEQ ID NO: 930 | CTGAAATAAAAAAAAAGAAAAACAAAGCTCAAGTTTGCCTCATTTGG |
| | CAAATGATTCCCAGGTGAAAGCTAGCGTTCCATGTTCTGCCTACTACT |
| | CTCTA |
| SEQ ID NO: 1002 | AATTTTTGTAATGAAAAAATAGACGGCAAGGGTTATTCTTAAAACTG |
| | CAGTTTTGTAGCTTGGGTGGCATGTTAAGTGTTCTCCTTACAGTCGCA |
| | ACGAT |
| SEQ ID NO: 1007 | AATTTTTGTAATGAAAAAATAGACTCCCCTATAAGGGTTATTCTTAAA |
| | ACTGCAGTTTTGTGGCTTGGGTGGCATGTTAAGTGTTCTCCTTACAGT |
| | CGCA |
| SEQ ID NO: 1017 | ATCATGTTTTATAAAAAAAGACTTAAAGAGGAAAACATTATGGTGCA |
| | ACTTTAGGCTTAAGTGATTCATTGTCACTGTTTGTTTAAACATTGTGT |
| | AACAG |
| SEQ ID NO: 1021 | GTCACTCTTGTCCAATGAGAGATCATAACTTGAAGTCGGTGGTCTTTA |
| | TTGTATAATTTATTTATTATAAAAATGCATACAACATAAAAGCATCTT |
| | CAGC |
| SEQ ID NO: 1242 | CCCAATTTCACTGGTTTCAAAAACAGAAAAACAGTTCTCTTCCCCG |
| SEQ ID NO: 1243 | CCCAATTTCACTGGTCTACAATGAAAGCAAAACAGTTCTCTTCCCCGC |
| | TCCCCGGTGTGTGAGAGGGGCTTTGATCCTTCTCTGGTTTCCTAGGAA |
| | ACGCGTATGTG |
| SEQ ID NO: 1244 | CCCAATTTCACTGGTCTACAATGAAAGCAAAACAGTTCTCTTCCCCGC |
| | TCCCCGGTGTGTGAGAGGGGCTTTGATCCTTCTC |
| SEQ ID NO: 1245 | CCCAATTTCACTGGTCTACAATGAAAGCAAAACAGTTCTCTTCCCCGC |
| | TCCCCGGTG |
| SEQ ID NO: 1246 | CCCAATTTCACTGGTCTACAATGAAAGCAAAACAGTTCTCTTCCCCG |
| SEQ ID NO: 1247 | CCCAATT |
| SEQ ID NO: 1254 | TATAAAGCTGTTAAAAAATCAGATTGACTTCATTTAGGGTGTTTCTTA |
| | CAGATATCGTTTAAGTTTTCGGTTCTGCTTGTAAACGCTTCAATCGC |
| SEQ ID NO: 1255 | ATTTTAAGTGTTTTAAAAACAGATGCGATTCCGTTAAATCGCGTGTGG |
| | AGCTATGTAAAGTGTATTATAGAACAAATGCGAGTTACGGTTTTTCA |
| | GCTTT |
| SEQ ID NO: 1256 | ATTTTAAGTGTTTCAAAAACAGATGCGATTCCGTTAAATCGCGTGTGG |
| | AGCTATGTAAAGTGTATTATAGAACAAATGCGAGTTACGGTTTTTCA |
| | GCTTT |
| SEQ ID NO: 1257 | GAATGTTCTGTTGCCAATGATAGACGTGTGGGTGGGGTGTTTCATGCT |
| | TTGGGAGGTTGGGGTAGCTCCACAAATGTCACCCAGGTTGTAGCGGA |
| | GTGGT |
| SEQ ID NO: 1264 | AATTTTTGTAATGAAAAAATAGACGGCAAGGGTTATTCTTAAAACTG |
| | CAGTTTTGTAGCTTGGGTGGCATGTTAAGTGTTCTCCTTACAGTCGCA |
| | ACGATGGGAAACAGAAAGTAACGTGTTATCCTCTCCGCCGCCGTGAG |
| | CTCTTTTAACACTAGCTAAGTGGCCGCAGGGCTCTTCTCTTTCCTTTCC |
| | ACTTGGGGC |
| SEQ ID NO: 1265 | ATCATGTTTTATAAAAAAAGACTTAAAGAGGAAAACATTATGGTGCA |
| | ACTTTAGGCTTAAGTGATTCATTGTCACTGTTTGTTTAAACATTGTGT |
| | AACAGAACTTGCAAAGACAGTTAACTCTTGTTTTCCATGTCAAAGGT |
| | CTGAATACTTGCATGATAAAAGTCTGTGTAACTTTCCCTGGTGACATC |
| | TGACTTGCTA |
| SEQ ID NO: 1266 | TCTAAGTAAGTTAAAAGTAGACTTTGGGTATTTACCGAGATCTCTGCA |
| | AACACAGAACTTCTGTTCTCAAGTGTATCATTTTATATCACTAGCTGT |
| | TAAA |
| SEQ ID NO: 1267 | CCTTAAGTTTAAAAAAAGGTATCTGTGCTCTCAAGGCTTTAAACTTTG |
| | TGTTTAAAAGTTTTAGAGCCTTGAGAGCACTTCTCTAAAACTAAAAAT |
| | TGTT |
| SEQ ID NO: 1268 | CCTTTAGAAGTTAAAAAACAGACGTTAAAACTTGTAAATTCTAGTAT |
| | CAGTAGCTTTAAAACACAAACAAAAAATACACTAGAAAAATACAGC |
| | AAGATTA |
| SEQ ID NO: 1269 | CTTAGTAAGTTTAAAAACAGAAAAAAAACCGTGTTGCTACAGCTATA |
| | AACTTCAAACATGCAGTTTATAGCAGTGGGCAACACGTCTCATCTCA |
| | AAAATT |
| SEQ ID NO: 1270 | CCTAAGTCAGTACAAAAACAGAAAGTCCGCGCTCTTACTGCTTGATA |
| | CTTCAACAAGAAGTTACAGCAGTGAGAGCGCTGCTACATTATTTAGA |
| | ACTTCC |
| SEQ ID NO: 1271 | CTGTTACTAGTTTAAAAACAGAAGTTGCTACTCGTTAAAAAGTACTA |
| | AACAAACAAGCTTTTTAAAACTTAGCTTTAAAAAATCAACAATAATT |
| | TTGAAC |
| SEQ ID NO: 1272 | CTTCCGTAAGTAAAAAACAGAACTGTGCTTTAAACTGTTTTTAACAG |
| | AAACGCCTTGCGTCAAAATGAAAGTTCTTAAGTAAAAGCGCTCGTAT |
| | CAAAAT |
| SEQ ID NO: 1275 | CCCAATTTCACTGGTTTCAAAAACAGAAAAACAGTTCTCGTTTCAAA |
| | AACAGATTCCCCGCTCCCCGGTGTGTGAGAGGGGCTTTGATCCTTCTC |
| | TGGTTTCCTAGGAAACGCGTATGTG |
| SEQ ID NO: 1287 | CAATTTCACTGGTTTCAAAAACAGAAAAACAGTTCTCTTCCCCGCTCC |
| | CCGGTGTGTGAGAGGGGCTTTGATCCTTCTCTGGTTTCCTAGGAAACG |
| | CGTATGTG |
| SEQ ID NO: 1288 | ATTTCACTGGTTTCAAAAACAGAAAAACAGTTCTCTTCCCCGCTCCCC |
| | GGTGTGTGAGAGGGGCTTTGATCCTTCTCTGGTTTCCTAGGAAACGCG |
| | TATGTG |
| SEQ ID NO: 1289 | TTCACTGGTTTCAAAAACAGAAAAACAGTTCTCTTCCCCGCTCCCCGG |
| | TGTGTGAGAGGGGCTTTGATCCTTCTCTGGTTTCCTAGGAAACGCGTA |
| | TGTG |

In some embodiments, an expression cassette comprises an engineered termination sequence (e.g., SEQ ID NO: 60, SEQ ID NO: 1242, SEQ ID NO: 1256, SEQ ID NO: 1257, SEQ ID NO: 1275, or SEQ ID NO: 1287-SEQ ID NO: 1289). The engineered termination sequence may enhance expression of a payload (e.g., a small RNA payload) encoded by the expression cassette. In some embodiments, the engineered termination sequence may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 60, SEQ ID NO: 1242, SEQ ID NO: 1256, SEQ ID NO: 1257, SEQ ID NO: 1275, or SEQ ID NO: 1287-SEQ ID NO: 1289.

In some embodiments, an expression cassette comprises a termination sequence that may enhance expression of a payload (e.g., a small RNA payload) encoded by the expression cassette. In some embodiments, the termination sequence may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 60, SEQ ID NO: 771, SEQ ID NO: 930, SEQ ID NO: 1002, SEQ ID NO: 1007, SEQ ID NO: 1017, SEQ ID NO: 1021, SEQ ID NO: 1242, SEQ ID NO: 1243-SEQ ID NO: 1247, SEQ ID NO: 1254, SEQ ID NO: 1255, SEQ ID NO: 1256, SEQ ID NO: 1257, SEQ ID NO: 1264-SEQ ID NO: 1272, SEQ ID NO: 1275, or SEQ ID NO: 1287-SEQ ID NO: 1289.

In some embodiments, a 3′ box sequence element that may be included in an engineered termination sequence may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to any one of SEQ ID NO: 40-SEQ ID NO: 42 or SEQ ID NO: 121-SEQ ID NO: 166. In some embodiments, the termination sequence comprises a sequence of SEQ ID NO: 60, SEQ ID NO: 771, SEQ ID NO: 930, SEQ ID NO: 1002, SEQ ID NO: 1007, SEQ ID NO: 1017, SEQ ID NO: 1021, SEQ ID NO: 1242, SEQ ID NO: 1243-SEQ ID NO: 1247, SEQ ID NO: 1254, SEQ ID NO: 1255, SEQ ID NO: 1256, SEQ ID NO: 1257, SEQ ID NO: 1264-SEQ ID NO: 1272, SEQ ID NO: 1275, or SEQ ID NO: 1287-SEQ ID NO: 1289. In some embodiments, the termination sequence is selected from SEQ ID NO: 60, SEQ ID NO: 771, SEQ ID NO: 930, SEQ ID NO: 1002, SEQ ID NO: 1007, SEQ ID NO: 1017, SEQ ID NO: 1021, SEQ ID NO: 1242, SEQ ID NO: 1243-SEQ ID NO: 1247, SEQ ID NO: 1254, SEQ ID NO: 1255, SEQ ID NO: 1256, SEQ ID NO: 1257, SEQ ID NO: 1264-SEQ ID NO: 1272, SEQ ID NO: 1275, or SEQ ID NO: 1287-SEQ ID NO: 1289.

In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 60. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 771. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 930. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1002. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1007. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1017. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1021. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1242. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1254. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1255. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1257. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1264. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1265. In some embodiments, a termination sequence for enhanced expression of an RNA payload may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1269.

In some embodiments, a termination sequence, also referred to as a terminator, may enhance transcription of an RNA payload. The termination sequence may be positioned downstream of the payload sequence. Additional exemplary termination sequences of the present disclosure are provided in TABLE 7.

TABLE 7
Additional Exemplary Termination Sequences

| SEQ ID NO | Sequence |
|---|---|
| SEQ ID NO: 708 | ACATTTGAATTTTTTCTTAAAAAAAAAAAAAAAAAAAAAAAAAAAAAAGGAGACACACC |
| | ATCTTCCATGGACTGAATATCTGTGTTTCTCCAGAATCCAT |
| SEQ ID NO: 709 | TATTTTAAGTGTTTTAAAAACAGATGCGATTCCGTTAAATCGCGTGTGGAGCTATGTAAAG |
| | TGTATTATAGAACAAATGCGAGTTACGGTTTTTCAGCTT |
| SEQ ID NO: 710 | ACTTTCTGGAGTTTCAAAAACAGACCGTACGCCAAGGGTCATGTCTTTTTTCGTATTGGTTT |
| | GTGTCTTAGTTGTTAATCCTACAGTGGAGGCCTGGGGA |
| SEQ ID NO: 711 | ACCTGACATAACGGGGTTCAAGACTGACAACGCCTCACGCCCACCCGAAAACGTTTACAT |
| | GGCTTCCTTGTCTCTTTTTTTTTCTGTCCTAAAGTCGCCT |
| SEQ ID NO: 712 | ACTTTCTGGAGTTTCTAAAAGTAGACTGTACGCTAAGGGTCATATCTTTTTTTGTTTTGGTT |
| | TGTGTCTTGGTTGGCGTCTTAAATGTTAATCCTACAGT |
| SEQ ID NO: 713 | GTTTCACTCTTGTTGCCCAGGCTGGAGTGCAATGGTGCAATCCCGGCTCACTGCAACCTCC |
| | ACCTCCCGGTTCAAGCGATTCTCCTGCCTCAGCCTCCTG |
| SEQ ID NO: 714 | TTTATAAATAAAAAAAATTTTATAATGATGCATCTTTACAAAGCTAACATGTTTAGTTTAAC |
| | AATTTTATTAAACATCACTCAAAGGGTGAGTCAAATGT |
| SEQ ID NO: 715 | TCTTTACTGTTATATGTTAGGCGAAATATTACGCGTTTGGAGTAAGTGGTGCTTTTTGTAAC |
| | TGAAAAGAGATTCTGTGTGTGTTTTTTTTTTTTTTTAG |
| SEQ ID NO: 716 | CTTCCATCTCAAGAAGCTGCCAGCCTGGGCAAAATGGTAAAACCCCATCTCTACAAAAAA |
| | ATAAAAAAAATTAGCTGGCATGGTGGCACATGCCTGTGGT |
| SEQ ID NO: 717 | CCTCCGCCTCCCGGGTTCAAGCAATTCTCCTGCCTCAGCCTCCCAAGTAGCTGGGGTTACA |
| | GGCATGCGCCACCACACCTGGGTAATTTTTGTATTTTTT |
| SEQ ID NO: 718 | CCTCACTAAAACAACAACAACAACAACAAAAGACAAAATTTGAATTCCAGAAATAGTTAT |
| | TGCAGCAACAAGTCAGTCTCCCTGCTGGAAACACCTATCA |
| SEQ ID NO: 719 | AAGAAAAACCCATTTTCCACAAACACAAAATCTCAACAATGAGAAAACAAACAACTCAAT |
| | AAAAAACGGGCTAAAGCTCTGAACCAACACTTCGCCAAAG |
| SEQ ID NO: 720 | AAAAAAAAATTCTTTTAAAAAATTAAAAAGTGAAAATGCAGTTACTATAAAGGAATGACA |
| | ATTTTGTTTGCAGCAGATTAAATGAAACAAGAACTTGCAT |
| SEQ ID NO: 721 | TGGTTAAAAAAAAAAGAAAAAAGAAAGCCTATGTGAATGTCTGCATATGATCCTTGAAAT |
| | CGGGGATCCAATTACTTTTGTGATTCCCAGAACACAGGCA |
| SEQ ID NO: 722 | GGAGGTTGTAGTGCAAAAAGCAGTTTGTCTACCAAGTGATACTTTCAGCTTTTACAAATGC |
| | TGAACAATATCCGTGGTGTGTTTTCATGTCACCTCCTCT |
| SEQ ID NO: 723 | AGAAAATTTCCACATATTTCAACATGTATCTTGCAAAAAGCCTATGAACTTCAATTTGTATT |
| | CATACTTGTAAAATTATGTTCACACATTATTTCTTGAG |
| SEQ ID NO: 724 | GGTTGTTGTCGTTTTAAAAGTGGATTTTGTTTGCTGTGGAAGCATATACAAGGCGTTAGTAT |
| | AAACTAATAAACAGGTCTTTGTTCCCATCTGTAACTTA |
| SEQ ID NO: 725 | GTGCTTCTGTGGTGCGAATAGTAGGTGAGCCGTAAGTGTTTTTGTAACTCAGGGTGCGGGC |
| | TCGTGTTTTGTGGCTGTGTTCTGTCCGGTCAGTTGTTTC |
| SEQ ID NO: 726 | CCTAACATAACGGGGTTCAAAACTGACATCGCCTCACGCCTACCCGAAAACGTTTACGTGG |
| | CTTCCTTGTCTCTTTGTTTTTTCTGTCCTAAAGTCGCCT |
| SEQ ID NO: 727 | GGTGCTTCTGTGGTGCGAATAGTAGGTGAGCCGTAAGTGTTTTTGTAATTCAGGGTGCGGG |
| | CTCGTGTTTTGTGGCTGTGTTCTGTCCGGTCAGTTGTTT |
| SEQ ID NO: 728 | ATTTTTGTAGTTTAAAGAACAGTCTGCACGGCGAGGGTTACTTGTTTTTTTTACTGGCTTGT |
| | GCTTTACTCTTAATCGTTTCTCTCACAGTCGGAGGTTG |
| SEQ ID NO: 729 | GGAGGTTGTAGTGCAAAAAGCAGTTTGTGTACCAAGTGATACTTTCAGCTTTTACAAATGC |
| | TGAGCAATATCCATGGTGTGTTTTCATGTCACCTCCTCT |
| SEQ ID NO: 730 | GGTGTTCGTGCTACCCCGGGGCTGGGTTCGCGCACGCTCCTGACCTGCCTTGGCTCACGGC |
| | CAACGCGGATATCGCCGCCAGAGACCCTTCGCCGCCCTC |
| SEQ ID NO: 731 | AGATACTGCAGTGCAAAACGTGAGCTCTACGCGAAGGGCTACTCTTAGTTTCTATGGGTTT |
| | GTAGCTTTGACGGCATGTTAAGTGTCGTCCTTACTGTCT |
| SEQ ID NO: 732 | GGATTGCTGTCGTTTTAAAAGTGGATTTTGTTTGCTGTGGAAGCATATACAACGCGTTAGT |
| | ATAAACTAATAAACAGGTCTTTGTTCCCATCTGTCACTT |
| SEQ ID NO: 733 | GCGCTTTTGTGGTGCGAATAGTAGATGAGCCGTAAGTGTTTTTGTAATTCAGGGTGCGGGC |
| | TCGTGTTTTGTGGCTGTGTTCTGTCCGGTCAGTTGTTTC |
| SEQ ID NO: 734 | GGTTGTTGTCGTTCTAAAAGTGGATTTTGTTTGCTGTGGAAGCATATATAAGGCGTTAGTAT |
| | AAACTAATAAACAGGTCTTTGTTCCCATCTGTAACTTA |
| SEQ ID NO: 735 | CAGGGGATTAAAATTTAATGTTCAAAACAGAAGAAGGAAAAAAAAACAGTCTGTCTATGT |
| | GGGATGGGATACAAAAGAGTGGGATAAGAATTATCTTTTG |
| SEQ ID NO: 736 | CCCCCACAAAAAGAAAAAAATGCATTCTAAAAAAAAACTAAAGAATGCTTTAGGAAGTAT |
| | TGACTGGCAGTAGTGTACAGCATGAATAGGATCAAGGCAA |
| SEQ ID NO: 737 | CTGAACTTAAAAAACAAAAGTTGATTGAAAAAAAATTTTTTTTTTTTTTTGAGATGGAGTCT |
| | TGCTGTCGCCCAGGCTGGAGGGCAGTGGCACGATCTTG |
| SEQ ID NO: 738 | AAGTAAATTAAAAAAAAAAAAACTATTAACAGAGTAAACAGACAACATAAAGAATGAGA |
| | GAAAATATTTGCAAACTGTGCATCTGAGAAGGGTCTAATAT |
| SEQ ID NO: 739 | ACTTAGACAATGCTGAGAAATGAGAAGGTATGGCTGGCTTGGCTTTTAAGCTGAAACTTTT |
| | ACTACAGGCCTGGGAGTTATGACAGCTGGAGTCTGAATA |
| SEQ ID NO: 740 | TTTTTTGTCTGTTTGTTTTTAGGTCCTGCTTGGGCAGGGCCAAGTAAAAAAGAAAAAAGAA |
| | AAAAGAAAAAAACCACAAAAGGTCATCGAATTAGGGTCA |
| SEQ ID NO: 741 | CATGCAAAAAATTATATTATTATGTAATTATTATTAACTAATGTCCAATTTTCACAAAACAT |
| | GTTTAAAAATTTTCTCTAGACATTTGTCAGGGAGTAGT |
| SEQ ID NO: 742 | CCCCCCACGGGATATGGGATATGGAGAGACAACTGTAACTTGTTTCCATATCTGCTTATCT |
| | CAACCAGAGGACCTTGTACAGTACCAGAACCCAATAGGT |
| SEQ ID NO: 743 | CCCTGAAAAAAAAATTAATTTAAAGTTTAAAAATATTTTAATAAAATAAAATAAGTCCAG |
| | ACTTACCTGAGAAGACATAGCAAGGACCTGTTCAAGATAA |
| SEQ ID NO: 744 | GGATGGAAAATGATTGTGTAATTGGTACGAGGTTTCCTTTGTGATGATTAAAATGTTCTGG |
| | AACTAGATAGTGGTGTCAGTTACACAATATTGTGAATGC |
| SEQ ID NO: 745 | GTGTTAGATGATGTTAAGCACTAGGCAGAATAATAAATTTGGGAAGAGGTAGAGGAAAAA |
| | TGTAGGTTACAGTATGGGCTAAGTTGTTGTAACAAAGTGT |
| SEQ ID NO: 746 | ATCCAGCACTCCCCAGTAGTCCTTTCATAAAAGAGCCCTGCCATTCCCAAGTCCATTGTCA |
| | TGATTTATTGCTTGCTTGTTGGGATTTCACCATCTAGAG |
| SEQ ID NO: 747 | CGGGGGAAAAAAAAAAAAATGAAAGAAGCACATGTCTTATTCATATGTAAAATGCCTAGA |
| | GTAACAGACACTCAATAGTCTATTTAGGTATAGATTACTC |
| SEQ ID NO: 748 | AATAAAAAGTTTTTTAAAAAAACTTTATTTAGGGCAGGCGCAGTGGCTCATGCCTGTAATC |
| | CCAGCACTTTGGGAGGCTGAGGCAGGTGGATCCCCTGAG |
| SEQ ID NO: 749 | GCCTGCAAATGTTTGATAGATACTCTAAAATTACTCTCTAGTGAGGCTGTACCAATCAACA |
| | CCTCATCTCAGCAGTGCGTTGATGCTCCTGTTTCCCCAA |
| SEQ ID NO: 750 | TGTGTGTGTGTTTGTGTGTCTAGTAGTGTTTACATGCATTCTTCCCTTGGGGATATTTTTTTT |
| | TTTTTTTTTTTGAGACGGAGTTTTTCTCTTGTTGCCC |
| SEQ ID NO: 751 | AATGATTAAAAAAAAAAGTCAAACACATTATTGAAAGAAGCAAGTTAAAATTGAAGTTAA |
| | AAACTGAATATGTGGGCTGGGCATGGTGGCTCACACCTGT |
| SEQ ID NO: 752 | CCACATCCATTTCCTGCCTTGGTCAGAACTGGCTCCGGAATGGGACAGGGCTGCCTGTGGC |
| | TCCAGCTGGGGGAGGCTGAGGTGAATAAAGACAGTGATG |
| SEQ ID NO: 753 | ACCTCAGGGCAAGGAATGTTTTCTGTCGCAAGATCCTAAATAACAGACATTCAAAGGAAA |
| | GGTATGAGTTGGAACATGTTAAACTAAAAGTTGCTGCACA |
| SEQ ID NO: 754 | AAAATAATAATAATAAAAATTTGTTTTAGAAAGTGCCCAAACCTTCATCCTTTCCTATCAA |
| | GAAGGAGAGGACTTCCCTGCCAGAGTTCCAAGGCAGGGT |
| SEQ ID NO: 755 | CTGAATAAAAAATAAAGTTTAAAAGGGTTAGTGGATGTAAAATTAATATATAAAGGTCAG |
| | TCATATTTCCATATCTCAGCAGCAAATAACTGGAAAACAT |
| SEQ ID NO: 756 | GAAAAAAAAAAAAAAAAGATCTTACAAGTATAATCATAATATTCTTCAAAATTATAAATA |
| | CTTACACATATAAATTATGAACATGTATGCATATATGAGA |
| SEQ ID NO: 757 | CACTGAATTTTATAGTTAAAAAGAAGAAAAAACTTAGAGACACAGAATCAAAGACCTAAG |
| | AATACCAGAGCACACCAGCAGGATAAATACCAAAAACAAA |
| SEQ ID NO: 758 | ATATATTGACAATGTTACTTCTTTGTTTAGGTTAAGGCATTAAAAATTTATTCATAAAATAA |
| | ATCAATCTATTCATAAATAGAAATAGTCTTTTTAGAAG |
| SEQ ID NO: 759 | ATGAAAACCTGTTTTCATAGACTTATCAGTTCAAACAGCAGTAATTCGTAAATAAACTAGT |
| | ACTTTGTGGTTAAACCAGTAGAGGGTGCACAAGACGCGT |
| SEQ ID NO: 760 | TACCCCACCTCGCCTCCCACCACCCCGTCCAAAAAAAAAAAGATTAAAGAGAACAGAATG |
| | GACTAGGGTCCAAGCTGGTGACTGGGAGTTTATGTAATGC |
| SEQ ID NO: 761 | CCTACCCCTTTGGACTTACCAAACATCTCTGTAATCCTTTAATGTACAAGTAATAGACATCT |
| | ACATCACTCCAAACAACACTCTACCTGCTCCAGGGGGA |
| SEQ ID NO: 762 | TAAAACACAACAAACAAACAATCAAACAAGAAAAAACCACAGAGGCTGTTGAACCTTTTA |
| | GTCAGAACATATAATCTCCAATGTATCATAGTTTCCACAA |
| SEQ ID NO: 763 | TTTTTTTTTTTAAGTTTACACGCTGTTTAATTCAACCACTGTTCTGGGTTTTATTTGTTTGTTT |
| | TTCAAGAAGGAAGTATAAAATCTTTATGTTATAAAT |
| SEQ ID NO: 764 | TGAAAAATAAAAAATAAAGAATATATATATATATATATACACACATACACATATATTTGA |
| | AGATCAAAAGAAATATGTAAATAATTCTTTTCAGCATATA |
| SEQ ID NO: 765 | GGCAAAAAAAGAAAACGAAACACTACCATAAAACCTTGGTAACTTTTAAAATAAATCAAC |
| | ATTCTATCTCCCCAGCTCAAGCTAAAAGGCTTTATTTCTT |
| SEQ ID NO: 766 | AAAAAAAAGAAGGAAAAAAATGCTTTTGGCTGAGTGTGGTGGCTCACGCCTGTAATCCCA |
| | GCACTTTGGGAGGCCGAGGCAAGCGGATCGCTTGAGCCCA |
| SEQ ID NO: 767 | CTCACACACACACACACATACTCAAATGGGGCCTTGAGCACAGGAAAGGAGAGGCAACTA |
| | TAACTGAGCCCACAAATGAAGCCTCTTAGAGCCTCCTGTC |
| SEQ ID NO: 768 | GGGTCAGGGGGGAAGAATTCCCTCTGTGATAGCCCCATTTAGAATAGTGTTTCTGCCTATC |
| | TATGTATATTGCTGTTTTTCCTCCTTTAGTAAAGATGGA |
| SEQ ID NO: 769 | GGTAGTCTGAGTTTGGGCTTGGCCCAGGGTGGGGAAAAGCCCTTGTCCTGGGGCAGTTAAT |
| | GTGCAGGTTTCATGATGGAGCCTGGAGGGTGTTGACTGG |
| SEQ ID NO: 770 | CCCCCAAATGTTTCTATATTAAAAAAATAATGAGAGGCAATTTGTTTTTTGTAGTTACTTAG |
| | CTAGAAAAGGAAAAATATTTTAACCTGTATTTTTTTTT |
| SEQ ID NO: 771 | CTATTTAAAAAAAAAAAAAAAAAAAAGGAAGAAGAAAGGAAAAAAAAACGGTTAAGGCC |
| | AGGAGGAAGCATCTCAGTGGCAGGTCTTAGCCTCATGCCCA |
| SEQ ID NO: 772 | ATAAATATGACACATTACCAACAGAATGAAGGAGAAATACCATATGATAACCTCAATAGA |
| | TGGCCAAAAATTTTTTTGATAAATTTAACATCTCTTCATG |
| SEQ ID NO: 773 | TGTAACAACAACAACGAAAAAAATATATATATATATTTATCAGCCCTAGGAAATCATGTGT |
| | TTTACTACATTAGCAGTAACTCTCCATCTTTTCCTCCCT |
| SEQ ID NO: 774 | CCCAAAAAAGAACGGAAAGGAAAAAAGAAAGGAATGAAAAGGAAGGGAGGAAGGGAAA |
| | GAAAAAGAAAGAGAGGGAGGAGGGCGGGAGAGAGGAAAGGAA |
| SEQ ID NO: 775 | CTAAATTAAAAAAAATACAATAGATAGCATTCAATATAATAACTGCTACTGTTATTATAAT |
| | TGTCACAACTGCTGTTACCTCAACTAATTCATAAAATAG |
| SEQ ID NO: 776 | CTGAATTATTAGGTTGGTGCAAAAGTAATTGCAATTATAAAAAAAAAAAAGAACCTTTCCT |
| | GCTTTTTAAGCAGACAACAATGCATTCCCCTCTGCTGCC |
| SEQ ID NO: 777 | AAAAAAAAAAAAAGAAAAAAAGAAAAAAATAGGAAGAGATATAATCCCACCATACATAC |
| | ATTTAGGATGACCCAGGATTTCCCAGATTAACAAGGTCGAT |
| SEQ ID NO: 778 | CTAAAAAATAAAAATTAAAAAATAAATAAATAAAAATTCCACTGAACTAAAAACAAACAA |
| | AAAAATCCAGAAGGCAAGAATTAGCACTTTGAGAATTGCA |
| SEQ ID NO: 779 | CCACCGCTCCCCTCAAACTCCCATGTTAAAAAAAACTTTTTTTTTTTTTTATGAGTGAATAG |
| | GTAGTTGGATTTTTTTTTTTTTTTTAAGAGACGGGGTG |
| SEQ ID NO: 780 | TAGAAGGGAAGAACACTGCAGAGTAGCTTGCACACCTAGTATCTGTGATATGCTGGTTTTC |
| | CTATTCCTTGAACTACCTTGAAGTTCCTGGTCTGCGGAC |
| SEQ ID NO: 781 | GGGTTTTAATTTTTAATCTATGCATGTTTTTGCTAAACTTTGAGAAATAATTTCATACTACTT |
| | TGGTCATCAAAGACCAAACTTGAATTTTTCCCTTTGT |
| SEQ ID NO: 782 | CCCCACCCCCCACAAAAAGTGAATAAAAAGAAAGGAAAAAGAAAACAAATAAATAAAAT |
| | ATATGTATACCCACAGCAAGGTCTGAGGACAGTATTCAAAC |
| SEQ ID NO: 783 | TTTTATTTTTTCAATATTAAAATGAAATTTAAAACATAAAAAATAAAAATATTGATTATTAT |
| | AAATGGGATATGAGACCTTTAAAACATCACTTTTGTGC |
| SEQ ID NO: 784 | CCCAAAAAATATTAAAAAATAAAAAATAAAAACAATATTGAGGATTTTCTAAAATTTTTTT |
| | GCTACCTCTAACAATGTTGCAATGTAAATCCTTGTATAT |
| SEQ ID NO: 785 | TTGGTACCTGTGGCTTAGTCACCAATAGAAATCAAGATAGTTTCATATAACATTATAGTTA |
| | CCCCTGATATCTCAAAATATTTGCCCTGATCACTATTGC |
| SEQ ID NO: 786 | CCTCCCACACACATAAATTCTCACCAATCTTGAGGGAATGTTAAAATAAGATGTGCTTTGA |
| | TTGGCTGATATTAAGGCATATTAGAAATGAAAGAACTGT |
| SEQ ID NO: 787 | CTGAACTTAACATTAAAAACAAATTAAAAAATGAAAAGATAGGGATGTAAAAGTATCCAC |
| | ATGAGCTGATACCAAGGAGTTGGAGCCAGGCTCCAGGGAA |
| SEQ ID NO: 788 | ACTATTGTTATAATGAACACATTACCAGAGCTCATATTTTGTTTATTCTCAACCTTTTTTCTT |
| | TCATTACAGTGTATGAACTAAGCAATGGACAAAACTC |
| SEQ ID NO: 789 | TACAAATAAAATAAATAAATAAATATAAAATAGAAATTTGGCCAGGTGCAGTGCCTCATC |
| | CTGTAATTCCAGCACTTTGGGAGCCAAGACAGGAGGATCA |
| SEQ ID NO: 790 | AGATAGAAGTTTACCTATGTAACCTGCACTTGTACCCTTGAACTTAAAAGTTACAAAGAAA |
| | AAGTATTACTAGCTTCTCTGTTAGGTATAGAGAGGGACT |
| SEQ ID NO: 791 | CAGAAAAAATTCCAAAAAGAAAAAACCTACTAGCAGTTAAGGAGGAAAAAAATCAGCAA |
| | TTCTACCAGATAAGTAAGGTTAACATTTATTTGATCTTCAG |
| SEQ ID NO: 792 | ATGAAAACCTGCTAAAAATAAGAAAAGTAATCAATTTTGTATCTTTTAAAAAATGTAATGA |
| | TAATAATAATAAAAGTCAGAAAAAAGAAAAGGGAAAACT |
| SEQ ID NO: 793 | TCTATATATTATTTTATATATAAAAGAATAATTAAGATTATTAACATAATAATTTTTAAAAA |
| | GTATTTAGGTTCCAGTTTTATCTATTCTGAATAATGCT |
| SEQ ID NO: 794 | GTAAATAAAAGGAAAAAATGAGCCAACAACCTGAACAGACACCTTACCAAAGAAGATAT |
| | ACAGATGGCAACTGAGCATATGAAAAGATGCTCCACATATA |
| SEQ ID NO: 795 | GAAAAAAAAAAAAGAAGAACAACACTAGAAATGGTAAATAAGAGGGTAAAAATGAAATA |
| | CACTTTTTCTTATTTTGAGTCTCTTTAAAAAATCATTGGCT |
| SEQ ID NO: 796 | ATAGAATATAGCTATATCACTGACATGGATGCACAAGCTACTGTTTTAAAATAAGTTTATC |
| | AATGCTAGTGTAGCAATTTGGTCACGTAAATGCATTTTT |
| SEQ ID NO: 797 | TCTTCACAGCCTAAGAACAGGCTTCCTCTGTGTAAAGAAATAAAAGAAAACACAAGCATG |
| | GGCAGAAGTCAGTAGAACAATTAGGAGGCTCTTACAGTAG |
| SEQ ID NO: 798 | CAGGTAAATCTATTACAACATTTAGGGCCAGGTGCGGTGGCTCATGCCTGTGATCCCAGCA |
| | CTTTGGGAGGCCAAGGTGGGTGGATCACGAGTTCAGGGG |
| SEQ ID NO: 799 | ACCCATTAACTCGTCATTTACATTAGGTATATCTCCTAATGCTATCCCTCCCCCCTCCCCCC |
| | ACCCCATGACAGGCCCCGATGTGTGATGTTCCCCACCC |
| SEQ ID NO: 800 | TCGGGGGAATAAATTTTTAAAAAAAGCAAAATGAGTTTCTTCAGAATATCACATATACCTG |
| | TTTTTCAAATGTAAATACATCTTAAAATGAATTATCAGG |
| SEQ ID NO: 801 | CTTAACTATCTCAGGTTTAAAAAAAGCTGACAATTTTTTTTTAAGTCTCCAGATTCTGCAGC |
| | TAAGTAAGTACTTGAAGGGAAAGGGTGTCAGTCTCCTC |
| SEQ ID NO: 802 | AAAATAGAATATAGCTATATCACTGACATGGATGCACAAGCTACTGTTTTAAAATAAGTTT |
| | ATCAATGCTAGTGTAGCAATTTGGTCATGTAAATGCATT |
| SEQ ID NO: 803 | CAGTCACTTTCAATTGCCAACCTTGTTTTAAAAAAGAAGCATCAGCACTCCCTGAAATCCC |
| | TCTCCGTTTTTCTTACTCTATTCTTATGCTATGCATCCC |
| SEQ ID NO: 804 | TAGTTAAATAAATAAGAAAATAAACAGATCGAGCTGGACCACTATTTTGGCCCCTTTTCCT |
| | TCTCATGTGTCTGGAATACAAATGCCTAGAGGTAAAACT |
| SEQ ID NO: 805 | ACTAAAAAAATGGTTTATAACAACAAATGCCAGTTAATATATAAAGCATAAAGTAATGTTT |
| | GTATGCCTTGAAATGAGTATAATTGATTAAACAAGTATA |
| SEQ ID NO: 806 | TACTCCTACACCATGCAAAAGTTAAAAAAAGAAAACACAAAAAACATACAAAACGTGTTA |
| | ATTGATTACTTATCAGTGAGGCTTCTAGACAGTAGGCTAT |
| SEQ ID NO: 807 | ATGTTTTTGATATAAAAAAAGAAACTTCATGTCTTCAAATCTTTGGGGATTCCTAAAATAT |
| | CTATTTACTAATTGCATGCTATTATTTTTATTCAAAACC |
| SEQ ID NO: 808 | ACTAAACTTTGTTGGTAAAAAAAATAAAAATTAATGATAAATAAAAAATAAACTGCTGAC |
| | TTAAGTTGATTTATAATTCTGTATCTCATAAAATTGGAAT |
| SEQ ID NO: 809 | AATGGTCTGACTCTGTCACCCAGGCTGGAGTGCAGTGGTATAGTCACAGCTCACCACAACC |
| | TCAACCTCCCAGGCTCAGGTGATCCTCCCACTTTAGCCT |
| SEQ ID NO: 810 | AGACTGTGATATCAATCTCTGATATTATGATATCACAGTTATCATAATTTATATGTCTGATA |
| | TAAATTTTTAAAAGATTAAAAAAACCATTGCTTTCGGC |
| SEQ ID NO: 811 | ATGAAAACCCACTTTTCCATGTCAAAAAAAAAGGTAAAAAAAAAAGGCAGCCGGGCATGG |
| | TGGCTCATCCTGTAATCCCAGCACTTTGGGAGGCCGAGGC |
| SEQ ID NO: 812 | CACGGACTAATAAAAAAAAATTGTTAAAAGTAACCCCATATTTTCAATCTTTTAAAAATGT |
| | CCATTATTGATCCTATATATCTCCATAGCTTCCATCTTT |
| SEQ ID NO: 813 | AGTATAAAGAAGAAGAAGAAGGGGGCGAGGGGGAAACAATGGCTGGGTGTGGTGGGTCA |
| | CACCTGTAATCCCAGCAGTTTGGGAGGCCGAGGCGGGTGGA |
| SEQ ID NO: 814 | AAGGAAAGCCTCTTTTCCACAAAAAAGGGGGTAAAAAAACAAGAATAACATCAGCTACCT |
| | TTGTTGCGTTAATTTTGTAGATTAAGTGAAATAAACATGG |
| SEQ ID NO: 815 | AAATATATATATATATGCAGTATTAGAGCAAAGGACCAATAAGAGATAAAAACTAACTGA |
| | ACTACCTCTTAGTGCCTGGAATTTACCTTTTCCTGACTTA |
| SEQ ID NO: 816 | TGATTTAAAAAATTTAAAATTTTTAAATATAAAAATAAAAATAAAATATTTAGGATATTAA |
| | TACGATACAAGGTAGACTTCAAGCCAAAACCATTAGCTA |
| SEQ ID NO: 817 | AAAAAAAAAAAAGCAAGAAAAGAAACAGGCTTTTGCTGAGGATCCACTCCTGCTTCCCCT |
| | GTTGGGCCATTCCTGTTGTGTTGTGTTTGATGTTAGAAAC |
| SEQ ID NO: 818 | TTGCCCCTCAAAAAGAGTATGTATGGTGGGCCCTCTGTATCCACAGATTCTGCATCTGCAG |
| | ATTCAACCAACCTCATTGAAAACATTCAGGAAAAAAACA |
| SEQ ID NO: 819 | TGGGCAATATAGCGAGACCTCGTCTCTACAAAAAATACAAAAAAATTAGCCAGGCGTGGT |
| | GGCGCCTACCTGTATTCCACAGTGTATATTTGCCACATTT |
| SEQ ID NO: 820 | TGATGCTATGTGTTTTTAAAAAATTTTTTGTTTATTTATTTTTTCAGACGGAGCTTCACTCTT |
| | GTTGCCCAAGCTGGAGTACAGTAGCACGATCTCAGCT |
| SEQ ID NO: 821 | TGGCAAAAAAAAAGTCTTAAAAAATGAAAAGGAAGGGTATATGGGAGAAAATTAGGCAG |
| | TACAGGTGAGTAGGGAGAAGATCCTGCAAGGCCTTACAGGT |
| SEQ ID NO: 822 | TTTGACCCATTACCCATCTAAGTTAGATGCTTTTTTAAATGTTTTTTAATTTTTAAATTTTTA |
| | ATTTTTTTCATTATTTATTTTTTATTTTTGAGACGGA |
| SEQ ID NO: 823 | GAAAAAAAAAAGAAGAAAAGAAAAAAAAAAAGAAAAGACAAGACAAGACCAAGTTCTG |
| | AGAGTGCTTGAAGAAGCGGTACTAGGAGAAGACTGCACTGCC |
| SEQ ID NO: 824 | CTCCACAGAAAATTTTAAATCATGTCAATTTCTTTTTTTTTTTTTTTTTTAATCCTCTTGGGA |
| | TTTCAGTTTACATTAAGTAAAATCTATAGATCAATTT |
| SEQ ID NO: 825 | ACCTGGTTCATCTCATTGGGACTGGTTGGATAGTGGGTGTAGCCCATGGAGGGTGAGCCAA |
| | AGCAGGGTGGGGCATCGCCTACCTGGGAAGTGCAAGGGG |
| SEQ ID NO: 826 | AAAAAAATGCTTTGCATCCCACAAGAACTAGATATAGACAAGTGCCATTTTTGCTACACAG |
| | AATGCTTTAAAAAAAAAAATGGTGGGAAATAGAGAAGGG |
| SEQ ID NO: 827 | GAAAAATCTGGCCATTTAGAGTGTCTAAAGCAATGGAAGACCTTGGCGTTACTGCTTGATT |
| | GCCTTGATTTATAGAACCTGCCTTAATTTGACTAAGAAA |
| SEQ ID NO: 828 | CCAAAAAGAAAATACAAATCTTACCTGAAGATCTTCTGAGAATATGATAAGTAGGAAAGT |
| | ACTTAACCTAGAGTAGGAAAGTATTAATAAAAGAATTAGG |
| SEQ ID NO: 829 | TAAAAAGAAAAATAGTAGGCTAATAAGGGAAGGCTATTCAGATGTTGTATTAGTTTTGAA |
| | CTATATCTTGTAGAATGTGATCAACCAAGGCAAAGCTGAC |
| SEQ ID NO: 830 | ACCCCCGCACCTCCGCAAAAAAGCTACCACTTGTAGCTGGGTGCAGTGGCTCACGCCTGTA |
| | ATCCCAACACTTTGGGGAGGCCAAGGCACAAGGATCACT |
| SEQ ID NO: 831 | CCTACCCCAAAAAAAGAAAGAAAAACCATAAGCCTGTGTGTGTGCTCCCTGGACCCTTCTT |
| | TTCCGCAAAACCTTGCTGAAATCAGAGACAAGGACCTTC |
| SEQ ID NO: 832 | GCAAAAAAAAAAAAAAAAATGCATACATATAATTTATAAGTCCAACATGCTCTGTTTTCTT |
| | AAGTCAAATAAAATAGAAGCCATAGTTGTTATGAGTAGA |
| SEQ ID NO: 833 | GTAATTGGGTAACAATTTTCCATAAATTGAAATGAGAAATGTTAGAAACACACAACCAGTT |
| | AACAGTAAGTGGCACAACAAAAACAGCAAAAGCCCACAT |
| SEQ ID NO: 834 | GAAGAGGCATCTGTGCCCAGCTCGTGGCCTGTTCTTTGCACTTGCGGTGGAGATCCTCTTCT |
| | CCCACAGCTGTCCCCCCGGGGCCTGCTCCCAGGTGGGG |
| SEQ ID NO: 835 | CTGAATAAAAATAAATAAATAAAAAATAAATGTAAAAAAAAAAAAGACCATCATAGTACT |
| | GTGAAGAGCCTGGTCATCATCCTTTTTTATGTAAGGAGTA |
| SEQ ID NO: 836 | TCCCCAACAAAACAAGAGAGAAATAGTCCTCATTTACTATTTAATAACCCAGTGTCCTTAA |
| | ACAATCTTAAACATGCTGAGAAGTTTAAAGAATAGTACA |
| SEQ ID NO: 837 | CCCACAACCCACCAAAAAAGTATTGAGGATATAGATACAACCAATTTCAAGCTCAAACTG |
| | AGATCAATAGTGTCATAACAATTCTACTGGTTTACATAAA |
| SEQ ID NO: 838 | AAAAAGTAATTTTTTTTCTTAATATACAAAATAAAAATAAATAATTTCTACCCTGTTTAAAA |
| | ATCTCACCTCCAGATCCTCCCCAAAGTTAGCAGGACAG |
| SEQ ID NO: 839 | ACTAAAAAAGGGTTTAGAGAACTAAAGGAAACATATGATAGGCACATAACAAGCCAGGA |
| | TGCAACTTCTGCTATAAGACATAAAGCACAATTATCTATTA |
| SEQ ID NO: 840 | CTAATGAAGGACAAGAAAAAGGAGACTGTCCTGGAGAGATGAGGACTGAGAAACCCAAC |
| | CCAGCCCCTGGCTCCAAGGAGTCCCGGCCCAGCCCTGGAGA |
| SEQ ID NO: 841 | CAAACCACTCTCTGAGTGACTCTCTTCATCTGATTAAATGAAGATATTAGCAGTTCATAGG |
| | ACTGTTGTGACAAAGCAGGTCACCAACAAATGGGAGCTG |
| SEQ ID NO: 842 | AAAAACGGAAACAAAAAAATAGGAATTTGATGGGGTGGGGTTGGAGATGAGCAGTGTTCT |
| | GAAGGCATTTTTGAGTAATTATTACATATACTGTACACTG |
| SEQ ID NO: 843 | CCACTCCCCTCCCCCGCCCAACCAAAAAATAAATAAATAAAGTAGGTAGAAAAGAGAAAG |
| | AAGCTTCAGTTATAAAGACTGGATAGAAGGTAAAACCATT |
| SEQ ID NO: 844 | CTGAATTATAAAGAAAAAAAAACTAATAAATTCAATAAAGTTGCAGGATAGAAAAATCAA |
| | CATCCACTAATAAACTATCTAAAAAGGAAATTAGTAAAAC |
| SEQ ID NO: 845 | CATGCAGGTAAATAAAACAAATCAAAACTAGCAAACAAAACTAATTAAGAATACTAGGAG |
| | GGAAGGAATGATTGGACTAATGAACTGAGAAATGGAAAAT |
| SEQ ID NO: 846 | TAGTAAGAAAGAAAAAAAAAAAAGTACATGGGGTTCATACTACTTTGTAGTTTTCGTTAA |
| | ATGAAACACTCTCACACAAAAGGTATCTCCAGAATTTACA |
| SEQ ID NO: 847 | GGTCTTCGTGGTTTAAGAACAGGTTTTCTCTGCTTATTTTTATTAAAAAAAAAAAAATTATG |
| | TGAGTCAGTGGTTCTCAAGCAGCAGTGATTGTGCCCCC |
| SEQ ID NO: 848 | TGCTAAAAAAAAAAAAAATAATCAATTGGGTATAATAAATCAGTCATCCATTTCTCATCCC |
| | CACAATCATTCCTGTCTGACAAAAAAGTTTAGGAGAGTT |
| SEQ ID NO: 849 | AAAAAAAATGAAAGAAAGAAAGGAAGATAGGAAGGAAGGAAGGAAAGAGAGAGAGAAA |
| | GAAAGAAGAAAAGAAGAAAAGAAAGAAAGAAAGAGAGAAAGA |
| SEQ ID NO: 850 | ATTATTAAAAGAAAAAAATTAAACAGCATTGCCGTATGATCTAGTAATTTCTATTCAGGGT |
| | ATACACCGCAAAGATTTGAAAACAGATGACTCAAACAGA |
| SEQ ID NO: 851 | TAAAAATTTTTAAAAATAAATAAATACATAAATAAATAAATGTGTGTATATGAATCCATGT |
| | GCATGCACGTATGTGTAAATATTTCTCCAGGTATCCATC |
| SEQ ID NO: 852 | CACCATGAAAAATAAATAAATAAAATAAATTCAAGCTACAAATGAACGTAAATCCTAATA |
| | CGATTGCCTTATGGCCAAGAGATTTACTCCTAGTTCTTCT |
| SEQ ID NO: 853 | GGAGGCCTTGAATTTGATTTACTTTTCCCACTTCAATCCCAAATCTAACCCCATTTACGTCT |
| | CTTGAACTAGTTAGCTCTGGGGGCAGCAGGCAGTGGCC |
| SEQ ID NO: 854 | ATGAAAAACTGAAGGAAAAAAACAATGCAACATGTGATTCTAAAGTGGATCCTCCTATTA |
| | TAAAGAATATTCTCTGGGCATGGTGGCTCACATCTGTAAT |
| SEQ ID NO: 855 | ATGAAAACCTGCTCTACCCTCCCCCCAAAAAATATGTCTTGATTGCTTTTGCTGATGTTATG |
| | TTGGAAACATATCCTATGGCAGATGTGATCTGATGACG |
| SEQ ID NO: 856 | CTAGGAGTTCGAGACCAGCCTAGGCAACATGGCAAGACCCTGTCTCTACAAAAAATATTT |
| | AAAAAATTTAGCTGGGCATGGTGGTGCTTGCCTGTAGTCC |
| SEQ ID NO: 857 | ACATTAAGGCAATCTATTGATGGTAAACTTTTATCAGATGCCAATGTAGTCTCCCAAGATT |
| | TCCTGGAGGGGAAATGGATAACAACACTGAACTTACTGA |
| SEQ ID NO: 858 | TGATGAAGTCAAATGAACAATTCCTTACATTTTGTTTGTGCTTTTGGTAACTTGCTTAAGAG |
| | TTCTTTCCCATACCAGTGTCATAACCTATAGTTTCTTC |
| SEQ ID NO: 859 | TTCACTAGATTTTAGAATAGTAGACTTAAGTCTTTTACCAACTGCCTGACCTTGAGAAAAA |
| | AATATGAACCTCTCTGAATCTTACTTTCCTCCCCTTTGT |
| SEQ ID NO: 860 | AAAAATAAAGTAAAATTATGATGGAGGTCATGGTACCTCCCAGAGTTGTGCAGCTCATGTT |
| | TTGCACAACCATTTAGGGGTTCTAATGGAGCTCTGGGAT |
| SEQ ID NO: 861 | ATCCTGGGTTCTTCTCCAGGCTTCGTGACATTCAGTCATTCATTCATTTCTTCACTAAGCAT |
| | TGCACAGTTCCCCTGAACCAGGCACACCAGGCACTGGC |
| SEQ ID NO: 862 | CAAAAAAGTTTAAAAAAGAAAAAAAATAGGGGACTGATTGAATAAACTATGGCGCAGTA |
| | AGATGCAGAGATAAAAATGCGAATTTTGTCAAGAACCAAGA |
| SEQ ID NO: 863 | CCTCTGCCTCCCGGGTTCAAACGATTCTCCTGCCTCAGCCTCCTGACTAGCTGGGACTACA |
| | GACGCGTGCCACCACACCTGGCTAATTTTTGTATTTTTA |
| SEQ ID NO: 864 | AAGAAAAAAAGAAAAAACCAAAAGCAGTGCATTAGGGGCAATTCCTCCCCCTGCCTCCTC |
| | TCTCCAGTGCTGATGGGCTGAGTGTGGGGAAAGGCGGCTC |
| SEQ ID NO: 865 | AAATAAATAAATAAATATAAATCTTTTGCATCAAAGGACATTATCAAGAATGTGAAAGAC |
| | AACCTACAGAATGGGAGAAAATATTAACAAATCATATATC |
| SEQ ID NO: 866 | CACCCCCCGAAAAAAGAAAGTTGGTAGATTGGTAGTATACAGGATATATCAGTATCCTTTG |
| | GGTGTTTAGGTATAAGGAGATCTGCTTAGAAATTATAAC |
| SEQ ID NO: 867 | ATATCTGAGATAGGCCTCAGTTAATTTAGAAAGTTTATTTTGCCAAAGTTGAGGACACGCG |
| | CCCATGACAGCCTCAGGAGGTGCTGATGACATGCGCCAA |
| SEQ ID NO: 868 | TATCACTGTTGGTCAAATGATAGAAGTCATACTTGGGATGTGTGGCTATTGTTTTGCTTTGT |
| | GACTTACAGTTCTCTAAAGTAGATATGACATTATGGGG |
| SEQ ID NO: 869 | GTTAAATTAAAAAATTTTTAAGTTATTTTAAAAATATAAAAAACAAGATTTGACTGTATTC |
| | TGCCTTGCGCCACTCTTGTTTGTCTTTCACTAGTGCAAA |
| SEQ ID NO: 870 | GGGTGCAAGAGAGAGTTCCACTCCCAGAGAGAGGGGTGAGGGGATGAGATGGTGTGAGT |
| | AGCAGACTTGGGTGCCAGTGAGCAAACAAGGAAGAGGAGGC |
| SEQ ID NO: 871 | CGAATTCCAAAAGCCCCATCTTCATAGTACCCGGGCTCTAAAACAAACCAAATCCCAAAA |
| | ATGAGAAGCAGCCCACGATGAAGATGTCTACAGAGAGAAA |
| SEQ ID NO: 872 | TCCTGATACCCCTCAAAAAGGGGCACATTTTGACACAGAGACAGACACGCATACTGGGAA |
| | AATGCTGTATGAAGCTGAAGGCAGAGATTGGGGTGATGCT |
| SEQ ID NO: 873 | TTTTTTTTTTGCAGAGGGGGTGGACTTTCTGGTTTTAAAAAAAAAAAAAAAAAAAAAAAAA |
| | CCCCTCCACAAAAATTTTTAACAAAAAGTTTAAATGTAA |
| SEQ ID NO: 874 | TCACAAAAAAAAAAAAAAAAAAAAAAAAAGCAAATCCTTAATATGATTTTCTTCTCACAA |
| | AGGAAGCAAATCCTGCTCTGCTCACTTCCATAAATATTAA |
| SEQ ID NO: 875 | AAGGGAGATAAAGTTTAAAAATGAAAAAAATAGGGAATAAAAAGCTCTAGCCCTACCATC |
| | CAATTGGCTGGGGGAATTTAAATAAATAAATAAATAAATA |
| SEQ ID NO: 876 | TGTCAACTTTCTTTTTTTTCTTTTCTTTACTTTTCTATTTTTTTTTTGAGATGGAGACTTGTCC |
| | TGTCGCCCAGGGTGGAATGCAGTGATGCGATCTCGG |
| SEQ ID NO: 877 | TTAAAAATAAAAACAAAAACCTAAGCCATAAGTCCTGGAGAAGAGGTACAGAGATGCTGT |
| | ATCACTCAAGAGGAATGCCTGGGTTTAGAAAATGGGCATC |
| SEQ ID NO: 878 | CCCACCAAAAAAAAAGTCTACCTTTGGTGAAATGACATCATGTCTGGGATTTGCTTTAACA |
| | TATTTCAGCGAAGCAACCTATGAAATGGGAGAAAAATAT |
| SEQ ID NO: 879 | ATCTTTGAAGTATCTCCACAATCATCTGTGGGGTACATAATCTACAGCCGGAGATACTTAA |
| | GGCTTTTTATTTTGTGCCTGGTATAAGATGGCAGATGTG |
| SEQ ID NO: 880 | TTGCACATCTGTGTTCTAGATGTACAATTCTAGATATTTTCAGTTTTACTCAACACAAATTT |
| | CTTCACTCTTGTCATCTGCTAACTTTATTCATAGTGTG |
| SEQ ID NO: 881 | TCTTAGTCTTCATTTGGAATTTATAAAAAATTTTAAAAATCACCACATTTTCCAAAAGTTTA |
| | TGCCAGAAAATCATTCACTATTTAAAAATGAAGTGCAA |
| SEQ ID NO: 882 | ATGAAAACCTGATTTTCCACAATAAAGAAAATAAAACAATAAATAAAATTTTAAACAAAG |
| | ATTTATAGAGCTAAAACAATAAAATGCCTAAAGTAAAGCA |
| SEQ ID NO: 883 | ATGAAAACCCCATTTTCCATGACAATAAAAAAAAGAAAACAATAAATAAAATTTAAAAAT |
| | AATTTATCTAAATGTATAAAATGCCTAAAGTAAAACAAGA |
| SEQ ID NO: 884 | CTGAAGAAAAAATGATAATAATCAAGGTAAAATCGCAGTGCTTGATTTATGGCATGACCTT |
| | TGACAGAAGCAGTAAAACTTTTGTATGGAAGAAAATCAG |
| SEQ ID NO: 885 | AAGAAAACCAACCAAACAAACAAAAAAAAGCTGTATTGTATACTTAATAATTTGCTAAAA |
| | GAGTAGATCTTATGTTAAGTGTTCTTATAATAAACAAACA |
| SEQ ID NO: 886 | TTATGCCACTACTGCCTTCTAGCCTTTGTGGTTTCTGATGAGAAATCAGCTGTTAATCTTAC |
| | TGAGGATCTCTTTTATGTGAGACGTTGCTGCTCTCTTG |
| SEQ ID NO: 887 | ATGAAAAACTGCTTTTCCACAGGGGGAAAATACTGTTTACTGCCCATCTTTGAAAAACATT |
| | TTACTTAGAGCATAAACTTGAAGCTAGATCCTTTTAACC |
| SEQ ID NO: 888 | CTTATGATGTTTGTTGCCAATGATAGATTGTTTTCACTGTGCAAAAATTATGGGTAGTTTTG |
| | GTGGTCTTGATGCAGTTGTAAGCTTGGGGTATGAAGGT |
| SEQ ID NO: 889 | GCCCCCAAATATAAAAAAATAATAAGGCATTCACCCCTGAACTTTAGCAGGGCTTCCAATC |
| | GCCTTTAAGCACTCATTTAAAAGAAAAGTCTTTTATTCC |
| SEQ ID NO: 890 | GTATATTGTTAAATAATTTTTTATAATTAAAATGAAAGAAACTATCAACAGAGTGAACAGA |
| | CAACCTACAGAATGGGAGAAAATATTTGCAAACTATGCT |
| SEQ ID NO: 891 | CAAAGGGAAAAAAATAAGTTGTATTATTATTATTATTATTTTGAGACAGAGTTTCGCGCTT |
| | GTTGCCTAGGCTGGAGTGCAATACCACGATCTCAGCTCA |
| SEQ ID NO: 892 | TTAGAAAAAATATGTATAGACAGCACATCAATCATGATTCCAAGTTGATAGTTGCTTAGAA |
| | AGATTTTTATACATTAGGTACTCTTAGTACAAATATATC |
| SEQ ID NO: 893 | ATTTTATTTTATTATTATTATTTTTTGAGATGGAATCTCACTTTGTCACCCAGGCTGGAGTGC |
| | AGTGTTGCGATCTTGGCTCACTTGCAACCCCCACTGC |
| SEQ ID NO: 894 | CCACCCGCCAAAAAAAACTATATTAGGAAAATTTATAATGGAAGCAAAAATGGATTAGAT |
| | AGGGTATTTTTGTTGATTTCTACAGAAGAATAAACTGATT |
| SEQ ID NO: 895 | TCCCCTCAAAAATAAATTTTTTTTTTTTTTTGAGACAGAGTTGTCGCTCTTGTTGCCCAGGC |
| | TGGAGTGCAGTGGCGCGATCTTGGCTCACTGCAACCTC |
| SEQ ID NO: 896 | GTGATAGGAAAATTTTCGCCAGCATAGTAAGAGCAATTTGGGTTTCCCAAAGTGCAAGGT |
| | GAATATTTCAATGGGTAAAACAAATAATTTTCAATGAAAA |
| SEQ ID NO: 897 | CAGGAAGAAAAGAAGAAATACACTATACACTACCCATGAATTATCCCAAAAATGAAATTA |
| | ACATTTATAATTGCATAAAAATTATTAGGAATAAATTTAA |
| SEQ ID NO: 898 | GAAAGTGAAAATAGAAAATAATATGAATAGTCCTCTATTGGAAGGATGATTAATAAATGA |
| | TACCCCTGTACTCATAGCTCCATCATATTTGACTGTAAGG |
| SEQ ID NO: 899 | CTGAATATTTCAGTATTAAAAAAAACAGAGGAAATGAAAGTGCCCTCTATTGGCAGGTTTA |
| | ACTGCTTTATACTCTCTTCCACTATTCTGTTGGATGAGG |
| SEQ ID NO: 900 | AATAAAAAAAAAACACTTTAAAAAATATTTTTAAAGTTCACTTAAGTTGCTCATTTTATAA |
| | AATGGAGTTTTTCATATTTATGTTGGATTTCATTACTTT |
| SEQ ID NO: 901 | ACACGCTACAAATACCTGAGACTGCGTAATTTATAAGAAAAGAGGTTTAATTAGCTCACG |
| | GTTCTGCAGGCTGTACAAGAAGCACAGCAGCTTCTGTTCA |
| SEQ ID NO: 902 | CCTTACTGGAAGTTGAAAGGTAGCTGTTATTATGATCGGCGCTGGGTCTGGATGTGTGGTG |
| | TTCAAAACACGGGCTGCTGGGCAGTTCGCTTTCGTTTTC |
| SEQ ID NO: 903 | ACACACCAAAAAACAAACAAAAAAAAACAAAAAAACCCACCTTAAACCTACTTTTACACT |
| | ATTCTGTAACTGAAGAATTATCATTTCAGTTGAGCAATCT |
| SEQ ID NO: 904 | CTGGCCACCCTGCAAAAAAGAGAATGGAATATTCTAGTTAATTAAATTTCCTGAAGAATGT |
| | AAACTTTTAATACACTTTAGTCCTTGGTGAAAATCATTT |
| SEQ ID NO: 905 | CCATCTTATTTACTTCCTTTTGATATTTCAAAATTCATGCAGACCAGGCATGGTGGCTCATG |
| | CCTGTAATACCTGCAGTTTGGGAGGCTGAGGCAGGAGG |
| SEQ ID NO: 906 | CCAAAAAATTGTTGAAAAAAAAGAAGTTGTGTGCTTATCCCCAAAATAAATAACTCACTCT |
| | TCTCAGCCTTTTATTATTCTGAGTGAGACTTCATTTGAA |
| SEQ ID NO: 907 | AACCCTCATGAGACCTCAACCATGCTCAGATCTCCAAACTGAGAAAATACATCAGCTACCC |
| | AGTCTATAATATTTTGTTACGGCAGCCCATGCTAAGATA |
| SEQ ID NO: 908 | CTGAATTGTTTTAGAAAAAAGTGGGAAGCAAGGGGAAAGAGATTCAGTAGGTGAACACAT |
| | TTAGATCATGCAGGTGAAAGGACCACACTACTGTGGGTCA |
| SEQ ID NO: 909 | AATAAAAATACAATACAATACCATACAATACAATATATAATACAATACATTCCTCTCCATA |
| | AATTCCTCTTCTGGAAAGCCCCCCTCAAAAAAAAATTCC |
| SEQ ID NO: 910 | CCAAGAAAAAAAAAACACATTAACATGTTCTTTTATCTCACAGAATTGAGCATTTATTCCC |
| | TGTCAGGTAAGAAATATATTAGGTGAAAGTGGCCTTTCA |
| SEQ ID NO: 911 | GTTAAAGAAAAAAAAAATCAAACATTTAGAAAATCTTGTTTCCTCTGTCTCCCCTAACTTT |
| | TGGGGGAGATGGAGTTTTTGTTTTTGTTTTTGTATTTTT |
| SEQ ID NO: 912 | TAAATAAAGAAAGAAAAAGAAAAAAAAACGTCTTCCTCAATCAAATCCGAGGTGGTTCCT |
| | TCCCCCACCAGGCTGGCTGGGTGGGGAGAGAAGGCTTGAA |
| SEQ ID NO: 913 | CTGAAAACAAAAATTTTTTTTATAGAGATGGGGTCTCACTTTGTTGCCCAGGCTGGCCTCA |
| | AACTCCTGAGCTCAAGCAGTCCACCTACCTCGGCTTCCC |
| SEQ ID NO: 914 | GGGGAAAAAAAAAAGGAAAAAAAAACTCATTTGGAGTTTAAAATATAATTAATTAAAAAT |
| | TAATCCTGTGGGGTCACGTATCTAAGGGAGACTTTAACCC |
| SEQ ID NO: 915 | TGGTACATGAGATTGGGCAGGAAGAAATGTGGAGAATAGAGTCACCAGAACCACCCATGT |
| | GGAAGAGCACTAGTGTACGAGAAATTATGGAAGAGCCTTG |
| SEQ ID NO: 916 | ATATGTGGTAATCCAACAATAGAAATTATTTTTAAGTTTGTGTGTTCCTTTTTCTGTTCAAT |
| | GGTGCTTTTGATATTGTTGTAAAGCAGTGACTAGCAGA |
| SEQ ID NO: 917 | ATATAAAGCTGTTAAAAAATCAGATTGACTTCATTTAGGGTGTTTCTTACAGATATCGTTTA |
| | AGTTTTCGGTTCTGCTTGTAAACGCTTCAATCGCTCAT |
| SEQ ID NO: 918 | ATTCTCCTGCCTCAGCCTCCCGAGTAGCTAGGATTACAGGCATGCACCACCACGCCTGGCT |
| | AATTTTTTTTGTATTTTTAGTAGAGACAGGTTTCTCCAT |
| SEQ ID NO: 919 | CACGTGAAAAAAAAAATTATAATTCTACCCAGAGATAATGCACTATTAATATGTGGGCAA |
| | TCATCCCTGTGGTTTTCCTTCTCCTTATACTTGCCCCCAC |
| SEQ ID NO: 920 | ATGCAAGTTTACAAAAACCAAAACTATGTACACAAATTACATGCTAAAAAACCCAAATAC |
| | TAAAAATTATGAACATGACTGAGTGAAACTATGAGTCTTT |
| SEQ ID NO: 921 | CACTTTGGGAGGCAAATCCCAAGGTGGGCAGATCACCTGAGGTTGGGAGTTCGAGACCAG |
| | CCTGGCTAACATGATGAAACCCCGTCTCTACTAAAAATAC |
| SEQ ID NO: 922 | CGAGGTATTAAAAAAATAAAATAAAATAAACAACCAGATCTCAGGTGAACTCAGAGCGAG |
| | AACTCACTCATCACCAAGGGGATGACGCAAAGCCATTCAT |
| SEQ ID NO: 923 | AAAGAAGTTTAACTGACTCACAGTTCCACATGGCTGGGGAAGCCTGAGGAAGCTTACAAT |
| | CATGGGGGAAGGCGGAAGAGAAGCAAGGCACGTCCTACAT |
| SEQ ID NO: 924 | AAAACAATAAAATAAAATAAAATAAAAGTTGCTTAAATAGAATCAGGTGCCTGTCTCCAG |
| | GCTTCTCTGACAGGCGGGAACAGGGAGGCGGGGGGCCCAA |
| SEQ ID NO: 925 | TCCTAATTGATAAAATAAAAATATTTTTGAAAGGGAAGAATTAAAAATCATGGGATTAAAT |
| | GACAGGAGGAGCCAATCTGGGTATTCATAAAATGACTGA |
| SEQ ID NO: 926 | CCCACCCCCACCAAAAAAAAAAAAAAATCAGGCTGGGCACAGTGGCTCATGCCTGTAATC |
| | TCAGCACTTTGGGAGGCCAAGGCAGGCAGATCACGAGGTC |
| SEQ ID NO: 927 | CTGAATAAGAAAAAAGAAAAGAAAAGAAATTAGCTGGGTGCGATGGCTTATGCCTGTAAT |
| | CCCGGCACTTTGGGAGGCTGAGGCAAGCGGATCACTTAAT |
| SEQ ID NO: 928 | GGTAGGCAGCAAATATAGTGTCTGTGAGATTTGAGGCTGCCTTTCTTCTCTGGGATAGACC |
| | ATCTTTTGATTCTTTTCATTGCGATTAGTTGGAAATTTC |
| SEQ ID NO: 929 | CTGAAAAGAAAAAAAAAAAAGAGGAGAACAGCAGACACCAGGGCCTACTTGAGGATGGA |
| | GGGTAGGAGAAGGGAGAGGATTAAAAAAACTACCTCTTCGG |
| SEQ ID NO: 930 | CTGAAATAAAAAAAAAGAAAAACAAAGCTCAAGTTTGCCTCATTTGGCAAATGATTCCCA |
| | GGTGAAAGCTAGCGTTCCATGTTCTGCCTACTACTCTCTA |
| SEQ ID NO: 931 | CACTCCTAAAAAAAAAATGGAGAAACAATTATTATATTTTCAGATGAAAAGACTGGGGGA |
| | ATTTATTACTGGTACACTTGCTTTACAAGAAATGGTAAAG |
| SEQ ID NO: 932 | CCCCTCCAAAAAACGAAATTAAAAAAAAAGTTAAACAAAACAAAACCCAGGCTGGGCGC |
| | AGTGGCTCACGCCTGTAGTCCTAGCACTTTGGGAGGCCGAG |
| SEQ ID NO: 933 | AGCAGGTGGATCCTTTGAGGCCAGGAGTTCAAGACCAGCCTGGGCAACATGGCGAAACCC |
| | ATCTCTACAAAAAATACAAAACTTAGCCAGCACGGTGGCA |
| SEQ ID NO: 934 | CAAAAGGCAAAAAAAAAAAAAAAAAAAAGACCCAAACAGAAGCCTAAGAATCAGGTACA |
| | TCTTTCCAGTTGGTCTCTTCCTCTTGGAAAAGTCCATATCA |
| SEQ ID NO: 935 | AAAAAAAAGAAAATTATTGAGATTATTGGCCAGATGCAGTGGCTCACACCTGTAATCTCA |
| | GCACTTTGGGAGGCCGAGGTGGGCGGATCATGAGGTCAAG |
| SEQ ID NO: 936 | TGGGTTTTTTTTTTTTTTTTCTAATATTAAAAAAAAGAAGAGGCTGGGCGTGGTGGCTCACG |
| | CCTGTAAGCCCAGCACCTTGGGAGGCCAAGGAGGGTGG |
| SEQ ID NO: 937 | CCACCACCACCACCAAATTAAGAAAAGCCGTAAGAATGTGTGATTAGTCCTTTCTGAGTCT |
| | TTAATGCTTTTTACAATAAGGATGTATCGTGTTTTTAAC |
| SEQ ID NO: 938 | CAAAGGAAAAAACAATAAAAAGGCAGTAGGAGCAGGTCATTCCCATTCATTCAAAAAACA |
| | GTTGTCTGGCTAGCACCGTGGCTCTCACCTGTAGTCCCAG |
| SEQ ID NO: 939 | CCGAATTTAAAAAAAGAAAAAAGAGAATTCATTTTTAACCATAATGTGCAAATACTGTATT |
| | TGATGTATCCTGTACTCTGTCACTTGTCTTCGAGTGCCA |
| SEQ ID NO: 940 | AGCTGCCCCATGGGCTAAGCCAGGGAAGGCTTTCTGAACAAGTGAGTGTTGCAAACCTGC |
| | AAAGGTTGGAGAGGAAGGTGGAGGGACACTCCAGAAAAAG |
| SEQ ID NO: 941 | GTTTTTGTTGTTGTTGTTGTTGTTTGTTTTTTGTTTTTTTTGAGACGGAGTCTCACTCTATCAC |
| | CCAGGCTGGAGTTCAGTGGTGCAACCTCGGCTCACT |
| SEQ ID NO: 942 | TAATTTTAAAAAAAGAAGACAAGCACAATTCAGATAAGTAAAATAGAATTAATTAAATGC |
| | ACGAAAGAGAGGAATAACCAAATACTTTGAGAGCTGAGCT |
| SEQ ID NO: 943 | TGCAAATAAAATAAATATATTAATAAAATTTAAAAATGAAAAGCTTTTGCCTTTTTATTAC |
| | CTCCCAAAATACACACACACACACACACACAAACACACA |
| SEQ ID NO: 944 | TGACCTTGTTAAGAGGCAATTTGATGTCTTCCTGGGTGATTTAAGAGCAACTTGATTGTGA |
| | CAATCATGAAAATGGTGTGCAAATGAATGAACTTTTGGT |
| SEQ ID NO: 945 | TGAATTAAAAAAAAAAAAAAAAAAAAGAAGAAGTTACAGGTTCTGCCCACACTCAAGAG |
| | GAAGAGATTATATCAGGGCGTGAATACCAGGAGGCAGGGAT |
| SEQ ID NO: 946 | CTACCACCCCCAAAAAGCCCAAGTCTCAAGAGGTAGTCTGGAAAAAAGTGTTTTTGGATTT |
| | ACCTACATTTTTTTTTCCAAGATGGAGTCTCGCTCTGTC |
| SEQ ID NO: 947 | CTGTGCCAGCTCGTGGCCTGTTCTTTGCACTTGTGGGGAAGGGGTCCCCTTTTCCCACAGCC |
| | GTCCCCCCGGGGCCTGCTCCCAGGTGGGGTCTGGTTTT |
| SEQ ID NO: 948 | CTCACGCTTAAAATAATAATAATAATAAAATAAAATATATTTTAAGTCTCACGCCTGCAAT |
| | CCCAGCACTTTGGGAGGCTGAGGCGGGCAGATCACTTGA |
| SEQ ID NO: 949 | CGCCCAAAAAAATAAAAATACAAAAATCAGTCGGGCGTGGTGGCTCACGCCTGTCATCCC |
| | AGCACTTTGGGAAGCCGAGGTGGGTGGATCACCTGAGGTC |
| SEQ ID NO: 950 | TGAATTCAACTGAGTATCCAGTTTCATAGAAATAATAATAACATCTTCTATAAAATGTATT |
| | CTTGTGTATTAACACATGGGGCTCTCTAGTACTCCTGCT |
| SEQ ID NO: 951 | GTCTAAAATATTAAACGACAACAACAACAAAAAAATCAATGGGGAGAGCTAGGACATGG |
| | AGGCAGGACATTAGAGCCATTTGATTTAAAAAATCATGGTG |
| SEQ ID NO: 952 | GCTTTTTATTTTATTTTGTTCATTTTTTTAATTTAAAAAATAGTGAGAAAAGAAGAAAATAA |
| | GCCCAATGCCAGCAGAATGAAGGAAATAATAAAGATAA |
| SEQ ID NO: 953 | ATGAACCATCTTTGTTTAAAAAAACGAAAACAAACAAAAAAACAAAAAAAAACTTAGCTC |
| | CATGTAAAGGACTTTACATCTAACTTTGTGTTGAATGTAC |
| SEQ ID NO: 954 | AGAAAGAAAAAGAAAAGAAAAAAAAATATTTTAATGGAGAGAGTCACCATGAGATACAA |
| | ATGCACAGATGGGAGGGAGAGGCAGCAGTTCTGGAGGAGAA |
| SEQ ID NO: 955 | ACCTTATTCACGCCTAAAAAGTAGACTGACTGTGGGGTGGTCGTGTTTTTTGTTTCTTGTTG |
| | GTAGGTGGTGAATGCGTTTTTTTCGTTGTTTTCTCCGT |
| SEQ ID NO: 956 | GAAAGTAAAAATAGCCGGGTGCGGTGGCTCACGCCTGTAATCCCAGCACTTTGGGAGGCT |
| | GAGGCAGGCAGATCATGAGGTCAGGAGATAGAGACCATCC |
| SEQ ID NO: 957 | ACAGCAACAATAAAAATAAAGCAATAAACGTAACAACATGAATGAATCACAACGCATTAT |
| | GCTAAGGATAGGAAGCCAGACTCAAAGGGCCATAGAATTT |
| SEQ ID NO: 958 | CCACCAAAAAATATATATATATATTTTTAAAGAAAAATGGTGAGGTAACTGAACTGTTAGC |
| | ATGTGCCAAAGTTAGGGTCTTGAGAACCTAAAGTTTTAA |
| SEQ ID NO: 959 | ACACCTTTACACCTAAAAAGTAGACTGGGGGCCTGGCGCGGTGGCTCACGCCTGTAATCCC |
| | ATCACTTTGGGAGGCTGAGGAGGGCGGATCACGAGGTCA |
| SEQ ID NO: 960 | TGAATATTAAAGAAAAAATACAGAGAAATCTCATACTTTTAATACAATCAGGGTTTCACAT |
| | TGATGAAATCCCCCCTTTACTTTTTTTGAAGTATTGTTT |
| SEQ ID NO: 961 | GTGAATTTTAAAAAGAGGAGAGAGAGAAGCCGCAGAGGATAGTGCACAGAACTGAGTCC |
| | AGATCCCAGTTCTGTCTTTGTTTTTTTGTTTGTTTGTTTGA |
| SEQ ID NO: 962 | GGGGGTTAAAACACAGTGTTACAGGCCAGGCGTGTTGGCTCACCCCTGTAATCCCAGCACT |
| | TTGGGAGGCCGAGGCAGGTGGATCACCTGAGGTCAGGAG |
| SEQ ID NO: 963 | TTCAAAAAACAAAACAAAACAAAACAAATGGCCTTTTATTTCCTAGTGACTGAACTCTTTT |
| | AACATTTGATTAACGTCCATTTCAAAAATGGCTATATTC |
| SEQ ID NO: 964 | AGAAAAAAAAAAAAGAAAGAAATTATAGGCCGGGCACGGTGGCTCACGCCTGTAATCCTA |
| | GCACTTTCGGAGGCCGAGGCAGGCGGATCACGAGGTCAAG |
| SEQ ID NO: 965 | AAAAAGGTAGAGGAGAGAGAGGTGCGTGGAAGAGTGAGCACAAGAAAGGAGGCATCTTA |
| | CAGTTTATAAACATCTGAACACTGGAGCTCTATCAAGATTC |
| SEQ ID NO: 966 | GGGGGTTAAAACACAGTGTTACGGGCTGGGCGTGTTGGCTCACGCCTGTAATCCCAGCACT |
| | TTGGTAGGCCGAGGCAGGTGGATCACCTGAGGTCAGGAG |
| SEQ ID NO: 967 | CATGGGAAAAAAAAGAGAGAGAAAATAAAATTGAGTCATTTAGTGGATATAGAAAATTG |
| | AAAGGAAACCACATACTGGAAACAATTCAATATGCCTACAA |
| SEQ ID NO: 968 | GGAAAAAGAAAAGGAAAGGAAAGGAAAGGAAAGGAAAGGAAAGGAAAGGAAAGGAAA |
| | GGAAAGGAAAGAAAGAAAATTGACTCGTTTAGTGGATATAGAA |
| SEQ ID NO: 969 | CGTGGAAAAGAAACAGAAAAGAAAAGAAAAGAAAATTGACTTGTTTAGTGGATATAGAA |
| | ACTTGAAACTAGACCATGTACTGGAAACAATTCAGTATGCC |
| SEQ ID NO: 970 | GAAAAAAAATAAAAAAAAAGAAAATTGACTCGTTTAGTGTATATAGAAAGGTGAAAGAA |
| | GGCCATATACTGGAAACAATTCAGTATGTCTACAGAAATTG |
| SEQ ID NO: 971 | TTCTAAATAAATCTATATTTTTTAAAAAAAGAATTGATTGGCCGGCCATGGTGGCTCACAC |
| | CTGTAATCCAGCACTTTGGGAGGCCGAGGCAGGCGGGTC |
| SEQ ID NO: 972 | TTATTTGAGCGTTTTAAAAGTAGATGCCCTTAGCCATTACGACGGTTCGTATTTACTCTGAA |
| | ACAAGAAACACACTCAAAACTCAGAGAAAACATTGTTC |
| SEQ ID NO: 973 | CACTTTGGGAGGCCGAGGCGGGTTGATCACGAGGTCAGGAGTTCAAGACCACCCTGGCCA |
| | AGATGGTGAAACCCCATCTCCACTAAAAATACAAAAAATT |
| SEQ ID NO: 974 | ACTTTCTGGAGTTTCAAAAACAGACTGTACGCCAAGGGTCATATCTTTTTTTGTATTGGTTT |
| | GTGTCTTGGTTGGTGTCTTAGGTGTTAATCCTACAGTG |
| SEQ ID NO: 975 | ACCTGACATAACGGGGTTCAAGACTGACAACGCCTCACGCCCATCCAAAAACGTTTACAT |
| | GGCTTCCTTGTCTCTTTTTTTTTTTCTGTCCTAAAGTCGC |
| SEQ ID NO: 976 | TAGACAAGTTCTAAAAGAGCTGTAACACTGAAGATGGTTCTCTTCTCTGGAAAAACTTCCC |
| | ACGGGAAAAACAAAAATCTTCCTTTAAAAATTTTTTTAA |
| SEQ ID NO: 977 | CTGAATATTTAAAAGAAGAGAAGCAAATCCAGTCTCACATAGATAGCTGGAAAAGTGATC |
| | ATACTTGCGGAAAGGCTGGCATAAGGGCCTTTTGAGAAGT |
| SEQ ID NO: 978 | CACAAAAAGAAAAAGAAAAATCTGTCTGTACTGTCTATTGAACATAAGGCTGCAAAAAAG |
| | ATAAATTTAGACACAGTCATTGACAAATTTGCAGAAGTTA |
| SEQ ID NO: 979 | GAATGTTCTGTTACTAAAGAGAGACGTGTGGGTGGGGTGTTTCATGCTTTGGGAGGTTGGG |
| | GTAGCTCCACAAATGTCACCCAGGTTGTAGCGGAGTGGT |
| SEQ ID NO: 980 | CATCAACGGTGTTTAAAAATCAGATAGAAAGTTGTGTGTTTGTTGCGAGGTGTGAGACAAC |
| | ATTTTGTTTGAACATTTATTTTGGGCTGTTGTAATGGTG |
| SEQ ID NO: 981 | ACATTTTTAAGTATTAAAAGTTAGGCAACTACAACCAAGGAACTTGGTCATTTGTTATTTG |
| | TACCAAATGTTCACAAACTTATTCGGGCGTGGTGGTGCC |
| SEQ ID NO: 982 | CCCCACACAAAAACCAATTTAAAAACTACTTATTTAGCAGGTTTTTCCTTAGCACCTCTTCC |
| | TGTACATGACACCGTATGGGGGCTGTGGGCCTGCCCTG |
| SEQ ID NO: 983 | CAGGAAGAAACAAGACTTAAAGAAAAATGTGGCCAGGCGTGGTGGCTCACATCTGTAATC |
| | CCAGCACTTTGGGGGGCCAAGGCAGACGGATCACTTGAGG |
| SEQ ID NO: 984 | ATGAAGGGGGGGAAAAAAAGAGGAAAATCTGTTTAGTGTAGCCACCTTCATCAATGACCT |
| | TAGCTAGATCTTCAAGACAACTTGTTGCAGTTTTTACAAT |
| SEQ ID NO: 985 | TAATGCAGAAAAGAAGTTGATTTTAACACAATTGTTCATATTAAATGAGGCTAGATTTGGA |
| | TTTATATAGTTTCAGCTGCATAGCTCCTTGACTAAATGG |
| SEQ ID NO: 986 | ATGAAAAACCTCTTTTCCATGAAATTGAAAAAAAATGTTTTTGAAAAGGAAGAAAAAAAA |
| | GAAATACCTGAGGCTGGGTAATTTATAAAGAAAGGAGGTT |
| SEQ ID NO: 987 | CACATTATGTAATAAATTTTAAAAATGAAAAAATAATAAAAAGGAGAGGTGTTGGTACCA |
| | GAAGGAGGGAAGGAAAGAATGAGAATGCCAAGCAGATAAA |
| SEQ ID NO: 988 | CTCAGACATCCTTATTATCAGTCTAGTGATTTTTGCTGCTGTTCTATTCCTTTTATTTTCCTG |
| | TTAGCTCCACAATAACCTTTTAGTAGTCATTTCTCTT |
| SEQ ID NO: 989 | ATATGGAGACTGAACAAAGAAACAGAAAAAGAAATGTGGCCTCTTCCTTGGCTAAAAATT |
| | TTAACATAACGTGGTCTTGAGAAAAACATGGTCTATTTCT |
| SEQ ID NO: 990 | CCAATATTAAATTTAAAAAAATTGTTGTAAAGATTAAATGAGACAATATATGGTCAAATCT |
| | TTAGCAGAATACTTAAAATATAACAGGAGAGGCTCAATA |
| SEQ ID NO: 991 | ACAGAAAATACTTCTGTAAGGATGTCTGTCTAGCAACTGCCTGTTCAAGCTTGGACTTGTG |
| | CCACCCTTGTAGCCAAGGATAATTGATTTGAACAATTGT |
| SEQ ID NO: 992 | GTGCTTTTGTGGTGCGAATAGTAGGTGAGCCGTAAGTGTTTTTGTAATTCAGGGTGCGGGC |
| | TCGTGTTTTGTGGCTGTGTTCTGTCCGGTCAGTTGTTTC |
| SEQ ID NO: 993 | CCTAACATAAACGGGGTTCAAAACTGACATCGCCTAACGCCTACCCGAAAACGTTTACGTG |
| | GCTTCCTTGTCTCTTTGTTTTTTCTGTCCTAAAGTCGCC |
| SEQ ID NO: 994 | ACATACCTTGGCTCACCGCCGACGCGGTGACCCTTCGCCAGAGACCCTTCGCCGCCCTCCT |
| | AGGGATCTTGAGGGCCTTCCTTGTTGTTCTCACCGAAGC |
| SEQ ID NO: 995 | AATTTTCCAGTGCAAAAAGCAGTTGGTATGCAAAGGTCTACTTCTCGCTTTTTTTTGCTGTA |
| | TAATCTTGGTGGTGGTGTGTTATTCTTATAGCCGGATG |
| SEQ ID NO: 996 | TTCACAGTACTTGACTTACTCCAAGACCAGCAATAGATAGAGCCCTAGCAGATCCTTGATA |
| | ACACTGTCTGAAAAGACAGAGCCCCAGGGAATGAGTTCC |
| SEQ ID NO: 997 | ATTTTTTGTAGTTTAAAGAATAGTCTACACAGCAAGGGTTACTTGTTTTTTTTACTGGCTTG |
| | TGTTTTAGTCTTAATCGTTACTCTCACAGTCGAAGGCT |
| SEQ ID NO: 998 | CCTAACATAAACGGGGTTCAAAACTGACATCGCCTCACGCCTACCCGAAAACGTTTACGTG |
| | GCTTCCTTGTCTCTTTGTTTTTTCTGTCCTAAAGTCGCC |
| SEQ ID NO: 999 | GGAGGTTGTAGTGCAAAAAGCAGTTTGTCTACCAAGTGATACTTTCAGCTTTTACAAATGC |
| | TGAACAATATCCATGGTGTGTTTTCATGTCACCTCCTCT |
| SEQ ID NO: 1000 | ATTTCTTTTTTTCTGGTTTCAAAAATAGACCGTACGCTCCTTGTTACTGCTTTCTTTCATTGG |
| | TTTGTGTTTTTTGTGGTGCCCTTAAGTGTTACTGTTA |
| SEQ ID NO: 1001 | CCTGACATGCCTTGGCTCACCGCCGACGCGGATATCGCCGCCAGGGACCCTTCCCGCCCTC |
| | CTACGAATCTTGAGTGCGCTTCCTTGGTGTTCTCACCGA |
| SEQ ID NO: 1002 | AATTTTTGTAATGAAAAAATAGACGGCAAGGGTTATTCTTAAAACTGCAGTTTTGTAGCTT |
| | GGGTGGCATGTTAAGTGTTCTCCTTACAGTCGCAACGAT |
| SEQ ID NO: 1003 | TATTTTTGTCGTTTTAAAAATAGGTTTTGTTTGCTGCAGAAACGCATACTGGGCGTCAGCGT |
| | AAGCTGAGAATCAGGTCTTTGTTCCCATCTGTAACTTC |
| SEQ ID NO: 1004 | TCATTAGGAAAATGCTAATTAAAACTACAATGAGATATCCTTACACATCTATCAGAAAAGC |
| | TAAAATAAAAAATTGTTGCCGGTTGCAGTGGCTCATTCC |
| SEQ ID NO: 1005 | TTTTTTTTTTTTTTTTGCGGTGTGGAAAGATAGACTGCATGCAGTCTATCGTGTTATATCCTG |
| | GACGGCATCCGAAGTGTTTTTCCTTGTAGTTAGGAGG |
| SEQ ID NO: 1006 | CCTGACATGCCTTGGCTCACCGCCGATGTGGATATCGCCGCCAGGGACCCTTCCCGCCCTC |
| | CTACGAATCTTGAGTGCGCTTCCTTGGTGTTCTCACCGA |
| SEQ ID NO: 1007 | AATTTTTGTAATGAAAAAATAGACTCCCCTATAAGGGTTATTCTTAAAACTGCAGTTTTGT |
| | GGCTTGGGTGGCATGTTAAGTGTTCTCCTTACAGTCGCA |
| SEQ ID NO: 1008 | AAATCTTGCAGTGCAAAAAGTGAATTCTGCAGGAAGGGCTACTCTTAGTTTCTACTTAGCT |
| | TTCTACGGGTTTGTAGCTTTGCTGGCATGTTAAGTGTTG |
| SEQ ID NO: 1009 | AATTTTGTGCTGGAAAACGCAGATTGTATGTGAAGGGATACTTTTAGTAGTGCATAATATG |
| | GGTGGTGTCATGTTATTTTTACAGTTGGATGGCTGGGGA |
| SEQ ID NO: 1010 | TTCACAGTACTTGACTGACTCCAAGGCCAGCAATAGATAGAGCCCTAGCAGATCCTTGATA |
| | ACACTGTCTGAAAAGCCAGAGCCCTGAGGAATTAGTTCC |
| SEQ ID NO: 1011 | ATTTTTTGTAGTTTCAAGAATACGCTGTACTCTAAGGGCTACTTTTTTCTTTCATTGATTTGT |
| | GTCTTGGACGTGTTATTCTTACAGTGGAAGGCTGGGA |
| SEQ ID NO: 1012 | TCTTCTTATTCTCAGTTGAGCTTTTTCTAATTAAATGTATTACGAAATATTTTCAAGAGTCC |
| | CTTAAGAAAATTTCCACATATTTCAACATGTATCTTGC |
| SEQ ID NO: 1013 | GAGGCCGAGACAGGCCAATCATGAGGTCAGGAGATCGAGACCATCCTGGCTAACACGGTG |
| | AAACCCCCGTCTCTACCAAAAATACAAAAATTAGCTGAGT |
| SEQ ID NO: 1014 | CTGAATTTGAAAAAAAACAAAAAAAAACAAAAAAACAGCTGGGCTAGTCATAGTGGCTCA |
| | CACCTATAATCTCAGCACTTTTGGGAGGCCAAGGTGGGTG |
| SEQ ID NO: 1015 | AGATTAAAACAGGGACATAGAATTTGAAGCTTCTAGAATCTATAACTACAACAAATATTA |
| | AGTACACCCCAACTGCTAGCCAGATTAACAGAAACGCTCA |
| SEQ ID NO: 1016 | AAAATGAAAAATAAATAAAATGAAATATCAGAAACACATTTTAACTAGGCATCCAGTGCT |
| | CCACCTTTAAAACAAGGAAGTGCGCTTTCGGCTGCATTAA |
| SEQ ID NO: 1017 | ATCATGTTTTATAAAAAAAGACTTAAAGAGGAAAACATTATGGTGCAACTTTAGGCTTAAG |
| | TGATTCATTGTCACTGTTTGTTTAAACATTGTGTAACAG |
| SEQ ID NO: 1018 | TTTTTTTTTTTTTTTTTTTTTTTTTAAAGGGAGTCTCATTCTGTTGCCCAGGCTGGAGTGCAG |
| | TGGCATCATCTCAGCTCACTGCAACCTCCACCTCCCG |
| SEQ ID NO: 1019 | TTTAAAAAAAAAAAAAAAAGTTGGAGCGTAATTCTCTCCATCCTCCCAAAGTGGGCTGGA |
| | CTTAATAACTTGCTTCAAATGAATACATTGTTTTGGAAGT |
| SEQ ID NO: 1020 | TAAATAAGTTGGTGTTCAGCCTTTTACTATCAGTTCTTTGAGCTTATTAGTCTGCCTCTAAG |
| | AGACAGGCGCAAAATTAAATTTCCAAGCTTGTAGGCTG |
| SEQ ID NO: 1021 | GTCACTCTTGTCCAATGAGAGATCATAACTTGAAGTCGGTGGTCTTTATTGTATAATTTATT |
| | TATTATAAAAATGCATACAACATAAAAGCATCTTCAGC |
| SEQ ID NO: 1022 | CTGAACAACCAAAAAAACATAAAAATTTAACTCCTCCTCAGCTTCACTTTGGCACAATGCC |
| | AGAAGGACCTCTATTAAGTTGTGCACATTTGTGCAGCCC |
| SEQ ID NO: 1023 | CACCCCCAAAAATGTGTGAAATGGAAAACTGCATGTATATTTTACCACAGTGAAGAAACA |
| | AAAAAAAGAAGTTATTCCTCTGGGCTCCACTGTACCAGGG |
| SEQ ID NO: 1024 | CAACAGAAAGATCAACAGAAAGATTGATTCAAACTTTCTTGGGCATCAGAAAGTAATGAT |
| | TCAGAGTTTGGGCTCTAGAATTAGACTGCCCAGGTGCCAA |
| SEQ ID NO: 1025 | CATCAATAAATAAATAAATAAAATAAGAAAAATGAATAAAAATAAAAGTGGAGAGTTCA |
| | AACCAACCCAGTACAATCACTCTGGAACCCATCTAGAGCTT |
| SEQ ID NO: 1026 | CACAAAAAAAGAAAAATTAAAATAAATTTTAAGAGTCCTTTATCAAGAGGGCCTGCCTGC |
| | TGCCCTCCTGGCTTGTGAGCTGCATCTGTTAGCCTTGTGA |
| SEQ ID NO: 1027 | ATTGGATCGACTCCATTCTCCTCCTCCAGGTGGTGATGGCAACGAGCAACAGGAACACACT |
| | CCTTCCCCAAACACCTCACCCTCCACCTTCTCAAATTGG |
| SEQ ID NO: 1028 | AACCATTAGTGACAAAAACAGGTTTGTAATTTTGGAAACCTCAGAAGGTTGCTTCCAATTT |
| | CACAGCAATATGGCTAAAGAAGAAAAAACAGGTATGCTA |
| SEQ ID NO: 1029 | CTATTTCAGTAAAAATAAATAAATATGGTGCGCGCCTGTAGTCCCCCCTACTGGCTGGCGC |
| | GGGAGGATCGCTTGAGCCCAAGATTTCCAGCCTGAGCGA |
| SEQ ID NO: 1030 | AGAGAAATGCTTCTGGACGTTGCCAAATTCCCCCAGGGAAACAAAATTGCCCTCGGTTGA |
| | GAACCACTGAGTTACAGGGATAAAATTATCAGATGTGTTC |
| SEQ ID NO: 1031 | TCCCCAACAAATAAAGTTGTAAAGTGGAAATTGACAACTAAAGATAAATAAGAAAATCTA |
| | TAAGCAGAGTGGGGTTATTTTTAACACATCTCTCTCAGTA |
| SEQ ID NO: 1032 | AAAAATAAAATTTTTTAAAGAAAGAAAAAATAATTGATAATAATAATAAAATAATGGTTA |
| | TTCGAACTCCGTTGGTCATCAAGGCTTGTGTTGCATATGG |
| SEQ ID NO: 1033 | AACTTCCTCCTCTCCCGCAGGATTAAAAAAAAGAATTAGCTCACATAACTATAGAGGCTAG |
| | AAAGTCCCAAGATCTGCAGGGTGAGTCAACAAGCTAGAG |
| SEQ ID NO: 1034 | TTGAGTTAATGACAGATTGTTTTCTTTTGTTCCCCTAGTGTTCTACAAAGTGCTCTAATTCA |
| | CTGTTAATGAGGCTCCAGACTGCATTTCCACCATTTGT |
| SEQ ID NO: 1035 | AAAATACTTATCTATTGGGTTATCCTGCTAGACAAAAATCTTAGAAAGCTCTAACATTAAT |
| | CTAGAGTTTTTAAAAGGGCAAATTGTAGAATCTAAAGAG |
| SEQ ID NO: 1036 | TTAAAACTTCTAAACTCTTAAACAAATTATAAATACTTGATACTTTGAAGAACTGCAAAAC |
| | AAAATGTTTTTAAGTTTTCATAAGGCAAAATCATTGAGT |
| SEQ ID NO: 1037 | CACTTTGGGAAGCTGAGGCTGGCGGATCATGAGGTCAGGAGATTGAGACCATCCTGGCCA |
| | ACATGGTGAAACCCCGTCTGTACTAAAATACAAAAAATTA |
| SEQ ID NO: 1038 | GTAAAAATGAAATACTAATAATACTTCAGACTCATACTTGTCCACCAGTCGGGGAAGTGCA |
| | GGGACAACACCAGTAATGAAGGATTTCCCCCAAGTCATG |
| SEQ ID NO: 1039 | AAACTTATTTTCCAAAAATAATTTTTAAAATTTAAAAATCTGAAAAAAGAGAAGAAGAATT |
| | GTGACTTTTACAATACTTGACATTAGATGGATAGAGACT |
| SEQ ID NO: 1040 | CCTATTCCTCCACAAAAAAAAATCCCAGCTCTGTCATTAACTGCCTGTGGAGCCTGGGCCA |
| | GGTTCTTCTCTGAAGTCTCCACTGTACAAACTGTAAAAT |
| SEQ ID NO: 1041 | TCATTAAAAAGTCAGGAAACAACAGATGTTGGAGAGGTTGTGGAAAAATAGGAATGCTTT |
| | ACATCGTTGATGGGAGTGTAAATTAGTTCAACCATTGTGG |
| SEQ ID NO: 1042 | CCACCACTTTGGGGGATGTCTGCACTCCCCTCTATCCATTCCCACTCAGTTACTTCCATTTC |
| | ACTCAACAAATATCAATGGACAGCATCCCAGTGTCCCC |
| SEQ ID NO: 1043 | CTCATTTACATATCGTTGGTGGCTGTTTTTGTGCTACAAAACCACAATCAAGCAATTGGAA |
| | CAGAGAGCTCATGGCCTGCAAAGTCAAAAATATTACAAT |
| SEQ ID NO: 1044 | AGAAACAAAAAAATAAAACATGGTATGATGCAATTTGTTTATTTTTAAGGCATTTATATTA |
| | GTCTTTGCAGGGGAAAAATGCTCTGAGCTAATGAACAGT |
| SEQ ID NO: 1045 | AAATTGTGAGTGTTAAAAATTAGACCAGCCTGTCCAACATGATGAAACCCCATCTCTACTA |
| | AAAATACAAAAAACTAGCCGGGCATGATGGCGGGCGCCT |
| SEQ ID NO: 1046 | AAAAAAAAAAAAGAGCAAGCAACCCTCATAAAAAAATGAAATAACTTAAAAAAAAATAG |
| | CCACCCTGAAGATGTATAGTTACAGGTCAATGGGATAGCCA |
| SEQ ID NO: 1047 | GGCATTTTGTAAAAGAAAAGAAAAAAACACAGAGAGTACTAAGCGCACCCTGAGAGTTTC |
| | GGATTCAGAATGAGGTGGAAAAGTCAGAATTAGGTGAAAT |
| SEQ ID NO: 1048 | TTGGGGGAATAAAAAAAAAGGATTGCAGGTGAGCCCCCATATCCCTGTCATCTAGGCACT |
| | GCACTGCCCATCCCCCTGTCTGCCTGCCAGTCAGTCTTGT |
| SEQ ID NO: 1049 | GCAATTTTTTTTTAATTAAAAAAAAATTGTTATTTTAAAACAATGGACTAGTTTTTAAAATG |
| | TGGATATTTATAGTGTTTAATGATATGAGACATTAACT |
| SEQ ID NO: 1050 | GCCACATGAACATTTGTAACTGCAAGGGAGTCTGAGAAGCATAGGACTATTGCTGATTCCA |
| | GGCAAATTAGGGTTCTGTTAGTAAGGTAGAGGGGAAGAA |
| SEQ ID NO: 1051 | ACGGATTTGGAATAGAGTAAGATAGGGATATATTTATTCTCTTAGTAGCTGGTGCAAAGAT |
| | TCCTTTTTGGGGGAAACGTGTCTTTGTAAAATTACTTCA |
| SEQ ID NO: 1052 | CATGGAGAAAAAATAAAAGGTAGACACAACCAAATGTTCTTTGAGGGTCAAACGTTATAG |
| | ACCCCCGAAGAAATGTCTACCAGGAACCATTTCTGGAATA |
| SEQ ID NO: 1053 | AAAACAACAACAACAACAAAGAATCAGAAGAGATACTAGGCTATCTAATTCCTAAATCCA |
| | AACCTGATATTTCTAAGTAAGATTATAAGAATTTTTATTG |
| SEQ ID NO: 1054 | AACCTACTGCAATTTTATTAAAAGCTGATCAATACTTCAAGTAAAACTCGTTGTAAACATA |
| | GAGATTCAGACTTTGGTCCTATATCAATCAATGCACTCC |
| SEQ ID NO: 1055 | GTTTAAAAACAATCTTGAGGTTTGTTTAGGATAATAGCAATTGTTGTCTAGGAGTTAAGCT |
| | TGGAGAAGAATGGAAAGATCTTGAATGTCAGAACACAAA |
| SEQ ID NO: 1056 | GGAGAAGTAGGAGCAGAAGAAGGAGAAGGAAGGAAAGAAGAAGAACAAAGCTAGAGGT |
| | ATTACCCTACCTGATTTCAAAGCTTATAAACTCTCAGGTGTG |
| SEQ ID NO: 1057 | GTTAACAACAAAAAAAAAAAGCAGGAGAGCAAAATAAAGAGAAAAGTAGAAATCAGGCA |
| | TTCAGAAAACAAACAAAAAAAAACCAATACAATAAGTAAAT |
| SEQ ID NO: 1058 | CCCCCCAAAAGTCAATAAAAAACCAAAAACATGTTACTGCTACAGAAGTTATAGACCATG |
| | GAACACAATTTGAGGAGCACAGATTTAGGTTACTTAAAGT |
| SEQ ID NO: 1059 | TAAAAATTATTTAAAAATAAATAAATAAATAAATTGCACTCCTTATAGAAAAACAGCTTAC |
| | TGTCGCCTGTCCCAAGTGAGTGAGTTTCAGATGAATGAG |
| SEQ ID NO: 1060 | GACATACTCCAGATAAAGGCAGACATCGCTCCATGTTGGCCAATGTCAGGACTGCTTATCT |
| | CTCTCTTTTTTTATTGTTATACTTTAAGTTCTGGGATAC |
| SEQ ID NO: 1061 | ATAAAAATAAGTAAAAAAGAATAGGTTAATAAATTGTAATATATCTATAGGTATGGGATA |
| | GCATTTCTTATAAATGAAGTTTTGGAAAAAGATTTTAAGA |
| SEQ ID NO: 1062 | AGAAAAGAAAGATTGGGGAGAAAAGTACAGTGTAAATATTTCTAGAAAATTCCTGAAAAT |
| | GAAGATAATTTTATTTATTGATTTTGATGGTATGTTTGAT |
| SEQ ID NO: 1063 | AGGGGGGTGTGGGGATGAAGTATGGTTTGAATGCCACCCTCTAATGAAGCATTTCCCATCC |
| | CCTGACCCAATGTGAGCTCTCCTTTCTCTGAAACCCCGT |
| SEQ ID NO: 1064 | CACTCTAATTATAAAAATTTTCATCTAATGCTCAAGGGCTTGGAAAAGATCCTAATGGTTA |
| | TTTTATCTCCATTAAAGGCAATTGGATGTGCTTGTGCCC |
| SEQ ID NO: 1065 | AAGAAGTAAAAAAAGAAGGCTGAGCATGGTGGCTCACACCCGTAATCCCAGCACTTCGGG |
| | AGGCCAAGGCAGAAGGATAGCTTGAGCCCAGGAGTTTGAG |
| SEQ ID NO: 1066 | AAAAAAAATTGACCTAGTACTCAGATTTATATAGTGAAATCTTAAATTTTGCTATCTCGCA |
| | AAGTAAAGTTTGTCATGCTTTTTCATATTCCTCCAGGTA |
| SEQ ID NO: 1067 | AGAAAAAATAAAGAAAAACGAAAACTCTCAAATAAAGTTAATATAACAAGGATTAAAAT |
| | ACCTAAGAAAAACTGAATACAAGTTTTTGAAAAAACACCAC |
| SEQ ID NO: 1068 | CCCACTTCCCAACCACCCCCCAGCAAAAAAAAAAAAAAAAAAAAACAGGAAAAGAAAAA |
| | AAAAGCATATCCCTAGAGAACACTGGCTTTTCATCATTTTT |
| SEQ ID NO: 1069 | TTAATTTTATATCTGTTATGGTGATCTGTGATTAGTAATCCTTGATGGTACCATTGATGAAA |
| | GTCAAAATTACAACAAATTTAGATTAAAGATATGAATC |
| SEQ ID NO: 1070 | AGCACTAGATTCTTCTAGACACCTCCGCCAAAGCACTTTGCCTGTTCTCTGGGCTTGTAGG |
| | GGCTGAGAGGAAGGTGGTGATAGTGAGCTTTCCACAAAG |
| SEQ ID NO: 1071 | CTCCTATCAAAAAAAAAACCCACAAAAGACTTGAACTCTAGAATCAAGTTGGCTGATTTTC |
| | TATTCTAGGTTAGCACTTCTCGAGTGGGTAACTTTCATC |
| SEQ ID NO: 1072 | CGAATTCAACTGAGTATCCAGTTTCATACAAATAATAATAACATCTTCTATAAAATGTATT |
| | CTTGTGTATTAACACATGGGGCTCTGTAGTACTCCAGCT |
| SEQ ID NO: 1073 | CACCAAAAAAAATTTCATTAAGTTTAAAGAGATTGAAATAACTGTGTTGTCCAATGACGAT |
| | GTAATTAAATTAGAAATAAATAATACAGTAAATTTGGAA |
| SEQ ID NO: 1074 | ATATGTAAAATTTTTAAAAAATTAATTGCTTTATTTGTTGATATTTTGTCAACTAATTAAAG |
| | GATAGAAGTATATTTATTATTACAGCAGTAAAATATTA |
| SEQ ID NO: 1075 | AAAAATAGTTTCTTTTTTTAAAGGTATCTTTTCAATATTTTCAAGAAATATGATTTTATCAC |
| | TTCTATATGTAACTACTCCAGTAACAAATTAAGTGAAA |
| SEQ ID NO: 1076 | ACCACCCCTGCCAAAAAAAATTGATGAGATGATATCTCTCTCTCCTCTGAGATACTATAAC |
| | TTGGTTTGTATTTAGTAACATTTATTTTTTTTGAAGTTT |
| SEQ ID NO: 1077 | ACATTAAAAAAAAAAATCCTTTATTTTTAACTTTGCCTTCAAAATTAAGTTCAGCATAGATT |
| | GTTAAATATAATAATATCCAAATTAAATATATCCAACA |
| SEQ ID NO: 1078 | TTGGGGTATAGAATGGTTGCAACAAAAAGTAGATCAAGGCTGGGTGTGGTGGTTATGTCT |
| | GTAATCCCAACACTTTTGGAAGCCAACGTGGAAGGATCGC |
| SEQ ID NO: 1079 | CTGAATTTAAAAAATAATAATAATACATTTGTGTCTTTTTGAGCGACTAATCTTGTCATACT |
| | TGTTACAGCAGCAATAGAAAACTAATACAGCAGCAGTA |
| SEQ ID NO: 1080 | CCCAGCCAATATACACACAAAGACGATGCAACTAATCCTCTCAGAAAGCAATCTCAAGTTT |
| | ATAGAACAAATCTCTCTCTTTACATGTATTCTTTTCCTT |
| SEQ ID NO: 1081 | TAAAGAAGAAAAAAAAGCCAGTGGGACTATTTAGAATCTACTTATAATGTGTTTCAAAGC |
| | ACTGTGTTTTGACCATCTTTCAGATCAAGAAGGATAAAAA |
| SEQ ID NO: 1082 | AACACACACACACACACACACACACACACACAAACACACACACACACACACAAAAGAAC |
| | CCCTCTTCGATCAACACATGTCTGACATAGGTGGAGTCTTT |
| SEQ ID NO: 1083 | ATGCAAAAAAAAAAAAAATTAGGGGTCCAAATACAATGCCTCAGGTCTGTAATCCCAGCA |
| | TTCTGGTAGGCCAAGGTGAGAGGATTGCTCCAGGCCAAGA |
| SEQ ID NO: 1084 | CAGGATATTTTTATTTTTAATCCTTTAATTTAAAGTGCTCAGCATGCCAAAGTGCCATACTT |
| | TGAAGTATCACTTTCTGAGCCCCAATAGAAGTATAATA |
| SEQ ID NO: 1085 | TAGTAAAACACACAGATCTTAAATATATAATTCAATGGCTTTTTACAAATATCTATCTGTA |
| | GCCAACACCCCAATCAAGGCAGAAAACATTGCCACCATT |
| SEQ ID NO: 1086 | GAAATAAAAAATAAAAAGAGCATAGTACTCTTACATGCTGTCTACAAAACAAACCCATTG |
| | TACCTATAAAGGCAAAATATGCTAAAAGTGATAAAGAAGG |
| SEQ ID NO: 1087 | TCTGGGGGAAATTTTTTTTAAATGCTAGCAAGAGATTACAAGTGAAATATGATTCATACCT |
| | ATAAATAACTGCTTTAGAAAGGGGCCAGTCTTGTTTTTA |
| SEQ ID NO: 1088 | CCCCCACAAAGAATAACATTAATAAAAAAGTAAGAAAATGTAACATAAATAGAAAAGAA |
| | CTGGCTTATTTACCCAGGTAACTACTCAATCCACAATGAAC |
| SEQ ID NO: 1089 | CTATGCAATCTATGCAGGTAACAAAACTACACTTGTACCTTATAAATTCACACAAATGAAA |
| | GAAGACAACATGAATAAAAAGGAATGAAAAGAAACAAAA |
| SEQ ID NO: 1090 | AAGGCCATCCTGCTGTGCAGATAGGAGATGAGTGAGGCCGGAGGAGTGGCAGGAGGAGG |
| | TGCCTAGGCCGGTCCTCATAGCTTTGTCCAGATAGAAGGAA |
| SEQ ID NO: 1091 | TATCACTATAAAATCTGAATAAGAAAGCAAGTATTAGTAAAAATGGGAGAAAACTTCCTT |
| | TGAAAAAAAGTTGGTAGCTCTAAAGAAAACAAATTTTAGT |
| SEQ ID NO: 1092 | AAAACATTAATAATAAAACTAGTTTTCTAAAGAACAGCTACTCGTTTGCTATCTCTTCTAGT |
| | AGACGTAGGGAGTTCAAACTGTTTTCACCATTACTGCT |
| SEQ ID NO: 1093 | CTGCTACCAAAAAAAAAAGATTGAAAATATGTTCGAAGAACTAAAGAAAATTATGCACAA |
| | AGAATTAAAAGAAAATATAGAATGATGTTTACAATGACAA |
| SEQ ID NO: 1094 | CCGATTTTTTAAAAAAGAAGAAAATCATTTCTAGAAAGTATCAAGATAATAAAATGATTTG |
| | GAAATTTTAAAAATATACCAGAAAAATTAGAATATGTAA |
| SEQ ID NO: 1095 | AAAAAAAGTTTTCATATAGATTTGGTAGAGATTGTTATACGTTGTCCCAAATAAAACTGTG |
| | TCTTTATGTTTTTAAATAAAACATTTCTTAGTTAGCGTA |
| SEQ ID NO: 1096 | TATGGAGACTGAATATTTCAGTAAAAAAAATTATATATATATATATATATATAAAATAAAT |
| | AAATAAAATACTAATTGGATTGTTTGAAACACAAAGAAT |
| SEQ ID NO: 1097 | CCCCACATGCTATAAACTAAAAAAAAGAGGCTGGGCGTGGTGGCTTATGCTTGTAATCCCA |
| | GCACTCTGGGAGGCTGAGGTGGGTTGATCACTTGAGGTC |
| SEQ ID NO: 1098 | CCAAGATTTAAAAAAAGACAAAGAAAGAGGCAAACATAGAATCTAGGGAAGCATGTCTCT |
| | AACAAAGGAGAGTAGCAAAAGAAGTGCCTGGCTGAAAACC |
| SEQ ID NO: 1099 | CTGAACAAAAAAATAAATAAAAAATAAAAATAAAATTGGTTTGATAGGGTCACTCACACC |
| | TGTAATCCCAGCACATTGGAATGTCAAGGTGGGAGATCGC |
| SEQ ID NO: 1100 | CTGAACAAAAGAAAATAAATAAATAAGAAGAAGAAGATATGGCCAAGAGAAAATATATT |
| | ATATTCAGAGCATATTATGAGAGTCATTTTTCTGGGTACAT |
| SEQ ID NO: 1101 | ACACACACACAAACACACACACACACACACACACACACACACACACAGAGATAGATAAT |
| | ACAGAAGCTGTGAGAGGAGATAAAGATTACTTAGTTTACAT |
| SEQ ID NO: 1102 | AGGAGTATCACTTGCATCCAGGAGTTCAAGACCAGCCTGGACAACGTAGTGAGACCCCAT |
| | GTCTACACAAAATTAAAAAATTAACCACGTGTGGTAGTGC |
| SEQ ID NO: 1103 | AAAAGAAATTAAAAAGTAAAAACAAAAAAAAAAGAAAAGAAGATAAAACAAAAGCAAA |
| | TAAAAGAAAAAAGAAAAGTTACCTGAAATGAATTGCAGCCTA |
| SEQ ID NO: 1104 | CTGAAGTTTGTTGGAATAAAAAAGAAAAATGGATAAAAGGTTTTTAAAACAGAAAGGGGA |
| | AAAAAACAGGCCTAAGAGAGGGGTCTCCCCCAGATCTCAG |
| SEQ ID NO: 1105 | CTCTCCTCTACACATAAAGAAATATAAGATATAATTTAAAATATAACAAAGAAGAGACTA |
| | TCAATTTAACCAGTGAGTTTGAAAAATAACTTAATAAAAT |
| SEQ ID NO: 1106 | ATGAAAAACAGCTTTCCCATACACACACAGTTATTTACATATTACTGTATTAAAAAGTACA |
| | TACAGGCTGGGCACGGTGGCTTACGCCTGTAATCCCAGC |
| SEQ ID NO: 1107 | TAAAACTAGTACCTCAATTACCCATGTATCCATACACTTGAAACTATACCCATTAAACACT |
| | AACTCCCTGGCCGGGCACGGTGGCTCCTGCCTGTAATCC |
| SEQ ID NO: 1108 | AAAAAAAATTAACATGTTAAATTTCAGGAAAGGTATCTCTCTATTAGCCCCCACTTATTCT |
| | TTCATTTATTCATTCTGCATTTATTTAGCATTATCATTT |
| SEQ ID NO: 1109 | CAGTAAAAAACAGAAAGAAAAAATTATCCTAAAATTGGCTGTGGTAATGGTTGCGCATAT |
| | GCTGTGAATAGGCTTCCAAATATTGAAATGTCCACTTCAA |
| SEQ ID NO: 1110 | TGTGCAGATAACAAGAGTAGCTTGATTCATAATCACCAAAAACTAGAAACAATCTAAATG |
| | CCCTTTGTTGCTGAATGAATAAACAAACTGTGGTACATTC |
| SEQ ID NO: 1111 | GTCTTCATGGTTGAAGAACAGGTCCAGGCCGGGCTCGGCAGGTCACGCCTGTAATCCCAGC |
| | ACTTTGGGAGGCTGAAGCAGGTGGATCACCTGAGGTCAG |
| SEQ ID NO: 1112 | CTGAATATAATTTTTTAAAAAAATAAATAAAAGTAAAAACTACAAATCACATTAAATGCA |
| | GGTATCACTTATATACTCCAATTACTCTATTGTTCTCCAA |
| SEQ ID NO: 1113 | CCCATGTGGAGAGAGGCTGAGAAACAATCAAAATACACATTCTAAAACTCCCAACGATTA |
| | GAGATTGTTTTTTATTACTAGTTCAGTTTTGATGATATGA |
| SEQ ID NO: 1114 | AAGGAAAGAAAGAAAGAAAGAAAAGGAAAGGAAAGGAAAGGAGGAAGGAAGGAAGGA |
| | GAAAGAAATAAAGAAAGAAAGAAAGAAAGAAAGAAAAAGAAAA |
| SEQ ID NO: 1115 | AGGAATTTTAAAAAATACATTATTGACTTACTTTTCTTTTAAACATAACAAAGTACTGCAT |
| | AATATTAAATTAGACCTTTTGATCTGAAATGTAACTATT |
| SEQ ID NO: 1116 | TTGAAAAAAATAAAAAAGAAAGAAAGAGAGGACTAAATAATGAGAGCACACCTTAGCCA |
| | TTGTTTGTCTACTTTGACTAAGAGCATAGTAGAAATGCATT |
| SEQ ID NO: 1117 | CCAAAAAAAAAAAAAGCAGGCCTAGAATATGGAATATATCAGTCAAGATAGGCCAAGTTA |
| | TGCAGCAGAAACAATTGGAAATAATAGCAGTGAAGCACAG |
| SEQ ID NO: 1118 | ACTCCTTGTCTAAATCAGAGGTTGCAGCTCTGGTCATGTTATATTAGATGATTCCGCACTCT |
| | AAACCTCCGTAGTAGATCCTGCCCCACCTCAGGGCTAT |
| SEQ ID NO: 1119 | CTGAACTTTGTTAGTAAAAAAATATATATTAAAAAACAACAACAACAACAACAAAAACAA |
| | CTCGGGTGCGGTGGCTCTCGCCTGTAATCCCAGCACTTTG |
| SEQ ID NO: 1120 | ATGCTATTAAATTTCACTGGAAAGCCCCCTATGCTATTAAATTTCTTTAAATGAAAAATAA |
| | AAAACAATTGAGTCAAATAAATTAAGAAAAAATATTCTA |
| SEQ ID NO: 1121 | CTTGCAATAATCATTTTTAAATGCTAAAAGAATATACATTTATGGTAAAAGAATAAAATTC |
| | CATTTTAAAAGCTTTCGTAAACTAAATAATTTTTTGTAA |
| SEQ ID NO: 1122 | TTATATTTAAACAGAGGAAACTGGGCTACATGATTTCTTCATTCCCTCTAAGAAAGAAATG |
| | TTTTGCCTACCTCACTTCTCATATTTTGGAAATGAACAT |
| SEQ ID NO: 1123 | TTTCCTTTTCCTAATCACCATGCTTCTGCCAGCAACTTACCAACTCAATGAATGCCTCATCC |
| | ACTGCCACAACGCCAGACACCCACATTGCTTCTGATCA |
| SEQ ID NO: 1124 | TGGCTGCTGTATTACTCTGTCACATGTCTTGGGAGAGGTCCAGTTTCTGTCACCCTAGCATT |
| | AACTGAAAAGCGGGATGAAATATGAAGACATTTTGCAG |
| SEQ ID NO: 1125 | AGAAACAAATACAAATAAATACAATAGGCAAAATACACTTTTATATTATTTAAAGTTTTTA |
| | AAATGGAAAAAGAAAAGCATTTGGCCCTTTGAGTATAGC |
| SEQ ID NO: 1126 | AAATGAGTGAATAAATAGAGAATACTCCTTATGTTTTAAAATCCTCAGATCAAAGAAGTG |
| | ATCTTTCTACTGTGGGCTTCATTCAGCCTCCGGACAGGCA |
| SEQ ID NO: 1127 | TAAGAAAAAATTTTTTTCAATTGGTAATTTAAAACTGATTAATACCACTATGCAACTGAGA |
| | GAGTGAAAATGTCTGTTTCCTGTCAATTTTTCAACTGAA |
| SEQ ID NO: 1128 | GAGAGACAGAGAACAAAAGAAAAGAAAGAAAGTAAGAAAAAAAGAAAGAAAGAGAGAG |
| | AGAGAAAGAAAGAAGGAAATAAAGAAAGAGAGAGAAAGAAAA |
| SEQ ID NO: 1129 | GCATCCTCCCTGAGGGATTTTTTTTTTAAGTATACAATTCAGTGATTTTTTAAAAGTATATT |
| | CATGAAGTTATGCAACCATTGTCACTATCTAATTGCAG |
| SEQ ID NO: 1130 | AGGAAAAAAATCTAGGTACATAAACCCTGGGACCTTGCTTTCTAAATTTGCCATCTAACAT |
| | CTGATTTTGACCCTCAGGGTTGTAGTGTGAAATGATTTG |
| SEQ ID NO: 1131 | TTGGGGAATAATAACATTTTAAAAAATTACTTGTAAAAATATTTTTTAAAGATGTTAATTG |
| | AAATCCCTAGGGCAACCACTAAGAAAATAACTTAGAAAT |
| SEQ ID NO: 1132 | TTTGGTTGCCTTGACTCCTGATTCATGTCTTTACGTTCTCTTCCTCAACAAGCTTCTTAATAG |
| | GTATTTTCTTCCAAAATAGGTTTTTTTTTTTGTTTTT |
| SEQ ID NO: 1133 | AAAAAAAAAAAAAAAAAATTAGCCAGGCGTGGTGGTGTGTGCCTGTAATCCCAGCTACTT |
| | GGGAGGCTAAGACAGGAGAATCACTTGAACCCAGGAAGCA |
| SEQ ID NO: 1134 | AACCACCCCGATGCGAATCAGGATATTGCCAGCATCCCAGAAGCCCAGCCCCCTGAGCCC |
| | CTCCCAGCCCTGTCAGTCACTCCGATCCCAGCCCTTCTGG |
| SEQ ID NO: 1135 | GCCTCCCAGGTTCAAGCAATTCTCTTGCCTCAGCCTCTCAAGCTGGGATTACAGGTGCCAG |
| | ACACGTTGCCTGGGTAATTTTTTTGTATTTTTAATAGAG |
| SEQ ID NO: 1136 | TGAGAAATTTATATTAAAAAGAAAAAGAAAAAATAAAACATGCCTGTGACAATTAATGTA |
| | GTCTTCTACACTTGATCTTAGCCAAAACCTGAGAAGCAAT |
| SEQ ID NO: 1137 | CAAAAAAAAGGGAATTGTATGAGGTGATAGATATGTTAAGTAGTTTGTGGCAATCATTTCA |
| | CAATGTATACATATATAAAACCATCACCTTGTACACTTT |
| SEQ ID NO: 1138 | GTGGAGGGTCCTGTCCCTGGGGAGACCAAGCTGGCAGGTGCTTTTTTTCTTCCTCTTGGCCT |
| | CTCATCAGCAGCCCTGTTTAAACCTTCTTGGATGGTCT |
| SEQ ID NO: 1139 | TACAAAAATATGTAGGTCTATTTCATTTTCACTACAATATAGTATTCCATTTATGTAATTTC |
| | TTTTTTTGACACAGAGTCTTGCTCTGTTGCCCAGGCTG |
| SEQ ID NO: 1140 | CCGGGGATACAAAAAAAAAAAAAAAAGAATTAAAAGAAATATCACCTAAATAATGCTTTT |
| | CTGTGTATTTACTAGTATATTTTCAGAATACCATTAAATA |
| SEQ ID NO: 1141 | ACTTGTTGTGTAAATTAGACAGTTGTGACTTTTTTTTTTTTTTTGAGACGGAGTCTTGCTCTG |
| | TCAACAGGCTGGAGTGCAGTGGTGCGATCTCAGCTCA |
| SEQ ID NO: 1142 | GAATACCATGCAGGACATAGGCATGGGCAAAGACTTCATGTCTAAGACACCAAAAACAAT |
| | GACAACAAAAGTCACAATTGACAAATGGGATCTAATTAAA |
| SEQ ID NO: 1143 | CATTGAGTTAAAAATGGACCTTTTTTATTTTTAAAAAGCTAATTAACTAGATTTGCTTACTT |
| | AAAATCAAAGCAATCAACTCGTTACAAGTCTAGAAATA |
| SEQ ID NO: 1144 | ATAGAAAACATAAATTAAAAATGTAAAAACTAAGTAATTTATATGACATAAAGTGAAAAG |
| | TACACAATTCTGTGGTATTTGGCGCATGATGTTGTCATGC |
| SEQ ID NO: 1145 | TAATAAAAATTAAAAAATAAAAGTTACACAAGTGATTTACAATAAGTTTAATTTAGGAAAT |
| | ATCAGTATTTTATGATTCCCAGCTGTTATCTCAAACATG |
| SEQ ID NO: 1146 | TATCTTTAAAAAAGAGCAAGGTACGGGCCAGGCGCAGTGGCTCACGCCTGTAATCCCAGC |
| | ACTTTGGGAGGCCGAGGCGGGCAGATCACGAGGTCAGATC |
| SEQ ID NO: 1147 | CTTCGGGGAGAGAACAACCGTTGTTTAATGGAAGATTTCGATCAGTTAGGGTACAAGCTA |
| | AATAGTTATGTTCTTGTTGTTTGAGTTGGATTAGGTGTTT |
| SEQ ID NO: 1148 | GGCATTAAAATCACTTGAACCCAGGAGGTGGAGGTTGCAGTGGGCTGAGATCACGCCACC |
| | ACACTCCATCCTGGCCAACAGAGCAGGACTCTGTCTCCAA |
| SEQ ID NO: 1149 | CTCGCAAAAAATGAACCTGTCAAAAATAAGAAGTACAACAACTTTCCAAGATACAGACAA |
| | TATAATAAGATATAAATAGAAAAAGCAAAAAGGTAAAAAG |
| SEQ ID NO: 1150 | AAAAAGTGTGGGCTACCTTTTATCACTGACCTTTGCTTGAGTCCCTTTCTAGACATTATTAG |
| | ATGGCAACAAAGAATGGAAAAGCACAAATTAAACTGAT |
| SEQ ID NO: 1151 | CGCCTCAGCCTCCCGAGTAGCTGGGATTACAGGCGCCTGCCACCGCGCCTGGCTAATTTTT |
| | TTTTTGTATTTTTAGTAGAGACGGGGGTTTCACCATGTT |
| SEQ ID NO: 1152 | TGGTCTGTAGTTAAAAAAAAAAAAATCAATGTCACAAAGGACAAGGAAATATGCAACATG |
| | TAATCTTAGATTAGATCCTGGATGTTATTAGAACAACAGG |
| SEQ ID NO: 1153 | CTCCCATGAGAAAACAACCTACAATTGGGAAAATACATAACTTAGAAAGAAAGGGAAAGT |
| | CTTTAGTGTGATAGAAATTCCTCCCATATCTGTTTCCTGT |
| SEQ ID NO: 1154 | GTTACAAAAAACAAAACAAAACAAAACAAAACAACAAACAGAAAAAAAAAGAGAGAAA |
| | AGATGCTAGAGGAAGCAGTGTGACAGACAGATCATGAGATGC |
| SEQ ID NO: 1155 | CTGAATATAATATTGTGTATATATATATTACATATAGCCTTGTAATAATTAAAGACATAGT |
| | AAATACAGCTTCTTTAAACTTCTATTCTCTCTTCTCTCT |
| SEQ ID NO: 1156 | GACTTTTAATCCATTTTTTGGCATTAATACTTTTTTCTGCTGCAAATCTGTTTAATGATGCTA |
| | ATTTGCTTAAATCATGGAATTATTTTTACATTTATAA |
| SEQ ID NO: 1157 | AGTTTTGCTCTGTTGCCCAGGCTGGAGTGCAGTGGTGCGATCTTGGCTCACTGCAACCTCT |
| | GCCTCTTGGGTTCAAGCCATTCTTCTGTCTCAGCCCCCC |
| SEQ ID NO: 1158 | ACCCCCACCCCCACCAAAAAAAAAAAAAAAAAAAAAAGATAGTTACAAATGTTTCCCAGA |
| | ATAGTTGTCCCAATCGAATCTATTTTCTCATGTGTAGTGT |
| SEQ ID NO: 1159 | CTGAACAAAAACAAAACAAAACAAAACAAAAAAACAAAGTCACATATTAGGGAAAAGGG |
| | GGACTCTCAGGAGATTAGTGCCACCCTTAAAGGAAGCAGGA |
| SEQ ID NO: 1160 | AAAAAGAAAAAAGAAAAAAATATAAATATATATATATTTTACCTATATGTTACTAGAATAT |
| | ATATTTTATATAATATATATTTTAAAATATATCTTTATA |
| SEQ ID NO: 1161 | AGTAAAAAACAAAAAAGAAAGGAAAGGAAAAAAAAAAGAAAAGCCCAGACCAGATAGC |
| | TTCATGGGTGAATTCTACCAAGCATTTAAAACTGAAGAGAAA |
| SEQ ID NO: 1162 | TGAATAAAGTAAAAAAAGAAAGAAATATAGTGAATTTTTAGCATGCAACTATTGTACATG |
| | ATGAGGGTCCATTGAGTCTAAGGGAGACCCTACGAGTGAG |
| SEQ ID NO: 1163 | TAATAAATACACACTTTTATACAGTTTCATTGGTTTGGAGGACTTTATGATGACAAGCAAA |
| | TTCAAAATCATTACACTGAAGACTCACAGAGCCAAGTAG |
| SEQ ID NO: 1164 | CATGGGAAAAAAAAATAGCGAGGAATCATATTTTCCCAGACAATTTCTTATGAGAGCCAG |
| | ATAAGATTATGCATATCAGCTCATGGTAAAAGGCAGTAAA |
| SEQ ID NO: 1165 | CTGGAACAACAACATACACATTATAAAGCTTATTGTGCTATTTCTTTAGCTATTATTTCTGT |
| | TTCTGTGAAACTTTTAAAATATTCTAAAATAATCTAGG |
| SEQ ID NO: 1166 | AACTGAAAAAAAAACAACAACAAAAAACGAAGGCAATAAGGAAATAAGGGGAGGAACT |
| | GAAGAACAGAAAAAGGAGCAGAGATAACATCCTTTAAATTAA |
| SEQ ID NO: 1167 | AGAAAAGAAAACAACAGGTACAAATATATGGATGTGGGAAGCAGCACTGTGTGTTTAACG |
| | AACTACAATTGGTTATGTATTGCTAGGGTACAAAGTGCTA |
| SEQ ID NO: 1168 | CTAAACTTTGTTGGTAAATAAAAGTTGAAAAAAAAATGACTAGGTGCAGCGGCTCACATG |
| | TCTAATCCCAGCGCTTTGGGAGGCCGAGGTGGGAGAATCA |
| SEQ ID NO: 1169 | ATGCAGAAGTAGTAAATACTGATTCATGTAAAATAATAAACAACTTTATCTTTCAGTTTTT |
| | AAAAGACAGGGTCTTGTAACGTTGCCCAGACTGGCCTTT |
| SEQ ID NO: 1170 | CTGAATTTTTAAAAGTCTAAAGAAAAGGGTTTTAGGAAGTTGTATTATAGCAGGTTGTGTC |
| | TTATGTTTGGTTTGATAAGTTATGAGCTGTTCCTATGAT |
| SEQ ID NO: 1171 | CTGAATTTTCTTGCAGTTGAACAACAGAGGCTTTTTTTGTGTGTGTGGGGGTGCTTGGTTTT |
| | GGGAGGTTGAAGAGTACTTGTTCGCAAACTCTCTAAAT |
| SEQ ID NO: 1172 | GTGGAAAACCTCTTTTCCATGAAAATAAAAAGGGATATAGAGCAAATGCAGTTCTCAATTC |
| | CTGGTACTGGGAATGTGAAGTCATTTCGCCACTTTGGAA |
| SEQ ID NO: 1173 | CTGAAAAGAAAGAGAGAGATAAAATAGGTGGGGTGCAGTGACTCACACCTGTAATCTCAA |
| | CAGTCTGGTCGGCTGAGGCAGGAGGATTGCTTGAGCTTAT |
| SEQ ID NO: 1174 | TAGTAAAAAAAAGAAAAGAATCTATAATCAAACAAGACCAAGAAACACTAGGCTAAATG |
| | AAGTTAAAAGGTCACTTTGTAGTAGAAATCATTGCAACCTT |
| SEQ ID NO: 1175 | TGGGTGTTAAAACACGGTGTTACTGGCCGGGCACGGTGGATCCCACCTGTAATCCCAGCAC |
| | TTTGGGAGTCTGAGGCAGGTGGATCACAAGGTCAGGAGT |
| SEQ ID NO: 1176 | AAAAAAAAGGATTAAATCATGATTAAAACAAGTGTGAAGAGGAGAGATAACAATTTGGG |
| | GGTTGTTTTTCCTTTATTGTCTACTTGAATTCTTGGATAAT |
| SEQ ID NO: 1177 | GATGTCAGTTTTTTTTTTTTCTAAAAGCAAACAAAAACAATTGGCTTCAGACATTTTGAACA |
| | AAACAAGCAGAAGCTGTTTTCTTTAAGAAATTACAAGC |
| SEQ ID NO: 1178 | GTGTTTAAAATTAAAAAAAAAAAAACTTTATTAAAGGCACAGAACATTAATAAAAATTGA |
| | CAATAAACTGGGCTATTAAGTAAATTGCAACAATTTCCAG |
| SEQ ID NO: 1179 | CTTCCCCTCCAAAAAAAAAAAAAAAGAAAAAGTTGAAGAATTAAGAGAAATAACACGTGT |
| | AAAATAATTTTAAATAAAATGAAAAGAGTAGTAAAAGTAT |
| SEQ ID NO: 1180 | CCTTGAAAAAAAATTTTTTTTTTTTTGAGACAGAGTCTCACTCTGTCGCCCAGGCTGGAGTG |
| | CAGTGGTGCCATCTCTGCTCACTGCAAGCTCCACCTCC |
| SEQ ID NO: 1181 | ATAAACATAAAAAACAGAAGTACTTTCCAGAGACTAGGATTACAGACAACCTGGATATAA |
| | ACTAAGTTCTGGAGGTATTTTAAAAAGAGTATTCTAAAGA |
| SEQ ID NO: 1182 | AGTTTTAAATCCACAACCACCCCACCCCAAAACAATGATTAGCACAATGAGAAACTTTACC |
| | TGAGGTTGGTTACACCCCAGATCCTTTCTTTTACACAAA |
| SEQ ID NO: 1183 | GGGAAAAAAAAAAAACAAAAAAACTTATATGGAGTTTAAAATATAATTAATTAAAAATCA |
| | ATCCTGTGGGATCACATATCTAGGGGAGACTTTAACCCAT |
| SEQ ID NO: 1184 | GTGAACTGTAAAAAAAAAGAAAAAGAAAAGAAAGACACCATTGTTTTGGATTTCAAGTTC |
| | ACTCTAAACCCAGGATGACTTATTGTGAGATCCTTAGTGA |
| SEQ ID NO: 1185 | CTCCCCTGACATAAATGGTTTTTTTTTTCCTTTTTTGAGAGGAGTCTCGCTCTGTCACCCAG |
| | GCTGGAGTGCAGTGGCACCAACTCGGCTCACTGCAAAC |
| SEQ ID NO: 1186 | CCACGGGGGGACAGAAAATAATAGATTATAGTGATCCTTCTCTTTCTGTTCATCACCATAG |
| | CAATTCATTTCTTTGATTAATCTTAGGTATGTACCATGT |
| SEQ ID NO: 1187 | GATATGGTTTGGCTCTGTGTCCCCACCCAAATTTCATCTTGAATTGTAATCCCCATGTGTCA |
| | AGGGAGGGATCTGGTGGGAGGTGACTGGATCATGGGGG |
| SEQ ID NO: 1188 | CCCAGCCAAAAGTAAAAGAAAAAAAAAGCTGCAATAGTTCTTTATATAGTTTAGATACAA |
| | GGCCCTTATCAGATATTTGATTTTCAAATATTGTCTCCCA |
| SEQ ID NO: 1189 | ACCCTCAAAAAAATTAAAAAAATAAAATAAAGAAGGGAGAACTGAACACCAGATGGGCC |
| | AAGGCTAAAAATTTCTGCCCTGGGAGTTCCAGACCATTCTG |
| SEQ ID NO: 1190 | TTTCCCCTGGGGGAGAAAAAAGAAAGGGGAACCCTCATGGTGCCCACATGCCTGTCAGGG |
| | GGAAGTCTGCTCGGGTCATCAACTATGAGGAGTTCAAGAA |
| SEQ ID NO: 1191 | CCTGACTAGCTCTAGTCTACTCCTTGTTGACTAGTTTTAACTAGCAGGAAAATAAACATCA |
| | AGAGAAACCAAGTCCTTACTGTCAGAGCTCCACACAGAG |
| SEQ ID NO: 1192 | CTCACCCACTGCCACCCAAAAGGTGTATGTGTGATCAATGAAAAGTAAGAATCAATGGTA |
| | ATACTTTTCTGTTTGAAAACACTGGAGAAATTATCAGATG |
| SEQ ID NO: 1193 | AAAAACAAAAAAATAAACAAGTTTCTCCCCTCGTTCTATGGTTTTATAGCCCCAGCAGTAT |
| | TATTTCCCAATCCTTCTAAAAAAGAAACCATAGCAACAA |
| SEQ ID NO: 1194 | TCTCCCCAGCAATACAAATAAATAAGTAGGCTGGGCACAGTGGCCCATGCCTGTAAGCAC |
| | TTTGGGAGGCCAGGGCAGTAAGATTGCTTGAGGCCAGGAG |
| SEQ ID NO: 1195 | ATCCCTCCTCGAAAAATAAAAAATGAAAAAATGGTTTTATAAAGCAAGTAAGTCATATTCT |
| | AAACAACTATGTTCACAGGTTAGCTCATTAAAGTCAGTG |
| SEQ ID NO: 1196 | CAAAAAAATTTTTTTTAAATAGTCCGGGCATAGTGGCTGACACTTGTAATCCCAACAGCTG |
| | GGGAGGCCAAGGCCGGAGGATTGCTTGAACCCAGGAATT |
| SEQ ID NO: 1197 | CCGGGGATACAACGTGTTTCCTAAAAGTAGAGGGAGGTAAGAGACGGTAGCACCTGCGGG |
| | GCGGCTTGCACGCCGAGTGCCTGTGACGCGCCGGCTTAAC |
| SEQ ID NO: 1198 | CCGGGGATACAACGTGTTTCCTAAAAGTAGAGGGAGGTAAGAGACGGTAGCACCTGCGGG |
| | GCGGCTTGCACGCCGAGTGCCTGTGACGCGCCGGCTTGAC |
| SEQ ID NO: 1199 | CCTGGGATACAACCTGTTTGCAAGGTTAGAAAGAAAAGACTGCGCCGGGTAGTTTAGGAT |
| | AGTTGGTAGGTTTTCTTACTCCTTTAAGTATCATAAGGTT |
| SEQ ID NO: 1200 | CACCCACCCACCCTCCCAAAAAAAAGGAAAAAAAAAAAGTCAAGTGGGTTGTTTTTTCAG |
| | AGATGCTACCAAAAATGTAAAAAGGTAGAATAGCCCCCAG |
| SEQ ID NO: 1201 | AAATTAAAAATAAAAAAAAAAAAAATAGGCTGGGCTCACGCCTATAATCCTACCACTTTG |
| | GGAGGCCAAGGCGGGCGGACTGCCTGAGCTCAGGAGTTCA |
| SEQ ID NO: 1202 | TTTACAAAAAAGATAAACTTGTTATATGCAGGAGACAGGAAATTGGAGGAGGGACCCAAG |
| | AAGGCCATGTCCTGGAGAATACAGCTTTCCTGGGGGCCTT |
| SEQ ID NO: 1203 | GTTGTGGTAGTTGAAATGCAAGTTTTGTTAGTTGTGTATTAGCTTTTGCTTTTTTTTTTTTTT |
| | TTTTTTTTTTTTTTTGCCGTGGAAGGTTTGTTTCACT |
| SEQ ID NO: 1204 | TGTTATTCTTTGTCAAATGGGAAGTAGCCAATGTGATTGTCTGTGGCCGTGTTTGGCTTCTC |
| | CTTGCTTGTTTCTTGTGAACTGTGTCTTTAAATTTCCA |
| SEQ ID NO: 1205 | GATCTGGCTTGTTTATAAAAGGCGTACGTTGTATCTTTGCTTCGTAGGTTTTCCTGTGTTAT |
| | ATTGTAACCTCCTGTTTTGGAATAGCGAGAGATTGATG |
| SEQ ID NO: 1206 | CTGGGCTCAAGCAATCCTCCCACCTCAGTCCGCCAAGTGCCTGGAACTATAGGCACACACC |
| | ACCACACCCAGCTAATTTTTGTATTTTTAGTGGAGATGG |
| SEQ ID NO: 1207 | CTGAACAAAAAAAAGAAAAAAATAACATTTACAAAGAATTTTAATGACACAGATTATACA |
| | TATAATGTACATATTATATATAATATATATCTTACATATA |
| SEQ ID NO: 1208 | TCCCAAAAAAAAAAAAAAAAAAAAGGTAGTCCTGTCCTCAGGAGAATCCTCATAGTACTA |
| | TAAATCAGAAAGTATTATTTCCACTTTAACAGATAAGGAA |
| SEQ ID NO: 1209 | ATTAAAAAAGGAACAAAGTATTTAATTACCTCATAGTTCTATAAGAAATTAGGTATCATTA |
| | AATATTATATAATATTCATAGCTGTTTTTATCCTTTTGT |
| SEQ ID NO: 1210 | TTTCCATTTTTTTTTTTTTTTTTTTGAGACGGAGTCTCGCTCTGTCACCCAGGCTGGAGTGCA |
| | GTGGCATGACCTCGGCTCTCTACAACCTCTGCCTCCT |
| SEQ ID NO: 1211 | TCATTTTTTGTCCTAAGAGATCATAGTGTGAAGTTTATATTATTAAAAACAAATAAATAAT |
| | AAGAAAATAGAGGCATGTAGCTTTACATGAGTTCATGTC |
| SEQ ID NO: 1212 | AAATGAAAAGTTTAAAAAAAAATAAAAGAATTAATTGCTCTTTCCTCTGATCAGAGCGCTT |
| | CACTCATCCCACTGGTGTGCACTCTCTGCATGATATTAT |
| SEQ ID NO: 1213 | AAACCAAAACCAAAACCATGTCATGCCTCTTAGCTGAGCTAAGGAAATGCATTATATGACT |
| | GTGTCCATAAACGTGTCTCTTGGCCAAGAAGCTTCCTTC |
| SEQ ID NO: 1214 | CATCGATTTTTTAAAAATCTGCTTTCATTATTTGCAGCAAGGCAAGAATCTTCTATTCAGAG |
| | CAGATGTACAATCATGGAGAGAATCCTCTGCATGGCGG |
| SEQ ID NO: 1215 | ATGTTTATTAAAAAGAAAAAATTTTTGATTCAAAGTAAATAAAGTAAAATGCACAAAGGA |
| | ACAGCCCATACCTCACCCACACATGGACAAAATAAAAGGC |
| SEQ ID NO: 1216 | AATATAGTAAAGATGTCAATTCTCCCCCAAACTGATATGCAGGTTTAATGCAATTCCTATC |
| | AAAATCCCAGCAAGTTGTTTTACAGAAACACACAACATT |
| SEQ ID NO: 1217 | CACGCAAAAAATATTTTAAAACAGGAGCCAAGCAGTGGCTCATGTCCATAATCTCAGTACT |
| | TTGGGAGGCCGAAGCGGGAGGATCACGAGGTCAGGAGAT |
| SEQ ID NO: 1218 | ACTCCCAACAAAAAAGAAATGGCCATTCAGTTGACTCTTGAACAACGAGGGAGGGGCTGG |
| | GGTGCCACCACCTGCACAGTCCAAAATCTGAATTAACTTT |
| SEQ ID NO: 1219 | GGGGGATATTTAAAAAAAAAGATTTCAATCTAGAGGCTACAGGCTGCAGATGCTGGGGGT |
| | GATCACATGAGAGAGCTCTGCTGCAGGGAACGATATGTGA |
| SEQ ID NO: 1220 | CTGTGCCCAGCTCGTGGCCTGTTCTTTGTACTTGCGGGGAAGGGGTCCCCTTCTCCCACAGC |
| | CATCCCCCCGGGGCCTGCTCCCAGGTGGGGTCTGGTTT |
| SEQ ID NO: 1221 | CTGTGCCCAGCTCGTGGCCTGTTCTTTGCACTTGGTGGGGGGGTTCCTATCTCCCACAGCTT |
| | TTCCCCCCGGGGCCTGCTCCCAGGTGGGGTCAGGTTTT |
| SEQ ID NO: 1222 | TTAAGAAAACTAAAAAAAAACAAAACAAACAAAAAAAAAAACTAAAAAAAAAAACCCTA |
| | CATCTTTCTTTCTTTTTTTTTGAGACAGGGTCTCACTCTGT |
| SEQ ID NO: 1223 | AAGGAAAACCTCTTTTTCATGAAATAAAATGAAATAAAACAATTAAAAAATAATAGAGAG |
| | ATCAGAACTTCCCACTAGAGCCATTAGATTCAAAGCTAAT |
| SEQ ID NO: 1224 | CTCAAAACAGAGATGAACTCCCTACTTAAAAATGAGGATATGTGTGCTTTTAATGTCATGC |
| | AGAATCATACAGATTTCCATTTCATTGGACCCTTAGAGG |
| SEQ ID NO: 1225 | TAATGCTATCACTCCCCTTAACCCCCACTCCCTGACAGGCCCCAGTGTGTGATGTTCCCCTT |
| | CCTGTGTCCATGTGTTCTCATTGTTCAACTCCCACTTA |
| SEQ ID NO: 1226 | CATTGAGTTAAAAATGGACGTTTTTTTAAAAAAAGCTAATTAACTAGATTTGCTTACTTAA |
| | AATCAAAGCAATCAACTCGTTACAAGTCTAGAAATAAGG |
| SEQ ID NO: 1227 | CCCCACCAGCCCCCGTCCCCACCCCCCAAAAAAACAGAACCCCACATCACAAACACCCCC |
| | AAGCCCTGGCCCAGGTCTTTCGGATAAAGGGGGCCACTGC |
| SEQ ID NO: 1228 | CAAGTAAATAATAATAAATAAATAAATAAATAAATAAATAAATAAATAATGATTAGATTA |
| | AACTCCAGATCTTTCTGACTCTGAAACCAACTTTCTCCTA |
| SEQ ID NO: 1229 | GAGCCATTCCAGAGATCCAGGAAGCAGGAATCAACGGACTTGCACCTCCAGGTGTTTCAC |
| | CAGCATGAATCTATAGAACTCATATTCCAAGAAGGGTGTT |
| SEQ ID NO: 1230 | TCTCCCCCTAAATCAGGCTATCAAAATTTGCAATGAACAAATAGGACCCAGCTTATATTAC |
| | AAATCTTTGCTGTGATGAGCTGAAACAGGAATATTCTAC |
| SEQ ID NO: 1231 | AAAAAAAAAAAAAAGAAATGGATTATTGAAAGGGAATGGAGATGAAGAAGTTAGAATAG |
| | GTTATGTCAAGGATCCATGAGGTTTGGGTGTTATATCATCA |
| SEQ ID NO: 1232 | CCAATAAGAAAAAAAAATTAAAAGAAAAGAAATCACAAATAAAGTTATAAAATACTCCA |
| | AGATGAATGAAAACAAAAACAAAACATGCCAAAACTTATGG |
| SEQ ID NO: 1233 | TAAAATAAAAACAAAGAAGTCAACGTAATACAAAGCAGAAAAACAATAAAGAAAATCAA |
| | TGAACCCCAAATCTGGTTCTTTCAGAAGAGCGATAAAGTTT |
| SEQ ID NO: 1234 | ACATAAAAAGGAAAAAGAAAAAAAGGAAAAAGTAATAAATTAGTATGAATTGAGCATTT |
| | TAATGATTCTATTTTATTGCCTTTGTTGGCTTATTAAATAT |
| SEQ ID NO: 1235 | AATAAATTTTAAATAAAAAAATAAAAAAAAAGAAACCTGGAACAAAGACTGATTCTGCTC |
| | TATGAAGCCTGTTCTATGAAGGTTCAACCTGCTCACCTGC |
| SEQ ID NO: 1236 | AGGGGAAAAAGAAAAAGGAAATAGACGAGCACATTTGACATTTCTCAGTGGCGTGGCAG |
| | ATCTCTAGGGCTTTATCTCCTTGTCTCATTCAGAGTGTGCC |
| SEQ ID NO: 1237 | CTAAACTAAAAATAGAAATGAAGAAAAAAGAAATGGAAGTTCATAATTTAAAATTTTTAT |
| | TTTTTTGTACAAATTGATGGGGTATAAGTGAAATTTTGTT |
| SEQ ID NO: 1238 | TGAACTTCAAGGATCGATTTAACACTTGTATTTGTGGGCTTGTTACTTATGATAACAGGTG |
| | GCCTAGTTTCATGGCTTTGTGTTTACCGTTTTCGGGTGC |
| SEQ ID NO: 1239 | TTTCTGACGATCACTTACATTTGTGTTATGCTGATTAGCAGATATCCACAAACATAGCTATG |
| | AAGTTCTGACTGGGATAACCTTGCTGTTTGTCTATTTT |
| SEQ ID NO: 1240 | ACCTTATTCACGCCTAAAAAGTAGACTGACTGTGGGGTGGTCGTGTTTTTTGTTTCTTGTTG |
| | GTAGGTGGTGAATGCGTTTTTTTCGTTGTTTTCTCCGT |

In some embodiments, a termination sequence may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289. In some embodiments, the termination sequence comprises a sequence of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289. In some embodiments, the termination sequence is selected from SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289. In some embodiments, a 3′ box sequence element of a termination sequence of any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289 is replaced with a 3′ box sequence element of any of SEQ ID NO: 40-SEQ ID NO: 42 or SEQ ID NO: 121-SEQ ID NO: 166. In some embodiments, a 3′ box sequence element of any of SEQ ID NO: 40-SEQ ID NO: 42 or SEQ ID NO: 121-SEQ ID NO: 166 is inserted or substituted into a termination sequence of any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289. In some embodiments, a 3′ box sequence element from is extracted from any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289 and is inserted into a different termination sequence (e.g., any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289). In some embodiments, the 3′ box sequence element of a termination sequence of any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289 is replaced with a 3′ box sequence element extracted from a different termination sequence (e.g., SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289) or is replaced with a 3′ box sequence element of any of SEQ ID NO: 40-SEQ ID NO: 42 or SEQ ID NO: 121-SEQ ID NO: 166.

Termination sequences of the present disclosure may have insertions or deletions of nucleotides on either side of the termination sequence. Nucleotide bases may be inserted or deleted to the 3′ end of termination sequences to extend the length of the cassette. In some embodiments, a termination sequence of the present disclosure (e.g., any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289) may be truncated by 1 to 2, 1 to 3, 1 to 5, 1 to 10, or 1 to 20 nucleotide bases from the 5′ end, the 3′ end, or both the 5′ end and the 3′ end. In some embodiments, a termination sequence (e.g., any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289) may be truncated by 1, 2, 3, 4, 5, 6, 7, 8, 9, 10, 11, 12, 13, 14, 15, 16, 17, 18, 19, or 20 nucleotides from the 5′ end, the 3′ end, or both the 5′ end and the 3′ end. In some embodiments, 1 to 2, 1 to 3, 1 to 5, 1 to 10, or 1 to 20 nucleotide bases may be added to the 5′ end, the 3′ end, or both the 5′ end and the 3′ end of a termination sequence (e.g., any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289). In some embodiments, 1, 2, 3, 4, 5, 6, 7, 8, 9, 10, 11, 12, 13, 14, 15, 16, 17, 18, 19, or 20 nucleotides may be added to the 5′ end, the 3′ end, or both the 5′ end and the 3′ end of a termination sequence (e.g., any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289). The nucleotides being added to the 5′ end or the 3′ end of the termination sequence may be selected from any nucleotide (e.g., A, T, C, or G). For example, SEQ ID NO: 1254 comprises a 1 nucleotide base deletion on the 5′ end and a 2 nucleotide deletion on the 3′ end of SEQ ID NO: 917. In another example, SEQ ID NO: 1255 comprises a 1 nucleotide base deletion on the 5′ end and a 1 nucleotide base addition to the 3′ end of SEQ ID NO: 709. For example, SEQ ID NO: 1287 comprises a 2 nucleotide base deletion on the 5′ end of SEQ ID NO: 60. For example, SEQ ID NO: 1288 comprises a 4 nucleotide base deletion on the 5′ end of SEQ ID NO: 60. For example, SEQ ID NO: 1289 comprises a 6 nucleotide base deletion on the 5′ end of SEQ ID NO: 60.

A termination sequence (e.g., any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289) may have nucleotide additions on the 3′ end in order to extend the length of the expression cassette. In some embodiments, a termination sequence (e.g., any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289) may have additional nucleotides added to the 3′ end in order to extend the termination sequence to a total length of 100 nucleotides, 150 nucleotides, 200 nucleotides, or 300 nucleotides long. For example, SEQ ID NO: 1264 is an extended version of SEQ ID NO: 1002 with an additional 100 nucleotides added to the 3′ end to extend to a total length of 200 nucleotides. In another example, SEQ ID NO: 1265 is an extended version of SEQ ID NO: 1017 with an additional 100 nucleotides added to the 3′ end to extend to a total length of 200 nucleotides.

Small noncoding RNAs (snRNAs) undergo post-transcriptional cap conversion in which the monomethylguanosine (MMG) cap is converted to a trimethyl guanosine (TMG) cap by the TGSI enzyme. Efficient cap conversion is critical for mature snRNA formation and subsequent transport to the nucleus by snurportin1. A double purine (adenine or guanine) sequence on the 5′ end of a guide RNA may aid in efficient cap conversion. The present disclosure provides for expression cassettes in which the expressed gRNA has an additional 2 bases at the 5′ end, where said additional 2 bases are both purines (adenine or guanine). As such the present disclosure, in some embodiments, provides for expression cassettes having gRNAs that start with an AA, GG, GA, or AG. For example, a SNCA guide RNA (SEQ ID NO: 1290) may have an additional G on the 5′ end resulting in a SNCA guide RNA sequence of SEQ ID NO: 1274 that comprises a GA on the 5′ end.

### Promoter and Termination Sequence Pairings

Expression cassettes of the current disclosure may comprise a promoter sequence (e.g., any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263), a pay load sequence under the transcriptional control of the promoter sequence, and a termination sequence (e.g., any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289).

In some embodiments, the expression cassette comprises a promoter sequence comprising a sequence having at least 80% sequence identity to any one of: a) SEQ ID NO: 17, SEQ ID NO: 1250, or SEQ ID NO: 1262; b) SEQ ID NO: 13 or SEQ ID NO: 15; or c) SEQ ID NO: 1241, SEQ ID NO: 1251, SEQ ID NO: 1252, SEQ ID NO: 1253, or SEQ ID NO: 1263; a payload sequence under transcriptional control of the promoter sequence, the payload sequence comprising a small RNA payload; and a termination sequence comprising a sequence having at least 80% identity to any one of: a) SEQ ID NO: 1002, SEQ ID NO: 1017, SEQ ID NO: 1264, or SEQ ID NO: 1265; or b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269. In some embodiments, the expression cassette comprises a promoter sequence comprising a sequence having at least 80% sequence identity to any one of: a) SEQ ID NO: 17, SEQ ID NO: 1250, or SEQ ID NO: 1262; b) SEQ ID NO: 13 or SEQ ID NO: 15; or c) SEQ ID NO: 1241, SEQ ID NO: 1251, SEQ ID NO: 1252, SEQ ID NO: 1253, or SEQ ID NO: 1263; a payload sequence under transcriptional control of the promoter sequence, the payload sequence comprising a small RNA payload; and a termination sequence. In some embodiments, the expression cassette comprises a promoter sequence; a payload sequence under transcriptional control of the promoter sequence, the payload sequence comprising a small RNA payload; and a termination sequence comprising a sequence having at least 80% identity to any one of: a) SEQ ID NO: 1002, SEQ ID NO: 1017, SEQ ID NO: 1264, or SEQ ID NO: 1265; or b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269.

In some embodiments, the expression cassette comprises a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263; a payload sequence under transcriptional control of the promoter sequence, the payload sequence comprising a small RNA payload; and a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289. In some embodiments, the expression cassette comprises a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263; a payload sequence under transcriptional control of the promoter sequence, the payload sequence comprising a small RNA payload; and a termination sequence. In some embodiments, the expression cassette comprises a promoter sequence; a payload sequence under transcriptional control of the promoter sequence, the payload sequence comprising a small RNA payload; and a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289.

In an embodiment, the expression cassette comprises:
(i) a promotor of SEQ ID NO: 17 and a termination sequence of SEQ ID NO: 1264;
(ii) a promotor of SEQ ID NO: 17 and a termination sequence of SEQ ID NO: 1265;
(iii) a promotor of SEQ ID NO: 17 and a termination sequence of SEQ ID NO: 1254;
(iv) a promotor of SEQ ID NO: 17 and a termination sequence of SEQ ID NO: 1255;
(v) a promotor of SEQ ID NO: 17 and a termination sequence of SEQ ID NO: 1257;
(vi) a promotor of SEQ ID NO: 17 and a termination sequence of SEQ ID NO: 60;
(vii) a promotor of SEQ ID NO: 17 and a termination sequence of SEQ ID NO: 1242;
(viii) a promotor of SEQ ID NO: 1262 and a termination sequence of SEQ ID NO: 1264;
(ix) a promotor of SEQ ID NO: 1262 and a termination sequence of SEQ ID NO: 1265;
(x) a promotor of SEQ ID NO: 1262 and a termination sequence of SEQ ID NO: 1254;
(xi) a promotor of SEQ ID NO: 1262 and a termination sequence of SEQ ID NO: 1255;
(xii) a promotor of SEQ ID NO: 1262 and a termination sequence of SEQ ID NO: 1257;
(xiii) a promotor of SEQ ID NO: 1262 and a termination sequence of SEQ ID NO: 60;
(xiv) a promotor of SEQ ID NO: 1262 and a termination sequence of SEQ ID NO: 1242;
(xv) a promotor of SEQ ID NO: 1250 and a termination sequence of SEQ ID NO: 1264;
(xvi) a promotor of SEQ ID NO: 1250 and a termination sequence of SEQ ID NO: 1265;
(xvii) a promotor of SEQ ID NO: 1250 and a termination sequence of SEQ ID NO: 1254;
(xviii) a promotor of SEQ ID NO: 1250 and a termination sequence of SEQ ID NO: 1255;
(xix) a promotor of SEQ ID NO: 1250 and a termination sequence of SEQ ID NO: 1257;
(xx) a promotor of SEQ ID NO: 1250 and a termination sequence of SEQ ID NO: 60;
(xxi) a promotor of SEQ ID NO: 1250 and a termination sequence of SEQ ID NO: 1242;
(xxii) a promotor of SEQ ID NO: 1251 and a termination sequence of SEQ ID NO: 1264;
(xxiii) a promotor of SEQ ID NO: 1251 and a termination sequence of SEQ ID NO: 1265;
(xxiv) a promotor of SEQ ID NO: 1251 and a termination sequence of SEQ ID NO: 1254;
(xxv) a promotor of SEQ ID NO: 1251 and a termination sequence of SEQ ID NO: 1255;
(xxvi) a promotor of SEQ ID NO: 1251 and a termination sequence of SEQ ID NO: 1257;
(xxvii) a promotor of SEQ ID NO: 1251 and a termination sequence of SEQ ID NO: 60;
(xxviii) a promotor of SEQ ID NO: 1251 and a termination sequence of SEQ ID NO: 1242;
(xxix) a promotor of SEQ ID NO: 1252 and a termination sequence of SEQ ID NO: 1264;
(xxx) a promotor of SEQ ID NO: 1252 and a termination sequence of SEQ ID NO: 1265;
(xxxi) a promotor of SEQ ID NO: 1252 and a termination sequence of SEQ ID NO: 1254;
(xxxii) a promotor of SEQ ID NO: 1252 and a termination sequence of SEQ ID NO: 1255;
(xxxiii) a promotor of SEQ ID NO: 1252 and a termination sequence of SEQ ID NO: 1257;
(xxxiv) a promotor of SEQ ID NO: 1252 and a termination sequence of SEQ ID NO: 60;
(xxxv) a promotor of SEQ ID NO: 1252 and a termination sequence of SEQ ID NO: 1242;
(xxxvi) a promotor of SEQ ID NO: 1253 and a termination sequence of SEQ ID NO: 1264;
(xxxvii) a promotor of SEQ ID NO: 1253 and a termination sequence of SEQ ID NO: 1265;
(xxxviii) a promotor of SEQ ID NO: 1253 and a termination sequence of SEQ ID NO: 1254;
(xxxix) a promotor of SEQ ID NO: 1253 and a termination sequence of SEQ ID NO: 1255;
(xl) a promotor of SEQ ID NO: 1253 and a termination sequence of SEQ ID NO: 1257;
(xli) a promotor of SEQ ID NO: 1253 and a termination sequence of SEQ ID NO: 60;
(xlii) a promotor of SEQ ID NO: 1253 and a termination sequence of SEQ ID NO: 1242;
(xliii) a promotor of SEQ ID NO: 17 and a termination sequence of SEQ ID NO: 1269;
(xliv) a promotor of SEQ ID NO: 1262 and a termination sequence of SEQ ID NO: 1269;
(xlv) a promotor of SEQ ID NO: 1250 and a termination sequence of SEQ ID NO: 1269;
(xlvi) a promotor of SEQ ID NO: 1251 and a termination sequence of SEQ ID NO: 1269;
(xlvii) a promotor of SEQ ID NO: 1252 and a termination sequence of SEQ ID NO: 1269;
(xlviii) a promotor of SEQ ID NO: 1253 and a termination sequence of SEQ ID NO: 1269;
(xlix) a promotor of SEQ ID NO: 17 and a termination sequence of SEQ ID NO: 1017;
(l) a promotor of SEQ ID NO: 1262 and a termination sequence of SEQ ID NO: 1017;
(li) a promotor of SEQ ID NO: 1250 and a termination sequence of SEQ ID NO: 1017;
(lii) a promotor of SEQ ID NO: 1251 and a termination sequence of SEQ ID NO: 1017;
(liii) a promotor of SEQ ID NO: 1252 and a termination sequence of SEQ ID NO: 1017; or
(liv) a promotor of SEQ ID NO: 1253 and a termination sequence of SEQ ID NO: 1017.

### Further Additional Promotor/Termination Sequence Pairings

In an embodiment, the expression cassette comprises a promotor of SEQ ID NO: 17 and a termination sequence of SEQ ID NO: 1264. In an embodiment, the expression cassette comprises a promotor of SEQ ID NO: 1262 and a termination sequence of SEQ ID NO: 1265. In an embodiment, the expression cassette comprises a promotor of SEQ ID NO: 1250 and a termination sequence of SEQ ID NO: 1254. In an embodiment, the expression cassette comprises a promotor of SEQ ID NO: 1251 and a termination sequence of SEQ ID NO: 1255. In an embodiment, the expression cassette comprises a promotor of SEQ ID NO: 1252 and a termination sequence of SEQ ID NO: 1255. In an embodiment, the expression cassette comprises a promotor of SEQ ID NO: 1253 and a termination sequence of SEQ ID NO: 1255. In an embodiment, the expression cassette comprises a promotor of SEQ ID NO: 17 and a termination sequence of SEQ ID NO: 60. In an embodiment, the expression cassette comprises a promotor of SEQ ID NO: 17 and a termination sequence of SEQ ID NO: 1242. In an embodiment, the expression cassette comprises a promotor of SEQ ID NO: 1262 and a termination sequence of SEQ ID NO: 1269. In an embodiment, the expression cassette comprises a promotor of SEQ ID NO: 17 and a termination sequence of SEQ ID NO: 1265. In an embodiment, the expression cassette comprises a promotor of SEQ ID NO: 17 and a termination sequence of SEQ ID NO: 1017.

### Payloads

The expression cassettes of the present disclosure may encode an RNA payload under transcriptional control of a promoter (e.g., an engineered promoter). In some embodiments, the RNA payload may encode a small RNA payload such as a guide sequence (e.g., for RNA or DNA editing), a tracrRNA, an siRNA, an shRNA, or a miRNA, an antisense oligonucleotide (e.g., for expression knockdown), a structural element (e.g., an RNA hairpin), or combinations thereof. Provided herein are engineered RNA payloads and polynucleotides encoding the same; as well as compositions comprising said engineered RNA payloads or said polynucleotides. As used herein, the term “engineered” in reference to an RNA payload or polynucleotide encoding the same refers to a non-naturally occurring RNA or polynucleotide encoding the same. For example, the present disclosure provides for engineered polynucleotides encoding engineered guide RNAs. In some embodiments, the engineered guide comprises RNA. In some embodiments, the engineered guide comprises DNA. In some examples, the engineered guide comprises modified RNA bases or unmodified RNA bases. In some embodiments, the engineered guide comprises modified DNA bases or unmodified DNA bases. In some examples, the engineered guide comprises both DNA and RNA bases.

### Guide RNA Payloads for RNA Editing

The expression cassettes described herein may be used to enhance expression of engineered guide RNAs and engineered polynucleotides encoding the same for site-specific, selective editing of a target RNA via an RNA editing entity or a biologically active fragment thereof. An engineered guide RNA of the present disclosure can comprise latent structures, such that when the engineered guide RNA is hybridized to the target RNA to form a guide-target RNA scaffold, at least a portion of the latent structure manifests as at least a portion of a structural feature as described herein.

An engineered guide RNA, as described herein, may comprise a targeting domain with complementarity to a target RNA described herein. As such, a guide RNA can be engineered to site-specifically/selectively target and hybridize to a particular target RNA, thus facilitating editing of specific nucleotide in the target RNA via an RNA editing entity or a biologically active fragment thereof. The targeting domain can include a nucleotide that is positioned such that, when the guide RNA is hybridized to the target RNA, the nucleotide opposes a base to be edited by the RNA editing entity or biologically active fragment thereof and does not base pair, or does not fully base pair, with the base to be edited. This mismatch can help to localize editing of the RNA editing entity to the desired base of the target RNA. However, in some instances there can be some, and in some cases significant, off target editing in addition to the desired edit.

Hybridization of the target RNA and the targeting domain of the guide RNA may produce specific secondary structures in the guide-target RNA scaffold that manifest upon hybridization, which are referred to herein as “latent structures.” Latent structures, when manifested, may become structural features described herein, including mismatches, bulges, internal loops, and hairpins. Without wishing to be bound by theory, the presence of structural features described herein that are produced upon hybridization of the guide RNA with the target RNA configure the guide RNA to facilitate a specific, or selective, targeted edit of the target RNA via the RNA editing entity or biologically active fragment thereof. Further, the structural features in combination with the mismatch described above generally facilitate an increased amount of editing of a target residue (e.g., an adenosine residue), fewer off target edits, or both, as compared to a construct comprising the mismatch alone or a construct having perfect complementarity to a target RNA. Accordingly, rational design of latent structures in engineered guide RNAs of the present disclosure to produce specific structural features in a guide-target RNA scaffold can be a powerful tool to promote editing of the target RNA with high specificity, selectivity, and robust activity.

In some examples, the engineered guides provided herein comprise an engineered guide that can be configured, upon hybridization to a target RNA molecule, to form, at least in part, a guide-target RNA scaffold with at least a portion of the target RNA molecule, wherein the guide-target RNA scaffold comprises at least one structural feature, and wherein the guide-target RNA scaffold recruits an RNA editing entity and facilitates a chemical modification of a base of a nucleotide in the target RNA molecule by the RNA editing entity.

In some examples, a target RNA of an engineered guide RNA of the present disclosure can be a pre-mRNA or mRNA. In some embodiments, the engineered guide RNA of the present disclosure hybridizes to a sequence of the target RNA. In some embodiments, part of the engineered guide RNA (e.g., a targeting domain) hybridizes to the sequence of the target RNA. The part of the engineered guide RNA that hybridizes to the target RNA is of sufficient complementary to the sequence of the target RNA for hybridization to occur.

Targeting Domain. Engineered guide RNAs disclosed herein can be engineered in any way suitable for RNA editing. In some examples, an engineered guide RNA generally comprises at least a targeting sequence that allows it to hybridize to a region of a target RNA molecule. A targeting sequence can also be referred to as a “targeting domain” or a “targeting region.”

As used herein, the term “targeting sequence” can be used interchangeable with “targeting domain” or “targeting region” and refers to a polynucleotide sequence within an engineered guide RNA sequence that is at least partially complementary to a target polynucleotide. The target polynucleotide (e.g., a target RNA or a target DNA) may be a region of a polynucleotide of interest, such as a gene or a messenger RNA. As used herein, a “complementary” sequence refers to a sequence that is a reverse complement relative to a second sequence.

A targeting sequence of an engineered guide RNA allows the engineered guide RNA to hybridize to a target polynucleotide (e.g., a target RNA) through base pairing, such as Watson Crick base pairing. A targeting sequence can be located at either the N-terminus or C-terminus of the engineered guide RNA, or both, or the targeting sequence can be within the engineered guide RNA. The targeting sequence can be of any length sufficient to hybridize with the target polynucleotide. In some cases, the targeting sequence is at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or up to about 200 nucleotides in length. In an embodiment, an engineered polynucleotide comprises a targeting sequence that is about 25 to 200, 50 to 150, 75 to 100, 80 to 110, 90 to 120, 95 to 115, 60 to 200, 60 to 180, 60 to 160, 60 to 140, 70 to 200, 70 to 180, 70 to 160, 70 to 140, 80 to 200, 80 to 190, 80 to 170, 80 to 160, 80 to 150, 80 to 140, 80 to 130, 80 to 120, 90 to 200, 90 to 190, 90 to 180, 90 to 170, 90 to 160, 90 to 150, 90 to 140, 90 to 130, 90 to 120, 100 to 200, 100 to 190, 100 to 180, 100 to 170, 100 to 160, 100 to 150, 100 to 140, 100 to 130, 100 to 120, 110 to 200, 110 to 190, 110 to 180, 110 to 170, 110 to 160, 110 to 150, 110 to 140, 110 to 120, 120 to 200, 120 to 190, 120 to 180, 120 to 170, 120 to 160, 120 to 150, 120 to 140, 130 to 200, 130 to 190, 130 to 180, 130 to 170, 130 to 160, 130 to 150, 140 to 200, 140 to 190, 140 to 180, 140 to 170, 140 to 160, 150 to 200, 150 to 190, 150 to 180, 150 to 170, 160 to 200, 160 to 190 or 160 to 180 nucleotides in length.

A targeting sequence comprises at least partial sequence complementarity to a target polynucleotide. The targeting sequence may have a degree of sequence complementarity to the target polynucleotide sufficient to hybridize with the target polynucleotide. In some cases, the targeting sequence comprises 95%, 96%, 97%, 98%, 99%, or 100% sequence complementarity to the target polynucleotide. In some cases, the targeting sequence comprises less than 100% complementarity to the target polynucleotide sequence. For example, the targeting sequence may have a single base mismatch relative to the target polynucleotide when bound to the target polynucleotide. In other cases, the targeting sequence comprises at least about 1, 2, 3, 4, 5, 6, 7, 8, 9, 10, 11, 12, 13, 14, 15, 16, 20, 30, 40 or up to about 50 base mismatches relative to the target polynucleotide when bound to the target polynucleotide. In some aspects, nucleotide mismatches can be associated with structural features provided herein. In some aspects, a targeting sequence comprises at least about 1, 2, 3, 4, 5, 6, 7, 8, 9, 10, 11, 12, 13, 14, or up to about 15 nucleotides that differ in complementarity from a wildtype polynucleotide of a subject target polynucleotide.

A targeting sequence comprises nucleotide residues having complementarity to a target polynucleotide. The targeting sequence may have a number of residues with complementarity to the target polynucleotide sufficient to hybridize with the target polynucleotide. The complementary residues may be contiguous or non-contiguous. In some cases, the targeting sequence comprises at least 50 nucleotides having complementarity to the target polynucleotide. In some cases, the targeting sequence comprises from 50 to 150 nucleotides having complementarity to the target polynucleotide. In some cases, the targeting sequence comprises from 50 to 200 nucleotides having complementarity to the target polynucleotide. In some cases, the targeting sequence comprises from 50 to 250 nucleotides having complementarity to the target polynucleotide. In some cases, the targeting sequence comprises from 50 to 300 nucleotides having complementarity to the target polynucleotide. In some cases, the targeting sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 nucleotides having complementarity to the target polynucleotide. In some cases, the targeting sequence comprises more than 50 nucleotides total and has at least 50 nucleotides having complementarity to the target polynucleotide. In some cases, the targeting sequence comprises from 50 to 400 nucleotides total and has from 50 to 150 nucleotides having complementarity to the target polynucleotide. In some cases, the targeting sequence comprises from 50 to 400 nucleotides total and has from 50 to 200 nucleotides having complementarity to the target polynucleotide. In some cases, the targeting sequence comprises from 50 to 400 nucleotides total and has from 50 to 250 nucleotides having complementarity to the target polynucleotide. In some cases, the targeting sequence comprises from 50 to 400 nucleotides total and has from 50 to 300 nucleotides having complementarity to the target polynucleotide. In some cases, the at least 50 nucleotides having complementarity to the target polynucleotide are separated by one or more mismatches, one or more bulges, or one or more loops, or any combination thereof. In some cases, the from 50 to 150 nucleotides having complementarity to the target polynucleotide are separated by one or more mismatches, one or more bulges, or one or more loops, or any combination thereof. In some cases, the from 50 to 200 nucleotides having complementarity to the target polynucleotide are separated by one or more mismatches, one or more bulges, or one or more loops, or any combination thereof. In some cases, the from 50 to 250 nucleotides having complementarity to the target polynucleotide are separated by one or more mismatches, one or more bulges, or one or more loops, or any combination thereof. In some cases, the from 50 to 300 nucleotides having complementarity to the target polynucleotide are separated by one or more mismatches, one or more bulges, or one or more loops, or any combination thereof. For example, a targeting sequence comprises a total of 54 nucleotides wherein, sequentially, 25 nucleotides are complementarity to the target polynucleotide, 4 nucleotides form a bulge, and 25 nucleotides are complementarity to the target polynucleotide. As another example, a targeting sequence comprises a total of 118 nucleotides wherein, sequentially, 25 nucleotides are complementarity to the target polynucleotide, 4 nucleotides form a bulge, 25 nucleotides are complementarity to the target polynucleotide, 14 nucleotides form a loop, and 50 nucleotides are complementary to the target polynucleotide.

In some cases, a targeting domain comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence can have a single base mismatch.

The targeting sequence can have sufficient complementarity to a target RNA to allow for hybridization of the targeting sequence to the target RNA. In some embodiments, the targeting sequence has a minimum antisense complementarity of about 50 nucleotides or more to the target RNA. In some embodiments, the targeting sequence has a minimum antisense complementarity of about 60 nucleotides or more to the target RNA. In some embodiments, the targeting sequence has a minimum antisense complementarity of about 70 nucleotides or more to the target RNA. In some embodiments, the targeting sequence has a minimum antisense complementarity of about 80 nucleotides or more to the target RNA. In some embodiments, the targeting sequence has a minimum antisense complementarity of about 90 nucleotides or more to the target RNA. In some embodiments, the targeting sequence has a minimum antisense complementarity of about 100 nucleotides or more to the target RNA. In some embodiments, antisense complementarity refers to non-contiguous stretches of sequence. In some embodiments, antisense complementarity refers to contiguous stretches of sequence.

In some embodiments, hybridization of the targeting sequence to the target RNA to form a guide-target RNA scaffold may manifest a latent structural feature. For example, a latent structural feature may comprise a symmetric bulge, an asymmetric bulge, a symmetric internal loop, an asymmetric internal loop, or combinations thereof. In some embodiments, the latent structural feature may comprise 1, 2, 3, 4, 5, 6, 7, 8, 9, 10, 11, 12, 13, 14, 15, 16, 17, 18, 19, or 20 unpaired nucleotides on the target RNA side. In some embodiments, the latent structural feature may comprise 1, 2, 3, 4, 5, 6, 7, 8, 9, 10, 11, 12, 13, 14, 15, 16, 17, 18, 19, or 20 unpaired nucleotides on the guide RNA side.

In some embodiments an engineered guide RNA for RNA editing may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to SEQ ID NO: 1273, SEQ ID NO: 1274, SEQ ID NO: 61, or SEQ ID NO: 1290. For example, an engineered guide RNA of SEQ ID NO: 1273 may be used to target PMP22. In another example, an engineered guide RNA of SEQ ID NO: 1274 may be used to target SNCA. In another example, an engineered guide RNA of SEQ ID NO: 1290 may be used to target SNCA. In another example, an engineered guide RNA of SEQ ID NO: 61 may be used to target SERPINA1. Examples of engineered guide RNAs are provided in TABLE 8.

TABLE 8
Engineered Guide RNAs

| | SEQ ID | |
|---|---|---|
| Target | NO: | Sequence |
| PMP22 | SEQ ID | GACCGCACCAGCACCGCGACGTGGAGGACGATG |
| | NO: | ATACTCAGCAACAGGAGGAGCCCACTGGCGGCA |
| | 1273 | AGTTCTGCTCAGCGGAGTTTCTGCCCGGCCAAA |
| | | CAGCGTGTGGAATTTTTGGAGCAGGTTTTCTGA |
| | | CTTCGGTCGGAAAACCCCT |
| SNCA | SEQ ID | GACCGGCCACAACTCCCTCCTTGGCCTTTGAAA |
| | NO: | GTCCTTTCATGAATACATCCACGGCTAATGAAT |
| | 1274 | TCCTTTACACCACACTGGAAAACATAAAATACA |
| | | CTTTGAGTGGAATTTTTGGAGCAGGTTTTCTGA |
| | | CTTCGGTCGGAAAACCCCT |
| SNCA | SEQ ID | ACCGGCCACAACTCCCTCCTTGGCCTTTGAAAG |
| | NO: | TCCTTTCATGAATACATCCACGGCTAATGAATT |
| | 1290 | CCTTTACACCACACTGGAAAACATAAAATACAC |
| | | TTTGAGTGGAATTTTTGGAGCAGGTTTTCTGAC |
| | | TTCGGTCGGAAAACCCCT |
| SERPINA1 | SEQ ID | GACCGTAGACATGGGTATGGCCTCTAATTTGTA |
| | NO: 61 | GGCCCCAGCAGCTTCAGTCCCTTACTCGTCGTA |
| | | CCAGAGCACAGCCAGTCGTATGCACGGCGTGGA |
| | | ATTTTTGGAGCAGGTTTTCTGACTTCGGTCGGA |
| | | AAACCCCT |

Engineered Guide RNAs Having a Recruitment Domain. In some examples, a subject engineered guide RNA comprises a recruiting domain that recruits an RNA editing entity (e.g., ADAR), where in some instances, the recruiting domain is formed and present in the absence of binding to the target RNA. A “recruiting domain” can be referred to herein as a “recruiting sequence” or a “recruiting region”. In some examples, a subject engineered guide can facilitate editing of a base of a nucleotide of in a target sequence of a target RNA that results in modulating the expression of a polypeptide encoded by the target RNA. In some instances, modulation can be increased or decrease expression of the polypeptide. In some cases, an engineered guide can be configured to facilitate an editing of a base of a nucleotide or polynucleotide of a region of an RNA by an RNA editing entity (e.g., ADAR or APOBEC). In order to facilitate editing, an engineered polynucleotide of the disclosure can recruit an RNA editing entity (e.g., ADAR or APOBEC). Various RNA editing entity recruiting domains can be utilized. In some examples, a recruiting domain comprises: Glutamate ionotropic receptor AMPA type subunit 2 (GluR2), an Alu sequence, or, in the case of recruiting APOBEC, an APOBEC recruiting domain.

In some examples, more than one recruiting domain can be included in an engineered guide of the disclosure. In examples where a recruiting domain can be present, the recruiting domain can be utilized to position the RNA editing entity to effectively react with a subject target RNA after the targeting sequence hybridizes to a target sequence of a target RNA. In some cases, a recruiting domain can allow for transient binding of the RNA editing entity to the engineered guide. In some examples, the recruiting domain allows for permanent binding of the RNA editing entity to the engineered guide. A recruiting domain can be of any length. In some cases, a recruiting domain can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting domain can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting domain can be about 45 nucleotides in length. In some cases, at least a portion of a recruiting domain comprises at least 1 to about 75 nucleotides. In some cases, at least a portion of a recruiting domain comprises about 45 nucleotides to about 60 nucleotides.

In some embodiments, a recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 70%, 80%, 85%, 90%, 95%, 98%, 99%, or 100% identity and/or length to: GUGGAAUAGUAUAACAAUAUGCUAAAUGUUGUUAUAGUAUCCCAC (SEQ ID NO: 51). In some cases, a recruiting domain can comprise at least about 80% sequence homology to at least about 10, 15, 20, 25, or 30 nucleotides of SEQ ID NO: 51. In some examples, a recruiting domain can comprise at least about 90%, 95%, 96%, 97%, 98%, or 99% sequence homology and/or length to SEQ ID NO: 51.

Additional, RNA editing entity recruiting domains are also contemplated. In an embodiment, a recruiting domain comprises an apolipoprotein B mRNA editing enzyme, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In another embodiment, a recruiting domain can be from an Alu domain.

Any number of recruiting domains can be found in an engineered guide of the present disclosure. In some examples, at least about 1, 2, 3, 4, 5, 6, 7, 8, 9, or up to about 10 recruiting domains can be included in an engineered guide. Recruiting domains can be located at any position of engineered guide RNAs. In some cases, a recruiting domain can be on an N-terminus, middle, or C-terminus of an engineered guide RNA. A recruiting domain can be upstream or downstream of a targeting sequence. In some cases, a recruiting domain flanks a targeting sequence of a subject guide. A recruiting sequence can comprise all ribonucleotides or deoxyribonucleotides, although a recruiting domain comprising both ribo- and deoxyribonucleotides can in some cases not be excluded.

Engineered Guide RNAs with Latent Structure. In some examples, an engineered guide disclosed herein useful for facilitating editing of a target RNA by an RNA editing entity can be an engineered latent guide RNA. An “engineered latent guide RNA” refers to an engineered guide RNA that comprises latent structure. “Latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed, and the latent structure provided in the guide RNA is, thus, unmasked. The formation and structure of a latent structural feature upon binding to the target RNA depends on the guide RNA sequence. For example, formation and structure of the latent structural feature may depend on a pattern of complementary and mismatched residues in the guide RNA sequence relative to the target RNA. The guide RNA sequence may be engineered to have a latent structural feature that forms upon binding to the target RNA.

A double stranded RNA (dsRNA) substrate may be formed upon hybridization of an engineered guide RNA of the present disclosure to a target RNA. The resulting dsRNA substrate is also referred to herein as a “guide-target RNA scaffold.”

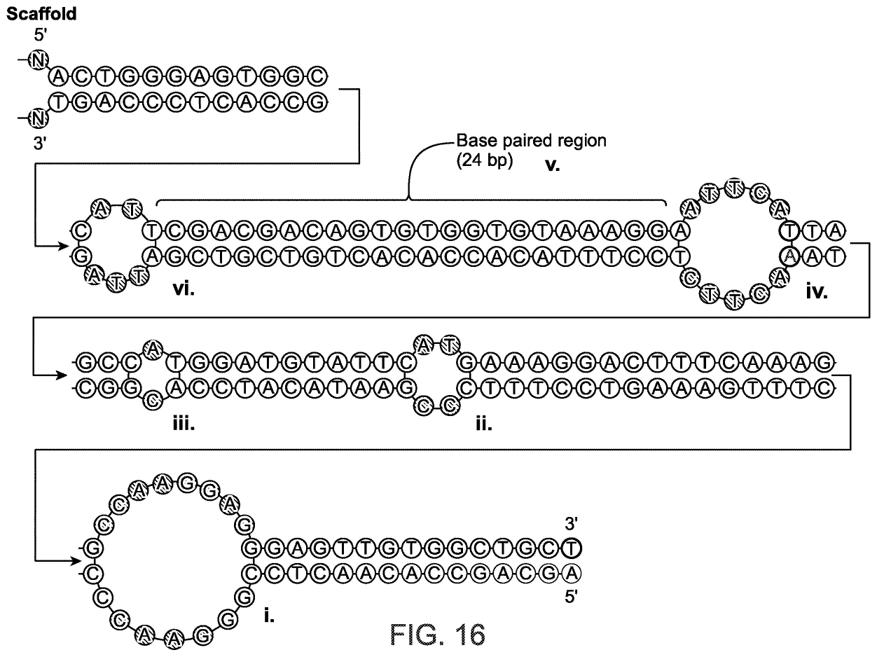

FIG. 16 shows a legend of various exemplary structural features present in guide-target RNA scaffolds formed upon hybridization of a latent guide RNA of the present disclosure to a target RNA. Example structural features shown include an 8/7 asymmetric loop (i., 8 nucleotides on the target RNA side and 7 nucleotides on the guide RNA side), a 2/2 symmetric bulge (ii., 2 nucleotides on the target RNA side and 2 nucleotides on the guide RNA side), a 1/1 mismatch (iii., 1 nucleotide on the target RNA side and 1 nucleotide on the guide RNA side), a 5/5 symmetric internal loop (iv., 5 nucleotides on the target RNA side and 5 nucleotides on the guide RNA side), a 24 bp region (v., 24 nucleotides on the target RNA side base paired to 24 nucleotides on the guide RNA side), and a 2/3 asymmetric bulge (vi., 2 nucleotides on the target RNA side and 3 nucleotides on the guide RNA side).

Unless otherwise noted, the number of participating nucleotides in a given structural feature is indicated as the nucleotides on the target RNA side over nucleotides on the guide RNA side. Also shown in this legend is a key to the positional annotation of each figure. For example, the target nucleotide to be edited is designated as the 0 position. Downstream (3′) of the target nucleotide to be edited, each nucleotide is counted in increments of +1. Upstream (5′) of the target nucleotide to be edited, each nucleotide is counted in increments of −1. Thus, the example 2/2 symmetric bulge in this legend is at the +12 to +13 position in the guide-target RNA scaffold. Similarly, the 2/3 asymmetric bulge in this legend is at the −36 to −37 position in the guide-target RNA scaffold. As used herein, positional annotation is provided with respect to the target nucleotide to be edited and on the target RNA side of the guide-target RNA scaffold. As used herein, if a single position is annotated, the structural feature extends from that position away from position 0 (target nucleotide to be edited). For example, if a latent guide RNA is annotated herein as forming a 2/3 asymmetric bulge at position −36, then the 2/3 asymmetric bulge forms from −36 position to the −37 position with respect to the target nucleotide to be edited (position 0) on the target RNA side of the guide-target RNA scaffold. As another example, if a latent guide RNA is annotated herein as forming a 2/2 symmetric bulge at position +12, then the 2/2 symmetric bulge forms from the +12 to the +13 position with respect to the target nucleotide to be edited (position 0) on the target RNA side of the guide-target RNA scaffold.

In some examples, the engineered guides disclosed herein lack a recruiting region and recruitment of the RNA editing entity can be effectuated by structural features of the guide-target RNA scaffold formed by hybridization of the engineered guide RNA and the target RNA. In some examples, the engineered guide, when present in an aqueous solution and not bound to the target RNA molecule, does not comprise structural features that recruit the RNA editing entity (e.g., ADAR or APOBEC). The engineered guide RNA, upon hybridization to a target RNA, form with the target RNA molecule, one or more structural features that recruits an RNA editing entity (e.g., ADAR or APOBEC).

In cases where a recruiting sequence can be absent, an engineered guide RNA can be still capable of associating with a subject RNA editing entity (e.g., ADAR or APOBEC) to facilitate editing of a target RNA and/or modulate expression of a polypeptide encoded by a subject target RNA. This can be achieved through structural features formed in the guide-target RNA scaffold formed upon hybridization of the engineered guide RNA and the target RNA. Structural features can comprise any one of a: mismatch, symmetrical bulge, asymmetrical bulge, symmetrical internal loop, asymmetrical internal loop, hairpins, wobble base pairs, or any combination thereof.

Described herein are structural features which can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

A guide-target RNA scaffold may be formed upon hybridization of an engineered guide RNA of the present disclosure to a target RNA. As disclosed herein, a mismatch refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA.

In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA.

In another aspect, a structural feature comprises a wobble base. A wobble base pair refers to two bases that weakly base pair. For example, a wobble base pair of the present disclosure can refer to a G paired with a U. Thus, a wobble base pair can be a structural feature formed from latent structure provided by an engineered latent guide RNA.

In some cases, a structural feature can be a hairpin. As disclosed herein, a hairpin includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can include a recruitment hairpin or a non-recruitment hairpin. A hairpin can be located anywhere within the engineered guide RNAs of the present disclosure. In some embodiments, one or more hairpins is proximal to or present at the 3′ end of an engineered guide RNA of the present disclosure, proximal to or at the 5′ end of an engineered guide RNA of the present disclosure, proximal to or within the targeting domain of the engineered guide RNAs of the present disclosure, or any combination thereof.

In some aspects, a structural feature comprises a non-recruitment hairpin. A non-recruitment hairpin, as disclosed herein, does not have a primary function of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in vitro assay. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by latent structure provided in an engineered latent guide RNA.

A hairpin of the present disclosure can be of any length. In an aspect, a hairpin can be from about 10-500 or more nucleotides. In some cases, a hairpin can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides. In other cases, a hairpin can also comprise 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

A guide-target RNA scaffold is formed upon hybridization of an engineered guide RNA of the present disclosure to a target RNA. As disclosed herein, a bulge refers to the structure substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

A guide-target RNA scaffold is formed upon hybridization of an engineered guide RNA of the present disclosure to a target RNA. A bulge can be a symmetrical bulge or an asymmetrical bulge. A symmetrical bulge is formed when the same number of nucleotides is present on each side of the bulge. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold target and 2 nucleotides on the target RNA side of the guide-target RNA scaffold. A symmetrical bulge of the present disclosure can be formed by 3 nucleotides on the engineered guide RNA side of the guide-target RNA scaffold target and 3 nucleotides on the target RNA side of the guide-target RNA scaffold. A symmetrical bulge of the present disclosure can be formed by 4 nucleotides on the engineered guide RNA side of the guide-target RNA scaffold target and 4 nucleotides on the target RNA side of the guide-target RNA scaffold. Thus, a symmetrical bulge can be a structural feature formed from latent structure provided by an engineered latent guide RNA.

A guide-target RNA scaffold is formed upon hybridization of an engineered guide RNA of the present disclosure to a target RNA. A bulge can be a symmetrical bulge or an asymmetrical bulge. An asymmetrical bulge is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. An asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

In some cases, a structural feature can be an internal loop. As disclosed herein, an internal loop refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can help with base flipping of the target A in the target RNA to be edited.

One side of the internal loop, either on the target RNA side or the engineered guide RNA side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1000 nucleotides. Thus, an internal loop can be a structural feature formed from latent structure provided by an engineered latent guide RNA.

An internal loop can be a symmetrical internal loop or an asymmetrical internal loop. A symmetrical internal loop is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 15 nucleotides on the engineered guide RNA side of the guide-target RNA scaffold target and 15 nucleotides on the target RNA side of the guide-target RNA scaffold. A symmetrical internal loop of the present disclosure can be formed by 20 nucleotides on the engineered guide RNA side of the guide-target RNA scaffold target and 20 nucleotides on the target RNA side of the guide-target RNA scaffold. A symmetrical internal loop of the present disclosure can be formed by 30 nucleotides on the engineered guide RNA side of the guide-target RNA scaffold target and 30 nucleotides on the target RNA side of the guide-target RNA scaffold. A symmetrical internal loop of the present disclosure can be formed by 40 nucleotides on the engineered guide RNA side of the guide-target RNA scaffold target and 40 nucleotides on the target RNA side of the guide-target RNA scaffold. A symmetrical internal loop of the present disclosure can be formed by 50 nucleotides on the engineered guide RNA side of the guide-target RNA scaffold target and 50 nucleotides on the target RNA side of the guide-target RNA scaffold. A symmetrical internal loop of the present disclosure can be formed by 60 nucleotides on the engineered guide RNA side of the guide-target RNA scaffold target and 60 nucleotides on the target RNA side of the guide-target RNA scaffold. A symmetrical internal loop of the present disclosure can be formed by 70 nucleotides on the engineered guide RNA side of the guide-target RNA scaffold target and 70 nucleotides on the target RNA side of the guide-target RNA scaffold. A symmetrical internal loop of the present disclosure can be formed by 80 nucleotides on the engineered guide RNA side of the guide-target RNA scaffold target and 80 nucleotides on the target RNA side of the guide-target RNA scaffold. A symmetrical internal loop of the present disclosure can be formed by 90 nucleotides on the engineered guide RNA side of the guide-target RNA scaffold target and 90 nucleotides on the target RNA side of the guide-target RNA scaffold. A symmetrical internal loop of the present disclosure can be formed by 100 nucleotides on the engineered guide RNA side of the guide-target RNA scaffold target and 100 nucleotides on the target RNA side of the guide-target RNA scaffold. A symmetrical internal loop of the present disclosure can be formed by 110 nucleotides on the engineered guide RNA side of the guide-target RNA scaffold target and 110 nucleotides on the target RNA side of the guide-target RNA scaffold. A symmetrical internal loop of the present disclosure can be formed by 120 nucleotides on the engineered guide RNA side of the guide-target RNA scaffold target and 120 nucleotides on the target RNA side of the guide-target RNA scaffold. A symmetrical internal loop of the present disclosure can be formed by 130 nucleotides on the engineered guide RNA side of the guide-target RNA scaffold target and 130 nucleotides on the target RNA side of the guide-target RNA scaffold. A symmetrical internal loop of the present disclosure can be formed by 140 nucleotides on the engineered guide RNA side of the guide-target RNA scaffold target and 140 nucleotides on the target RNA side of the guide-target RNA scaffold. A symmetrical internal loop of the present disclosure can be formed by 150 nucleotides on the engineered guide RNA side of the guide-target RNA scaffold target and 150 nucleotides on the target RNA side of the guide-target RNA scaffold. A symmetrical internal loop of the present disclosure can be formed by 200 nucleotides on the engineered guide RNA side of the guide-target RNA scaffold target and 200 nucleotides on the target RNA side of the guide-target RNA scaffold. A symmetrical internal loop of the present disclosure can be formed by 250 nucleotides on the engineered guide RNA side of the guide-target RNA scaffold target and 250 nucleotides on the target RNA side of the guide-target RNA scaffold. A symmetrical internal loop of the present disclosure can be formed by 300 nucleotides on the engineered guide RNA side of the guide-target RNA scaffold target and 300 nucleotides on the target RNA side of the guide-target RNA scaffold. A symmetrical internal loop of the present disclosure can be formed by 350 nucleotides on the engineered guide RNA side of the guide-target RNA scaffold target and 350 nucleotides on the target RNA side of the guide-target RNA scaffold. A symmetrical internal loop of the present disclosure can be formed by 400 nucleotides on the engineered guide RNA side of the guide-target RNA scaffold target and 400 nucleotides on the target RNA side of the guide-target RNA scaffold. A symmetrical internal loop of the present disclosure can be formed by 450 nucleotides on the engineered guide RNA side of the guide-target RNA scaffold target and 450 nucleotides on the target RNA side of the guide-target RNA scaffold. A symmetrical internal loop of the present disclosure can be formed by 500 nucleotides on the engineered guide RNA side of the guide-target RNA scaffold target and 500 nucleotides on the target RNA side of the guide-target RNA scaffold. A symmetrical internal loop of the present disclosure can be formed by 600 nucleotides on the engineered guide RNA side of the guide-target RNA scaffold target and 600 nucleotides on the target RNA side of the guide-target RNA scaffold. A symmetrical internal loop of the present disclosure can be formed by 700 nucleotides on the engineered guide RNA side of the guide-target RNA scaffold target and 700 nucleotides on the target RNA side of the guide-target RNA scaffold. A symmetrical internal loop of the present disclosure can be formed by 800 nucleotides on the engineered guide RNA side of the guide-target RNA scaffold target and 800 nucleotides on the target RNA side of the guide-target RNA scaffold. A symmetrical internal loop of the present disclosure can be formed by 900 nucleotides on the engineered guide RNA side of the guide-target RNA scaffold target and 900 nucleotides on the target RNA side of the guide-target RNA scaffold. A symmetrical internal loop of the present disclosure can be formed by 1000 nucleotides on the engineered guide RNA side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

An asymmetrical internal loop is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered guide RNA side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered guide RNA side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered guide RNA side of the guide-target RNA scaffold. An asymmetrical internal loop of the present disclosure can be formed by 5 nucleotides on the target RNA side of the guide-target RNA scaffold and 100 nucleotides on the engineered guide RNA side of the guide-target RNA scaffold. An asymmetrical internal loop of the present disclosure can be formed by 5 nucleotides on the target RNA side of the guide-target RNA scaffold and 150 nucleotides on the engineered guide RNA side of the guide-target RNA scaffold. An asymmetrical internal loop of the present disclosure can be formed by 5 nucleotides on the target RNA side of the guide-target RNA scaffold and 200 nucleotides on the engineered guide RNA side of the guide-target RNA scaffold. An asymmetrical internal loop of the present disclosure can be formed by 5 nucleotides on the target RNA side of the guide-target RNA scaffold and 300 nucleotides on the engineered guide RNA side of the guide-target RNA scaffold. An asymmetrical internal loop of the present disclosure can be formed by 5 nucleotides on the target RNA side of the guide-target RNA scaffold and 400 nucleotides on the engineered guide RNA side of the guide-target RNA scaffold. An asymmetrical internal loop of the present disclosure can be formed by 5 nucleotides on the target RNA side of the guide-target RNA scaffold and 500 nucleotides on the engineered guide RNA side of the guide-target RNA scaffold. An asymmetrical internal loop of the present disclosure can be formed by 5 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 5 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 5 nucleotides on the engineered guide RNA side of the guide-target RNA scaffold. An asymmetrical internal loop of the present disclosure can be formed by 300 nucleotides on the target RNA side of the guide-target RNA scaffold and 5 nucleotides on the engineered guide RNA side of the guide-target RNA scaffold. An asymmetrical internal loop of the present disclosure can be formed by 200 nucleotides on the target RNA side of the guide-target RNA scaffold and 5 nucleotides on the engineered guide RNA side of the guide-target RNA scaffold. An asymmetrical internal loop of the present disclosure can be formed by 150 nucleotides on the target RNA side of the guide-target RNA scaffold and 5 nucleotides on the engineered guide RNA side of the guide-target RNA scaffold. An asymmetrical internal loop of the present disclosure can be formed by 100 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 100 nucleotides on the engineered guide RNA side of the guide-target RNA scaffold. An asymmetrical internal loop of the present disclosure can be formed by 50 nucleotides on the target RNA side of the guide-target RNA scaffold and 150 nucleotides on the engineered guide RNA side of the guide-target RNA scaffold. An asymmetrical internal loop of the present disclosure can be formed by 50 nucleotides on the target RNA side of the guide-target RNA scaffold and 200 nucleotides on the engineered guide RNA side of the guide-target RNA scaffold. An asymmetrical internal loop of the present disclosure can be formed by 50 nucleotides on the target RNA side of the guide-target RNA scaffold and 300 nucleotides on the engineered guide RNA side of the guide-target RNA scaffold. An asymmetrical internal loop of the present disclosure can be formed by 50 nucleotides on the target RNA side of the guide-target RNA scaffold and 400 nucleotides on the engineered guide RNA side of the guide-target RNA scaffold. An asymmetrical internal loop of the present disclosure can be formed by 50 nucleotides on the target RNA side of the guide-target RNA scaffold and 500 nucleotides on the engineered guide RNA side of the guide-target RNA scaffold. An asymmetrical internal loop of the present disclosure can be formed by 50 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 50 nucleotides on the engineered guide RNA side of the guide-target RNA scaffold. An asymmetrical internal loop of the present disclosure can be formed by 500 nucleotides on the target RNA side of the guide-target RNA scaffold and 50 nucleotides on the engineered guide RNA side of the guide-target RNA scaffold. An asymmetrical internal loop of the present disclosure can be formed by 400 nucleotides on the target RNA side of the guide-target RNA scaffold and 50 nucleotides on the engineered guide RNA side of the guide-target RNA scaffold. An asymmetrical internal loop of the present disclosure can be formed by 300 nucleotides on the target RNA side of the guide-target RNA scaffold and 50 nucleotides on the engineered guide RNA side of the guide-target RNA scaffold. An asymmetrical internal loop of the present disclosure can be formed by 200 nucleotides on the target RNA side of the guide-target RNA scaffold and 50 nucleotides on the engineered guide RNA side of the guide-target RNA scaffold. An asymmetrical internal loop of the present disclosure can be formed by 150 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 150 nucleotides on the engineered guide RNA side of the guide-target RNA scaffold. An asymmetrical internal loop of the present disclosure can be formed by 100 nucleotides on the target RNA side of the guide-target RNA scaffold and 200 nucleotides on the engineered guide RNA side of the guide-target RNA scaffold. An asymmetrical internal loop of the present disclosure can be formed by 100 nucleotides on the target RNA side of the guide-target RNA scaffold and 300 nucleotides on the engineered guide RNA side of the guide-target RNA scaffold. An asymmetrical internal loop of the present disclosure can be formed by 100 nucleotides on the target RNA side of the guide-target RNA scaffold and 400 nucleotides on the engineered guide RNA side of the guide-target RNA scaffold. An asymmetrical internal loop of the present disclosure can be formed by 100 nucleotides on the target RNA side of the guide-target RNA scaffold and 500 nucleotides on the engineered guide RNA side of the guide-target RNA scaffold. An asymmetrical internal loop of the present disclosure can be formed by 100 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 100 nucleotides on the engineered guide RNA side of the guide-target RNA scaffold. An asymmetrical internal loop of the present disclosure can be formed by 500 nucleotides on the target RNA side of the guide-target RNA scaffold and 100 nucleotides on the engineered guide RNA side of the guide-target RNA scaffold. An asymmetrical internal loop of the present disclosure can be formed by 400 nucleotides on the target RNA side of the guide-target RNA scaffold and 100 nucleotides on the engineered guide RNA side of the guide-target RNA scaffold. An asymmetrical internal loop of the present disclosure can be formed by 300 nucleotides on the target RNA side of the guide-target RNA scaffold and 100 nucleotides on the engineered guide RNA side of the guide-target RNA scaffold. An asymmetrical internal loop of the present disclosure can be formed by 200 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 200 nucleotides on the engineered guide RNA side of the guide-target RNA scaffold. An asymmetrical internal loop of the present disclosure can be formed by 150 nucleotides on the target RNA side of the guide-target RNA scaffold and 300 nucleotides on the engineered guide RNA side of the guide-target RNA scaffold. An asymmetrical internal loop of the present disclosure can be formed by 150 nucleotides on the target RNA side of the guide-target RNA scaffold and 400 nucleotides on the engineered guide RNA side of the guide-target RNA scaffold. An asymmetrical internal loop of the present disclosure can be formed by 150 nucleotides on the target RNA side of the guide-target RNA scaffold and 500 nucleotides on the engineered guide RNA side of the guide-target RNA scaffold. An asymmetrical internal loop of the present disclosure can be formed by 150 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 150 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 150 nucleotides on the engineered guide RNA side of the guide-target RNA scaffold. An asymmetrical internal loop of the present disclosure can be formed by 300 nucleotides on the target RNA side of the guide-target RNA scaffold and 150 nucleotides on the engineered guide RNA side of the guide-target RNA scaffold. An asymmetrical internal loop of the present disclosure can be formed by 200 nucleotides on the target RNA side of the guide-target RNA scaffold and 300 nucleotides on the engineered guide RNA side of the guide-target RNA scaffold. An asymmetrical internal loop of the present disclosure can be formed by 200 nucleotides on the target RNA side of the guide-target RNA scaffold and 400 nucleotides on the engineered guide RNA side of the guide-target RNA scaffold. An asymmetrical internal loop of the present disclosure can be formed by 200 nucleotides on the target RNA side of the guide-target RNA scaffold and 500 nucleotides on the engineered guide RNA side of the guide-target RNA scaffold. An asymmetrical internal loop of the present disclosure can be formed by 200 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 200 nucleotides on the engineered guide RNA side of the guide-target RNA scaffold. An asymmetrical internal loop of the present disclosure can be formed by 500 nucleotides on the target RNA side of the guide-target RNA scaffold and 200 nucleotides on the engineered guide RNA side of the guide-target RNA scaffold. An asymmetrical internal loop of the present disclosure can be formed by 400 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 400 nucleotides on the engineered guide RNA side of the guide-target RNA scaffold. An asymmetrical internal loop of the present disclosure can be formed by 300 nucleotides on the target RNA side of the guide-target RNA scaffold and 500 nucleotides on the engineered guide RNA side of the guide-target RNA scaffold. An asymmetrical internal loop of the present disclosure can be formed by 300 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 300 nucleotides on the engineered guide RNA side of the guide-target RNA scaffold. An asymmetrical internal loop of the present disclosure can be formed by 500 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 500 nucleotides on the engineered guide RNA side of the guide-target RNA scaffold. An asymmetrical internal loop of the present disclosure can be formed by 400 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 500 nucleotides on the engineered guide RNA side of the guide-target RNA scaffold. Thus, an asymmetrical internal loop can be a structural feature formed from latent structure provided by an engineered latent guide RNA.

As described herein, a “micro-footprint” sequence refers to a sequence with latent structures that, when manifested, facilitate editing of the adenosine of a target RNA via an adenosine deaminase enzyme. A macro-footprint can serve to guide or focus an RNA editing entity (e.g., ADAR) and direct its activity towards a micro-footprint. In some embodiments, included within the micro-footprint sequence is a nucleotide that is positioned such that, when the guide RNA is hybridized to the target RNA, said nucleotide is opposite the adenosine to be edited by the ADAR enzyme and does not base pair with the adenosine to be edited. This nucleotide is referred to herein as the “mismatched position” or “mismatch” and can be a cytosine. Micro-footprint sequences as described herein have, upon hybridization of the engineered guide RNA and target RNA, at least one structural feature selected from the group consisting of: a bulge, an internal loop, a mismatch, a hairpin, and any combination thereof. Engineered guide RNAs with superior micro-footprint sequences can be selected based on their ability to facilitate editing of a specific target RNA. Engineered guide RNAs selected for their ability to facilitate editing of a specific target are capable of adopting various micro-footprint latent structures, which can vary on a target-by-target basis.

Guide RNAs of the present disclosure may further comprise a macro-footprint. In some embodiments, the macro-footprint comprises a barbell macro-footprint. A micro-footprint can serve to guide or focus an RNA editing enzyme and direct its activity towards the target adenosine to be edited. A “barbell” as described herein refers to a pair of internal loop latent structural features that manifest upon hybridization of the guide RNA to the target RNA. In some embodiments, each internal loop is positioned towards the 5′ end or the 3′ end of the guide-target RNA scaffold formed upon hybridization of the guide RNA and the target RNA. In some embodiments, each internal loop flanks opposing sides of the micro-footprint sequence. Insertion of a barbell macro-footprint sequence flanking opposing sides of the micro-footprint sequence, upon hybridization of the guide RNA to the target RNA, results in formation of barbell internal loops on opposing sides of the micro-footprint, which in turn comprises at least one structural feature that facilitates editing of a specific target RNA.

In some embodiments, the presence of barbells flanking the micro-footprint can improve one or more aspects of editing. For example, the presence of a barbell macro-footprint in addition to a micro-footprint can result in a higher amount of on target adenosine editing, relative to an otherwise comparable guide RNA lacking the barbells. Additionally, and or alternatively, the presence of a barbell macro-footprint in addition to a micro-footprint can result in a lower amount of local off-target adenosine editing, relative to an otherwise comparable guide RNA lacking the barbells. Further, while the effect of various micro-footprint structural features can vary on a target-by-target basis based on selection in a high throughput screen, the increase in the one or more aspects of editing provided by the barbell macro-footprint structures can be independent of the particular target RNA. Thus, inclusion of barbell structures can provide a facile method of improving editing of guide RNAs previously selected to facilitate editing of a target RNA of interest. For example, macro-footprints (e.g., barbell macro-footprints) and micro-footprints can provide an increased amount of on target adenosine editing relative to an otherwise comparable guide RNA lacking the barbells. In other embodiments, the presence of the barbell macro-footprint in addition to the micro-footprint can result in a lower amount of local off-target adenosine editing, relative to an otherwise comparable guide RNA, upon hybridization of the guide RNA and target RNA to form a guide-target RNA scaffold lacking the barbells.

As disclosed herein, a “macro-footprint” sequence can be positioned such that it flanks a micro-footprint sequence. Further, while a macro-footprint sequence can flank a micro-footprint sequence, additional latent structures can be incorporated that flank either end of the macro-footprint as well. In some embodiments, such additional latent structures are included as part of the macro-footprint. In some embodiments, such additional latent structures are separate, distinct, or both separate and distinct from the macro-footprint. In some embodiments, a macro-footprint sequence can comprise a barbell macro-footprint sequence comprising latent structures that, when manifested, produce a first internal loop and a second internal loop.

In some embodiments, the first internal loop of the barbell or the second internal loop of the barbell is positioned at least about 5 bases (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases) away from the A/C mismatch with respect to the base of the first internal loop or the second internal loop that is the most proximal to the A/C mismatch. In some embodiments, the first internal loop of the barbell or the second internal loop of the barbell is positioned at most about 50 bases away from the A/C mismatch (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5) with respect to the base of the first internal loop or the second internal loop that is the most proximal to the A/C mismatch.

In some embodiments, a first internal loop or a second internal loop independently comprises a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the engineered guide RNA and a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the target RNA.

As disclosed herein, a “base paired (bp) region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold. In some embodiments, a base paired region has from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

Guide RNA Expression Cassettes. A guide RNA expression cassette may comprise a promoter (e.g., any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263), a guide RNA sequence, a structural element, and a termination sequence (e.g., any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289). A guide RNA sequence may target a target RNA. In some embodiments, the target RNA encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2). Examples of engineered guide RNA expression cassettes comprising a promoter, a guide RNA sequence, a structural element, and a termination sequence are provided in TABLE 9.

TABLE 9
Exemplary Engineered Guide RNA Expression Cassettes

| | SEQ ID | |
|---|---|---|
| Target | NO: | Sequence |
| PMP22 | SEQ ID | TAACAACATAGGAGCTGTGATTGGCTGTTTTCAGCCAATCAGCA |
| | NO: 1 | CTGACTCATTTGCATAGCCTTTACAAGCGGTCACAAACTCAAGA |
| | | AACGAGCGGTTTTAATAGTCTTTTAGAATATTGTTTATCGAACCG |
| | | AATAAGGAACTGTGCTTTGTGATTCACATATCAGTGGAGGGGTG |
| | | TGGAAATGGCACCTTGATCTCACCCTCATCGAAAGTGGAGTTGA |
| | | TGTCCTTCCCTGGCTCGCTACAGACGCACTTCCGCGACCGCACCA |
| | | GCACCGCGACGTGGAGGACGATGATACTCAGCAACAGGAGGAG |
| | | CCCACTGGCGGCAAGTTCTGCTCAGCGGAGTTTCTGCCCGGCCA |
| | | AACAGCGTGTGGAATTTTTGGAGCAGGTTTTCTGACTTCGGTCGG |
| | | AAAACCCCTCCCAATTTCACTGGTCTACAATGAAAGCAAAACAG |
| | | TTCTCTTCCCCGCTCCCCGGTGTGTGAGAGGGGCTTTGATCCTTC |
| | | TCTGGTTTCCTAGGAAACGCGTATGTG |
| PMP22 | SEQ ID | TAACAACATAGGAGCTGTGATTGGCTGTTTTCAGCCAATCAGCA |
| | NO: 2 | CTGACTCATTTGCATAGCCTTTACAAGCGGTCACAAACTCAAGA |
| | | AACGAGCGGTTTTAATAGTCTTTTAGAATATTGTTTATCGAACCG |
| | | AATAAGGAACTGTGCTTTGTGATTCACATATCAGTGGAGGGGTG |
| | | TGGAAATGGCACCTTGATAAGTCACCATGAGTGTAAAGGGAGTT |
| | | GATGTCCTTCCCTGGCTCGCTACAGACGCACTTCCGCGACCGCAC |
| | | CAGCACCGCGACGTGGAGGACGATGATACTCAGCAACAGGAGG |
| | | AGCCCACTGGCGGCAAGTTCTGCTCAGCGGAGTTTCTGCCCGGC |
| | | CAAACAGCGTGTGGAATTTTTGGAGCAGGTTTTCTGACTTCGGTC |
| | | GGAAAACCCCTCCCAATTTCACTGGTCTACAATGAAAGCAAAAC |
| | | AGTTCTCTTCCCCGCTCCCCGGTGTGTGAGAGGGGCTTTGATCCT |
| | | TCTCTGGTTTCCTAGGAAACGCGTATGTG |
| PMP22 | SEQ ID | TAACAACATAGGAGCTGTGATTGGCTGTTTTCAGCCAATCAGCA |
| | NO: 3 | CTGACTCATTTGCATAGCCTTTACAAGCGGTCACAAACTCAAGA |
| | | AACGAGCGGTTTTAATAGTCTTTTAGAATATTGTTTATCGAACCG |
| | | AATAAGGAACTGTGCTTTGTGATTCACATATCAGTGGAGGGGTG |
| | | TGGAAATGGCACCTTGATCTCACCCTCATCGAAAGTGGAGTTGA |
| | | TGTCCTTCCCTGGCTCGCTACAGACGCACTTCCGCGACCGCACCA |
| | | GCACCGCGACGTGGAGGACGATGATACTCAGCAACAGGAGGAG |
| | | CCCACTGGCGGCAAGTTCTGCTCAGCGGAGTTTCTGCCCGGCCA |
| | | AACAGCGTGTGGAATTTTTGGAGCAGGTTTTCTGACTTCGGTCGG |
| | | AAAACCCCTCCCAATTTCACTGGTTTCAAAAACAGAAAAACAGT |
| | | TCTCGTTTCAAAAACAGATTCCCCGCTCCCCGGTGTGTGAGAGG |
| | | GGCTTTGATCCTTCTCTGGTTTCCTAGGAAACGCGTATGTG |
| PMP22 | SEQ ID | TAACAACATAGGAGCTGTGATTGGCTGTTTTCAGCCAATCAGCA |
| | NO: 4 | CTGACTCATTTGCATAGCCTTTACAAGCGGTCACAAACTCAAGA |
| | | AACGAGCGGTTTTAATAGTCTTTTAGAATATTGTTTATCGAACCG |
| | | AATAAGGAACTGTGCTTTGTGATTCACATATCAGTGGAGGGGTG |
| | | TGGAAATGGCACCTTGATAAGTCACCATGAGTGTAAAGGGAGTT |
| | | GATGTCCTTCCCTGGCTCGCTACAGACGCACTTCCGCGACCGCAC |
| | | CAGCACCGCGACGTGGAGGACGATGATACTCAGCAACAGGAGG |
| | | AGCCCACTGGCGGCAAGTTCTGCTCAGCGGAGTTTCTGCCCGGC |
| | | CAAACAGCGTGTGGAATTTTTGGAGCAGGTTTTCTGACTTCGGTC |
| | | GGAAAACCCCTCCCAATTTCACTGGTTTCAAAAACAGAAAAACA |
| | | GTTCTCTTCCCCGCTCCCCGGTGTGTGAGAGGGGCTTTGATCCTT |
| | | CTCTGGTTTCCTAGGAAACGCGTATGTG |
| PMP22 | SEQ ID | TAACAACATAGGAGCTGTGATTGGCTGTTTTCAGCCAATCAGCA |
| | NO: 5 | CTGACTCATGCAAATCAAGAGAAATGCAAATAGCCTTTACAAGC |
| | | GGTCACAAACTCAAGAAACGAGCGGTTTTAATAGTCTTTTAGAA |
| | | TATTGTTTATCGAACCGAATAAGGAACTGTGCTTTGTGATTCACA |
| | | TATCAGTGGAGGGGTGTGGAAATGGCACCTTGATAAGTCACCAT |
| | | GAGTGTAAAGGGAGTTGATGTCCTTCCCTGGCTCGCTACAGACG |
| | | CACTTCCGCGACCGCACCAGCACCGCGACGTGGAGGACGATGAT |
| | | ACTCAGCAACAGGAGGAGCCCACTGGCGGCAAGTTCTGCTCAGC |
| | | GGAGTTTCTGCCCGGCCAAACAGCGTGTGGAATTTTTGGAGCAG |
| | | GTTTTCTGACTTCGGTCGGAAAACCCCTCCCAATTTCACTGGTTT |
| | | CAAAAACAGAAAAACAGTTCTCTTCCCCGCTCCCCGGTGTGTGA |
| | | GAGGGGCTTTGATCCTTCTCTGGTTTCCTAGGAAACGCGTATGTG |
| SNCA | SEQ ID | TAACAACATAGGAGCTGTGATTGGCTGTTTTCAGCCAATCAGCA |
| | NO: 6 | CTGACTCATTTGCATAGCCTTTACAAGCGGTCACAAACTCAAGA |
| | | AACGAGCGGTTTTAATAGTCTTTTAGAATATTGTTTATCGAACCG |
| | | AATAAGGAACTGTGCTTTGTGATTCACATATCAGTGGAGGGGTG |
| | | TGGAAATGGCACCTTGATCTCACCCTCATCGAAAGTGGAGTTGA |
| | | TGTCCTTCCCTGGCTCGCTACAGACGCACTTCCGCGACCGGCCAC |
| | | AACTCCCTCCTTGGCCTTTGAAAGTCCTTTCATGAATACATCCAC |
| | | GGCTAATGAATTCCTTTACACCACACTGGAAAACATAAAATACA |
| | | CTTTGAGTGGAATTTTTGGAGCAGGTTTTCTGACTTCGGTCGGAA |
| | | AACCCCTCCCAATTTCACTGGTCTACAATGAAAGCAAAACAGTT |
| | | CTCTTCCCCGCTCCCCGGTGTGTGAGAGGGGCTTTGATCCTTCTC |
| | | TGGTTTCCTAGGAAACGCGTATGTG |
| SNCA | SEQ ID | TAAGGACCAGCTTCTTTGGGAGAGAACAGACGCAGGGGCGGGA |
| | NO: 7 | GGGAAAAAGGGAGAGGCAGACGTCACTTCCTCTTGGCGACTCTG |
| | | GCAGCAGATTGGTCGGTTGAGTGGCAGAAAGGCAGACGGGGAC |
| | | TGGGCAAGGCACTGTCGGTGACATCACGGACAGGGCGACTTCTA |
| | | TGTAGATGAGGCAGCGCAGAGGCTGCTGCTTCGCCACTTGCTGC |
| | | TTCGCCACGAAGGGAGTTCCCGTGCCCTGGGAGCGGGTTCAGGA |
| | | CCGCTGATCGGAAGTGAGAATCCCAGCTGTGTGTCAGGGCTGGA |
| | | AAGGGCTCGGGAGTGCGCGGGGCAAGTGACCGTGTGTGTAAAG |
| | | AGTGAGGCGTATGAGGCTGTGTCGGGGCAGAGCCCGAAGATCTC |
| | | ACCGGCCACAACTCCCTCCTTGGCCTTTGAAAGTCCTTTCATGAA |
| | | TACATCCACGGCTAATGAATTCCTTTACACCACACTGGAAAACA |
| | | TAAAATACACTTTGAGTGGAATTTTTGGAGCAGGTTTTCTGACTT |
| | | CGGTCGGAAAACCCCTCCCAATTTCACTGGTCTACAATGAAAGC |
| | | AAAACAGTTCTCTTCCCCGCTCCCCGGTGTGTGAGAGGGGCTTTG |
| | | ATCCTTCTCTGGTTTCCTAGGAAACGCGTATGTG |
| SNCA | SEQ ID | TTAACAACAACGAAGGGGCTGTGACTGGCTGCTTTCTCAACCAA |
| | NO: 8 | TCAGCACCGAACTCATTTGCATGGGCTGAGAACAAATGTTCGCG |
| | | AACTCTAGAAATGAATGACTTAAGTAAGTTCCTTAGAATATTATT |
| | | TTTCCTACTGAAAGTTACCACATGCGTCGTTGTTTATACAGTAAT |
| | | AGGAACAAGAAAAAAGTCACCTAAGCTCACCCTCATCAATTGTG |
| | | GAGTTCCTTTATATCCCATCTTCTCTCCAAACACATACGCAGACC |
| | | GGCCACAACTCCCTCCTTGGCCTTTGAAAGTCCTTTCATGAATAC |
| | | ATCCACGGCTAATGAATTCCTTTACACCACACTGGAAAACATAA |
| | | AATACACTTTGAGTGGAATTTTTGGAGCAGGTTTTCTGACTTCGG |
| | | TCGGAAAACCCCTCCCAATTTCACTGGTCTACAATGAAAGCAAA |
| | | ACAGTTCTCTTCCCCGCTCCCCGGTGTGTGAGAGGGGCTTTGATC |
| | | CTTCTCTGGTTTCCTAGGAAACGCGTATGTG |
| SNCA | SEQ ID | TAACAACATAGGAGCTGTGATTGGCTGTTTTCAGCCAATCAGCA |
| | NO: 9 | CTGACTCATTTGCATAGCCTTTACAAGCGGTCACAAACTCAAGA |
| | | AACGAGCGGTTTTAATAGTCTTTTAGAATATTGTTTATCGAACCG |
| | | AATAAGGAACTGTGCTTTGTGATTCACATATCAGTGGAGGGGTG |
| | | TGGAAATGGCACCTTGATAAGTCACCATGAGTGTAAAGGGAGTT |
| | | GATGTCCTTCCCTGGCTCGCTACAGACGCACTTCCGCGACCGGCC |
| | | ACAACTCCCTCCTTGGCCTTTGAAAGTCCTTTCATGAATACATCC |
| | | ACGGCTAATGAATTCCTTTACACCACACTGGAAAACATAAAATA |
| | | CACTTTGAGTGGAATTTTTGGAGCAGGTTTTCTGACTTCGGTCGG |
| | | AAAACCCCTCCCAATTTCACTGGTCTACAATGAAAGCAAAACAG |
| | | TTCTCTTCCCCGCTCCCCGGTGTGTGAGAGGGGCTTTGATCCTTC |
| | | TCTGGTTTCCTAGGAAACGCGTATGTG |
| SNCA | SEQ ID | TAACAACATAGGAGCTGTGATTGGCTGTTTTCAGCCAATCAGCA |
| | NO: 10 | CTGACTCATTTGCATAGCCTTTACAAGCGGTCACAAACTCAAGA |
| | | AACGAGCGGTTTTAATAGTCTTTTAGAATATTGTTTATCGAACCG |
| | | AATAAGGAACTGTGCTTTGTGATTCACATATCAGTGGAGGGGTG |
| | | TGGAAATGGCACCTTGATCTCACCCTCATCGAAAGTGGAGTTGA |
| | | TGTCCTTCCCTGGCTCGCTACAGACGCACTTCCGCGACCGGCCAC |
| | | AACTCCCTCCTTGGCCTTTGAAAGTCCTTTCATGAATACATCCAC |
| | | GGCTAATGAATTCCTTTACACCACACTGGAAAACATAAAATACA |
| | | CTTTGAGTGGAATTTTTGGAGCAGGTTTTCTGACTTCGGTCGGAA |
| | | AACCCCTCCCAATTTCACTGGTTTCAAAAACAGAAAAACAGTTC |
| | | TCGTTTCAAAAACAGATTCCCCGCTCCCCGGTGTGTGAGAGGGG |
| | | CTTTGATCCTTCTCTGGTTTCCTAGGAAACGCGTATGTG |
| SNCA | SEQ ID | TAACAACATAGGAGCTGTGATTGGCTGTTTTCAGCCAATCAGCA |
| | NO: 11 | CTGACTCATTTGCATAGCCTTTACAAGCGGTCACAAACTCAAGA |
| | | AACGAGCGGTTTTAATAGTCTTTTAGAATATTGTTTATCGAACCG |
| | | AATAAGGAACTGTGCTTTGTGATTCACATATCAGTGGAGGGGTG |
| | | TGGAAATGGCACCTTGATAAGTCACCATGAGTGTAAAGGGAGTT |
| | | GATGTCCTTCCCTGGCTCGCTACAGACGCACTTCCGCGACCGGCC |
| | | ACAACTCCCTCCTTGGCCTTTGAAAGTCCTTTCATGAATACATCC |
| | | ACGGCTAATGAATTCCTTTACACCACACTGGAAAACATAAAATA |
| | | CACTTTGAGTGGAATTTTTGGAGCAGGTTTTCTGACTTCGGTCGG |
| | | AAAACCCCTCCCAATTTCACTGGTTTCAAAAACAGAAAAACAGT |
| | | TCTCTTCCCCGCTCCCCGGTGTGTGAGAGGGGCTTTGATCCTTCT |
| | | CTGGTTTCCTAGGAAACGCGTATGTG |
| SNCA | SEQ ID | TAACAACATAGGAGCTGTGATTGGCTGTTTTCAGCCAATCAGCA |
| | NO: 12 | CTGACTCATGCAAATCAAGAGAAATGCAAATAGCCTTTACAAGC |
| | | GGTCACAAACTCAAGAAACGAGCGGTTTTAATAGTCTTTTAGAA |
| | | TATTGTTTATCGAACCGAATAAGGAACTGTGCTTTGTGATTCACA |
| | | TATCAGTGGAGGGGTGTGGAAATGGCACCTTGATAAGTCACCAT |
| | | GAGTGTAAAGGGAGTTGATGTCCTTCCCTGGCTCGCTACAGACG |
| | | CACTTCCGCGACCGGCCACAACTCCCTCCTTGGCCTTTGAAAGTC |
| | | CTTTCATGAATACATCCACGGCTAATGAATTCCTTTACACCACAC |
| | | TGGAAAACATAAAATACACTTTGAGTGGAATTTTTGGAGCAGGT |
| | | TTTCTGACTTCGGTCGGAAAACCCCTCCCAATTTCACTGGTTTCA |
| | | AAAACAGAAAAACAGTTCTCTTCCCCGCTCCCCGGTGTGTGAGA |
| | | GGGGCTTTGATCCTTCTCTGGTTTCCTAGGAAACGCGTATGTG |
| SERPINA1 | SEQ ID | TAACAACATAGGAGCTGTGATTGGCTGTTTTCAGCCAATCAGCA |
| | NO: 59 | CTGACTCATTTGCATAGCCTTTACAAGCGGTCACAAACTCAAGA |
| | | AACGAGCGGTTTTAATAGTCTTTTAGAATATTGTTTATCGAACCG |
| | | AATAAGGAACTGTGCTTTGTGATTCACATATCAGTGGAGGGGTG |
| | | TGGAAATGGCACCTTGATAAGTCACCATGAGTGTAAAGGGAGTT |
| | | GATGTCCTTCCCTGGCTCGCTACAGACGCACTTCCGCGACCGTAG |
| | | ACATGGGTATGGCCTCTAATTTGTAGGCCCCAGCAGCTTCAGTCC |
| | | CTTACTCGTCGTACCAGAGCACAGCCAGTCGTATGCACGGCGTG |
| | | GAATTTTTGGAGCAGGTTTTCTGACTTCGGTCGGAAAACCCCTCC |
| | | CAATTTCACTGGTTTCAAAAACAGAAAAACAGTTCTCTTCCCCGC |
| | | TCCCCGGTGTGTGAGAGGGGCTTTGATCCTTCTCTGGTTTCCTAG |
| | | GAAACGCGTATGTG |

In some embodiments, an engineered guide RNA expression cassette may have at least about 70%, at least about 75%, at least about 80%, at least about 83%, at least about 85%, at least about 87%, at least about 90%, at least about 91%, at least about 92%, at least about 93%, at least about 94%, at least about 95%, at least about 96% at least about 97%, at least about 98%, at least about 99%, or about 100% sequence identity to any of SEQ ID NO: 1-SEQ ID NO: 12 or SEQ ID NO: 59.

An engineered guide RNA expression cassette may comprise a promoter (e.g., any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263), a guide RNA sequence, a structural element, and a termination sequence (e.g., any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289).

For example, the engineered guide RNA expression cassette of SEQ ID NO: 1 comprises a promoter of SEQ ID NO: 15, a PMP22 guide RNA sequence of SEQ ID NO: 1273, and a termination sequence of SEQ ID NO: 1243. For example, the engineered guide RNA expression cassette of SEQ ID NO: 2 comprises a promoter of SEQ ID NO: 16, a PMP22 guide RNA sequence of SEQ ID NO: 1273, and a termination sequence of SEQ ID NO: 1243. For example, the engineered guide RNA expression cassette of SEQ ID NO: 3 comprises a promoter of SEQ ID NO: 15, a PMP22 guide RNA sequence of SEQ ID NO: 1273, and a termination sequence of SEQ ID NO: 1275. For example, the engineered guide RNA expression cassette of SEQ ID NO: 4 comprises a promoter of SEQ ID NO: 16, a PMP22 guide RNA sequence of SEQ ID NO: 1273, and a termination sequence of SEQ ID NO: 60. For example, the engineered guide RNA expression cassette of SEQ ID NO: 5 comprises a promoter of SEQ ID NO: 17, a PMP22 guide RNA sequence of SEQ ID NO: 1273, and a termination sequence of SEQ ID NO: 60.

For example, the engineered guide RNA expression cassette of SEQ ID NO: 6 comprises a promoter of SEQ ID NO: 15, a SNCA guide RNA sequence of SEQ ID NO: 1274, and a termination sequence of SEQ ID NO: 1243. For example, the engineered guide RNA expression cassette of SEQ ID NO: 7 comprises a promoter of SEQ ID NO: 13, a SNCA guide RNA sequence of SEQ ID NO: 1290, and a termination sequence of SEQ ID NO: 1243. For example, the engineered guide RNA expression cassette of SEQ ID NO: 8 comprises a promoter of SEQ ID NO: 14, a SNCA guide RNA sequence of SEQ ID NO: 1274, and a termination sequence of SEQ ID NO: 1243. For example, the engineered guide RNA expression cassette of SEQ ID NO: 9 comprises a promoter of SEQ ID NO: 16, a SNCA guide RNA sequence of SEQ ID NO: 1274, and a termination sequence of SEQ ID NO: 1243. For example, the engineered guide RNA expression cassette of SEQ ID NO: 10 comprises a promoter of SEQ ID NO: 15, a SNCA guide RNA sequence of SEQ ID NO: 1274, and a termination sequence of SEQ ID NO: 1275. For example, the engineered guide RNA expression cassette of SEQ ID NO: 11 comprises a promoter of SEQ ID NO: 16, a SNCA guide RNA sequence of SEQ ID NO: 1274, and a termination sequence of SEQ ID NO: 60. For example, the engineered guide RNA expression cassette of SEQ ID NO: 12 comprises a promoter of SEQ ID NO: 17, a SNCA guide RNA sequence of SEQ ID NO: 1274, and a termination sequence of SEQ ID NO: 60.

For example, the engineered guide RNA expression cassette of SEQ ID NO: 59 comprises a promoter of SEQ ID NO: 16, a SERPINA 1 guide RNA sequence of SEQ ID NO: 61, and a termination sequence of SEQ ID NO: 60.

### Additional Engineered Guide RNA Components

The present disclosure provides for engineered guide RNAs with additional structural features and components. For example, an engineered guide RNA described herein can be circular. In another example, an engineered guide RNA described herein can comprise a U7, an SmOPT sequence, or a combination of both sequences.

In some cases, an engineered guide RNA can be circularized. In some cases, an engineered guide RNA provided herein can be circularized or in a circular configuration. In some aspects, an at least partially circular guide RNA lacks a 5′ hydroxyl or a 3′ hydroxyl.

In some examples, an engineered guide RNA can comprise a backbone comprising a plurality of sugar and phosphate moieties covalently linked together. In some examples, a backbone of an engineered guide RNA can comprise a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an engineered guide RNA can lack a 5′ reducing hydroxyl, a 3′ reducing hydroxyl, or both, capable of being exposed to a solvent. In some embodiments, a backbone of an engineered guide can lack a 5′ reducing hydroxyl, a 3′ reducing hydroxyl, or both, capable of being exposed to nucleases. In some embodiments, a backbone of an engineered guide can lack a 5′ reducing hydroxyl, a 3′ reducing hydroxyl, or both, capable of being exposed to hydrolytic enzymes. In some instances, a backbone of an engineered guide can be represented as a polynucleotide sequence in a circular 2-dimensional format with one nucleotide after the other. In some instances, a backbone of an engineered guide can be represented as a polynucleotide sequence in a looped 2-dimensional format with one nucleotide after the other. In some cases, a 5′ hydroxyl, a 3′ hydroxyl, or both, can be joined through a phosphorus-oxygen bond. In some cases, a 5′ hydroxyl, a 3′ hydroxyl, or both, can be modified into a phosphoester with a phosphorus-containing moiety.

As described herein, an engineered guide can comprise a circular structure. An engineered polynucleotide can be circularized from a precursor engineered polynucleotide. Such a precursor engineered polynucleotide can be a precursor engineered linear polynucleotide. In some cases, a precursor engineered linear polynucleotide can be a precursor for a circular engineered guide RNA. For example, a precursor engineered linear polynucleotide can be a linear mRNA transcribed from a plasmid, which can be configured to circularize within a cell using the techniques described herein. A precursor engineered linear polynucleotide can be constructed with domains such as a ribozyme domain and a ligation domain that allow for circularization when inserted into a cell. A ribozyme domain can include a domain that is capable of cleaving the linear precursor RNA at specific sites (e.g., adjacent to the ligation domain). A precursor engineered linear polynucleotide can comprise, from 5′ to 3′: a 5′ ribozyme domain, a 5′ ligation domain, a circularized region, a 3′ ligation domain, and a 3′ ribozyme domain. In some cases, a circularized region can comprise a guide RNA described herein. In some cases, the precursor polynucleotide can be specifically processed at both sites by the 5′ and the 3′ ribozymes, respectively, to free exposed ends on the 5′ and 3′ ligation domains. The free exposed ends can be ligation competent, such that the ends can be ligated to form a mature circularized structure. For instance, the free ends can include a 5′-OH and a 2′, 3′-cyclic phosphate that are ligated via RNA ligation in the cell. The linear polynucleotide with the ligation and ribozyme domains can be transfected into a cell where it can circularize via endogenous cellular enzymes. In some cases, a polynucleotide can encode an engineered guide RNA comprising the ribozyme and ligation domains described herein, which can circularize within a cell. For example, PCT/US2021/034301 provides a description of circular guide RNAs and their structures, sequences of circular guide RNAs, and methods of engineering circularized polynucleotide domains, and each of these descriptions in PCT/US2021/034301 is herein incorporated by reference.

An engineered polynucleotide as described herein (e.g., a circularized guide RNA) can include spacer domains. As described herein, a spacer domain can refer to a domain that provides space between other domains. A spacer domain can be used to between a region to be circularized and flanking ligation sequences to increase the overall size of the mature circularized guide RNA. Where the region to be circularized includes a targeting domain as described herein that is configured to associate to a target sequence, the addition of spacers can provide improvements (e.g., increased specificity, enhanced editing efficiency, etc.) for the engineered polynucleotide to the target polynucleotide, relative to a comparable engineered polynucleotide that lacks a spacer domain. In some instances, the spacer domain is configured to not hybridize with the target RNA. In some embodiments, a precursor engineered polynucleotide or a circular engineered guide, can comprise, in order of 5′ to 3′: a first ribozyme domain; a first ligation domain; a first spacer domain; a targeting domain that can be at least partially complementary to a target RNA, a second spacer domain, a second ligation domain, and a second ribozyme domain. In some cases, the first spacer domain, the second spacer domain, or both are configured to not bind to the target RNA when the targeting domain binds to the target RNA.

The compositions and methods of the present disclosure provide engineered polynucleotides encoding for guide RNAs that are operably linked to a portion of a small nuclear ribonucleic acid (snRNA) sequence. The engineered polynucleotide can include at least a portion of a small nuclear ribonucleic acid (snRNA) sequence. The U7 and U1 small nuclear RNAs, whose natural role is in spliceosomal processing of pre-mRNA, have for decades been re-engineered to alter splicing at desired disease targets. Replacing a portion of the U7 snRNA which naturally hybridizes to the spacer element of histone pre-mRNA (e.g., the first 18 nucleotides of the U7 snRNA) with a short targeting (or antisense) sequence of a disease gene, may redirect the splicing machinery to alter splicing around that target site. Furthermore, converting the wild type U7 Sm-domain binding site to an optimized consensus Sm-binding sequence (SmOPT) can increase the expression level, activity, and subcellular localization of the artificial antisense-engineered U7 snRNA. Many subsequent groups have adapted this modified U7 SmOPT snRNA chassis with antisense sequences of other genes to recruit spliceosomal elements and modify RNA splicing for additional disease targets.

An snRNA is a class of small RNA molecules found within the nucleus of eukaryotic cells. They are involved in a variety of important processes such as RNA splicing (removal of introns from pre-mRNA), regulation of transcription factors (7SK RNA) or RNA polymerase II (B2 RNA), and maintaining the telomeres. They are always associated with specific proteins, and the resulting RNA-protein complexes are referred to as small nuclear ribonucleoproteins (snRNP) or sometimes as snurps. There are many snRNAs, which are denominated U1, U2, U3, U4, U5, U6, U7, U8, U9, and U10.

The snRNA of the U7 type is normally involved in the maturation of histone mRNA. This snRNA has been identified in a great number of eukaryotic species (56 so far) and the U7 snRNA of each of these species should be regarded as equally convenient for this disclosure.

Wild type U7 snRNA includes a stem-loop structure, the U7-specific Sm sequence, and a sequence antisense to the 3′ end of histone pre-mRNA.

In addition to the SmOPT domain, U7 comprises a sequence antisense to the 3′ end of histone pre-mRNA. When this sequence is replaced by a targeting sequence that is antisense to another target pre-mRNA, U7 is redirected to the new target pre-mRNA. Accordingly, the stable expression of modified U7 snRNAs containing the SmOPT domain and a targeting antisense sequence has resulted in specific alteration of mRNA splicing. While AAV-2/1 based vectors expressing an appropriately modified murine U7 gene along with its natural promoter and 3′ elements have enabled high efficiency gene transfer into the skeletal muscle and complete dystrophin rescue by covering and skipping mouse Dmd exon 23, the engineered polynucleotides as described herein (whether directly administered or administered via, for example, AAV vectors) can facilitate editing of target RNA by a deaminase.

The engineered polynucleotide can comprise at least in part an snRNA sequence. The snRNA sequence can be U1, U2, U3, U4, U5, U6, U7, U8, U9, or a U10 snRNA sequence.

In some instances, an engineered polynucleotide that comprises at least a portion of an snRNA sequence (e.g., an snRNA promoter, an snRNA hairpin, and the like) can have superior properties for treating or preventing a disease or condition, relative to a comparable polynucleotide lacking such features. For example, as described herein an engineered polynucleotide that comprises at least a portion of an snRNA sequence can facilitate exon skipping of an exon at a greater efficiency than a comparable polynucleotide lacking such features. Further, as described herein an engineered polynucleotide that comprises at least a portion of an snRNA sequence can facilitate an editing of a base of a nucleotide in a target RNA (e.g., a pre-mRNA or a mature RNA) at a greater efficiency than a comparable polynucleotide lacking such features. Promoters and snRNA components are described in PCT/US2021/028618 and PCT/US2022/078801, and each of these descriptions in PCT/US2021/028618 and PCT/US2022/078801 are herein incorporated by reference.

Disclosed herein are engineered RNAs comprising (a) an engineered guide RNA as described herein, and (b) a U7 snRNA hairpin sequence, a SmOPT sequence, or a combination thereof. In some embodiments, the U7 hairpin comprises a human U7 Hairpin sequence, or a mouse U7 hairpin sequence. In some cases, a human U7 hairpin sequence comprises TAGGCTTTCTGGCTTTTTACCGGAAAGCCCCT (SEQ ID NO: 52) or RNA: UAGGCUUUCUGGCUUUUUACCGGAAAGCCCCU (SEQ ID NO: 53). In some cases, a mouse U7 hairpin sequence comprises CAGGTTTTCTGACTTCGGTCGGAAAACCCCT (SEQ ID NO: 54) or RNA: CAGGUUUUCUGACUUCGGUCGGAAAACCCCU (SEQ ID NO: 55). In some embodiments, the SmOPT sequence has a sequence of AATTTTTGGAG (SEQ ID NO: 56) or RNA: AAUUUUUGGAG (SEQ ID NO: 57). In some embodiments, an RNA payload may comprise a guide RNA, a U7 hairpin sequence (e.g., a human or a mouse U7 hairpin sequence), an SmOPT sequence, or a combination thereof. For example, an RNA payload may comprise a sequence of AATTTTTGGAGCAGGTTTTCTGACTTCGGTCGGAAAACCCCTCCCAATTTCACTGGT CTACAATGAAAGCAAAACAGTTCTCTTCCCCGCTCCCCGGTGTGTGAGAGGGGCTTT GATCCTTCTCTGGTTTCCTAGGAAACGCGTATGTG (SEQ ID NO: 58). In some cases, a combination of a U7 hairpin sequence and a SmOPT sequence can comprise a SmOPT U7 hairpin sequence, wherein the SmOPT sequence is linked to the U7 sequence. In some cases, a U7 hairpin sequence, an SmOPT sequence, or a combination thereof is downstream (e.g., 3′) of the engineered guide RNA disclosed herein.

### Guide RNA Payloads for DNA Editing

The expression cassettes described herein may be used to enhance expression of RNA components for site-specific, selective editing of a target DNA via a DNA editing entity or a biologically active fragment thereof. An RNA component for site-specific DNA editing may comprise a guide RNA, a transactivating CRISPR RNA (tracrRNA), a single guide RNA, or engineered polynucleotides encoding the same. An engineered guide RNA, as described herein, may comprise a sequence with complementarity to a target DNA described herein. As such, a guide RNA can be engineered to site-specifically/selectively target and hybridize to a particular target DNA, thus facilitating editing of specific nucleotide in the target DNA via a DNA editing entity or a biologically active fragment thereof. DNA editing may be facilitated by a nuclease, such as a Cas nuclease. In some embodiments, the Cas nuclease may be a Cas9, a Cas12, or a Cas14.

In some embodiments, an engineered guide RNA hybridizes to a sequence of the target DNA. In some embodiments, part of the engineered guide RNA hybridizes to the sequence of the target DNA. The part of the engineered guide RNA that hybridizes to the target DNA is of sufficient complementary to the sequence of the target DNA for hybridization to occur. In some embodiments, the guide RNA may comprise a sequence having at least about 95%, at least about 96%, at least about 97%, at least about 98%, at least about 99%, or about 100% sequence complementarity to a target DNA. A guide RNA encoded by an expression cassette of the present disclosure may comprise a length of from about 15 to about 70 nucleotides, from about 40 to about 70 nucleotides, or from about 70 to about 100 nucleotides. In some embodiments, the region of the guide RNA that hybridizes to the target may comprise a length of from about 18 to about 44 nucleotides.

In some examples, an engineered guide RNA can facilitate editing of a base of a nucleotide of in a target sequence of a target DNA that results in modulating the expression of a gene encoded by the target DNA. In some instances, modulation can be increased or decrease expression of the gene. In some cases, an engineered guide can be configured to facilitate an editing of a base of a nucleotide or polynucleotide of a region of an DNA by a DNA editing entity (e.g., a Cas nuclease).

In some embodiments, the expression cassettes described herein may be used to enhance expression of transactivating crRNAs (tracrRNAs) and engineered polynucleotides encoding the same for editing of a target DNA via a DNA editing entity or a biologically active fragment thereof. The tracrRNA may bind to and activate a DNA editing enzyme (e.g., a Cas nuclease). A tracrRNA encoded by an expression cassette of the present disclosure may comprise a length of from about 75 to about 100 nucleotides.

In some embodiments, the expression cassettes described herein may be used to enhance expression of a single guide RNA and engineered polynucleotides encoding the same for editing of a target DNA via a DNA editing entity or a biologically active fragment thereof. The single guide RNA may comprise a region that binds to and activates a DNA editing enzyme (e.g., a Cas nuclease) and a region that hybridizes to the sequence of the target DNA. The part of the single guide RNA that hybridizes to the target DNA is of sufficient complementary to the sequence of the target DNA for hybridization to occur. In some embodiments, the single guide RNA may comprise a sequence having at least about 95%, at least about 96%, at least about 97%, at least about 98%, at least about 99%, or about 100% sequence complementarity to a target DNA. A single guide RNA encoded by an expression cassette of the present disclosure may comprise a length of from about 80 to about 120 nucleotides. In some embodiments, the region of the single guide RNA that hybridizes to the target may comprise a length of from about 18 to about 44 nucleotides.

### Other RNA-Targeting Oligonucleotides

The expression cassettes described herein may be used to enhance expression of other engineered RNA-targeting oligonucleotides, including antisense oligonucleotides, siRNAs, shRNAs, and miRNAs, and engineered polynucleotides encoding the same that hybridizes to a target RNA (e.g., a target mRNA or a target pre-mRNA). An engineered oligonucleotide, as described herein, may comprise a targeting domain with complementarity to a target RNA described herein. As such, an oligonucleotide can be engineered to target and hybridize to a particular target RNA, thus altering expression of a polypeptide encoded by the target RNA.

In some embodiments, the engineered oligonucleotide (e.g., antisense oligonucleotide, siRNA, shRNA, or miRNA) of the present disclosure hybridizes to a sequence of the target RNA. In some embodiments, part of the engineered oligonucleotide (e.g., a targeting domain) hybridizes to the sequence of the target RNA. The part of the engineered oligonucleotide that hybridizes to the target RNA is of sufficient complementary to the sequence of the target RNA for hybridization to occur. A targeting sequence can also be referred to as a “targeting domain” or a “targeting region.” In some embodiments, binding of the engineered oligonucleotide to the target RNA may recruit additional components, such as RISC components.

### Therapeutic Applications

The expression cassettes of the present disclosure encoding an RNA payload under transcriptional control of an engineered promoter may have a variety of therapeutic applications. The engineered promoters described herein may facilitate the therapeutic use by increasing payload expression and enhancing a therapeutic effect produced by the payload. For example, increased guide RNA payload expression may enhance editing efficiency of a target DNA or RNA. In another example, increased antisense oligonucleotide expression may enhance target knockdown efficiency.

### RNA Editing

RNA editing can refer to a process by which RNA can be enzymatically modified post synthesis at specific nucleosides. RNA editing can comprise any one of an insertion, deletion, or substitution of a nucleotide(s). Examples of RNA editing include chemical modifications, such as pseudouridylation (the isomerization of uridine residues) and deamination (removal of an amine group from: cytidine to give rise to uridine, or C-to-U editing; or from adenosine to inosine, or A-to-I editing). RNA editing can be used to correct mutations (e.g., correction of a missense mutation) to restore protein expression, or to introduce mutations or edit coding or non-coding regions of RNA to inhibit RNA translation and effect protein knockdown. An expression cassette of the present disclosure may be used to express an engineered guide RNA to facilitate RNA editing by an RNA entity (e.g., an adenosine Deaminase Acting on RNA (ADAR)) or biologically active fragments thereof.

Described herein are engineered guide RNAs that facilitate RNA editing by an RNA editing entity (e.g., an adenosine Deaminase Acting on RNA (ADAR)) or biologically active fragments thereof. In some instances, ADARs can be enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR enzymes share a common domain architecture comprising a variable number of amino-terminal dsRNA binding domains (dsRBDs) and a single carboxy-terminal catalytic deaminase domain. Human ADARs possess two or three dsRBDs. Evidence suggests that ADARs can form homodimer as well as heterodimer with other ADARs when bound to double-stranded RNA, however it can be currently inconclusive if dimerization is needed for editing to occur. The engineered guide RNAs disclosed herein can facilitate RNA editing by any of or any combination of the three human ADAR genes that have been identified (ADARs 1-3). ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain.

The engineered guide RNAs of the present disclosure facilitate RNA editing by endogenous ADAR enzymes. In some embodiments, exogenous ADAR can be delivered alongside the engineered guide RNAs disclosed herein to facilitate RNA editing. In some embodiments, the ADAR is human ADAR1. In some embodiments, the ADAR is human ADAR2. In some embodiments, the ADAR is human ADAR3. In some embodiments, the ADAR is human ADAR1, human ADAR2, human ADAR2, or any combination thereof.

The present disclosure, in some embodiments, provides engineered guide RNAs that facilitate edits at particular regions in a target RNA (e.g., mRNA or pre-mRNA). For example, the engineered guide RNAs disclosed herein can target a coding sequence or a non-coding sequence of an RNA. For example, a target region in a coding sequence of an RNA can be a translation initiation site (TIS). In some embodiments, the target region in a non-coding sequence of an RNA can be a polyadenylation (polyA) signal sequence.

Missense Mutations. In some embodiments, the engineered guide RNAs of the present disclosure may target a missense mutation in a target RNA sequence. The engineered guide RNAs may facilitate ADAR-mediated RNA editing of a target adenosine (A) to convert to an inosine (I), which may be read as a guanosine (G). Conversion of A to I via ADAR-mediated RNA editing may correct G to A missense mutations. For example, ADAR-mediated editing may correct a valine to isoleucine or valine to methionine mutation by converting an isoleucine codon (AUU, AUC, or AUA) or methionine codon (AUG) to a valine codon (AUA, GUC, GUU, or GUG). In another example, ADAR-mediated editing may correct a cysteine to tyrosine or mutation by converting a tyrosine codon (AUA or UAC) to a cysteine codon (UGU or UGC). Alternatively, or in addition, the engineered guide RNAs may facilitate APOBEC-mediated RNA editing of a target cytosine (C) to convert to a uracil (U). Conversion of C to U via APOBEC-mediated RNA editing may correct U to C missense mutations. Engineered guide RNAs of the present disclosure can target one or any combination of missense mutations of a target sequence (e.g., SNCA, PMP22, DUX4, LRRK2, MAPT, GRN, ABCA4, APP, SERPINA1, HEXA, CFTR, LIPA, GBA, PINK1, or MECP2).

Nonsense Mutations. In some embodiments, the engineered guide RNAs of the present disclosure may target a nonsense mutation in a target RNA sequence. The engineered guide RNAs may facilitate ADAR-mediated RNA editing of a target adenosine (A) to convert to an inosine (I), which may be read as a guanosine (G). Conversion of A to I via ADAR-mediated RNA editing may correct G to A nonsense mutations. For example, ADAR-mediated editing may correct a tryptophan to stop nonsense mutation by converting a UAG stop codon to a tryptophan codon (UGG). In another example, ADAR-mediated editing may correct a tryptophan to stop nonsense mutation by converting a UGA stop codon to a tryptophan codon (UGG). Correction of nonsense mutations via ADAR-mediated editing may increase expression of the target sequence. Engineered guide RNAs of the present disclosure can target one or any combination of missense mutations of a target sequence (e.g., SNCA, PMP22, DUX4, LRRK2, MAPT, GRN, ABCA4, APP, SERPINA1, HEXA, CFTR, LIPA, GBA, PINK1, or MECP2).

TIS. In some embodiments, the engineered guide RNAs of the present disclosure target the adenosine at a translation initiation site (TIS). The engineered guide RNAs may facilitate ADAR-mediated RNA editing of the TIS (AUG) to GUG. This results in inhibition of RNA translation and, thereby, protein knockdown. Protein knockdown can also be referred to as reduced expression of wild type protein. Engineered guide RNAs of the present disclosure can target one or any combination of the TISs of a target sequence (e.g., SNCA, PMP22, DUX4, LRRK2, MAPT, GRN, ABCA4, APP, SERPINA1, HEXA, CFTR, LIPA, GBA, PINK1, or MECP2).

3′UTR. In some embodiments, the engineered guide RNAs of the present disclosure target one or more adenosines in the 3′ untranslated region (3′UTR). In some embodiments, an engineered guide RNA facilitates ADAR-mediated RNA editing of the one or more adenosines in the 3′UTR, thereby reducing mRNA export from the nucleus and inhibiting translation, thereby resulting protein knockdown. In some embodiments, the target sequence may be SNCA, PMP22, DUX4, LRRK2, MAPT, GRN, ABCA4, APP, SERPINA1, HEXA, CFTR, LIPA, GBA, PINK1, or MECP2.

PolyA Signal Sequence. In some embodiments, the engineered guide RNAs of the present disclosure target one or more adenosines in the polyA signal sequence. In some embodiments, an engineered guide RNA facilitates ADAR-mediated RNA editing of the one or more adenosines in the polyA signal sequence, thereby resulting in disruption of RNA processing and degradation of the target mRNA and, thereby, protein knockdown. In some embodiments, a target can have one or more polyA signal sequences. In these instances, one or more engineered guide RNAs, varying in their respective sequences, of the present disclosure can be multiplexed to target adenosines in the one or more polyA signal sequences. In both cases, the engineered guide RNAs of the present disclosure facilitated ADAR-mediated RNA editing of adenosines to inosines (read as guanosines by cellular machinery) in the polyA signal sequence, resulting in protein knockdown. In some embodiments, the target sequence may be SNCA, PMP22, DUX4, LRRK2, MAPT, GRN, ABCA4, APP, SERPINA1, HEXA, CFTR, LIPA, GBA, PINK1, or MECP2.

### DNA Editing

DNA editing can refer to a process by which DNA can be enzymatically (e.g., by an RNA-guided endonuclease). DNA editing can comprise any one of an insertion, deletion, or substitution of a nucleotide(s). DNA editing can be used to correct mutations (e.g., correction of a missense mutation) to restore protein expression, or to introduce mutations or edit coding or non-coding regions of DNA to inhibit DNA transcription and effect protein knockdown. An expression cassette of the present disclosure may be used to express an engineered guide RNA to facilitate DNA editing by a DNA entity (e.g., CRISPR/Cas endonuclease) or biologically active fragments thereof. Described herein are engineered guide RNAs that facilitate DNA editing by a DNA editing entity (e.g., CRISPR/Cas endonuclease) or biologically active fragments thereof.

The engineered guide RNAs of the present disclosure may facilitate DNA editing by endogenous Cas enzymes. In some embodiments, exogenous Cas enzymes can be delivered alongside the engineered guide RNAs disclosed herein to facilitate DNA editing. In some embodiments, the Cas nuclease is Cas9. In some embodiments, the Cas nuclease is Cas12. In some embodiments, the Cas nuclease is Cas14.

The present disclosure, in some embodiments, provides engineered guide RNAs that facilitate edits at particular regions in a target DNA. For example, the engineered guide RNAs disclosed herein can target a coding sequence or a non-coding sequence of a DNA.

An engineered guide RNA of the present disclosure may recruit a CRISPR/Cas endonuclease (e.g., a Cas9 nuclease) to form a ribonucleoprotein (RNP) complex that is targeted to a particular site in a target polynucleotide (e.g., a target DNA) via base pairing between the guide RNA and a target region within the target polynucleotide. The engineered guide RNA may include a targeting sequence that is complementary to a target site of the target polynucleotide. Thus, an engineered guide RNA forms a complex with a Cas nuclease, and the guide RNA provides sequence specificity to the RNP complex via the targeting sequence. Upon recruitment to the target polynucleotide, the Cas nuclease may site-specifically edit the target polynucleotide (e.g., the target DNA). In some embodiments, the target polynucleotide may encode SNCA, PMP22, DUX4, LRRK2, MAPT, GRN, ABCA4, APP, SERPINA1, HEXA, CFTR, LIPA, GBA, PINK1, or MECP2.

### Expression Knockdown

An expression cassette of the present disclosure may be used to express an engineered RNA-targeting oligonucleotide (e.g., an antisense oligonucleotide, an siRNA, an shRNA, or a miRNA) to facilitate knockdown expression of the target RNA. In some embodiments, binding of the RNA-targeting oligonucleotide to the target RNA may recruit additional components (e.g., RISC complex components) to the target RNA that may reduce expression of a peptide encoded by the target RNA. For example, binding of an siRNA may recruit RISC and facilitate cleavage of the target RNA. In another example, binding of a miRNA or an shRNA may recruit RISC and inhibit translation of the target RNA. In some embodiments, the target RNA may encode SNCA, PMP22, DUX4, LRRK2, MAPT, GRN, ABCA4, APP, SERPINA1, HEXA, CFTR, LIPA, GBA, PINK1, or MECP2.

### Targets and Methods of Treatment

A small RNA payload, such as an engineered guide RNA, of the present disclosure can be used in a method of treating a disorder in a subject in need thereof. A disorder can be a disease, a condition, a genotype, a phenotype, or any state associated with an adverse effect. In some embodiments, treating a disorder can comprise preventing, slowing progression of, reversing, or alleviating symptoms of the disorder. A method of treating a disorder can comprise delivering an engineered polynucleotide encoding an engineered guide RNA to a cell of a subject in need thereof and expressing the engineered guide RNA in the cell. In some embodiments, an engineered guide RNA of the present disclosure can be used to treat a genetic disorder (e.g., a Tauopathy such as AD, FTD, Parkinson's disease). In some embodiments, an engineered guide RNA of the present disclosure can be used to treat a condition associated with one or more mutations.

The present disclosure provides for compositions of expression cassettes encoding engineered payloads (e.g., engineered guide RNAs) and methods of use thereof, such as methods of treatment. In some embodiments, the expression cassettes of the present disclosure encode guide RNAs targeting a coding sequence of an RNA (e.g., e.g., an RNA encoding α-synuclein, PMP22, DUX4, LRRK2, tau, progranulin, ABCA4, amyloid precursor protein, or alpha-1 antitrypsin). In some embodiments, the engineered polynucleotides of the present disclosure encode guide RNAs targeting a non-coding sequence of an RNA (e.g., a polyA sequence). In some embodiments, the present disclosure provides compositions of one or more than one engineered polynucleotide encoding more than one engineered guide RNAs targeting the TIS, the polyA sequence, or any other part of a coding sequence or non-coding sequence. The engineered guide RNAs disclosed herein facilitate ADAR-mediated RNA editing of adenosines in the TIS, the polyA sequence, any part of a coding sequence of an RNA, any part of a non-coding sequence of an RNA, or any combination thereof.

Examples of target genes that may be targeted by engineered RNA payloads encoded by the expression cassettes of the present disclosure are provided in TABLE 10. The target gene may be a wild type gene, or the target gene may be a mutated gene. Targeting the gene using an engineered RNA payload may treat a condition associated with the target gene.

TABLE 10
Exemplary Gene Targets and Associated Conditions

| Target Gene (Protein) | Associated Conditions |
|---|---|
| SNCA (α-synuclein) | Synucleinopathies, Parkinson's disease, Lewy |
| | body dementia, multiple system atrophy |
| PMP22 (peripheral myelin protein 22) | Charcot-Marie-Tooth disease, Hereditary |
| | neuropathy with liability to pressure palsies, Yuan- |
| | Harel-Lupski syndrome |
| MAPT (Tau) | Tauopathies, Alzheimer's disease frontotemporal |
| | dementia, Parkinson's disease, progressive |
| | supranuclear palsy, corticobasal degeneration, |
| | chronic traumatic encephalopathy, autism, |
| | traumatic brain injury, Dravet syndrome |
| LRRK2 (leucine rich repeat kinase 2) | Parkinson's disease, Crohn's disease |
| DUX4 (double homeobox 4) | Muscular dystrophy, B-cell leukemia |
| CMT1A (duplication of PMP22 | Charcot-Marie-Tooth disease, Dejerine-Sottas |
| associated with Charcot-Marie-Tooth | disease, hereditary neuropathy with liability to |
| disease type 1A) | pressure palsy |
| GRN (progranulin) | Frontotemporal dementia |
| ABCA4 (ATP-binding cassette sub- | Stargardt disease |
| family A member 4) | |
| APP (amyloid precursor protein) | Alzheimer's disease |
| SERPINA1 (alpha-1 antitrypsin) | Alpha-1 antitrypsin deficiency |
| HEXA (hexosaminidase A) | Tay-Sachs disease |
| CFTR (cystic fibrosis transmembrane | Cystic fibrosis |
| conductance regulator) | |
| LIPA (lipase A) | Liposomal acid lipase deficiency |
| GBA (glucosylceramidase beta) | Gaucher disease, Parkinson's disease |
| PINK1 (PTEN-induced kinase 1) | Parkinson's disease |
| MECP2 (methyl CpG binding protein 2) | Rett syndrome |

The expression cassettes of the present disclosure may express payloads to target, modify, and/or express any sequence of interest. Select targets of interest that may be targeted by the payloads described herein for treatment of an associated condition are discussed below by way of example.

### MAPT

The present disclosure provides for expression cassettes encoding engineered guide RNAs that facilitate RNA editing MAPT to knockdown expression of Tau protein. Tau pathology can be a key driver of a broad spectrum of neurodegenerative diseases, collectively known as Tauopathies. For example, diseases where Tau can play a primary role include, but are not limited to, Alzheimer's disease (AD), frontotemporal dementia (FTD), Parkinson's disease, progressive supranuclear palsy (PSP), corticobasal degeneration (CBD), and chronic traumatic encephalopathy. Tauopathies are characterized by the intracellular accumulation of neurofibrillary tangles (NFTs) composed of aggregated, misfolded Tau (MAPT gene). Thus, engineered guide RNAs of the present disclosure targeting MAPT RNA for ADAR-mediated editing to knockdown Tau protein can be capable of preventing or ameliorating disease progression in a number of diseases, including, but not limited to, AD, FTD, autism, traumatic brain injury, Parkinson's disease, and Dravet syndrome.

Thus, the engineered guide RNAs of the present disclosure can target MAPT for RNA editing, thereby, driving a reduction in Tau protein expression. In some embodiments, Tau protein expression is reduced in human neurons. In some embodiments, the present disclosure provides compositions of engineered guide RNAs that target MAPT and facilitated ADAR-mediated RNA editing of MAPT to reduce pathogenic levels of Tau by targeting key adenosines for deamination that are present in the translational initiation sites (TISs). In some embodiments, the engineered guide RNAs of the present disclosure target a coding sequence in MAPT. For example, the coding sequence can be a translation initiation site (TIS) (AUG) of MAPT, and the engineered guide RNA can facilitate ADAR-mediated RNA editing of AUG to GUG. Engineered guide RNAs of the present disclosure can target one or more of the TISs in MAPT to reduce or completely inhibit Tau protein expression.

For example, in some embodiments, an engineered guide RNA targets the AUG at the 18^{th} nucleotide in Exon 1 (c.1, Nm_005910.5; GRCh37/Hg19; also referred to as “c.1” for coding nucleotide 1), referred to as the conventional TIS. In some embodiments, an engineered guide RNA targets the AUG at the 48^{th} nucleotide in Exon 1 (c.31). In some embodiments, an engineered guide RNA targets the AUG at the 6^{th} nucleotide in Exon 5 (c.379). With reference to the 2N4R Tau isoform containing 441 amino acids (Np_005901; GRCh37/Hg19), these three TISs correspond to methionines (Met) 1, 11 and 127, respectively. In some embodiments, an engineered guide RNA targets the AUG at the 108^{th} nucleotide in Exon 1 (c.91). In some embodiments, one or more than one engineered guide RNAs of the present disclosure target any one or any combination of said four TISs. For example, a single engineered guide RNA of the present disclosure can be designed to target more than one of the above four TISs. In some embodiments, more than one engineered guide RNAs are designed to each independently target more than one of the above four TISs. In some embodiments, engineered guide RNAs of the present disclosure can target any one or any combination of the TISs in Exon 1 (c.1, c.31, and c.91). Targeting these sites in MAPT facilitate edits that result in inhibition of translation and a reduction in expression of the Tau protein. In some embodiments, the ratio of 3R to 4R isoforms of Tau can be measured by protein analysis (e.g., using an ELISA or flow cytometry) to evaluate the effect of RNA editing, with a 1 to 1 ratio representing the ratio in healthy adult brain. In some embodiments, any of the engineered guide RNAs disclosed herein are packaged in an AAV vector and are virally delivered.

In some embodiments, the engineered guide RNAs target a non-coding sequence in MAPT. The non-coding sequence can be a polyA signal sequence and the engineered guide RNA can facilitate ADAR-mediated RNA editing of one or more adenosines in the polyA signal sequence of MAPT. In some embodiments, engineered guide RNAs of the present disclosure can be multiplexed to target more than one polyA signal sequences in MAPT. In some embodiments, engineered guide RNAs of the present disclosure can be multiplexed to target the TIS and one or more polyA signal sequences in MAPT. In some embodiments, engineered guide RNAs can be multiplexed to target a non-coding sequence and a coding sequence in MAPT. The engineered guide RNAs of the present disclosure facilitated ADAR-mediated RNA editing of MAPT, thereby, effecting protein knockdown.

In some embodiments, the engineered guide RNAs of the present disclosure facilitated ADAR-mediated RNA editing of from 1 to 100% of a target adenosine. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of MAPT, which results in knockdown of protein levels. The knockdown in protein levels is quantitated as a reduction in expression of the Tau protein. The engineered guide RNAs of the present disclosure can facilitate from 1% to 100% Tau protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% Tau protein knockdown. In some embodiments, the engineered guide RNAs of the present disclosure facilitate from 30% to 60% Tau protein knockdown. Tau protein knockdown can be measured by an assay comparing a sample or subject treated with the engineered guide RNA to a control sample or subject not treated with the engineered guide RNA.

### α-Synuclein

The alpha-synuclein gene is made up of 5 exons and encodes a 140 amino-acid protein with a predicted molecular mass of ˜14.5 kDa. The encoded product is an intrinsically disordered protein with unknown functions. Usually, Alpha-synuclein is a monomer. Under certain stress conditions or other unknown causes, α-synuclein self-aggregates into oligomers. Lewy-related pathology (LRP), primarily comprised of Alpha-synuclein in more than 50% of autopsy-confirmed Alzheimer's disease patients' brains. While the molecular mechanism of how Alpha-synuclein affects the development of Alzheimer's disease is unclear, experimental evidence has shown that Alpha-synuclein interacts with Tau-p and may seed the intracellular aggregation of Tau-p. Moreover, Alpha-synuclein could regulate the activity of GSK3β, which can mediate Tau-hyperphosphorylation. Alpha-synuclein can also self-assemble into pathogenic aggregates (Lewy bodies). Both Tau and α-synuclein can be released into the extracellular space and spread to other cells. Vascular abnormalities impair the supply of nutrients and removal of metabolic byproducts, cause microinfarcts, and promote the activation of glial cells. Therefore, a multiplex strategy to substantially reduce Tau formation, alpha-synuclein formation, or a combination thereof can be important in effectively treating neurodegenerative diseases.

The domain structure of Alpha-synuclein comprises an N-terminal A2 lipid-binding alpha-helix domain, a non-amyloid p component (NAC) domain, and a C-terminal acidic domain. Molecularly, Alpha-synuclein is suggested to play a role in neuronal transmission and DNA repair. In some cases, a region of Alpha-synuclein can be targeted utilizing compositions provided herein. In some cases, a region of the Alpha-synuclein mRNA can be targeted with the engineered polynucleotides disclosed herein for knockdown. In some cases, a region of the exon or intron of the Alpha-synuclein mRNA can be targeted. In some embodiments, a region of the non-coding sequence of the Alpha-synuclein mRNA, such as the 5′UTR and 3′UTR, can be targeted. In other cases, a region of the coding sequence of the Alpha-synuclein mRNA can be targeted. Suitable regions include but are not limited to a N-terminal A2 lipid-binding alpha-helix domain, a non-amyloid p component (NAC) domain, or a C-terminal acidic domain.

In some aspects, an alpha-synuclein mRNA sequence is targeted. In some cases, any one of the 3,177 residues of the sequence may be targeted utilizing the compositions and method provided herein. In some cases, a target residue may be located among residues 1 to 100, from 99 to 200, from 199 to 300, from 299 to 400, from 399 to 500, from 499 to 600, from 599 to 700, from 699 to 800, from 799 to 900, from 899 to 1000, from 999 to 1100, from 1099 to 1200, from 1199 to 1300, from 1299 to 1400, from 1399 to 1500, from 1499 to 1600, from 1599 to 1700, from 1699 to 1800, from 1799 to 1900, from 1899 to 2000, from 1999 to 2100, from 2099 to 2200, from 2199 to 2300, from 2299 to 2400, from 2399 to 2500, from 2499 to 2600, from 2599 to 2700, from 2699 to 2800, from 2799 to 2900, from 2899 to 3000, from 2999 to 3100, from 3099 to 3177, or any combination thereof.

In some embodiments, the present disclosure provides expression cassettes encoding engineered guide RNAs that target SNCA. The engineered guide RNAs may target SNCA to modify or alter expression of SNCA. In some embodiments, targeting SNCA with the engineered guide RNAs of the present disclosure may treat a disease associated with SNCA, such as synucleinopathies, Parkinson's disease, Lewy body dementia, or multiple system atrophy. In some embodiments, the engineered guide RNAs may facilitate ADAR-mediated RNA editing of SNCA to correct G to A mutations by targeting adenosines for deamination. The engineered guide RNAs of the present disclosure may target a coding sequence in SNCA. For example, the coding sequence can be a translation initiation site (TIS) (AUG) of AUG, and the engineered guide RNA can facilitate ADAR-mediated RNA editing of AUG to GUG. Editing of the TIS may affect protein knockdown of SNCA. In another example, the guide RNA can facilitate ADAR-mediated correction of missense mutations in the coding sequence. Correcting a missense mutation may increase expression of functional SNCA protein. In another example, the guide RNA can facilitate ADAR-mediated correction of nonsense mutations in the coding sequence. Correcting a nonsense mutation may increase expression of SNCA protein.

In some embodiments, the engineered guide RNAs target a non-coding sequence in SNCA. The non-coding sequence can be a polyA signal sequence and the engineered guide RNA can facilitate ADAR-mediated RNA editing of one or more adenosines in the polyA signal sequence of SNCA. In some embodiments, engineered guide RNAs of the present disclosure can be multiplexed to target more than one polyA signal sequences in SNCA. In some embodiments, engineered guide RNAs of the present disclosure can be multiplexed to target the TIS and one or more polyA signal sequences in SNCA. In some embodiments, engineered guide RNAs can be multiplexed to target a non-coding sequence and a coding sequence in SNCA. The engineered guide RNAs of the present disclosure facilitated ADAR-mediated RNA editing of SNCA, thereby, affecting protein knockdown.

In some embodiments, the engineered guide RNAs of the present disclosure facilitated ADAR-mediated RNA editing of from 1 to 100% of a target adenosine in SNCA. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of SNCA, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of SNCA, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### PMP22

Peripheral myelin protein 22, encoded by PMP22, is involved in myelinating Schwann cells of the peripheral nervous system. Duplication or deletion of PMP22, and corresponding alteration of gene expression levels, is associated with a variety of diseases, including Charcot-Marie-Tooth type 1A (CMT1A), Dejerine-Sottas disease, and Hereditary Neuropathy with Liability to Pressure Palsy (HNPP). Described herein are methods of editing or modifying expression of PMP22 using an expression cassette encoding an engineered RNA payload to treat a disease (e.g., Charcot-Marie-Tooth disease, Dejerine-Sottas disease, or hereditary neuropathy).

In some embodiments, the present disclosure provides expression cassettes encoding engineered guide RNAs that target PMP22. The engineered guide RNAs may target PMP22 to modify or alter expression of PMP22. In some embodiments, targeting PMP22 with the engineered guide RNAs of the present disclosure may treat a disease associated with PMP22, such as Charcot-Marie-Tooth disease, Dejerine-Sottas disease, or hereditary neuropathy. In some embodiments, the engineered guide RNAs may facilitate ADAR-mediated RNA editing of PMP22 to correct G to A mutations by targeting adenosines for deamination. The engineered guide RNAs of the present disclosure may target a coding sequence in PMP22. For example, the coding sequence can be a translation initiation site (TIS) (AUG) of AUG, and the engineered guide RNA can facilitate ADAR-mediated RNA editing of AUG to GUG. Editing of the TIS may affect protein knockdown of PMP22. In another example, the guide RNA can facilitate ADAR-mediated correction of missense mutations in the coding sequence. Correcting a missense mutation may increase expression of functional PMP22 protein. In another example, the guide RNA can facilitate ADAR-mediated correction of nonsense mutations in the coding sequence. Correcting a nonsense mutation may increase expression of PMP22 protein.

In some embodiments, the engineered guide RNAs target a non-coding sequence in PMP22. The non-coding sequence can be a polyA signal sequence and the engineered guide RNA can facilitate ADAR-mediated RNA editing of one or more adenosines in the polyA signal sequence of PMP22. In some embodiments, engineered guide RNAs of the present disclosure can be multiplexed to target more than one polyA signal sequences in PMP22. In some embodiments, engineered guide RNAs of the present disclosure can be multiplexed to target the TIS and one or more polyA signal sequences in PMP22. In some embodiments, engineered guide RNAs can be multiplexed to target a non-coding sequence and a coding sequence in PMP22. The engineered guide RNAs of the present disclosure facilitated ADAR-mediated RNA editing of PMP22, thereby, affecting protein knockdown.

In some embodiments, the engineered guide RNAs of the present disclosure facilitated ADAR-mediated RNA editing of from 1 to 100% of a target adenosine in PMP22. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of PMP22, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of PMP22, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### LRRK2

Leucine-rich repeat kinase 2 (LRRK2) has been associated with familial and sporadic cases of Parkinson's Disease and immune-related disorders like Crohn's disease. Its aliases include LRRK2, AURA17, DARDARIN, PARK8, RIPK7, ROCO2, or leucine-rich repeat kinase 2. The LRRK2 gene is made up of 51 exons and encodes a 2527 amino acid protein with a predicted molecular mass of about 286 kDa. The encoded product is a multi-domain protein with kinase and GTPase activities. LRRK2 can be found in various tissues and organs including but not limited to adrenal, appendix, bone marrow, brain, colon, duodenum, endometrium, esophagus, fat, gall bladder, heart, kidney, liver, lung, lymph node, ovary, pancreas, placenta, prostate, salivary gland, skin, small intestine, spleen, stomach, testis, thyroid, and urinary bladder. LRRK2 can be ubiquitously expressed but is generally more abundant in the brain, kidney, and lung tissue. Cellularly, LRRK2 has been found in astrocytes, endothelial cells, microglia, neurons, and peripheral immune cells.

Over 100 mutations have been identified in LRRK2; six of them—G2019S, R1441C/G/H, Y1699C, and I2020T have been shown to cause Parkinson's Disease through segregation analysis. G2019S and R1441C are the most common disease-causing mutations in inherited cases. In sporadic cases, these mutations have shown age-dependent penetrance: The percentage of individuals carrying the G2019S mutation that develops the disease jumps from 17% to 85% when the age increases from 50 to 70 years old. In some cases, mutation carrying individuals never develop the disease.

At its catalytic core, LRRK2 contains the Ras of complex proteins (Roc), C-terminal of ROC (COR), and kinase domains. Multiple protein-protein interaction domains flank this core: an armadillo repeats (ARM) region, an ankyrin repeat (ANK) region, a leucine-rich repeat (LRR) domain are found in the N-terminus joined by a C-terminal WD40 domain. The G2019S mutation is located within the kinase domain. It has been shown to increase the kinase activity; for R1441C/G/H and Y1699C, these mutations can decrease the GTPase activity of the Roc domain. Genome-wide association study has found that common variations in LRRK2 increase the risk of developing sporadic Parkinson's Disease. While some of these variations are nonconservative mutations that affect the protein's binding or catalytic activities, others modulate its expression. These results suggest that specific alleles or haplotypes can regulate LRRK2 expression.

Pro-inflammatory signals upregulate LRRK2 expression in various immune cell types, suggesting that LRRK2 is a critical regulator in the immune response. Studies have found that both systemic and central nervous system (CNS) inflammation are involved in Parkinson's Disease's symptoms. Moreover, LRRK2 mutations associated with Parkinson's Disease modulate its expression levels in response to inflammatory stimuli. Many mutations in LRRK2 are associated with immune-related disorders such as inflammatory bowel disease such as Crohn's Disease. For example, both G2019S and N2081D increase LRRK2's kinase activity and are over-represented in Crohn's Disease patients in specific populations. Because of its critical role in these disorders, LRRK2 is an important therapeutic target for Parkinson's Disease and Crohn's Disease. In particular, many mutations, such as point mutations including G2019S, play roles in developing these diseases, making LRRK2 an attractive for therapeutic strategy such as RNA editing.

In some embodiments, the present disclosure provides expression cassettes encoding guide RNAs that are capable of facilitating RNA editing of LRRK2. In some embodiments, a guide RNA of the present disclosure can target the following mutations in LRRK2: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, or Q2490NfsX3. Said guide RNAs targeting a site in LRRK2 can be encoded by an engineered polynucleotide construct of the present disclosure.

In some examples, hybridization of a latent guide RNA targeting LRRK2 to a target LRRK2 mRNA produces a guide-target RNA scaffold that comprises a structural features selected from the group consisting of: (i) one or more X1/X2 bulges, wherein Xi is the number of nucleotides of the target RNA in the bulge and X2 is the number of nucleotides of the engineered guide RNA in the bulge, and wherein the one or more bulges is a 0/1 asymmetric bulge, a 2/2 symmetric bulge, a 3/3 symmetric bulge, or a 4/4 symmetric bulge; (ii) one or more X1/X2 internal loops, wherein Xi is the number of nucleotides of the target RNA in the internal loop and X2 is the number of nucleotides of the engineered guide RNA in the internal loop, and wherein the one or more internal loops is a 5/0 asymmetric internal loop, a 5/4 asymmetric internal loop, a 5/5 symmetric internal loop, a 6/6 symmetric internal loop, a 7/7 symmetric internal loop, or a 10/10 symmetric internal loop; (iii) one or more mismatches, wherein the one or more mismatches is an A/C mismatch, an A/G mismatch, a C/U mismatch, a G/A mismatch, or a C/C mismatch, (iv) a G/U wobble base pair or a U/G wobble base pair, and (v) any combination thereof. Said engineered guide RNAs can be delivered via viral vector (e.g., encoded for and delivered via AAV) as disclosed herein and can be administered via any route of administration disclosed herein to a subject in need thereof. The subject can be human and may be at risk of developing or has developed a disease or condition associated with mutations in LRRK2 (e.g., diseases of the central nervous system (CNS) or gastrointestinal (GI) tract). For example, such diseases of conditions can include Crohn's disease or Parkinson's disease. Such CNS or GI tract diseases (e.g., Crohn's disease or Parkinson's disease) can be at least partially caused by a mutation of LRRK2, for which an engineered guide RNA described herein can facilitate editing in, thus correcting the mutation in LRRK2 and reducing the incidence of the CNS or GI tract disease in the subject. Thus, the guide RNAs of the present disclosure can be used in a method of treatment of diseases such as Crohn's disease or Parkinson's disease.

In some embodiments, the present disclosure provides expression cassettes encoding engineered guide RNAs that target LRRK2. The engineered guide RNAs may target LRRK2 to modify or alter expression of LRRK2. In some embodiments, targeting LRRK2 with the engineered guide RNAs of the present disclosure may treat a disease associated with LRRK2, such as Parkinson's disease or Crohn's disease. In some embodiments, the engineered guide RNAs may facilitate ADAR-mediated RNA editing of LRRK2 to correct G to A mutations by targeting adenosines for deamination. The engineered guide RNAs of the present disclosure may target a coding sequence in LRRK2. For example, the coding sequence can be a translation initiation site (TIS) (AUG) of AUG, and the engineered guide RNA can facilitate ADAR-mediated RNA editing of AUG to GUG. Editing of the TIS may affect protein knockdown of LRRK2. In another example, the guide RNA can facilitate ADAR-mediated correction of missense mutations in the coding sequence. Correcting a missense mutation may increase expression of functional LRRK2 protein. In another example, the guide RNA can facilitate ADAR-mediated correction of nonsense mutations in the coding sequence. Correcting a nonsense mutation may increase expression of LRRK2 protein.

In some embodiments, the engineered guide RNAs target a non-coding sequence in LRRK2. The non-coding sequence can be a polyA signal sequence and the engineered guide RNA can facilitate ADAR-mediated RNA editing of one or more adenosines in the polyA signal sequence of LRRK2. In some embodiments, engineered guide RNAs of the present disclosure can be multiplexed to target more than one polyA signal sequences in LRRK2. In some embodiments, engineered guide RNAs of the present disclosure can be multiplexed to target the TIS and one or more polyA signal sequences in LRRK2. In some embodiments, engineered guide RNAs can be multiplexed to target a non-coding sequence and a coding sequence in LRRK2. The engineered guide RNAs of the present disclosure facilitated ADAR-mediated RNA editing of LRRK2, thereby, affecting protein knockdown.

In some embodiments, the engineered guide RNAs of the present disclosure facilitated ADAR-mediated RNA editing of from 1 to 100% of a target adenosine in LRRK2. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of LRRK2, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of LRRK2, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### DUX4

Double homeobox, 4 (DUX4) functions as a transcriptional activator of a variety of genes, including PITX1, and regulates expression of small RNAs in muscle cells. In some embodiments, overexpression of DUX4 can cause B-cell leukemia. Described herein are methods of editing or modifying expression of DUX4 using an expression cassette encoding an engineered RNA payload to treat a disease (e.g., B-cell leukemia or facioscapulohumeral muscular dystrophy).

In some embodiments, the present disclosure provides expression cassettes encoding engineered guide RNAs that target DUX4. The engineered guide RNAs may target DUX4 to modify or alter expression of DUX4. In some embodiments, targeting DUX4 with the engineered guide RNAs of the present disclosure may treat a disease associated with DUX4, such as B-cell leukemia or facioscapulohumeral muscular dystrophy. In some embodiments, the engineered guide RNAs may facilitate ADAR-mediated RNA editing of DUX4 to correct G to A mutations by targeting adenosines for deamination. The engineered guide RNAs of the present disclosure may target a coding sequence in DUX4. For example, the coding sequence can be a translation initiation site (TIS) (AUG) of AUG, and the engineered guide RNA can facilitate ADAR-mediated RNA editing of AUG to GUG. Editing of the TIS may affect protein knockdown of DUX4. In another example, the guide RNA can facilitate ADAR-mediated correction of missense mutations in the coding sequence. Correcting a missense mutation may increase expression of functional DUX4 protein. In another example, the guide RNA can facilitate ADAR-mediated correction of nonsense mutations in the coding sequence. Correcting a nonsense mutation may increase expression of DUX4 protein.

In some embodiments, the engineered guide RNAs target a non-coding sequence in DUX4. The non-coding sequence can be a polyA signal sequence and the engineered guide RNA can facilitate ADAR-mediated RNA editing of one or more adenosines in the polyA signal sequence of DUX4. In some embodiments, engineered guide RNAs of the present disclosure can be multiplexed to target more than one polyA signal sequences in DUX4. In some embodiments, engineered guide RNAs of the present disclosure can be multiplexed to target the TIS and one or more polyA signal sequences in DUX4. In some embodiments, engineered guide RNAs can be multiplexed to target a non-coding sequence and a coding sequence in DUX4. The engineered guide RNAs of the present disclosure facilitated ADAR-mediated RNA editing of DUX4, thereby, affecting protein knockdown.

In some embodiments, the engineered guide RNAs of the present disclosure facilitated ADAR-mediated RNA editing of from 1 to 100% of a target adenosine in DUX4. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of DUX4, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of DUX4, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### Progranulin

Progranulin, encoded by GRN, is a precursor protein cleaved to form granulin. GRN is expressed in peripheral and central nervous system tissues and is upregulated in microglia following injury. Both granulin and progranulin are implicated in a wide variety of functions, including development, inflammation, cell proliferation, and protein homeostasis. Mutations in GRN are implicated in frontotemporal dementia. Described herein are methods of editing or modifying expression of GRN using an expression cassette encoding an engineered RNA payload to treat a disease (e.g., frontotemporal dementia).

In some embodiments, the present disclosure provides expression cassettes encoding engineered guide RNAs that target GRN. The engineered guide RNAs may target GRN to modify or alter expression of GRN. In some embodiments, targeting GRN with the engineered guide RNAs of the present disclosure may treat a disease associated with GRN, such as frontotemporal dementia. In some embodiments, the engineered guide RNAs may facilitate ADAR-mediated RNA editing of GRN to correct G to A mutations by targeting adenosines for deamination. The engineered guide RNAs of the present disclosure may target a coding sequence in GRN. For example, the coding sequence can be a translation initiation site (TIS) (AUG) of AUG, and the engineered guide RNA can facilitate ADAR-mediated RNA editing of AUG to GUG. Editing of the TIS may affect protein knockdown of GRN. In another example, the guide RNA can facilitate ADAR-mediated correction of missense mutations in the coding sequence. Correcting a missense mutation may increase expression of functional GRN protein. In another example, the guide RNA can facilitate ADAR-mediated correction of nonsense mutations in the coding sequence. Correcting a nonsense mutation may increase expression of GRN protein.

In some embodiments, the engineered guide RNAs target a non-coding sequence in GRN. The non-coding sequence can be a polyA signal sequence and the engineered guide RNA can facilitate ADAR-mediated RNA editing of one or more adenosines in the polyA signal sequence of GRN. In some embodiments, engineered guide RNAs of the present disclosure can be multiplexed to target more than one polyA signal sequences in GRN. In some embodiments, engineered guide RNAs of the present disclosure can be multiplexed to target the TIS and one or more polyA signal sequences in GRN. In some embodiments, engineered guide RNAs can be multiplexed to target a non-coding sequence and a coding sequence in GRN. The engineered guide RNAs of the present disclosure facilitated ADAR-mediated RNA editing of GRN, thereby, affecting protein knockdown.

In some embodiments, the engineered guide RNAs of the present disclosure facilitated ADAR-mediated RNA editing of from 1 to 100% of a target adenosine in GRN. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of GRN, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of GRN, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### ABCA4

In some embodiments, the present disclosure provides expression cassettes encoding guide RNAs that are capable of facilitating RNA editing of ATP binding cassette subfamily A member 4 (ABCA4). In some examples, the disease or condition can be associated with a mutation in an ABCA4 gene. In some examples, the disease or condition can be Stargardt macular degeneration. In some examples, the Stargardt macular degeneration can be caused, at least in part, by a mutation in an ABCA4 gene. In some examples, the mutation comprises a substitution of a G with an A at nucleotide position 5882 in a wildtype ABCA4 gene. In some examples, the mutation comprises a G with an A at nucleotide position 5714 in a wildtype ABCA4 gene. In some examples, the mutation comprises a substitution of a G with an A at nucleotide position 6320 in a wildtype ABCA4 gene. In some examples, the double stranded substrate mimics one or more structural features of the naturally occurring ADAR substrate and comprises a target mRNA molecule encoded by the ABCA4 gene and an engineered guide that can be complementary, at least in part, to a portion of the target mRNA molecule.

In some examples, hybridization of a latent guide RNA targeting ABCA4 to a target ABCA4 mRNA produces a guide-target RNA scaffold that comprises a structural features selected from the group consisting of: (i) one or more X1/X2 bulges, wherein Xi is the number of nucleotides of the target RNA in the bulge and X2 is the number of nucleotides of the engineered guide RNA in the bulge, and wherein the one or more bulges is a 2/1 asymmetric bulge, a 1/0 asymmetric bulge, a 2/2 symmetric bulge, a 3/3 symmetric bulge, or a 4/4 symmetric bulge; (ii) an X1/X2 internal loop, wherein Xi is the number of nucleotides of the target RNA in the internal loop and X2 is the number of nucleotides of the engineered guide RNA in the internal loop, and wherein the internal loop is a 5/5 symmetric loop (iii) one or more mismatches, wherein the one or more mismatches is a G/G mismatch, an A/C mismatch, or a G/A mismatch, (iv) a G/U wobble base pair or a U/G wobble base pair, and (v) any combination thereof. In some embodiments, the guide-target RNA scaffold comprises a 2/1 asymmetric bulge, a 1/0 asymmetric bulge, a G/G mismatch, an A/C mismatch, and a 3/3 symmetric bulge. In some instances, the engineered latent guide RNA targeting ABCA4 comprises a G/G mismatch, a U/U mismatch, and a G/G mismatch. Said engineered guide RNAs can be delivered via viral vector (e.g., encoded for and delivered via AAV) as disclosed herein and can be administered via any route of administration disclosed herein to a subject in need thereof. The subject can be human and may be at risk of developing or has developed Stargardt macular degeneration (or Stargardt's disease). Such Stargardt macular degeneration can be at least partially caused by a mutation of ABCA4, for which an engineered guide RNA described herein can facilitate editing in, thus correcting the mutation in ABCA4 and reducing the incidence of Stargardt macular degeneration in the subject. Thus, the guide RNAs of the present disclosure can be used in a method of treatment of Stargardt macular degeneration.

In some embodiments, the present disclosure provides expression cassettes encoding engineered guide RNAs that target ABCA4. The engineered guide RNAs may target ABCA4 to modify or alter expression of ABCA4. In some embodiments, targeting ABCA4 with the engineered guide RNAs of the present disclosure may treat a disease associated with ABCA4, such as Stargardt disease. In some embodiments, the engineered guide RNAs may facilitate ADAR-mediated RNA editing of ABCA4 to correct G to A mutations by targeting adenosines for deamination. The engineered guide RNAs of the present disclosure may target a coding sequence in ABCA4. For example, the coding sequence can be a translation initiation site (TIS) (AUG) of AUG, and the engineered guide RNA can facilitate ADAR-mediated RNA editing of AUG to GUG. Editing of the TIS may affect protein knockdown of ABCA4. In another example, the guide RNA can facilitate ADAR-mediated correction of missense mutations in the coding sequence. Correcting a missense mutation may increase expression of functional ABCA4 protein. In another example, the guide RNA can facilitate ADAR-mediated correction of nonsense mutations in the coding sequence. Correcting a nonsense mutation may increase expression of ABCA4 protein.

In some embodiments, the engineered guide RNAs target a non-coding sequence in ABCA4. The non-coding sequence can be a polyA signal sequence and the engineered guide RNA can facilitate ADAR-mediated RNA editing of one or more adenosines in the polyA signal sequence of ABCA4. In some embodiments, engineered guide RNAs of the present disclosure can be multiplexed to target more than one polyA signal sequences in ABCA4. In some embodiments, engineered guide RNAs of the present disclosure can be multiplexed to target the TIS and one or more polyA signal sequences in ABCA4. In some embodiments, engineered guide RNAs can be multiplexed to target a non-coding sequence and a coding sequence in ABCA4. The engineered guide RNAs of the present disclosure facilitated ADAR-mediated RNA editing of ABCA4, thereby, affecting protein knockdown.

In some embodiments, the engineered guide RNAs of the present disclosure facilitated ADAR-mediated RNA editing of from 1 to 100% of a target adenosine in ABCA4. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of ABCA4, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of ABCA4, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### Amyloid Precursor Protein

An expression cassette of the present disclosure can be used to express an engineered polynucleotide payload sequence targeting an amyloid precursor protein (APP). In some embodiments, the engineered polynucleotides can target a secretase enzyme cleavage site in APP and edit said cleavage site in order to modulate processing and cleavage of APP by secretase enzymes (e.g., a beta secretase such as BACE1, cathepsin B or Meprin beta). In some embodiments, the engineered polynucleotides can modulate the expression of APP. In some cases, the engineered polynucleotides can modulate the transcription or post-transcriptional regulation of the APP mRNA or pre-mRNA. In other cases, the engineered polynucleotides can correct aberrant expression of splice variants generated by a mutation in APP. In some cases, the engineered polynucleotides can modulate the gene or protein translation of APP. In some embodiments, the engineered polynucleotides can decrease, down-regulate, or knock down the expression of APP by decreasing the abundance of the APP transcript. In some instances, the engineered polynucleotides can decrease or down-regulate the processing, splicing, turnover or stability of the APP transcript; or the accessibility of the APP transcript by translational machinery such as ribosome. In some cases, an engineered polynucleotide can facilitate a knockdown of APP. A knockdown can reduce the expression of APP. In some cases, a knockdown can be accompanied by editing of the APP mRNA or pre-mRNA. In some cases, a knockdown can occur with substantially little to no editing of the APP mRNA or pre-mRNA. In some instances, a knockdown can occur by targeting an untranslated region of the APP mRNA or pre-mRNA, such as a 3′ UTR, a 5′ UTR or both. In some cases, a knockdown can occur by targeting a coding region of the APP mRNA or pre-mRNA.

Compositions described herein can edit the cleavage site in APP, so that P/y secretases exhibit reduced cleavage of APP or can no longer cut APP, and therefore reduced levels of Abeta 40/Abeta 42 or no Abetas can be produced. Compositions consistent with the present disclosure may combine compositions for target APP cleavage site editing with compositions for Tau (e.g., a microtubule-associated protein Tau (MAPT) encoded from a MAPT gene) knockdown or compositions for Alpha-synuclein (SNCA) knockdown and can have synergistic effects to prevent and/or cure a neurodegenerative disease. The compositions and methods disclosed herein can yield results in editing and/or knockdown of targets without any of the resulting issues seen in small molecule or antibody therapy. Compositions can knockdown APP (instead of target cleavage site editing). Editing at the target cleavage site in APP and knockdown can be deployed singly or in combination.

In some cases, a targeting sequence of an engineered polynucleotide provided herein can at least partially hybridize to a region of a target RNA. A region of a target RNA can comprise: (a) a sequence that at least partially encodes for a suitable target provided herein, (b) a sequence that is proximal to a sequence that at least partially encodes for a suitable target provided herein, (c) comprises (a) and (b). For example, a region of a target RNA can comprise (a) a sequence that at least partially encodes for an APP, (b) a sequence that is proximal to a sequence that at least partially encodes for an APP, or (c) comprises (a) and (b). Other suitable targets can be targeted with engineered polynucleotides disclosed herein. Amyloid precursor protein (APP)

Pathogenic cleavage of amyloid precursor protein (APP) can create Amyloid beta (Abeta) fragments, which has been implicated in Alzheimer's disease. The accumulation of Abeta fragments can: impair synaptic functions and related signaling pathways, change neuronal activities, trigger the release of neurotoxic mediators from glial cells, or any combination thereof. Abeta can alter kinase function, leading to Tau hyperphosphorylation.

The generation of Abeta by enzymatic cleavages of the β-amyloid precursor protein (APP) is an important player in Alzheimer's disease. The non-amyloidogenic APP processing pathway involves cleavages by alpha- and gamma-secretase. The cleavage by alpha-secretase generates a long form of secreted APP (APPs alpha) and a C-terminal fragment (alpha-CTF). Further processing of alpha-CTF by gamma-secretase generates a p3 and AICD fragment. The amyloidogenic APP processing pathway instead involves cleavages by beta- and gamma-secretase. The cleavage by beta-secretase generates a short form of secreted APP (APPs beta) and a C-terminal fragment (beta-CTF). Further processing of beta-CTF by gamma-secretase generates an Abeta and AICD fragment. The oligomerization and fibrillization of Abeta fragments lead to AD pathology. In some cases, amyloid precursor protein (APP) can be cut by a beta secretase (e.g., BACE1, cathepsin B or Meprin beta) or gamma secretase, and the fragment resulting from such cuts can be Abeta peptides of 36-43 amino acids. Certain Abeta peptide metabolites of this cleavage can be crucially involved in Alzheimer's disease pathology and progression.

In some embodiments, the present disclosure provides expression cassettes encoding engineered guide RNAs that target APP. The engineered guide RNAs may facilitate ADAR-mediated RNA editing of APP to correct G to A mutations by targeting adenosines for deamination. In some embodiments, the engineered guide RNAs of the present disclosure target a coding sequence in APP. For example, the coding sequence can be a translation initiation site (TIS) (AUG) of AUG, and the engineered guide RNA can facilitate ADAR-mediated RNA editing of AUG to GUG. Editing of the TIS may affect protein knockdown of APP. In another example, the guide RNA can facilitate ADAR-mediated correction of missense mutations in the coding sequence. Correcting a missense mutation may increase expression of functional AAP protein. In another example, the guide RNA can facilitate ADAR-mediated correction of nonsense mutations in the coding sequence. Correcting a nonsense mutation may increase expression of AAP protein.

In some embodiments, the engineered guide RNAs target a non-coding sequence in APP. The non-coding sequence can be a polyA signal sequence and the engineered guide RNA can facilitate ADAR-mediated RNA editing of one or more adenosines in the polyA signal sequence of APP. In some embodiments, engineered guide RNAs of the present disclosure can be multiplexed to target more than one polyA signal sequences in APP. In some embodiments, engineered guide RNAs of the present disclosure can be multiplexed to target the TIS and one or more polyA signal sequences in APP. In some embodiments, engineered guide RNAs can be multiplexed to target a non-coding sequence and a coding sequence in APP. The engineered guide RNAs of the present disclosure facilitated ADAR-mediated RNA editing of APP, thereby, affecting protein knockdown.

In some embodiments, the engineered guide RNAs of the present disclosure facilitated ADAR-mediated RNA editing of from 1 to 100% of a target adenosine in APP. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of APP, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of APP, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### SERPINA1

In some embodiments, the present disclosure provides expression cassettes encoding guide RNAs that are capable of facilitating RNA editing of serpin family A member 1 (SERPINA1). In some examples, the disease or condition can be an AAT deficiency or an associated lung or liver pathology (e.g., chronic obstructive pulmonary disease, cirrhosis, hepatocellular carcinoma) caused, at least in part, by a mutation in a SERPINA1 gene. In some examples, the mutation can be a substitution of a G with an A at nucleotide position 9989 within a wildtype SERPINA1 gene. In some examples, administration of the engineered guides disclosed herein restores expression of a normal AAT protein (e.g., as compared to an inactive or defective AAT protein) in a subject with an AAT deficiency. In some examples, a double stranded RNA (dsRNA) substrate (a guide-target RNA scaffold) is formed upon hybridization of an engineered guide of the present disclosure to a target RNA. In some examples, the target RNA forming the double stranded substrate comprises a portion of a mRNA or pre-mRNA molecule encoded by the SERPINA1 gene. In some examples the targeting region of the engineered guide forming the double stranded substrate is, at least in part, complementary to a portion of a mRNA or pre-mRNA molecule encoded by the SERPINA1 gene. In some examples the double stranded substrate comprises a single mismatch. In some examples, the engineered substrate additionally comprises one or two bulges. In some examples, the double stranded substrate can be formed by a target RNA comprising a mRNA or pre-mRNA encoded by the SERPINA1 gene and an engineered guide complementary to a portion of the mRNA encoded by the SERPINA1 gene, wherein the engineered substrate comprises a single mismatch. In some examples, the double stranded substrate can be formed by a target RNA comprising a mRNA or pre-mRNA encoded by the SERPINA1 gene and an engineered guide complementary to a portion of the mRNA or pre-mRNA encoded by the SERPINA1 gene, wherein the engineered substrate comprises a single mismatch, and wherein the engineered substrate comprises two additional bulges.

Guide RNAs can facilitate correction of a G to A mutation at nucleotide position 9989 of a SERPINA1 gene. In some embodiments, a guide RNA of the present disclosure can target, for example, E342K of SERPINA1. Said guide RNAs targeting a site in SERPINA1 can be encoded for by an engineered polynucleotide construct of the present disclosure.

In some embodiments, the present disclosure provides expression cassettes encoding engineered guide RNAs that target SERPINA1. The engineered guide RNAs may target SERPINA1 to modify or alter expression of SERPINA1. In some embodiments, targeting SERPINA1 with the engineered guide RNAs of the present disclosure may treat a disease associated with SERPINA1, such as alpha-1 antitrypsin deficiency. In some embodiments, the engineered guide RNAs may facilitate ADAR-mediated RNA editing of SERPINA1 to correct G to A mutations by targeting adenosines for deamination. The engineered guide RNAs of the present disclosure may target a coding sequence in SERPINA1. For example, the coding sequence can be a translation initiation site (TIS) (AUG) of AUG, and the engineered guide RNA can facilitate ADAR-mediated RNA editing of AUG to GUG. Editing of the TIS may affect protein knockdown of SERPINA1. In another example, the guide RNA can facilitate ADAR-mediated correction of missense mutations in the coding sequence. Correcting a missense mutation may increase expression of functional SERPINA1 protein. In another example, the guide RNA can facilitate ADAR-mediated correction of nonsense mutations in the coding sequence. Correcting a nonsense mutation may increase expression of SERPINA1 protein.

In some embodiments, the engineered guide RNAs target a non-coding sequence in SERPINA1. The non-coding sequence can be a polyA signal sequence and the engineered guide RNA can facilitate ADAR-mediated RNA editing of one or more adenosines in the polyA signal sequence of SERPINA1. In some embodiments, engineered guide RNAs of the present disclosure can be multiplexed to target more than one polyA signal sequences in SERPINA1. In some embodiments, engineered guide RNAs of the present disclosure can be multiplexed to target the TIS and one or more polyA signal sequences in SERPINA1. In some embodiments, engineered guide RNAs can be multiplexed to target a non-coding sequence and a coding sequence in SERPINA1. The engineered guide RNAs of the present disclosure facilitated ADAR-mediated RNA editing of SERPINA1, thereby, affecting protein knockdown.

In some embodiments, the engineered guide RNAs of the present disclosure facilitated ADAR-mediated RNA editing of from 1 to 100% of a target adenosine in SERPINA1. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the engineered guide RNAs of the present disclosure facilitate ADAR-mediated RNA editing of SERPINA1, which results in knockdown of protein levels. The knockdown in protein levels is quantitated as a reduction in expression of protein. The engineered guide RNAs of the present disclosure can facilitate from 1% to 100% protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% protein knockdown. In some embodiments, the engineered guide RNAs of the present disclosure facilitate from 30% to 60% protein knockdown. Protein knockdown can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of SERPINA1, which results in increased protein expression levels. The knockdown in protein levels is quantitated as an increase in expression of the target protein. The engineered guide RNAs of the present disclosure can facilitate from 1.1-fold to 1000-fold increased protein expression. The engineered guide RNAs of the present disclosure can facilitate from 1.1-fold to 1000-fold, from 1.5-fold to 1000-fold, from 2-fold to 1000-fold, from 5-fold to 1000-fold, from 10-fold to 1000-fold, from 20-fold to 1000-fold, from 50-fold to 1000-fold, from 100-fold to 1000-fold, from 200-fold to 1000-fold, from 500-fold to 1000-fold, from 1.1-fold to 10-fold, from 1.5-fold to 10-fold, from 2-fold to 10-fold, from 5-fold to 10-fold, from 10-fold to 100-fold, from 20-fold to 100-fold, or from 50-fold to 100-fold increased protein expression. In some embodiments, the engineered guide RNAs of the present disclosure facilitate at least 1.1-fold, at least 1.5-fold, at least 2-fold, at least 5-fold, at least 10-fold, at least 20-fold, at least 50-fold, at least 100-fold, at least 200-fold, or at least 500-fold increased expression. Increase in protein expression can be measured by an assay comparing a sample or subject treated with the engineered guide RNA to a control sample or subject not treated with the engineered guide RNA.

### Recombinant Vectors and Delivery

In some embodiments, an expression cassette (e.g., encoding a small RNA payload, such as an engineered guide RNA) of the present disclosure is introduced into a subject via a delivery vehicle. In some embodiments, the delivery vehicle is a vector. In some embodiments the vector is a plasmid, a viral vector, an expression cassette, or a transformed cell. A vector can facilitate delivery of the engineered polynucleotide into a cell to genetically modify the cell. In some examples, the vector comprises DNA, such as double stranded or single stranded DNA. In some examples, the delivery vector can be a eukaryotic vector, a prokaryotic vector (e.g., a bacterial vector or plasmid), a viral vector, or any combination thereof. In some embodiments, the vector is an expression cassette. In some embodiments, a viral vector comprises a viral capsid, an inverted terminal repeat sequence, and the engineered polynucleotide can be used to deliver the small RNA payload to a cell.

In some embodiments, a vector may comprise multiple expression cassettes of the present disclosure. An expression cassette may comprise a promoter, a payload sequence (e.g., encoding a small RNA payload, such as an engineered guide RNA), and a termination sequence. In some embodiments, a vector may comprise one or more expression cassettes. In some embodiments, a vector may comprise two or more expression cassettes. In some embodiments, a vector may comprise three or more expression cassettes. In some embodiments, a vector may comprise four or more expression cassettes. A vector comprising multiple expression cassettes may include one or more promoters, one or more payload sequences, and one or more termination sequences. In some embodiments, a vector comprising multiple expression cassettes may comprises one or more promoters (e.g., one or more of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263), one or more payload sequences, and one or more termination sequences (e.g., one or more of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289). In some embodiments, a vector comprising two or more expression cassettes may comprise two or more promoters (e.g., two or more of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263). In some embodiments, the two or more promoters may have different sequences. In some embodiments, the two or more promoters may have the same sequence. In some embodiments, a vector comprising two or more expression cassettes may comprise two or more termination sequences (e.g., two or more of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289). In some embodiments, the two or more termination sequences may have different sequences. In some embodiments, the two or more termination sequences may have the same sequence. In some embodiments, the present disclosure provides for an AAV vector comprising two expression cassettes, where a first expression cassette comprises a first promoter sequence (e.g., one or more of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263) and a first termination sequence (e.g., one or more of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289) and where the second expression cassette comprises a second promoter sequence different from the first promoter sequence (e.g., one or more of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263) and a second termination sequence different from the first termination sequence (e.g., one or more of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289). For example, a vector comprising two or more expression cassettes may have a first promoter sequence of SEQ ID NO: 17, a first termination sequence of SEQ ID NO: 1264, a second promoter sequence of SEQ ID NO: 1262, and a second termination sequence of SEQ ID NO: 1265.

In some embodiments, the viral vector can be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector can be a recombinant vector, a hybrid vector, a chimeric vector, a self-complementary vector, a single-stranded vector, or any combination thereof.

In some embodiments, the viral vector is an adenoviral vector, an adeno-associated viral vector, or a lentiviral vector. Adeno-associated virus (AAV) vectors include vectors derived from any AAV serotype, including, but not limited to AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68.

In some embodiments, a polynucleotide is introduced into a subject by non-viral vector systems. In some embodiments, cationic lipids, polymers, hydrodynamic injection and/or ultrasound may be used in delivering a polynucleotide to a subject in the absence of virus.

In some examples, the vector may be a eukaryotic vector, a prokaryotic vector (e.g., a bacterial vector) a viral vector, or any combination thereof. In some examples, the vector may be a viral vector. In some embodiments, the viral vector may be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector may be a recombinant vector, a hybrid vector, a chimeric vector, a self-complementary vector, a single-stranded vector, or any combination thereof.

In some embodiments, the viral vector may be an adeno-associated virus (AAV). In some embodiments, the AAV may be any AAV known in the art. In some embodiments, the viral vector may be of a specific serotype. In some embodiments, the viral vector may be an AAV1 serotype, AAV2 serotype, AAV3 serotype, AAV4 serotype, AAV5 serotype, AAV6 serotype, AAV7 serotype, AAV8 serotype, AAV9 serotype, AAV10 serotype, AAV 11 serotype, AAV 12 serotype, AAV13 serotype, AAV14 serotype, AAV15 serotype, AAV16 serotype, AAV-DJ serotype, AAV-DJ/8 serotype, AAV-DJ/9 serotype, AAV1/2 serotype, AAV.rh8 serotype, AAV.rh10 serotype, AAV.rh20 serotype, AAV.rh39 serotype, AAV.Rh43 serotype, AAV.Rh74 serotype, AAV.v66 serotype, AAV.Oligo001 serotype, AAV.SCH9 serotype, AAV.r3.45 serotype, AAV.RHM4-1 serotype, AAV.hu37 serotype, AAV.Anc80 serotype, AAV.Anc80L65 serotype, AAV.7m8 serotype, AAV.PhP.eB serotype, AAV.PhP.V1 serotype, AAV.PHP.B serotype, AAV.PhB.C1 serotype, AAV.PhB.C2 serotype, AAV.PhB.C3 serotype, AAV.PhB.C6 serotype, AAV.cy5 serotype, AAV2.5 serotype, AAV2tYF serotype, AAV3B serotype, AAV.LK03 serotype, AAV.HSC1 serotype, AAV.HSC2 serotype, AAV.HSC3 serotype, AAV.HSC4 serotype, AAV.HSC5 serotype, AAV.HSC6 serotype, AAV.HSC7 serotype, AAV.HSC8 serotype, AAV.HSC9 serotype, AAV.HSC10 serotype, AAV.HSC11 serotype, AAV.HSC12 serotype, AAV.HSC13 serotype, AAV.HSC14 serotype, AAV.HSC15 serotype, AAV.HSC16 serotype, AAV.HSC17 serotype, or AAVhu68 serotype, a derivative of any of these serotypes, or any combination thereof.

In some embodiments, the AAV vector may be a recombinant vector, a hybrid AAV vector, a chimeric AAV vector, a self-complementary AAV (scAAV) vector, a single-stranded AAV, or any combination thereof.

In some embodiments, the AAV vector may be a recombinant AAV (rAAV) vector. Methods of producing recombinant AAV vectors may be known in the art and generally involve, in some cases, introducing into a producer cell line: (1) DNA necessary for AAV replication and synthesis of an AAV capsid, (b) one or more helper constructs comprising the viral functions missing from the AAV vector, (c) a helper virus, and (d) the plasmid construct containing the genome of the AAV vector, e.g., ITRs, promoter and payload sequences, etc. In some examples, the viral vectors described herein may be engineered through synthetic or other suitable means by references to published sequences, such as those that may be available in the literature. For example, the genomic and protein sequences of various serotypes of AAV, as well as the sequences of the native terminal repeats (TRs), Rep proteins, and capsid subunits may be known in the art and may be found in the literature or in public databases such as GenBank or Protein Data Bank (PDB).

In some examples, methods of producing delivery vectors herein comprising packaging a polynucleotide of the present disclosure in an AAV vector. In some examples, methods of producing the delivery vectors described herein comprise, (a) introducing into a cell: (i) a polynucleotide disclosed herein; and (ii) a viral genome comprising a Replication (Rep) gene and Capsid (Cap) gene that encodes a wild type AAV capsid protein or modified version thereof; (b) expressing in the cell the wild type AAV capsid protein or modified version thereof; (c) assembling an AAV particle; and (d) packaging the polynucleotide disclosed herein in the AAV particle, thereby generating an AAV delivery vector. In some examples, any polynucleotide disclosed herein may be packaged in the AAV vector. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site, thereby enabling formation of a self-complementary AAV.

In some examples, a hybrid AAV vector may be produced by transcapsidation, e.g., packaging an inverted terminal repeat (ITR) from a first serotype into a capsid of a second serotype, wherein the first and second serotypes may be not the same. In some examples, the Rep gene and ITR from a first AAV serotype (e.g., AAV2) may be used in a capsid from a second AAV serotype (e.g., AAV5 or AAV9), wherein the first and second AAV serotypes may not be the same. As a non-limiting example, a hybrid AAV serotype comprising the AAV2 ITRs and AAV9 capsid protein may be indicated AAV2/9. In some examples, the hybrid AAV delivery vector comprises an AAV2/1, AAV2/2, AAV 2/4, AAV2/5, AAV2/8, or AAV2/9 vector.

In some examples, the AAV vector may be a chimeric AAV vector. In some examples, the chimeric AAV vector comprises an exogenous amino acid or an amino acid substitution, or capsid proteins from two or more serotypes. In some examples, a chimeric AAV vector may be genetically engineered to increase transduction efficiency, selectivity, or a combination thereof.

In some examples, the AAV vector comprises a self-complementary AAV genome. Self-complementary AAV genomes may be generally known in the art and contain both DNA strands which can anneal together to form double-stranded DNA.

In some examples, the delivery vector may be a retroviral vector. In some examples, the retroviral vector may be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector may be transfected such that the majority of sequences coding for the structural genes of the virus (e.g., gag, pol, and env) may be deleted and replaced by the gene(s) of interest.

In some examples, the delivery vehicle may be a non-viral vector. Examples of non-viral vectors may include plasmids, lipid nanoparticles, lipoplexes, polymersomes, polyplexes, dendrimers, nanoparticles, and cell-penetrating peptides. The non-viral vector may comprise a polynucleotide, such as a plasmid, encoding for a promoter (e.g., comprising a cell type- or cell state-specific response element and a switchable core promoter) and a payload sequence. In some examples, the delivery vehicle may be a plasmid. In some examples, the plasmid may be a minicircle plasmid. In some embodiments, a vector may comprise naked DNA (e.g., a naked DNA plasmid). In some embodiments, the non-viral vector comprises DNA. In some embodiments, the non-viral vector comprises RNA. In some examples, the non-viral vector comprises circular double-stranded DNA. In some examples, the non-viral vector may comprise a linear polynucleotide. In some examples, the non-viral vector comprises a polynucleotide encoding one or more genes of interest and one or more regulatory elements. In some examples, the non-viral vector comprises a bacterial backbone containing an origin of replication and an antibiotic resistance gene or other selectable marker for plasmid amplification in bacteria. In some examples, the non-viral vector contains one or more genes that provide a selective marker to induce a target cell to retain a polynucleotide (e.g., a plasmid) of the non-viral vector. In some examples, the non-viral vector may be formulated for delivery through injection by a needle carrying syringe. In some examples, the non-viral vector may be formulated for delivery via electroporation. In some examples, a polynucleotide of the non-viral vector may be engineered through synthetic or other suitable means known in the art. For example, in some cases, the genetic elements may be assembled by restriction digest of the desired genetic sequence from a donor plasmid or organism to produce ends of the DNA which may then be readily ligated to another genetic sequence.

In some embodiments, the vector containing the expression cassette is a non-viral vector system. In some embodiments, the non-viral vector system comprises cationic lipids, or polymers. For example, the non-viral vector system comprises can be a liposome or polymeric nanoparticle. In some embodiments, the small RNA payload or a non-viral vector comprising the small RNA payload is delivered to a cell by hydrodynamic injection or ultrasound.

### Pharmaceutical Compositions

Methods for treatment of diseases or disorders characterized by genetic mutations or aberrant gene expression are also encompassed by the present disclosure. Said methods include administering a therapeutically effective amount of a payload sequence as part of a recombinant polynucleotide cassette. The recombinant polynucleotide cassette of the disclosure can be formulated in pharmaceutical compositions. These compositions can comprise, in addition to one or more of the recombinant polynucleotide cassettes, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

The compositions described herein (e.g., compositions comprising an engineered guide RNA or an engineered polynucleotide) can be formulated with a pharmaceutically acceptable carrier for administration to a subject (e.g., a human or a non-human animal). A pharmaceutically acceptable carrier can include, but is not limited to, phosphate buffered saline solution, water, emulsions (e.g., an oil/water emulsion or a water/oil emulsions), glycerol, liquid polyethylene glycols, aprotic solvents such (e.g., dimethylsulfoxide, N-methylpyrrolidone, or mixtures thereof), and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. Additional examples of carriers, stabilizers, and adjuvants consistent with the compositions of the present disclosure can be found in, for example, Remington's Pharmaceutical Sciences, 21st Ed., Mack Publ. Co., Easton, Pa. (2005), incorporated herein by reference in its entirety.

In some examples, the pharmaceutical composition can be formulated in unit dose forms or multiple-dose forms. In some examples, the unit dose forms can be physically discrete units suitable for administration to human or non-human subjects (e.g., animals). In some examples, the unit dose forms can be packaged individually. In some examples, each unit dose contains a predetermined quantity of an active ingredient(s) that can be sufficient to produce the desired therapeutic effect in association with pharmaceutical carriers, diluents, excipients, or any combination thereof. In some examples, the unit dose forms comprise ampules, syringes, or individually packaged tablets and capsules, or any combination thereof. In some instances, a unit dose form can be comprised in a disposable syringe. In some instances, unit-dosage forms can be administered in fractions or multiples thereof. In some examples, a multiple-dose form comprises a plurality of identical unit dose forms packaged in a single container, which can be administered in segregated a unit dose form. In some examples, multiple dose forms comprise vials, bottles of tablets or capsules, or bottles of pints or gallons. In some instances, a multiple-dose forms comprise the same pharmaceutically active agents. In some instances, a multiple-dose forms comprise different pharmaceutically active agents.

In some examples, the pharmaceutical composition comprises a pharmaceutically acceptable excipient. In some examples, the excipient comprises a buffering agent, a cryopreservative, a preservative, a stabilizer, a binder, a compaction agent, a lubricant, a chelator, a dispersion enhancer, a disintegration agent, a flavoring agent, a sweetener, or a coloring agent, or any combination thereof.

In some examples, an excipient comprises a buffering agent. In some examples, the buffering agent comprises sodium citrate, magnesium carbonate, magnesium bicarbonate, calcium carbonate, calcium bicarbonate, or any combination thereof. In some examples, the buffering agent comprises sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, or calcium hydroxide and other calcium salts, or any combination thereof.

In some examples, an excipient comprises a cryopreservative. In some examples, the cryopreservative comprises DMSO, glycerol, polyvinylpyrrolidone (PVP), or any combination thereof. In some examples, a cryopreservative comprises a sucrose, a trehalose, a starch, a salt of any of these, a derivative of any of these, or any combination thereof. In some examples, an excipient comprises a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. In some examples, an excipient comprises a surfactant, a sugar, an amino acid, an antioxidant, a salt, a non-ionic surfactant, a solubilizer, a triglyceride, an alcohol, or any combination thereof. In some examples, an excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. In some examples, the excipient can be an excipient described in the Handbook of Pharmaceutical Excipients, American Pharmaceutical Association (1986).

In some examples, the excipient comprises a preservative. In some examples, the preservative comprises an antioxidant, such as alpha-tocopherol and ascorbate, an antimicrobial, such as parabens, chlorobutanol, and phenol, or any combination thereof. In some examples, the antioxidant comprises EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol or N-acetyl cysteine, or any combination thereof. In some examples, the preservative comprises validamycin A, TL-3, sodium ortho vanadate, sodium fluoride, N-α-tosyl-Phe-chloromethylketone, N-α-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitors, or any combination thereof.

In some examples, the excipient comprises a binder. In some examples, the binder comprises starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, or any combination thereof.

In some examples, the binder can be a starch, for example a potato starch, corn starch, or wheat starch; a sugar such as sucrose, glucose, dextrose, lactose, or maltodextrin; a natural and/or synthetic gum; a gelatin; a cellulose derivative such as microcrystalline cellulose, hydroxypropyl cellulose, hydroxyethyl cellulose, hydroxypropyl methyl cellulose, carboxymethyl cellulose, methyl cellulose, or ethyl cellulose; polyvinylpyrrolidone (povidone); polyethylene glycol (PEG); a wax; calcium carbonate; calcium phosphate; an alcohol such as sorbitol, xylitol, mannitol, or water, or any combination thereof.

In some examples, the excipient comprises a lubricant. In some examples, the lubricant comprises magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, or light mineral oil, or any combination thereof. In some examples, the lubricant comprises metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate or talc or a combination thereof.

In some examples, the excipient comprises a dispersion enhancer. In some examples, the dispersion enhancer comprises starch, alginic acid, polyvinylpyrrolidones, guar gum, kaolin, bentonite, purified wood cellulose, sodium starch glycolate, isomorphous silicate, or microcrystalline cellulose, or any combination thereof as high HLB emulsifier surfactants.

In some examples, the excipient comprises a disintegrant. In some examples, a disintegrant comprises a non-effervescent disintegrant. In some examples, a non-effervescent disintegrants comprises starches such as corn starch, potato starch, pregelatinized and modified starches thereof, sweeteners, clays, such as bentonite, micro-crystalline cellulose, alginates, sodium starch glycolate, or gums such as agar, guar, locust bean, karaya, pectin, and tragacanth, or any combination thereof. In some examples, a disintegrant comprises an effervescent disintegrant. In some examples, a suitable effervescent disintegrant comprises bicarbonate in combination with citric acid, and sodium bicarbonate in combination with tartaric acid.

In some examples, the excipient comprises a sweetener, a flavoring agent or both. In some examples, a sweetener comprises glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like, or any combination thereof. In some cases, flavoring agents incorporated into a composition comprise synthetic flavor oils and flavoring aromatics; natural oils; extracts from plants, leaves, flowers, and fruits; or any combination thereof. In some embodiments, a flavoring agent comprises a cinnamon oil; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot, or any combination thereof.

In some examples, the excipient comprises a pH agent (e.g., to minimize oxidation or degradation of a component of the composition), a stabilizing agent (e.g., to prevent modification or degradation of a component of the composition), a buffering agent (e.g., to enhance temperature stability), a solubilizing agent (e.g., to increase protein solubility), or any combination thereof. In some examples, the excipient comprises a surfactant, a sugar, an amino acid, an antioxidant, a salt, a non-ionic surfactant, a solubilizer, a trigylceride, an alcohol, or any combination thereof. In some examples, the excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerine, xanthan rubber, soy isoflavones, polysorbate 80, ethyl alcohol, water, teprenone, or any combination thereof. In some examples, the excipient comprises a cryo-preservative. In some examples, the excipient comprises DMSO, glycerol, polyvinylpyrrolidone (PVP), or any combination thereof. In some examples, the excipient comprises a sucrose, a trehalose, a starch, a salt of any of these, a derivative of any of these, or any combination thereof.

In some examples, the pharmaceutical composition comprises a diluent. In some examples, the diluent comprises water, glycerol, methanol, ethanol, or other similar biocompatible diluents, or any combination thereof. In some examples, a diluent comprises an aqueous acid such as acetic acid, citric acid, maleic acid, hydrochloric acid, phosphoric acid, nitric acid, sulfuric acid, or any combination thereof. In some examples, a diluent comprises an alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, choline, lactose, maltose, mannitol, simethicone, sorbitol, starch, pregelatinized starch, talc, xylitol and/or anhydrates, hydrates and/or pharmaceutically acceptable derivatives thereof or combinations thereof.

In some examples, the pharmaceutical composition comprises a carrier. In some examples, the carrier comprises a liquid or solid filler, solvent, or encapsulating material. In some examples, the carrier comprises additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), alone or in combination.

### Administration

Administration can refer to methods that can be used to enable the delivery of a composition described herein (e.g., comprising an engineered guide RNA or an engineered polynucleotide encoding the same) to the desired site of biological action. For example, an engineered guide RNA or an expression cassette can be comprised in a DNA construct, a viral vector, or both and be administered by intravenous administration. Administration disclosed herein to an area in need of treatment or therapy can be achieved by, for example, and not by way of limitation, oral administration, topical administration, intravenous administration, inhalation administration, or any combination thereof. In some embodiments, delivery can include inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration, or a combination thereof. Delivery can include direct application to the affected tissue or region of the body. In some cases, topical administration can comprise administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin. Delivery can include a parenchymal injection, an intra-thecal injection, an intra-ventricular injection, or an intra-cisternal injection. A composition provided herein can be administered by any method. A method of administration can be by intra-arterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump, or a combination thereof. In some embodiments, delivery can be by an enema, an eye drop, a nasal spray, or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). In some embodiments, a medical professional can administer the composition.

In some cases, administering can be oral ingestion. In some cases, delivery can be a capsule or a tablet. Oral ingestion delivery can comprise a tea, an elixir, a food, a drink, a beverage, a syrup, a liquid, a gel, a capsule, a tablet, an oil, a tincture, or any combination thereof. In some embodiments, a food can be a medical food. In some instances, a capsule can comprise hydroxymethylcellulose. In some embodiments, a capsule can comprise a gelatin, hydroxypropylmethyl cellulose, pullulan, or any combination thereof. In some cases, capsules can comprise a coating, for example, an enteric coating. In some embodiments, a capsule can comprise a vegetarian product or a vegan product such as a hypromellose capsule. In some embodiments, delivery can comprise inhalation by an inhaler, a diffuser, a nebulizer, a vaporizer, or a combination thereof.

In some embodiments, disclosed herein can be a method, comprising administering a composition disclosed herein to a subject (e.g., a human) in need thereof. In some instances, the method can treat (including prevent) a disease in the subject.

In some examples, a pharmaceutical composition disclosed herein can be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

The appropriate dosage and treatment regimen for the methods of treatment described herein vary with respect to the particular disease being treated, the gRNA and/or ADAR (or a vector encoding the gRNA and/or ADAR) being delivered, and the specific condition of the subject. In some examples, the administration can be over a period of time until the desired effect (e.g., reduction in symptoms can be achieved). In some examples, administration can be 1, 2, 3, 4, 5, 6, or 7 times per week. In some examples, administration or application of a composition disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. In some examples, administration can be over a period of 1, 2, 3, 4, 5, 6, 7, 8, 9, or 10 weeks. In some examples, administration can be over a period of 2, 3, 4, 5, 6 or more months. In some examples, administration can be performed repeatedly over a lifetime of a subject, such as once a month or once a year for the lifetime of a subject. In some examples, administration can be performed repeatedly over a substantial portion of a subject's life, such as once a month or once a year for at least about 1 year, 5 years, 10 years, 15 years, 20 years, 25 years, 30 years, or more. In some examples, treatment can be resumed following a period of remission.

Pharmaceutical compositions for oral administration can be in tablet, capsule, powder, or liquid form. A tablet can include a solid carrier such as gelatin or an adjuvant. Liquid pharmaceutical compositions generally include a liquid carrier such as water, petroleum, animal or vegetable oils, mineral oil, or synthetic oil. Physiological saline solution, dextrose or other saccharide solution or glycols such as ethylene glycol, propylene glycol or polyethylene glycol can be included.

For intravenous, cutaneous, or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

In some embodiments, the polynucleotide of the present disclosure or recombinant polynucleotide cassette of the present disclosure may be administered to cells via a lipid nanoparticle. In some embodiments, the lipid nanoparticle may be administered at the appropriate concentration according to standard methods appropriate for the target cells.

In some embodiments, the polynucleotide of the present disclosure or recombinant polynucleotide cassette of the present disclosure may be administered to cells via a viral vector. In some embodiments, the viral vector may be administered at the appropriate multiplicity of infection according to standard transduction methods appropriate for the target cells. Titers of the virus vector or capsid to administer can vary depending on the target cell type or cell state and number and can be determined by those of skill in the art. In some embodiments, at least about 10² infections units are administered. In some embodiments, at least about 10³, 10⁴, 10⁵, 10⁶, 10⁷, 10⁸, 10⁹, 10¹⁰, 10¹¹, 10¹², or 10¹³ infectious units are administered.

In some embodiments, the polynucleotide or recombinant polynucleotide cassette is introduced to cells of any type or state, including, but not limited to neural cells, cells of the eye (including retinal cells, retinal pigment epithelium, and corneal cells), lung cells, epithelial cells, skeletal muscle cells, dendritic cells, hepatic cells, pancreatic cells, bone cells, hematopoietic stem cells, spleen cells, keratinocytes, fibroblasts, endothelial cells, prostate cells, and heart cells.

In some embodiments, the polynucleotide or the disclosure or the recombinant polynucleotide cassette of the disclosure may be introduced to cells in vitro via a viral vector for administration of modified cells to a subject. In some embodiments, a viral vector encoding the polynucleotide of the disclosure or the recombinant polynucleotide cassette of the disclosure is introduced to cells that have been removed from a subject. In some embodiments, the modified cells are placed back in the subject following introduction of the viral vector.

In some embodiments, a dose of modified cells is administered to a subject according to the age and species of the subject, disease or disorder to be treated, as well as the cell type or state and mode of administration. In some embodiments, at least about 10²-10⁸ cells are administered per dose. In some embodiments, cells transduced with viral vector are administered to a subject in an effective amount.

In some embodiments, the dose of viral vector administered to a subject will vary according to the age of the subject, the disease or disorder to be treated, and mode of administration. In some embodiments, the dose for achieving a therapeutic effect is a virus titer of at least about 10², 103, 104, 10¹, 10⁶, 107, 10¹, 10¹, 10¹⁰, 10¹¹, 10¹², 10¹³, 1014, 10¹¹, 10¹⁶ or more transducing units.

Administration of the pharmaceutically useful polynucleotide of the present disclosure or the polynucleotide cassette of the present disclosure is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g., decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

Unless defined otherwise, all terms of art, notations and other technical and scientific terms or terminology used herein is intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%, 97%, 98%, 99%, or 100% over a region of 10, 15, 20, 25, 30, 35, 40, 45, 50, or more nucleotides, or can refer to two nucleic acids that hybridize under stringent conditions (i.e., stringent hybridization conditions). Nucleic acids can include nonspecific sequences. As used herein, the term “nonspecific sequence” or “not specific” can refer to a nucleic acid sequence that contains a series of residues that can be not designed to be complementary to or can be only partially complementary to any other nucleic acid sequence.

The terms “determining,” “measuring,” “evaluating,” “assessing,” “assaying,” and “analyzing” can be used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The term “encode,” as used herein, refers to an ability of a polynucleotide to provide information or instructions sequence sufficient to produce a corresponding gene expression product. In a non-limiting example, mRNA can encode a polypeptide during translation, whereas DNA can encode a mRNA molecule during transcription.

As used herein, the term “facilitates RNA editing” by an engineered guide RNA refers to the ability of the engineered guide RNA when associated with an RNA editing entity and a target RNA to provide a targeted edit of the target RNA by the RNA edited entity. In some instances, the engineered guide RNA can directly recruit or position/orient the RNA editing entity to the proper location for editing of the target RNA. In other instances, the engineered guide RNA when hybridized to the target RNA forms a guide-target RNA scaffold with one or more structural features as described herein, where the guide-target RNA scaffold with structural features recruits or positions/orients the RNA editing entity to the proper location for editing of the target RNA.

As used herein, the term “engineered guide RNA” can be used interchangeable with “guide RNA” and refers to a designed polynucleotide that is at least partially complementary to a target RNA. An engineered guide RNA of the present disclosure can be used to facilitate modification of the target RNA. Modification of the target RNA includes alteration of RNA splicing, reduction or enhancement of protein translation, target RNA knockdown, target RNA degradation, and/or ADAR mediated RNA editing of the target RNA. In some cases, guide RNAs facilitate ADAR mediated RNA editing for the purpose of target mRNA knockdown, downstream protein translation reduction or inhibition, downstream protein translation enhancement, correction of mutations (including correction of any G to A mutation, such as missense or nonsense mutations), introduction of mutations (e.g., introduction of an A to I (read as a G by cellular machinery) substitution), or alter the function of any adenosine containing a regulatory motif (e.g., polyadenylation signal, miRNA binding site, etc.). In some cases, a guide RNA can effect a functional outcome (e.g., target RNA modulation, downstream protein translation) via a combination of mechanisms, for example, ADAR-mediated RNA editing and binding and/or degrading target RNA. In some cases, a guide RNA can facilitate introduction of mutations at sites targeted by enzymes in order to modify the affinity of such enzymes for targeting and cleaving such sites. The guide RNAs of this disclosure can contain one or more structural features. A structural feature can be formed from latent structure in latent (unbound) guide RNA upon hybridization of the engineered latent guide RNA to a target RNA. Latent structure refers to a structural feature that forms or substantially forms only upon hybridization of a guide RNA to a target RNA. For example, upon hybridization of the guide RNA to the target RNA, the latent structural feature is formed in the resulting double stranded RNA (also referred herein as guide-target RNA scaffold). In such cases, a structural feature can include, but is not limited to, a mismatch, a wobble base pair, a symmetric internal loop, an asymmetric internal loop, a symmetric bulge, or an asymmetric bulge. In other instances, a structural feature can be a pre-formed structure (e.g., a GluR2 recruitment hairpin, or a hairpin from U7 snRNA).

A “guide-target RNA scaffold,” as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more structural features selected from a bulge, mismatch, internal loop, hairpin, or wobble base pair.

“Messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As disclosed herein, a “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a “bulge” or an “internal loop,” depending on the size of the structural feature.

The term “structured motif” refers to a combination of two or more structural features in a guide-target RNA scaffold.

The terms “subject,” “individual,” or “patient” can be used interchangeably herein. A “subject” refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some cases, the subject may not be necessarily diagnosed or suspected of being at high risk for the disease

The term “in vivo” refers to an event that takes place in a subject's body.

The term “ex vivo” refers to an event that takes place outside of a subject's body. An ex vivo assay may not be performed on a subject. Rather, it can be performed upon a sample separate from a subject. An example of an ex vivo assay performed on a sample can be an “in vitro” assay.

The term “in vitro” refers to an event that takes places contained in a container for holding laboratory reagent such that it can be separated from the biological source from which the material can be obtained. In vitro assays can encompass cell-based assays in which living or dead cells can be employed. In vitro assays can also encompass a cell-free assay in which no intact cells can be employed.

The term “wobble base pair” refers to two bases that weakly pair. For example, a wobble base pair can refer to a G paired with a U.

The term “substantially forms” as described herein, when referring to a particular secondary structure, refers to formation of at least 80% of the structure under physiological conditions (e.g., physiological pH, physiological temperature, physiological salt concentration, etc.).

As used herein, the term “therapeutic polynucleotide” may to a polynucleotide that is introduced into a cell and is capable of being expressed in the cell or to a polynucleotide that may, in itself, have a therapeutic activity, such as a gRNA or a tRNA.

As used herein, the term “polynucleotide” refers to a single or double-stranded polymer of deoxyribonucleotide (DNA) or ribonucleotide (RNA) bases read from the 5′ to the 3′ end. The term “RNA” is inclusive of dsRNA (double stranded RNA), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), rRNA (ribosomal RNA), snoRNA (small nucleolar RNA), and cRNA (complementary RNA). The term DNA is inclusive of cDNA, genomic DNA, and DNA-RNA hybrids. A sequence of a polynucleotide may be provided interchangeably as an RNA sequence (containing U) or a DNA sequence (containing T). A sequence provided as an RNA sequence is intended to also cover the corresponding DNA sequence and the reverse complement RNA sequence or DNA sequence. A sequence provided as a DNA sequence is intended to also cover the corresponding RNA sequence and the reverse complement RNA sequence or DNA sequence.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together—optionally to preserve the conformation of the fused protein domains, prevent unfavorable interactions between the fused protein domains which can compromise their respective functions, or both.

The term “ameliorating” refers to any therapeutically beneficial result in the treatment of a disease state, e.g., Rett syndrome, including prophylaxis, lessening in the severity or progression, remission, or cure thereof.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence (also called the subject sequence) to which test sequences (also called query sequences) are compared. The percent sequence identity is defined as a test sequence's percent identity to a reference sequence. For example, when stated “Sequence A having a sequence identity of 50% to Sequence B,” Sequence A is the test sequence and Sequence B is the reference sequence. When using a sequence comparison algorithm, test and reference sequences are input into a computer program, subsequence coordinates are designated, if necessary, and sequence algorithm program parameters are designated. The sequence comparison algorithm then aligns the sequences to achieve the maximum alignment, based on the designated program parameters, introducing gaps in the alignment if necessary. The percent sequence identity for the test sequence(s) relative to the reference sequence can then be determined from the alignment of the test sequence to the reference sequence. The equation for percent sequence identity from the aligned sequence is as follows:

[(Number of Identical Positions)/(Total Number of Positions in the Test Sequence)]×100%

For purposes herein, percent identity and sequence similarity calculations are performed using the BLAST algorithm for sequence alignment, which is described in Altschul et al., J. Mol. Biol. 215:403-410 (1990). Software for performing BLAST analyses is publicly available through the National Center for Biotechnology Information (www.ncbi.nlm.nih.gov/). The BLAST algorithm uses a test sequence (also called a query sequence) and a reference sequence (also called a subject sequence) to search against, or in some cases, a database of multiple reference sequences to search against. The BLAST algorithm performs sequence alignment by finding high-scoring alignment regions between the test and the reference sequences by scoring alignment of short regions of the test sequence (termed “words”) to the reference sequence. The scoring of each alignment is determined by the BLAST algorithm and takes factors into account, such as the number of aligned positions, as well as whether introduction of gaps between the test and the reference sequences would improve the alignment. The alignment scores for nucleic acids can be scored by set match/mismatch scores. For protein sequences, the alignment scores can be scored using a substitution matrix to evaluate the significance of the sequence alignment, for example, the similarity between aligned amino acids based on their evolutionary probability of substitution. For purposes herein, the substitution matrix used is the BLOSUM62 matrix. For purposes herein, the public default values of Apr. 6, 2023, are used when using the BLASTN and BLASTP algorithms. The BLASTN and BLASTP algorithms then output a “Percent Identity” output value and a “Query Coverage” output value. The overall percent sequence identity as used herein can then be calculated from the BLASTN or BLASTP output values as follows:

Percent Sequence Identity=(“Percent Identity” output value)×(“Query Coverage” output value)

The following non-limiting examples illustrate the calculation of percent identity between two nucleic acids sequences. The percent identity is calculated as follows: [(number of identical nucleotide positions)/(total number of nucleotides in the test sequence)]×100%. Percent identity is calculated to compare test sequence 1: AAAAAGGGGG (SEQ ID NO: 1276) (length=10 nucleotides) to reference sequence 2: AAAAAAAAAA (SEQ ID NO: 1277) (length=10 nucleotides). The percent identity between test sequence 1 and reference sequence 2 would be [(5)/(10)]×100%=50%. Test sequence 1 has 50% sequence identity to reference sequence 2. In another example, percent identity is calculated to compare test sequence 3: CCCCCGGGGGGGGGGCCCCC (SEQ ID NO: 1278) (length=20 nucleotides) to reference sequence 4: GGGGGGGGGG (SEQ ID NO: 1279) (length=10 nucleotides). The percent identity between test sequence 3 and reference sequence 4 would be [(10)/(20)]×100%=50%. Test sequence 3 has 50% sequence identity to reference sequence 4. In another example, percent identity is calculated to compare test sequence 5: GGGGGGGGGG (SEQ ID NO:1279) (length=10 nucleotides) to reference sequence 6: CCCCCGGGGGGGGGGCCCCC (SEQ ID NO: 1278) (length=20 nucleotides). The percent identity between test sequence 5 and reference sequence 6 would be [(10)/(10)]×100%=100%. Test sequence 5 has 100% sequence identity to reference sequence 6.

The following non-limiting examples illustrate the calculation of percent identity between two protein sequences. The percent identity is calculated as follows: [(number of identical amino acid positions)/(total number of amino acids in the test sequence)]×100%. Percent identity is calculated to compare test sequence 7: FFFFFYYYYY (SEQ ID NO:1280) (length=10 amino acids) to reference sequence 8: YYYYYYYYYY (SEQ ID NO: 1281) (length=10 amino acids). The percent identity between test sequence 7 and reference sequence 8 would be [(5)/(10)]×100%=50%. Test sequence 7 has 50% sequence identity to reference sequence 8. In another example, percent identity is calculated to compare test sequence 9: LLLLLFFFFFYYYYYLLLLL (SEQ ID NO: 1282) (length=20 amino acids) to reference sequence 10: FFFFFYYYYY (SEQ ID NO: 1280) (length=10 amino acids). The percent identity between test sequence 9 and reference sequence 10 would be [(10)/(20)]×100%=50%. Test sequence 9 has 50% sequence identity to reference sequence 10. In another example, percent identity is calculated to compare test sequence 11: FFFFFYYYYY (SEQ ID NO: 1280) (length=10 amino acids) to reference sequence 12: LLLLLFFFFFYYYYYLLLLL (SEQ ID NO:1282) (length=20 amino acids). The percent identity between test sequence 11 and reference sequence 12 would be [(10)/(10)]×100%=100%. Test sequence 11 has 100% sequence identity to reference sequence 12.

As used herein, the term “subject” broadly refers to any animal, including but not limited to, human and non-human animals (e.g., dogs, cats, cows, horses, sheep, pigs, poultry, fish, crustaceans, etc.).

As used herein, the term “effective amount” refers to the amount of a composition (e.g., a synthetic peptide) sufficient to effect beneficial or desired results. An effective amount can be administered in one or more administrations, applications or dosages and is not intended to be limited to a particular formulation or administration route.

As used herein, the term “therapeutically effective amount” is an amount that is effective to ameliorate a symptom of a disease. A therapeutically effective amount can be a “prophylactically effective amount” as prophylaxis can be considered therapy.

As used herein, the terms “administration” and “administering” refer to the act of giving a drug, prodrug, or other agent, or therapeutic treatment (e.g., peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal or lingual), ear, rectal, vaginal, by injection (e.g., intravenously, subcutaneously, intratumorally, intraperitoneally, etc.) and the like.

As used herein, the term “treatment” or “treating” means an approach to obtaining a beneficial or intended clinical result. The beneficial or intended clinical result can include a therapeutic benefit and/or a prophylactic benefit, alleviation of symptoms, a reduction in the severity of the disease, inhibiting an underlying cause of a disease or condition, steadying diseases in a non-advanced state, delaying the progress of a disease, and/or improvement or alleviation of disease conditions. Also, a therapeutic benefit can be achieved with the eradication or amelioration of one or more of the physiological symptoms associated with the underlying disorder such that an improvement can be observed in the subject, notwithstanding that the subject can still be afflicted with the underlying disorder. A prophylactic effect includes delaying, preventing, or eliminating the appearance of a disease or condition, delaying or eliminating the onset of one or more symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

As used herein, the term “pharmaceutical composition” refers to the combination of an active ingredient with a carrier, inert or active, making the composition especially suitable for therapeutic or diagnostic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), glycerol, liquid polyethylene glycols, aprotic solvents such as dimethylsulfoxide, N-methylpyrrolidone and mixtures thereof, and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 2^{st} Ed., Mack Publ. Co., Easton, Pa. (2005), incorporated herein by reference in its entirety.

Throughout this application, various embodiments are presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the singular forms “a,” “an” and “the” include plural referents unless the context clearly dictates otherwise.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

### NUMBERED EMBODIMENTS

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. An expression cassette comprising: a promoter sequence comprising: a zinc finger 143 motif, an OCT-1 transcription factor binding sequence, a proximal sequence element; a payload sequence under transcriptional control of the promoter sequence, the payload sequence comprising a small RNA payload; and a transcription termination sequence; wherein the expression cassette comprises one or more sequence elements selected from the group consisting of: a) the zinc finger 143 motif having at least 80% sequence identity to any one of SEQ ID NO: 24-SEQ ID NO: 26, b) the OCT-1 transcription factor binding sequence having at least 80% sequence identity to any one of SEQ ID NO: 27-SEQ ID NO: 30, c) the proximal sequence element having at least 80% sequence identity to any one of SEQ ID NO: 31-SEQ ID NO: 37, and d) combinations thereof. 2. The expression cassette of embodiment 1, wherein the zinc finger 143 motif comprises at least 80%, at least 85%, at least 90%, at least 95%, or 100% sequence identity to any one of SEQ ID NO: 24-SEQ ID NO: 26. 3. The expression cassette of embodiment 1, wherein the zinc finger 143 motif comprises at least 80%, at least 85%, at least 90%, at least 95%, or 100% sequence identity to SEQ ID NO: 20. 4. The expression cassette of any one of embodiments 1-3, wherein the OCT-1 transcription factor binding sequence comprises at least 80%, at least 85%, at least 90%, at least 95%, or 100% sequence identity to any one of SEQ ID NO: 27-SEQ ID NO: 30. 5. The expression cassette of any one of embodiments 1-4, wherein the proximal sequence element comprises at least 80%, at least 85%, at least 90%, at least 95%, or 100% sequence identity to any one of SEQ ID NO: 31-SEQ ID NO: 37. 6. The expression cassette of any one of embodiments 1-5, wherein the transcription termination sequence comprises at least 80%, at least 85%, at least 90%, at least 95%, or 100% sequence identity to any one of SEQ ID NO: 40-SEQ ID NO: 42. 7. The expression cassette of any one of embodiments 1-6, wherein the transcription termination sequence comprises at least 80%, at least 85%, at least 90%, at least 95%, or 100% sequence identity to SEQ ID NO: 60 SEQ ID NO: 1242-SEQ ID NO: 1247, or SEQ ID NO: 1254-SEQ ID NO: 1257. 8. The expression cassette of any one of embodiments 1-7, wherein the transcription termination sequence comprises at least 80%, at least 85%, at least 90%, at least 95%, or 100% sequence identity to SEQ ID NO: 1242. 9. The expression cassette of any one of embodiments 1-7, wherein the transcription termination sequence comprises a sequence of SEQ ID NO: 1242. 10. The expression cassette of any one of embodiments 1-7, wherein the transcription termination sequence comprises at least 80%, at least 85%, at least 90%, at least 95%, or 100% sequence identity to SEQ ID NO: 60. 11. The expression cassette of any one of embodiments 1-7, wherein the transcription termination sequence comprises a sequence of SEQ ID NO: 60. 12. The expression cassette of any one of embodiments 1-11, wherein the transcription termination sequence comprises a sequence of SEQ ID NO: 38 or SEQ ID NO: 39. 13. An expression cassette comprising: a promoter sequence comprising a proximal sequence element, wherein the promoter sequence comprises a sequence having at least 75% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253 in which the proximal sequence element of the promoter sequence is replaced with a sequence of any one of SEQ ID NO: 67-SEQ ID NO: 120; a payload sequence under transcriptional control of the promoter sequence, the payload sequence comprising a small RNA payload; and a transcription termination sequence comprising a 3′ box sequence element, wherein the transcription termination sequence comprises a sequence having at least 75% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257 in which the 3′ box sequence element of the termination sequence is replaced with a sequence of any one of SEQ ID NO: 121-SEQ ID NO: 166. 14. The expression cassette of embodiment 13, wherein the promoter sequence comprises a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253 in which the proximal sequence element of the promoter sequence is replaced with a sequence of any one of SEQ ID NO: 67-SEQ ID NO: 120. 15. The expression cassette of embodiment 13 or embodiment 14, wherein the promoter sequence comprises a sequence having at least 90% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253 in which the proximal sequence element of the promoter sequence is replaced with a sequence of any one of SEQ ID NO: 67-SEQ ID NO: 120. 16. The expression cassette of any one of embodiments 13-15, wherein the termination sequence comprises a sequence having at least 80% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257 in which the 3′ box sequence element of the termination sequence is replaced with a sequence of any one of SEQ ID NO: 121-SEQ ID NO: 166. 17. The expression cassette of any one of embodiments 13-16, wherein the termination sequence comprises a sequence having at least 90% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257 in which the 3′ box sequence element of the termination sequence is replaced with a sequence of any one of SEQ ID NO: 121-SEQ ID NO: 166. 18. An expression cassette comprising: a promoter sequence comprising a sequence having at least 75% sequence identity to any one of SEQ ID NO: 16-SEQ ID NO: 17, SEQ ID NO: 167-SEQ ID NO: 707, SEQ ID NO: 1241, SEQ ID NO: 1248-SEQ ID NO: 1253; a payload sequence under transcriptional control of the promoter sequence, the payload sequence comprising a small RNA payload; and a transcription termination sequence comprising a sequence having at least 75% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257. 19. The expression cassette of embodiment 18, wherein the promoter sequence comprises a sequence having at least 80% sequence identity to any one of SEQ ID NO: 16-SEQ ID NO: 17, SEQ ID NO: 167-SEQ ID NO: 707, SEQ ID NO: 1241, SEQ ID NO: 1248-SEQ ID NO: 1253. 20. The expression cassette of embodiment 18 or embodiment 19, wherein the promoter sequence comprises a sequence having at least 90% sequence identity to any one of SEQ ID NO: 16-SEQ ID NO: 17, SEQ ID NO: 167-SEQ ID NO: 707, SEQ ID NO: 1241, SEQ ID NO: 1248-SEQ ID NO: 1253. 21. The expression cassette of any one of embodiments 18-20, wherein the termination sequence comprises a sequence having at least 80% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257. 22. The expression cassette of any one of embodiments 18-21, wherein the termination sequence comprises a sequence having at least 90% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257. 23. The expression cassette of any one of embodiments 13-22, wherein the promoter sequence is SEQ ID NO: 376. 24. The expression cassette of any one of embodiments 13-22, wherein the promoter sequence is SEQ ID NO: 1250. 25. The expression cassette of embodiment 23 or embodiment 24, wherein the transcription termination sequence is SEQ ID NO: 917. 26. The expression cassette of embodiment 23 or embodiment 24, wherein the transcription termination sequence is SEQ ID NO: 1254. 27. The expression cassette of any one of embodiments 13-22, wherein the promoter sequence is SEQ ID NO: 168. 28. The expression cassette of any one of embodiments 13-22, wherein the promoter sequence is SEQ ID NO: 1251. 29. The expression cassette of embodiment 27 or embodiment 28, wherein the transcription termination sequence is SEQ ID NO: 709. 30. The expression cassette of embodiment 27 or embodiment 28, wherein the transcription termination sequence is SEQ ID NO: 1255. 31. The expression cassette of any one of embodiments 13-22, wherein the promoter sequence is SEQ ID NO: 1241. 32. The expression cassette of embodiment 31, wherein the transcription termination sequence is SEQ ID NO: 1242 or SEQ ID NO: 60. 33. The expression cassette of any one of embodiments 13-22, wherein the promoter sequence is SEQ ID NO: 17. 34. The expression cassette of embodiment 33, wherein the transcription termination sequence is SEQ ID NO: 1242 or SEQ ID NO: 60. 35. The expression cassette of any one of embodiments 1-34, wherein the small RNA payload comprises an engineered guide RNA capable of hybridizing to a target sequence. 36. The expression cassette of any one of embodiments 1-35, wherein the engineered guide RNA is at least 70%, at least 75%, at least 80%, at least 85%, at least 90%, at least 95%, or 100% reverse complementary to the target sequence. 37. The expression cassette of embodiment 35 or embodiment 36, wherein the engineered guide RNA comprises at least one base pair mismatch relative to the target sequence. 38. The expression cassette of any one of embodiments 35-37, wherein the target sequence comprises an adenosine residue. 39. The expression cassette of any one of embodiments 35-38, wherein the target sequence is an RNA sequence. 40. The expression cassette of embodiment 39, wherein the RNA sequence is a mRNA or a pre-mRNA. 41. The expression cassette of any one of embodiments 35-40, wherein the target sequence comprises a G to A mutation relative to a wild type sequence. 42. The expression cassette of any one of embodiments 35-41, wherein the target sequence comprises a missense mutation or a nonsense mutation relative to a wild type sequence. 43. The expression cassette of any one of embodiments 35-42, wherein the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2). 44. The expression cassette of any one of embodiments 35-43, wherein the payload sequence comprises at least 80%, at least 85%, at least 90%, at least 95%, or 100% sequence identity to SEQ ID NO: 1273, SEQ ID NO: 1274, or SEQ ID NO: 61. 45. The expression cassette of any one of embodiments 1-44, wherein the small RNA payload comprises an antisense oligonucleotide, an siRNA, an shRNA, a miRNA, or a tracrRNA. 46. The expression cassette of any one of embodiments 1-45, wherein the small RNA payload is not less than 20 nucleotide residues and not more than 500 nucleotide residues long. 47. The expression cassette of any one of embodiments 1-46, wherein the small RNA payload is not less than 60 and not more than 100 residues long. 48. The expression cassette of any one of embodiments 1-47, wherein the small RNA payload is not less than 80 and not more than 120 residues long. 49. The expression cassette of any one of embodiments 1-48, wherein the small RNA payload is not less than 100 and not more than 140 residues long. 50. The expression cassette of any one of embodiments 1-49, wherein the small RNA payload is not less than 130 and not more than 170 residues long. 51. The expression cassette of any one of embodiments 1-50, wherein the payload sequence further comprises an Sm binding sequence or a hairpin sequence. 52. The expression cassette of embodiment 51, wherein the hairpin sequence comprises a U7 hairpin. 53. The expression cassette of embodiment 51 or embodiment 52, wherein the hairpin sequence comprises at least 80%, at least 85%, at least 90%, at least 95%, or 100% sequence identity to SEQ ID NO: 52, SEQ ID NO: 54, SEQ ID NO: 56, or SEQ ID NO: 58. 54. The expression cassette of any one of embodiments 1-53, wherein the expression cassette comprises two or more of the sequence elements. 55. The expression cassette of any one of embodiments 1-54, wherein the expression cassette comprises three or more of the sequence elements. 56. The expression cassette of any one of embodiments 1-55, wherein the expression cassette has a length of not less than 1300 nucleotide residues and not more than 2160 nucleotide residues. 57. The expression cassette of any one of embodiments 1-56, wherein the expression cassette comprises at least 80% sequence identity to a U1 sequence or a U7 sequence. 58. The expression cassette of embodiment 57, wherein the U1 sequence is a mouse U1 sequence or a human U1 sequence. 59. The expression cassette of embodiment 57, wherein the U7 sequence is a mouse U7 sequence or a human U7 sequence. 60. The expression cassette of any one of embodiments 1-59, wherein the promoter sequence comprises at least 80%, at least 85%, at least 90%, at least 95%, or 100% sequence identity to any one of SEQ ID NO: 13-SEQ ID NO: 17, SEQ ID NO: 1241, or SEQ ID NO: 1248-SEQ ID NO: 1253. 61. The expression cassette of any one of embodiments 1-60, wherein the promoter sequence comprises at least 80%, at least 85%, at least 90%, at least 95%, or 100% sequence identity to SEQ ID NO: 1241. 62. The expression cassette of any one of embodiments 1-60, wherein the promoter sequence comprises a sequence of SEQ ID NO: 1241. 63. The expression cassette of embodiment 62, wherein the transcription termination sequence is SEQ ID NO: 1242 or SEQ ID NO: 60. 64. The expression cassette of any one of embodiments 1-60, wherein the promoter sequence comprises at least 80%, at least 85%, at least 90%, at least 95%, or 100% sequence identity to SEQ ID NO: 17. 65. The expression cassette of any one of embodiments 1-60, wherein the promoter sequence comprises a sequence of SEQ ID NO: 17. 66. The expression cassette of embodiment 64 or embodiment 65, wherein the transcription termination sequence is SEQ ID NO: 1242 or SEQ ID NO: 60. 67. The expression cassette of any one of embodiments 1-66, wherein the expression cassette comprises at least 80%, at least 85%, at least 90%, at least 95%, or 100% sequence identity to any one of SEQ ID NO: 1-SEQ ID NO: 12 or SEQ ID NO: 59. 68. The expression cassette of any one of embodiments 1-67, wherein the zinc finger 143 motif is capable of recruiting a ZNF143 transcription factor. 69. The expression cassette of any one of embodiments 1-68, wherein the OCT-1 transcription factor binding sequence is capable of recruiting an OCT-1 transcription factor. 70. The expression cassette of any one of embodiments 1-69, wherein the proximal sequence element is capable of recruiting a SNAPc. 71. The expression cassette of any one of embodiments 1-70, wherein the proximal sequence element is capable of integrator dependent recruitment of RNA polymerase II. 72. The expression cassette of any one of embodiments 1-71, wherein the small RNA payload is capable of forming a guide-target RNA scaffold comprising a structural feature upon hybridization of the small RNA payload to a target sequence. 73. The expression cassette of embodiment 72, wherein the structural feature is a bulge, a mismatch, an internal loop, a hairpin, or combinations thereof. 74. The expression cassette of embodiment 73, wherein the structural feature comprises the bulge, and wherein the bulge is a symmetric bulge. 75. The expression cassette of embodiment 73, wherein the structural feature comprises the bulge, and wherein the bulge is an asymmetric bulge. 76. The expression cassette of embodiment 73, wherein the structural feature comprises the internal loop, and wherein the internal loop is a symmetric internal loop. 77. The expression cassette of embodiment 73, wherein the structural feature comprises the internal loop, and wherein the internal loop is an asymmetric internal loop. 78. The expression cassette of embodiment 73, wherein the structural feature comprises the hairpin, and wherein the hairpin is a recruitment hairpin or a non-recruitment hairpin. 79. The expression cassette of any one of embodiments 72-78, wherein the guide-target RNA scaffold comprises a Wobble base pair. 80. A method of expressing a small RNA payload in a cell, the method comprising delivering the expression cassette of any one of embodiments 1-79 to a cell and expressing the small RNA payload encoded by the expression cassette in the cell. 81. A method of editing a target sequence, the method comprising: delivering an expression cassette to a cell encoding the target sequence, wherein the expression cassette comprises: a promoter sequence comprising: a zinc finger 143 motif, an OCT-1 transcription factor binding sequence, and a proximal sequence element, a payload sequence under transcriptional control of the promoter sequence, the payload sequence comprising a small RNA payload, wherein the small RNA payload comprises an engineered guide RNA sequence capable of hybridizing to the target sequence, and a transcription termination sequence; expressing the small RNA payload in the cell; forming a guide-target RNA scaffold upon hybridization of the small RNA payload to the target sequence; recruiting an editing enzyme to the target sequence; and editing the target sequence with the editing enzyme. 82. A method of editing a target sequence, the method comprising: delivering an expression cassette to a cell encoding the target sequence, wherein the expression cassette comprises: a promoter sequence comprising a proximal sequence element, wherein the promoter sequence comprises a sequence having at least 75% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253 in which the proximal sequence element of the promoter sequence is replaced with a sequence of any one of SEQ ID NO: 67-SEQ ID NO: 120; a payload sequence under transcriptional control of the promoter sequence, the payload sequence comprising a small RNA payload; and a transcription termination sequence comprising a 3′ box sequence element, wherein the transcription termination sequence comprises a sequence having at least 75% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257 in which the 3′ box sequence element of the termination sequence is replaced with a sequence of any one of SEQ ID NO: 121-SEQ ID NO: 166; expressing the small RNA payload in the cell; forming a guide-target RNA scaffold upon hybridization of the small RNA payload to the target sequence; recruiting an editing enzyme to the target sequence; and editing the target sequence with the editing enzyme. 83. A method of editing a target sequence, the method comprising: delivering an expression cassette to a cell encoding the target sequence, wherein the expression cassette comprises: a promoter sequence comprising a proximal sequence element, wherein the promoter sequence comprises a sequence having at least 75% sequence identity to any one of SEQ ID NO: 16-SEQ ID NO: 17, SEQ ID NO: 167-SEQ ID NO: 707, SEQ ID NO: 1241, SEQ ID NO: 1248-SEQ ID NO: 1253; a payload sequence under transcriptional control of the promoter sequence, the payload sequence comprising a small RNA payload; and a transcription termination sequence comprising a 3′ box sequence element, wherein the transcription termination sequence comprises a sequence having at least 75% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257; expressing the small RNA payload in the cell; forming a guide-target RNA scaffold upon hybridization of the small RNA payload to the target sequence; recruiting an editing enzyme to the target sequence; and editing the target sequence with the editing enzyme. 84. The method of embodiment 82 or embodiment 83, wherein the promoter sequence is SEQ ID NO: 376. 85. The method of embodiment 82 or embodiment 83, wherein the promoter sequence is SEQ ID NO: 1250. 86. The method of embodiment 84 or embodiment 85, wherein the transcription termination sequence is SEQ ID NO: 917. 87. The method of embodiment 84 or embodiment 85, wherein the transcription termination sequence is SEQ ID NO: 1254. 88. The method of embodiment 82 or embodiment 83, wherein the promoter sequence is SEQ ID NO: 168. 89. The method of embodiment 82 or embodiment 83, wherein the promoter sequence is SEQ ID NO: 1251. 90. The method of embodiment 88 or embodiment 89, wherein the transcription termination sequence is SEQ ID NO: 709. 91. The method of embodiment 88 or embodiment 89, wherein the transcription termination sequence is SEQ ID NO: 1255. 92. The method of embodiment 82 or embodiment 83, wherein the promoter sequence is SEQ ID NO: 1241. 93. The method of embodiment 92, wherein the transcription termination sequence is SEQ ID NO: 1242 or SEQ ID NO: 60. 94. The method of embodiment 82 or embodiment 83, wherein the promoter sequence is SEQ ID NO: 17. 95. The method of embodiment 94, wherein the transcription termination sequence is SEQ ID NO: 1242 or SEQ ID NO: 60. 96. A method of editing a target sequence, the method comprising: delivering the expression cassette of any one of embodiments 1-79 to a cell encoding the target sequence; expressing the small RNA payload in the cell, wherein the small RNA payload comprises an engineered guide RNA capable of hybridizing to a target sequence; forming a guide-target RNA scaffold upon hybridization of the small RNA payload to the target sequence; recruiting an editing enzyme to the target sequence; and editing the target sequence with the editing enzyme. 97. The method of any one of embodiments 81-96, wherein the target sequence comprises a mutation relative to a wild type sequence. 98. The method of embodiment 97, wherein editing the target sequence corrects the mutation in the target sequence. 99. The method of embodiment 97 or embodiment 98, wherein the mutation is a missense mutation. 100. The method of embodiment 97 or embodiment 98, wherein the mutation is a nonsense mutation. 101. The method of any one of embodiments 97-100, wherein the mutation is a G to A mutation. 102. The method of any one of embodiments 97-101, wherein the mutation is associated with a disease. 103. The method of embodiment 102, wherein the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease. 104. The method of any one of embodiments 81-103, wherein the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2). 105. The method of embodiment 81-104, wherein editing the target sequence comprises editing an untranslated region of the target. 106. The method of embodiment 105, wherein the untranslated region is a 5′ untranslated region or a 3′ untranslated region. 107. The method of embodiment 106, wherein the 3′ untranslated region is a polyadenylation sequence. 108. The method of any one of embodiments 81-107, wherein editing the target sequence comprises editing a translation initiation site. 109. The method of any one of embodiments 81-107, wherein editing the target sequence alters expression of the target sequence. 110. The method of embodiment 109, wherein editing the target sequence increases expression of the target sequence. 111. The method of embodiment 109, wherein editing the target sequence decreases expression of the target sequence. 112. A method of treating a disease in a subject, the method comprising: administering to the subject a composition comprising an expression cassette comprising: a promoter sequence comprising: a zinc finger 143 motif, an OCT-1 transcription factor binding sequence, and a proximal sequence element, and a payload sequence under transcriptional control of the promoter sequence, the payload sequence comprising a small RNA payload; delivering the expression cassette to a cell of the subject; and expressing the small RNA payload in the cell, thereby treating the disease. 113. A method of treating a disease in a subject, the method comprising: administering to the subject a composition comprising an expression cassette comprising: a promoter sequence comprising a proximal sequence element, wherein the promoter sequence comprises a sequence having at least 75% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253 in which the proximal sequence element of the promoter sequence is replaced with a sequence of any one of SEQ ID NO: 67-SEQ ID NO: 120; a payload sequence under transcriptional control of the promoter sequence, the payload sequence comprising a small RNA payload; and a transcription termination sequence comprising a 3′ box sequence element, wherein the transcription termination sequence comprises a sequence having at least 75% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257 in which the 3′ box sequence element of the termination sequence is replaced with a sequence of any one of SEQ ID NO: 121-SEQ ID NO: 166; delivering the expression cassette to a cell of the subject; and expressing the small RNA payload in the cell, thereby treating the disease. 114. A method of treating a disease in a subject, the method comprising: administering to the subject a composition comprising an expression cassette comprising: a promoter sequence comprising a proximal sequence element, wherein the promoter sequence comprises a sequence having at least 75% sequence identity to any one of SEQ ID NO: 16-SEQ ID NO: 17, SEQ ID NO: 167-SEQ ID NO: 707, SEQ ID NO: 1241, SEQ ID NO: 1248-SEQ ID NO: 1253; a payload sequence under transcriptional control of the promoter sequence, the payload sequence comprising a small RNA payload; and a transcription termination sequence comprising a 3′ box sequence element, wherein the transcription termination sequence comprises a sequence having at least 75% sequence identity to any one of SEQ ID NO: 60, SEQ ID NO: 708-SEQ ID NO: 1240, SEQ ID NO: 1242-SEQ ID NO: 1247, SEQ ID NO: 1254-SEQ ID NO: 1257; delivering the expression cassette to a cell of the subject; and expressing the small RNA payload in the cell, thereby treating the disease. 115. The method of embodiment 113 or embodiment 114, wherein the promoter sequence is SEQ ID NO: 376. 116. The method of embodiment 113 or embodiment 114, wherein the promoter sequence is SEQ ID NO: 1250. 117. The method of embodiment 115 or embodiment 116, wherein the transcription termination sequence is SEQ ID NO: 917. 118. The method of embodiment 115 or embodiment 116, wherein the transcription termination sequence is SEQ ID NO: 1254. 119. The method of embodiment 113 or embodiment 114, wherein the promoter sequence is SEQ ID NO: 168. 120. The method of embodiment 113 or embodiment 114, wherein the promoter sequence is SEQ ID NO: 1251. 121. The method of embodiment 113 or embodiment 114, wherein the transcription termination sequence is SEQ ID NO: 709. 122. The method of embodiment 120 or embodiment 121, wherein the transcription termination sequence is SEQ ID NO: 1255. 123. The method of embodiment 113 or embodiment 114, wherein the promoter sequence is SEQ ID NO: 1241. 124. The method of embodiment 123, wherein the transcription termination sequence is SEQ ID NO: 1242 or SEQ ID NO: 60. 125. The method of embodiment 113 or embodiment 114, wherein the promoter sequence is SEQ ID NO: 17. 126. The method of embodiment 125, wherein the transcription termination sequence is SEQ ID NO: 1242 or SEQ ID NO: 60. 127. A method of treating a disease in a subject, the method comprising: administering to the subject a composition comprising the expression cassette of any one of embodiments 1-79; delivering the expression cassette to a cell of the subject; and expressing a small RNA payload in the cell, thereby treating the disease. 128. The method of any one of embodiments 112-127, wherein the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease. 129. The method of any one of embodiments 112-128, wherein the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2). 130. The method of any one of embodiments 112-129, wherein the small RNA payload comprises an engineered guide RNA that hybridizes to a target sequence, and wherein the cell encodes the target sequence. 131. The method of embodiment 130, further comprising forming a guide-target RNA scaffold upon hybridization of the engineered guide RNA to the target sequence, recruiting an editing enzyme to the target sequence, and editing the target sequence with the editing enzyme. 132. The method of embodiment 130 or embodiment 131, wherein the target sequence comprises a mutation relative to a wild type sequence. 133. The method of embodiment 132, wherein editing the target sequence corrects the mutation in the target sequence. 134. The method of embodiment 132 or embodiment 133, wherein the mutation is a missense mutation. 135. The method of embodiment 132 or embodiment 133, wherein the mutation is a nonsense mutation. 136. The method of any one of embodiments 132-135, wherein the mutation is a G to A mutation. 137. The method of any one of embodiments 132-136, wherein the mutation is associated with the disease. 138. The method of any one of embodiments 132-137, wherein editing the target sequence comprises editing an untranslated region of the target. 139. The method of embodiment 138, wherein the untranslated region is a 5′ untranslated region or a 3′ untranslated region. 140. The method of embodiment 139, wherein the 3′ untranslated region is a polyadenylation sequence. 141. The method of any one of embodiments 131-140, wherein editing the target sequence comprises editing a translation initiation site. 142. The method of any one of embodiments 131-141, wherein editing the target sequence alters expression of the target sequence. 143. The method of embodiment 142, wherein editing the target sequence increases expression of the target sequence. 144. The method of embodiment 142, wherein editing the target sequence decreases expression of the target sequence. 145. The method of any one of embodiments 81-111 or 131-144, wherein the guide-target RNA scaffold comprises a structural feature. 146. The method of embodiment 145, wherein the structural feature is a bulge, a mismatch, an internal loop, a hairpin, or combinations thereof. 147. The method of embodiment 145, wherein the structural feature comprises the bulge, and wherein the bulge is a symmetric bulge. 148. The method of embodiment 145, wherein the structural feature comprises the bulge, and wherein the bulge is an asymmetric bulge. 149. The method of any one of embodiments 145-148, wherein the structural feature comprises the internal loop, and wherein the internal loop is a symmetric internal loop. 150. The method of any one of embodiments 145-149, wherein the structural feature comprises the internal loop, and wherein the internal loop is an asymmetric internal loop. 151. The method of any one of embodiments 145-150, wherein the structural feature comprises the hairpin, and wherein the hairpin is a recruitment hairpin or a non-recruitment hairpin. 152. The method of any one of embodiments 81-111 or 131-151, wherein the guide-target RNA scaffold comprises a Wobble base pair. 153. The method of any one of embodiments 81-111 or 131-152, wherein the editing enzyme comprises an ADAR, an APOBEC, or a Cas nuclease. 154. The method of embodiment 153, wherein the ADAR comprises ADAR1, ADAR2, ADAR3, or combinations thereof. 155. The method of any one of embodiments 81-111 or 131-154, wherein the target sequence comprises RNA or DNA. 156. The method of any one of embodiments 81-111 or 131-155, wherein the target sequence is a mRNA or a pre-mRNA. 157. The method of any one of embodiments 81-111 or 131-156, wherein editing the target sequence comprises deamidating a nucleotide of the target sequence. 158. The method of any one of embodiments 81-111 or 131-157, wherein the target sequence is edited with an efficiency of at least 10%, at least 20%, or at least 25%. 159. The method of any one of embodiments 81-158, wherein the expression cassette is delivered to the cell via a viral vector. 160. The method of embodiment 159, wherein the viral vector is an adenoviral vector, an adeno-associated viral vector, or a lentivector. 161. The method of embodiment 160, wherein the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, chimeras thereof, and combinations thereof. 162. A viral vector encapsidating the expression cassette of any one of embodiments 1-79. 163. The viral vector of embodiment 162, wherein the viral vector is an adeno-associated viral vector. 164. The viral vector of embodiment 163, wherein the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, chimeras thereof, and combinations thereof. 165. A pharmaceutical composition comprising the expression cassette of any one of embodiments 1-79 or the viral vector of any one of embodiments 162-164 and a pharmaceutically acceptable excipient, carrier, diluent, or combination thereof.

### EXAMPLES

The invention is further illustrated by the following non-limiting examples.

### Example 1

### Engineered Promoter Variants for Expression of Engineered Guide RNAs

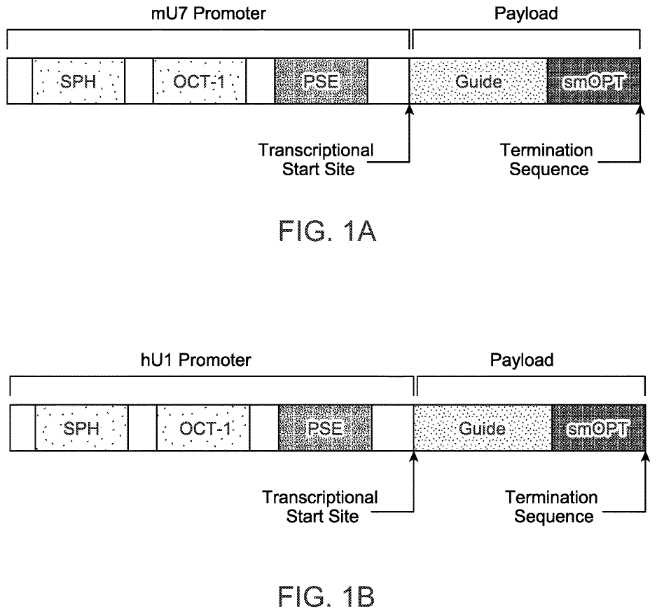

This example describes engineered promoter variants for expression of engineered guide RNAs, which are operably linked to small nuclear RNAs (snRNAs). Expression constructs were designed based on either a mouse U7 (mU7) promoter (FIG. 1A), such as SEQ ID NO: 15 (TAACAACATAGGAGCTGTGATTGGCTGTTTTCAGCCAATCAGCACTGACTCATTTG CATAGCCTTTACAAGCGGTCACAAACTCAAGAAACGAGCGGTTTTAATAGTCTTTTA GAATATTGTTTATCGAACCGAATAAGGAACTGTGCTTTGTGATTCACATATCAGTGG AGGGGTGTGGAAATGGCACCTTGATCTCACCCTCATCGAAAGTGGAGTTGATGTCC TTCCCTGGCTCGCTACAGACGCACTTCCGC), or a human U1 (hU1) promoter (FIG. 1B), such as SEQ ID NO: 13 (TAAGGACCAGCTTCTTTGGGAGAGAACAGACGCAGGGGCGGGAGGGAAAAAGGG AGAGGCAGACGTCACTTCCTCTTGGCGACTCTGGCAGCAGATTGGTCGGTTGAGTG GCAGAAAGGCAGACGGGGACTGGGCAAGGCACTGTCGGTGACATCACGGACAGGG CGACTTCTATGTAGATGAGGCAGCGCAGAGGCTGCTGCTTCGCCACTTGCTGCTTCG CCACGAAGGGAGTTCCCGTGCCCTGGGAGCGGGTTCAGGACCGCTGATCGGAAGTG AGAATCCCAGCTGTGTGTCAGGGCTGGAAAGGGCTCGGGAGTGCGCGGGGCAAGT GACCGTGTGTGTAAAGAGTGAGGCGTATGAGGCTGTGTCGGGGCAGAGCCCGAAG ATCTC). Elements of the mU7 and hU1 promoters, including the zinc finger 143 motif that binds a ZNF143 transcription factor, the OCT-1 transcription factor binding site, and the proximal sequence element (PSE) that recruits SNAPc and phosphorylated RNA polymerase II transcriptional machinery, were engineered to increase expression of downstream payload sequences, including engineered guide RNAs designed to hybridize to a target RNA and an Sm binding sequence (smOPT) that binds Sm proteins to form small nuclear ribonucleoprotein (snRNP) particles. Engineered guide RNAs form a guide-targeted RNA scaffold upon binding of the guide RNA to a target RNA, and thereby facilitate editing of a target adenosine (A) in the target RNA to inosine (I) by adenosine deaminase acting on RNA (ADAR). The smOPT facilitates nuclear trafficking of linked RNA sequences, including the engineered guide RNAs.

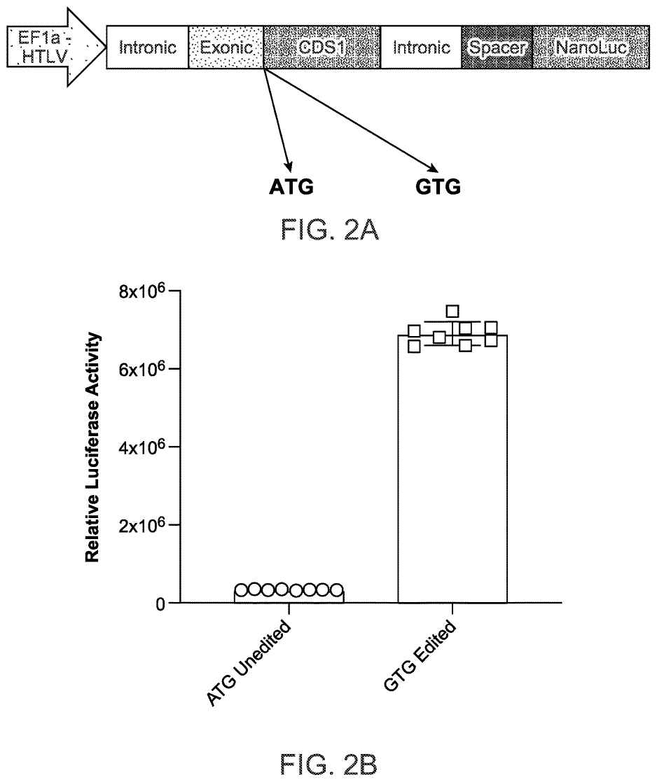

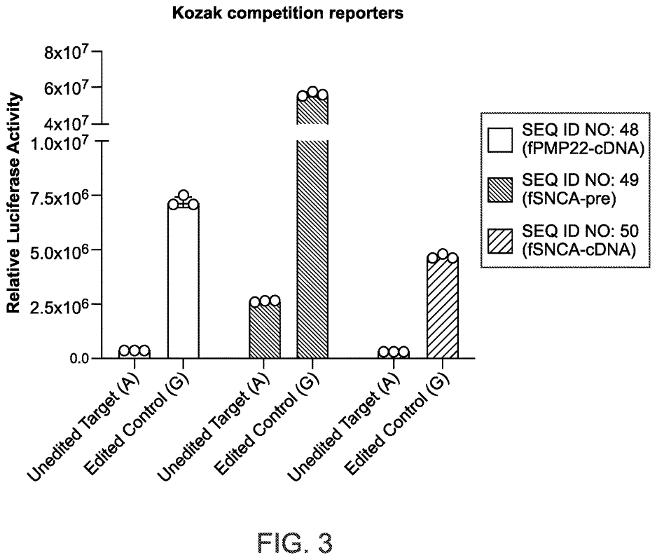

Constructs were screened for engineered guide RNA expression using a luciferase reporter assay. A Kozak competition reporter construct (FIG. 2A) containing an ATG initiation site that is deaminated to ITG, which is read as GTG, in the presence of expressed engineered guide RNA was used as an assay readout. In the absence of start codon deamination, the CDS1 was translated. Deamination of the start codon from ATG to ITG, facilitated by the expressed engineered guide RNA, disrupted CDS1 translation. Instead, a luciferase (“NanoLuc”) was translated. Luciferase activity was used as a readout of engineered guide RNA expression and engineered guide RNA-dependent editing. As shown in FIG. 2B, the unedited construct (“ATG unedited”) led to lower luciferase activity than the edited construct (“GTG Edited”). Reporter constructs of SEQ ID NO: 49 (CCAAGATGGATGGGAGATGCTAAATTTTTAATGCCAGAGCTAAGAATGTCTGCTTT GTCCAATGGTTAAATGAGTGTACACTTAAGAGAGTCTCACACTTTGGAGGGTTTCTC ATGATTTTTCAGTGTTTTTTGTTTATTTTTCCCCGAAAGTTCTCATTCAAAGTGTATTT TATGTTTTCCAGTGTGGTGTAAAGGAATTCATTAGCCATGGATGTATTCATGAAAGG ACTTTCAAAGGCCAAGGAGGGAGTTGTGGCTGCTGCTGAGAAGACCAAACAGGGTG TGGCAGAAGCAGCAGGAAAGACAAAGGAGGGTGTTCTCTATGTAGGTAGGGAAAC CCCAAATGTCAGTTTGGTGCTTGTTCATGAGAGATGGGTTAGGATAATCAATACTCT AAATGCTGGTAGTTCTCTCTCTGACTACAAGGACGACGACGACAAGT; “fSNCA-pre (ATG)”), and SEQ ID NO: 50 (GGGAGGAGCTTGCTTCTCCATTCTGGTGTGATCCAGGAACAGCTGTCTTCCAGCTCT GAATGTGGTGTAAAGGAATTCATTAGCCATGGATGTATTCATGAAAGGACTTTCAA AGGCCAAGGAGGGAGTTGTGGCTGCTGCTGAGAAGACCAAACAGGGTGTGGCAGA AGCAGCAGGAAAGACAAAGGAGGGTGTTCTCTATGTAGGCTCCAAGACCAAGGAG GGAGTGGTGCATGGTGTGGCAACAGTGGCTGAGAAGACCAAAGAGCAAGTGACAA ATGTTGGAGGAGCAGTGGTGACGGGTGTGACAGCAGTAGCCCAGAAGACAGTGGA GGGAGCAGGGAGCATTGCAGCAGCCACTGGCTTTGTCAAGAAGGACCAGTTGGGCA AG; “fSNCA-cDNA (ATG)”) were designed to test an engineered guide RNA targeting α-synuclein (SNCA), and a reporter construct of SEQ ID NO: 48 (AGTTACAGGGAGCACCACCAGGGAACATCTCGGGGAGCCTGGTTGGAAGCTGCAG GCTTAGTCTGTCGGCTGCGGGTCTCTGACTGCCCTGTGGGGAGGGTCTTGCCTTAAC ATCCCTTGCATTTGGCTGCAAAGAAATCTGCTTGGAAGAAGGGGTTACGCTGTTTGG CCGGGCAGAAACTCCGCTGAGCAGAACTTGCCGCCAGAGTGCTCCTCCTGTTGCTG AGTATCATCGTCCTCCACGTCGCGGTGCTGGTGCTGCTGTTCGTCTCCACGATCGTC AGCCAATGGATCGTGGGCAATGGACACGCAACTGATCTCTGGCAGAACTGTAGCAC CTCTTCCTCAGGAAATGTCCACCACTGTTTCTCATCATCACCAAACGAATGGCTGCA GTCTGTCCAGGCCACC; “fPMP22-cDNA (ATG)”) was designed to test an engineered guide RNA targeting PMP22. The PMP22 and SNCA reporters showed increased luciferase activity upon conversion of the ATG to GTG (FIG. 3).

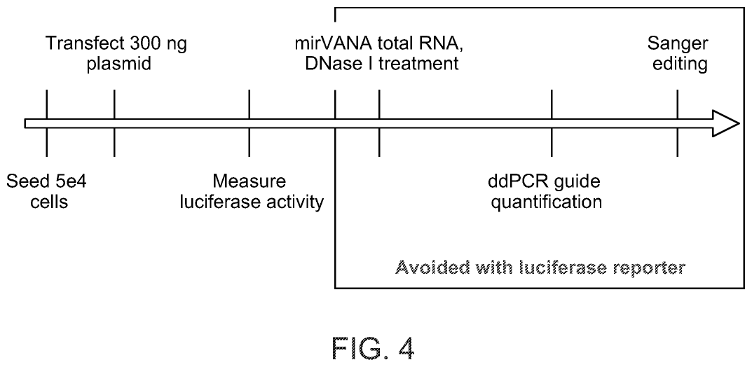

The workflow illustrated in FIG. 4 was used to screen engineered guide RNA constructs for guide RNA expression and editing. Cells were seeded at 5×10⁴ per 96-well, and transiently transfected with 300 ng of plasmid encoding an engineered guide RNA construct and a reporter construct. For luciferase reporter assays, luciferase activity was measured. Additional assays, including mirVANA total RNA isolation, DNaseI treatment, ddPCR guide quantification, and Sanger editing were performed for additional validation of the luciferase assay.

### Example 2

### Engineered Guide RNA Expression Constructs with Engineered OCT-1 Binding Sites

This example describes engineered guide RNA expression constructs with distal sequence elements (DSEs) comprising engineered OCT-1 binding sites. The OCT-1 binding site (SEQ ID NO: 21) of a mU7 promoter (SEQ ID NO: 15; TAACAACATAGGAGCTGTGATTGGCTGTTTTCAGCCAATCAGCACTGACTCATTTGC ATAGCCTTTACAAGCGGTCACAAACTCAAGAAACGAGCGGTTTTAATAGTCTTTTAG AATATTGTTTATCGAACCGAATAAGGAACTGTGCTTTGTGATTCACATATCAGTGGA GGGGTGTGGAAATGGCACCTTGATCTCACCCTCATCGAAAGTGGAGTTGATGTCCTT CCCTGGCTCGCTACAGACGCACTTCCGC) was replaced with various engineered OCT-1 binding sites, and expression of the SNCA-targeting guide RNA construct (SEQ ID NO: 1274; GACCGGCCACAACTCCCTCCTTGGCCTTTGAAAGTCCTTTCATGAATACATCCACGG CTAATGAATTCCTTTACACCACACTGGAAAACATAAAATACACTTTGAGTGGAATTT TTGGAGCAGGTTTTCTGACTTCGGTCGGAAAACCCCT) under control of the mU7 promoter was quantified using the luciferase reporter assay described in EXAMPLE 1. The effects of sequence changes as well as duplications were assayed. Duplicated sequences included two of the same OCT-1 binding sequence separated by an 8-nucleotide residue spacer. A random sequence (SEQ ID NO: 45) and a duplicated random sequence (SEQ ID NO: 46), were added in place of the OCT-1 binding sequence as a control that did not bind OCT-1 transcription factor. A construct encoding only a GFP cassette (“GFP ctrl”) was used as a negative control. Sequences of the tested OCT-1 binding sites are provided in TABLE 11.

TABLE 11
OCT-1 Binding Sequences

| SEQ ID NO: | Sequence |
|---|---|
| SEQ ID NO: 21 | ATTTGCAT |
| SEQ ID NO: 27 | ATGCAAAT |
| SEQ ID NO: 28 | ATGCAAATCAAGAGAAATGCAAAT |
| SEQ ID NO: 29 | ATGCATATTCAGCAAGAGAACTGCATATTCAT |
| SEQ ID NO: 30 | ATTTGCATCAAGAGAAATTTGCAT |
| SEQ ID NO: 45 | CGTAGTAC |
| SEQ ID NO: 46 | CGTAGTACCAAGAGAACGTAGTAC |

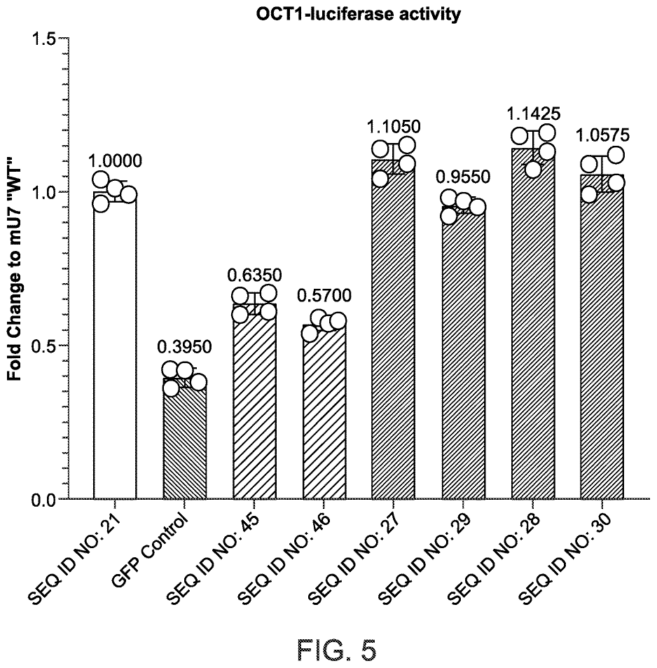

Fold change in luciferase activity relative to the original mU7 promoter sequence, which was used as a readout for guide RNA expression, was measured for each OCT-1 variant, as shown in FIG. 5. The construct with the OCT-1 binding site of SEQ ID NO: 28, corresponding to a duplicated OCT-1 binding site of SEQ ID NO: 27 showed the greatest increase in guide RNA expression relative to the original mU7 construct.

### Example 3

### Engineered Guide RNA Expression Constructs with Engineered Zinc Finger 143 Motifs

This example describes engineered guide RNA expression constructs with engineered zinc finger 143 motifs. The zinc finger 143 motif (SEQ ID NO: 20) of a mU7 promoter (SEQ ID NO: 15) was replaced with various engineered zinc finger 143 motifs, and expression of the SNCA-targeting guide RNA construct with an engineered mU7 termination sequence (SEQ ID NO: 1274) under control of the mU7 promoter was quantified using the luciferase reporter assay described in EXAMPLE 1. A random sequence (SEQ ID NO: 43) was added in place of the zinc finger 143 motif as a control that did not bind ZNF143 transcription factor. A construct encoding only a GFP cassette (“GFP ctrl”) was used as a negative control. Sequences of the tested zinc finger 143 motifs are provided in TABLE 12.

TABLE 12
Zinc Finger 143 Motifs

| SEQ ID NO: | Sequence |
|---|---|
| SEQ ID NO: 20 | GCCAATCAGCA |
| SEQ ID NO: 24 | ACTACAATTCCCAGC |
| SEQ ID NO: 25 | TTCCCAGCATGCCCCGCGC |
| SEQ ID NO: 26 | TACCCACAATGCCCTGC |
| SEQ ID NO: 43 | GTTACGCTTAGAATGGC |

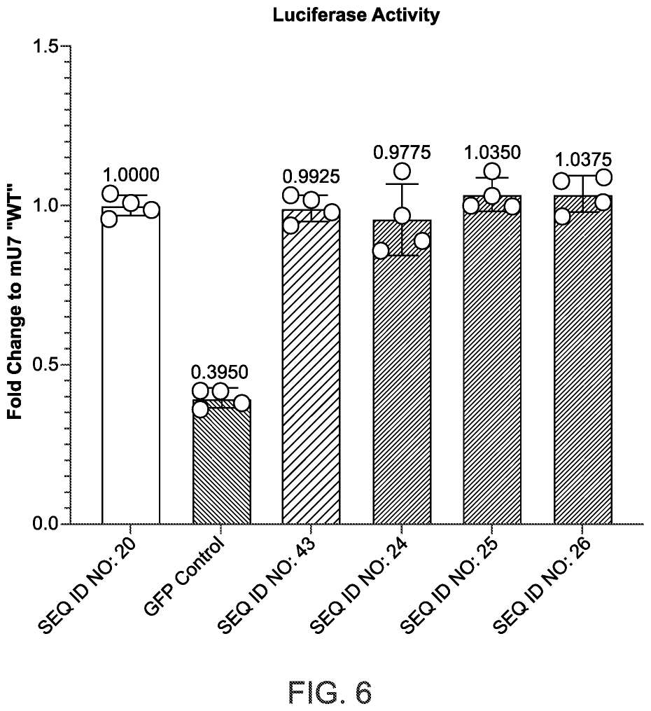

Fold change in luciferase activity relative to the original mU7 promoter sequence, which was used as a readout for guide RNA expression, was measured for each zinc finger 143 variant, as shown in FIG. 6. None of the zinc finger 143 variants showed a significant change in guide RNA expression relative to the original mU7 construct.

### Example 4

### Engineered Guide RNA Expression Constructs with Engineered Proximal Sequence Elements

This example describes engineered guide RNA expression constructs with engineered proximal sequence elements (PSEs). The PSE (SEQ ID NO: 22) of a mU7 promoter (SEQ ID NO: 15) was replaced with various PSEs, and expression of the SNCA-targeting guide RNA construct (SEQ ID NO: 1274) under control of the mU7 promoter was quantified using the luciferase reporter assay described in EXAMPLE 1. A random sequence (SEQ ID NO: 44) was added in place of the PSE as a control that did not recruit the transcription factor SNAPc. A construct encoding only a GFP cassette (“GFP ctrl”) was used as a negative control. Sequences of the tested PSEs are provided in TABLE 13.

TABLE 13
Proximal Sequence Elements

| SEQ ID NO: | Sequence |
|---|---|
| SEQ ID NO: 22 | CTCACCCTCATCGAAAGTGG |
| SEQ ID NO: 31 | AAGTCACCATGAGTGTAAAGGG |
| SEQ ID NO: 32 | AGGTCACCGTAACTATAAAAGA |
| SEQ ID NO: 33 | ACTTGACCTAAGTGTAAAGTT |
| SEQ ID NO: 34 | AAGTTACCATTACCCGTTTAGG |
| SEQ ID NO: 35 | AAATCACCATAAACGTGAAATG |
| SEQ ID NO: 36 | AAGTGACCTTGCGTGTAAAGGG |
| SEQ ID NO: 37 | AATGATCCTATATTTAGAGTGG |
| SEQ ID NO: 44 | CTGACAATGGCTACAGTCGA |

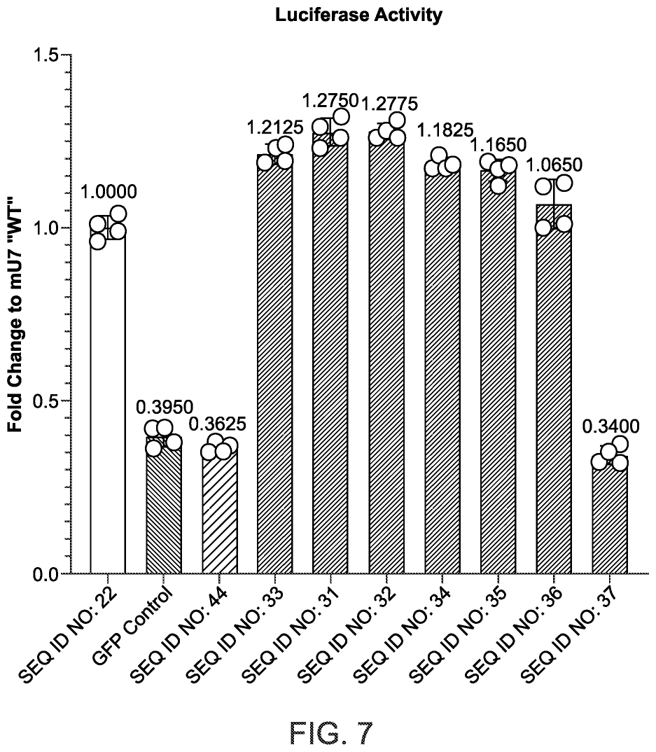

Fold change in luciferase activity relative to the original mU7 promoter sequence, which was used as a readout for guide RNA expression, was measured for each PSE variant, as shown in FIG. 7. The construct with the PSE of SEQ ID NO: 31 showed the greatest increase in guide RNA expression relative to the original mU7 construct.

### Example 5

### Engineered Guide RNA Expression Constructs with Engineered Transcription Termination Sequences

This example describes engineered guide RNA expression constructs with engineered 3′ box sequence elements. The 3′ box sequence element (SEQ ID NO: 23) of a mU7 promoter (SEQ ID NO: 15) was replaced with various engineered termination sequences, and expression of the SNCA-targeting guide RNA construct (SEQ ID NO: 1274) under control of the mU7 promoter was quantified using the luciferase reporter assay described in EXAMPLE 1. A random sequence (SEQ ID NO: 47) was added in place of the 3′ box sequence element as a control that lacked a termination sequence. A construct encoding only a GFP cassette (“GFP ctrl”) was used as a negative control. Sequences of the tested termination sequences are provided in TABLE 14.

TABLE 14
3′ Box Element Sequences

| SEQ ID NO: | Sequence |
|---|---|
| SEQ ID NO: 23 | GTCTACAATGAAAGC |
| SEQ ID NO: 40 | GTTTAATAAAAATAGA |
| SEQ ID NO: 41 | GTTTCAAAAACAGA |
| SEQ ID NO: 42 | GTTCAATGGCTGA |
| SEQ ID NO: 47 | ACTGGATTCAGTACGTACGTA |

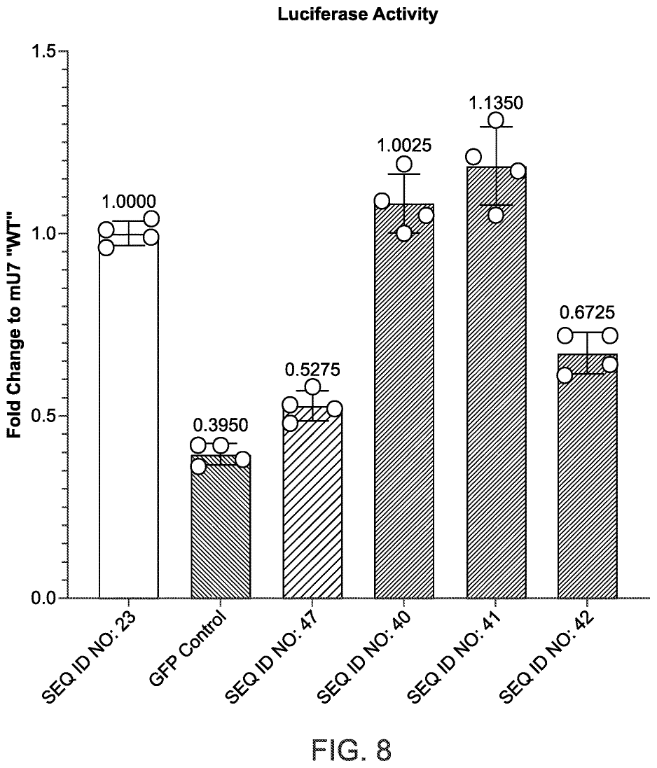

Fold change in luciferase activity relative to the original mU7 promoter sequence, which was used as a readout for guide RNA expression, was measured for each termination sequence variant, as shown in FIG. 8. The construct with the termination sequence of SEQ ID NO: 41 showed the greatest increase in guide RNA expression relative to the original mU7 construct.

### Example 6

### Combining Engineered Promoter Sequence Elements for Increased Engineered Guide RNA Expression

This example describes combining engineered promoter sequence elements for increased engineered guide RNA expression. The highest performing engineered promoter sequence elements identified in EXAMPLE 2-EXAMPLE 5 were tested in combination to identify combinations of elements that improved expression of either a PMP22-targeting engineered guide RNA (SEQ ID NO: 1273; GACCGCACCAGCACCGCGACGTGGAGGACGATGATACTCAGCAACAGGAGGAGCC CACTGGCGGCAAGTTCTGCTCAGCGGAGTTTCTGCCCGGCCAAACAGCGTGTGGAA TTTTTGGAGCAGGTTTTCTGACTTCGGTCGGAAAACCCCT) or an SNCA-targeting engineered guide RNA (e.g., SEQ ID NO: 1274 or SEQ ID NO: 1290). Constructs containing a distal sequence element (DSE) with a wild type zinc finger 143 motif of SEQ ID NO: 20, a wild type OCT-1 transcription factor binding sequence of SEQ ID NO: 21 or a variant OCT-1 transcription factor binding sequence of SEQ ID NO: 28, a wild type PSE of SEQ ID NO: 22 or a variant PSE of SEQ ID NO: 31, and a wild type 3′ box sequence element of SEQ ID NO: 23 or a variant3′ box sequence element of SEQ ID NO: 41. The screened expression cassettes encoding engineered guide RNA constructs are provided in TABLE 15.

TABLE 15
Engineered Guide RNA Expression Constructs with Engineered Promoter
Elements

| SEQ ID | | |
|---|---|---|
| NO: | Sequence | Target |
| SEQ ID | TAACAACATAGGAGCTGTGATTGGCTGTTTTCAGCCAATCAGCACTG | PMP22 |
| NO: 1 | ACTCATTTGCATAGCCTTTACAAGCGGTCACAAACTCAAGAAACGAG | |
| | CGGTTTTAATAGTCTTTTAGAATATTGTTTATCGAACCGAATAAGGAA | |
| | CTGTGCTTTGTGATTCACATATCAGTGGAGGGGTGTGGAAATGGCAC | |
| | CTTGATCTCACCCTCATCGAAAGTGGAGTTGATGTCCTTCCCTGGCTC | |
| | GCTACAGACGCACTTCCGCGACCGCACCAGCACCGCGACGTGGAGGA | |
| | CGATGATACTCAGCAACAGGAGGAGCCCACTGGCGGCAAGTTCTGCT | |
| | CAGCGGAGTTTCTGCCCGGCCAAACAGCGTGTGGAATTTTTGGAGCA | |
| | GGTTTTCTGACTTCGGTCGGAAAACCCCTCCCAATTTCACTGGTCTAC | |
| | AATGAAAGCAAAACAGTTCTCTTCCCCGCTCCCCGGTGTGTGAGAGG | |
| | GGCTTTGATCCTTCTCTGGTTTCCTAGGAAACGCGTATGTG | |
| SEQ ID | TAACAACATAGGAGCTGTGATTGGCTGTTTTCAGCCAATCAGCACTG | PMP22 |
| NO: 2 | ACTCATTTGCATAGCCTTTACAAGCGGTCACAAACTCAAGAAACGAG | |
| | CGGTTTTAATAGTCTTTTAGAATATTGTTTATCGAACCGAATAAGGAA | |
| | CTGTGCTTTGTGATTCACATATCAGTGGAGGGGTGTGGAAATGGCAC | |
| | CTTGATAAGTCACCATGAGTGTAAAGGGAGTTGATGTCCTTCCCTGGC | |
| | TCGCTACAGACGCACTTCCGCGACCGCACCAGCACCGCGACGTGGAG | |
| | GACGATGATACTCAGCAACAGGAGGAGCCCACTGGCGGCAAGTTCTG | |
| | CTCAGCGGAGTTTCTGCCCGGCCAAACAGCGTGTGGAATTTTTGGAG | |
| | CAGGTTTTCTGACTTCGGTCGGAAAACCCCTCCCAATTTCACTGGTCT | |
| | ACAATGAAAGCAAAACAGTTCTCTTCCCCGCTCCCCGGTGTGTGAGA | |
| | GGGGCTTTGATCCTTCTCTGGTTTCCTAGGAAACGCGTATGTG | |
| SEQ ID | TAACAACATAGGAGCTGTGATTGGCTGTTTTCAGCCAATCAGCACTG | PMP22 |
| NO: 3 | ACTCATTTGCATAGCCTTTACAAGCGGTCACAAACTCAAGAAACGAG | |
| | CGGTTTTAATAGTCTTTTAGAATATTGTTTATCGAACCGAATAAGGAA | |
| | CTGTGCTTTGTGATTCACATATCAGTGGAGGGGTGTGGAAATGGCAC | |
| | CTTGATCTCACCCTCATCGAAAGTGGAGTTGATGTCCTTCCCTGGCTC | |
| | GCTACAGACGCACTTCCGCGACCGCACCAGCACCGCGACGTGGAGGA | |
| | CGATGATACTCAGCAACAGGAGGAGCCCACTGGCGGCAAGTTCTGCT | |
| | CAGCGGAGTTTCTGCCCGGCCAAACAGCGTGTGGAATTTTTGGAGCA | |
| | GGTTTTCTGACTTCGGTCGGAAAACCCCTCCCAATTTCACTGGTTTCA | |
| | AAAACAGAAAAACAGTTCTCGTTTCAAAAACAGATTCCCCGCTCCCC | |
| | GGTGTGTGAGAGGGGCTTTGATCCTTCTCTGGTTTCCTAGGAAACGCG | |
| | TATGTG | |
| SEQ ID | TAACAACATAGGAGCTGTGATTGGCTGTTTTCAGCCAATCAGCACTG | PMP22 |
| NO: 4 | ACTCATTTGCATAGCCTTTACAAGCGGTCACAAACTCAAGAAACGAG | |
| | CGGTTTTAATAGTCTTTTAGAATATTGTTTATCGAACCGAATAAGGAA | |
| | CTGTGCTTTGTGATTCACATATCAGTGGAGGGGTGTGGAAATGGCAC | |
| | CTTGATAAGTCACCATGAGTGTAAAGGGAGTTGATGTCCTTCCCTGGC | |
| | TCGCTACAGACGCACTTCCGCGACCGCACCAGCACCGCGACGTGGAG | |
| | GACGATGATACTCAGCAACAGGAGGAGCCCACTGGCGGCAAGTTCTG | |
| | CTCAGCGGAGTTTCTGCCCGGCCAAACAGCGTGTGGAATTTTTGGAG | |
| | CAGGTTTTCTGACTTCGGTCGGAAAACCCCTCCCAATTTCACTGGTTT | |
| | CAAAAACAGAAAAACAGTTCTCTTCCCCGCTCCCCGGTGTGTGAGAG | |
| | GGGCTTTGATCCTTCTCTGGTTTCCTAGGAAACGCGTATGTG | |
| SEQ ID | TAACAACATAGGAGCTGTGATTGGCTGTTTTCAGCCAATCAGCACTG | PMP22 |
| NO: 5 | ACTCATGCAAATCAAGAGAAATGCAAATAGCCTTTACAAGCGGTCAC | |
| | AAACTCAAGAAACGAGCGGTTTTAATAGTCTTTTAGAATATTGTTTAT | |
| | CGAACCGAATAAGGAACTGTGCTTTGTGATTCACATATCAGTGGAGG | |
| | GGTGTGGAAATGGCACCTTGATAAGTCACCATGAGTGTAAAGGGAGT | |
| | TGATGTCCTTCCCTGGCTCGCTACAGACGCACTTCCGCGACCGCACCA | |
| | GCACCGCGACGTGGAGGACGATGATACTCAGCAACAGGAGGAGCCC | |
| | ACTGGCGGCAAGTTCTGCTCAGCGGAGTTTCTGCCCGGCCAAACAGC | |
| | GTGTGGAATTTTTGGAGCAGGTTTTCTGACTTCGGTCGGAAAACCCCT | |
| | CCCAATTTCACTGGTTTCAAAAACAGAAAAACAGTTCTCTTCCCCGCT | |
| | CCCCGGTGTGTGAGAGGGGCTTTGATCCTTCTCTGGTTTCCTAGGAAA | |
| | CGCGTATGTG | |
| SEQ ID | TAACAACATAGGAGCTGTGATTGGCTGTTTTCAGCCAATCAGCACTG | SNCA |
| NO: 6 | ACTCATTTGCATAGCCTTTACAAGCGGTCACAAACTCAAGAAACGAG | |
| | CGGTTTTAATAGTCTTTTAGAATATTGTTTATCGAACCGAATAAGGAA | |
| | CTGTGCTTTGTGATTCACATATCAGTGGAGGGGTGTGGAAATGGCAC | |
| | CTTGATCTCACCCTCATCGAAAGTGGAGTTGATGTCCTTCCCTGGCTC | |
| | GCTACAGACGCACTTCCGCGACCGGCCACAACTCCCTCCTTGGCCTTT | |
| | GAAAGTCCTTTCATGAATACATCCACGGCTAATGAATTCCTTTACACC | |
| | ACACTGGAAAACATAAAATACACTTTGAGTGGAATTTTTGGAGCAGG | |
| | TTTTCTGACTTCGGTCGGAAAACCCCTCCCAATTTCACTGGTCTACAA | |
| | TGAAAGCAAAACAGTTCTCTTCCCCGCTCCCCGGTGTGTGAGAGGGG | |
| | CTTTGATCCTTCTCTGGTTTCCTAGGAAACGCGTATGTG | |
| SEQ ID | TAAGGACCAGCTTCTTTGGGAGAGAACAGACGCAGGGGGGGGAGGG | SNCA |
| NO: 7 | AAAAAGGGAGAGGCAGACGTCACTTCCTCTTGGCGACTCTGGCAGCA | |
| | GATTGGTCGGTTGAGTGGCAGAAAGGCAGACGGGGACTGGGCAAGG | |
| | CACTGTCGGTGACATCACGGACAGGGCGACTTCTATGTAGATGAGGC | |
| | AGCGCAGAGGCTGCTGCTTCGCCACTTGCTGCTTCGCCACGAAGGGA | |
| | GTTCCCGTGCCCTGGGAGCGGGTTCAGGACCGCTGATCGGAAGTGAG | |
| | AATCCCAGCTGTGTGTCAGGGCTGGAAAGGGCTCGGGAGTGCGCGGG | |
| | GCAAGTGACCGTGTGTGTAAAGAGTGAGGCGTATGAGGCTGTGTCGG | |
| | GGCAGAGCCCGAAGATCTCACCGGCCACAACTCCCTCCTTGGCCTTT | |
| | GAAAGTCCTTTCATGAATACATCCACGGCTAATGAATTCCTTTACACC | |
| | ACACTGGAAAACATAAAATACACTTTGAGTGGAATTTTTGGAGCAGG | |
| | TTTTCTGACTTCGGTCGGAAAACCCCTCCCAATTTCACTGGTCTACAA | |
| | TGAAAGCAAAACAGTTCTCTTCCCCGCTCCCCGGTGTGTGAGAGGGG | |
| | CTTTGATCCTTCTCTGGTTTCCTAGGAAACGCGTATGTG | |
| SEQ ID | TTAACAACAACGAAGGGGCTGTGACTGGCTGCTTTCTCAACCAATCA | SNCA |
| NO: 8 | GCACCGAACTCATTTGCATGGGCTGAGAACAAATGTTCGCGAACTCT | |
| | AGAAATGAATGACTTAAGTAAGTTCCTTAGAATATTATTTTTCCTACT | |
| | GAAAGTTACCACATGCGTCGTTGTTTATACAGTAATAGGAACAAGAA | |
| | AAAAGTCACCTAAGCTCACCCTCATCAATTGTGGAGTTCCTTTATATC | |
| | CCATCTTCTCTCCAAACACATACGCAGACCGGCCACAACTCCCTCCTT | |
| | GGCCTTTGAAAGTCCTTTCATGAATACATCCACGGCTAATGAATTCCT | |
| | TTACACCACACTGGAAAACATAAAATACACTTTGAGTGGAATTTTTG | |
| | GAGCAGGTTTTCTGACTTCGGTCGGAAAACCCCTCCCAATTTCACTGG | |
| | TCTACAATGAAAGCAAAACAGTTCTCTTCCCCGCTCCCCGGTGTGTGA | |
| | GAGGGGCTTTGATCCTTCTCTGGTTTCCTAGGAAACGCGTATGTG | |
| SEQ ID | TAACAACATAGGAGCTGTGATTGGCTGTTTTCAGCCAATCAGCACTG | SNCA |
| NO: 9 | ACTCATTTGCATAGCCTTTACAAGCGGTCACAAACTCAAGAAACGAG | |
| | CGGTTTTAATAGTCTTTTAGAATATTGTTTATCGAACCGAATAAGGAA | |
| | CTGTGCTTTGTGATTCACATATCAGTGGAGGGGTGTGGAAATGGCAC | |
| | CTTGATAAGTCACCATGAGTGTAAAGGGAGTTGATGTCCTTCCCTGGC | |
| | TCGCTACAGACGCACTTCCGCGACCGGCCACAACTCCCTCCTTGGCCT | |
| | TTGAAAGTCCTTTCATGAATACATCCACGGCTAATGAATTCCTTTACA | |
| | CCACACTGGAAAACATAAAATACACTTTGAGTGGAATTTTTGGAGCA | |
| | GGTTTTCTGACTTCGGTCGGAAAACCCCTCCCAATTTCACTGGTCTAC | |
| | AATGAAAGCAAAACAGTTCTCTTCCCCGCTCCCCGGTGTGTGAGAGG | |
| | GGCTTTGATCCTTCTCTGGTTTCCTAGGAAACGCGTATGTG | |
| SEQ ID | TAACAACATAGGAGCTGTGATTGGCTGTTTTCAGCCAATCAGCACTG | SNCA |
| NO: 10 | ACTCATTTGCATAGCCTTTACAAGCGGTCACAAACTCAAGAAACGAG | |
| | CGGTTTTAATAGTCTTTTAGAATATTGTTTATCGAACCGAATAAGGAA | |
| | CTGTGCTTTGTGATTCACATATCAGTGGAGGGGTGTGGAAATGGCAC | |
| | CTTGATCTCACCCTCATCGAAAGTGGAGTTGATGTCCTTCCCTGGCTC | |
| | GCTACAGACGCACTTCCGCGACCGGCCACAACTCCCTCCTTGGCCTTT | |
| | GAAAGTCCTTTCATGAATACATCCACGGCTAATGAATTCCTTTACACC | |
| | ACACTGGAAAACATAAAATACACTTTGAGTGGAATTTTTGGAGCAGG | |
| | TTTTCTGACTTCGGTCGGAAAACCCCTCCCAATTTCACTGGTTTCAAA | |
| | AACAGAAAAACAGTTCTCGTTTCAAAAACAGATTCCCCGCTCCCCGG | |
| | TGTGTGAGAGGGGCTTTGATCCTTCTCTGGTTTCCTAGGAAACGCGTA | |
| | TGTG | |
| SEQ ID | TAACAACATAGGAGCTGTGATTGGCTGTTTTCAGCCAATCAGCACTG | SNCA |
| NO: 11 | ACTCATTTGCATAGCCTTTACAAGCGGTCACAAACTCAAGAAACGAG | |
| | CGGTTTTAATAGTCTTTTAGAATATTGTTTATCGAACCGAATAAGGAA | |
| | CTGTGCTTTGTGATTCACATATCAGTGGAGGGGTGTGGAAATGGCAC | |
| | CTTGATAAGTCACCATGAGTGTAAAGGGAGTTGATGTCCTTCCCTGGC | |
| | TCGCTACAGACGCACTTCCGCGACCGGCCACAACTCCCTCCTTGGCCT | |
| | TTGAAAGTCCTTTCATGAATACATCCACGGCTAATGAATTCCTTTACA | |
| | CCACACTGGAAAACATAAAATACACTTTGAGTGGAATTTTTGGAGCA | |
| | GGTTTTCTGACTTCGGTCGGAAAACCCCTCCCAATTTCACTGGTTTCA | |
| | AAAACAGAAAAACAGTTCTCTTCCCCGCTCCCCGGTGTGTGAGAGGG | |
| | GCTTTGATCCTTCTCTGGTTTCCTAGGAAACGCGTATGTG | |
| SEQ ID | TAACAACATAGGAGCTGTGATTGGCTGTTTTCAGCCAATCAGCACTG | SNCA |
| NO: 12 | ACTCATGCAAATCAAGAGAAATGCAAATAGCCTTTACAAGCGGTCAC | |
| | AAACTCAAGAAACGAGCGGTTTTAATAGTCTTTTAGAATATTGTTTAT | |
| | CGAACCGAATAAGGAACTGTGCTTTGTGATTCACATATCAGTGGAGG | |
| | GGTGTGGAAATGGCACCTTGATAAGTCACCATGAGTGTAAAGGGAGT | |
| | TGATGTCCTTCCCTGGCTCGCTACAGACGCACTTCCGCGACCGGCCAC | |
| | AACTCCCTCCTTGGCCTTTGAAAGTCCTTTCATGAATACATCCACGGC | |
| | TAATGAATTCCTTTACACCACACTGGAAAACATAAAATACACTTTGA | |
| | GTGGAATTTTTGGAGCAGGTTTTCTGACTTCGGTCGGAAAACCCCTCC | |
| | CAATTTCACTGGTTTCAAAAACAGAAAAACAGTTCTCTTCCCCGCTCC | |
| | CCGGTGTGTGAGAGGGGCTTTGATCCTTCTCTGGTTTCCTAGGAAACG | |
| | CGTATGTG | |

The PMP22-targeting guide RNA expression construct of SEQ ID NO: 1 included a wild type zinc finger 143 motif of SEQ ID NO: 20, a wild type OCT-1 transcription factor binding sequence of SEQ ID NO: 21, a wild type PSE of SEQ ID NO: 22, and a wild type 3′ box sequence element of SEQ ID NO: 23. The PMP22-targeting guide RNA expression construct of SEQ ID NO: 2 included a wild type zinc finger 143 motif of SEQ ID NO: 20, a wild type OCT-1 transcription factor binding sequence of SEQ ID NO: 21, a variant PSE of SEQ ID NO: 31, and a wild type 3′ box sequence element of SEQ ID NO: 23. The PMP22-targeting guide RNA expression construct of SEQ ID NO: 3 included a wild type zinc finger 143 motif of SEQ ID NO: 20, a wild type OCT-1 transcription factor binding sequence of SEQ ID NO: 21, a wild type PSE of SEQ ID NO: 22, and two instances of a variant 3′ box sequence element of SEQ ID NO: 41. The PMP22-targeting guide RNA expression construct of SEQ ID NO: 4 included a wild type zinc finger 143 motif of SEQ ID NO: 20, a wild type OCT-1 transcription factor binding sequence of SEQ ID NO: 21, a variant PSE of SEQ ID NO: 31, and a variant 3′ box sequence element of SEQ ID NO: 41. The PMP22-targeting guide RNA expression construct of SEQ ID NO: 5 included a wild type zinc finger 143 motif of SEQ ID NO: 20, a variant OCT-1 transcription factor binding sequence of SEQ ID NO: 28, a variant PSE of SEQ ID NO: 31, and a variant 3′ box sequence element of SEQ ID NO: 41.

The SNCA-targeting guide RNA expression construct of SEQ ID NO: 6 included a wild type zinc finger 143 motif of SEQ ID NO: 20, a wild type OCT-1 transcription factor binding sequence of SEQ ID NO: 21, a wild type PSE of SEQ ID NO: 22, and a wild type 3′ box sequence element of SEQ ID NO: 23. The SNCA-targeting guide RNA expression construct of SEQ ID NO: 9 included a wild type zinc finger 143 motif of SEQ ID NO: 20, a wild type OCT-1 transcription factor binding sequence of SEQ ID NO: 21, a variant PSE of SEQ ID NO: 31, and a wild type 3′ box sequence element of SEQ ID NO: 23. The SNCA-targeting guide RNA expression construct of SEQ ID NO: 10 included a wild type zinc finger 143 motif of SEQ ID NO: 20, a wild type OCT-1 transcription factor binding sequence of SEQ ID NO: 21, a wild type PSE of SEQ ID NO: 22, and two instances of a variant 3′ box sequence element of SEQ ID NO: 41. The SNCA-targeting guide RNA expression construct of SEQ ID NO: 11 included a wild type zinc finger 143 motif of SEQ ID NO: 20, a wild type OCT-1 transcription factor binding sequence of SEQ ID NO: 21, a variant PSE of SEQ ID NO: 31, and a variant 3′ box sequence element of SEQ ID NO: 41. The SNCA-targeting guide RNA expression construct of SEQ ID NO: 12 included a wild type zinc finger 143 motif of SEQ ID NO: 20, a variant OCT-1 transcription factor binding sequence of SEQ ID NO: 28, a variant PSE of SEQ ID NO: 31, and a variant 3′ box sequence element of SEQ ID NO: 41.

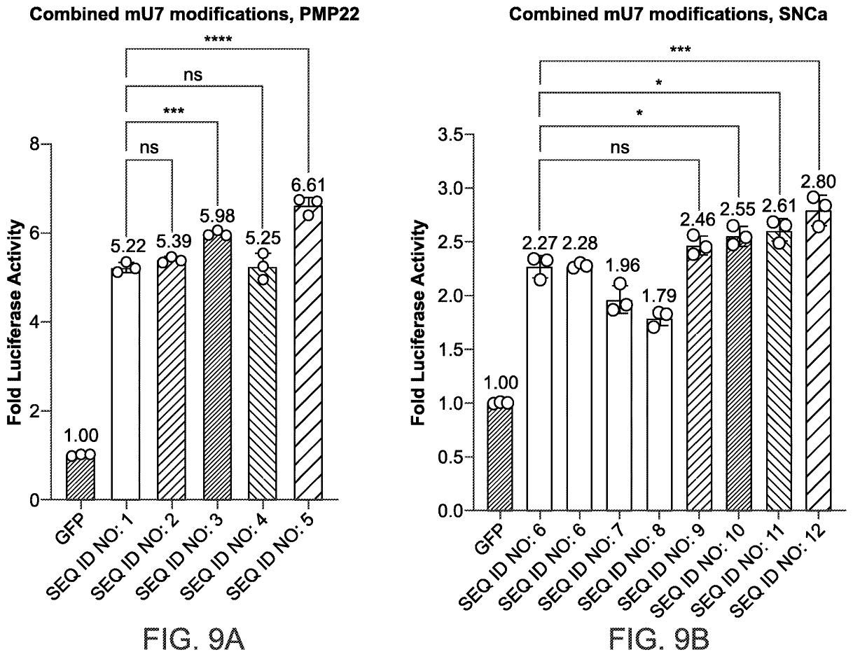

Constructs expressing either the PMP22-targeting engineered guide RNA (FIG. 9A) or the SNCA-targeting engineered guide RNA (FIG. 9B) were screened using the luciferase assay described in EXAMPLE 1. Expression of the SNCA-targeting guide RNA was also tested under control of a human U1 promoter (SEQ ID NO: 13) and a human U7 promoter (SEQ ID NO: 14; TTAACAACAACGAAGGGGCTGTGACTGGCTGCTTTCTCAACCAATCAGCACCGAAC TCATTTGCATGGGCTGAGAACAAATGTTCGCGAACTCTAGAAATGAATGACTTAAG TAAGTTCCTTAGAATATTATTTTTCCTACTGAAAGTTACCACATGCGTCGTTGTTTAT ACAGTAATAGGAACAAGAAAAAAGTCACCTAAGCTCACCCTCATCAATTGTGGAGT TCCTTTATATCCCATCTTCTCTCCAAACACATACGCA). A construct encoding only a GFP cassette (“GFP ctrl”) was used as a negative control.

For the PMP22-targeting engineered guide RNA, the construct with the wild type zinc finger 143 motif, the variant OCT-1 transcription factor binding sequence, the variant PSE, and the variant 3′ box sequence element (SEQ ID NO: 5) showed the greatest luciferase activity, indicative of highest guide RNA expression and RNA editing. For the SNCA-targeting engineered guide RNA, the construct with the wild type zinc finger 143 motif, the variant OCT-1 transcription factor binding sequence, the variant PSE, and the variant 3′ box sequence element (SEQ ID NO: 12) showed the greatest luciferase activity, indicative of highest guide RNA expression and RNA editing. These data suggest that the engineered promoter sequences of SEQ ID NO: 17 and SEQ ID NO: 16 effectively enhanced transcription of a payload sequence.

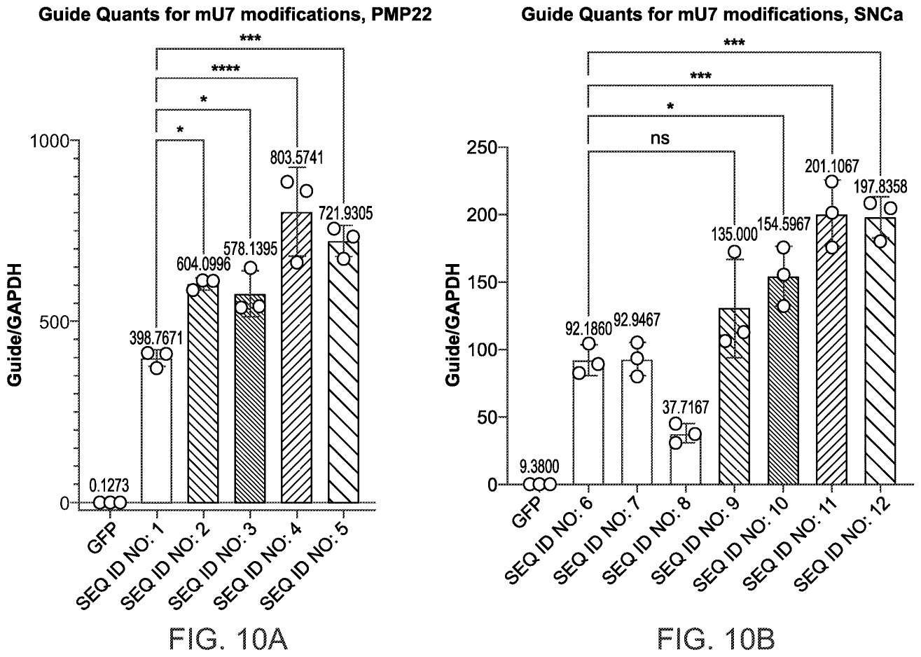

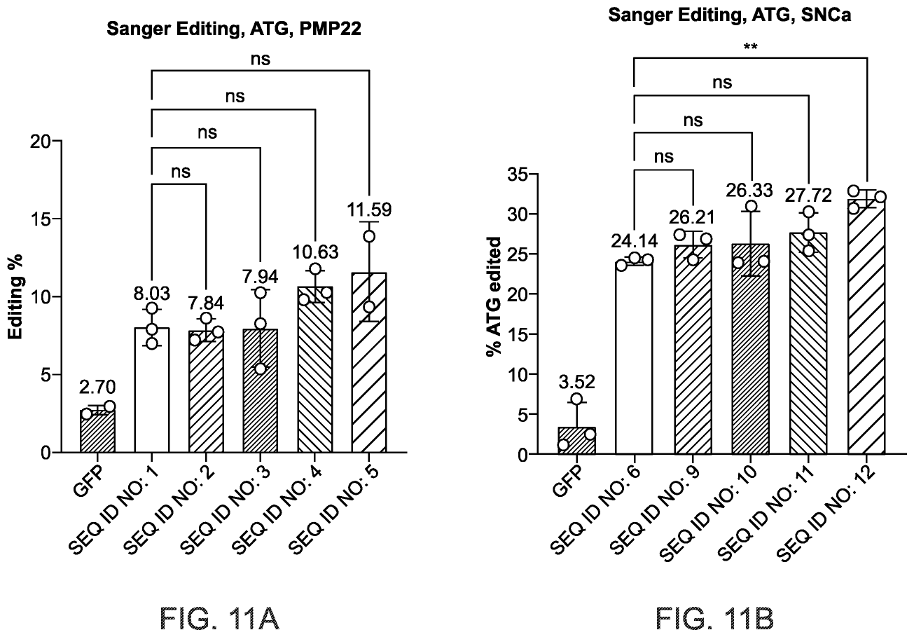

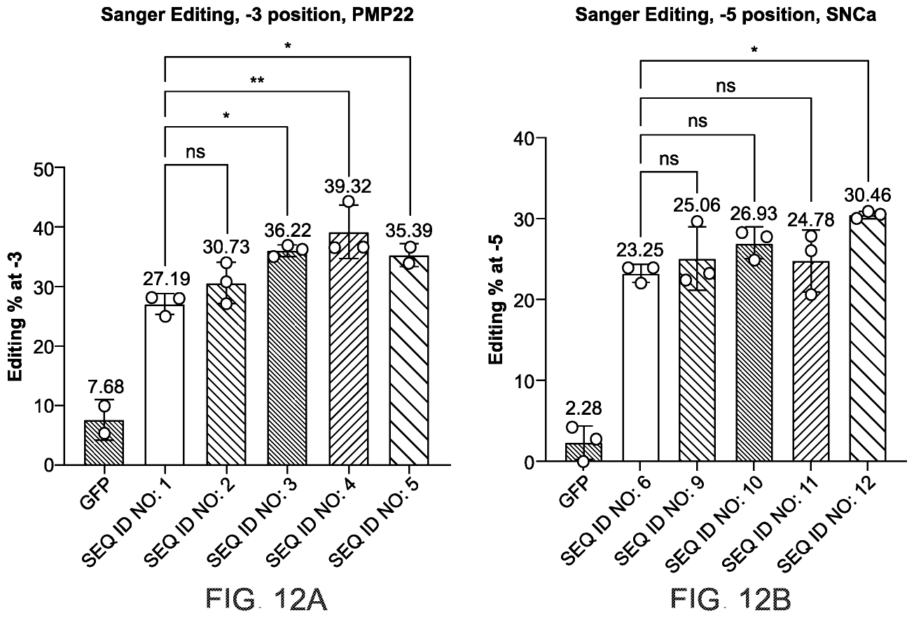

The results of the luciferase assay were verified using guide quantification (FIG. 10A and FIG. 10B for PMP22 and SNCA, respectively), Sanger editing of ATG (FIG. 11A and FIG. 11B for PMP22 and SNCA, respectively), and Sanger editing of the −3 or −5 position (FIG. 12A and FIG. 12B for PMP22 and SNCA, respectively). As measured by guide quantification and Sanger editing of the −3 position, the PMP22-targeting engineered guide RNA construct with the wild type zinc finger 143 motif, the wild OCT-1 transcription factor binding sequence, the variant PSE, and the variant 3′ box sequence element (SEQ ID NO: 4) showed the most guide RNA expression. As measured by Sanger editing of ATG, the PMP22-targeting engineered guide RNA construct with the wild type zinc finger 143 motif, the variant OCT-1 transcription factor binding sequence, the variant PSE, and the variant 3′ box sequence element (SEQ ID NO: 5) showed the most guide RNA expression. As measured by guide quantification, the SNCA-targeting engineered guide RNA construct with the wild type zinc finger 143 motif, the wild OCT-1 transcription factor binding sequence, the variant PSE, and the variant 3′ box sequence element (SEQ ID NO: 11) showed the most guide RNA expression. As measured by Sanger editing of ATG and Sanger editing of the −5 position, the SNCA-targeting engineered guide RNA construct with the wild type zinc finger 143 motif, the variant OCT-1 transcription factor binding sequence, the variant PSE, and the variant 3′ box sequence element (SEQ ID NO: 12) showed the most guide RNA expression.

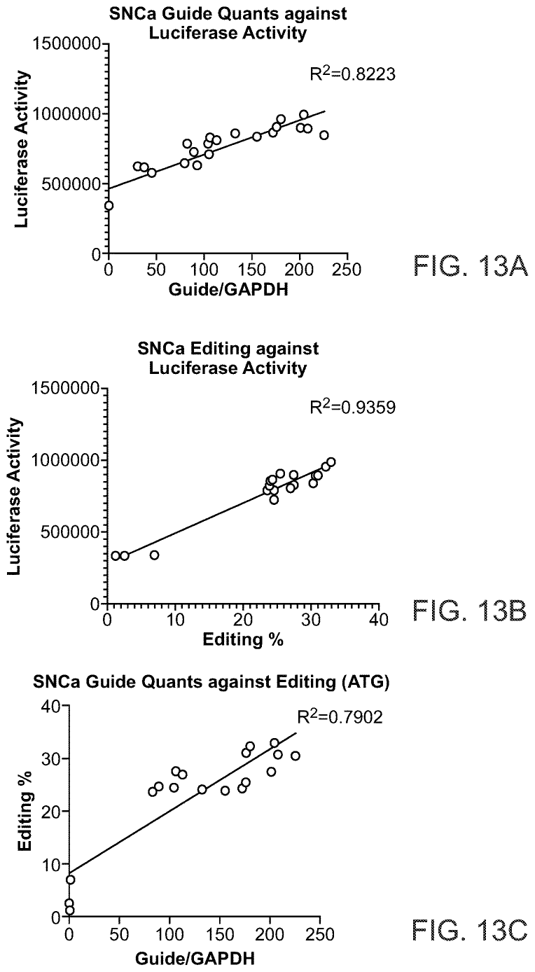

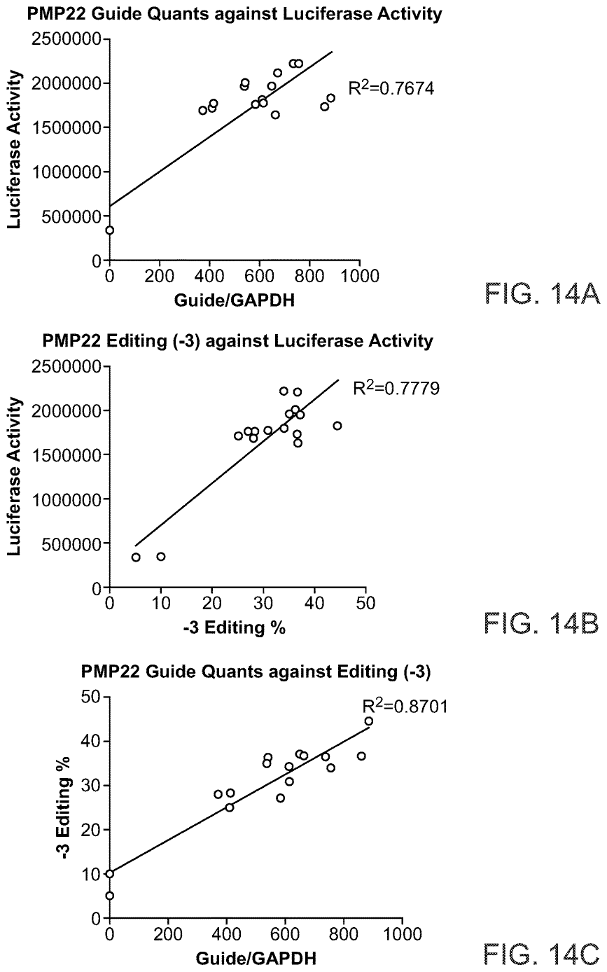

The different quantification methods, luciferase activity, Sanger editing, and guide quantification, were compared by linear regression. As seen in FIG. 13A-FIG. 13C for SNCA-targeting constructs, assay results measured by guide quantification and luciferase activity (FIG. 13A), Sanger editing of ATG and luciferase activity (FIG. 13B), and guide quantification and Sanger editing of ATG (FIG. 13C) were well correlated. As seen in FIG. 14A-FIG. 14C for PMP22-targeting constructs, assay results measured by guide quantification and luciferase activity (FIG. 14A), Sanger editing of the −3 position and luciferase activity (FIG. 14B), and guide quantification and Sanger editing of the −3 position (FIG. 14C) were well correlated.

### Example 7

### Single Copy Integration of Engineered Guide RNA Expression Constructs

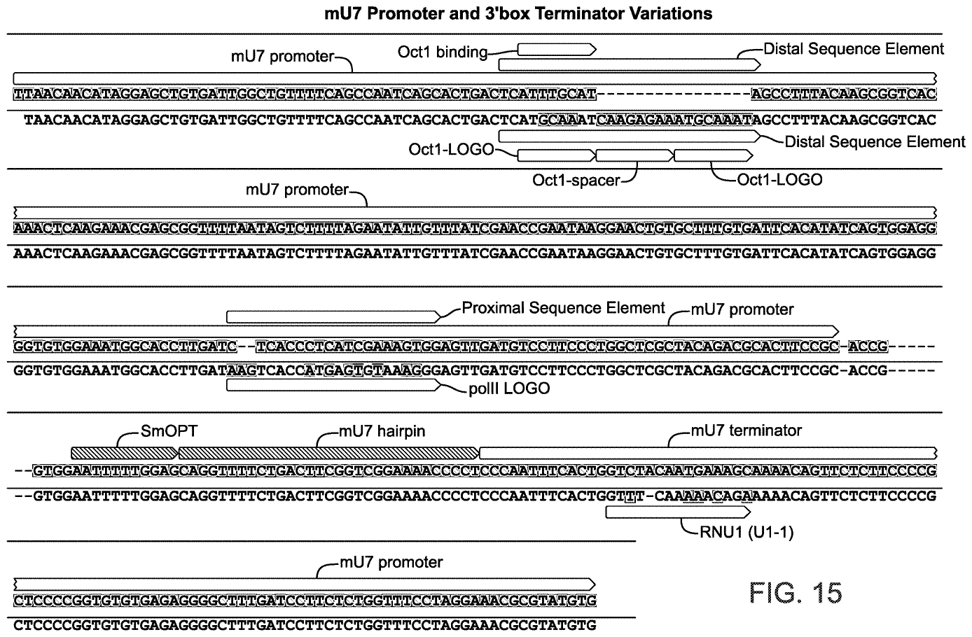

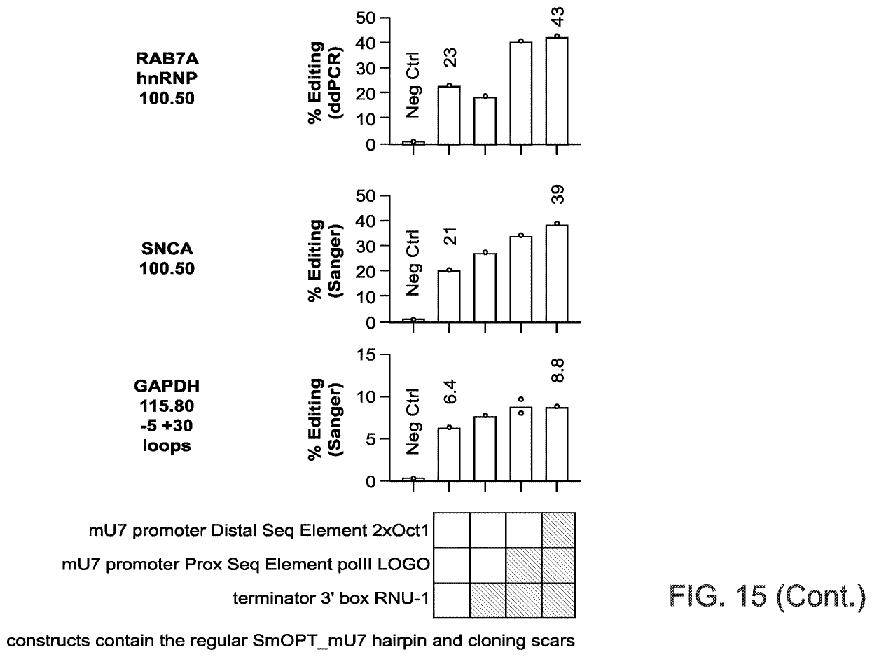

This example describes single copy integration of engineered guide RNA expression constructs. A single copy of each promoter variant was inserted into the genome of a HEK293T (FIG. 15, left) cell by plasmid transfection and single copy integration. The cells were enriched by Puromycin selection and thirteen days post transfection RNA was isolated, cDNA was generated and editing of different targets was assessed by ddPCR or Sanger sequencing. As shown in FIG. 15, the engineered promoters facilitated single copy integration of an engineered guide RNA targeting RAB7A (top right), GAPDH (middle right), and SNCA (bottom right). The results demonstrated that editing rates doubled when using an engineered promoter of SEQ ID NO: 17 and an engineered termination sequence of SEQ ID NO: 60 (which includes a 3′ box sequence element of SEQ ID NO: 41), as compared to a wild type mU7 promoter of SEQ ID NO: 15.

Single copy integration was used to control for copy number to evaluate the effect of engineered promoters on payload expression.

### Example 8

### Expression of Engineered Guide RNAs in Different Cell Types

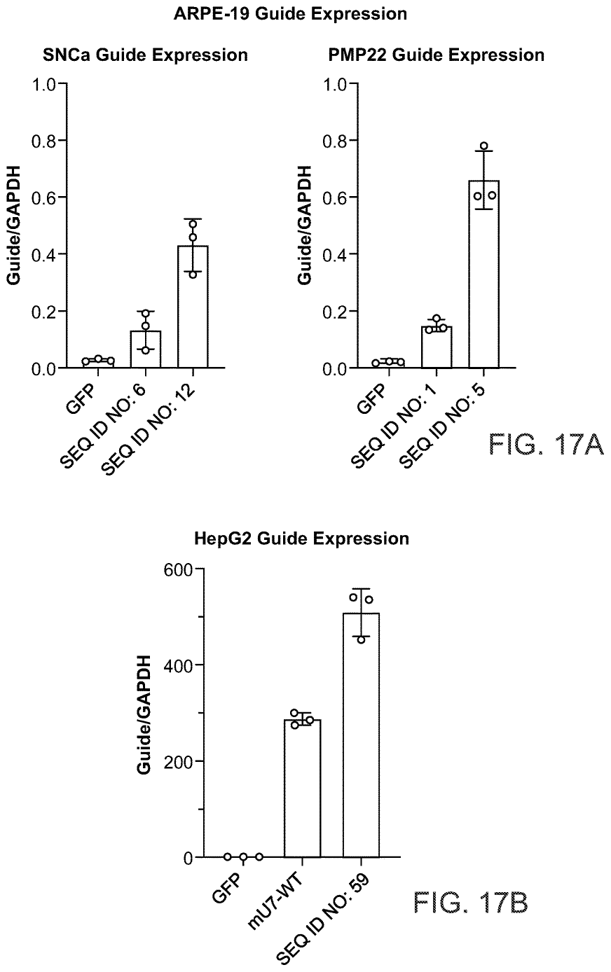

This example describes expression of engineered guide RNAs in different cell types using an engineered expression cassette. In a first assay, engineered guide RNAs targeting either SNCA or PMP22 (SEQ ID NO: 1274 and SEQ ID NO: 1273, respectively) were inserted into either a wild type mouse U7 expression cassette or an engineered mouse U7 expression cassette and expressed in ARPE-19 cells (FIG. 17A). The SNCA and PMP22 wild type mouse expression cassettes had sequences of SEQ ID NO: 6 and SEQ ID NO: 1, respectively. The SNCA and PMP22 engineered mouse U7 expression cassettes had sequences of SEQ ID NO: 12 and SEQ ID NO: 5, respectively. The engineered mouse U7 expression cassettes of SEQ ID NO: 12 and SEQ ID NO: 5 each contained an engineered promoter of SEQ ID NO: 17, comprising an OCT-1 transcription factor binding sequence of SEQ ID NO: 28 and a PSE of SEQ ID NO: 31, and an engineered termination sequence of SEQ ID NO: 60, comprising a transcription termination sequence of SEQ ID NO: 41. As shown in FIG. 17A, the engineered mouse U7 expression cassettes (SEQ ID NO: 12 or SEQ ID NO: 5) enhanced expression of the SNCA-targeting guide RNA and PMP22-targeting guide RNA in ARPE-19 cells relative to the corresponding wild type mouse U7 expression cassettes (SEQ ID NO: 6 or SEQ ID NO: 1).

In a second assay, an engineered guide RNA targeting SERPINA1 (SEQ ID NO: 61; GACCGTAGACATGGGTATGGCCTCTAATTTGTAGGCCCCAGCAGCTTCAGTCCCTTA CTCGTCGTACCAGAGCACAGCCAGTCGTATGCACGGCGTGGAATTTTTGGAGCAGG TTTTCTGACTTCGGTCGGAAAACCCCT) was inserted into either a wild type mouse U7 expression cassette or an engineered mouse U7 expression cassette and expressed in HepG2 cells (FIG. 17B). The SERPINA1 engineered mouse U7 expression cassettes had a sequence of SEQ ID NO: 59. The engineered mouse U7 expression cassette of SEQ ID NO: 59 contained an engineered promoter of SEQ ID NO: 16, comprising a PSE of SEQ ID NO: 31, and an engineered termination sequence of SEQ ID NO: 60, comprising a transcription termination sequence of SEQ ID NO: 41. As shown in FIG. 17B, the engineered mouse U7 expression cassette (SEQ ID NO: 59) enhanced expression of the SERPINA1-targeting guide RNA in HepG2 cells relative to the corresponding wild type mouse U7 expression cassette.

Together, these data demonstrate that engineered expression cassettes comprising engineered promoters of SEQ ID NO: 16 or SEQ ID NO: 17 and engineered termination sequences of SEQ ID NO: 60 enhance RNA payload expression in multiple different cell types, including ARPE-19 cells and HepG2 cells.

### Example 9

### Modified U7 Promoters Provide Exon Skipping

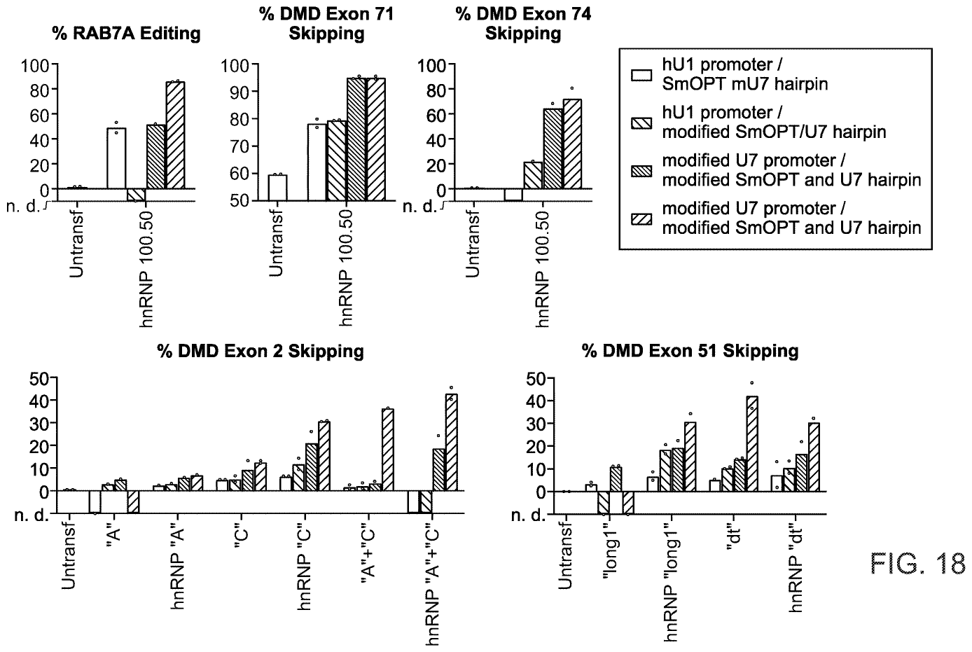

The modified U7 promoter of SEQ ID NO: 17 and SEQ ID NO: 60 flanking the guide RNA-SmOPT or ASO on the 5′ and 3′ ends respectively was tested in human RD rhabdomyosarcoma cells (CCL-136). FIG. 18 shows the percent of RAB7A editing or DMD exon skipping by the indicated engineered guide RNA. RD cells were transfected with plasmid constructs expressing the antisense guide RNA from a human U1 promoter (SEQ ID NO: 13) or a modified U7 promoter (SEQ ID NO: 17) and a termination sequence of SEQ ID NO: 60, along with a plasmid expressing piggybac transposase for random integration into the genome. Successful integrations were identified by fluorescence expression and selected for. Cells were subsequently differentiated for 10 days into myocytes to express the full-length DMD Dp427m muscle isoform. Then, RAB7A editing or DMD exon skipping was measured using droplet digital PCR. Untransfected RD cells after 10 days of myocyte differentiation were used as a negative control.

Existing antisense oligonucleotides operably linked to SmOPT and U7 hairpin sequences are currently being used for exon skipping therapies, and function by physically masking intronic and exonic splice enhancer sequences. To demonstrate that the novel promoters of the present disclosure can also improve activity in this capacity, antisense oligonucleotide sequences targeting clinically relevant Duchenne muscular dystrophy (DMD) exons were tested. For DMD exon 2 skipping, antisense sequences of GTTTTCTTTTGAACATCTTCTCTTTCATCTA (SEQ ID NO: 62) and ATTCTTACCTTAGAAAATTGTGC (SEQ ID NO: 63) were tested. Longer antisense sequences were also tested, which encompasses both SEQ ID NO: 62 and SEQ ID NO: 63 (CCATTCTTACCTTAGAAAATTGTGCATTTACCCATTTTGTGAATGTTTTCTTTTGAAC ATCTTCTCTTTCATCTA; SEQ ID NO: 64) and covers the entirety of DMD exon 2. For DMD exon 51 skipping, antisense sequences “long1” (GCAGGTACCTCCAACATCAAGGAAGATGGCATTTCTAGTTTGGAG; SEQ ID NO: 65) and “dt” (CCTCTGTGATTTTATAACTTGATTCAAGGAAGATGGCATTTCT; SEQ ID NO: 66) were tested. The antisense oligonucleotide of SEQ ID NO: 66 is notable since it anneals to two non-contiguous sections of DMD exon 51. These antisense sequences were tested with the original hU1 promoter (SEQ ID NO: 13) or a modified U7 promoter (SEQ ID NO: 17) and a termination sequence of SEQ ID NO: 60. This demonstrates that the modified U7 promoter of the present disclosure is compatible with various other RNA elements and an antisense payload intended to physically mask the target RNA site, to provide exon skipping.

### Example 10

### Screening for Promoters and Termination Sequences that Enhance Payload Expression and Target Editing

This example describes a screen to identify promoters and termination sequences that enhance RNA payload expression and target editing. Promoter and termination sequence constructs are expressed at single copy levels in a HEK293 cell line expressing a non-fluorescent GFP-G67R reporter. The promoter and termination sequence constructs encode a guide RNA payload that facilitates ADAR mediated RNA editing of the GFP-G67R reporter via deamination. The promoter sequence is positioned upstream of the payload sequence, and the termination sequence is positioned downstream of the payload sequence. Deamination of the 67^{th} codon of the reporter facilitated by the guide RNA payload reverts “AGA” to “GGA”, corresponding to an Arg to Gly amino acid change, and recovers GFP fluorescence. Fluorescence is positively correlated with editing of the target adenosine. The promoters and termination sequences are screened with two different guide RNA payloads, including a guide RNA 100 bases in length with the target adenosine (A) positioned across the 75^{th} base from the 5′ end of the guide RNA and comprising a macro-footprint of a 6/6 symmetric internal loop at the −6 position (6 bases upstream of the target A to be edited) and a 6/6 symmetric internal loop at the +30 position (30 bases downstream of the target A to be edited). The RNA payload further had an SmOPT variant sequence and a U7 hairpin sequence downstream of the guide RNA.

In a first screen, proximal sequence elements (PSEs) and 3′ box sequence elements are screened within the context of a mouse U7 promoter with an OCT-1 transcription factor binding sequence of SEQ ID NO: 28. The PSE sequence (SEQ ID NO: 22) is replaced with a PSE from TABLE 2 (SEQ ID NO: 67-SEQ ID NO: 120). The 3′ box sequence element within the termination sequence is selected from a 3′ box sequence from TABLE 3 (SEQ ID NO: 121-SEQ ID NO: 166). The PSEs and 3′ box sequence elements are screened in combination to identify PSE and 3′ box sequence elements that enhance payload expression and target editing. Five additional random sequences are included in each of the PSE sequence pool and the 3′ box sequence pool as negative controls.

In a second screen, endogenous promoters containing a distal sequence element (DSE) and a PSE are screened in combination with endogenous termination sequences containing a 3′ box sequence element. The promoters from TABLE 5 (SEQ ID NO: 167-SEQ ID NO: 707) are screened in combination with the termination sequences from TABLE 7 (SEQ ID NO: 708-SEQ ID NO: 1240) to identify promoter and termination sequence pairs that enhance payload expression and target editing. Ten additional random sequences are included in each of the promoter sequence pool and the termination sequence pool as negative controls.

In a third screen, PSEs and 3′ box sequence elements identified in the first screen as enhancing payload expression and target editing are inserted into the promoters and termination sequences, respectively, identified in the second screen. The PSEs of promoters identified in the second screen are replaced with PSEs identified in the first screen. The 3′ box sequence elements in the termination sequences identified in the second screen are replaced with 3′ box sequence elements identified in the first screen. The resulting engineered promoters and termination sequences are screened in combination to identify sequences that enhance payload expression and target editing.

### Example 11

### In Vivo Targeting of the SNCA 3′UTR for RNA Editing

This example describes the use of a promoter of the present disclosure to target the 3′UTR of the SNCA gene with two guide RNA for ADAR-mediated RNA editing. An AAV was used to deliver the guide RNA payload. The AAV vector encoding the two guide RNA payloads included an upstream promoter sequence of SEQ ID NO: 1241 driving expression of a first guide RNA, a SmOPT sequence, a U7 hairpin, and a downstream sequence of SEQ ID NO: 60 and also included an upstream promoter sequence of SEQ ID NO: 17 driving expression of a second guide RNA, an SmOPT sequence, a U7 hairpin, and a downstream sequence of SEQ ID NO: 1242. The guide RNAs targeted the 3′UTR of SNCA was administered in mice via intracerebroventricular injection. Up to 75% in vivo RNA editing was observed in mouse brain 4 weeks post-administration, demonstrating that the modified promoters and modified 3′box sequences of the present disclosure are capable of driving expression of guide RNAs that facilitate in vivo RNA editing.

### Example 12

### Modified Promoters and Truncated 3′Box Termination Sequences

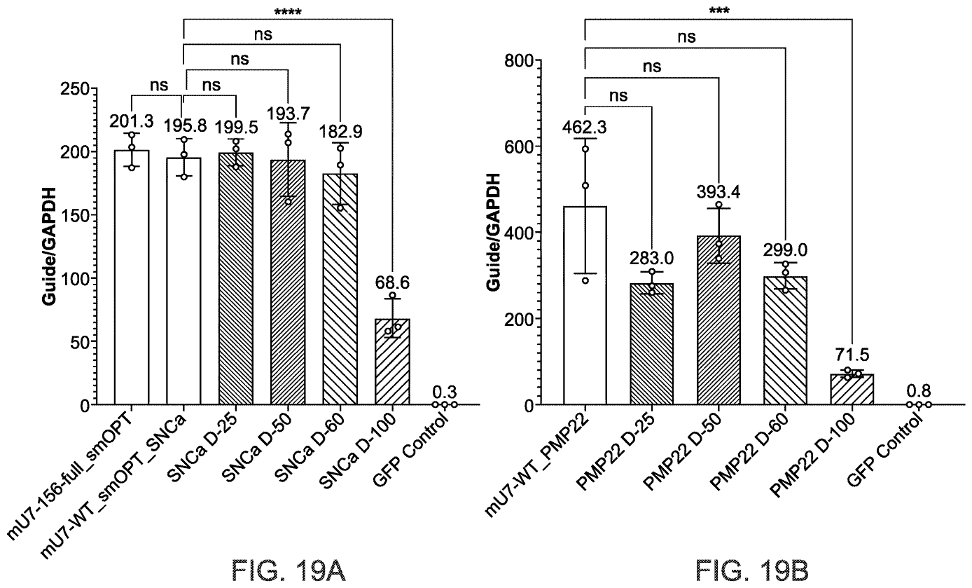

This example describes expression of gRNAs from AAV vector constructs in which a modified 3′box termination sequence was evaluated. Briefly vector plasmids comprising a wild type mU7 promoter or a variant of a wild type mU7 promoter driving expression of a guide RNA against a target RNA (SNCA or PMP22), an SmOPT sequence, a U7 hairpin sequence, and a downstream modified 3′box sequence were engineered and transiently transfected in cells. RNA was isolated 48 hours post transfection and treated with DNase. Guide RNA expression was quantified via ddPCR and normalized to a housekeeping gene (GAPDH). Results are shown in FIG. 19A and FIG. 19B. FIG. 19A shows data for constructs that have a wild type (WT) mU7 promoter variant of SEQ ID NO: 1248 (“mU7-156”) in which 100 bases between the DSE and PSE were deleted. All other constructs evaluated contained the wild type mU7 promoter of SEQ ID NO: 15.

The left two bars in FIG. 19A and the leftmost bar in of FIG. 19B assessed constructs containing the WT mU7 3′box termination sequence of SEQ ID NO: 1243. Constructs labeled D-25 had a downstream modified 3′box sequence comprising a sequence of SEQ ID NO: 1244, which was a 25-base truncation from the 3′end of the WT mU7 3′box termination sequence of SEQ ID NO: 1243. Constructs labeled D-50 had a downstream modified 3′box sequence comprising a sequence of SEQ ID NO: 1245, which was a 50-base truncation from the 3′end of the WT mU7 3′box termination sequence of SEQ ID NO: 1243. Constructs labeled D-60 had a downstream modified 3′box sequence comprising a sequence of SEQ ID NO: 1246, which was a 60-base truncation from the 3′end of the WT mU7 3′box termination sequence of SEQ ID NO: 1243. Constructs labeled D-100 had a downstream modified 3′box sequence comprising a sequence of SEQ ID NO: 1247, which was a 100-base truncation from the 3′end of the WT mU7 3′box termination sequence of SEQ ID NO: 1243.

As demonstrated in FIG. 19A and FIG. 19B, truncations of the WT mU7 3′box termination sequence resulted in guide RNA expression. Moreover, constructs comprising truncated WT mU7 3′box termination sequences of SEQ ID NO: 1244-SEQ ID NO: 1246 facilitated similar levels of guide RNA expression. Further, constructs comprising a variant of a wild type mU7 promoter of SEQ ID NO: 1248 in which 100 bases between the DSE and PSE were deleted also drove similar levels of guide RNA expression. Thus, these data demonstrate the modularity of the variant promoters and variant 3′box termination sequences disclosed herein in facilitating guide RNA expression.

### Example 13

### Additional Modified Promoter Sequences

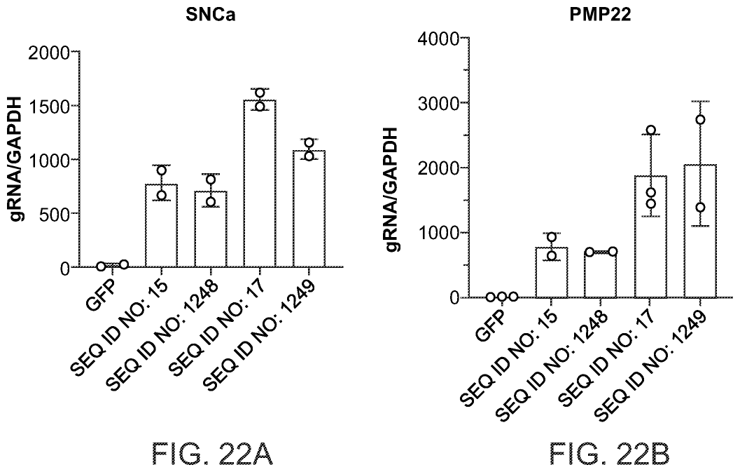

This example describes expression of gRNAs from AAV vector constructs in which promoters were modified by deletions of bases between the DSE and PSE. Briefly vector plasmids comprising full size and truncations of a wild type mU7 promoters or variants of a wild type mU7 promoter driving expression of a guide RNA against a target RNA (SNCA or PMP22), an SmOPT sequence, a U7 hairpin sequence, and a downstream modified 3′box sequence were engineered and transiently transfected in HEK293 cells. RNA was isolated 48 hours post transfection and treated with DNase. Guide RNA expression was quantified via ddPCR and normalized to a housekeeping gene (GAPDH). Results are shown in FIG. 22A and FIG. 22B. FIG. 22A and FIG. 22B show data for constructs that had a promoter sequence comprising a full-length WT mU7 promoter sequence (SEQ ID NO: 15), a variant of the WT mU7 promoter sequence with a 100 base deletion between the DSE and PSE promoter elements (SEQ ID NO: 1248), an engineered mU7 promoter sequence (SEQ ID NO: 17), or a variant of the engineered mU7 promoter sequence with a 100 base deletion between the DSE and PSE promoter elements (SEQ ID NO: 1249). As shown in FIG. 22A, the construct with the engineered mU7 promoter sequence (SEQ ID NO: 17) had the highest expression of the SNCA guide RNA. As shown in FIG. 22B, the constructs with the engineered mU7 promoter sequence (SEQ ID NO: 17) and the modified engineered mU7 promoter sequence with a 100 base deletion between the DSE and PSE promoter elements (SEQ ID NO: 1249) had the highest expression of the PMP22 guide RNA.

### Example 14

### RNA Editing of Modified Promoter Sequences

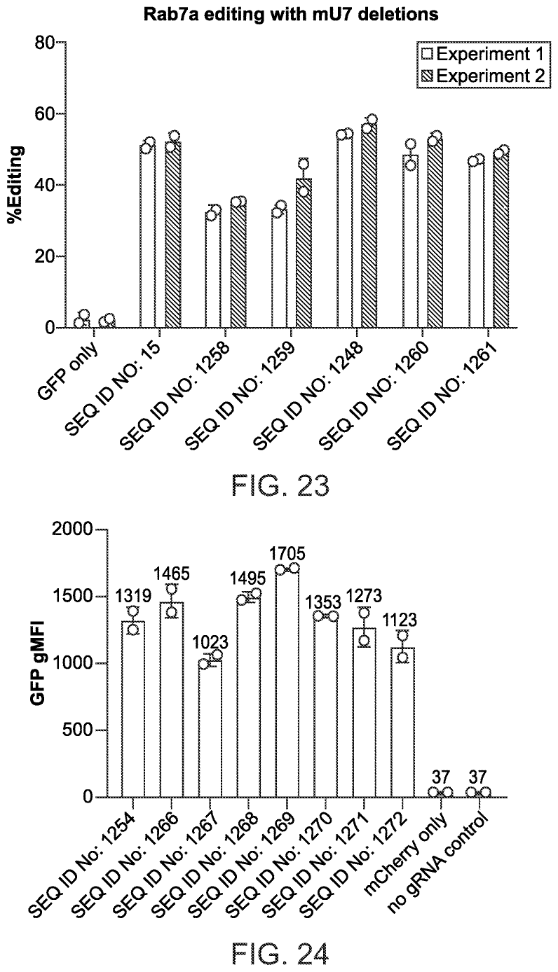

This example describes gRNA editing from AAV vector constructs in which promoters were modified by deletions of bases between the DSE and PSE. Briefly vector plasmids comprising full size and truncations of a wild type mU7 promoters driving expression of a Rab7a editing guide RNA against a target RNA (Rab7a), an SmOPT sequence, a U7 hairpin sequence, and a downstream modified 3′box sequence were engineered and transiently transfected in HEK293 cells. RNA was isolated 48 hours post transfection and treated with DNase. The isolated RNA was then converted into cDNA and the Rab7a editing was quantified (“% Editing” in FIG. 23). Results are shown in FIG. 23. FIG. 23 shows data for constructs that had a promoter sequence comprising a full-length WT mU7 promoter sequence (SEQ ID NO: 15), a variant of the WT mU7 promoter sequence with a 50 base deletion between the DSE and PSE promoter elements (SEQ ID NO: 1258), a variant of the WT mU7 promoter sequence with a 75 base deletion between the DSE and PSE promoter elements (SEQ ID NO: 1259), a variant of the WT mU7 promoter sequence with a 100 base deletion between the DSE and PSE promoter elements (SEQ ID NO: 1248), a variant of the WT mU7 promoter sequence with a 126 base deletion between the DSE and PSE promoter elements (SEQ ID NO: 1260), and a variant of the WT mU7 promoter sequence with a 135 base deletion between the DSE and PSE promoter elements (SEQ ID NO: 1261). As seen in FIG. 23, the percent editing of Rab7a was highest with a variant of the WT mU7 promoter sequence with a 100 base deletion between the DSE and PSE promoter elements (SEQ ID NO: 1248).

### Example 15

### Expression of Engineered Guide RNAs

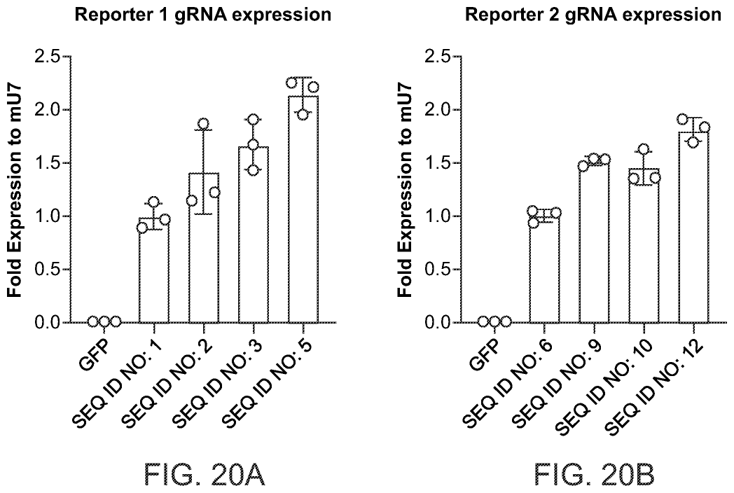

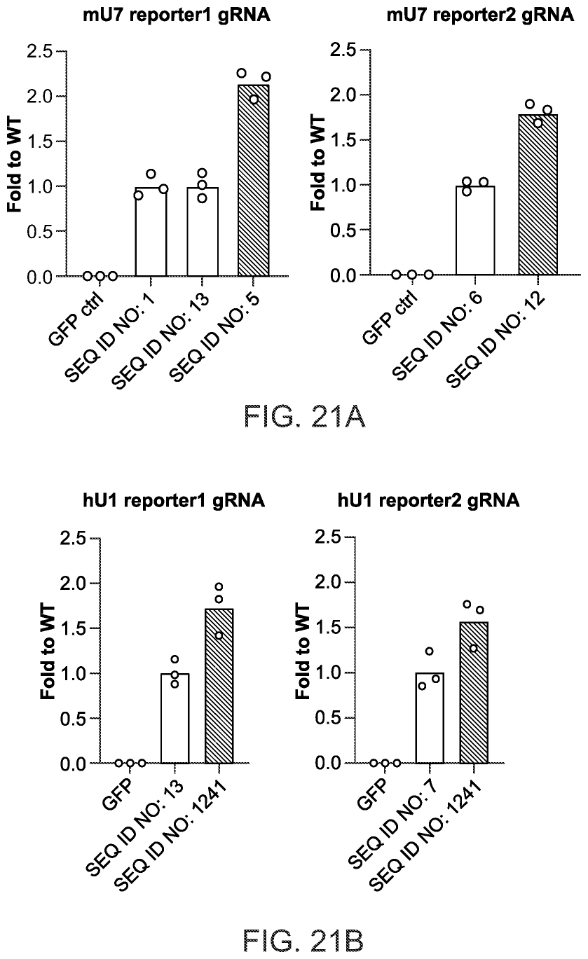

This example describes further evaluation of expression of engineered guide RNAs with the engineered promoter elements described herein (see EXAMPLE 6) and provided in TABLE 15. Constructs expressing either a PMP22-targeting engineered guide RNA (“Reporter 1” in FIG. 20A, FIG. 21A, FIG. 21B, and FIG. 22A) or an SNCA-targeting engineered guide RNA (“Reporter 2” in FIG. 20B, FIG. 21A, FIG. 21B, and FIG. 22A) were screened using the luciferase assay described in EXAMPLE 1. Expression of the SNCA-targeting guide RNA and the PMP22-targeting guide RNA were also tested under control of a wildtype human U1 promoter (SEQ ID NO: 13), a wildtype mouse U7 promoter (SEQ ID NO: 15), and an engineered human U1 promoter (SEQ ID NO: 1241). A construct encoding only a GFP cassette (“GFP ctrl”) was used as a negative control.

As shown in FIG. 20A, the PMP22-targeting engineered guide RNA constructs with the engineered promoter elements included in SEQ ID NO: 2, SEQ ID NO: 3, and SEQ ID NO: 5 had increased fold expression relative to the control mU7 wildtype guide RNA construct (SEQ ID NO: 1). As shown in FIG. 20B, the SNCA-targeting engineered guide RNA constructs with the engineered promoter elements included in SEQ ID NO: 9, SEQ ID NO: 10, and SEQ ID NO: 12 had increased fold expression relative to the control mU7 wildtype guide RNA construct (SEQ ID NO: 6).

The transcription site modifications were then overlaid to additional small nucleotide RNA (snRNA) promoters including the wildtype mU7 promoter (mU7, SEQ ID NO: 15) and the wildtype human U1 promoter (hU1, SEQ ID NO: 13) and tested in HEK293T cells. As shown on the left panel of FIG. 21A, in HEK293T cells, PMP22-targeting engineered guide RNA constructs with the engineered promoter elements included in SEQ ID NO: 5 had increased fold expression relative to the control mU7 wildtype guide RNA construct (SEQ ID NO: 1), as well as increased expression when compared to a control PMP22-targeting guide RNA under the control of a wildtype human U1 promoter (SEQ ID NO: 13). Similarly, as shown on the right panel of FIG. 21A, in HEK293T cells, the SNCA-targeting engineered guide RNA constructs with the engineered promoter elements included in SEQ ID NO: 12 had increased fold expression relative to the control mU7 wildtype guide RNA construct (SEQ ID NO: 6).

The wildtype human U1 promoter (SEQ ID NO: 13) was also used for transcription sites modifications to create an engineered human U1 promoter (SEQ ID NO: 1241). As shown in the left panel of FIG. 21B, in HEK293T cells, the engineered PMP22-targeting guide RNA under the control of the engineered hU1 promoter (SEQ ID NO: 1241) had greater fold expression relative to a control PMP22-targeting guide RNA under the control of the wildtype human U1 promoter (SEQ ID NO: 13). As shown on the right panel of FIG. 21B, in HEK293T cells, the engineered SNCA-targeting guide RNA under the control of the engineered hU1 promoter (SEQ ID NO: 1241) had greater fold expression relative to the control hU1 wildtype guide RNA construct (SEQ ID NO: 7). Therefore, as shown in FIG. 21A and FIG. 21B, the regulatory site changes of the present disclosure can be used to enhance the performance of standard mouse U7 and human U1 promoters, as measured by boosted gRNA expression in both cases. These results demonstrate that the regulatory site modifications disclosed herein have the ability to be used across type II snRNA promoters (e.g., U7 and U1).

Expanding from HEK293T cells, the transcription site modifications to the wildtype mU7 promoter (mU7, SEQ ID NO: 15) were also tested in ARPE-19 cells and HepG2 cells, as shown in FIG. 17A and FIG. 17B, respectively.

### Example 16

### Treatment of Parkinson's Disease using a LRRK2-Targeting Engineered Guide RNA Expression Construct

This example describes treatment of Parkinson's disease in a subject using a LRRK2-targeting engineered guide RNA expression construct. The subject has a mutation in LRRK2 associated with Parkinson's disease (e.g., a G to A mutation that results in a G2019S amino acid substitution). An engineered guide RNA expression construct encoding an engineered guide RNA that hybridizes to LRRK2, optionally operatively linked to smOPT, and under transcriptional control of an engineered promoter comprising one or more sequence variant of SEQ ID NO: 24-SEQ ID NO: 37 or SEQ ID NO: 67-SEQ ID NO: 120, a promoter of any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263, an engineered termination sequence comprising one or more sequence variant of SEQ ID NO: 38-SEQ ID NO: 42 or SEQ ID NO: 121-SEQ ID NO: 166, a termination sequence of any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289, or a combination thereof, is delivered to a cell of the subject. Optionally, the RNA expression cassette comprises an OCT-1 transcription factor binding sequence of SEQ ID NO: 28, a proximal sequence element of SEQ ID NO: 31, and a transcription termination sequence of SEQ ID NO: 41. The engineered guide RNA, upon hybridization to the target RNA and formation of the guide-target RNA scaffold, forms a micro-footprint, macro-footprint, or both. The LRRK2-targeting guide RNA, optionally operatively linked to smOPT, is expressed in a cell of the subject having a mutant LRRK2. The expressed engineered guide RNA hybridizes to the mutant LRRK2 RNA in the cell and recruits ADAR editing enzyme to the mutant LRRK2 RNA. The ADAR enzyme edits the mutant LRRK2 RNA and corrects the mutation in the LRRK2 RNA associated with Parkinson's disease, thereby treating the Parkinson's disease.

### Example 17

### Treatment of Facioscapulohumeral Muscular Dystrophy using a DUX4-Targeting Engineered Guide RNA Expression Construct

This example describes treatment of facioscapulohumeral muscular dystrophy (FSHD) in a subject using a DUX4-targeting engineered guide RNA expression construct. An engineered guide RNA is designed to target a region of DUX4 RNA (e.g., the polyA tail) that, when edited by ADAR, would result in RNA and protein knockdown. An engineered guide RNA expression construct encoding the engineered guide RNA that hybridizes to DUX4, optionally operatively linked to smOPT, and under transcriptional control of an engineered promoter comprising one or more sequence variant of SEQ ID NO: 24-SEQ ID NO: 37 or SEQ ID NO: 67-SEQ ID NO: 120, a promoter of any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263, a engineered termination sequence comprising one or more sequence variant of SEQ ID NO: 38-SEQ ID NO: 42 or SEQ ID NO: 121-SEQ ID NO: 166, a termination sequence of any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289, or a combination thereof, is delivered to a cell of the subject. Optionally, the RNA expression cassette comprises an OCT-1 transcription factor binding sequence of SEQ ID NO: 28, a proximal sequence element of SEQ ID NO: 31, and a transcription termination sequence of SEQ ID NO: 41. The engineered guide RNA, upon hybridization to the target RNA and formation of the guide-target RNA scaffold, forms a micro-footprint, macro-footprint, or both. The DUX4-targeting guide RNA, optionally operatively linked to smOPT, is expressed in a cell of the subject having a mutant DUX4. The expressed engineered guide RNA hybridizes to the DUX4 RNA in the cell and recruits ADAR editing enzyme to the DUX4 RNA. The ADAR enzyme edits the DUX4 RNA and knocks down DUX4 RNA and protein expression associated with FSHD, thereby treating the FSHD.

### Example 18

### Treatment of a Synucleinopathy using a SNCA-Targeting Engineered Guide RNA Expression Construct

This example describes treatment of a synucleinopathy, such as Parkinson's disease or Lewy body dementia, in a subject using a SNCA-targeting engineered guide RNA expression construct. An engineered guide RNA is designed to target a region of SNCA RNA (e.g., the TIS) that, when edited by ADAR, would result in RNA and protein knockdown. An engineered guide RNA expression construct encoding an engineered guide RNA that hybridizes to SNCA, optionally operatively linked to smOPT, and under transcriptional control of an engineered promoter comprising one or more sequence variant of SEQ ID NO: 24-SEQ ID NO: 37 or SEQ ID NO: 67-SEQ ID NO: 120, a promoter of any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263, a engineered termination sequence comprising one or more sequence variant of SEQ ID NO: 38-SEQ ID NO: 42 or SEQ ID NO: 121-SEQ ID NO: 166, a termination sequence of any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289, or a combination thereof, is delivered to a cell of the subject. Optionally, the RNA expression cassette comprises an OCT-1 transcription factor binding sequence of SEQ ID NO: 28, a proximal sequence element of SEQ ID NO: 31, and a transcription termination sequence of SEQ ID NO: 41. The engineered guide RNA, upon hybridization to the target RNA and formation of the guide-target RNA scaffold, forms a micro-footprint, macro-footprint, or both. The SNCA-targeting guide RNA, optionally operatively linked to smOPT, is expressed in a cell of the subject having a mutant SNCA. The expressed engineered guide RNA hybridizes to the SNCA RNA in the cell and recruits ADAR editing enzyme to the SNCA RNA. The ADAR enzyme edits the SNCA RNA and knocks down SNCA RNA and protein expression associated with the synucleinopathy, thereby treating the synucleinopathy.

### Example 19

### Treatment of Frontotemporal Dementia using a GRN-Targeting Engineered Guide RNA Expression Construct

This example describes treatment of frontotemporal dementia in a subject using a GRN-targeting engineered guide RNA expression construct. An engineered guide RNA expression construct encoding an engineered guide RNA that hybridizes to GRN, optionally operatively linked to smOPT, and under transcriptional control of an engineered promoter comprising one or more sequence variant of SEQ ID NO: 24-SEQ ID NO: 37 or SEQ ID NO: 67-SEQ ID NO: 120, a promoter of any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263, a engineered termination sequence comprising one or more sequence variant of SEQ ID NO: 38-SEQ ID NO: 42 or SEQ ID NO: 121-SEQ ID NO: 166, a termination sequence of any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289, or a combination thereof, is delivered to a cell of the subject. Optionally, the RNA expression cassette comprises an OCT-1 transcription factor binding sequence of SEQ ID NO: 28, a proximal sequence element of SEQ ID NO: 31, and a transcription termination sequence of SEQ ID NO: 41. The engineered guide RNA, upon hybridization to the target RNA and formation of the guide-target RNA scaffold, forms a micro-footprint, macro-footprint, or both. The GRN-targeting guide RNA, optionally operatively linked to smOPT, is expressed in a cell of the subject having a mutant GRN. The expressed engineered guide RNA hybridizes to target GRN RNA in the cell and recruits ADAR editing enzyme. The ADAR enzyme edits a target A of the target GRN RNA, increasing GRN protein expression, thereby treating the frontotemporal dementia.

### Example 20

### Treatment of a Tauopathy using a MAPT-Targeting Engineered Guide RNA Expression Construct

This example describes treatment of a tauopathy, such as Alzheimer's disease frontotemporal dementia, Parkinson's disease, progressive supranuclear palsy, corticobasal degeneration, or chronic traumatic encephalopathy, in a subject using a MAPT-targeting engineered guide RNA expression construct. An engineered guide RNA is designed to target a region of MAPT RNA (e.g., the TIS) that, when edited by ADAR, would result in RNA and protein knockdown. An engineered guide RNA expression construct encoding an engineered guide RNA that hybridizes to MAPT, optionally operatively linked to smOPT, and under transcriptional control of an engineered promoter comprising one or more sequence variant of SEQ ID NO: 24-SEQ ID NO: 37 or SEQ ID NO: 67-SEQ ID NO: 120, a promoter of any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263, a engineered termination sequence comprising one or more sequence variant of SEQ ID NO: 38-SEQ ID NO: 42 or SEQ ID NO: 121-SEQ ID NO: 166, a termination sequence of any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289, or a combination thereof, is delivered to a cell of the subject. Optionally, the RNA expression cassette comprises an OCT-1 transcription factor binding sequence of SEQ ID NO: 28, a proximal sequence element of SEQ ID NO: 31, and a transcription termination sequence of SEQ ID NO: 41. The engineered guide RNA, upon hybridization to the target RNA and formation of the guide-target RNA scaffold, forms a micro-footprint, macro-footprint, or both. The MAPT-targeting guide RNA, optionally operatively linked to smOPT, is expressed in a cell of the subject having a mutant MAPT. The expressed engineered guide RNA hybridizes to the target MAPT RNA in the cell and recruits ADAR editing enzyme. The ADAR enzyme edits a target A of the MAPT RNA, thereby treating the tauopathy.

### Example 21

### Treatment of Alpha-1 Antitrypsin Deficiency using a SERPINA1-Targeting Engineered Guide RNA Expression Construct

This example describes treatment of alpha-1 antitrypsin deficiency in a subject using a SERPINA1-targeting engineered guide RNA expression construct. The subject has a mutation in SERPINA1 associated with alpha-1 antitrypsin deficiency (e.g., a G to A mutation that results in an E342K amino acid substitution). An engineered guide RNA expression construct encoding an engineered guide RNA that hybridizes to SERPINA1, optionally operatively linked to smOPT, and under transcriptional control of an engineered promoter comprising one or more sequence variant of SEQ ID NO: 24-SEQ ID NO: 37 or SEQ ID NO: 67-SEQ ID NO: 120, a promoter of any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263, a engineered termination sequence comprising one or more sequence variant of SEQ ID NO: 38-SEQ ID NO: 42 or SEQ ID NO: 121-SEQ ID NO: 166, a termination sequence of any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289, or a combination thereof, is delivered to a cell of the subject. Optionally, the RNA expression cassette comprises an OCT-1 transcription factor binding sequence of SEQ ID NO: 28, a proximal sequence element of SEQ ID NO: 31, and a transcription termination sequence of SEQ ID NO: 41. The engineered guide RNA contains a base mismatch relative to the mutant SERPINA1 sequence such that a bulge or mismatch forms upon hybridization of the engineered guide RNA to a mutant SERPINA1 RNA. The SERPINA1-targeting guide RNA, optionally operatively linked to smOPT, is expressed in a cell of the subject having a mutant SERPINA1. The expressed engineered guide RNA hybridizes to the mutant SERPINA1 RNA in the cell and recruits ADAR editing enzyme to the mutant SERPINA1 RNA. The ADAR enzyme edits the mutant SERPINA1 RNA and corrects the mutation in the SERPINA1 RNA associated with alpha-1 antitrypsin deficiency, thereby treating the alpha-1 antitrypsin deficiency.

### Example 22

### Treatment of Alzheimer's Disease using an APP-Targeting Engineered Guide RNA Expression Construct

This example describes treatment of Alzheimer's disease in a subject using an APP-targeting engineered guide RNA expression construct. An engineered guide RNA is designed to target a secretase enzyme cleavage site in APP that, when edited by ADAR, would result in reduced levels of AB 40/AB 42 cleavage fragments associated with Alzheimer's disease. An engineered guide RNA expression construct encoding an engineered guide RNA that hybridizes to APP, optionally operatively linked to smOPT, and under transcriptional control of an engineered promoter comprising one or more sequence variant of SEQ ID NO: 24-SEQ ID NO: 37 or SEQ ID NO: 67-SEQ ID NO: 120, a promoter of any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263, a engineered termination sequence comprising one or more sequence variant of SEQ ID NO: 38-SEQ ID NO: 42 or SEQ ID NO: 121-SEQ ID NO: 166, a termination sequence of any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289, or a combination thereof, is delivered to a cell of the subject. Optionally, the RNA expression cassette comprises an OCT-1 transcription factor binding sequence of SEQ ID NO: 28, a proximal sequence element of SEQ ID NO: 31, and a transcription termination sequence of SEQ ID NO: 41. The engineered guide RNA contains a base mismatch relative to the mutant APP sequence such that a bulge or mismatch forms upon hybridization of the engineered guide RNA to a mutant APP RNA. The APP-targeting guide RNA, optionally operatively linked to smOPT, is expressed in a cell of the subject having a mutant APP. The expressed engineered guide RNA hybridizes to the target APP RNA in the cell and recruits ADAR editing enzyme. The ADAR enzyme edits a target A of the APP RNA, reducing formation of plaque-forming fragments (e.g., AB 40 or AB 42), thereby treating the Alzheimer's disease.

### Example 23

### Treatment of Stargardt Disease using an ABCA4-Targeting Engineered Guide RNA Expression Construct

This example describes treatment of Stargardt disease in a subject using an ABCA4-targeting engineered guide RNA expression construct. The subject has a mutation in ABCA4 associated with Stargardt disease (e.g., G1961E). An engineered guide RNA expression construct encoding an engineered guide RNA that hybridizes to ABCA4, optionally operatively linked to smOPT, and under transcriptional control of an engineered promoter comprising one or more sequence variant of SEQ ID NO: 24-SEQ ID NO: 37 or SEQ ID NO: 67-SEQ ID NO: 120, a promoter of any of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263, a engineered termination sequence comprising one or more sequence variant of SEQ ID NO: 38-SEQ ID NO: 42 or SEQ ID NO: 121-SEQ ID NO: 166, a termination sequence of any of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289, or a combination thereof, is delivered to a cell of the subject. Optionally, the RNA expression cassette comprises an OCT-1 transcription factor binding sequence of SEQ ID NO: 28, a proximal sequence element of SEQ ID NO: 31, and a transcription termination sequence of SEQ ID NO: 41. The engineered guide RNA contains a base mismatch relative to the mutant ABCA4 sequence such that a bulge or mismatch forms upon hybridization of the engineered guide RNA to a mutant ABCA4 RNA. The ABCA4-targeting guide RNA, optionally operatively linked to smOPT, is expressed in a cell of the subject having a mutant ABCA4. The expressed engineered guide RNA hybridizes to the mutant ABCA4 RNA in the cell and recruits ADAR editing enzyme to the mutant ABCA4 RNA. The ADAR enzyme edits the mutant ABCA4 RNA and corrects the mutation in the ABCA4 RNA associated with Stargardt disease, thereby treating the Stargardt disease.

### Example 24

### Screening of HSUR Termination Sequences

This example describes screening of Herpesvirus saimiri U-RNA elements (HSUR). The HSUR elements were extracted from NCBI NC_001350 and incorporated downstream of a gRNA cassette with a RNU5B1 promoter (SEQ ID NO: 1250) and a GFP gRNA which targets a GFP-G67R reporter wherein deamination of an AGA codon to GGA restores fluorescence in a correlative fashion. The HSUR elements are provided in TABLE 16.

TABLE 16
HSUR Elements

| Termi- | | |
|---|---|---|
| nator | SEQ ID | |
| Origin | NO: | Sequence |
| HSUR1 | SEQ ID | TCTAAGTAAGTTAAAAGTAGACTTTGGGTATTT |
| | NO: 1266 | ACCGAGATCTCTGCAAACACAGAACTTCTGTTC |
| | | TCAAGTGTATCATTTTATATCACTAGCTGTTAA |
| | | A |
| HSUR2 | SEQ ID | CCTTAAGTTTAAAAAAAGGTATCTGTGCTCTCA |
| | NO: 1267 | AGGCTTTAAACTTTGTGTTTAAAAGTTTTAGAG |
| | | CCTTGAGAGCACTTCTCTAAAACTAAAAATTGT |
| | | T |
| HSUR3 | SEQ ID | CCTTTAGAAGTTAAAAAACAGACGTTAAAACTT |
| | NO: 1268 | GTAAATTCTAGTATCAGTAGCTTTAAAACACAA |
| | | ACAAAAAATACACTAGAAAAATACAGCAAGATT |
| | | A |
| HSUR4 | SEQ ID | CTTAGTAAGTTTAAAAACAGAAAAAAAACCGTG |
| | NO: 1269 | TTGCTACAGCTATAAACTTCAAACATGCAGTTT |
| | | ATAGCAGTGGGCAACACGTCTCATCTCAAAAAT |
| | | T |
| HSUR5 | SEQ ID | CCTAAGTCAGTACAAAAACAGAAAGTCCGCGCT |
| | NO: 1270 | CTTACTGCTTGATACTTCAACAAGAAGTTACAG |
| | | CAGTGAGAGCGCTGCTACATTATTTAGAACTTC |
| | | C |
| HSUR6 | SEQ ID | CTGTTACTAGTTTAAAAACAGAAGTTGCTACTC |
| | NO: 1271 | GTTAAAAAGTACTAAACAAACAAGCTTTTTAAA |
| | | ACTTAGCTTTAAAAAATCAACAATAATTTTGAA |
| | | C |
| HSUR7 | SEQ ID | CTTCCGTAAGTAAAAAACAGAACTGTGCTTTAA |
| | NO: 1272 | ACTGTTTTTAACAGAAACGCCTTGCGTCAAAAT |
| | | GAAAGTTCTTAAGTAAAAGCGCTCGTATCAAAA |
| | | T |

The cassettes were introduced as single copy by BxbI integrase and enriched by puromycin for 14 days. The GFP expression was quantified by the geometric mean of fluorescence intensity (GFP gMFI) by flow cytometry and cells were gated for mCherry fluorescence upstream to enable graphing only of the cells which were positive for the cassette. As shown in FIG. 24, three termination sequences displayed a higher GFP gMFI compared to the RNU5B1 termination sequence (SEQ ID NO: 1254) with HSUR4 (SEQ ID NO: 1269) being the highest and a potential reference point for future studies.

### Example 24

### Screening of Select Promoter Sequence and Termination Sequence Combinations

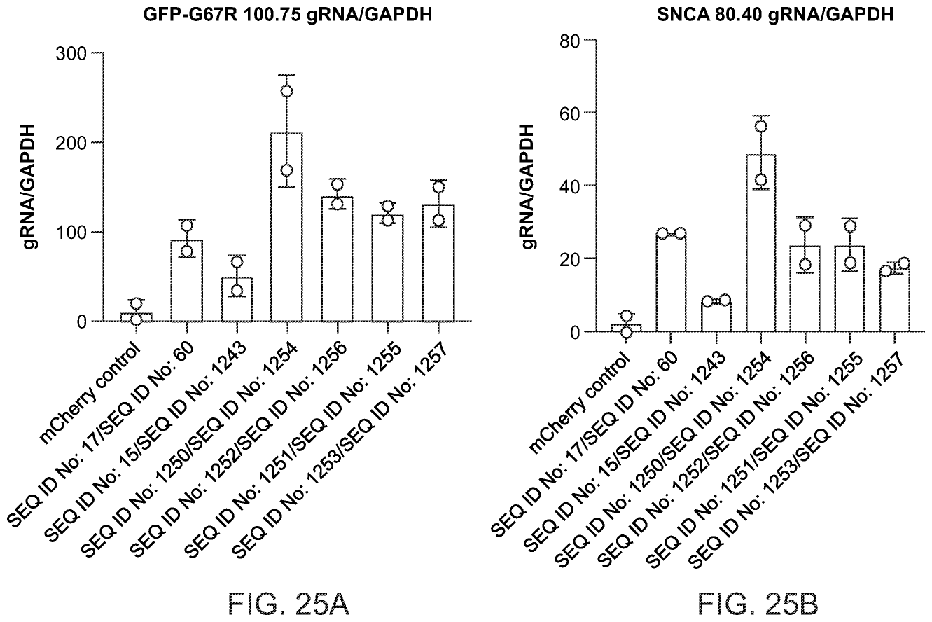

This example describes screening of select combinations of promoters and termination sequences. Briefly a subset of promoter sequences (TABLE 5) and termination sequences (TABLE 7) native to the human genome which followed canonical motif placement were experimentally tested in a single copy fashion against two target mRNAs. The promoter-termination sequence cassettes in question were paired with the endogenous counterparts. The promoter-termination sequence cassettes were compared against the promoter-termination sequence pair of a wildtype (WT) mU7 expression cassette with a promoter sequence of SEQ ID NO: 15 and a termination sequence of SEQ ID NO: 1243, and an engineered mU7 expression cassette with a promoter sequence of SEQ ID NO: 17 and a termination sequence of SEQ ID NO: 60. One of the targets was a GFP-G67R reporter wherein deamination of an AGA codon to GGA restores fluorescence in a correlative fashion. The other target was a model adenosine within the SNCA 3′ UTR. The gRNA expression was assessed by ddPCR. FIG. 25A shows the expression of the GFP-G67R gRNA expression cassette with a promoter sequence of SEQ ID NO: 17 and a termination sequence of SEQ ID NO: 60 (SEQ ID NO: 17/SEQ ID NO: 60), a promoter sequence of SEQ ID NO: 15 and a termination sequence of SEQ ID NO: 1243 (SEQ ID NO: 15/SEQ ID NO: 1243), a promoter sequence of SEQ ID NO: 1250 and a termination sequence of SEQ ID NO: 1254 (SEQ ID NO: 1250/SEQ ID NO: 1254), a promoter sequence of SEQ ID NO: 1252 and a termination sequence of SEQ ID NO: 1256 (SEQ ID NO: 1252/SEQ ID NO: 1256), a promoter sequence of SEQ ID NO: 1251 and a termination sequence of SEQ ID NO: 1255 (SEQ ID NO: 1251/SEQ ID NO: 1255), or a promoter sequence of SEQ ID NO: 1253 and a termination sequence of SEQ ID NO: 1257 (SEQ ID NO: 1253/SEQ ID NO: 1257). FIG. 25B shows the expression of the SNCA gRNA expression cassette with a promoter sequence of SEQ ID NO: 17 and a termination sequence of SEQ ID NO: 60 (SEQ ID NO: 17/SEQ ID NO: 60), a promoter sequence of SEQ ID NO: 15 and a termination sequence of SEQ ID NO: 1243 (SEQ ID NO: 15/SEQ ID NO: 1243), a promoter sequence of SEQ ID NO: 1250 and a termination sequence of SEQ ID NO: 1254 (SEQ ID NO: 1250/SEQ ID NO: 1254), a promoter sequence of SEQ ID NO: 1252 and a termination sequence of SEQ ID NO: 1256 (SEQ ID NO: 1252/SEQ ID NO: 1256), a promoter sequence of SEQ ID NO: 1251 and a termination sequence of SEQ ID NO: 1255 (SEQ ID NO: 1251/SEQ ID NO: 1255), or a promoter sequence of SEQ ID NO: 1253 and a termination sequence of SEQ ID NO: 1257 (SEQ ID NO: 1253/SEQ ID NO: 1257). As shown in FIG. 25A and FIG. 25B, the increased expression of both the GFP-G67R gRNA and the SNCA gRNA was seen in the expression cassettes with a promoter sequence of SEQ ID NO: 17 and a termination sequence of SEQ ID NO: 60 (SEQ ID NO: 17/SEQ ID NO: 60), a promoter sequence of SEQ ID NO: 1250 and a termination sequence of SEQ ID NO: 1254 (SEQ ID NO: 1250/SEQ ID NO: 1254), a promoter sequence of SEQ ID NO: 1252 and a termination sequence of SEQ ID NO: 1256 (SEQ ID NO: 1252/SEQ ID NO: 1256), a promoter sequence of SEQ ID NO: 1251 and a termination sequence of SEQ ID NO: 1255 (SEQ ID NO: 1251/SEQ ID NO: 1255), and a promoter sequence of SEQ ID NO: 1253 and a termination sequence of SEQ ID NO: 1257 (SEQ ID NO: 1253/SEQ ID NO: 1257) when compared to the WT mU7 expression cassette construct with a promoter sequence of SEQ ID NO: 15 and a termination sequence of SEQ ID NO: 1243 (SEQ ID NO: 15/SEQ ID NO: 1243). The top performing pairings were moved forward for individual sequence assessment as well as a reference point for future experiments.

### Example 25

### Pooled Screening of Termination Sequences by FlowSeq Screen

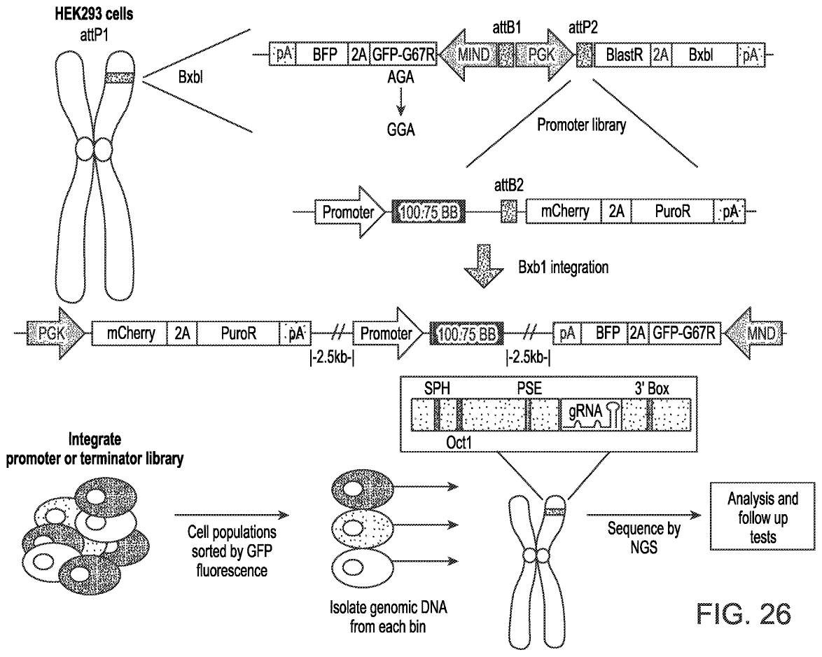

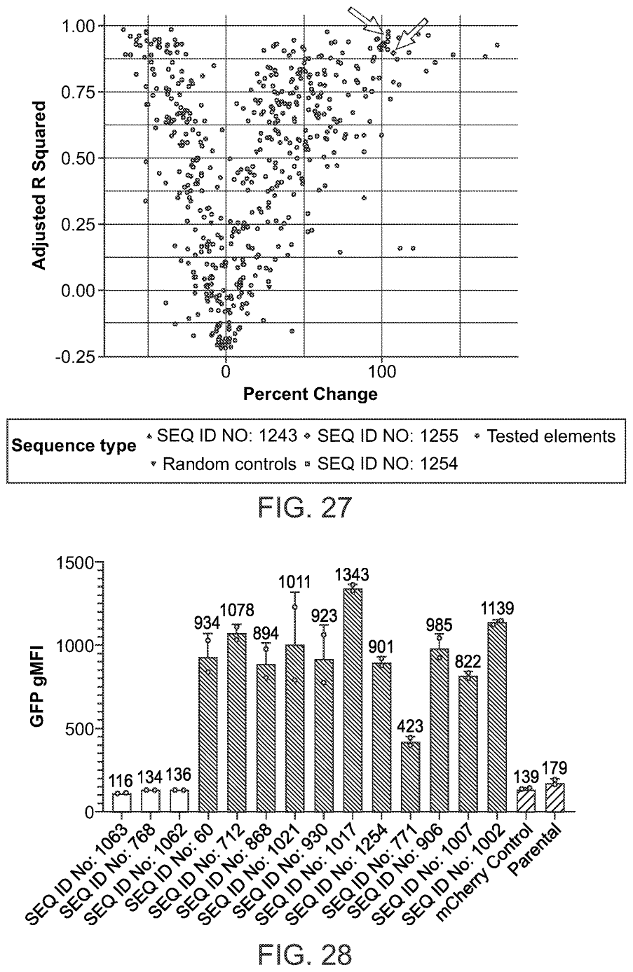

This example describes a flow-seq pipeline for screening of termination sequences. Briefly, as shown in FIG. 26, a library of 540 termination sequences were screened in a GFP-G67R Flowseq screen with a GFP gRNA. The library comprised putative termination sequences extracted from genomic sequences primarily downstream of human U1, U2, U4, U5, and U7 sequences which were cloned in triplicate downstream of three promoters with a GFP gRNA. The three promoters were SEQ ID NO:1250, SEQ ID NO: 17, and SEQ ID NO: 1251. The GFP gRNA targets a GFP-G67R reporter wherein deamination of an AGA codon to GGA restores fluorescence in a correlative fashion. The cassettes were introduced as single copy in a HEK293 reporter cell line by BxbI integrase and were enriched by puromycin until at least 90% of cells were positive as indicated by mCherry fluorescence. The GFP expression was quantified by the geometric mean of fluorescence intensity (GFP gMFI) by flow cytometry and cells were gated for mCherry fluorescence upstream to enable graphing only of the cells which were positive for the cassette. Once enriched, the cells were sorted into bins of fluorescence by a SONY SH800S cell sorter. The cells were sorted into two bins, the top 10% of cells and the bottom 10% of cells determined by GFP gMFI. Post sorting, the cells were confirmed to have a correspondingly increased or decreased gMFI signal. Genomic DNA from each bin, as well as the unsorted population was isolated and sequenced. A linear model was developed based the relative abundance for a given termination sequence between these bins. FIG. 27 shows results from the flowseq analysis, with the points representing the normalized performance of each termination sequence pooled from each of three promoter sequences. The circled data points indicate superior termination sequences that were advanced into a single copy assessment including SEQ ID NO: 1254 and SEQ ID NO: 1255 that showed similar expression compared to a WT mU7 termination sequence (SEQ ID NO: 1243). FIG. 28 shows the results of single copy assessment of each termination sequence. As seen in FIG. 28, expression cassettes with termination sequences of SEQ ID NO: 712, SEQ ID NO: 868, SEQ ID NO: 1021, SEQ ID NO: 930, SEQ ID NO: 1017, SEQ ID NO: 1254, SEQ ID NO: 906, SEQ ID NO: 1007, and SEQ ID NO: 1002 all had similar or greater expression as compared to the engineered mU7 termination sequence of SEQ ID NO: 60.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An expression cassette comprising:
a promoter sequence comprising a sequence having at least 80% sequence identity to any one of:
a) SEQ ID NO: 17, SEQ ID NO: 1250, or SEQ ID NO: 1262;
b) SEQ ID NO: 13 or SEQ ID NO: 15; or
c) SEQ ID NO: 1241, SEQ ID NO: 1251, SEQ ID NO: 1252, SEQ ID NO: 1253, or SEQ ID NO: 1263;
a payload sequence under transcriptional control of the promoter sequence, the payload sequence comprising a small RNA payload; and
a termination sequence comprising a sequence having at least 80% identity to any one of:
a) SEQ ID NO: 1002, SEQ ID NO: 1017, SEQ ID NO: 1264, or SEQ ID NO: 1265; or
b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269.

2. An expression cassette comprising:
a promoter sequence comprising a sequence having at least 80% sequence identity to any one of:
a) SEQ ID NO: 17, SEQ ID NO: 1250, or SEQ ID NO: 1262;
b) SEQ ID NO: 13 or SEQ ID NO: 15; or
c) SEQ ID NO: 1241, SEQ ID NO: 1251, SEQ ID NO: 1252, SEQ ID NO: 1253, or SEQ ID NO: 1263;
a payload sequence under transcriptional control of the promoter sequence, the payload sequence comprising a small RNA payload; and
a termination sequence.

3. An expression cassette comprising:
a promoter sequence;
a payload sequence under transcriptional control of the promoter sequence, the payload sequence comprising a small RNA payload; and
a termination sequence comprising a sequence having at least 80% identity to any one of:
a) SEQ ID NO: 1002, SEQ ID NO: 1017, SEQ ID NO: 1264, or SEQ ID NO: 1265; or
b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269.

4. An expression cassette comprising:
a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263;
a payload sequence under transcriptional control of the promoter sequence, the payload sequence comprising a small RNA payload; and
a termination sequence comprising a sequence having at least 80% identity to any one of SEQ ID NO: 60, SEQ ID NO: 708-SEQ ID NO: 1240, SEQ ID NO: 1242, SEQ ID NO:
1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289.

5. An expression cassette comprising:
a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263;
a payload sequence under transcriptional control of the promoter sequence, the payload sequence comprising a small RNA payload; and
a termination sequence.

6. An expression cassette comprising:
a promoter sequence;
a payload sequence under transcriptional control of the promoter sequence, the payload sequence comprising a small RNA payload; and
a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289.

7. The expression cassette of any one of claims 4-6, wherein the promoter sequence comprises a sequence having at least 90% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263.

8. The expression cassette of any one of claims 4-6, wherein the promoter sequence comprises a sequence having at least 95% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263.

9. The expression cassette of any one of claims 4-8, wherein the termination sequence comprises a sequence having at least 90% sequence identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289.

10. The expression cassette of any one of claims 4-8, wherein the termination sequence comprises a sequence having at least 95% sequence identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289.

11. The expression cassette of any one of claims 1-10, wherein the promoter sequence comprises SEQ ID NO: 17.

12. The expression cassette of any one of claims 1-10, wherein the promoter sequence comprises SEQ ID NO: 1262.

13. The expression cassette of any one of claims 1-10, wherein the promoter sequence comprises SEQ ID NO: 1250.

14. The expression cassette of any one of claims 1-10, wherein the promoter sequence comprises SEQ ID NO: 1251.

15. The expression cassette of any one of claims 1-10, wherein the promoter sequence comprises SEQ ID NO: 1252.

16. The expression cassette of any one of claims 1-10, wherein the promoter sequence comprises SEQ ID NO: 1253.

17. The expression cassette of any one of claims 1-16, wherein the termination sequence comprises SEQ ID NO: 1264.

18. The expression cassette of any one of claims 1-16, wherein the termination sequence comprises SEQ ID NO: 1265.

19. The expression cassette of any one of claims 1-16, wherein the termination sequence comprises SEQ ID NO: 1254.

20. The expression cassette of any one of claims 1-16, wherein the termination sequence comprises SEQ ID NO: 1255.

21. The expression cassette of any one of claims 1-16, wherein the termination sequence comprises SEQ ID NO: 1257.

22. The expression cassette of any one of claims 1-16, wherein the termination sequence comprises SEQ ID NO: 60.

23. The expression cassette of any one of claims 1-16, wherein the termination sequence comprises SEQ ID NO: 1242.

24. The expression cassette of any one of claims 1-16, wherein the termination sequence comprises SEQ ID NO: 1269.

25. The expression cassette of any one of claims 1-16, wherein the termination sequence comprises SEQ ID NO: 1017.

26. The expression cassette of any one of claims 1-25, wherein the small RNA payload comprises an engineered guide RNA capable of hybridizing to a target sequence.

27. The expression cassette of claim 26, wherein the engineered guide RNA is at least 70%, at least 75%, at least 80%, at least 85%, at least 90%, at least 95%, or 100% reverse complementary to the target sequence.

28. The expression cassette of claim 26 or claim 27, wherein the engineered guide RNA comprises at least one base pair mismatch relative to the target sequence.

29. The expression cassette of any one of claims 26-28, wherein the target sequence comprises an adenosine residue.

30. The expression cassette of any one of claims 26-29, wherein the target sequence is an RNA sequence.

31. The expression cassette of claim 30, wherein the RNA sequence is a mRNA or a pre-mRNA.

32. The expression cassette of any one of claims 26-31, wherein the target sequence comprises a G to A mutation relative to a wild type sequence.

33. The expression cassette of any one of claims 26-32, wherein the target sequence comprises a missense mutation or a nonsense mutation relative to a wild type sequence.

34. The expression cassette of any one of claims 26-33, wherein the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2).

35. The expression cassette of any one of claims 26-34, wherein the payload sequence comprises at least 80%, at least 85%, at least 90%, at least 95%, or 100% sequence identity to SEQ ID NO: 1273, SEQ ID NO: 1274, or SEQ ID NO: 61.

36. The expression cassette of any one of claims 1-35, wherein the small RNA payload comprises an antisense oligonucleotide, an siRNA, an shRNA, a miRNA, or a tracrRNA.

37. The expression cassette of any one of claims 1-36, wherein the small RNA payload is not less than 20 nucleotide residues and not more than 500 nucleotide residues long.

38. The expression cassette of any one of claims 1-37, wherein the small RNA payload is not less than 60 and not more than 100 residues long.

39. The expression cassette of any one of claims 1-37, wherein the small RNA payload is not less than 80 and not more than 120 residues long.

40. The expression cassette of any one of claims 1-37, wherein the small RNA payload is not less than 100 and not more than 140 residues long.

41. The expression cassette of any one of claims 1-37, wherein the small RNA payload is not less than 130 and not more than 170 residues long.

42. The expression cassette of any one of claims 1-41, wherein the payload sequence further comprises an Sm binding sequence or a hairpin sequence.

43. The expression cassette of claim 42, wherein the hairpin sequence comprises a U7 hairpin.

44. The expression cassette of claim 42 or claim 43, wherein the hairpin sequence comprises at least 80%, at least 85%, at least 90%, at least 95%, or 100% sequence identity to SEQ ID NO: 52 or SEQ ID NO: 54, or the Sm binding sequence comprises at least 80%, at least 85%, at least 90%, at least 95%, or 100% sequence identity to SEQ ID NO: 56 or SEQ ID NO: 58.

45. The expression cassette of any one of claims 1-44, wherein the expression cassette has a length of not less than 1300 nucleotide residues and not more than 2160 nucleotide residues.

46. The expression cassette of any one of claims 1-45, wherein the expression cassette comprises at least 80% sequence identity to a U1 sequence or a U7 sequence.

47. The expression cassette of claim 46, wherein the U1 sequence is a mouse U1 sequence or a human U1 sequence.

48. The expression cassette of claim 46, wherein the U7 sequence is a mouse U7 sequence or a human U7 sequence.

49. The expression cassette of any one of claims 1-48, wherein the promoter sequence comprises a zinc finger 143 motif capable of recruiting a ZNF143 transcription factor.

50. The expression cassette of any one of claims 1-49, wherein the promoter sequence comprises an OCT-1 transcription factor binding sequence capable of recruiting an OCT-1 transcription factor.

51. The expression cassette of any one of claims 1-50, wherein the promoter sequence comprises a proximal sequence element capable of recruiting a SNAPc.

52. The expression cassette of claim 51, wherein the proximal sequence element is capable of integrator dependent recruitment of RNA polymerase II.

53. The expression cassette of any one of claims 1-52, wherein the small RNA payload is capable of forming a guide-target RNA scaffold comprising a structural feature upon hybridization of the small RNA payload to a target sequence.

54. The expression cassette of claim 53, wherein the structural feature is a bulge, a mismatch, an internal loop, a hairpin, or combinations thereof.

55. The expression cassette of claim 54, wherein the structural feature comprises the bulge, and wherein the bulge is a symmetric bulge.

56. The expression cassette of claim 54, wherein the structural feature comprises the bulge, and wherein the bulge is an asymmetric bulge.

57. The expression cassette of claim 54, wherein the structural feature comprises the internal loop, and wherein the internal loop is a symmetric internal loop.

58. The expression cassette of claim 54, wherein the structural feature comprises the internal loop, and wherein the internal loop is an asymmetric internal loop.

59. The expression cassette of claim 54, wherein the structural feature comprises the hairpin, and wherein the hairpin is a recruitment hairpin or a non-recruitment hairpin.

60. The expression cassette of any one of claims 43-59, wherein the guide-target RNA scaffold comprises a Wobble base pair.

61. A recombinant polynucleotide encoding one or more of the expression cassettes of any one of claims 1-60.

62. The recombinant polynucleotide of claim 61, encoding two of the expression cassettes of any one of claims 1-60 comprising a first promoter, a second promoter, a first termination sequence, and a second termination sequence.

63. The recombinant polynucleotide of claim 62, wherein the first promoter and the second promoter are the same.

64. The recombinant polynucleotide of claim 62, wherein the first promoter and the second promoter are different.

65. The recombinant polynucleotide of any one of claims 62-64, wherein the first termination sequence and the second termination sequence are the same.

66. The recombinant polynucleotide of any one of claims 62-64, wherein the first termination sequence and the second termination sequence are different.

67. The recombinant polynucleotide of any one of claims 62-66 wherein the first promoter comprises SEQ ID NO: 17.

68. The recombinant polynucleotide of any one of claim 62 or claims 64-67, wherein the second promoter comprises SEQ ID NO: 1262.

69. The recombinant polynucleotide of any one of claims 62-68 wherein the first termination sequence comprises SEQ ID NO: 1264.

70. The recombinant polynucleotide of any one of claims 62-64 or claims 66-69, wherein the second termination sequence comprises SEQ ID NO: 1265.

71. The recombinant polynucleotide of claim 62 wherein (a) the first promotor sequence comprises SEQ ID NO: 17, the first termination sequence comprises SEQ ID NO: 1264, the second promotor sequence comprises SEQ ID NO: 1262 and the second termination sequence comprises SEQ ID NO: 1265; or (b) the first promotor sequence comprises SEQ ID NO: 17, the first termination sequence comprises SEQ ID NO: 1265, the second promotor sequence comprises SEQ ID NO: 1262 and the second termination sequence comprises SEQ ID NO: 1264.

72. A viral vector encapsidating the expression cassette of any one of claims 1-60 or the recombinant polynucleotide of any one of claims 61-71.

73. The viral vector of claim 72, wherein the viral vector comprises two or more, three or more, or four or more expression cassettes of any one of claims 1-60.

74. The viral vector of claim 72 or claim 73, wherein the viral vector is an adeno-associated viral vector.

75. The viral vector of claim 74, wherein the adeno-associated viral vector is selected from the group consisting of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, AAVhu68, chimeras thereof, and combinations thereof.

76. A pharmaceutical composition comprising the expression cassette of any one of claims 1-60, the recombinant polynucleotide of any one of claims 61-71, or the viral vector of any one of claims 72-75 and a pharmaceutically acceptable excipient, carrier, diluent, or combination thereof.

77. A method of expressing a small RNA payload in a cell, the method comprising delivering the expression cassette of any one of claims 1-60, the recombinant polynucleotide of any one of claims 61-71, the viral vector of any one of claims 72-75, or the pharmaceutical composition of claim 76 to a cell and expressing the small RNA payload encoded by the expression cassette in the cell.

78. A method of editing a target sequence, the method comprising:
delivering the expression cassette of any one of claims 1-60, the recombinant polynucleotide of any one of claims 61-71, the viral vector of any one of claims 72-75, or the pharmaceutical composition of claim 76 to a cell encoding the target sequence;
expressing the small RNA payload in the cell, wherein the small RNA payload comprises an engineered guide RNA capable of hybridizing to a target sequence;
forming a guide-target RNA scaffold upon hybridization of the small RNA payload to the target sequence;
recruiting an editing enzyme to the target sequence; and
editing the target sequence with the editing enzyme.

79. A method of editing a target sequence, the method comprising:
delivering an expression cassette to a cell encoding the target sequence, wherein the expression cassette comprises:
a promoter sequence comprising a sequence having at least 80% sequence identity to any one of:
a) SEQ ID NO: 17, SEQ ID NO: 1250, or SEQ ID NO: 1262;
b) SEQ ID NO: 13 or SEQ ID NO: 15; or
c) SEQ ID NO: 1241, SEQ ID NO: 1251, SEQ ID NO: 1252, SEQ ID NO: 1253, or SEQ ID NO: 1263;
a payload sequence under transcriptional control of the promoter sequence, the payload sequence comprising a small RNA payload; and
a termination sequence comprising a sequence having at least 80% identity to any one of:
a) SEQ ID NO: 1002, SEQ ID NO: 1017, SEQ ID NO: 1264, or SEQ ID NO: 1265; or
b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269;
expressing the small RNA payload in the cell;
forming a guide-target RNA scaffold upon hybridization of the small RNA payload to the target sequence;
recruiting an editing enzyme to the target sequence; and
editing the target sequence with the editing enzyme.

80. A method of editing a target sequence, the method comprising:
delivering an expression cassette to a cell encoding the target sequence, wherein the expression cassette comprises:
a promoter sequence comprising a sequence having at least 80% sequence identity to any one of:
a) SEQ ID NO: 17, SEQ ID NO: 1250, or SEQ ID NO: 1262;
b) SEQ ID NO: 13 or SEQ ID NO: 15; or
c) SEQ ID NO: 1241, SEQ ID NO: 1251, SEQ ID NO: 1252, SEQ ID NO: 1253, or SEQ ID NO: 1263;
a payload sequence under transcriptional control of the promoter sequence, the payload sequence comprising a small RNA payload; and
a termination sequence;
expressing the small RNA payload in the cell;
forming a guide-target RNA scaffold upon hybridization of the small RNA payload to the target sequence;
recruiting an editing enzyme to the target sequence; and
editing the target sequence with the editing enzyme.

81. A method of editing a target sequence, the method comprising:
delivering an expression cassette to a cell encoding the target sequence, wherein the expression cassette comprises:
a promoter sequence;
a payload sequence under transcriptional control of the promoter sequence, the payload sequence comprising a small RNA payload; and
a termination sequence comprising a sequence having at least 80% identity to any one of:
a) SEQ ID NO: 1002, SEQ ID NO: 1017, SEQ ID NO: 1264, or SEQ ID NO: 1265; or
b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269;
expressing the small RNA payload in the cell;
forming a guide-target RNA scaffold upon hybridization of the small RNA payload to the target sequence;
recruiting an editing enzyme to the target sequence; and
editing the target sequence with the editing enzyme.

82. A method of editing a target sequence, the method comprising:
delivering an expression cassette to a cell encoding the target sequence, wherein the expression cassette comprises:
a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263;
a payload sequence under transcriptional control of the promoter sequence, the payload sequence comprising a small RNA payload; and
a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289;
expressing the small RNA payload in the cell;
forming a guide-target RNA scaffold upon hybridization of the small RNA payload to the target sequence;
recruiting an editing enzyme to the target sequence; and
editing the target sequence with the editing enzyme.

83. A method of editing a target sequence, the method comprising:
delivering an expression cassette to a cell encoding the target sequence, wherein the expression cassette comprises:
a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263;
a payload sequence under transcriptional control of the promoter sequence, the payload sequence comprising a small RNA payload; and
a termination sequence;
expressing the small RNA payload in the cell;
forming a guide-target RNA scaffold upon hybridization of the small RNA payload to the target sequence;
recruiting an editing enzyme to the target sequence; and
editing the target sequence with the editing enzyme.

84. A method of editing a target sequence, the method comprising:
delivering an expression cassette to a cell encoding the target sequence, wherein the expression cassette comprises:
a promoter sequence;
a payload sequence under transcriptional control of the promoter sequence, the payload sequence comprising a small RNA payload; and
a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289;
expressing the small RNA payload in the cell;
forming a guide-target RNA scaffold upon hybridization of the small RNA payload to the target sequence;
recruiting an editing enzyme to the target sequence; and
editing the target sequence with the editing enzyme.

85. The method of any one of claims 77-84, wherein the promoter sequence comprises SEQ ID NO: 17.

86. The method of any one of claims 77-84, wherein the promoter sequence comprises SEQ ID NO: 1262.

87. The method of any one of claims 77-84, wherein the promoter sequence comprises SEQ ID NO: 1250.

88. The method of any one of claims 77-84, wherein the promoter sequence comprises SEQ ID NO: 1251.

89. The method of any one of claims 77-84, wherein the promoter sequence comprises SEQ ID NO: 1252.

90. The method of any one of claims 77-84, wherein the promoter sequence comprises SEQ ID NO: 1253.

91. The method of any one of claims 77-90, wherein the termination sequence comprises SEQ ID NO: 1264.

92. The method of any one of claims 77-90, wherein the termination sequence comprises SEQ ID NO: 1265.

93. The method of any one of claims 77-90, wherein the termination sequence comprises SEQ ID NO: 1254.

94. The method of any one of claims 77-90, wherein the termination sequence comprises SEQ ID NO: 1255.

95. The method of any one of claims 77-90, wherein the termination sequence comprises SEQ ID NO: 1257.

96. The method of any one of claims 77-90, wherein the termination sequence comprises SEQ ID NO: 60.

97. The method of any one of claims 77-90, wherein the termination sequence comprises SEQ ID NO: 1242.

98. The method of any one of claims 77-90, wherein the termination sequence comprises SEQ ID NO: 1269.

99. The method of any one of claims 77-90, wherein the termination sequence comprises SEQ ID NO: 1017.

100. The method of any one of claims 78-99, wherein the target sequence comprises a mutation relative to a wild type sequence.

101. The method of claim 100, wherein editing the target sequence corrects the mutation in the target sequence.

102. The method of claim 100 or claim 101, wherein the mutation is a missense mutation.

103. The method of claim 100 or claim 101, wherein the mutation is a nonsense mutation.

104. The method of any one of claims 100-103, wherein the mutation is a G to A mutation.

105. The method of any one of claims 100-104, wherein the mutation is associated with a disease.

106. The method of claim 105, wherein the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease.

107. The method of any one of claims 78-106, wherein the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2).

108. The method of claim 78-107, wherein editing the target sequence comprises editing an untranslated region of the target sequence.

109. The method of claim 108, wherein the untranslated region is a 5′ untranslated region or a 3′ untranslated region.

110. The method of claim 109, wherein the 3′ untranslated region is a polyadenylation sequence.

111. The method of any one of claims 78-110, wherein editing the target sequence comprises editing a translation initiation site.

112. The method of any one of claims 78-111, wherein editing the target sequence alters expression of the target sequence.

113. The method of claim 112, wherein editing the target sequence increases expression of the target sequence.

114. The method of claim 112, wherein editing the target sequence decreases expression of the target sequence.

115. A method of treating a disease in a subject, the method comprising:
administering to the subject a composition comprising the expression cassette of any one of claims 1-60, the recombinant polynucleotide of any one of claims 61-71, the viral vector of any one of claims 72-75, or the pharmaceutical composition of claim 76;
delivering the expression cassette to a cell of the subject; and
expressing the small RNA payload in the cell, thereby treating the disease.

116. A method of treating a disease in a subject, the method comprising:
administering to the subject a composition comprising an expression cassette comprising:
a promoter sequence comprising a sequence having at least 80% sequence identity to any one of:
a) SEQ ID NO: 17, SEQ ID NO: 1250, or SEQ ID NO: 1262;
b) SEQ ID NO: 13 or SEQ ID NO: 15; or
c) SEQ ID NO: 1241, SEQ ID NO: 1251, SEQ ID NO: 1252, SEQ ID NO: 1253, or SEQ ID NO: 1263;
a payload sequence under transcriptional control of the promoter sequence, the payload sequence comprising a small RNA payload; and
a termination sequence comprising a sequence having at least 80% identity to any one of:
a) SEQ ID NO: 1002, SEQ ID NO: 1017, SEQ ID NO: 1264, or SEQ ID NO: 1265; or
b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269;
delivering the expression cassette to a cell of the subject; and
expressing the small RNA payload in the cell, thereby treating the disease.

117. A method of treating a disease in a subject, the method comprising:
administering to the subject a composition comprising an expression cassette comprising:
a promoter sequence comprising a sequence having at least 80% sequence identity to any one of:
a) SEQ ID NO: 17, SEQ ID NO: 1250, or SEQ ID NO: 1262;
b) SEQ ID NO: 13 or SEQ ID NO: 15; or
c) SEQ ID NO: 1241, SEQ ID NO: 1251, SEQ ID NO: 1252, SEQ ID NO: 1253 or SEQ ID NO: 1263;
a payload sequence under transcriptional control of the promoter sequence, the payload sequence comprising a small RNA payload; and
a termination sequence;
delivering the expression cassette to a cell of the subject; and
expressing the small RNA payload in the cell, thereby treating the disease.

118. A method of treating a disease in a subject, the method comprising:
administering to the subject a composition comprising an expression cassette comprising:
a promoter sequence;
a payload sequence under transcriptional control of the promoter sequence, the payload sequence comprising a small RNA payload; and
a termination sequence comprising a sequence having at least 80% identity to any one of:
a) SEQ ID NO: 1002, SEQ ID NO: 1017, SEQ ID NO: 1264, or SEQ ID NO: 1265; or
b) SEQ ID NO: 60, SEQ ID NO: 771, SEQ ID NO: 930, SEQ ID NO: 1007, SEQ ID NO: 1021, SEQ ID NO: 1242, SEQ ID NO: 1254, SEQ ID NO: 1255, SEQ ID NO: 1257, or SEQ ID NO: 1269;
delivering the expression cassette to a cell of the subject; and
expressing the small RNA payload in the cell, thereby treating the disease.

119. A method of treating a disease in a subject, the method comprising:
administering to the subject a composition comprising an expression cassette comprising:
a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263;
a payload sequence under transcriptional control of the promoter sequence, the payload sequence comprising a small RNA payload; and
a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289;
delivering the expression cassette to a cell of the subject; and
expressing the small RNA payload in the cell, thereby treating the disease.

120. A method of treating a disease in a subject, the method comprising:
administering to the subject a composition comprising an expression cassette comprising:
a promoter sequence comprising a sequence having at least 80% sequence identity to any one of SEQ ID NO: 13-SEQ ID NO: 17, SEQ ID NO: 167-SEQ ID NO: 707, SEQ ID NO: 1241, SEQ ID NO: 1248-SEQ ID NO: 1253, or SEQ ID NO: 1259-SEQ ID NO: 1263;
a payload sequence under transcriptional control of the promoter sequence, the payload sequence comprising a small RNA payload; and
a termination sequence;
delivering the expression cassette to a cell of the subject; and
expressing the small RNA payload in the cell, thereby treating the disease.

121. A method of treating a disease in a subject, the method comprising:
administering to the subject a composition comprising an expression cassette comprising:
a promoter sequence;
a payload sequence under transcriptional control of the promoter sequence, the payload sequence comprising a small RNA payload; and
a termination sequence comprising a sequence having at least 80% identity to any one of SEQ ID NO: 60, SEQ ID NO: 708-SEQ ID NO: 1240, SEQ ID NO: 1242, SEQ ID NO: 1243-SEQ ID NO: 1247, SEQ ID NO: 1254-SEQ ID NO: 1257, SEQ ID NO: 1264-SEQ ID NO: 1272, SEQ ID NO: 1275, or SEQ ID NO: 1287-SEQ ID NO: 1289;
delivering the expression cassette to a cell of the subject; and
expressing the small RNA payload in the cell, thereby treating the disease.

122. The method of any one of claims 115-121, wherein the promoter sequence comprises SEQ ID NO: 17.

123. The method of any one of claims 115-121, wherein the promoter sequence comprises SEQ ID NO: 1262.

124. The method of any one of claims 115-121, wherein the promoter sequence comprises SEQ ID NO: 1250.

125. The method of any one of claims 115-121, wherein the promoter sequence comprises SEQ ID NO: 1251.

126. The method of any one of claims 115-121, wherein the promoter sequence comprises SEQ ID NO: 1252.

127. The method of any one of claims 115-121, wherein the promoter sequence comprises SEQ ID NO: 1253.

128. The method of any one of claims 115-127, wherein the termination sequence comprises SEQ ID NO: 1264.

129. The method of any one of claims 115-127, wherein the termination sequence comprises SEQ ID NO: 1265.

130. The method of any one of claims 115-127, wherein the termination sequence comprises SEQ ID NO: 1254.

131. The method of any one of claims 115-127, wherein the termination sequence comprises SEQ ID NO: 1255.

132. The method of any one of claims 115-127, wherein the termination sequence comprises SEQ ID NO: 1257.

133. The method of any one of claims 115-127, wherein the termination sequence comprises SEQ ID NO: 60.

134. The method of any one of claims 115-127, wherein the termination sequence comprises SEQ ID NO: 1242.

135. The method of any one of claims 115-127, wherein the termination sequence comprises SEQ ID NO: 1269.

136. The method of any one of claims 115-127, wherein the termination sequence comprises SEQ ID NO: 1017.

137. The method of any one of claims 115-136, wherein the disease is a synucleinopathy, Parkinson's disease, Lewy body dementia, multiple system atrophy, Charcot-Marie-Tooth disease, hereditary neuropathy with liability to pressure palsies, Yuan-Harel-Lupski syndrome, a tauopathy, Alzheimer's disease, frontotemporal dementia, progressive supranuclear palsy, corticobasal degeneration, chronic traumatic encephalopathy, autism, traumatic brain injury, Dravet syndrome, Crohn's disease, muscular dystrophy, B-cell leukemia, Dejerine-Sottas disease, Stargardt disease, alpha-1 antitrypsin deficiency, Tay-Sachs disease, cystic fibrosis, liposomal acid lipase deficiency, or Gaucher disease.

138. The method of any one of claims 115-137, wherein the small RNA payload comprises an engineered guide RNA that hybridizes to a target sequence, and wherein the cell encodes the target sequence.

139. The method of claim 138, wherein the target sequence encodes α-synuclein (SNCA), peripheral myelin protein 22 (PMP22), double homeobox 4 (DUX4), leucine rich repeat kinase 2 (LRRK2), Tau (MAPT), progranulin (GRN), a duplication of the PMP22 associated with Charcot-Marie-Tooth disease type 1A (CMT1A), ATP-binding cassette sub-family A member 4 (ABCA4), amyloid precursor protein (APP), alpha-1 antitrypsin (SERPINA1), hexosaminidase A (HEXA), cystic fibrosis transmembrane conductance regulator (CFTR), lipase A (LIPA), glucosylceramidase beta (GBA), PTEN-induced kinase 1 (PINK1), or methyl CpG binding protein 2 (MECP2).

140. The method of claim 138 or claim 139, further comprising forming a guide-target RNA scaffold upon hybridization of the engineered guide RNA to the target sequence, recruiting an editing enzyme to the target sequence, and editing the target sequence with the editing enzyme.

141. The method of any one of claims 138-140, wherein the target sequence comprises a mutation relative to a wild type sequence.

142. The method of claim 141, wherein editing the target sequence corrects the mutation in the target sequence.

143. The method of claim 141 or claim 142, wherein the mutation is a missense mutation.

144. The method of claim 141 or claim 142, wherein the mutation is a nonsense mutation.

145. The method of any one of claims 141-144, wherein the mutation is a G to A mutation.

146. The method of any one of claims 141-145, wherein the mutation is associated with the disease.

147. The method of any one of claims 140-146, wherein editing the target sequence comprises editing an untranslated region of the target sequence.

148. The method of claim 147, wherein the untranslated region is a 5′ untranslated region or a 3′ untranslated region.

149. The method of claim 148, wherein the 3′ untranslated region is a polyadenylation sequence.

150. The method of any one of claims 140-149, wherein editing the target sequence comprises editing a translation initiation site.

151. The method of any one of claims 140-150, wherein editing the target sequence alters expression of the target sequence.

152. The method of claim 151, wherein editing the target sequence increases expression of the target sequence.

153. The method of claim 151, wherein editing the target sequence decreases expression of the target sequence.

154. The method of any one of claims 78-114 or 140-153, wherein the guide-target RNA scaffold comprises a structural feature.

155. The method of claim 154, wherein the structural feature is a bulge, a mismatch, an internal loop, a hairpin, or combinations thereof.

156. The method of claim 155, wherein the structural feature comprises the bulge, and wherein the bulge is a symmetric bulge.

157. The method of claim 155, wherein the structural feature comprises the bulge, and wherein the bulge is an asymmetric bulge.

158. The method of any one of claims 155-157, wherein the structural feature comprises the internal loop, and wherein the internal loop is a symmetric internal loop.

159. The method of any one of claims 155-157, wherein the structural feature comprises the internal loop, and wherein the internal loop is an asymmetric internal loop.

160. The method of any one of claims 155-159, wherein the structural feature comprises the hairpin, and wherein the hairpin is a recruitment hairpin or a non-recruitment hairpin.

161. The method of any one of claims 78-114 or 140-160, wherein the guide-target RNA scaffold comprises a Wobble base pair.

162. The method of any one of claims 78-114 or 140-161, wherein the editing enzyme comprises an ADAR, an APOBEC, or a Cas nuclease.

163. The method of claim 162, wherein the ADAR comprises ADAR1, ADAR2, ADAR3, or combinations thereof.

164. The method of any one of claims 78-114 or 140-163, wherein the target sequence comprises RNA or DNA.

165. The method of any one of claims 78-114 or 140-164, wherein the target sequence is a mRNA or a pre-mRNA.

166. The method of any one of claims 78-114 or 140-165, wherein editing the target sequence comprises deamidating a nucleotide of the target sequence.

167. The method of any one of claims 78-114 or 140-166, wherein the target sequence is edited with an efficiency of at least 10%, at least 20%, or at least 25%.
